(19) United States
(12) Patent Application Publication
(10) Pub. No.: US 2023/0257723 A1
(43) Pub. Date: Aug. 17, 2023

(54) CRISPR/CAS9 THERAPIES FOR CORRECTING DUCHENNE MUSCULAR DYSTROPHY BY TARGETED GENOMIC INTEGRATION

(71) Applicant: Duke University, Durham, NC (US)

(72) Inventors: Charles A. Gersbach, Chapel Hill, NC (US); Adrian Pickar Oliver, Rougemont, NC (US)

(21) Appl. No.: 17/921,316

(22) PCT Filed: Apr. 27, 2021
(86) PCT No.: PCT/US2021/029424

§ 371 Date: Oct. 25, 2022

Related U.S. Application Data
(60) Provisional application No. 63/016,282, filed on Apr. 27, 2020.
(60) Provisional application No. 63/160,551, filed on Mar. 12, 2021.

(51) Int. Cl. C12N 9/22; C12N 15/86; A61P 21/00

(52) U.S. Cl. CPC C12N 9/22; C12N 15/86; A61P 21/00; C12N 2310/20; C12N 2750/14143; C12N 2320/33

(57) ABSTRACT

Disclosed herein arm CRISPR/Cas-based genome editing compositions and methods for treating Duchenne Muscular Dystrophy by restoring dystrophin function. The CRISPR/Cas-based genome editing systems may include a guide RNA (gRNA) targeting a fragment of a mutant dystrophin gene, a Cas protein or a fusion protein comprising the Cas protein, and a donor sequence comprising a fragment of a wild-type dystrophin gene.

### CROSS-REFERENCE TO RELATED APPLICATIONS

This application claims priority to U.S. Provisional Patent Application No. 63/016,282, fled Apr. 27, 2020, and U.S. Provisional Patent Application No. 63/160,551, filed Mar. 12, 2021, each of which is incorporated herein by reference in its entirety.

### STATEMENT REGARDING FEDERALLY SPONSORED RESEARCH

This invention was made with government support under grant R01AR069085 awarded by the National Institutes of Health. The government has certain rights in the invention.

### FIELD

The present disclosure is directed to CRISPR/Cas-based genome editing compositions and methods for treating Duchenne Muscular Dystrophy by restoring dystrophin function.

### INTRODUCTION

Duchenne muscular dystrophy (DMD) is the most prevalent lethal heritable childhood disease occurring in ˜1:5000 newborn males. Progressive muscle weakness leading to mortality in patients' mid-20s is a result of mutations in the dystrophin gene. In most cases (˜60%), the mutations consist of deletions in one or more of the 79 exons from the dystrophin gene, leading to disruption of the reading frame. Previous therapeutic strategies typically aim to generate expression of a truncated but partially functional dystrophin protein that recapitulates a genotype corresponding to Becker muscular dystrophy, which is associated with milder symptoms relative to DMD. For example, several groups have adapted the CRISPR/Cas9 technology for gene editing in cultured human DMD cells and the mdx mouse model of DMD to restore the dystrophin reading frame by deleting specific exons. However, there remains a need to develop gene editing strategies to restore the complete, fully functional dystrophin protein.

### SUMMARY

In an aspect, the disclosure relates to a CRISPR/Cas-based genome editing system comprising one or more vectors encoding a composition, the composition comprising: (a) a guide RNA (gRNA) targeting a fragment of a mutant dystrophin gene, wherein the gRNA hybridizes to a target sequence within intron 51 or intron 44 of the mutant dystrophin gene; (b) a Cas protein or a fusion protein comprising the Cas protein; and (c) a donor sequence comprising a fragment of a wild-type dystrophin gene, wherein the donor sequence comprises exon 52 of the wild-type dystrophin gene.

In a further aspect, the disclosure relates to a CRISPR/Cas-based genome editing system comprising: (a) a guide RNA (gRNA) targeting a fragment of a mutant dystrophin gene, wherein the gRNA hybridizes to a target sequence within intron 51 or intron 44 of the mutant dystrophin gene; (b) a Cas protein or a fusion protein comprising the Cas protein; and (c) a donor sequence comprising a fragment of a wild-type dystrophin gene, wherein the donor sequence comprises exon 52 of the wild-type dystrophin gene.

Another aspect of the disclosure provides a CRISPR/Cas-based genome editing system comprising one or more vectors encoding a composition. The composition may include (a) a guide RNA (gRNA) targeting a fragment of a mutant dystrophin gene; (b) a Cas protein or a fusion protein comprising the Cas protein; and (c) a donor sequence comprising a fragment of a wild-type dystrophin gene.

Another aspect of the disclosure provides a CRISPR/Cas-based genome editing system. The system may include (a) a guide RNA (gRNA) targeting a fragment of a mutant dystrophin gene: (b) a Cas protein or a fusion protein comprising the Cas protein; and (c) a donor sequence comprising a fragment of a wild-type dystrophin gene.

In some embodiments, the gRNA hybridizes to a target sequence within intron 51 or intron 44 of the mutant dystrophin gene. In some embodiments, the gRNA hybridizes to a target sequence within the polynucleotide sequence of SEQ ID NO: 128 or SEQ ID NO: 156. In some embodiments, the donor sequence comprises exon 52 of the wild-type dystrophin gene. In some embodiments, donor sequence comprises the polynucleotide sequence of SEQ ID NO: 53. In some embodiments, the fragment of the wild-type dystrophin gene is flanked on both sides by a gRNA spacer and/or a PAM sequence. In some embodiments, the gRNA targets an intron that is between exon 51 and exon 52 of the mutant dystrophin gene. In some embodiments, the donor sequence comprises multiple exons of the wild-type dystrophin gene or a functional equivalent thereof.

In some embodiments, the donor sequence comprises one or more exons selected from exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, exon 72, exon 73, exon 74, exon 75, exon 76, exon 77, exon 78, and exon 79 of the wild-type dystrophin gene or a functional equivalent thereof. In some embodiments, the donor sequence comprises exons 52-79 of the wild-type dystrophin gene or a functional equivalent thereof. In some embodiments, the donor sequence comprises exons 45-79 of the wild-type dystrophin gene or a functional equivalent thereof. In some embodiments, exon 52 of the mutant dystrophin gene is mutated or at least partially deleted from the dystrophin gene, or wherein exon 52 of the mutant dystrophin gene is deleted and the intron is juxtaposed to where the deleted exon 52 would be in a corresponding wild-type dystrophin gene. In some embodiments, the gRNA binds and targets a polynucleotide sequence comprising: (a) a sequence selected from SEQ ID NOs: 29-51, 87, 157-170; (b) a fragment of a sequence selected from SEQ ID NOs: 29-51, 87, 157-170; (c) a complement of a sequence selected from SEQ ID NOs: 29-51, 87, 157-170, or fragment thereof; (d) a nucleic acid that is substantially identical to a sequence selected from SEQ ID NOs: 29-51, 87, 157-170, or complement thereof; or (e) a nucleic acid that hybridizes under stringent conditions to a sequence selected from SEQ ID NOs: 29-51, 87, 157-170, complement thereof, or a sequence substantially identical thereto. In some embodiments, the gRNA binds and targets or is encoded by a polynucleotide sequence selected from SEQ ID NOs: 29-51, 87, 157-170, or a variant thereof. In some embodiments, the gRNA spacer comprises a sequence selected from SEQ ID NOs: 29-51, 87, 157-170. In some embodiments, the gRNA comprises a polynucleotide sequence selected from SEQ ID NOs: 64-86, 88, 171-184. In some embodiments, the gRNA binds or is encoded by a polynucleotide sequence selected from SEQ ID NOs: 35, 40, and 44, or the gRNA comprises a polynucleotide sequence selected from SEQ ID NOs: 70, 75, and 79. In some embodiments, the donor sequence comprises a polynucleotide sequence selected from SEQ ID NOs: 53-56, 154, and 155. In some embodiments, the donor sequence comprises a polynucleotide of SEQ ID NO: 55. In some embodiments, the donor sequence comprises a polynucleotide of SEQ ID NO: 56. In some embodiments, the Cas protein is a Streptococcus pyogenes Cas9 protein or a Staphylococcus aureus Cas9 protein. In some embodiments, the Cas protein comprises an amino acid sequence of SEQ ID NO: 18 or SEQ ID NO: 19. In some embodiments, the vector is a viral vector. In some embodiments, the vector is an Adeno-associated virus (AAV) vector. In some embodiments, the AAV vector is AAV1, AAV2, AAV3, AAV4, AAV5, AAV6, AAV7, AAV8, AAV9, AAV-10, AAV-11, AAV-12, AAV-13, or AAVrh.74 vector. In some embodiments, one of the one or more vectors comprises a polynucleotide sequence selected from SEQ ID NOs: 57-60 and 129-130. In some embodiments, the molar ratio between gRNA and donor sequence is 1:1, or 1:5, or from 5:1 to 1:10, or from 1:1 to 1:5.

Another aspect of the disclosure provides a recombinant polynucleotide encoding a donor sequence, wherein the donor sequence is flanked on both sides by a gRNA spacer and/or a PAM sequence. In some embodiments, the donor sequence comprises one or more exons selected from exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, exon 72, exon 73, exon 74, exon 75, exon 76, exon 77, exon 78, and exon 79 of a dystrophin gene. In some embodiments, the dystrophin gene is a human dystrophin gene.

In some embodiments, the system results in a dystrophin gene that encodes an in-frame transcript comprising an exon 51 joined with an exon comprising a sequence of SEQ ID NO: 53 or SEQ ID NO: 55, and with an intron therebetween. In some embodiments, the donor sequence comprises a polynucleotide sequence comprising exons 52-79 of the human dystrophin gene. In some embodiments, the donor sequence comprises the polynucleotide sequence of SEQ ID NO: 55 or SEQ ID NO: 56. In some embodiments, the recombinant polynucleotide comprises a sequence selected from SEQ ID NOs: 57-60.

Another aspect of the disclosure provides a vector comprising a recombinant polynucleotide as detailed herein.

Another aspect of the disclosure provides a cell comprising a recombinant polynucleotide of as detailed herein or a vector as detailed herein.

Another aspect of the disclosure provides a composition for restoring dystrophin function in a cell having a mutant dystrophin gene, the composition comprising a system as detailed herein, a recombinant polynucleotide as detailed herein, or a vector as detailed herein.

Another aspect of the disclosure provides a kit comprising a system as detailed herein, a recombinant polynucleotide as detailed herein, or a vector as detailed herein, or a composition as detailed herein.

Another aspect of the disclosure provides a method for restoring dystrophin function in a cell or a subject having a mutant dystrophin gene. The method may include contacting the cell or the subject with a system as detailed herein, a recombinant polynucleotide as detailed herein, or a vector as detailed herein, or a composition as detailed herein. In some embodiments, the method results in a dystrophin gene that encodes an in-frame transcript comprising an exon 51 joined with an exon comprising a sequence of SEQ ID NO: 53 or SEQ ID NO: 55, and with an intron therebetween.

Another aspect of the disclosure provides a method for restoring dystrophin function in a cell or a subject having a disrupted dystrophin gene caused by one or more deleted or mutated exons. The method may include contacting the cell or the subject with a system as detailed herein, a recombinant polynucleotide as detailed herein, or a vector as detailed herein, or a composition as detailed herein. In some embodiments, the method results in a dystrophin gene that encodes an in-frame transcript comprising an exon 51 joined with an exon comprising a sequence of SEQ ID NO: 53 or SEQ ID NO: 55, and with an intron therebetween. In some embodiments, dystrophin function is restored by inserting one or more wild-type exons of dystrophin gene corresponding to the one or more deleted or mutated exons. In some embodiments, the subject is suffering from Duchenne Muscular Dystrophy.

Another aspect of the disclosure provides a genome editing system for correcting a dystrophin gene. The system may include a donor sequence comprising exons 52-79 or exons 45-79 of the wild-type dystrophin gene. In some embodiments, the genome editing system further includes a nuclease selected from homing endonuclease, zinc finger nuclease, TALEN, and Cas protein.

The disclosure provides for other aspects and embodiments that will be apparent in light of the following detailed description and accompanying figures.

### BRIEF DESCRIPTION OF THE DRAWINGS

FIG. 1 is a schematic diagram of the dystrophin protein.

FIG. 2 is a schematic diagram of the exons encoding the dystrophin protein and various interactions in the cell.

FIG. 3A, FIG. 3B, FIG. 3C, FIG. 3D, FIG. 3E show that HITI-mediated exon 52 insertion restores full-length dystrophin in humanized hDMDΔ52/mdx primary myofibers. (FIG. 3A) Schematic of dual AAV vector approach for HITI-based exon 52 integration and correction of hDMDΔ52 mutation. Orange pentagon, Cas9/gRNA target sequence. Orange triangle, Cas9 cleavage site with PAM. (FIG. 3B) Primary myoblasts were isolated from hDMDΔ52/mdx skeletal muscle, co-transduced with AAV2 vectors at 1:1 and 1:5 (Cas9:gRNA-donor) vector genome ratios, and differentiated into myofibers. (FIG. 3C) Validation of correct gene knock-in by genomic PCR. (FIG. 3D) Validation of correct donor mRNA splicing by cDNA PCR. (FIG. 3E) Western blot for dystrophin and Cas9 shows restoration of full-length dystrophin expression.

FIG. 4A, FIG. 4B, FIG. 4C, FIG. 4D, FIG. 4E, FIG. 4F, FIG. 4G, FIG. 4H, FIG. 4I, FIG. 4J show that AAV-CRISPR targeted exon 52 integration restores full-length dystrophin in hDMDΔ52/mdx mouse skeletal muscle. (FIG. 4A) Adult hDMDΔ52/mdx male mice were co-injected in TA muscles with AAV9 vectors at 1:1 and 1:5 (Cas9:gRNA-donor) vector genome ratios. (FIG. 4B) No significant differences in AAV viral genomes per diploid genomes (vg/dg) quantification in TA tissue between corresponding treatment groups. (FIG. 4C) Validation of correct gene knock-in in TA tissue by genomic PCR. Black triangle, detected intact AAV-donor integration. (FIG. 4D) Schematic of potential on-target genomic edits that resulted from targeted DNA cleavage. (FIG. 4E) Unbiased Tn5 tagmentation-based sequencing analysis of the various on-target genomic edits in TA tissues. (FIG. 4F) Unbiased Tn5 tagmentation-based sequencing quantification of total on-target genomic edits in TA tissues. (FIG. 4G) Validation of correct donor mRNA splicing in TA tissue by cDNA PCR. (FIG. 4H) Higher levels of corrected dystrophin transcripts in TA tissue for g7-Ex52 treated mice quantified by ddPCR. (FIG. 4I) Western blot for dystrophin and Cas9 expression shows restoration of dystrophin expression. (FIG. 4J) Dystrophin immunofluorescence staining shows a greater percentage of dystrophin positive fibers in g7-Ex52 treated mice (scale bar, 200 μm; each dot represents mean of 5 images per mouse). One-way ANOVA, followed by Tukey's post hoc multiple comparisons test (**P<0.01 and *P<0.05; mean±SEM; n=4 mice).

FIG. 5A, FIG. 5B, FIG. 5C, FIG. 5D, FIG. 5E, FIG. 5F show that HITI-mediated superexon insertion restores full-length dystrophin in humanized hDMDΔ52/mdx primary myofibers. (FIG. 5A) Schematic of dual AAV vector approach for HITI-based superexon integration and correction of hDMDΔ52 mutation. Pentagon (with black star), Cas9/gRNA target sequence. Triangle, Cas9 cleavage site with PAM. Black hexagon, stop codon. (FIG. 5B) Primary myoblasts were isolated from hDMDΔ52/mdx skeletal muscle, co-transduced with AAV2 vectors at 1:1 and 1:5 (Cas9:gRNA-donor) vector genome ratios, and differentiated into myofibers. (FIG. 5C) Validation of correct gene knock-in by genomic PCR. (FIG. 5D) Validation of correct donor mRNA splicing by cDNA PCR. (FIG. 5E) Characterization of Superexon-corrected polyA tail using 3′ RACE with genome-specific primer (GSP) for 3× stop. (FIG. 5F) Western blot for dystrophin and Cas9 shows restoration of dystrophin expression for Ex52 and superexon treated samples.

FIG. 6A, FIG. 6B, FIG. 6C, FIG. 6D, FIG. 6E, FIG. 6F show that AAV-CRISPR targeted superexon integration restores full-length dystrophin in hDMDΔ52/mdx mouse skeletal muscle. (FIG. 6A) Adult hDMDΔ52/mdx male mice were co-injected in TA muscles with AAV9 vectors at 1:1 and 1:5 (Cas9:gRNA-donor) vector genome ratios. (FIG. 6B) No significant differences in AAV vector genomes per diploid genomes (vg/dg) quantification in TA tissue between corresponding treatment groups. (FIG. 6C) Unbiased Tn5 tagmentation-based sequencing analysis of the various on-target genomic edits in TA tissues. (FIG. 6D) Quantification of corrected dystrophin transcripts in TA tissue by ddPCR. (FIG. 6E) Western blot for dystrophin and Cas9 shows restoration of dystrophin expression. (FIG. 6F) Dystrophin immunofluorescence staining shows a significant increase in the percentage of dystrophin positive fibers in g7-Ex52 treated mice compared to scrambled non-targeted donor control mice (scale bar, 200 μm; each dot represents mean of 5 images per mouse). One-way ANOVA, followed by Tukey's post hoc multiple comparisons test (**P<0.01 and *P<0.05; mean±SEM; n=6 mice).

FIG. 7A, FIG. 78, FIG. 7C, FIG. 7D, FIG. 7E, FIG. 7F, FIG. 7G, FIG. 7H show that systemic delivery of AAV-CRISPR targeted integration strategies restore full-length dystrophin in hDMDΔ52/mdx mouse cardiac muscle. (FIG. 7A) Systemic facial vein co-injection in P2 neonate hDMDΔ52/mdx male mice with AAV9 vectors at 1:1 and 1:5 (Cas9:gRNA-donor) vector genome ratios. (FIG. 7B) No significant differences in AAV vector genomes per diploid genomes (vg/dg) quantification in cardiac (heart) or skeletal (diaphragm and TA) tissue between corresponding treatment groups. (FIG. 7C) Unbiased Tn5 tagmentation-based sequencing analysis of the various on-target genomic edits shows corrected integration at levels above background in cardiac tissue. (FIG. 7D) Higher levels of corrected dystrophin transcripts in heart tissue for treated mice quantified by ddPCR. (FIG. 7E) Unbiased Tn5 tagmentation-based sequencing analysis of the various on-target heart cDNA shows diverse transcript outcomes including aberrant splicing. (FIG. 7F) Western blot for dystrophin and Cas9 shows restoration of dystrophin expression in heart tissue. (FIG. 7G) Dystrophin immunofluorescence staining in heart tissue shows detection of dystrophin positive fibers in all treated mice, with a significant increase in the percentage of dystrophin positive fibers in g7-superexon (1:1) treated mice compared to scrambled non-targeted donor control mice (scale bar, 200 μm; each dot represents mean of 5 images per mouse). (FIG. 7H) Serum creatine kinase levels show a decrease in hDMDΔ52/mdx treated mice compared to diseased hDMDΔ52/mdx scrambled non-targeted donor control mice. One-way ANOVA, followed by Tukey's post hoc multiple comparisons test (**P<0.01 and *P<0.05; mean t SEM; n=6 mice).

FIG. 8A, FIG. 8B, FIG. 8C, FIG. 8D show gRNA screening and validation of HITI-mediated integration. (FIG. 8A) Schematic of SaCas9 gRNAs targeting within intron 51 upstream of exon52 were designed with 21 nt spacers. (FIG. 8B) Indel formation by individual gRNAs co-transfected with SaCas9 plasmid in HEK293T cells was measured by Surveyor assay, which showed highest editing activity with g3, g6, and g7. (FIG. 8C) Indel formation by individual gRNAs cloned with 19-23nt spacers co-transfected with SaCas9 plasmid in DMD patient myoblasts was measured by Surveyor assay. (FIG. 8D) Electroporation of hDMDΔ52/mdx primary myoblasts with SaCas9 and gRNA AAV plasmids resulted in detection of gene knock-in by PCR and Sanger sequencing.

FIG. 9A, FIG. 9B show unbiased genomic DNA edit characterization of treated hDMDΔ52/mdx TA tissue. (FIG. 9A) Stacked total editing quantification of gDNA editing events in TA tissue from one PBS control and all treated hDMDΔ52/mdx mice using genome-specific primers (GSPs) that prime upstream of the respective gRNA target sites. (FIG. 9B) Editing quantification of corrected, indel, inverted integration, and AAV integration gDNA editing events in TA tissue from one PBS control and all treated hDMDΔ52/mdx mice.

FIG. 10 shows the genome-wide specificity analysis of the g7 gRNA. Identification of the top potential human off-target sites was measured by genome-wide in vitro genomic DNA digestion with g7 and CHANGE-seq analysis. Nucleotides that match the target site are indicated with a dots. Nucleotides that differ from the target are shown for each site. The read count, gRNA sequence (spacer and PAM), and the human genome (hg19) coordinates of the observed on-target and off-target sequences are provided.

FIG. 11A, FIG. 11B show unbiased genomic DNA edit characterization of treated hDMDΔ52/mdx TA tissue. (FIG. 11A) Stacked total editing quantification of gDNA editing events in TA tissue from treated hDMDΔ52/mdx mice. (FIG. 11B) Editing quantification of corrected, indel, inverted integration, and AAV integration gDNA editing events in TA tissue from all treated hDMDΔ52/mdx mice.

FIG. 12A, FIG. 12B show the unbiased genomic DNA edit characterization of treated hDMDΔ52/mdx mice following systemic injection. (FIG. 12A) Stacked total editing quantification of gDNA editing events in heart, diaphragm, and TA tissue from treated hDMDΔ52/mdx mice. (FIG. 12B) Combined editing quantification of corrected, indel, inverted integration, and AAV integration gDNA editing events in heart, diaphragm, and TA tissue from all treated hDMDΔ52/mdx mice.

FIG. 13A, FIG. 13B show the unbiased transcript edit characterization of treated hDMDΔ52/mdx cardiac tissue. (FIG. 13A) Quantification of transcript editing events in cardiac tissue from one non-targeted donor control and all treated hDMDΔ52/mdx mice. (FIG. 13B) Schematic of frequent SaCas9-containing transcript reads demonstrating on-target aberrant splicing with AAV-SaCas9 construct sequences and confirmed with corresponding cardiac genomic reads containing aligned Cas9-coding sequences.

FIG. 14 shows the nested quantification and representative immunofluorescence staining for full-length dystrophin restoration in cardiac tissue. For all dystrophin positive fiber quantification, 5 randomized images were taken for each mouse sample and human dystrophin-positive and total fibers (anti-laminin) were counted. A representative image of cardiac tissue is provided for each treated mouse. Nested quantification values were used for statistical analysis (scale bar, 200 μm; each dot represents mean a single quantification per mouse). One-way ANOVA, followed by Tukey's post hoc multiple comparisons test (*P<0.05; mean±SEM; n=6 mice).

### DETAILED DESCRIPTION

Described herein are CRISPR/Cas-based gene/genome editing compositions and methods for treating Duchenne Muscular Dystrophy (DMD) by restoring dystrophin function. DMD is typically caused by deletions in the dystrophin gene that disrupt the reading frame. Many strategies to treat DMD aim to restore the reading frame by removing or skipping over an additional exon, as it has been shown that an internally truncated dystrophin protein can still be partially functional. Detailed herein are AAV-based Homology-Independent Targeted Integration (HITI)-mediated gene editing therapies for correcting the dystrophin gene. Specifically, the CRISPR/Cas9 gene editing technology was adapted to direct the targeted insertion of missing exons into the dystrophin gene. As a therapeutically relevant target, HITI-mediated genome editing strategies were optimized in a humanized mouse model of DMD in which exon 52 has been removed in mice carrying the full-length human dystrophin gene (hDMDΔ52/mdx mice). To achieve targeted integration, an AAV vector containing the deleted genome sequence including exon 52, or in some cases exons 52-79, or in some cases exons 45-79, is co-delivered with an AAV vector encoding Cas9/gRNA expression cassettes to achieve full-length dystrophin restoration in skeletal and cardiac muscles. The AAV delivery system is used to express Cas9 and gRNAs to generate a targeted genomic DSB and to deliver donor templates for NHEJ-mediated integration at the cut site. Targeted integration of the exon(s) in cultured cells is confirmed. Combined with AAV delivery, HITI-mediated strategies for targeted insertion of missing exons provides a method to restore full-length dystrophin and improve functional outcomes.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Binding region” as used herein refers to the region within a target region that is recognized and bound by the CRISPR/Cas-based gene editing system.

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, Tex.; SAS Institute Inc., Cary, N.C.). The healthy or normal levels or ranges for a target or for a protein activity may be defined in accordance with standard practice. A control may be a subject or cell without a composition as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Correcting”, “gene editing,” and “restoring” as used herein refers to changing a mutant gene that encodes a dysfunctional protein or truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Donor DNA”, “donor template,” and “repair template” as used interchangeably herein refers to a double-stranded DNA fragment or molecule that includes at least a portion of the gene of interest. The donor DNA may encode a full-functional protein or a partially functional protein.

“Duchenne Muscular Dystrophy” or “DMD” as used interchangeably herein refers to a recessive, fatal, X-linked disorder that results in muscle degeneration and eventual death. DMD is a common hereditary monogenic disease and occurs in 1 in 3500 males. DMD is the result of inherited or spontaneous mutations that cause nonsense or frame shift mutations in the dystrophin gene. The majority of dystrophin mutations that cause DMD are deletions of exons that disrupt the reading frame and cause premature translation termination in the dystrophin gene. DMD patients typically lose the ability to physically support themselves during childhood, become progressively weaker during the teenage years, and die in their twenties.

“Dystrophin” as used herein refers to a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane that is responsible for regulating muscle cell integrity and function. The dystrophin gene or “DMD gene” as used interchangeably herein is 2.2 megabases at locus Xp21. The primary transcription measures about 2,400 kb with the mature mRNA being about 14 kb. 79 exons code for the protein which is over 3500 amino acids.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Genome editing” or “gene editing” as used herein refers to changing a gene. Genome editing may include correcting or restoring a mutant gene or adding additional mutations. Genome editing may include knocking out a gene, such as a mutant gene or a normal gene. Genome editing may be used to treat disease or, for example, enhance muscle repair, by changing the gene of interest. In some embodiments, the compositions and methods detailed herein are for use in somatic cells and not germ line cells.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter. Promoters that target muscle-specific stem cells may include, for example, the CK8 promoter, the Spc5-12 promoter, and the MHCK7 promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product. The target gene may be a mutated gene involved in a genetic disease. The target gene may encode a known or putative gene product that is intended to be corrected or for which its expression is intended to be modulated. In certain embodiments, the target gene is the dystrophin gene or a portion thereof.

“Target region” as used herein refers to the region of the target gene to which the CRISPR/Cas9-based gene editing or targeting system is designed to bind.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

Transcriptional regulatory elements” or “regulatory elements” refers to a genetic element which can control the expression of nucleic acid sequences, such as activate, enhancer, or decrease expression, or alter the spatial and/or temporal expression of a nucleic acid sequence. Examples of regulatory elements include, for example, promoters, enhancers, splicing signals, polyadenylation signals, and termination signals. A regulatory element can be “endogenous,” “exogenous,” or “heterologous” with respect to the gene to which it is operably linked. An “endogenous” regulatory element is one which is naturally linked with a given gene in the genome. An “exogenous” or “heterologous” regulatory element is one which is not normally linked with a given gene but is placed in operable linkage with a gene by genetic manipulation.

“Treatment” or “treating” or “treatment” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease. As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome, or yeast artificial chromosome. A vector may be a DNA or RNA vector. A vector may be a self-replicating extrachromosomal vector, and preferably, is a DNA plasmid. For example, the vector may encode a Cas9 protein and at least one gRNA molecule.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. DYSTROPHIN

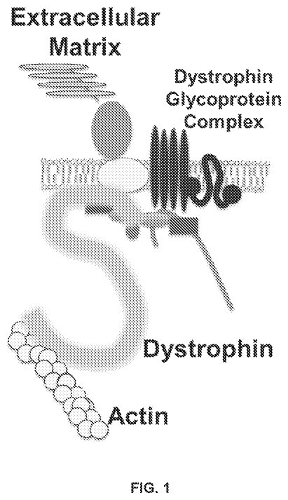

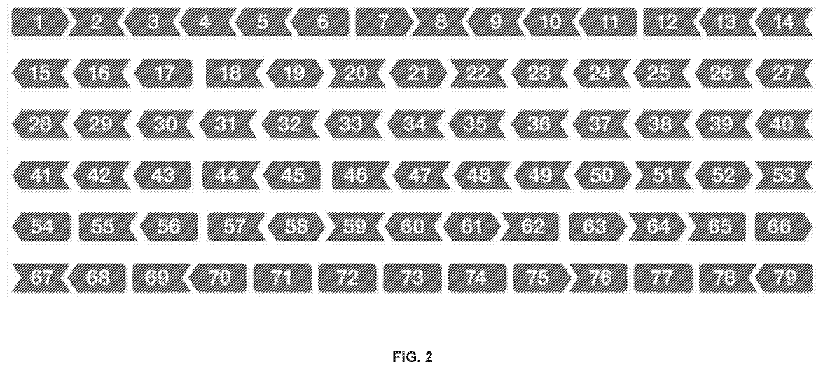

Dystrophin is a rod-shaped cytoplasmic protein and a part of a protein complex that connects the cytoskeleton of a muscle fiber to the surrounding extracellular matrix through the cell membrane (FIG. 1). Dystrophin provides structural stability to the dystroglycan complex of the cell membrane. The dystrophin gene is 2.2 megabases at locus Xp21. The primary transcription measures about 2,400 kb with the mature mRNA being about 14 kb. 79 exons include approximately 2.2 million nucleotides and code for the protein, which is over 3500 amino acids (FIG. 2). The large size of the dystrophin gene as well as its repetitive elements make the gene susceptible to recombination, leading to deletions of one or more exons. Normal skeleton muscle tissue contains only small amounts of dystrophin, but its absence of abnormal expression leads to the development of severe and incurable symptoms. Some mutations in the dystrophin gene lead to the production of defective dystrophin and severe dystrophic phenotype in affected patients. Some mutations in the dystrophin gene lead to partially-functional dystrophin protein and a much milder dystrophic phenotype in affected patients.

DMD is the result of inherited or spontaneous mutations that cause nonsense or frame shift mutations in the dystrophin gene. DMD is the most prevalent lethal heritable childhood disease and affects approximately one in 5,000 newborn males. DMD is characterized by progressive muscle weakness, often leading to mortality in subjects at age mid-twenties, due to the lack of a functional dystrophin gene. Most mutations are deletions in the dystrophin gene that disrupt the reading frame. Naturally occurring mutations and their consequences are relatively well understood for DMD. In-frame deletions that occur in the exon 45-55 regions contained within the rod domain can produce highly functional dystrophin proteins, and many carriers are asymptomatic or display mild symptoms. Exons 45-55 of dystrophin are a mutational hotspot. Furthermore, more than 60% of patients may be treated by targeting exons in this region of the dystrophin gene. Efforts have been made to restore the disrupted dystrophin reading frame in DMD patients by skipping non-essential exon(s) (for example, exon 45 skipping) during mRNA splicing to produce internally deleted but functional dystrophin proteins. One therapeutic aim may be to generate expression of a truncated, but partially functional, dystrophin protein that is similar to the product of the DMD gene in Becker muscular dystrophy (BMD) that is associated with milder symptoms relative to DMD. The deletion of internal dystrophin exon(s) (for example, deletion of exon 45) may retain the proper reading frame and can generate an internally truncated but partially functional dystrophin protein. Deletions between exons 45-55 of dystrophin can result in a phenotype that is much milder compared to DMD.

A dystrophin gene may be a mutant dystrophin gene. A dystrophin gene may be a wild-type dystrophin gene. A dystrophin gene may have a sequence that is functionally identical to a wild-type dystrophin gene, for example, the sequence may be codon-optimized but still encode for the same protein as the wild-type dystrophin. A mutant dystrophin gene may include one or more mutations relative to the wild-type dystrophin gene. Mutations may include, for example, nucleotide deletions, substitutions, additions, transversions, or combinations thereof. Mutations may be in one or more exons and/or introns. Mutations may include deletions of all or parts of at least one intron and/or exon. An exon of a mutant dystrophin gene may be mutated or at least partially deleted from the dystrophin gene. An exon of a mutant dystrophin gene may be fully deleted. A mutant dystrophin gene may have a portion or fragment thereof that corresponds to the corresponding sequence in the wild-type dystrophin gene. In some embodiments, a disrupted dystrophin gene caused by a deleted or mutated exon can be restored in DMD patients by adding back the corresponding wild-type exon. In some embodiments, disrupted dystrophin caused by, for example, a deleted or mutated exon 52, can be restored in DMD patients by adding back in wild-type exon 52. In certain embodiments, exon 52 of a dystrophin gene refers to the 52nd exon of the dystrophin gene. Exon 52 is frequently adjacent to frame-disrupting deletions in DMD patients. Addition of exon 52 to restore the reading frame may ameliorate the phenotype in DMD subjects, including DMD subjects with deletion mutations. In certain embodiments, one or more exons may be added and inserted into the disrupted dystrophin gene. The one or more exons may be added and inserted so as to restore the corresponding mutated or deleted exon(s) in dystrophin. The one or more exons may be added and inserted into the disrupted dystrophin gene in addition to adding back and inserting the exon 52. In some embodiments, the one or more exons added and inserted into the disrupted dystrophin gene include exons 52-79. In some embodiments, the one or more exons added and inserted into the disrupted dystrophin gene include exons 45-79.

### 3. CRISPR/CAS9-BASED GENE EDITING SYSTEM

The compositions and methods detailed herein may be suitable for any gene editing system or tool wherein two targeting nucleases are combined to create a deletion in a genome. Gene editing systems may include, for example, those comprising homing endonucleases, zinc finger nucleases (ZFNs), transcription activator-like effector (TALE) nucleases (TALENs), and clustered regularly interspaced short palindromic repeats (CRISPR)-CRISPR-associated protein (Cas protein) such as Cas9. Homing endonucleases generally cleave their DNA substrates as dimers and do not have distinct binding and cleavage domains. ZFNs recognize target sites that consist of two zinc-finger binding sites that flank a 5- to 7-base pair (bp) spacer sequence recognized by the FokI cleavage domain. TALENs recognize target sites that consist of two TALE DNA-binding sites that flank a 12- to 20-bp spacer sequence recognized by the FokI cleavage domain. In some embodiments, the compositions and methods detailed herein may be used with CRISPR/Cas9-based gene editing systems. Provided herein are CRISPR/Cas9-based gene editing systems. The CRISPR/Cas9-based gene editing system may be used to restore dystrophin gene function. The CRISPR/Cas9-based gene editing system may include a Cas9 protein or a fusion protein, and at least one gRNA.

“Clustered Regularly Interspaced Short Palindromic Repeats” and “CRISPRs,” as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures. Cas9 forms a complex with the 3′ end of a sgRNA (which may be referred interchangeably herein as “gRNA”), and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase Ill. This cleavage is followed by a second cleavage event within each spacer by Cas9, producing mature crRNAs that remain associated with the tracrRNA and Cas9, forming a Cas9:crRNA-tracrRNA complex.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Type II systems have differing PAM requirements.

An engineered form of the Type II effector system of S. pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in gene editing and treating genetic diseases. The CRISPR/Cas9-based engineered systems can be designed to target any gene, including genes involved in, for example, a genetic disease, aging, tissue regeneration, or wound healing. The CRISPR/Cas9-based gene editing system can include a Cas9 protein or a Cas9 fusion protein.

a. Cas9 Protein

Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula manina, Bradyrhizobium sp., Brevibacillus laterosporus, Campylobacter coli, Campylobacter jejuni, Campylobacter lari, candidatus punicei spirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In certain embodiments, the Cas9 molecule is a Streptococcus pyogenes Cas9 molecule (also referred herein as “SpCas9”). SpCas9 may comprise an amino acid sequence of SEQ ID NO: 18. In certain embodiments, the Cas9 molecule is a Staphylococcus aureus Cas9 molecule (also referred herein as “SaCas9”). SaCas9 may comprise an amino acid sequence of SEQ ID NO: 19.

A Cas9 molecule or a Cas9 fusion protein can interact with one or more gRNA molecule(s) and, in concert with the gRNA molecule(s), can localize to a site that comprises a target domain, and in certain embodiments, a PAM sequence. The Cas9 protein forms a complex with the 3′ end of a gRNA. The ability of a Cas9 molecule or a Cas9 fusion protein to recognize a PAM sequence can be determined, for example, by using a transformation assay as known in the art.

The specificity of the CRISPR-based system may depend on two factors: the target sequence and the protospacer-adjacent motif (PAM). The targeting sequence is located on the 5′ end of the gRNA and is designed to bond with base pairs on the host DNA at the correct DNA sequence known as the protospacer or target sequence. By simply exchanging the recognition sequence of the gRNA, the Cas9 protein can be directed to new genomic targets. The PAM sequence is located on the DNA to be altered and is recognized by a Cas9 protein. PAM recognition sequences of the Cas9 protein can be species specific.

In certain embodiments, the ability of a Cas9 molecule or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (for example, PAM sequences). A Cas9 molecule of S. pyogenes may recognize the PAM sequence of NRG (5′-NRG-3′, where R is any nucleotide residue, and in some embodiments, R is either A or G, SEQ ID NO: 1). In certain embodiments, a Cas9 molecule of S. pyogenes may naturally prefer and recognize the sequence motif NGG (SEQ ID NO: 2) and direct cleavage of a target nucleic acid sequence 1 to 10, for example, 3 to 5, bp upstream from that sequence. In some embodiments, a Cas9 molecule of S. pyogenes accepts other PAM sequences, such as NAG (SEQ ID NO: 3) in engineered systems (Hsu et al., Nature Biotechnology 2013 doi:10.1038/nbt.2647). In certain embodiments, a Cas9 molecule of S. thermophilus recognizes the sequence motif NGGNG (SEQ ID NO: 4) and/or NNAGAAW (W=A or T) (SEQ ID NO: 5) and directs cleavage of a target nucleic acid sequence 1 to 10, for example, 3 to 5, bp upstream from these sequences. In certain embodiments, a Cas9 molecule of S. mutans recognizes the sequence motif NGG (SEQ ID NO: 2) and/or NAAR (R=A or G) (SEQ ID NO: 6) and directs cleavage of a target nucleic acid sequence 1 to 10, for example, 3 to 5 bp, upstream from this sequence. In certain embodiments, a Cas9 molecule of S. aureus recognizes the sequence motif NNGRR (R=A or G) (SEQ ID NO: 7) and directs cleavage of a target nucleic acid sequence 1 to 10, for example, 3 to 5, bp upstream from that sequence. In certain embodiments, a Cas9 molecule of S. aureus recognizes the sequence motif NNGRRN (R=A or G) (SEQ ID NO: 8) and directs cleavage of a target nucleic acid sequence 1 to 10, for example, 3 to 5, bp upstream from that sequence. In certain embodiments, a Cas9 molecule of S. aureus recognizes the sequence motif NNGRRT (R=A or G) (SEQ ID NO: 9) and directs cleavage of a target nucleic acid sequence 1 to 10, for example, 3 to 5, bp upstream from that sequence. In certain embodiments, a Cas9 molecule of S. aureus recognizes the sequence motif NNGRRV (R=A or G; V=A or C or G) (SEQ ID NO: 10) and directs cleavage of a target nucleic acid sequence 1 to 10, for example, 3 to 5, bp upstream from that sequence. A Cas9 molecule derived from Neisseria meningitidis (NmCas9) normally has a native PAM of NNNNGATT (SEQ ID NO: 11), but may have activity across a variety of PAMs, including a highly degenerate NNNNGNNN PAM (SEQ ID NO: 12) (Esvelt et al. Nature Methods 2013 doi:10.1038/nmeth.2681). In the aforementioned embodiments, N can be any nucleotide residue, for example, any of A, G, C, or T. Cas9 molecules can be engineered to alter the PAM specificity of the Cas9 molecule.

In some embodiments, the Cas9 protein recognizes a PAM sequence of or comprising NGG (SEQ ID NO: 2) or NGA (SEQ ID NO: 13) or NNNRRT (R=A or G) (SEQ ID NO: 14) or ATTCCT (SEQ ID NO: 15) or NGAN (SEQ ID NO: 16) or NGNG (SEQ ID NO: 17). In some embodiments, the Cas9 protein is a Cas9 protein of S. aureus and recognizes the sequence motif NNGRR (R=A or G) (SEQ ID NO: 7), NNGRRN (R=A or G) (SEQ ID NO: 8), NNGRRT (R=A or G) (SEQ ID NO: 9), or NNGRRV (R=A or G) (V=A or G or C) (SEQ ID NO: 10). In the aforementioned embodiments, N can be any nucleotide residue, for example, any of A, G, C, or T. In some embodiments, the PAM sequence comprises ATTCCT (SEQ ID NO: 15).

Additionally or alternatively, a nucleic acid encoding a Cas9 molecule or Cas9 polypeptide may comprise a nuclear localization sequence (NLS). Nuclear localization sequences are known in the art, for example, SV40 NLS (Pro-Lys-Lys-Lys-Arg-Lys-Val; SEQ ID NO: 61.

In some embodiments, the at least one Cas9 molecule is a mutant Cas9 molecule. The Cas9 protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A, N854A, N863A and/or D986A. A S. pyogenes Cas9 protein with the D10A mutation may comprise an amino acid sequence of SEQ ID NO: 131. A S. pyogenes Cas9 protein with D10A and H849A mutations may comprise an amino acid sequence of SEQ ID NO: 132. Exemplary mutations with reference to the S. aureus Cas9 sequence to inactivate the nuclease activity include D10A and N580A. In certain embodiments, the mutant S. aureus Cas9 molecule comprises a D10A mutation. The nucleotide sequence encoding this mutant S. aureus Cas9 is set forth in SEQ ID NO: 133. In certain embodiments, the mutant S. aureus Cas9 molecule comprises a N580A mutation. The nucleotide sequence encoding this mutant S. aureus Cas9 molecule is set forth in SEQ ID NO: 134.

In some embodiments, the Cas9 protein is a VQR variant. The VQR variant of Cas9 is a mutant with a different PAM recognition, as detailed in Kleinstiver, et al. (Nature 2015, 523, 481-485, incorporated herein by reference).

A polynucleotide encoding a Cas9 molecule can be a synthetic polynucleotide. For example, the synthetic polynucleotide can be chemically modified. The synthetic polynucleotide can be codon optimized, for example, at least one non-common codon or less-common codon has been replaced by a common codon. For example, the synthetic polynucleotide can direct the synthesis of an optimized messenger mRNA, for example, optimized for expression in a mammalian expression system, as described herein. An exemplary codon optimized nucleic acid sequence encoding a Cas9 molecule of S. pyogenes is set forth in SEQ ID NO: 20. Exemplary codon optimized nucleic acid sequences encoding a Cas9 molecule of S. aureus, and optionally containing nuclear localization sequences (NLSs), are set forth in SEQ ID NOs: 21-27. Another exemplary codon optimized nucleic acid sequence encoding a Cas9 molecule of S. aureus comprises the nucleotides 1293-4451 of SEQ ID NO: 28.

b. Cas Fusion Protein

Alternatively or additionally, the CRISPR/Cas9-based gene editing system can include a fusion protein. The fusion protein can comprise two heterologous polypeptide domains. The first polypeptide domain comprises a Cas9 protein or a mutant Cas9 protein. The first polypeptide domain is fused to at least one second polypeptide domain. The second polypeptide domain has a different activity that what is endogenous to Cas9 protein. For example, the second polypeptide domain may have an activity such as transcription activation activity, transcription repression activity, transcription release factor activity, histone modification activity, nuclease activity, nucleic acid association activity, methylase activity, demethylase activity, acetylation activity, and/or deacetylation activity. The activity of the second polypeptide domain may be direct or indirect. The second polypeptide domain may have this activity itself (direct), or it may recruit and/or interact with a polypeptide domain that has this activity (indirect). The second polypeptide domain may be at the C-terminal end of the first polypeptide domain, or at the N-terminal end of the first polypeptide domain, or a combination thereof. The fusion protein may include one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three) second polypeptide domains in tandem.

The linkage from the first polypeptide domain to the second polypeptide domain can be through reversible or irreversible covalent linkage or through a non-covalent linkage, as long as the linker does not interfere with the function of the second polypeptide domain. For example, a Cas polypeptide can be linked to a second polypeptide domain as part of a fusion protein. As another example, they can be linked through reversible non-covalent interactions such as avidin (or streptavidin)-biotin interaction, histidine-divalent metal ion interaction (such as, Ni, Co, Cu, Fe), interactions between multimerization (such as, dimerization) domains, or glutathione S-transferase (GST)-glutathione interaction. As yet another example, they can be linked covalently but reversibly with linkers such as dibromomaleimide (DBM) or amino-thiol conjugation.

In some embodiments, the fusion protein includes at least one linker. A linker may be included anywhere in the polypeptide sequence of the fusion protein, for example, between the first and second polypeptide domains. A linker may be of any length and design to promote or restrict the mobility of components in the fusion protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may include, for example, a GS linker (Gly-Gly-Gly-Gly-Ser)n, wherein n is an integer between 0 and 10 (SEQ ID NO: 135). In a GS linker, n can be adjusted to optimize the linker length and achieve appropriate separation of the functional domains. Other examples of linkers may include, for example, Gly-Gly-Gly-Gly-Gly (SEQ ID NO: 136), Gly-Gly-Ala-Gly-Gly (SEQ ID NO: 137), Gly/Ser rich linkers such as Gly-Gly-Gly-Gly-Ser-Ser-Ser (SEQ ID NO: 138), or Gly/Ala rich linkers such as Gly-Gly-Gly-Gly-Ala-Ala-Ala (SEQ ID NO: 139).

In some embodiments, the second polypeptide domain has nuclease activity. A second polypeptide domain having nuclease activity may comprise, for example, FokI or TevI.

i) Transcription Activation Activity

The second polypeptide domain can have transcription activation activity, for example, a transactivation domain. For example, gene expression of endogenous mammalian genes, such as human genes, can be achieved by targeting a fusion protein of a first polypeptide domain, such as dCas9, and a transactivation domain to mammalian promoters via combinations of gRNAs. The transactivation domain can include a VP16 protein, multiple VP16 proteins, such as a VP48 domain or VP64 domain, p65 domain of NF kappa B transcription activator activity, TET1, VPR, VPH, Rta, and/or p300. For example, the fusion protein may comprise dCas9-p300. In some embodiments, p300 comprises a polypeptide having the amino acid sequence of SEQ ID NO: 140 or SEQ ID NO: 141. In other embodiments, the fusion protein comprises dCas9-VP64. In other embodiments, the fusion protein comprises VP64-dCas9-VP64. VP64-dCas9-VP64 may comprise a polypeptide having the amino acid sequence of SEQ ID NO: 142, encoded by the polynucleotide of SEQ ID NO: 143. VPH may comprise a polypeptide having the amino acid sequence of SEQ ID NO: 144, encoded by the polynucleotide of SEQ ID NO: 145. VPR may comprise a polypeptide having the amino acid sequence of SEQ ID NO: 146, encoded by the polynucleotide of SEQ ID NO: 147.

ii) Transcription Repression Activity

The second polypeptide domain can have transcription repression activity. Non-limiting examples of repressors include Kruppel associated box activity such as a KRAB domain or KRAB, MECP2, EED, ERF repressor domain (ERD), Mad mSIN3 interaction domain (SID) or Mad-SID repressor domain, SID4× repressor domain, MxiI repressor domain, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, and/or a domain having TATA box binding protein activity, or a combination thereof. In some embodiments, the second polypeptide domain has a KRAB domain activity, ERF repressor domain activity, MxiI repressor domain activity, SID4× repressor domain activity, Mad-SID repressor domain activity, DNMT3A or DNMT3L or fusion thereof activity, LSD1 histone demethylase activity, or TATA box binding protein activity. In some embodiments, the polypeptide domain comprises KRAB. For example, the fusion protein may be S. pyogenes dCas9-KRAB (polynucleotide sequence SEQ ID NO: 148; protein sequence SEQ ID NO: 149). The fusion protein may be S. aureus dCas9-KRAB (polynucleotide sequence SEQ ID NO: 150; protein sequence SEQ ID NO: 151).

iii) Transcription Release Factor Activity

The second polypeptide domain can have transcription release factor activity. The second polypeptide domain can have eukaryotic release factor 1 (ERF1) activity or eukaryotic release factor 3 (ERF3) activity.

iv) Histone Modification Activity

The second polypeptide domain can have histone modification activity. The second polypeptide domain can have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300. In some embodiments, p300 comprises a polypeptide of SEQ ID NO: 140 or SEQ ID NO: 141.

v) Nuclease Activity

The second polypeptide domain can have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease. A second polypeptide domain having nuclease activity may comprise, for example, FokI and/or TevI.

vi) Nucleic Acid Association Activity

The second polypeptide domain can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-turn-helix region, leucine zipper region, winged helix region, winged helix-turn-helix region, helix-loop-helix region, immunoglobulin fold, B3 domain, Zinc finger, HMG-box, Wor3 domain, and TAL effector DNA-binding domain.

vii) Methylase Activity

The second polypeptide domain can have methylase activity, which involves transferring a methyl group to DNA, RNA, protein, small molecule, cytosine, or adenine. In some embodiments, the second polypeptide domain includes a DNA methyltransferase.

viii) Demethylase Activity

The second polypeptide domain can have demethylase activity. The second polypeptide domain can include an enzyme that removes methyl (CH3-) groups from nucleic acids, proteins (in particular histones), and other molecules. Alternatively, the second polypeptide can convert the methyl group to hydroxymethylcytosine in a mechanism for demethylating DNA. The second polypeptide can catalyze this reaction. For example, the second polypeptide that catalyzes this reaction can be Tet1, also known as Tet1CD (Ten-eleven translocation methylcytosine dioxygenase 1; polynucleotide sequence SEQ ID NO: 152; amino acid sequence SEQ ID NO: 153). In some embodiments, the second polypeptide domain has histone demethylase activity. In some embodiments, the second polypeptide domain has DNA demethylase activity.

c. Guide RNA (gRNA)

The CRISPR/Cas-based gene editing system includes at least one gRNA molecule. For example, the CRISPR/Cas-based gene editing system may include two gRNA molecules. The at least one gRNA molecule can bind and recognize a target region. The gRNA provides the targeting of a CRISPR/Cas9-based gene editing system. The gRNA is a fusion of two noncoding RNAs: a crRNA and a tracrRNA. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to bind, and in some cases, cleave the target nucleic acid. The gRNA may target any desired DNA sequence by exchanging the sequence encoding a protospacer which confers targeting specificity through complementary base pairing with the desired DNA target. The CRISPR/Cas9-based gene editing system may include at least one gRNA, wherein the gRNAs target different DNA sequences. The target DNA sequences may be overlapping. The “target region” or “target sequence” or “protospacer” refers to the region of the target gene to which the CRISPR/Cas9-based gene editing system targets and binds or hybridizes to. The portion of the gRNA that targets the target sequence in the genome may be referred to as the “targeting sequence” or “targeting portion” or “targeting domain.” The CRISPR/Cas9-based gene editing system may include at least one gRNA, wherein the gRNAs target or hybridize to different DNA sequences. The target DNA sequences may be overlapping. The gRNA may comprise at its 5′ end the targeting domain that is sufficiently complementary to the target region to be able to hybridize to, for example, about 10 to about 20 nucleotides of the target region of the target gene, when it is followed by an appropriate Protospacer Adjacent Motif (PAM). The target sequence or protospacer is followed by a PAM sequence at the 3′ end of the target sequence or protospacer in the genome. Different Type II systems have differing PAM requirements, as detailed above.

“Protospacer” or “gRNA spacer” may refer to the region of the target sequence to which the CRISPR/Cas9-based gene editing system targets and binds or hybridizes; “protospacer” or “gRNA spacer” may also refer to the portion of the gRNA that is complementary to the targeted sequence in the genome. The protospacer may be, for example, 18 nucleotides or base pairs, 19 nucleotides or base pairs, 20 nucleotides or base pairs, 21 nucleotides or base pairs, 22 nucleotides or base pairs, 23 nucleotides or base pairs, 24 nucleotides or base pairs, 25 nucleotides or base pairs, 26 nucleotides or base pairs, or 27 nucleotides or base pairs in length.

The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The constant region of the gRNA may include the sequence of SEQ ID NO: 63 (RNA), which is encoded by a sequence comprising SEQ ID NO: 62 (DNA).

The targeting domain of the gRNA does not need to be perfectly complementary to the target region of the target DNA. In some embodiments, the targeting domain of the gRNA is at least 80%, 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19, or 20 nucleotides. For example, the DNA-targeting domain of the gRNA may be at least 80% complementary over at least 18 nucleotides of the target region. The target region may be on either strand of the target DNA.

The gRNA may target and bind or hybridize to a region or fragment of the dystrophin gene. The gRNA may target and bind or hybridize to a region or fragment of a mutant dystrophin gene. The gRNA may target and bind or hybridize to a region or fragment of a wild-type dystrophin gene. The gRNA may target an intron. The gRNA may target an intron that is juxtaposed with or adjacent to an exon of the dystrophin gene. The gRNA may target an intron that is juxtaposed with or adjacent to an exon of a mutant dystrophin gene. A fragment may be about 5 to about 200, about 10 to about 200, about 5 to about 300, or about 10 to about 300 nucleotides in length. A fragment may be at least about 5, at least about 10, at least about 15, at least about 20, at least about 30, at least about 40, at least about 50, or at least about 100 nucleotides in length. gRNA may target a fragment or portion of the dystrophin gene that comprises a mutation or deletion, or a sequence proximal or adjacent to or juxtapositioned thereto. In some embodiments, the gRNA targets intron 51. Intron 51 of the human dystrophin gene may comprise a polynucleotide sequence of SEQ ID NO: 128. In some embodiments, the gRNA targets intron 44. Intron 44 of the human dystrophin gene may comprise a polynucleotide sequence of SEQ ID NO: 156.

The gRNA may target and/or bind to and/or hybridize to a polynucleotide sequence comprising at least one of SEQ ID NOs: 29-51 and 87, or a complement thereof, or a variant thereof, or a truncation thereof. The gRNA may be encoded by a polynucleotide sequence comprising at least one of SEQ ID NOs: 29-51 and 87, or a complement thereof, or a variant thereof, or a truncation thereof. The gRNA may comprise a polynucleotide sequence comprising at least one of SEQ ID NOs: 29-51 and 87, or a complement thereof, or a variant thereof, or a truncation thereof. The gRNA may comprise a polynucleotide sequence comprising at least one of SEQ ID NOs: 64-86, 88, or a complement thereof, or a variant thereof, or a truncation thereof. The gRNA spacer may comprise a polynucleotide sequence comprising at least one of SEQ ID NOs: 29-51, 87, or a complement thereof, or a variant thereof, or a truncation thereof. The gRNA spacer may be encoded by a polynucleotide sequence comprising at least one of SEQ ID NOs: 29-51, 87, or a complement thereof, or a variant thereof, or a truncation thereof. A truncation may be 1, 2, 3, 4, 5, 6, 7, 8, or 9 nucleotides shorter than the reference sequence. In some embodiments, the gRNA scaffold is encoded by the polynucleotide sequence of SEQ ID NO: 52, or a complement thereof.

The gRNA may target and/or bind to and/or hybridize to a polynucleotide sequence comprising at least one of SEQ ID NOs: 157-170, or a complement thereof, or a variant thereof, or a truncation thereof. The gRNA may be encoded by a polynucleotide sequence comprising at least one of SEQ ID NOs: 157-170, or a complement thereof, or a variant thereof, or a truncation thereof. The gRNA may comprise a polynucleotide sequence comprising at least one of SEQ ID NOs: 171-184, or a complement thereof, or a variant thereof, or a truncation thereof. A truncation may be 1, 2, 3, 4, 5, 6, 7, 8, or 9 nucleotides shorter than the reference sequence.

As described above, the gRNA molecule comprises a targeting domain (also referred to as targeting sequence), which is a polynucleotide sequence complementary to the target DNA sequence. The gRNA may comprise a “G” at the 5′ end of the targeting domain or complementary polynucleotide sequence. The CRISPR/Cas9-based gene editing system may use gRNAs of varying sequences and lengths. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The number of gRNA molecules that may be included in the CRISPR/Cas9-based gene editing system can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules that may be included in the CRISPR/Cas9-based gene editing system can be less than 50 different gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs, less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, less than 3 different gRNAs, or less than 2 different gRNAs. The number of gRNAs that may be included in the CRISPR/Cas9-based gene editing system can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

d. Donor Sequence

The CRISPR/Cas9-based gene editing system may include at least one donor sequence. A donor sequence may comprise a fragment of a dystrophin gene. A donor sequence may comprise a fragment of a wild-type dystrophin gene. For example, a donor sequence may comprise a nucleic acid sequence encoding an exon or any combination of exons of the dystrophin gene. The donor sequence may comprise an exon of the wild-type dystrophin gene or a functional equivalent thereof. The donor sequence may comprise one or more exons of the wild-type dystrophin gene or a functional equivalent thereof. The donor sequence may comprise one or more exons and/or introns of the wild-type dystrophin gene or a functional equivalent thereof. The donor sequence may comprise one or more exons of the wild-type dystrophin gene selected from exon 52, 53, 54, 55, 56, 57, 58, 59, 60, 61, 62, 63, 64, 65, 66, 67, 68, 69, 70, 71, 72, 73, 74, 75, 76, 77, 78, and 79, or a combination thereof, or a functional equivalent thereof. In some embodiments, the donor sequence comprises exon 52. In some embodiments, the donor sequence includes exons 52-79. In some embodiments, the donor sequence includes exons 45-79. In some embodiments, exons 52-79 is referred to as a super exon. In some embodiments, exons 45-79 is referred to as a super exon. The donor sequence may further include at least one additional polynucleotide corresponding to intron sequences surrounding or near the exon(s) to be inserted. The donor sequence may further include at least one additional polynucleotide corresponding to intron sequences surrounding or near exon 52. The donor sequence may comprise a polynucleotide sequence selected from SEQ ID NOs: 53-56 and 154-155. In some embodiments, the donor sequence includes exons 52-79 and the donor sequence comprises a polynucleotide sequence selected from SEQ ID NOs: 53-56. In some embodiments, the donor sequence includes exons 45-79 and the donor sequence comprises a polynucleotide sequence selected from SEQ ID NOs: 154-155.

The donor sequence may be flanked on both sides by a gRNA spacer and/or a PAM sequence. The donor sequence may be flanked on the 5′-end and the 3′-end by a gRNA spacer and/or a PAM sequence. The gRNA spacer and/or a PAM sequence that flank the donor sequence directs the Cas9 protein to cut or excise the donor fragment from the CRISPR/Cas9-based gene editing system. This may thereby liberate the donor sequence for insertion into the genome. In some embodiments, the targeting region of the gRNA is complementary to the gRNA spacer that flanks the donor sequence. The gRNA spacer may comprise or be encoded by a polynucleotide selected from SEQ ID NO: 29-51 and 87 and 157-170.

The gRNA and donor sequence may be present in a variety of molar ratios. The molar ratio between the gRNA and donor sequence may be 1:1, or 1:5, or from 5:1 to 1:10, or from 1:1 to 1:5. The molar ratio between the gRNA and donor sequence may be at least 1:1, at least 1:2, at least 1:3, at least 1:4, at least 1:5, at least 1:6, at least 1:7, at least 1:8, at least 1:9, at least 1:10, at least 1:15, or at least 1:20. The molar ratio between the gRNA and donor sequence may be less than 20:1, less than 15:1, less than 10:1, less than 9:1, less than 8:1, less than 7:1, less than 6:1, less than 5:1, less than 4:1, less than 3:1, less than 2:1, or less than 1:1.

e. Repair Pathways

The CRISPR/Cas9-based gene editing system may be used to introduce site-specific double strand breaks at targeted genomic loci, such as an intron or exon of a dystrophin gene. Site-specific double-strand breaks are created when the CRISPR/Cas9-based gene editing system binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

i) Homology-Directed Repair (HDR)

Restoration of protein expression from a gene may involve homology-directed repair (HDR). A donor template may be administered to a cell. The donor template may include a nucleotide sequence encoding a full-functional protein or a partially functional protein. In such embodiments, the donor template may include fully functional gene construct for restoring a mutant gene, or a fragment of the gene that after homology-directed repair, leads to restoration of the mutant gene. In other embodiments, the donor template may include a nucleotide sequence encoding a mutated version of an inhibitory regulatory element of a gene. Mutations may include, for example, nucleotide substitutions, insertions, deletions, or a combination thereof. In such embodiments, introduced mutation(s) into the inhibitory regulatory element of the gene may reduce the transcription of or binding to the inhibitory regulatory element.

ii) NHEJ

Restoration of protein expression from gene may be through template-free NHEJ-mediated DNA repair. In certain embodiments, NHEJ is a nuclease mediated NHEJ, which in certain embodiments, refers to NHEJ that is initiated a Cas9 molecule that cuts double stranded DNA. The method comprises administering a presently disclosed CRISPR/Cas9-based gene editing system or a composition comprising thereof to a subject for gene editing.

Nuclease mediated NHEJ may correct a mutated target gene and offer several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment.

### 4. GENETIC CONSTRUCTS

The CRISPR/Cas9-based gene editing system may be encoded by or comprised within one or more genetic constructs. The CRISPR/Cas9-based gene editing system may comprise one or more genetic constructs. The genetic construct, such as a plasmid or expression vector, may comprise a nucleic acid that encodes the CRISPR/Cas9-based gene editing system and/or at least one of the gRNAs and/or a donor sequence. In certain embodiments, a genetic construct encodes one gRNA molecule, i.e., a first gRNA molecule, and optionally a Cas9 molecule or fusion protein. In some embodiments, a genetic construct encodes two gRNA molecules, i.e., a first gRNA molecule and a second gRNA molecule, and optionally a Cas9 molecule or fusion protein. In some embodiments, a first genetic construct encodes one gRNA molecule, i.e., a first gRNA molecule, and optionally a Cas9 molecule or fusion protein, and a second genetic construct encodes one gRNA molecule, i.e., a second gRNA molecule, and optionally a Cas9 molecule or fusion protein. In some embodiments, a first genetic construct encodes one gRNA molecule and one donor sequence, and a second genetic construct encodes a Cas9 molecule or fusion protein. In some embodiments, a first genetic construct encodes one gRNA molecule and a Cas9 molecule or fusion protein, and a second genetic construct encodes one donor sequence.

Genetic constructs may include polynucleotides such as vectors and plasmids. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual, Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise heterologous nucleic acid encoding the CRISPR/Cas-based gene editing system and may further comprise an initiation codon, which may be upstream of the CRISPR/Cas-based gene editing system coding sequence, and a stop codon, which may be downstream of the CRISPR/Cas-based gene editing system coding sequence. The genetic construct may include more than one stop codon, which may be downstream of the CRISPR/Cas-based gene editing system coding sequence. In some embodiments, the genetic construct includes 1, 2, 3, 4, or 5 stop codons. In some embodiments, the genetic construct includes 1, 2, 3, 4, or 5 stop codons downstream of the sequence encoding the donor sequence. A stop codon may be in-frame with a coding sequence in the CRISPR/Cas-based gene editing system. For example, one or more stop codons may be in-frame with the donor sequence. The genetic construct may include one or more stop codons that are out of frame of a coding sequence in the CRISPR/Cas-based gene editing system. For example, one stop codon may be in-frame with the donor sequence, and two other stop codons may be included that are in the other two possible reading frames. A genetic construct may include a stop codon for all three potential reading frames. The initiation and termination codon may be in frame with the CRISPR/Cas-based gene editing system coding sequence.

The vector may also comprise a promoter that is operably linked to the CRISPR/Cas-based gene editing system coding sequence. The promoter may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. The tissue specific promoter may be a skin specific promoter. The CRISPR/Cas-based gene editing system may be under the light-inducible or chemically inducible control to enable the dynamic control of gene/genome editing in space and time. The promoter operably linked to the CRISPR/Cas-based gene editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. Examples of a tissue specific promoter, such as a muscle or skin specific promoter, natural or synthetic, are described in U.S. Patent Application Publication No. US20040175727, the contents of which are incorporated herein in its entirety. The promoter may be a CK8 promoter, a Spc512 promoter, a MHCK7 promoter, for example.

The genetic construct may also comprise a polyadenylation signal, which may be downstream of the CRISPR/Cas-based gene editing system. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, Calif.).

Coding sequences in the genetic construct may be optimized for stability and high levels of expression. In some instances, codons are selected to reduce secondary structure formation of the RNA such as that formed due to intramolecular bonding.

The genetic construct may also comprise an enhancer upstream of the CRISPR/Cas-based gene editing system or gRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as green fluorescent protein (“GFP”) and/or a selectable marker, such as hygromycin (“Hygro”).

The genetic construct may be useful for transfecting cells with nucleic acid encoding the CRISPR/Cas-based gene editing system, which the transformed host cell is cultured and maintained under conditions wherein expression of the CRISPR/Cas-based gene editing system takes place. The genetic construct may be transformed or transduced into a cell. The genetic construct may be formulated into any suitable type of delivery vehicle including, for example, a viral vector, lentiviral expression, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a system or component thereof as detailed herein. Suitable cell types are detailed herein. In some embodiments, the cell is a stem cell. The stem cell may be a human stem cell. In some embodiments, the cell is an embryonic stem cell. The stem cell may be a human pluripotent stem cell (iPSCs). Further provided are stem cell-derived neurons, such as neurons derived from iPSCs transformed or transduced with a DNA targeting system or component thereof as detailed herein.

a. Viral Vectors

A genetic construct may be a viral vector. Further provided herein is a viral delivery system. Viral delivery systems may include, for example, lentivirus, retrovirus, adenovirus, mRNA electroporation, or nanoparticles. In some embodiments, the vector is a modified lentiviral vector. In some embodiments, the viral vector is an adeno-associated virus (AAV) vector. The AAV vector is a small virus belonging to the genus Dependovirus of the Parvoviridae family that infects humans and some other primate species.

AAV vectors may be used to deliver CRISPR/Cas9-based gene editing systems using various construct configurations. For example, AAV vectors may deliver Cas9 or fusion protein and gRNA expression cassettes on separate vectors or on the same vector. Alternatively, if the small Cas9 proteins or fusion proteins, derived from species such as Staphylococcus aureus or Neisseria meningitidis, are used then both the Cas9 and up to two gRNA expression cassettes may be combined in a single AAV vector. In some embodiments, the AAV vector has a 4.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biol. Chem. 2013, 288, 28814-28823).

The genetic construct may comprise a polynucleotide sequence selected from SEQ ID NOs: 57-60. The genetic construct may comprise a polynucleotide sequence selected from SEQ ID NOs: 29-51, 53-56, 87, 154-155, 157-169, and 170, or a complement thereof, or a fragment thereof.

### 5. PHARMACEUTICAL COMPOSITIONS

Further provided herein are pharmaceutical compositions comprising the above-described genetic constructs or gene editing systems. In some embodiments, the pharmaceutical composition may comprise about 1 ng to about 10 mg of DNA encoding the CRISPR/Cas-based gene editing system. The systems or genetic constructs as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition for gene editing in skeletal muscle or cardiac muscle at a concentration less than 6 mg/mL.

### 6. ADMINISTRATION

The systems or genetic constructs as detailed herein, or at least one component thereof, may be administered or delivered to a cell. Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. The system, genetic construct, or composition comprising the same, may be electroporated using BioRad Gene Pulser Xcell or Amaxa Nucleofector IIb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The systems or genetic constructs as detailed herein, or at least one component thereof, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the system, genetic construct, or composition comprising the same, is administered to a subject intramuscularly, intravenously, or a combination thereof. The systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the brain or other component of the central nervous system. The composition may be injected into the skeletal muscle or cardiac muscle. For example, the composition may be injected into the tibialis anterior muscle or tail. For veterinary use, the systems, genetic constructs, or compositions comprising the same may be administered as a suitably acceptable formulation in accordance with normal veterinary practice. The veterinarian may readily determine the dosing regimen and route of administration that is most appropriate for a particular animal. The systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound. Alternatively, transient in vivo delivery of CRISPR/Cas-based systems by non-viral or non-integrating viral gene transfer, or by direct delivery of purified proteins and gRNAs containing cell-penetrating motifs may enable highly specific correction and/or restoration in situ with minimal or no risk of exogenous DNA integration.

Upon delivery of the presently disclosed systems or genetic constructs as detailed herein, or at least one component thereof, or the pharmaceutical compositions comprising the same, and thereupon the vector into the cells of the subject, the transfected cells may express the gRNA molecule(s) and the Cas9 molecule or fusion protein.

a. Cell Types

Any of the delivery methods and/or routes of administration detailed herein can be utilized with a myriad of cell types. Further provided herein is a cell transformed or transduced with a system or component thereof as detailed herein. For example, provided herein is a cell comprising an isolated polynucleotide encoding a CRISPR/Cas9 system as detailed herein. Suitable cell types are detailed herein, for example, those cell types currently under investigation for cell-based therapies, including, but not limited to, immortalized myoblast cells, such as wild-type and DMD patient derived lines, primal DMD dermal fibroblasts, stem cells such as induced pluripotent stem cells, embryonic stem cell, hematopoietic stem cell, bone marrow-derived progenitors, skeletal muscle progenitors, human skeletal myoblasts from DMD patients, CD 133+ cells, mesoangioblasts, cardiomyocytes, hepatocytes, chondrocytes, mesenchymal progenitor cells, hematopoietic stem cells, smooth muscle cells, and MyoD- or Pax7-transduced cells, or other myogenic progenitor cells. The cell may be a human stem cell. The stem cell may be a human induced pluripotent stem cell (iPSC). The cell may be a muscle cell. Immortalization of human myogenic cells can be used for clonal derivation of genetically corrected myogenic cells. Cells can be modified ex vivo to isolate and expand clonal populations of immortalized DMD myoblasts that include a genetically corrected or restored dystrophin gene and are free of other nuclease-introduced mutations in protein coding regions of the genome.

### 7. KITS

Provided herein is a kit, which may be used to correct a mutated dystrophin gene and/or restore dystrophin function. The kit comprises genetic constructs or a composition comprising the same, for restoring dystrophin function, as described above, and instructions for using said composition. In some embodiments, the kit comprises at least one gRNA comprising or hybridizing to or targeting or encoded by a polynucleotide sequence selected from SEQ ID NOs: 29-51, 87, 157-170, a complement thereof, a variant thereof, or fragment thereof, and/or at least one gRNA spacer comprising or encoded by a polynucleotide sequence selected from SEQ ID NOs: 29-51, 87, 157-170, a complement thereof, a variant thereof, or fragment thereof, and/or at least one gRNA comprising a polynucleotide sequence selected from SEQ ID NOs: 64-86, 88, 171-184, a complement thereof, a variant thereof, or fragment thereof, and/or a donor sequence comprising a polynucleotide sequence selected from SEQ ID NOs: 53-56, 154, and 155, a complement thereof, a variant thereof, or fragment thereof. The kit may further include instructions for using the CRISPR/Cas-based gene editing system.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The genetic constructs or a composition comprising thereof for restoring dystrophin function may include a modified AAV vector that includes a gRNA molecule(s) and a Cas9 protein or fusion protein, as described above, that specifically binds and cleaves a region of the dystrophin gene. The CRISPR/Cas-based gene editing system, as described above, may be included in the kit to specifically bind and target a particular region, for example, exon 52 or intron 51 or intron 44, in the gene.

### 8. METHODS

a. Methods for Restoring Dystrophin Function

The CRISPR/Cas9-based gene editing systems provided herein may be used for restoring dystrophin function. The CRISPR/Cas9-based gene editing systems may restore dystrophin function by adding one or more exons to restore the reading frame of dystrophin. Use of the presently disclosed CRISPR/Cas9-based gene editing systems delivered to a target muscle, for example, may restore the expression of a full-functional or partially-functional protein with a repair template or donor DNA, which can replace the entire gene or the region containing the mutation.

Provided herein are methods of restoring dystrophin function. The methods may be used for restoring dystrophin function in a cell or a subject having a mutant dystrophin gene. The methods may include contacting the cell or the subject with a system as detailed herein, a recombinant polynucleotide as detailed herein, or a vector as detailed herein. In some embodiments, dystrophin function is restored by insertion of the donor sequence, for example, insertion of exons 52-79 or exons 45-79 of the wild-type dystrophin gene. In some embodiments, the subject is suffering from Duchenne Muscular Dystrophy.

The methods may be used for restoring dystrophin function in a cell or a subject having a disrupted dystrophin gene caused by one or more deleted or mutated exons. The methods may include contacting the cell or the subject with a system as detailed herein, a recombinant polynucleotide as detailed herein, or a vector as detailed herein. In some embodiments, dystrophin function is restored by inserting one or more wild-type exons of dystrophin gene corresponding to the one or more deleted or mutated exons. In some embodiments, dystrophin function is restored by insertion of the donor sequence, for example, insertion of exons 52-79 or exons 45-79 of the wild-type dystrophin gene. In some embodiments, the subject is suffering from Duchenne Muscular Dystrophy.

### 9. EXAMPLES

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Materials and Methods

Plasmid Design and AAV Production. The ITR-containing Staphylococcus aureus Cas9 (pAAV-SaCas9) expression plasmid was generated by adding a 3×HA epitope to the carboxyl-terminus of SaCas9 using Gibson cloning strategies. The CMV-SaCas9-3×HA-polyA was transferred to a new plasmid (pSaCas9) without ITRs for stability in cell culture experiments. A separate plasmid with a hU6-driven guide RNA cassette (Nelson, C. E., et al. Science 2016, 351, 403-407) (pU6-gRNA) was used with BbsI cloning to screen guides in vitro. For AAV-gRNA-donor plasmids (pAAV-g12-Ex52, pAAV-g7-Ex52, and pAAv-g7-Superexon), gene blocks were synthesized by Integrated DNA technology (IDT) and integrated into ITR-containing plasmids by Gibson cloning strategies. Intact ITRs were verified by SmaI digest before AAV production on all vectors. Multiple batches of AAV2 and AAV9 were produced at Duke University. Titers were measured by qPCR with a plasmid standard curve.

In Vitro gRNA Screening. A panel of gRNAs (TABLE 1) were designed to target intron 51 of the human DMD gene and compared for SaCas9 activity by Surveyor assay in HEK293T cells and DMD patient myoblasts. HEK293T cells were maintained in Dulbecco's Modified Eagle's Medium (DMEM, Invitrogen) with 10% Fetal Bovine Serum (FBS, Sigma) and 1% penicillin-streptomycin (P/S, Gibco). Immortalized DMD patient 8036 myoblasts (DM8036 cell line with a deletion of exons 48-50 in the DMD gene)(Mamchaoui, K., et al. Skelet. Muscle 2011, 1, 34) were maintained in skeletal muscle media (PromoCell) with 20% FBS (Sigma), 50 μg/mL fetuin (Sigma), 10 ng/mL human epidermal growth factor (Sigma), 1 ng/mL human basic fibroblast growth factor (bFGF, Sigma), 10 μg/mL human insulin (Sigma), 400 ng/mL dexamethasone (Sigma), 1% GlutaMAX (Invitrogen), and 1% P/S. Cells were incubated at 37° C. with 5% CO₂. HEK293T cells were transfected with 375 ng pSaCas9 and 125 ng pU6-gRNA plasmid using Lipofectamine 2000 (Invitrogen) according to the manufacturer's protocol. DMD myoblasts were electroporated with 10 μg pSaCas9 and 10 μg pU6-gRNA plasmid with a Gene Pulser XCell (BioRAD) in PBS using previously optimized conditions (Ousterout, O. G., et al. Mol. Ther. 2015, 23, 523-532). Cells were incubated for 72 hours, and genomic DNA was isolated with a DNeasy kit (Qiagen). Indels were identified by PCR of the region of interest (Surveyor Primers provided in TABLE 2) performed using the Invitrogen AccuPrime High Fidelity PCR kit, following by incubation with the Surveyor Nuclease and electrophoresed on TBE gels (Life Technologies) as previously described (Nelson, C. E., et al. Science 2016, 351, 403-407; Guschin, D. Y., et al. Methods Mol. Biol. 2010, 649, 247-256).

TABLE 1
SaCas9 human DMD intron 51 target sequences.

| | Spacer Sequence | | |
|---|---|---|---|
| | (Sequence the gRNA targets | | |
| | and binds) | PAM | gRNA |
| gScbl | GCACTACCAGAGCTAACTCA | NNGRRT | GCACUACCAGAGCUAACUCA |
| | (SEQ ID NO: 87) | (SEQ ID | (SEQ ID NO: 88) |
| | | NO: 9) | |
| g1 | CTTTACTTTGTATTATGTAAA | AGGAAT | CUUUACUUUGUAUUAUGUAAA |
| | (SEQ ID NO: 29) | (SEQ ID | (SEQ ID NO: 64) |
| | | NO: 89) | |
| g2 | TTTGAAATATTTTTGATATCT | AAGAAT | UUUGAAAUAUUUUUGAUAUCU |
| | (SEQ ID NO: 30) | (SEQ ID | (SEQ ID NO: 65) |
| | | NO: 90) | |
| g3 | TTTAAGTAATCCGAGGTACTC | CGGAAT | UUUAAGUAAUCCGAGGUACUC |
| | (SEQ ID NO: 31) | (SEQ ID | (SEQ ID NO: 66) |
| | | NO: 91) | |
| g4 | TTTAAATACATTGTCGTAATT | CAGAAT | UUUAAAUACAUUGUCGUAAUU |
| | (SEQ ID NO: 32) | (SEQ ID | (SEQ ID NO: 67) |
| | | NO: 92) | |
| g5 | TACCTTAATTTTGACGTCACA | CAGAAT | UACCUUAAUUUUGACGUCACA |
| | (SEQ ID NO: 33) | (SEQ ID | (SEQ ID NO: 68) |
| | | NO: 92) | |
| g6 | ATTTGACAGGTGAGAAATCTC | AGGGGT | AUUUGACAGGUGAGAAAUCUC |
| | (SEQ ID NO: 34) | (SEQ ID | (SEQ ID NO: 69) |
| | | NO: 93) | |
| g7 | TCATTTATAATACAGGGGAAT | AGGAAT | UCAUUUAUAAUACAGGGGAAU |
| | (SEQ ID NO: 35) | (SEQ ID | (SEQ ID NO: 70) |
| | | NO: 89) | |
| g8 | TTAAAGTCATTTATAATACAG | GGGAAT | UUAAAGUCAUUUAUAAUACAG |
| | (SEQ ID NO: 36) | (SEQ ID | (SEQ ID NO: 71) |
| | | NO: 94) | |
| g9 | AAATAGACACTGAAGAAAGGG | AAGAAT | AAAUAGACACUGAAGAAAGGG |
| | (SEQ ID NO: 37) | (SEQ ID | (SEQ ID NO: 72) |
| | | NO: 90) | |
| g10 | CCCCAATTAAAATAAAATTTA | CTGAGT | CCCCAAUUAAAAUAAAAUUUA |
| | (SEQ ID NO: 38) | (SEQ ID | (SEQ ID NO: 73) |
| | | NO: 95) | |
| g11 | TAAGTAATCCGAGGTACTC | CGGAAT | UAAGUAAUCCGAGGUACUC |
| (g3) | (SEQ ID NO: 39) | (SEQ ID | (SEQ ID NO: 74) |
| | | NO: 91) | |
| g12 | TTAAGTAATCCGAGGTACTC | CGGAAT | UUAAGUAAUCCGAGGUACUC |
| (g3) | (SEQ ID NO: 40) | (SEQ ID | (SEQ ID NO: 75) |
| | | NO: 91) | |
| g13 | GTTTAAGTAATCCGAGGTACT | CGGAAT | GUUUAAGUAAUCCGAGGUAC |
| (g3) | C | (SEQ ID | UC |
| | (SEQ ID NO: 41) | NO: 91) | (SEQ ID NO: 76) |
| g14 | GGTTTAAGTAATCCGAGGTAC | CGGAAT | GGUUUAAGUAAUCCGAGGUA |
| (g3) | TC | (SEQ ID | CUC |
| | (SEQ ID NO: 42) | NO: 91) | (SEQ ID NO: 77) |
| g15 | TTGACAGGTGAGAAATCTC | AGGGGT | UUGACAGGUGAGAAAUCUC |
| (g6) | (SEQ ID NO: 43) | (SEQ ID | (SEQ ID NO: 78) |
| | | NO: 93) | |
| g16 | TTTGACAGGTGAGAAATCTC | AGGGGT | UUUGACAGGUGAGAAAUCUC |
| (g6) | (SEQ ID NO: 44) | (SEQ ID | (SEQ ID NO: 79) |
| | | NO: 93) | |
| g17 | CATTTGACAGGTGAGAAATCT | AGGGGT | CAUUUGACAGGUGAGAAAUCU |
| (g6) | C | (SEQ ID | C |
| | (SEQ ID NO: 45) | NO: 93) | (SEQ ID NO: 80) |
| g18 | TCATTTGACAGGTGAGAAATC | AGGGGT | UCAUUUGACAGGUGAGAAAUC |
| (g6) | TC | (SEQ ID | UC |
| | (SEQ ID NO: 46) | NO: 93) | (SEQ ID NO: 81) |
| g19 | ATTTATAATACAGGGGAAT | AGGAAT | AUUUAUAAUACAGGGGAAU |
| (g7) | (SEQ ID NO: 47) | (SEQ ID | (SEQ ID NO: 82) |
| | | NO: 89) | |
| g20 | CATTTATAATACAGGGGAAT | AGGAAT | CAUUUAUAAUACAGGGGAAU |
| (g7) | (SEQ ID NO: 48) | (SEQ ID | (SEQ ID NO: 83) |
| | | NO: 89) | |
| g21 | GTCATTTATAATACAGGGGAA | AGGAAT | GUCAUUUAUAAUACAGGGGAA |
| (g7) | T | (SEQ ID | U |
| | (SEQ ID NO: 49) | NO: 89) | (SEQ ID NO: 84) |
| g22 | AGTCATTTATAATACAGGGGA | AGGAAT | AGUCAUUUAUAAUACAGGGGA |
| (g7) | AT | (SEQ ID | AU |
| | (SEQ ID NO: 50) | NO: 89) | (SEQ ID NO: 85) |
| g23 | GCACTACCAGAGCTAACTCA | | GCACUACCAGAGCUAACUCA |
| | (SEQ ID NO: 51) | | (SEQ ID NO: 86) |

TABLE 2
Primer sequences.

| Description | Forward Primer (5′-3′) |
|---|---|
| Surveyor | Fwd: CTGATGCTCTCCAAACTTGCC (SEQ ID NO: 98) |
| (g1, g5, g6) | Rev: TGCTTTGTGTGTCCCATGCT (SEQ ID NO: 99) |
| Surveyor | Fwd: ATACCTCTGAGATTGTGGTCCT (SEQ ID NO: 100) |
| (g2, g3, g4) | Rev: TGGGCAGCGGTAATGAGTTC (SEQ ID NO: 101) |
| Surveyor | Fwd: TTACTGAGTTTTAGAACCAGAGCTA (SEQ ID NO: 102) |
| (g7, g8) | Rev: AGGGTTCTTCAGCGTTGTGT (SEQ ID NO: 103) |
| Surveyor | Fwd: AGCAGGAGTCAAAGTACAGAGT (SEQ ID NO: 104) |
| (g9, g10) | Rev: TCCGGAGTACCTCGGATTAC (SEQ ID NO: 105) |
| gDNA integration | Fwd: TTACTGAGTTTTAGAACCAGAGCTA (SEQ ID NO: 106) |
| PCR | Rev: GAGTTCTTCCAACTGGGGAC (SEQ ID NO: 107) |
| CDNA integration | Fwd: CTGACCACTATTGGAGCCTCTC (SEQ ID NO: 108) |
| PCR | Rev: GAGTTCTTCCAACTGGGGAC (SEQ ID NO: 109) |
| 3′ RACE GSP | GTAGTCGTTTAAACCGCTGATCAGCCTCG (SEQ ID NO: 110) |
| ddPCR - Corrected | Fwd: GCTTTCTCTGCTTGATCAAG (SEQ ID NO: 111) |
| (Ex51-Ex52 junction) | Rev: GAGTTCTTCCAACTGGGGAC (SEQ ID NO: 112) |
| | Probe: GCAACAATGCAGGATTTG (SEQ ID NO: 113) |
| ddPCR - Unedited | Fwd: GCTTTCTCTGCTTGATCAAG (SEQ ID NO: 114) |
| (Ex51-Ex53 junction) | Rev: CGGTTCTGAAGGTGTTCTTGTA (SEQ ID NO: 115) |
| | Probe: AGCAGAAGTTGAAAG (SEQ ID NO: 116) |
| ddPCR - | Fwd: GATGAGCTGGACCTCAAGCT (SEQ ID NO: 117) |
| Normalization | Rev: GTGGCTCACGTTCTCTTTCA (SEQ ID NO: 118) |
| (Ex59-Ex60 junction) | Probe: CGAGAAAGTCAAGGCACT (SEQ ID NO: 119) |
| Tn5-Top | CAAGCAGAAGACGGCATACGAGATNNNNNNNNNNGTCTCGTGGG |
| | CTCGGAGATGTGTATAAGAGACAG (SEQ ID NO: 120) |
| Tn5-Bottom | [Phos]CTGTCTCTTATACACATCT (SEQ ID NO: 121) |
| Tn5-GSP (g7) | AAGCAGTGGTATCAACGCAGAGTACCAGAGAAAATAGACACTGA |
| | AGAAAGGG (SEQ ID NO: 122) |
| Tn5-GSP (g12) | AAGCAGTGGTATCAACGCAGAGTACCCTTAATTTTGACGTCACAC |
| | AGAATG (SEQ ID NO: 123) |
| Tn5-Universal | CAAGCAGAAGACGGCATACGAGAT (SEQ ID NO: 124) |
| Tn5-BC, [i5 barcode] | AATGATACGGCGACCACCGAGATCTACAC[NNNNNN]CGGAAGCA |
| | GTGGTATCAACGCAGAGTAC (SEQ ID NO: 125) |
| Tn5-Read1 | CGGAAGCAGTGGTATCAACGCAGAGTAC (SEQ ID NO: 126) |
| (Miseq/Novaseq) | |
| Tn5-Index1 | GTACTCTGCGTTGATACCACTGCTTCCG (SEQ ID NO: 127) |
| (Novaseq) | |

Animals. All experiments involving animals were conducted with strict adherence to the guidelines for the care and use of laboratory animals of the National Institutes of Health (NIH). All experiments were approved by the Institutional Animal Care and Use Committee (IACUC) at Duke University. The hDMD/mdx mouse (t Hoen, P. A., et al. J. Biol. Chem. 2008, 283, 5899-5907) was kindly provided by Leiden University Medical Center. The generation of the hDMDΔ52/mdx mouse was completed by the Duke Transgenic Mouse Facility. Briefly, B6SJLF1/J donor females were superovulated by intraperitoneal injection of 5IU PMSG on day one and 5IU HCG on day three, followed by mating with fertile hDMD/mdx males. On day four, embryos were harvested and injected with mRNA encoding S. pyogenes Cas9 and gRNAs targeting human intron 51 (CTCTGATAACCCAGCTGTGTGTT, SEQ ID NO: 96) and human intron 52 (CTAGACCATTTCCCACCAGTTCT; SEQ ID NO: 97). Injected embryos were implanted into pseudo-pregnant CD1 female mice. Genomic DNA was extracted from ear punches of chimeric pups using the DNeasy Blood and Tissue Kit (Qiagen) and screened for presence or deletion of exon 52. Mice with loss of exon 52 were bred with mdx mice. The resulting male hDMDΔ52/mdx (het;hemi) mice were used for experiments.

In Vitro AAV Transductions. Primary myoblasts were isolated from the tibialis anterior (TA) and gastrocnemius muscles of hDMDΔ52/mdx mice, as previously described (Springer, M. L., Rando, T. A. & Blau, H. M. Gene delivery to muscle. Curr. Protoc. Hum. Genet. Chapter 13, Unit13.14), and maintained in F10 media (Invitrogen) supplemented with 20% FBS, 5 ng/mL bFGF, and 1% P/S. Cells were grown on plates coated with bovine type I collagen (Sigma) and incubated at 37° C. with 5% CO₂. For transductions, cells were plated for 1.5 hours then AAV2-SaCas9 and AAV2-gRNA-donor vectors were combined, added to the plates at an MOI of 1×10⁶ total vectors per cell. Cells were immediately spun at 3000×g for 5 min and returned to the incubator. Once cells reached 70% confluency, the media was changed to DMEM supplemented with 5% horse serum and 1% P/S and replaced every 2 days for differentiation into myofibers. Cells were differentiated for 10 days and processed for analysis of genomic DNA, total RNA, and protein as described.

Genomic DNA and RNA Analysis from Primary hDMDΔ52/mdx Myoblasts. Genomic DNA was isolated using the DNeasy kit (Qiagen) according to the manufacturer's protocol. Total RNA was isolated using QIAshredder and RNeasy Plus kits (Qiagen). First-strand cDNA synthesis was performed using 500 ng total RNA per sample using the SuperScript VILO Reverse Transcription Kit (Invitrogen) and incubated at 25° C. for 10 min, 42° C. for 2 hours, and 85° C. for 5 min. Donor integration was detected by PCR (Primers provided in TABLE 2) using the Invitrogen AccuPrime High Fidelity PCR kit according to the manufacturer's protocol and electrophoresed on 1% agarose gels. 3′ RACE was carried out on RNA samples using the SMARTer RACE 5′/3′ kit (Takara) for cDNA synthesis and primary PCR (Primers provided in TABLE 2) using Program 1 according to the manufacturer's instructions.

In Vivo AAV Administration. Adult 6-8 week-old mice were administered AAV by intramuscular injection into the tibialis anterior muscle with 40 μL PBS or AAV vector per mouse. For the donor comparative study, 1.56e12 total vg was administered to 1:1 treatment groups (7.81e11 AAV-Cas9 and 7.81e11 AAV-donor) and 2.13e12 total vg was administered to 1:5 treatment groups (3.55e11 AAV-Cas9 and 1.77e12 AAV-donor). For the gRNA comparative study, 8.64e11 total vg was administered to the 1:1 treatment groups (4.32e11 AAV-Cas9 and 4.32e11 AAV-donor) and 7.00e11 total vg was administered to the 1:5 treatment groups (1.17e11 AAV-Cas9 and 5.83e11 AAV-donor). Two-day-old (P2) neonatal mice were administered AAV by intravenous injection through the facial vein (Gombash Lampe, et al. J. Vis. Exp. 2014, e52037) with 40 μL AAV vector per mouse. For the 1:1 treatment groups, 8.64e11 total vg was administered (4.32e11 AAV-Cas9 and 4.32e11 AAV-donor) and for the 1:5 treatment groups, 7.00e11 total vg was administered (1.17e11 AAV-Cas9 and 5.83e11 AAV-donor). At set time points, mice were euthanized and skeletal muscle, cardiac muscle, and serum was collected.

Droplet Digital PCR. Quantitative ddPCR was performed on cDNA and gDNA samples using the WX200 Droplet Digital PCR system according to the manufacturer's instructions. To quantify corrected transcript levels, RNA was extracted from mouse tissues using the Qiagen Universal kit. Subsequently, First-strand cDNA synthesis was performed using 500 ng total RNA per sample as stated above. Corrected hDMD transcripts containing exon 52 were detected using the QX200 ddPCR Supermix for Probes without dUTP (BioRad) and Taqman assays with probes (TABLE 2) designed to bind to the human dystrophin Ex51-52 junction (corrected, ThermoFisher custom assay ID: AP2XDZ9), human dystrophin Ex51-53 junction (unedited, ThermoFisher custom assay ID: AP327K6), and human dystrophin Ex59-60 (input normalization, ThermoFisher custom assay ID: AP47Z63). Quantification was determined based on the number of positive droplets in each reaction using QuantaSoft Analysis software (BioRad). cDNA input for corrected or unedited transcript levels was normalized by dividing the number of Ex51-52 or Ex51-53 of positive droplets, respectively, by the number of positive Ex59-60 droplets in each reaction. The percentage of corrected transcripts was calculated as (Normalized Ex51-52)/[(Normalized Ex51-52)+(Normalized Ex51-53)]×100. For vector genome quantification, gDNA was extracted from mouse tissues using the Qiagen DNeasy kit and digested with HindIII-HF at 37° C. for 1 hour. Episomes were detected using the QX200 ddPCR Supermix for Probes without dUTP (BioRad) and BioRad assays with probes (TABLE 2) designed to bind SaCas9 (AAV-SaCas9, BioRad unique assay ID: dCNS159380965), U6 (AAV-gRNA-donor, BioRad unique assay ID: dCNS116676529), and mouse EEF2 (input normalization, BioRad unique assay ID: dMmuCNS781688813). Quantification was determined based on the number of positive droplets in each reaction using QuantaSoft Analysis software. Episome quantification was calculated as viral genomes per diploid genome (vg/dg) by dividing the number of SaCas9 or U6 positive droplets by the number of mouse EEF2 positive droplets in the corresponding reaction.

Transposon-mediated target enrichment and sequencing. Tn5 transposase protein was expressed and purified as previously described (Picelli, S., et al. Genome Res. 2014, 24, 2033-2040). Tagmentation of genomic DNA was completed as previously described (Giannoukos, G., et al. BMC Genomics 2018, 19, 212), with the following modifications to include unique molecular indexes (UMIs). For RNA transcript analysis, first-strand cDNA synthesis was performed using 500 ng total RNA per sample as stated above. Second-strand synthesis was performed using Kenow fragment DNA polymerase (NEB) and purified using Ampure beads (Beckman Coulter) at 1.8×. All primer sequences are provided in TABLE 2. In brief, the linker oligonucleotides (Tn5-Top contains Illumina i7 adapter sequence and 10 nucleotide UMI, Tn5-Bottom contains Tn5-ME sequence) were annealed and assembled on Tn5. Genomic DNA was quantified using NanoDrop (ThermoFisher) and second-strand products were quantified using Qubit Fluorometric Quantification (ThermoFisher). Tagmentation of 200 ng genomic DNA or second-strand products was performed using a 1:40 dilution of assembled Tn5 and purified using DNA Clean and Concentrator-5 columns or 96-well kits (Zymo). To enrich the targeted sequence, first round PCR using a genome specific primer (Tn5-GSP, contains custom adapter) was used with a reverse primer (Tn5-Universal) specific for the i7 adapter sequence inserted by the transposon for 25 cycles. Amplicons were purified with Ampure beads at 1.8×. Second round PCR using a barcode primer (Tn5-BC) specific for the custom adapter sequence was used to add 6-nucleotide experimental barcodes and the Illumina i5 adapter was used with the Tn5-Universal reverse primer for 15 cycles. Amplicons were gel-purified, followed by purification with Ampure beads at 0.6× to select for fragment sizes greater than 250 bp. Sequencing was conducted on an Illumina Miseq using 250/50-cycle paired-end reads with a custom read 1 primer (Tn5-Read1) or on an Illumina Novaseq v1.5 using 300-cycle single-end reads with a custom read 1 primer (Tn5-Read1) and custom index 1 primer (Tn5-Index1). The Tn5-based method is expected to reduce PCR-related bias from amplicon size; however, some bias may remain from the transposition selectivity (Giannoukos, G., et al. BMC Genomics 2018, 19, 212). Briefly, the analysis steps are as follows: Demultiplex. Demultiplex fastq files using the list of barcodes for each sample. Trim. Remove the 3′ adapters and low-quality bases using Trimmomatic. Alignment and deduplication. Using bwa-mem, align the reads to reference genomes (gDNA aligned to mouse genome (GRCm38)+human DMD; cDNA aligned to human dystrophin cDNA) with PCR duplicates marked using Picard MarkDuplicates and removed. Alignment to reference bin sequences. Build reference amplicons to align to the targeted locus and expected edits. To remove reads that are due to false priming, filter out reads that do not contain the 20 bases directly adjacent to the GSP expected sequence. To remove reads that do not extend far enough past the edit site, filter out reads that are shorter than the required minimum length for binning. Align on-site deduplicated reads to reference amplicons using bwa-mem. Identify reads where there is an indel ±15 bp at the expected cute site or junction. The number of distinct UMIs were counted for each edit.

Western blot. Protein was isolated from muscle tissues by disruption with a BioMasher II Micro Tissue Homogenizer (VWR) in RIPA buffer (Sigma) with a protease inhibitor cocktail (Roche) and incubated for 30 minutes on ice with intermittent vortexing. Samples were spun at 16,000×g at 4° C. for 30 minutes and supernatant was collected. Total protein was quantified using the BCA Protein assay kit (Pierce) according to the manufacturer's protocol and measured on a BioTek Synergy 2 Multi-Mode Microplate Reader. S ample was mixed with NuPAGE loading buffer (Invitrogen) and 5% β-mercaptoethanol, and 3.125 μg of hDMD/mdx protein or 25 μg of all other protein samples was heated at 100° C. for 10 min. Samples were loaded into 4-12% NuPAGE Bis-Tris gels (Invitrogen) with MES buffer (Invitrogen) and electrophoresed for 45 min at 200V on ice. Protein was transferred to nitrocellulose membranes for 90 minutes in 1× tris-glycine transfer buffer with 0.01% SDS at 4° C. at 400 mA. The blot was blocked in 5% milk-TBST (50 mM Tris, 150 mM NaCl and 0.1% Tween-20) at 4° C. overnight. Blots were cut and incubated with anti-MANDYS106 (1:50 dilution, Millipore clone 2C6), anti-HA (1:1000 dilution, Biolegend clone 16B12, or anti-GAPDH (1:5000 dilution, Cell Signaling clone 14C10) in 5% milk-TBST at room temperature for 1 hour. Blots were then washed in TBS-T and incubated with goat anti-mouse-conjugated horseradish peroxidase (1:2500 dilution, Sigma) or goat anti-rabbit-conjugated horseradish peroxidase (1:2500 dilution, Sigma) in 5% milk-TBS-T at room temperature for 1 hour. Blots were washed in TBST then visualized using Western-C ECL substrate (Bio-Rad) on a ChemiDoc XRS+ System (Bio-Rad).

Histological analysis. Muscles were dissected and embedded in OCT or flash-frozen using liquid nitrogen-cooled isopentane. Subsequently, 10 μm sections were cut onto pretreated histological slides using a cryostat (Leica). Slides were washed in PBS and blocked in PBS supplemented with 5% BSA, and 0.1% Triton X-100. Slides were stained with mouse anti-MANDYS106 (1:200 dilution, Millipore clone 2C6) and rabbit anti-Laminin (1:300 dilution, Sigma L9393) in blocking buffer at room temperature for 1 hour. Slides were washed 3× with PBS for 5 minutes and goat anti-mouse IgG2a, Alexa Fluor 594 (1:500 dilution, ThermoFisher A-21135) or goat anti-rabbit IgG (H+L), Alexa Fluor 488 (1:500 dilution, ThermoFisher A-11034) was applied with DAPI (1:1000 dilution) at room temperature for 1 hour. Slides were washed and mounted with ProLong Gold Antifade Mountant (Invitrogen) and imaged with a Zeiss AxioObserve 7 microscope. Total fibers (anti-Laminin) were counted using the analyze particles function on ImageJ (Schindelin, J., et al. Nat. Methods 2012, 9, 676-682) and human dystrophin-positive fibers (anti-MANDYS106) were manually counted from a series of 5 randomized images for each sample. Percent dystrophin-positive fibers were calculated as dystrophin-positive fibers divided by the total fibers for each image. P-values were calculated by nested global one-way ANOVA with Tukey post hoc multiple comparisons tests.

Off-target analysis. CIRCLE-seq libraries (Tsai, S. Q., et al. Nat. Methods 2017, 14, 607-614) were generated as previously described (Kocak, D. D., et al. Nat. Biotechnol 2019, 37, 657-666). Approximately 50-100 μg of HEK293T gDNA was used to generate circles for each reaction. Using a Diagenode Bioruptor XL sonicator at 4° C., gDNA was sonicated to an average size of approximately 50 bp, with a visible range of 200-1000 bp, as determined by agarose gel electrophoresis. The enzymatic procedure to generate circles was carried out as previously described (Tsai, S. Q., et al. Nat. Methods 2017, 14, 607-614). For the in vitro digest of the circles, gRNAs were synthesized from IDT and SaCas9 was purchased from Applied Biological Materials. Library production was carried out as previously described for CHANGE-seq (Lazzarotto, C. R., et al. Nat. Biotechnol. 2020, 38, 1317-1327). Libraries were quantified by the qPCR-based KAPA Library Quantification Kit (KAPA Biosystems), pooled, and sequenced with 150-bp paired-end reads on an Illumina NextSeq instrument. Read counts were obtained using previously described methods and software for CHANGE-seq (Lazzarotto, C. R., et al. Nat. Biotechnol. 2020, 38, 1317-1327). The following parameters were used for running the analysis pipeline: read threshold of 4, window size of 3, mapq threshold of 50, start threshold of 1, gap threshold of 3, mismatch threshold of 6, and PAM of NNGRRN (SEQ ID NO: 8; Ran, F. A., et al. Nature 2015, 520, 186-191).

Creatine kinase assay. Serum creatine kinase was measured using a Liquid Creatine Kinase Reagent set (Pointe Scientific) following the manufacturer's instructions. In brief, 5 μL of serum was diluted in 45 μL sterile PBS and incubated with reagent for 2 min at 37° C. followed by absorbance measurements taken every minute three readings using a nanodrop spectrophotometer set for 340 nm readings. Calculations for total creatine kinase in U/L were made according to the manufacturer's instructions.

Statistical analysis. All data was analyzed with four to six biological replicates and presented as mean±SEM. All p-values were calculated by global one-way ANOVA with Tukey post hoc tests (α=0.05).

### Example 2

### Correction Strategy for Humanized Mouse Model of DMD

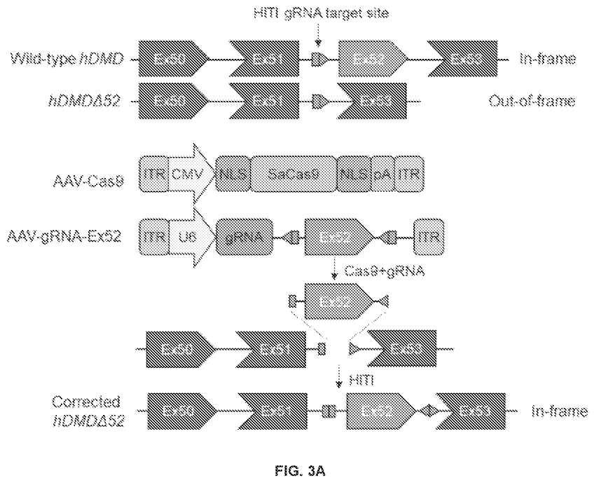

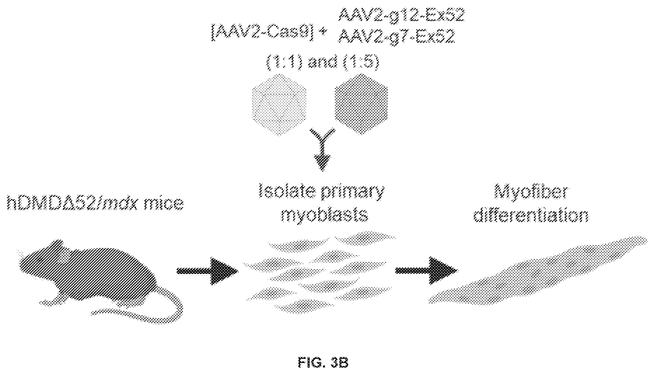

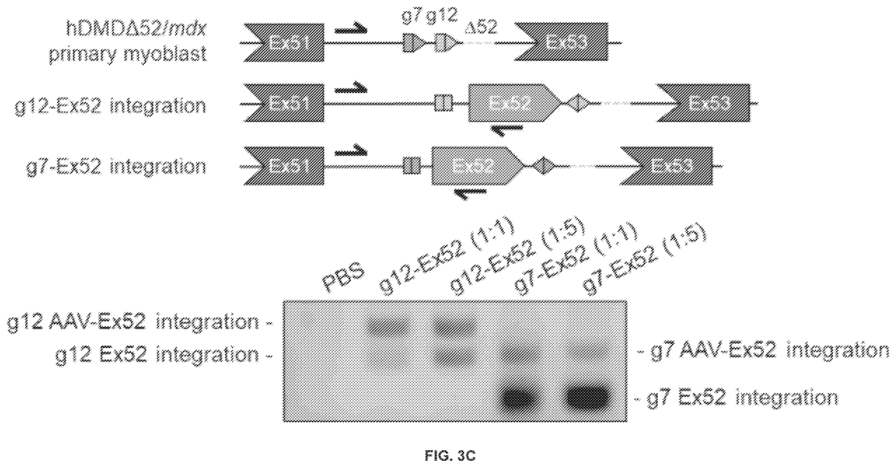

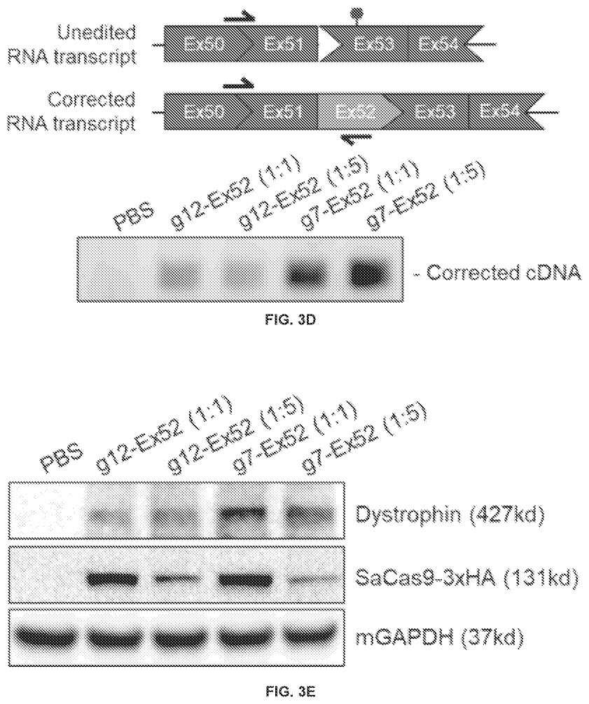

The hDMD/mdx mouse lacks mouse dystrophin due to the hallmark mdx mutation but produces human dystrophin from the full-length human DMD (hDMD) gene on mouse chromosome 5. These mice can be used to generate humanized DMD mouse models by removing hDMD exons known to be missing in patient populations, and thus eliminating all dystrophin expression. Importantly, these humanized models can be used to test therapeutic strategies because human dystrophin restoration can functionally compensate for the lack of mouse dystrophin. To study various gene editing therapeutic strategies, a hDMDΔ52/mdx mouse model was generated by delivering Streptococcus pyogenes Cas9 (SpCas9) and gRNAs to hDMD/mdx zygotes for targeted exon 52 deletion from the hDMD gene. Deletion of exon 52 results in an out-of-frame mutation (FIG. 3A) that creates a premature stop codon and subsequent loss of dystrophin expression. To restore full-length dystrophin expression, a HITI-based approach was developed to insert exon 52 at its corresponding position in the hDMD gene in this humanized hDMDΔ52/mdx mouse model. This dual AAV vector approach includes one AAV vector that encodes a Staphyloccocus aureus Cas9 (SaCas9) (Ran, F. A., et al. Nature 2015, 520, 186-191) expression cassette and a second AAV vector that encodes a gRNA expression cassette with the exon 52 donor sequence (Ex52) flanked by the same gRNA target site found in intron 51 of the hDMD gene. Following co-delivery of both AAV vectors, Cas9 and the gRNA was expressed and created a DSB at the genomic target site, as well as liberated the Ex52 donor sequence from the AAV vector so that following NHEJ-based repair, the exon 52 sequence integrated into the target site and restore a full-length dystrophin gene. The gRNA target sites were in opposite orientation in the genomic DNA and AAV vector so that correct donor integration disrupted the gRNA target sequence and prohibited further Cas9-based editing (FIG. 3A).

### Example 3

### Screening and Validation of HITI-Mediated Integration

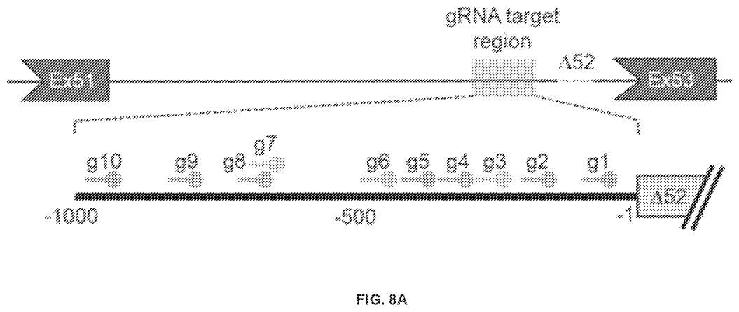

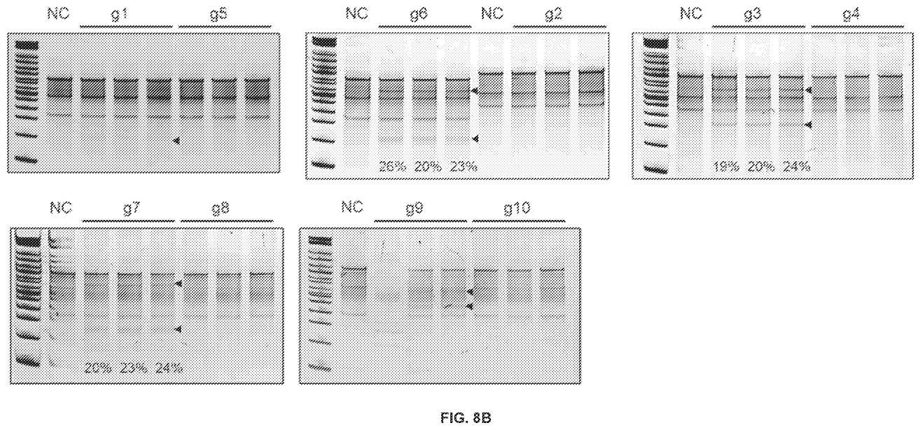

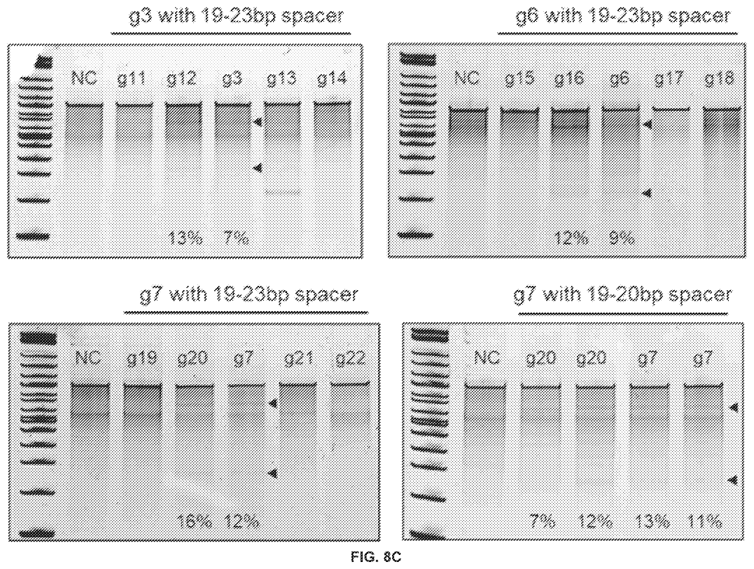

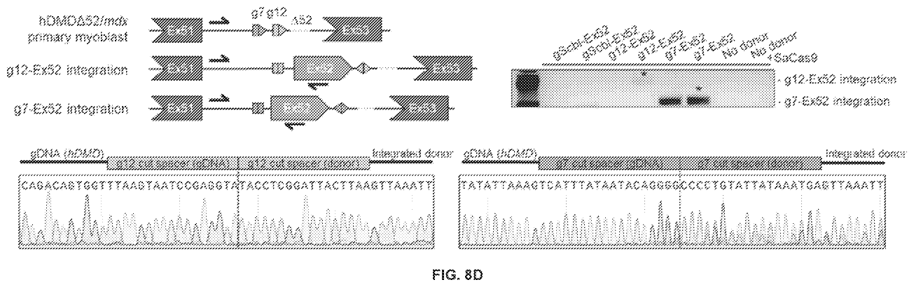

The specificity of DNA cleavage by the CRISPR-Cas9 system is critical to ensuring the safety and efficacy of this approach. To minimize potential off-target effects, bioinformatic analysis was used to design gRNAs with limited predicted off-target sites in murine and human genomes. A panel of SaCas9 gRNAs targeting intron 51 (FIG. 8A, TABLE 1) was screened to identify targets with high specificity and activity, initially using the Surveyor assay following plasmid transfection of HEK293T cells (FIG. 8B). SaCas9 activity can vary across a range of spacer lengths, therefore 19-23 nt spacers of the top gRNAs were generated and individually screened for activity by Surveyor assay, following plasmid electroporation into DMD patient myoblasts (FIG. 8C). The individual gRNA sequences with the highest activity levels and fewest predicted off-target sites (g12 and g7), along with a scrambled non-target control gRNA (gScbI), were cloned into AAV vector plasmids for gRNA expression and the corresponding spacer and PAM target sequences were included flanking the donor sequence.

To validate targeted integration of the Ex52 donor sequence, primary myoblasts were isolated from hDMDΔ52/mdx skeletal muscle (FIG. 3B). Following electroporation of AAV plasmids, targeted Ex52 integration in genomic DNA (gDNA) was confirmed by Sanger sequencing of the PCR amplified genome-donor junction for g12-Ex52 and g7-Ex52 treated hDMDΔ52/mdx primary myoblasts, but not gScbI-treated cells (FIG. 8D). To validate AAV-mediated targeted integration and subsequent correction of dystrophin transcripts and protein restoration, primary myoblasts were transduced with AAV2 and then cultured the cells in differentiation conditions to upregulate dystrophin expression (FIG. 3B). In addition to delivering both AAV2 constructs at equal doses (1:1), delivery of 5× more AAV-donor than AAV-Cas9 (1:5) was also tested. Total volume of AAV preps remained consistent for 1:1 and 1:5 treatment groups, resulting in delivery of more AAV-Cas9 viral genomes for the 1:1 treatment group. Using both delivery ratios, targeted Ex52 integration was confirmed in gDNA by PCR amplification of the genome-donor junction (FIG. 3C). The presence of a larger amplicon was detected, which was confirmed to be intact AAV-donor integration by Sanger sequencing (data not shown). Additionally, the presence of Ex52 was observed in dystrophin cDNA following PCR amplification (FIG. 3D) and resulted in dystrophin protein restoration (FIG. 3E). The higher Cas9 expression observed in 1:1 treated cells correlated with the higher AAV-Cas9 viral dose. These results confirm activity of the AAV-Cas9-based strategy for targeted Ex52 integration and full-length dystrophin protein restoration.

### Example 4

### AAV-Cas9 Exon 52 Integration Restores Full-Length Dystrophin In Vivo

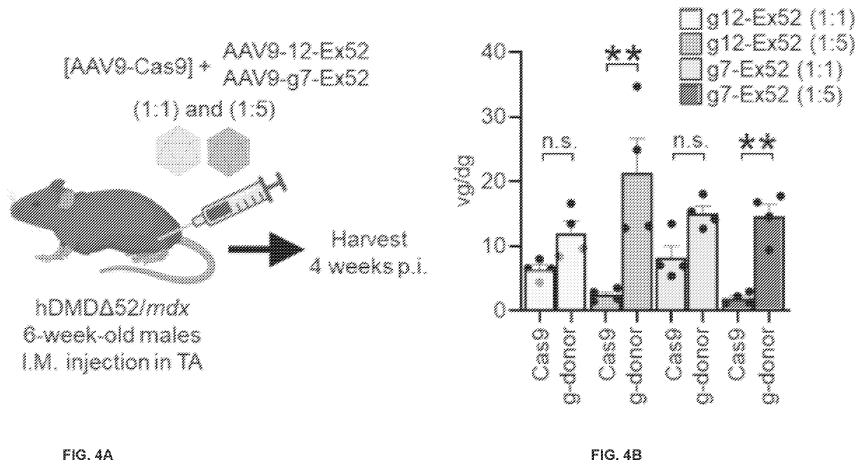

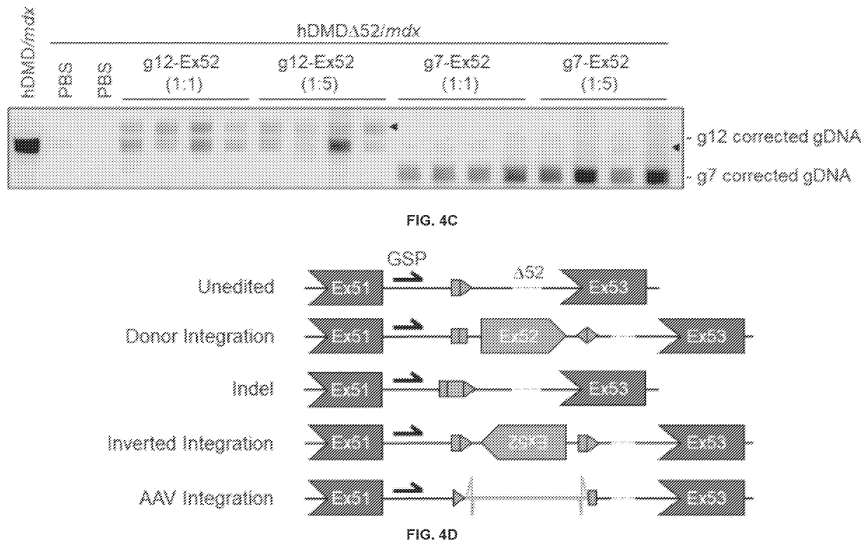

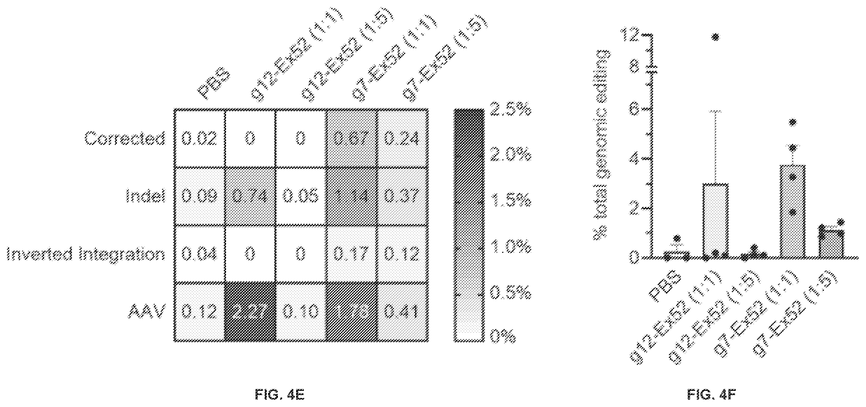

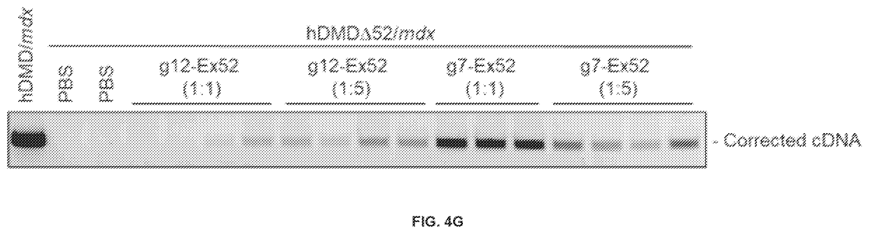

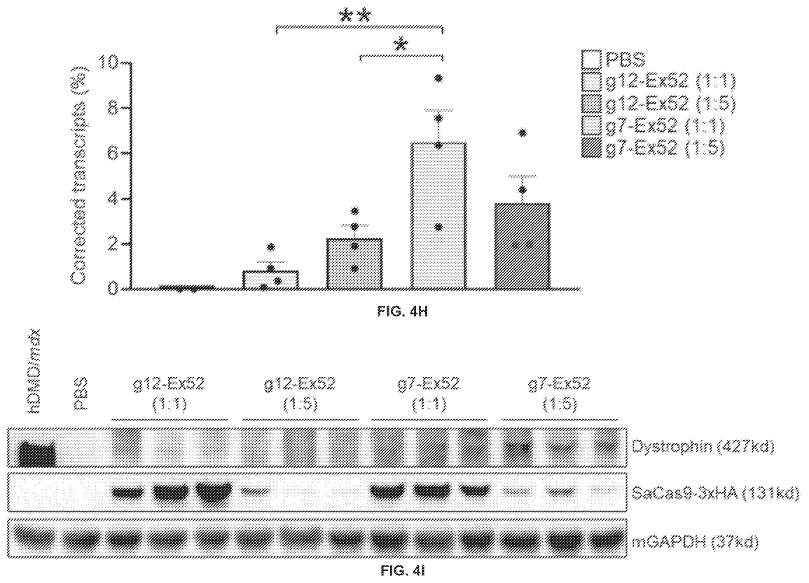

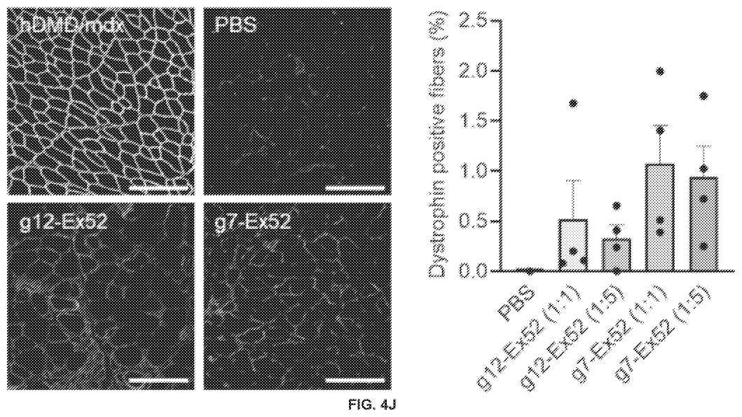

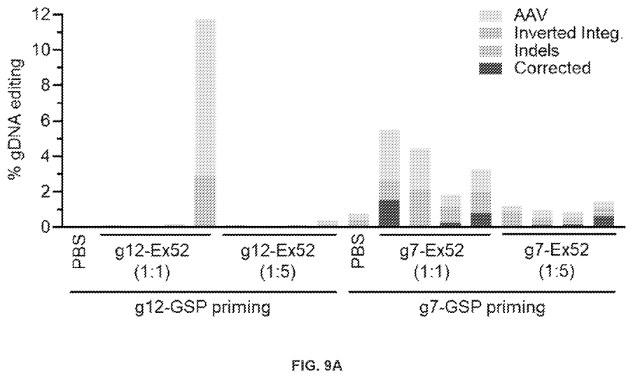

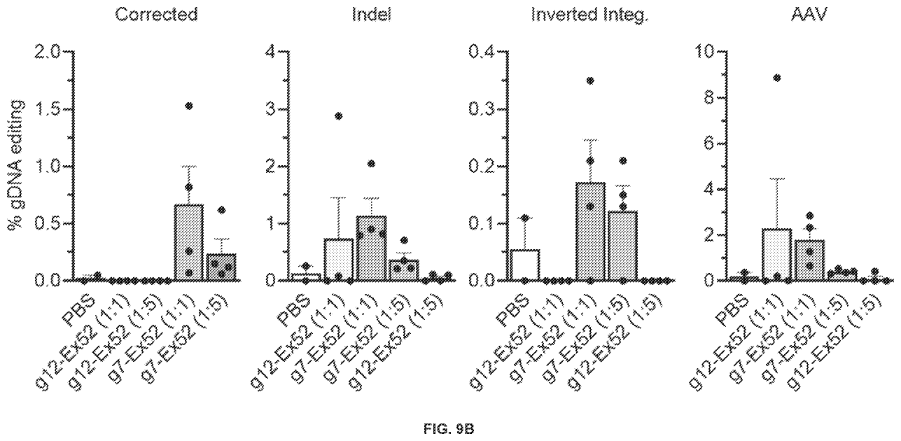

AAV9 was used for delivery of the CRISPR-Cas system to hDMDΔ52/mdx mouse skeletal and cardiac muscle. Following co-injection of the two AAV vectors into the tibialis anterior (TA) muscle of adult hDMDΔ52/mdx male mice (FIG. 4A), local AAV vector delivery at comparable levels was confirmed for both g12 and g7 vectors by digital droplet PCR (ddPCR) of DNA vector genomes (FIG. 4B). Targeted Ex52 integration was confirmed in gDNA from TA tissue by PCR amplification of the genome-donor junction using both AAV delivery ratios and intact AAV-donor integration (FIG. 4C). To quantify editing activity and comprehensively map possible genome editing outcomes with an unbiased approach, Tn5-transposon-based library preparation methods (Nelson, C. E., et al. Nat. Med. 2019; Giannoukos, G., et al. BMC Genomics 2018, 19, 212) were adapted and included unique molecular identifiers (UMI) to remove PCR duplicates for increased accuracy of quantifying rare events. The Tn5-based method eliminated PCR biases associated with target specificity and amplicon length by using a single genome-specific primer (GSP) adjacent to a gRNA cut site in combination with a transposon-specific primer for the Tn5-integrated DNA tag. In addition to quantifying donor integrations in the correct orientation, genome editing events were measured that included indels, donor inversion integrations, and AAV-ITR integrations (FIG. 4D). Higher correction and total genomic editing events were measured in g7 treated mice (FIG. 4E, FIG. 4F, FIG. 9A, and FIG. 9B), however indel and AAV integration edits were also observed in g12 treated mice (FIG. 4E, FIG. 9A, and FIG. 9B). Although corrected genomic reads were not detected in g12 treated mice, the presence of exon 52 was observed in corrected dystrophin cDNA following PCR amplification (FIG. 4G) and quantified by ddPCR (FIG. 4H) for all treatment groups. Full-length dystrophin restoration was confirmed by Western blot of whole TA tissue lysates (FIG. 4I) and dystrophin positive fibers were quantified by immunofluorescence (IF) (FIG. 4J). These results confirmed in vivo activity of the AAV-Cas9-based strategy for targeted Ex52 integration and full-length dystrophin protein restoration following local injection in hDMDΔ52/mdx mouse skeletal muscle.

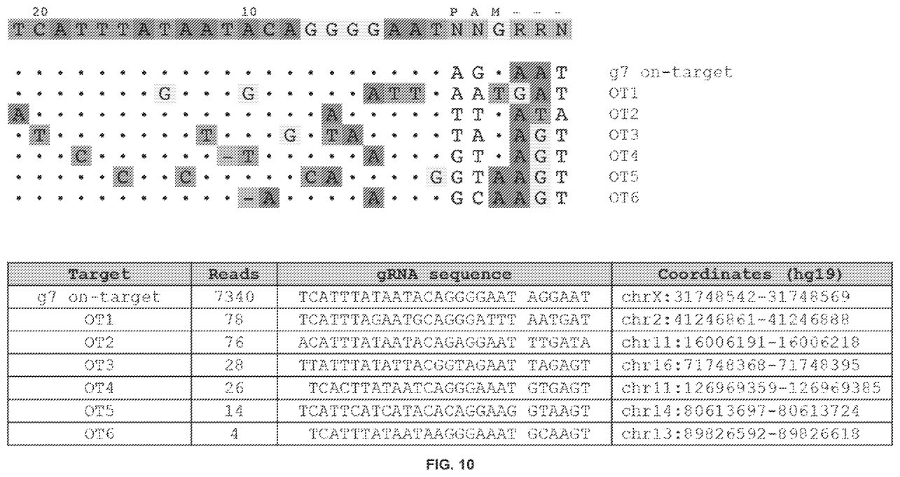

Assessing CRISPR-Cas targeting specificity may be important for pre-clinical development. Collectively, greater genome editing activity and subsequent dystrophin restoration was measured for g7-treated mice (FIG. 3A-3F), thus the specificity analyses were focused on g7. To empirically determine the top g7 off-target sites in the human genome with an unbiased genome-wide assay, high-throughput genome-wide editing quantification was performed (Tsai, S. Q., et al. Nat. Methods 2017, 14, 607-614; Lazzarotto, C. R., et al. Nat. Biotechnol. 2020, 38, 1317-1327) (FIG. 10). These analyses identified 6 potential off-target sites with editing activity≤1.07% of on-target gDNA editing, confirming high specificity of this g7 gRNA. For the remainder of the work, g7-targeted full-length dystrophin restoration strategies were focused on.

### Example 5

### In Vitro Validation of AAV-Cas9 Superexon Strategy for Full-Length Dystrophin Restoration

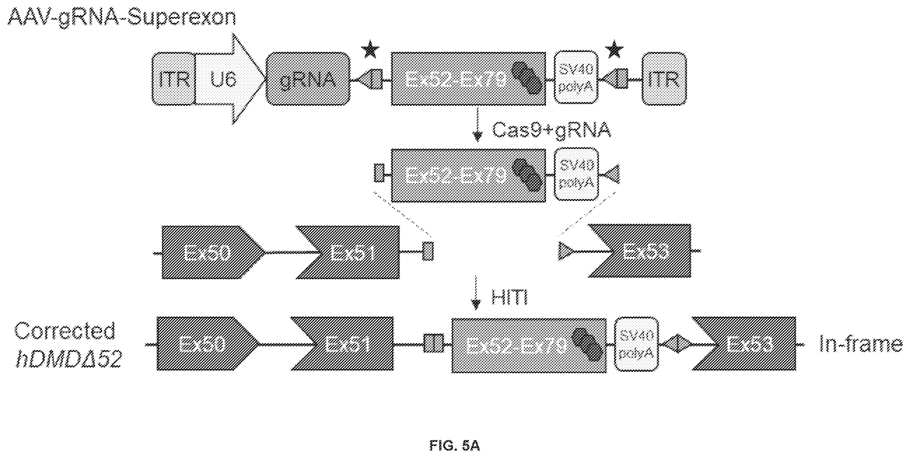

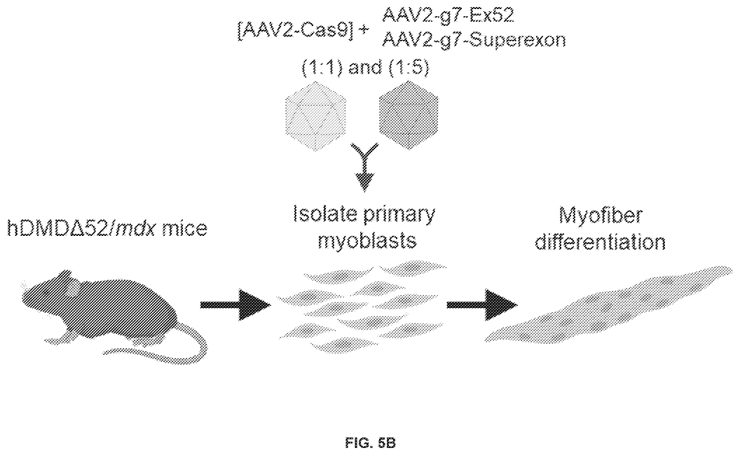

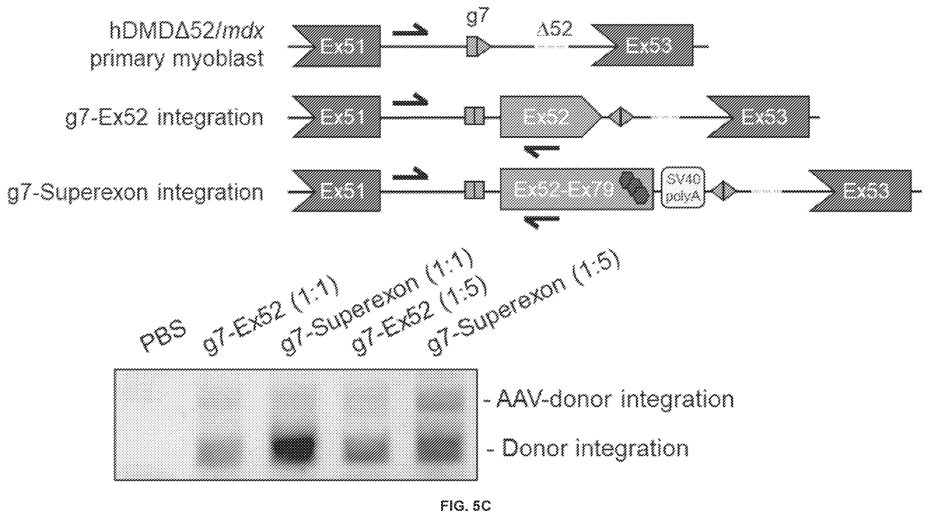

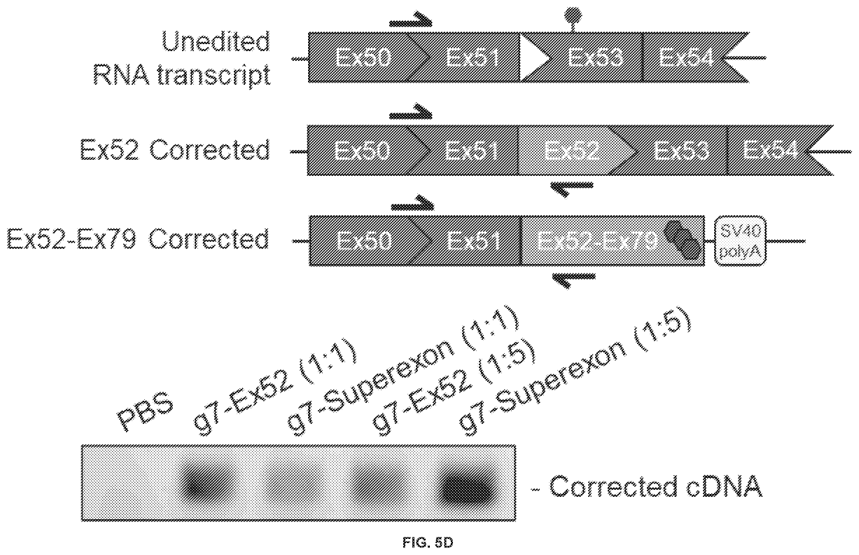

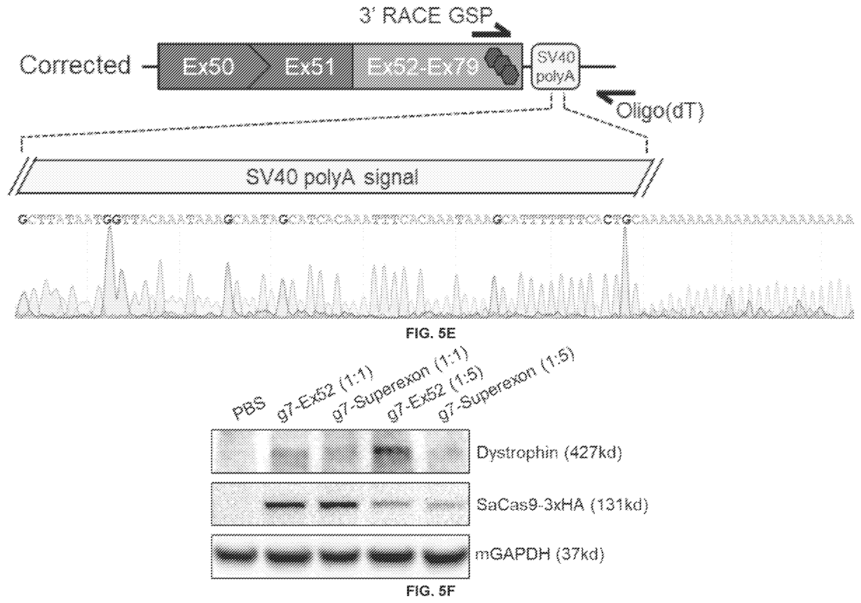

The g7-Ex52 integration approach can correct full-length dystrophin for Δ52 DMD patients and restore the proper reading frame to produce a truncated dystrophin protein for Δ52-58, Δ52-61, and Δ52-76 patient mutations. To expand the full-length dystrophin correction strategy to treat any genetic mutation downstream of exon 51, an AAV-superexon donor vector was engineered. This superexon encoded the complete dystrophin cDNA coding sequence downstream of exon 51, including exons 52 through 79. Additionally, the stop codon was replaced with a 3× stop to ensure translation termination in all reading frames, included the SV40 polyA sequence, and flanked the donor cassette with the previously validated g7 target sites (FIG. 5A). Targeted integration of this g7-superexon construct could correct full-length dystrophin in >20% of all DMD patients.

To validate superexon integration and subsequent correction of dystrophin transcripts and protein restoration, primary myoblasts were transduced with AAV2 at 1:1 and 1:5 vector ratios, then the cells were cultured in differentiation conditions to upregulate dystrophin expression (FIG. 5B). Targeted integration was confirmed in gDNA by PCR amplification of the genome-donor junction for all treated samples, in addition to detection of intact AAV-donor integration (FIG. 5C). The presence of exon 52 from both donors was observed at comparable levels in dystrophin cDNA following PCR amplification (FIG. 5D). For wild-type dystrophin transcripts, almost 2.7 kb of untranslated region (3′ UTR) was included in exon 79 following the stop codon. In the superexon donor, this sequence was replaced with a shortened polyA signal due to the packaging size restrictions of AAV (˜4.7kb). To characterize this engineered 3′ UTR, 3′ RACE was performed using cDNA of AAV-transduced cells and superexon-corrected dystrophin transcripts were amplified using a GSP that recognizes the engineered 3× stop (FIG. 5E). Following Sanger sequencing, addition of a polyA tail was observed within the SV40 polyA signal sequence. Next, it was confirmed that superexon-corrected dystrophin transcripts resulted in dystrophin protein restoration (FIG. 5F). These results confirmed activity of the targeted AAV-Cas9-based Ex52-79 superexon integration strategy for full-length dystrophin protein restoration.

### Example 6

### AAV-Cas9 Superexon Strategy Restores Full-Length Dystrophin in Skeletal Muscle and Cardiac Muscle

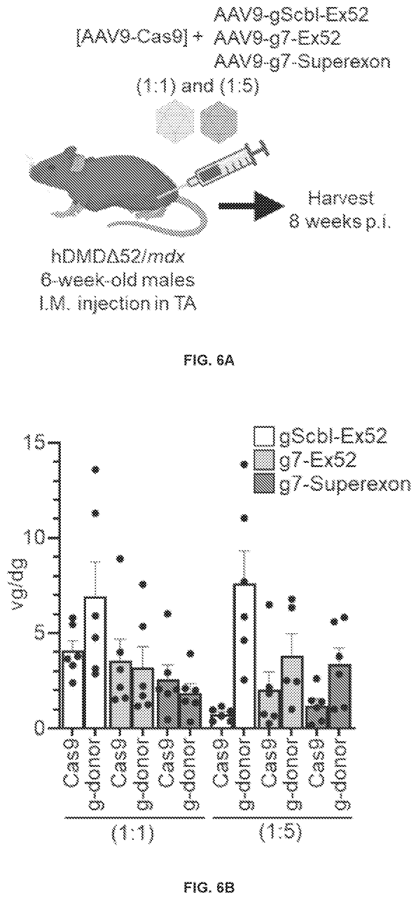

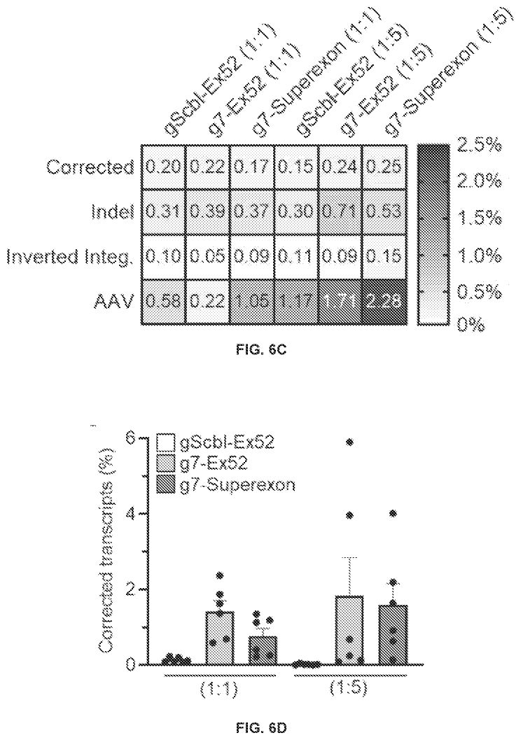

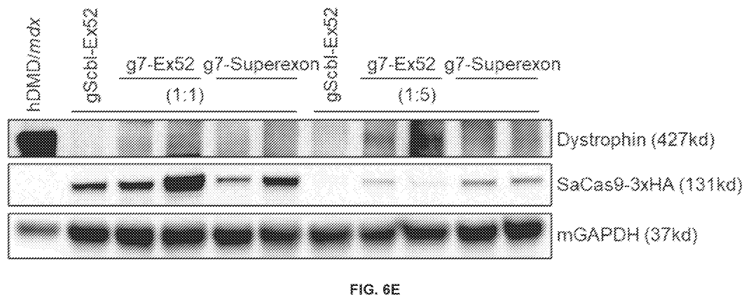

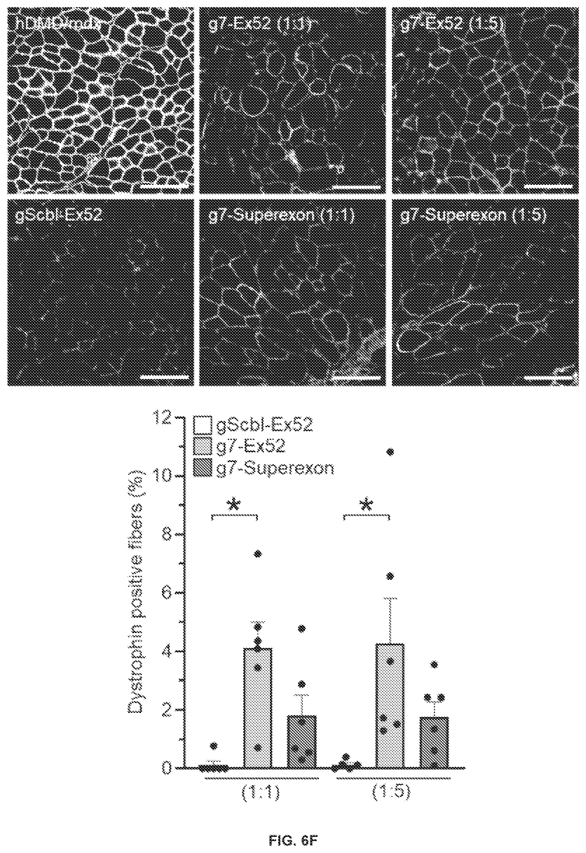

To test the Superexon strategy in vivo, the AAV9 constructs were co-injected at a ratio of 1:1 and 1:5 into the TA muscle of adult hDMDΔ52/mdx male mice (FIG. 6A). A scrambled non-target gRNA donor (gScbI-Ex52) was included as an additional control. At 8 weeks post-injection, equivalent AAV vector genome levels between treatment groups were measured by ddPCR (FIG. 6B). Targeted editing activity was quantified using Tn5-based library preparation and analysis methods with the highest editing levels in the 1:5 treated mice. The lower g7-Ex52 editing levels observed in this donor comparative study (FIG. 6C and FIG. 11A-11B), in contrast to the previous gRNA comparative study (FIG. 4E and FIG. 9A-9B), was likely due to lower AAV transduction in the TA as demonstrated by differences in vector genome quantification (FIG. 4B and FIG. 6B). Although corrected gDNA levels were not detected above background by Tn5 analysis, an increase in the percent of corrected transcripts was observed for all treatment groups (FIG. 6D). Additionally, full-length dystrophin expression was observed by Western blot (FIG. 6E) and IF (FIG. 6F) and quantification of dystrophin positive fibers resulted in a significant increase for g7-Ex52 treated mice compared to the scrambled non-targeted donor control. These results confirmed in vivo activity of the AAV-Cas9-based strategy for targeted superexon integration and full-length dystrophin protein restoration following local injection in hDMDΔ52/mdx mouse skeletal muscle.

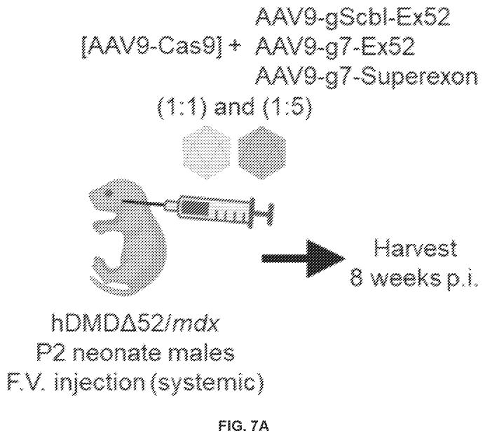

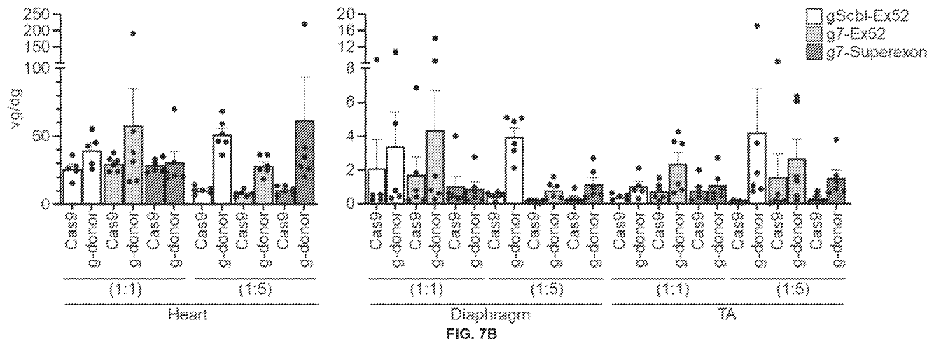

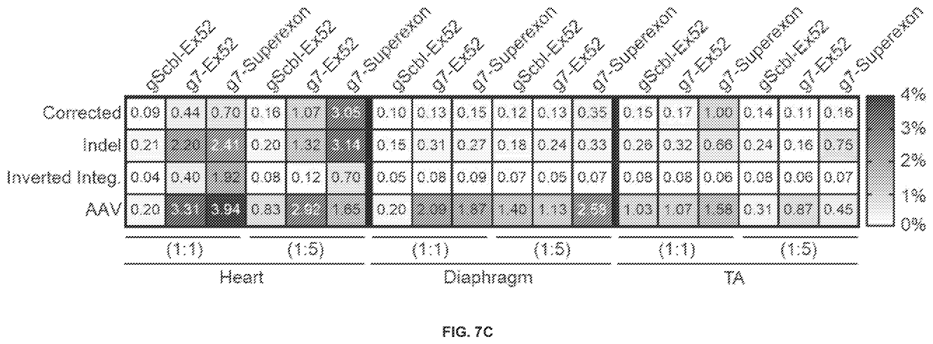

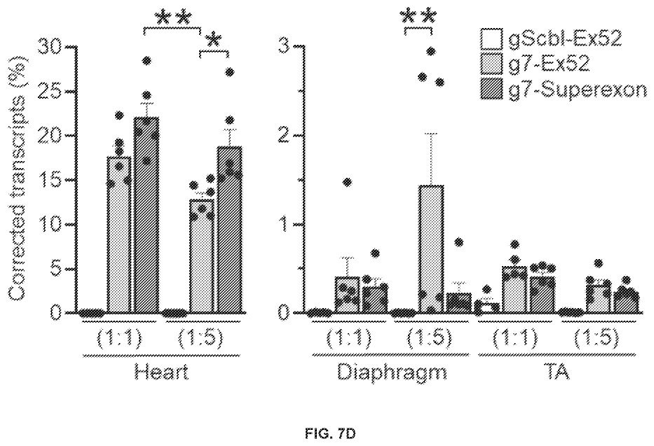

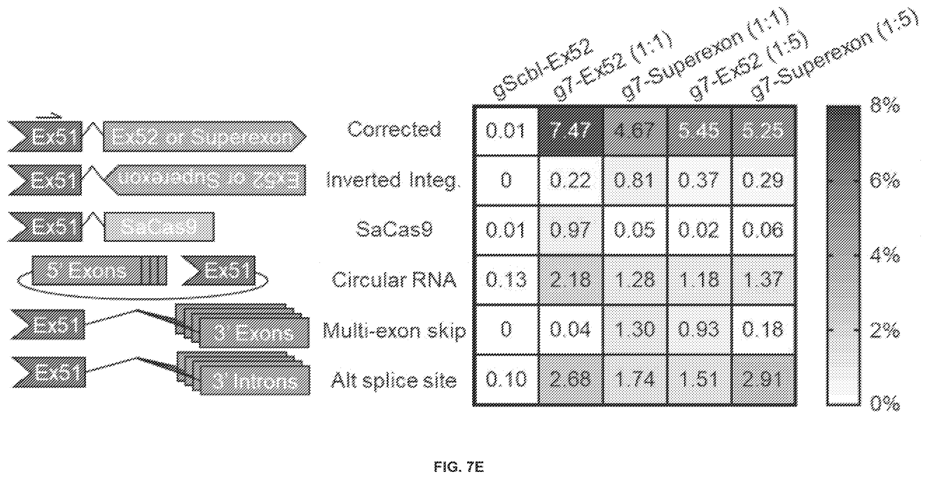

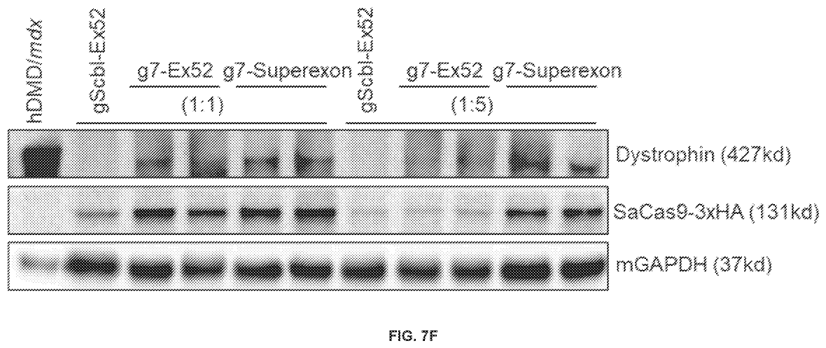

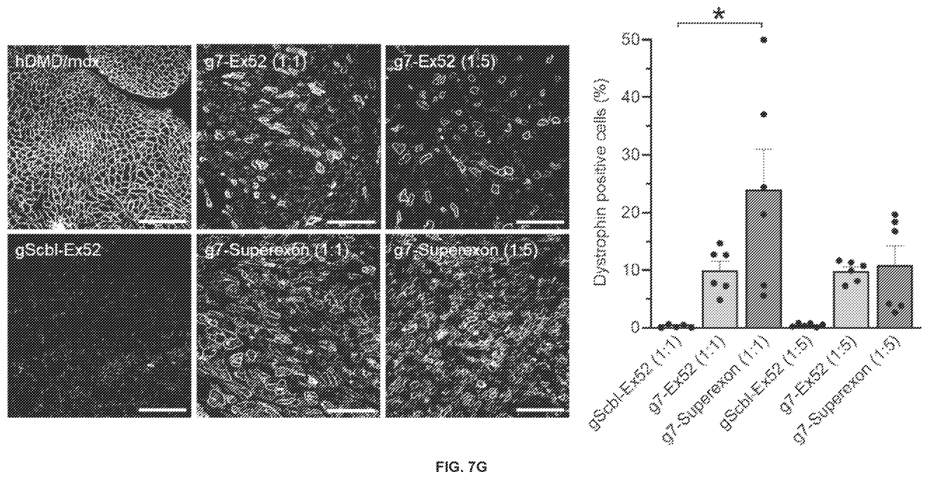

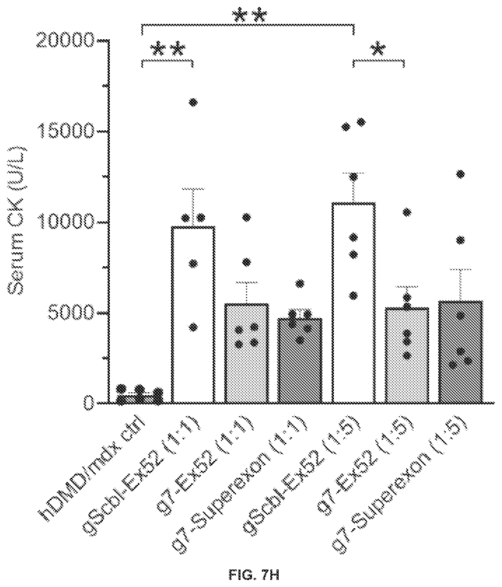

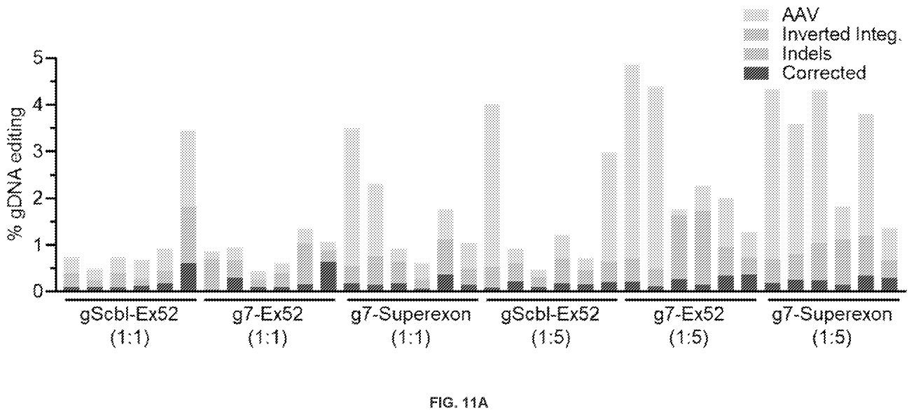

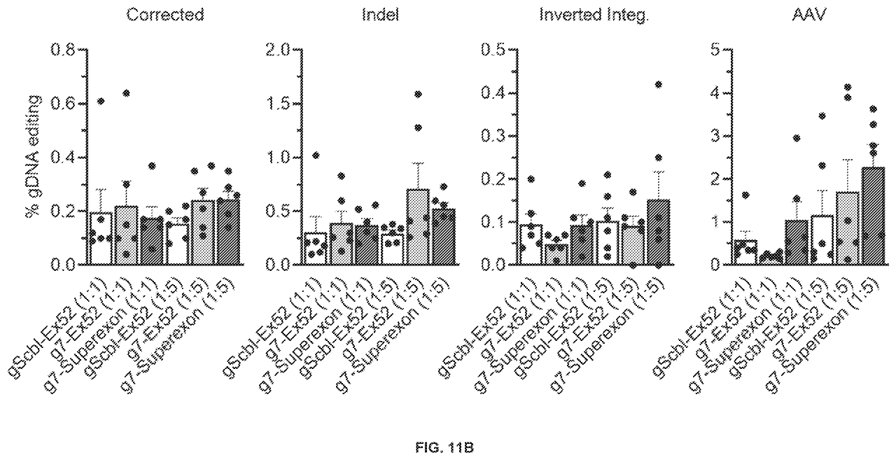

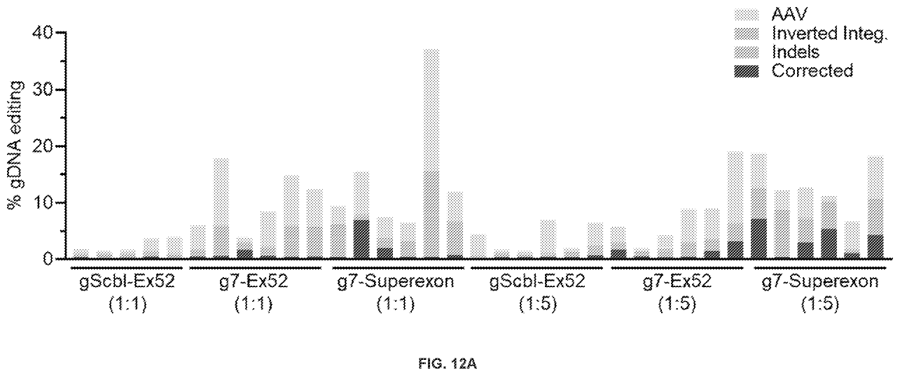

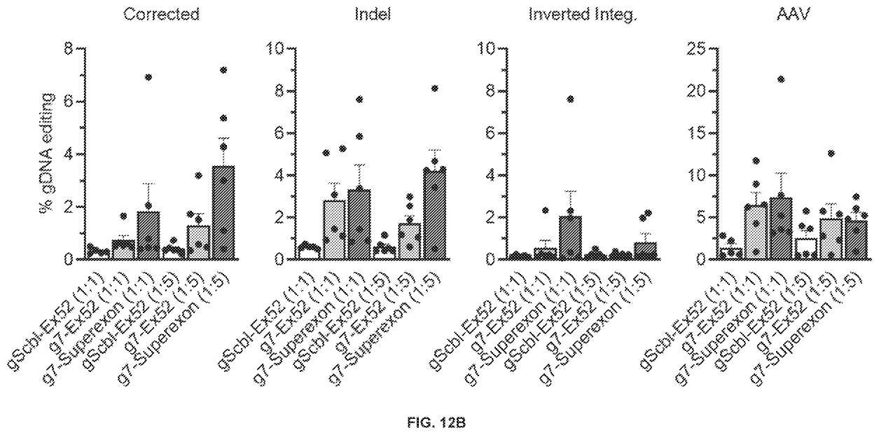

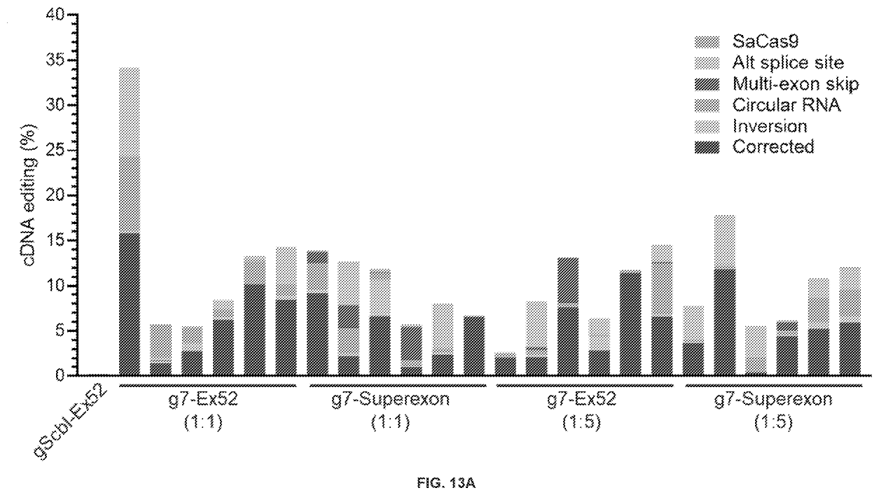

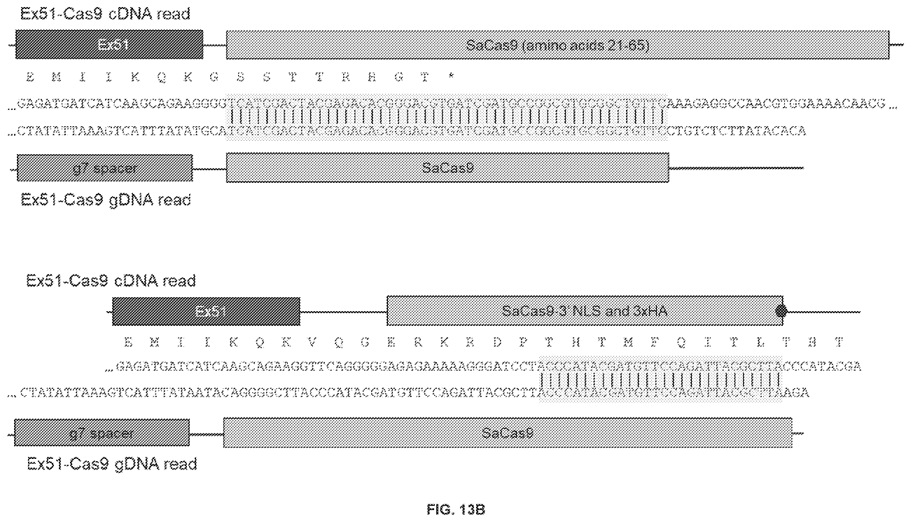

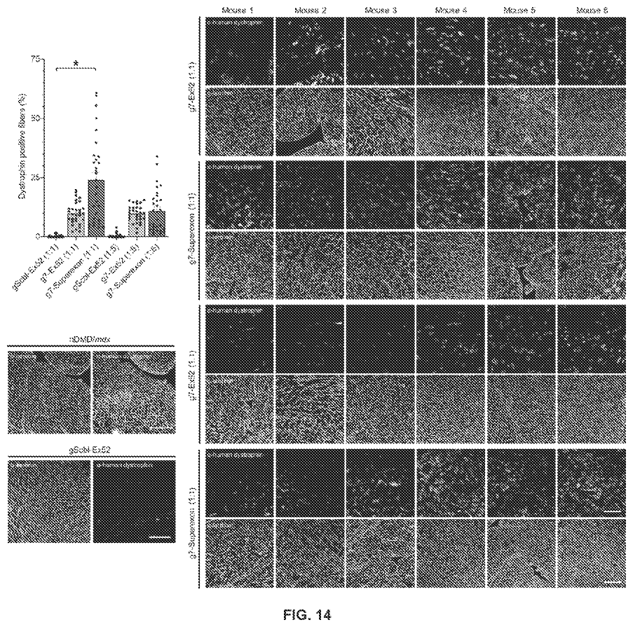

Next, the corrective therapeutic potential of these integration strategies was evaluated following systemic delivery. For transduction of cardiac and skeletal muscle, the AAV9 constructs were co-delivered at a ratio of 1:1 and 1:5 by facial vein injection of P2 neonate hDMDΔ52/mdx male mice (FIG. 7A). At 8 weeks post-injection, vector genome quantification by ddPCR revealed higher transduction levels in cardiac tissue than skeletal (diaphragm and TA) tissues (FIG. 7B), suggesting the potential for higher editing activity in hearts of treated mice. Indeed, Tn5-based quantification revealed higher editing for all quantified outcomes in the heart gDNA compared to diaphragm and TA, with the highest on-target correction in hearts of g7-Superexon treatment groups (FIG. 7C and FIG. 12A-12B). Higher levels of corrected dystrophin transcripts were observed in hearts of g7-Superexon treatment groups with mice achieving >25% corrected transcripts (FIG. 7D). The ddPCR-based transcript quantification was limited to detection of unedited (Ex51-Ex53 junction) and corrected (Ex51-Ex52 junction) cDNA molecules. For additional heart transcript characterization, putative aberrant splicing events were measured that included inversion donor integrations, splicing with the SaCas9 coding sequence, circular RNA formation (exons 1-51), multi-exon skipping (exons 53-79), and alternative splicing with downstream intronic sequences (introns 51-53) (FIG. 7E). Higher levels of editing were measured by ddPCR than Tn5-based deep sequencing strategies. Lower deep sequencing editing percentages may be attributed by larger denominators generated from measuring unintended gene editing outcomes. For cDNA analysis, the high-throughput deep sequencing characterization revealed considerable aberrant splicing with the SaCas9 coding sequence in two treated mice (FIG. 13A). Upon further investigation, genomic integration of aligned sequences in corresponding genomic mouse samples was confirmed (FIG. 13B). Transcript isoforms that contain partial AAV genomes, including partial SaCas9 coding sequences, have an unknown biological effect and could be investigated in future studies. In heart tissue, full-length dystrophin restoration was confirmed by Western blot (FIG. 7F) and dystrophin-positive cells were detected in all treated mice (FIG. 7G and FIG. 14). A significant increase in dystrophin-positive cells was observed for g7-superexon (1:1) treated mice compared to the scrambled non-targeted gRNA donor control, with almost 50% of dystrophin-positive cells observed for one mouse. Serum creatine kinase levels, a marker of muscle degeneration, were significantly higher for control hDMDΔ52/mdx mice compared to hDMD/mdx mice, suggestive of a diseased DMD phenotype (FIG. 7H). Additionally, serum creatine kinase levels were reduced in hDMDΔ52/mdx mice after all systemic treatments, demonstrating protection from muscle damage by the restored full-length dystrophin protein.

### Example 7

### Discussion

DMD gene therapy strategies have been explored for nearly 30 years, however strategies to correct full-length dystrophin are lacking. In this study, use of targeted HITI-mediated transgene insertion was demonstrated for full-length human dystrophin correction and restoration in hDMDΔ52/mdx mice. A dual AAV delivery system was used for generating a Cas9-targeted genomic DSB and delivering donor sequences for NHEJ-mediated integration at the cut site. Here, the therapeutic potential of NHEJ-mediated integration approaches following both local injection and systemic delivery in skeletal and cardiac tissues was demonstrated. Additionally, high-throughput unbiased sequencing was performed to characterize and quantify genomic and transcriptional editing events.

Although downstream consequences of HITI-mediated correction resulted in dystrophin protein restoration, the deep sequencing results demonstrate low genomic correction efficiency. The observation of high dystrophin protein restoration resulting from low genomic editing efficiency is consistent with alternative DMD gene editing approaches. While low levels of dystrophin expression, even less than 4% of normal, can result in potential therapeutic benefit, improving HITI efficiency will aid translation of knock-in gene therapy strategies to clinical applications. The gene editing strategy detailed herein may be similar to other gene transfer strategies in its need for robust delivery to targeted tissues and cells. Methods to improve AAV-mediated tissue-specific transduction and expression, such as AAV capsid evolution and promoter engineering, may improve gene editing activity and therapeutic potential. Additionally, targeted integration in dividing and non-dividing cells may be increased by identification of NHEJ regulators leading to the development of small molecule targets for enhancing HITI-mediated activity. Alternatively, other targeted gene knock-in methods can be explored including microhomology-mediated end-joining (MMEJ), Precise Integration into Target Chromosome (PiTCh), homology-mediated end joining (HMEJ), and intercellular linearized Single homology Arm donor mediated intron-Targeting Integration (SATI). With continued progress in editing efficiency, HITI-mediated transgene knock-in holds great promise for future development of corrective gene therapy strategies.

Pre-clinical gene editing studies may benefit from use of humanized mouse models because they permit testing of therapeutic approaches specifically designed to treat human patients. To apply HITI-based gene therapy strategies to a DMD disease model that recapitulates mutations found in patients, hDMDΔ52/mdx mice were utilized, which contain a gene deletion in the DMD patient mutational hotspot of exons 45-55. Full-length protein restoration was demonstrated following targeted integration of the missing exon 52 coding sequence. To expand this corrective gene therapy approach to a larger patient population (>20%), a superexon encoding the complete human dystrophin cDNA coding sequence downstream of exon 51 was engineered that can correct all patient mutations located after exon 51, and demonstrated full-length protein restoration using this approach. This work is the first demonstration of a targeted gene editing approach to permanently correct full-length dystrophin. This approach will be extended to all patients with mutations within and downstream of the exon 45-55 hotspot (>50% of all patients), for example, with a dual AAV-based system with one AAV that encodes SaCas9 and a gRNA targeting intron 44, and a second donor AAV vector that contains the human dystrophin cDNA coding sequence downstream of exon 44 (exons 45-79). Sequences for gRNAs targeting intron 44 are shown in TABLE 3. Exons 45-79 of the human dystrophin gene may be encoded by a polynucleotide of SEQ ID NO: 154, and an example of a donor sequence for insertion of exons 45-79 is shown in SEQ ID NO: 155.

TABLE 3
Examples of gRNAs targeting intron 44 of human
dystrophin gene.

| DNA encoding gRNA | gRNA |
|---|---|
| GGGGCTCCACCCTCACGAGT | GGGGCUCCACCCUCACGAGU |
| (SEQ ID NO: 157) | (SEQ ID NO: 171) |
| GCACAAAAGTCAAATCGGAA | GCACAAAAGUCAAAUCGGAA |
| (SEQ ID NO: 158) | (SEQ ID NO: 172) |
| GATTTCAATATAAGATTCGG | GAUUUCAAUAUAAGAUUCGG |
| (SEQ ID NO: 159) | (SEQ ID NO: 173) |
| GTGAGGGCTCCACCCTCACGA | GUGAGGGCUCCACCCUCACGA |
| (SEQ ID NO: 160) | (SEQ ID NO: 174) |
| GAAGGATTGAGGGCTCCACCC | GAAGGAUUGAGGGCUCCACCC |
| (SEQ ID NO: 161) | (SEQ ID NO: 175) |
| GGCTCCACCCTCACGAGTGGG | GGCUCCACCCUCACGAGUGGG |
| (SEQ ID NO: 162) | (SEQ ID NO: 176) |
| GTGAGGGCTCCACCCTCACGA | GUGAGGGCUCCACCCUCACGA |
| (SEQ ID NO: 163) | (SEQ ID NO: 177) |
| GGGCTCCACCCTCACGAGT | GGGCUCCACCCUCACGAGU |
| (SEQ ID NO: 164) | (SEQ ID NO: 178) |
| CACAAAAGTCAAATCGGAA | CACAAAAGUCAAAUCGGAA |
| (SEQ ID NO: 165) | (SEQ ID NO: 179) |
| ATTTCAATATAAGATTCGG | AUUUCAAUAUAAGAUUCGG |
| (SEQ ID NO: 166) | (SEQ ID NO: 180) |
| TGAGGGCTCCACCCTCACGA | UGAGGGCUCCACCCUCACGA |
| (SEQ ID NO: 167) | (SEQ ID NO: 181) |
| AAGGATTGAGGGCTCCACCC | AAGGAUUGAGGGCUCCACCC |
| (SEQ ID NO: 168) | (SEQ ID NO: 182) |
| GCTCCACCCTCACGAGTGGG | GCUCCACCCUCACGAGUGGG |
| (SEQ ID NO: 169) | (SEQ ID NO: 183) |
| TGAGGGCTCCACCCTCACGA | UGAGGGCUCCACCCUCACGA |
| (SEQ ID NO: 170) | (SEQ ID NO: 184) |

The engineered superexon donor encodes a shortened polyA signal to ensure proper transcriptional signals during mRNA generation from corrected genomic edits. The 3′ RACE characterization confirmed the addition of a polyA tail in superexon-corrected transcripts (FIG. 4E). Future efforts aimed to engineer superexon donors with 3′ UTRs optimized for mRNA stability may result in enhanced therapeutic potential. HITI-mediated single exon and superexon gene editing approaches can also be applied to other genetic diseases including those with gene targets, like DMD, that may be too large to fully package in AAV delivery vectors or characterized by a wide-spectrum of patient mutations, including hemophilia, cystic fibrosis, and Neurofibromatosis type 1.

Previously, proof-of-principle HITI-mediated gene corrective strategies characterized editing outcomes by Surveyor analysis, in-out PCR amplification, ddPCR, and TOPO sequencing. In this study, Tn5-transposon-based library preparation methods and unbiased deep sequencing characterization were used for greater resolution of diverse HITI-mediated editing outcomes. Using these methods, genomic site-specific integration of intended donor corrections, inverted donor insertions, indels, AAV-ITR integrations, and AAV-Cas9 coding sequences were detected. Analysis of cDNA edits demonstrated on-target intended splicing and aberrant splicing including intended donor inclusion, inverted donor sequences, Cas9 coding sequences, circular RNAs, multi-exon skips, and alternative splice sites in downstream introns. These results are all consistent with previous observations of unintended editing outcomes using AAV-CRISPR.

Gene editing technologies have garnered incredible enthusiasm for the potential to correct genetic mutations to restore healthy, wild-type gene sequences. However, the majority of gene editing strategies being advanced to clinical trials today involve gene disruption, activation of compensatory factors, introduction of therapeutic genes to non-native “safe-harbor” loci, or the creation of truncated, partially functional gene sequences. The compositions and methods detailed herein represent an important step towards realizing the full potential of genome editing to treat the fundamental cause of genetic disease.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A CRISPR/Cas-based genome editing system comprising one or more vectors encoding a composition, the composition comprising: (a) a guide RNA (gRNA) targeting a fragment of a mutant dystrophin gene, wherein the gRNA hybridizes to a target sequence within intron 51 or intron 44 of the mutant dystrophin gene; (b) a Cas protein or a fusion protein comprising the Cas protein; and (c) a donor sequence comprising a fragment of a wild-type dystrophin gene, wherein the donor sequence comprises exon 52 of the wild-type dystrophin gene.

Clause 2. A CRISPR/Cas-based genome editing system comprising: (a) a guide RNA (gRNA) targeting a fragment of a mutant dystrophin gene, wherein the gRNA hybridizes to a target sequence within intron 51 or intron 44 of the mutant dystrophin gene; (b) a Cas protein or a fusion protein comprising the Cas protein; and (c) a donor sequence comprising a fragment of a wild-type dystrophin gene, wherein the donor sequence comprises exon 52 of the wild-type dystrophin gene.

Clause 3. A CRISPR/Cas-based genome editing system comprising one or more vectors encoding a composition, the composition comprising: (a) a guide RNA (gRNA) targeting a fragment of a mutant dystrophin gene; (b) a Cas protein or a fusion protein comprising the Cas protein; and (c) a donor sequence comprising a fragment of a wild-type dystrophin gene.

Clause 4. A CRISPR/Cas-based genome editing system comprising: (a) a guide RNA (gRNA) targeting a fragment of a mutant dystrophin gene; (b) a Cas protein or a fusion protein comprising the Cas protein; and (c) a donor sequence comprising a fragment of a wild-type dystrophin gene.

Clause 5. The system of clause 3 or 4, wherein the gRNA hybridizes to a target sequence within intron 51 or intron 44 of the mutant dystrophin gene.

Clause 6. The system of clause 1, 2, or 5, wherein the gRNA hybridizes to a target sequence within the polynucleotide sequence of SEQ ID NO: 128 or SEQ ID NO: 156.

Clause 7. The system of any one of clauses 3-6, wherein the donor sequence comprises exon 52 of the wild-type dystrophin gene.

Clause 8. The system of clause 1, 2, or 7, wherein donor sequence comprises the polynucleotide sequence of SEQ ID NO: 53.

Clause 9. The system of any one of clauses 1-8, wherein the fragment of the wild-type dystrophin gene is flanked on both sides by a gRNA spacer and/or a PAM sequence.

Clause 10. The system of any one of clauses 1-9, wherein the gRNA targets an intron that is between exon 51 and exon 52 of the mutant dystrophin gene.

Clause 11. The system of any one of clauses 1-10, wherein the donor sequence comprises multiple exons of the wild-type dystrophin gene or a functional equivalent thereof.

Clause 12. The system of any one of clauses 1-11, wherein the donor sequence comprises one or more exons selected from exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, exon 72, exon 73, exon 74, exon 75, exon 76, exon 77, exon 78, and exon 79 of the wild-type dystrophin gene or a functional equivalent thereof, or wherein the donor sequence comprises exons 52-79 of the wild-type dystrophin gene or a functional equivalent thereof, or wherein the donor sequence comprises exons 45-79 of the wild-type dystrophin gene or a functional equivalent thereof.

Clause 13. The system of any one of clauses 1-12, wherein exon 52 of the mutant dystrophin gene is mutated or at least partially deleted from the dystrophin gene, or wherein exon 52 of the mutant dystrophin gene is deleted and the intron is juxtaposed to where the deleted exon 52 would be in a corresponding wild-type dystrophin gene.

Clause 14. The system of any one of clauses 1-13, wherein the gRNA binds and targets a polynucleotide sequence comprising: (a) a sequence selected from SEQ ID NOs: 29-51, 87, 157-170; (b) a fragment of a sequence selected from SEQ ID NOs: 29-51, 87, 157-170; (c) a complement of a sequence selected from SEQ ID NOs: 29-51, 87, 157-170, or a fragment thereof; (d) a nucleic acid that is substantially identical to a sequence selected from SEQ ID NOs: 29-51, 87, 157-170, or a complement thereof; or (e) a nucleic acid that hybridizes under stringent conditions to a sequence selected from SEQ ID NOs: 29-51, 87, 157-170, or a complement thereof, or a sequence substantially identical thereto.

Clause 15. The system of any one of clauses 1-14, wherein the gRNA binds and targets or is encoded by a polynucleotide sequence selected from SEQ ID NOs: 29-51, 87, 157-170, a complement thereof, or a variant thereof.

Clause 16. The system of any one of clauses 9-15, wherein the gRNA spacer comprises a sequence selected from SEQ ID NOs: 29-51, 87, 157-170, a complement thereof, or a variant thereof.

Clause 17. The system of any one of clauses 1-16, wherein the gRNA comprises a polynucleotide sequence selected from SEQ ID NOs: 64-86, 88, 171-184, a complement thereof, or a variant thereof.

Clause 18. The system of any one of clauses 1-17, wherein the gRNA binds or is encoded by a polynucleotide sequence selected from SEQ ID NOs: 35, 40, and 44, or wherein the gRNA comprises a polynucleotide sequence selected from SEQ ID NOs: 70, 75, and 79.

Clause 19. The system of any one of clauses 1-18, wherein the donor sequence comprises a polynucleotide sequence selected from SEQ ID NOs: 53-56, 154, and 155.

Clause 20. The system of clause 19, wherein the donor sequence comprises a polynucleotide of SEQ ID NO: 55.

Clause 21. The system of clause 19, wherein the donor sequence comprises a polynucleotide of SEQ ID NO: 56.

Clause 22. The system of any one of clauses 1-21, wherein the Cas protein is a Streptococcus pyogenes Cas9 protein or a Staphylococcus aureus Cas9 protein.

Clause 23. The system of any one of clauses 1-22, wherein the Cas protein comprises an amino acid sequence of SEQ ID NO: 18 or SEQ ID NO: 19.

Clause 24. The system of any one of clauses 1, 3, and 5-23, wherein the vector is a viral vector.

Clause 25. The system of clause 24, wherein the vector is an Adeno-associated virus (AAV) vector.

Clause 26. The system of clause 25, wherein the AAV vector is AAV1, AAV2, AAV3, AAV4, AAV5, AAV6, AAV7, AAV8, AAV9, AAV-10, AAV-11, AAV-12, AAV-13, or AAVrh.74 vector.

Clause 27. The system of clause 26, wherein one of the one or more vectors comprises a polynucleotide sequence selected from SEQ ID NOs: 57-60 and 129-130.

Clause 28. The system of any one of clauses 1-27, wherein the molar ratio between gRNA and donor sequence is 1:1, or 1:5, or from 5:1 to 1:10, or from 1:1 to 1:5.

Clause 29. A recombinant polynucleotide encoding a donor sequence, wherein the donor sequence is flanked on both sides by a gRNA spacer and/or a PAM sequence, and wherein the donor sequence comprises one or more exons selected from exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, exon 72, exon 73, exon 74, exon 75, exon 76, exon 77, exon 78, and exon 79 of a dystrophin gene.

Clause 30. The system of any one of clauses 1-28 or the recombinant polynucleotide of clause 29, wherein the dystrophin gene is a human dystrophin gene.

Clause 31. The system or the recombinant polynucleotide of clause 30, wherein the system results in a dystrophin gene that encodes an in-frame transcript comprising an exon 51 joined with an exon comprising a sequence of SEQ ID NO: 53 or SEQ ID NO: 55, and with an intron therebetween.

Clause 32. The system or the recombinant polynucleotide of clause 30 or 31, wherein the donor sequence comprises a polynucleotide sequence comprising exons 52-79 of the human dystrophin gene.

Clause 33. The system or the recombinant polynucleotide of clause 32, wherein the donor sequence comprises the polynucleotide sequence of SEQ ID NO: 55 or SEQ ID NO: 56.

Clause 34. The recombinant polynucleotide of clause 29, wherein the recombinant polynucleotide comprises a sequence selected from SEQ ID NOs: 57-60.

Clause 35. A vector comprising the recombinant polynucleotide of any one of clauses 27-32.

Clause 36. A cell comprising the recombinant polynucleotide of any one of clauses 29-34 or the vector of clause 35.

Clause 37. A composition for restoring dystrophin function in a cell having a mutant dystrophin gene, the composition comprising the system of any one of clauses 1-28 or 30-33, the recombinant polynucleotide of any one of clauses 29-34, or the vector of clause 35.

Clause 38. A kit comprising the system of any one of clauses 1-28 or 30-33, the recombinant polynucleotide of any one of clauses 29-34, or the vector of clause 35, or the composition of clause 35.

Clause 39. A method for restoring dystrophin function in a cell or a subject having a mutant dystrophin gene, the method comprising contacting the cell or the subject with the system of any one of clauses 1-28 or 30-33, the recombinant polynucleotide of any one of clauses 29-34, or the vector of clause 35, or the composition of clause 37.

Clause 40. The method of clause 39, wherein the method results in a dystrophin gene that encodes an in-frame transcript comprising an exon 51 joined with an exon comprising a sequence of SEQ ID NO: 53 or SEQ ID NO: 55, and with an intron therebetween.

Clause 41. A method for restoring dystrophin function in a cell or a subject having a disrupted dystrophin gene caused by one or more deleted or mutated exons, the method comprising contacting the cell or the subject with the system of any one of clauses 1-28 or 30-33, the recombinant polynucleotide of any one of clauses 29-34, or the vector of clause 35, or the composition of clause 37.

Clause 42. The method of clause 41, wherein the method results in a dystrophin gene that encodes an in-frame transcript comprising an exon 51 joined with an exon comprising a sequence of SEQ ID NO: 53 or SEQ ID NO: 55, and with an intron therebetween.

Clause 43. The method of clause 41 or 42, wherein dystrophin function is restored by inserting one or more wild-type exons of dystrophin gene corresponding to the one or more deleted or mutated exons.

Clause 44. The method of any one of clauses 39-43, wherein the subject is suffering from Duchenne Muscular Dystrophy.

Clause 45. A genome editing system for correcting a dystrophin gene, the system comprising a donor sequence comprising exons 52-79 or exons 45-79 of the wild-type dystrophin gene.

Clause 46. The genome editing system of clause 45, further comprising a nuclease selected from homing endonuclease, zinc finger nuclease, TALEN, and Cas protein.

SEQUENCES

SEQ ID NO: 1
NRG (N can be any nucleotide residue, e.g., any of A, G, C, or T)
SEQ ID NO: 2
NGG (N can be any nucleotide residue, e.g., any of A, G, C, or T)
SEQ ID NO: 3
NAG (N can be any nucleotide residue, e.g., any of A, G, C, or T)
SEQ ID NO: 4
NGGNG (N can be any nucleotide residue, e.g., any of A, G, C, or T)
SEQ ID NO: 5
NNAGAAW (W = A or T; N can be any nucleotide residue, e.g., any of A, G, C, or T)
SEQ ID NO: 6
NAAR (R = A or G; N can be any nucleotide residue, e.g., any of A, G, C, or T)
SEQ ID NO: 7
NNGRR (R = A or G; N can be any nucleotide residue, e.g., any of A, G, C, or T)
SEQ ID NO: 8
NNGRRN (R = A or G; N can be any nucleotide residue, e.g., any of A, G, C, or T)
SEQ ID NO: 9
NNGRRT (R = A or G; N can be any nucleotide residue, e.g., any of A, G, C, or T)
SEQ ID NO: 10
NNGRRV (R = A or G; N can be any nucleotide residue, e.g., any of A, G, C, or T)
SEQ ID NO: 11
NNNNGATT (N can be any nucleotide residue, e.g., any of A, G, C, or T)
SEQ ID NO: 12
NNNNGNNN (N can be any nucleotide residue, e.g., any of A, G, C, or T)
SEQ ID NO: 13
NGA (N can be any nucleotide residue, e.g., any of A, G, C, or T)
SEQ ID NO: 14
NNNRRT (R = A or G; N can be any nucleotide residue, e.g., any of A, G, C, or T)
SEQ ID NO: 15
ATTCCT
SEQ ID NO: 16
NGAN (N can be any nucleotide residue, e.g., any of A, G, C, or T)
SEQ ID NO: 17
NGNG (N can be any nucleotide residue, e.g., any of A, G, C, or T)
SEQ ID NO: 18
Streptccoccus pyogenes Cas9
MDKKYSTGLDTGTNSVGWAVITDEYKVPSKKFKVLGNTDRHSIKKNLTGALLFDSGETAEATRLKRTA
RRRYTRRKNRTCYLQEIFSNEMAKVDDSFFHRLEESFLVEEDKKHERHPIFGNIVDEVAYHEKYPTTY
HLRKKLVDSTDKADLRLIYLALAHMIKFRGHFLIEGDLNPDNSDVDKLFIQLVQTYNQLFEENPINAS
GVDAKAILSARLSKSRRLENLTAQLPGEKKNGLFGNLTALSLGLTPNFKSNFDLAEDAKLQLSKDTYD
DDLDNLLAQTGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDEYPFLKDNREKIEKILTFRIPYYVGPLARGNSREAWMTRKSEETTTPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRENASLGTYHDLLKIIKDKDFLDNEEN
EDILEDIVLTLTLFEDREMIEERLKTYAHLFDDKVMKQLKRRRYTGWGRLSRKLINGIRDKQSGKTTL
DELKSDGFANRNEMQLIHDDSLTFKEDIQKAQVSGQGDSLHEHTANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKEDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MTAKSEQETGKATAKYFFYSNIMNFFKTEITLANGEIRKRPLIETNGETGEIVWDKGRDFATVRKVLS
MPQVNIVKKTEVQTGGFSKESILPKRNSDKLTARKKDWDPKKYGGFDSPTVAYSVLVVAKVEKGKSKK
LKSVKELLGITTMERSSFEKNPIDELEAKGYKEVKKDLIIKLPKYSLFELENGRKRMLASAGELQKGN
AYNKHRDKPIREQAENIIHLFTLTNLGAPAAFKYFDTTTDRKRYTSTKEVLDATLIHQSITGLYETRI
DLSQLGGD
SEQ ID NO: 19
Staphylococcus aureus Cas9 molecule
MKRNYILGLDTGITSVGYGIIDYETRDVIDAGVRLFKEANVENNEGRRSKRGARRLKRRRRHRIQRVK
KLLFDYNLLTDHSELSGINPYEARVKGLSQKLSEEEFSAALLHLAKRRGVHNVNEVEEDTGNELSTKE
QISRNSKALEEKYVAELQLERLKKDGEVRGSINREKTSDYVKEAKQLLKVQKAYHQLDQSFIDTYIDL
LETRRTYYEGPGEGSPFGWKDIKEWYEMLMGHCTYFPEELRSVKYAYNADLYNALNDLNNLVITRDEN
EKLEYYEKEQIIENVEKQKKKPTLKQTAKEILVNEEDIKGYRVTSTGKPEFTNLKVYHDIKDITARKE
IIENAELLDQTAKILTTYQSSEDIQEELTNLNSELTQEEIEQISNLKGYTGTHNLSLKAINLILDELW
HTNDNQTAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLEAIPLE
DLLNNPFNYEVDHIIPRSVSFDNSFNNKVLVKQEENSKKGNRTPFQYLSSSDSKISYETFKKHILNLA
KGKGRISKTKKEYLLEERDINRFSVQKDFINRNLVDTRYATRGLMNLLRSYFRVNNLDVKVKSINGGF
TSFLRRKWKFKKERNKGYKHHAEDALITANADFIFKEWKKLDKAKKVMENQMFEEKQAESMPEIETEQ
EYKEIFITPHQIKHIKDFKDYKYSHRVDKKPNRELINDTLYSTRKDDKGNTLIVNNLNGLYDKDNDKL
KKLINKSPEKLLMYHHDPQTYQKLKLIMEQYGDEKNPLYKYYEETGNYLTKYSKKDNGPVIKKIKYYG
NKLNAHLDITDDYPNSRNKVVKLSLKPYRFDVYLDNGVYKFVTVKNLDVIKKENYYEVNSKCYEEAKK
LKKISNQAEFTASFYNNDLIKINGELYRVTGVNNDLLNRIEVNMIDITYREYLENMNDKRPPRIIKTT
ASKTQSIKKYSTDILGNLYEVKSKKHPQIIKKG
SEQ ID NO: 20
codon optimized polynucleotide encoding S. pyogenes Cas9
atggataaaa agtacagcat cgggctggac atcggtacaa actcagtggg gtgggccgtg
attacggacg agtacaaggt accctccaaa aaatttaaag tgctgggtaa cacggacaga
cactctataa agaaaaatct tattggagcc ttgctgttcg actcaggcga gacagccgaa
gccacaaggt tgaagcggac cgccaggagg cggtatacca ggagaaagaa ccgcatatgc
tacctgcaag aaatcttcag taacgagatg gcaaaggttg acgatagctt tttccatcgc
ctggaagaat cctttcttgt tgaggaagac aagaagcacg aacggcaccc catctttggc
aatattgtcg acgaagtggc atatcacgaa aagtacccga ctatctacca cctcaggaag
aagctggtgg actctaccga taaggcggac ctcagactta tttatttggc actcgcccac
atgattaaat ttagaggaca tttcttgatc gagggcgacc tgaacccgga caacagtgac
gtcgataagc tgttcatcca acttgtgcag acctacaatc aactgttcga agaaaaccct
ataaatgctt caggagtcga cgctaaagca atcctgtccg cgcgcctctc aaaatctaga
agacttgaga atctgattgc tcagttgccc ggggaaaaga aaaatggatt gtttggcaac
ctgatcgccc tcagtctcgg actgacccca aatttcaaaa gtaacttcga cctggccgaa
gacgctaagc tccagctgtc caaggacaca tacgatgacg acctcgacaa tctgctggcc
cagattgggg atcagtacgc cgatctcttt ttggcagcaa agaacctgtc cgacgccatc
ctgttgagcg atatcttgag agtgaacacc gaaattacta aagcacccct tagcgcatct
atgatcaagc ggtacgacga gcatcatcag gatctgaccc tgctgaaggc tcttgtgagg
caacagctcc ccgaaaaata caaggaaatc ttctttgacc agagcaaaaa cggctacgct
ggctatatag atggtggggc cagtcaggag gaattctata aattcatcaa gcccattctc
gagaaaatgg acggcacaga ggagttgctg gtcaaactta acagggagga cctgctgcgg
aagcagcgga cctttgacaa cgggtctatc ccccaccaga ttcatctggg cgaactgcac
gcaatcctga ggaggcagga ggatttttat ccttttctta aagataaccg cgagaaaata
gaaaagattc ttacattcag gatcccgtac tacgtgggac ctctcgcccg gggcaattca
cggtttgcct ggatgacaag gaagtcagag gagactatta caccttggaa cttcgaagaa
gtggtggaca agggtgcatc tgcccagtct ttcatcgagc ggatgacaaa ttttgacaag
aacctcccta atgagaaggt gctgcccaaa cattctctgc tctacgagta ctttaccgtc
tacaatgaac tgactaaagt caagtacgtc accgagggaa tgaggaagcc ggcattcctt
agtggagaac agaagaaggc gattgtagac ctgttgttca agaccaacag gaaggtgact
gtgaagcaac ttaaagaaga ctactttaag aagatcgaat gttttgacag tgtggaaatt
tcaggggttg aagaccgctt caatgcgtca ttggggactt accatgatct tctcaagatc
ataaaggaca aagacttcct ggacaacgaa gaaaatgagg atattctcga agacatcgtc
ctcaccctga ccctgttcga agacagggaa atgatagaag agcgcttgaa aacctatgcc
cacctcttcg acgataaagt tatgaagcag ctgaagcgca ggagatacac aggatgggga
agattgtcaa ggaagctgat caatggaatt agggataaac agagtggcaa gaccatactg
gatttcctca aatctgatgg cttcgccaat aggaacttca tgcaactgat tcacgatgac
tctcttacct tcaaggagga cattcaaaag gctcaggtga gcgggcaggg agactccctt
catgaacaca tcgcgaattt ggcaggttcc cccgctatta aaaagggcat ccttcaaact
gtcaaggtgg tggatgaatt ggtcaaggta atgggcagac ataagccaga aaatattgtg
atcgagatgg cccgcgaaaa ccagaccaca cagaagggcc agaaaaatag tagagagcgg
atgaagagga tcgaggaggg catcaaagag ctgggatctc agattctcaa agaacacccc
gtagaaaaca cacagctgca gaacgaaaaa ttgtacttgt actatctgca gaacggcaga
gacatgtacg tcgaccaaga acttgatatt aatagactgt ccgactatga cgtagaccat
atcgtgcccc agtccttcct gaaggacgac tccattgata acaaagtctt gacaagaagc
gacaagaaca ggggtaaaag tgataatgtg cctagcgagg aggtggtgaa aaaaatgaag
aactactggc gacagctgct taatgcaaag ctcattacac aacggaagtt cgataatctg
acgaaagcag agagaggtgg cttgtctgag ttggacaagg cagggtttat taagcggcag
ctggtggaaa ctaggcagat cacaaagcac gtggcgcaga ttttggacag ccggatgaac
acaaaatacg acgaaaatga taaactgata cgagaggtca aagttatcac gctgaaaagc
aagctggtgt ccgattttcg gaaagacttc cagttctaca aagttcgcga gattaataac
taccatcatg ctcacgatgc gtacctgaac gctgttgtcg ggaccgcctt gataaagaag
tacccaaagc tggaatccga gttcgtatac ggggattaca aagtgtacga tgtgaggaaa
atgatagcca agtccgagca ggagattgga aaggccacag ctaagtactt cttttattct
aacatcatga atttttttaa gacggaaatt accctggcca acggagagat cagaaagcgg
ccccttatag agacaaatgg tgaaacaggt gaaatcgtct gggataaggg cagggatttc
gctactgtga ggaaggtgct gagtatgcca caggtaaata tcgtgaaaaa aaccgaagta
cagaccggag gattttccaa ggaaagcatt ttgcctaaaa gaaactcaga caagctcatc
gcccgcaaga aagattggga ccctaagaaa tacgggggat ttgactcacc caccgtagcc
tattctgtgc tggtggtagc taaggtggaa aaaggaaagt ctaagaagct gaagtccgtg
aaggaactct tgggaatcac tatcatggaa agatcatcct ttgaaaagaa ccctatcgat
ttcctggagg ctaagggtta caaggaggtc aagaaagacc tcatcattaa actgccaaaa
tactctctct tcgagctgga aaatggcagg aagagaatgt tggccagcgc cggagagctg
caaaagggaa acgagcttgc tctgccctcc aaatatgtta attttctcta tctcgcttcc
cactatgaaa agctgaaagg gtctcccgaa gataacgagc agaagcagct gttcgtcgaa
cagcacaagc actatctgga tgaaataatc gaacaaataa gcgagttcag caaaagggtt
atcctggcgg atgctaattt ggacaaagta ctgtctgctt ataacaagca ccgggataag
cctattaggg aacaagccga gaatataatt cacctcttta cactcacgaa tctcggagcc
cccgccgcct tcaaatactt tgatacgact atcgaccgga aacggtatac cagtaccaaa
gaggtcctcg atgccaccct catccaccag tcaattactg gcctgtacga aacacggatc
gacctctctc aactgggcgg cgactag
SEQ ID NO: 21
codon optimized nucleic acid sequences encoding S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tcgatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
tccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggatcctatt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta coctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cccattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 22
codon optimized nucleic acid sequences encoding S. aureus Cas9
atgaagcgga actacatcct gggcctggac atcggcatca ccagcgtggg ctacggcatc
atcgactacg agacacggga cgtgatcgat gccggcgtgc ggctgttcaa agaggccaac
gtggaaaaca acgagggcag gcggagcaag agaggcgcca gaaggctgaa gcggcggagg
cggcatagaa tccagagagt gaagaagctg ctgttcgact acaacctgct gaccgaccac
agcgagctga gcggcatcaa cccctacgag gccagagtga agggcctgag ccagaagctg
agcgaggaag agttctctgc cgccctgctg cacctggcca agagaagagg cgtgcacaac
gtgaacgagg tggaagagga caccggcaac gagctgtcca ccaaagagca gatcagccgg
aacagcaagg ccctggaaga gaaatacgtg gccgaactgc agctggaacg gctgaagaaa
gacggcgaag tgcggggcag catcaacaga ttcaagacca gcgactacgt gaaagaagcc
aaacagctgc tgaaggtgca gaaggcctac caccagctgg accagagctt catcgacacc
tacatcgacc tgctggaaac ccggcggacc tactatgagg gacctggcga gggcagcccc
ttcggctgga aggacatcaa agaatggtac gagatgctga tgggccactg cacctacttc
cccgaggaac tgcggagcgt gaagtacgcc tacaacgccg acctgtacaa cgccctgaac
gacctgaaca atctcgtgat caccagggac gagaacgaga agctggaata ttacgagaag
ttccagatca tcgagaacgt gttcaagcag aagaagaagc ccaccctgaa gcagatcgcc
aaagaaatcc tcgtgaacga agaggatatt aagggctaca gagtgaccag caccggcaag
cccgagttca ccaacctgaa ggtgtaccac gacatcaagg acattaccgc ccggaaagag
attattgaga acgccgagct getggatcag attgccaaga tcctgaccat ctaccagagc
agcgaggaca tccaggaaga actgaccaat ctgaactccg agctgaccca ggaagagatc
gagcagatct ctaatctgaa gggctatacc ggcacccaca acctgagcct gaaggccatc
aacctgatcc tggacgagct gtggcacacc aacgacaacc agatcgctat cttcaaccgg
ctgaagctgg tgcccaagaa ggtggacctg tcccagcaga aagagatccc caccaccctg
gtggacgact tcatcctgag ccccgtcgtg aagagaagct tcatccagag catcaaagtg
atcaacgcca tcatcaagaa gtacggcctg cccaacgaca tcattatcga gctggcccgc
gagaagaact ccaaggacgc ccagaaaatg atcaacgaga tgcagaagcg gaaccggcag
accaacgagc ggatcgagga aatcatccgg accaccggca aagagaacgc caagtacctg
atcgagaaga tcaagctgca cgacatgcag gaaggcaagt gcctgtacag cctggaagcc
atccctctgg aagatctgct gaacaacccc ttcaactatg aggtggacca catcatcccc
agaagcgtgt ccttcgacaa cagcttcaac aacaaggtgc tcgtgaagca ggaagaaaac
agcaagaagg gcaaccggac cccattccag tacctgagca gcagcgacag caagatcagc
tacgaaacct tcaagaagca catcctgaat ctggccaagg gcaagggcag aatcagcaag
accaagaaag agtatctgct ggaagaacgg gacatcaaca ggttctccgt gcagaaagac
ttcatcaacc ggaacctggt ggataccaga tacgccacca gaggcctgat gaacctgctg
cggagctact tcagagtgaa caacctggac gtgaaagtga agtccatcaa tggcggcttc
accagctttc tgcggcggaa gtggaagttt aagaaagagc ggaacaaggg gtacaagcac
cacgccgagg acgccctgat cattgccaac gccgatttca tcttcaaaga gtggaagaaa
ctggacaagg ccaaaaaagt gatggaaaac cagatgttcg aggaaaagca ggccgagagc
atgcccgaga tcgaaaccga gcaggagtac aaagagatct tcatcacccc ccaccagatc
aagcacatta aggacttcaa ggactacaag tacagccacc gggtggacaa gaagcctaat
agagagecga ttaacgacac cctgtactcc acccggaagg acgacaaggg caacaccctg
atcgtgaaca atctgaacgg cctgtacgac aaggacaatg acaagctgaa aaagctgatc
aacaagagcc ccgaaaagct gctgatgtac caccacgacc cccagaccta ccagaaactg
aagctgatta tggaacagta cggcgacgag aagaatcccc tgtacaagta ctacgaggaa
accgggaact acctgaccaa gtactccaaa aaggacaacg gccccgtgat caagaagatt
aagtattacg gcaacaaact gaacgcccat ctggacatca ccgacgacta ccccaacagc
agaaacaagg tcgtgaagct gtccctgaag ccctacagat tcgacgtgta cctggacaat
ggcgtgtaca agttcgtgac cgtgaagaat ctggatgtga tcaaaaaaga aaactactac
gaagtgaata gcaagtgcta tgaggaagct aagaagctga agaagatcag caaccaggcc
gagtttatcg cctccttcta caacaacgat ctgatcaaga tcaacggcga gctgtataga
gtgatcggcg tgaacaacga cctgctgaac cggatcgaag tgaacatgat cgacatcacc
taccgcgagt acctggaaaa catgaacgac aagaggcccc ccaggatcat taagacaatc
gectccaaga cccagagcat taagaagtac agcacagaca ttctgggcaa cctgtatgaa
gtgaaatcta agaagcaccc tcagatcatc aaaaagggc
SEQ ID NO: 23
codon optimized nucleic acid sequence encoding S. aureus Cas9
atgaagcgca actacatcct cggactggac atcggcatta cctccgtggg atacggcatc
atcgattacg aaactaggga tgtgatcgac gctggagtca ggctgttcaa agaggcgaac
gtggagaaca acgaggggcg gcgctcaaag aggggggccc gccggctgaa gcgccgccgc
agacatagaa tccagcgcgt gaagaagctg ctgttcgact acaaccttct gaccgaccac
tccgaacttt ccggcatcaa cccatatgag gctagagtga agggattgtc ccaaaagctg
tccgaggaag agttctccgc cgcgttgctc cacctcgcca agcgcagggg agtgcacaat
gtgaacgaag tggaagaaga taccggaaac gagctgtcca ccaaggagca gatcagccgg
aactccaagg ccctggaaga gaaatacgtg gcggaactgc aactggagcg gctgaagaaa
gacggagaag tgcgcggctc gatcaaccgc ttcaagacct cggactacgt gaaggaggcc
aagcagctcc tgaaagtgca aaaggcctat caccaacttg accagtcctt tatcgatacc
tacatcgatc tgctcgagac tcggcggact tactacgagg gtccagggga gggctcccca
tttggttgga aggatattaa ggagtggtac gaaatgctga tgggacactg cacatacttc
cctgaggagc tgcggagcgt gaaatacgca tacaacgcag acctgtacaa cgcgctgaac
gacctgaaca atctcgtgat cacccgggac gagaacgaaa agctcgagta ttacgaaaag
ttccagatta ttgagaacgt gttcaaacag aagaagaagc cgacactgaa gcagattgcc
aaggaaatcc tcgtgaacga agaggacatc aagggctatc gagtgacctc aacgggaaag
ccggagttca ccaatctgaa ggtctaccac gacatcaaag acattaccgc ccggaaggag
atcattgaga acgcggagct gttggaccag attgcgaaga ttctgaccat ctaccaatcc
tccgaggata ttcaggaaga actcaccaac ctcaacagcg aactgaccca ggaggagata
gagcaaatct ccaacctgaa gggctacacc ggaactcata acctgagcct gaaggccatc
aacttgatcc tggacgagct gtggcacacc aacgataacc agatcgctat tttcaatcgg
ctgaagctgg tccccaagaa agtggacctc tcacaacaaa aggagatccc tactaccctt
gtggacgatt tcattctgtc ccccgtggtc aagagaagct tcatacagtc aatcaaagtg
atcaatgcca ttatcaagaa atacggtctg cocaacgaca ttatcattga gctcgcccgc
gagaagaact cgaaggacgc ccagaagatg attaacgaaa tgcagaagag gaaccgacag
actaacgaac ggatcgaaga aatcatccgg accaccggga aggaaaacgc gaagtacctg
atcgaaaaga tcaagctcca tgacatgcag gaaggaaagt gtctgtactc gctggaggcc
attccgctgg aggacttgct gaacaaccct tttaactacg aagtggatca tatcattccg
aggagcgtgt cattcgacaa ttccttcaac aacaaggtcc tcgtgaagca ggaggaaaac
tcgaagaagg gaaaccgcac gccgttccag tacctgagca gcagcgactc caagatttcc
tacgaaacct tcaagaagca catcctcaac ctggcaaagg ggaagggtcg catctccaag
accaagaagg aatatctgct ggaagaaaga gacatcaaca gattctccgt gcaaaaggac
ttcatcaace gcaacctcgt ggatactaga tacgctacte ggggtctgat gaacctcctg
agaagctact ttagagtgaa caatctggac gtgaaggtca agtcgattaa cggaggtttc
acctccttcc tgcggcgcaa gtggaagttc aagaaggaac ggaacaaggg ctacaagcac
cacgccgagg acgccctgat cattgccaac gccgacttca tcttcaaaga atggaagaaa
cttgacaagg ctaagaaggt catggaaaac cagatgttcg aagaaaagca ggccgagtct
atgcctgaaa tcgagactga acaggagtac aaggaaatct ttattacgcc acaccagatc
aaacacatca aggatttcaa ggattacaag tactcacatc gcgtggacaa aaagccgaac
agggaactga tcaacgacac cctctactcc acccggaagg atgacaaagg gaataccctc
atcgtcaaca accttaacgg cctgtacgac aaggacaacg ataagctgaa gaagctcatt
aacaagtcgc ccgaaaagtt gctgatgtac caccacgacc ctcagactta ccagaagctc
aagctgatca tggagcagta tggggacgag aaaaacccgt tgtacaagta ctacgaagaa
actgggaatt atctgactaa gtactccaag aaagataacg gccccgtgat taagaagatt
aagtactacg gcaacaagct gaacgcccat ctggacatca ccgatgacta ccctaattcc
cgcaacaagg tcgtcaagct gagcctcaag ccctaccggt ttgatgtgta ccttgacaat
ggagtgtaca agttcgtgac tctgaagaac cttgacgtga tcaagaagga gaactactac
gaagtcaact ccaagtgcta cgaggaagca aagaagttga agaagatctc gaaccaggcc
gagttcattg cctccttcta taacaacgac ctgattaaga tcaacggcga actgtaccgc
gtcattggcg tgaacaacga tctcctgaac cgcatcgaag tgaacatgat cgacatcact
taccgggaat acctggagaa tatgaacgac aagcgcccgc cccggatcat taagactatc
gcctcaaaga cccagtcgat caagaagtac agcaccgaca tcctgggcaa cctgtacgag
gtcaaatcga agaagcaccc ccagatcatc aagaaggga
SEQ ID NO: 24
codon optimized nucleic acid sequence encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccagagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaggcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 25
codon optimized nucleic acid sequence encoding S. aureus Cas9
accggtgcca ccatgtaccc atacgatgtt ccagattacg cttcgccgaa gaaaaagcgc
aaggtcgaag cgtccatgaa aaggaactac attctggggc tggacatcgg gattacaagc
gtggggtatg ggattattga ctatgaaaca agggacgtga tcgacgcagg cgtcagactg
ttcaaggagg ccaacgtgga aaacaatgag ggacggagaa gcaagagggg agccaggcgc
ctgaaacgac ggagaaggca cagaatccag agggtgaaga aactgctgtt cgattacaac
ctgctgaccg accattctga gctgagtgga attaatcctt atgaagccag ggtgaaaggc
ctgagtcaga agctgtcaga ggaagagttt tccgcagctc tgctgcacct ggctaagcgc
cgaggagtgc ataacgtcaa tgaggtggaa gaggacaccg gcaacgagct gtctacaaag
gaacagatct cacgcaatag caaagctctg gaagagaagt atgtcgcaga gctgcagctg
gaacggctga agaaagatgg cgaggtgaga gggtcaatta ataggttcaa gacaagcgac
tacgtcaaag aagccaagca gctgctgaaa gtgcagaagg cttaccacca gctggatcag
agcttcatcg atacttatat cgacctgctg gagactcgga gaacctacta tgagggacca
ggagaaggga gccccttcgg atggaaagac atcaaggaat ggtacgagat gctgatggga
cattgcacct attttccaga agagctgaga agcgtcaagt acgcttataa cgcagatct
tacaacgccc tgaatgacct gaacaacctg gtcatcacca gggatgaaaa cgagaaactg
gaatactatg agaagttcca gatcatcgaa aacgtgttta agcagaagaa aaagcctaca
ctgaaacaga ttgctaagga gatcctggtc aacgaagagg acatcaaggg ctaccgggtg
acaagcactg gaaaaccaga gttcaccaat ctgaaagtgt atcacgatat taaggacatc
acagcacgga aagaaatcat tgagaacgcc gaactgctgg atcagattgc taagatcctg
actatctacc agagctccga ggacatccag gaagagctga ctaacctgaa cagcgagctg
acccaggaag agatcgaaca gattagtaat ctgaaggggt acaccggaac acacaacctg
tccctgaaag ctatcaatct gattctggat gagctgtggc atacaaacga caatcagatt
gcaatcttta accggctgaa gctggtccca aaaaaggtgg acctgagtca gcagaaagag
atcccaacca cactggtgga cgatttcatt ctgtcacccg tggtcaagcg gagcttcatc
cagagcatca aagtgatcaa cgccatcatc aagaagtacg gcctgcccaa tgatatcatt
atcgagctgg ctagggagaa gaacagcaag gacgcacaga agatgatcaa tgagatgcag
aaacgaaacc ggcagaccaa tgaacgcatt gaagagatta tccgaactac cgggaaagag
aacgcaaagt acctgattga aaaaatcaag ctgcacgata tgcaggaggg aaagtgtctg
tattctctgg aggccatccc cctggaggac ctgctgaaca atccattcaa ctacgaggtc
gatcatatta tccccagaag cgtgtccttc gacaattcct ttaacaacaa ggtgctggtc
aagcaggaag agaactctaa aaagggcaat aggactcctt tccagtacct gtctagttca
gattccaaga tctcttacga aacctttaaa aagcacattc tgaatctggc caaaggaaag
ggccgcatca gcaagaccaa aaaggagtac ctgctggaag agcgggacat caacagattc
tccgtccaga aggattttat taaccggaat ctggtggaca caagatacgc tactcgcggc
ctgatgaatc tgctgcgatc ctatttccgg gtgaacaatc tggatgtgaa agtcaagtcc
atcaacggcg ggttcacatc ttttctgagg cgcaaatgga agtttaaaaa ggagcgcaac
aaagggtaca agcaccatgc cgaagatgct ctgattatcg caaatgccga cttcatcttt
aaggagtgga aaaagctgga caaagccaag aaagtgatgg agaaccagat gttcgaagag
aagcaggccg aatctatgcc cgaaatcgag acagaacagg agtacaagga gattttcatc
actcctcacc agatcaagca tatcaaggat ttcaaggact acaagtactc tcaccgggtg
gataaaaagc ccaacagaga gctgatcaat gacaccctgt atagtacaag aaaagacgat
aaggggaata ccctgattgt gaacaatctg aacggactgt acgacaaaga taatgacaag
ctgaaaaagc tgatcaacaa aagtcccgag aagctgctga tgtaccacca tgatcctcag
acatatcaga aactgaagct gattatggag cagtacggcg acgagaagaa cccactgtat
aagtactatg aagagactgg gaactacctg accaagtata gcaaaaagga taatggcccc
gtgatcaaga agatcaagta ctatgggaac aagctgaatg cccatctgga catcacagac
gattacccta acagtcgcaa caaggtggtc aagctgtcac tgaagccata cagattcgat
gtctatctgg acaacggcgt gtataaattt gtgactgtca agaatctgga tgtcatcaaa
aaggagaact actatgaagt gaatagcaag tgctacgaag aggctaaaaa gctgaaaaag
attagcaacc aggcagagtt catcgcctcc ttttacaaca acgacctgat taagatcaat
ggcgaactgt atagggtcat cggggtgaac aatgatctgc tgaaccgcat tgaagtgaat
atgattgaca tcacttaccg agagtatctg gaaaacatga atgataagcg cccccctcga
attatcaaaa caattgccto taagactcag agtatcaaaa agtactcaac cgacattctg
ggaaacctgt atgaggtgaa gagcaaaaag caccctcaga ttatcaaaaa gggctaagaa
ttc
SEQ ID NO: 26
codon optimized nucleic acid sequences encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 27
codon optimized nucleic acid sequences encoding S. aureus Cas9
aagcggaactacatcctgggcctggacatcggcatcaccagcgtgggctacggcatcatcgactacga
gacacgggacgtgatcgatgccggcgtgcggctgttcaaagaggccaacgtggaaaacaacgagggca
ggcggagcaagagaggcgccagaaggetgaagcggcggaggcggcatagaatccagagagtgaagaag
ctgctgttcgactacaacctgctgaccgaccacagcgagctgagcggcatcaacccctacgaggccag
agtgaagggcctgagccagaagctgagcgaggaagagttctctgccgccctgctgcacctggccaaga
gaagaggcgtgcacaacgtgaacgaggtggaagaggacaccggcaacgagctgtccaccaaagagcag
atcagccggaacagcaaggccctggaagagaaatacgtggccgaactgcagctggaacggctgaagaa
agacggcgaagtgcggggcagcatcaacagattcaagaccagcgactacgtgaaagaagccaaacagc
tgctgaaggtgcagaaggcctaccaccagctggaccagagcttcatcgacacctacatcgacctgctg
gaaacccggcggacctactatgagggacctggcgagggcagccccttcggctggaaggacatcaaaga
atggtacgagatgctgatgggccactgcacctacttccccgaggaactgcggagcgtgaagtacgcct
acaacgccgacctgtacaacgccctgaacgacctgaacaatctcgtgatcaccagggacgagaacgag
aagctggaatattacgagaagttccagatcatcgagaacgtgttcaagcagaagaagaagcccaccct
gaagcagatcgccaaagaaatcctcgtgaacgaagaggatattaagggctacagagtgaccagcaccg
gcaagcccgagttcaccaacctgaaggtgtaccacgacatcaaggacattaccgcccggaaagagatt
attgagaacgccgagctgctggatcagattgccaagatcctgaccatctaccagagcagcgaggacat
ccaggaagaactgaccaatctgaactccgagctgacccaggaagagatcgagcagatctctaatctga
agggctataccggcacccacaacctgagcctgaaggccatcaacctgatcctggacgagctgtggcac
accaacgacaaccagatcgctatcttcaaccggctgaagctggtgcccaagaaggtggacctgtccca
gcagaaagagatccccaccaccctggtggacgacttcatcctgagccccgtcgtgaagagaagcttca
tccagagcatcaaagtgatcaacgccatcatcaagaagtacggcctgcccaacgacatcattatcgag
ctggcccgcgagaagaactccaaggacgcccagaaaatgatcaacgagatgcagaagcggaaccggca
gaccaacgagcggatcgaggaaatcatccggaccaccggcaaagagaacgccaagtacctgatcgaga
agatcaagctgcacgacatgcaggaaggcaagtgcctgtacagcctggaagccatccctctggaagat
ctgctgaacaaccccttcaactatgaggtggaccacatcatccccagaagcgtgtccttcgacaacag
cttcaacaacaaggtgctcgtgaagcaggaagaaaacagcaagaagggcaaccggaccccattccagt
acctgagcagcagcgacagcaagatcagctacgaaaccttcaagaagcacatcctgaatctggccaag
ggcaagggcagaatcagcaagaccaagaaagagtatctgctggaagaacgggacatcaacaggttctc
cgtgcagaaagacttcatcaaccggaacctggtggataccagatacgccaccagaggcctgatgaacc
tgctgcggagctacttcagagtgaacaacctggacgtgaaagtgaagtccatcaatggcggcttcacc
agctttctgcggcggaagtggaagtttaagaaagagcggaacaaggggtacaagcaccacgccgagga
cgccctgatcattgccaacgccgatttcatcttcaaagagtggaagaaactggacaaggccaaaaaag
tgatggaaaaccagatgttcgaggaaaagcaggccgagagcatgcccgagatcgaaaccgagcaggag
tacaaagagatcttcatcaccccccaccagatcaagcacattaaggacttcaaggactacaagtacag
ccaccgggtggacaagaagcctaatagagagctgattaacgacaccctgtactccacccggaaggacg
acaagggcaacaccetgatcgtgaacaatctgaacggcctgtacgacaaggacaatgacaagctgaaa
aagctgatcaacaagagccccgaaaagctgctgatgtaccaccacgacccccagacctaccagaaact
gaagctgattatggaacagtacggcgacgagaagaatcccctgtacaagtactacgaggaaaccggga
actacctgaccaagtactccaaaaaggacaacggccccgtgatcaagaagattaagtattacggcaac
aaactgaacgcccatctggacatcaccgacgactaccccaacagcagaaacaaggtcgtgaagctgtc
cctgaagccctacagattcgacgtgtacctggacaatggcgtgtacaagttcgtgaccgtgaagaatc
tggatgtgatcaaaaaagaaaactactacgaagtgaatagcaagtgctatgaggaagctaagaagctg
aagaagatcagcaaccaggccgagtttatcgcctccttctacaacaacgatctgatcaagatcaacgg
cgagctgtatagagtgatcggcgtgaacaacgacctgctgaaccggatcgaagtgaacatgatcgaca
tcacctaccgcgagtacctggaaaacatgaacgacaagaggccccccaggatcattaagacaatcgcc
tccaagacccagagcattaagaagtacagcacagacattctgggcaacctgtatgaagtgaaatctaa
gaagcaccctcagatcatcaaaaagggc
SEQ ID NO: 28
Vector (pDO242) encoding codon optimized nucleic acid sequence encoding S. aureus
Cas9
ctaaattgtaagcgttaatattttgttaaaattcgcgttaaatttttgttaaatcagctcatttttta
accaataggccgaaatcggcaaaatcccttataaatcaaaagaatagaccgagatagggttgagtgtt
gttccagtttggaacaagagtccactattaaagaacgtggactccaacgtcaaagggcgaaaaaccgt
ctatcagggcgatggcccactacgtgaaccatcaccctaatcaagttttttggggtcgaggtgccgta
aagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgct
gcgcgtaaccaccacacccgccgcgcttaatgcgccgctacagggcgcgtcccattcgccattcaggc
tgcgcaactgttgggaagggcgatcggtgcgggcctcttcgctattacgccagctggcgaaaggggga
tgtgctgcaaggcgattaagttgggtaacgccagggttttcccagtcacgacgttgtaaaacgacggc
cagtgagcgcgcgtaatacgactcactatagggcgaattgggtacCtttaattctagtactatgcaTg
cgttgacattgattattgactagttattaatagtaatcaattacggggtcattagttcatagcccata
tatggagttccgcgttacataacttacggtaaatggcccgcctggctgaccgcccaacgacccccgcc
cattgacgtcaataatgacgtatgttcccatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattatgcccagtacatgaccttatgggactttc
ctacttggcagtacatctacgtattagtcatcgctattaccatggtgatgcggttttggcagtacatc
aatgggcgtggatagcggtttgactcacggggatttccaagtctccaccccattgacgtcaatgggag
tttgttttggcaccaaaatcaacgggactttccaaaatgtcgtaacaactccgccccattgacgcaaa
tgggcggtaggcgtgtacggtgggaggtctatataagcagagctctctggctaactaccggtgccacc
ATGAAAAGGAACTACATTCTGGGGCTGGACATCGGGATTACAAGCGTGGGGTATGGGATTATTGACTA
TGAAACAAGGGACGTGATCGACGCAGGCGTCAGACTGTTCAAGGAGGCCAACGTGGAAAACAATGAGG
GACGGAGAAGCAAGAGGGGAGCCAGGCGCCTGAAACGACGGAGAAGGCACAGAATCCAGAGGGTGAAG
AAACTGCTGTTCGATTACAACCTGCTGACCGACCATTCTGAGCTGAGTGGAATTAATCCTTATGAAGC
CAGGGTGAAAGGCCTGAGTCAGAAGCTGTCAGAGGAAGAGTTTTCCGCAGCTCTGCTGCACCTGGCTA
AGCGCCGAGGAGTGCATAACGTCAATGAGGTGGAAGAGGACACCGGCAACGAGCTGTCTACAAAGGAA
CAGATCTCACGCAATAGCAAAGCTCTGGAAGAGAAGTATGTCGCAGAGCTGCAGCTGGAACGGCTGAA
GAAAGATGGCGAGGTGAGAGGGTCAATTAATAGGTTCAAGACAAGCGACTACGTCAAAGAAGCCAAGC
AGCTGCTGAAAGTGCAGAAGGCTTACCACCAGCTGGATCAGAGCTTCATCGATACTTATATCGACCTG
CTGGAGACTCGGAGAACCTACTATGAGGGACCAGGAGAAGGGAGCCCCTTCGGATGGAAAGACATCAA
GGAATGGTACGAGATGOTGATGGGACATTGCACCTATTTTCCAGAAGAGCTGAGAAGCGTCAAGTACG
CTTATAACGCAGATCTGTACAACGCCCTGAATGACCTGAACAACCTGGTCATCACCAGGGATGAAAAC
GAGAAACTGGAATACTATGAGAAGTTCCAGATCATCGAAAACGTGTTTAAGCAGAAGAAAAAGCCTAC
ACTGAAACAGATTGCTAAGGAGATCCTGGTCAACGAAGAGGACATCAAGGGCTACCGGGTGACAAGCA
CTGGAAAACCAGAGTTCACCAATCTGAAAGTGTATCACGATATTAAGGACATCACAGCACGGAAAGAA
ATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATC
TGAAGGGGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGACCTGAG
TCAGCAGAAAGAGATCCCAACCACACTGGTGGACGATTTCATTCTGTCACCCGTGGTCAAGCGGAGCT
TCATCCAGAGCATCAAAGTGATCAACGCCATCATCAAGAAGTACGGCCTGCCCAATGATATCATTATC
GAGCTGGCTAGGGAGAAGAACAGCAAGGACGCACAGAAGATGATCAATGAGATGCAGAAACGAAACCG
GCAGACCAATGAACGCATTGAAGAGATTATCCGAACTACCGGGAAAGAGAACGCAAAGTACCTGATTG
AAAAAATCAAGCTGCACGATATGCAGGAGGGAAAGTGTCTGTATTCTCTGGAGGCCATCCCCCTGGAG
GACCTGCTGAACAATCCATTCAACTACGAGGTCGATCATATTATCCCCAGAAGCGTGTCCTTCGACAA
TTCCTTTAACAACAAGGTGCTGGTCAAGCAGGAAGAGAACTCTAAAAAGGGCAATAGGACTCCTTTCC
AGTACCTGTCTAGTTCAGATTCCAAGATCTCTTACGAAACCTTTAAAAAGCACATTCTGAATCTGGCC
AAAGGAAAGGGCCGCATCAGCAAGACCAAAAAGGAGTACCTGCTGGAAGAGCGGGACATCAACAGATT
CTCCGTCCAGAAGGATTTTATTAACCGGAATCTGGTGGACACAAGATACGCTACTCGCGGCCTGATGA
ATCTGCTGCGATCCTATTTCCGGGTGAACAATCTGGATGTGAAAGTCAAGTCCATCAACGGCGGGTTC
ACATCTTTTCTGAGGCGCAAATGGAAGTTTAAAAAGGAGCGCAACAAAGGGTACAAGCACCATGCCGA
AGATGCTCTGATTATCGCAAATGCCGACTTCATCTTTAAGGAGTGGAAAAAGCTGGACAAAGCCAAGA
AAGTGATGGAGAACCAGATGTTCGAAGAGAAGCAGGCCGAATCTATGCCCGAAATCGAGACAGAACAG
GAGTACAAGGAGATTTTCATCACTCCTCACCAGATCAAGCATATCAAGGATTTCAAGGACTACAAGTA
CTCTCACCGGGTGGATAAAAAGCCCAACAGAGAGCTGATCAATGACACCCTGTATAGTACAAGAAAAG
ACGATAAGGGGAATACCCTGATTGTGAACAATCTGAACGGACTGTACGACAAAGATAATGACAAGCTG
AAAAAGCTGATCAACAAAAGTCCCGAGAAGCTGCTGATGTACCACCATGATCCTCAGACATATCAGAA
ACTGAAGCTGATTATGGAGCAGTACGGCGACGAGAAGAACCCACTGTATAAGTACTATGAAGAGACTG
GGAACTACCTGACCAAGTATAGCAAAAAGGATAATGGCCCCGTGATCAAGAAGATCAAGTACTATGGG
AACAAGOTGAATGCCCATCTGGACATCACAGACGATTACCCTAACAGTCGCAACAAGGTGGTCAAGCT
GTCACTGAAGCCATACAGATTCGATGTCTATCTGGACAACGGCGTGTATAAATTTGTGACTGTCAAGA
ATCTGGATGTCATCAAAAAGGAGAACTACTATGAAGTGAATAGCAAGTGCTACGAAGAGGCTAAAAAG
CTGAAAAAGATTAGCAACCAGGCAGAGTTCATCGCCTCCTTTTACAACAACGACCTGATTAAGATCAA
TGGCGAACTGTATAGGGTCATCGGGGTGAACAATGATCTGCTGAACCGCATTGAAGTGAATATGATTG
ACATCACTTACCGAGAGTATCTGGAAAACATGAATGATAAGCGCCCCCCTCGAATTATCAAAACAATT
GCCTCTAAGACTCAGAGTATCAAAAAGTACTCAACCGACATTCTGGGAAACCTGTATGAGGTGAAGAG
CAAAAAGCACCCTCAGATTATCAAAAAGGGCagcggaggcaagcgtcctgctgctactaagaaagctg
gtcaagctaagaaaaagaaaggatcctacccatacgatgttccagattacgcttaagaattcctagag
ctcgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtggggtggggcaggacagcaagggggaggattgggaag
agaatagcaggcatgctggggaggtagcggccgcCCgcggtggagctccagcttttgttccctttagt
gagggttaattgcgcgcttggcgtaatcatggtcatagctgtttcctgtgtgaaattgttatccgctc
acaattccacacaacatacgagccggaagcataaagtgtaaagcctggggtgcctaatgagtgagcta
actcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
cctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaaggacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
aagaagatcctttgatcttttctacggggtctgacgctcagtggaacgaaaactcacgttaagggatt
ttggtcatgagattatcaaaaaggatcttcacctagatccttttaaattaaaaatgaagttttaaatc
aatctaaagtatatatgagtaaacttggtctgacagttaccaatgettaatcagtgaggcacctatct
cagcgatctgtctatttcgttcatccatagttgcctgactccccgtcgtgtagataactacgatacgg
gagggcttaccatctggccccagtgctgcaatgataccgcgagacccacgctcaccggctccagattt
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatggttatggcagcactgcataattct
cttactgtcatgccatccgtaagatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcggggcgaaaactctcaaggatcttaccgctg
ttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtctcatgagc
ggatacatatttgaatgtatttagaaaaataaacaaataggggttccgcgcacatttccccgaaaagt
gccac

| | | | | |
|---|---|---|---|---|
| | Target/ | | Spacer Sequence | |
| Guide # | Original gRNA | Cas9 | (Sequence the gRNA targets and binds) | nt |

First Round Screening:

| | | | | |
|---|---|---|---|---|
| gAP1 | Dyst (Intron51) | SaCas9 | CTTTACTTTGTATTATGTAAA | 21 |
| | | | (SEQ ID NO: 29) | |
| gAP2 | Dyst (Intron51) | SaCas9 | TTTGAAATATTTTTGATATCT | 21 |
| | | | (SEQ ID NO: 30) | |
| gAP3 | Dyst (Intron51) | SaCas9 | TTTAAGTAATCCGAGGTACTC | 21 |
| | | | (SEQ ID NO: 31) | |
| gAP4 | Dyst (Intron51) | SaCas9 | TTTAAATACATTGTCGTAATT | 21 |
| | | | (SEQ ID NO: 32) | |
| 9AP5 | Dyst (Intron51) | SaCas9 | TACCTTAATTTTGACGTCACA | 21 |
| | | | (SEQ ID NO: 33) | |
| gAP6 | Dyst (Intron51) | SaCas9 | ATTTGACAGGTGAGAAATCTC | 21 |
| | | | (SEQ ID NO: 34) | |
| gAP7 | Dyst (Intron51) | SaCas9 | TCATTTATAATACAGGGGAAT | 21 |
| | | | (SEQ ID NO: 35) | |
| gAP8 | Dyst (Intron51) | SaCas9 | TTAAAGTCATTTATAATACAG | 21 |
| | | | (SEQ ID NO: 36) | |
| gAP9 | Dyst (Intron51) | SaCas9 | AAATAGACACTGAAGAAAGGG | 21 |
| | | | (SEQ ID NO: 37) | |
| gAP10 | Dyst (Intron51) | SaCas9 | CCCCAATTAAAATAAAATTTA | 21 |
| | | | (SEQ ID NO: 38) | |

Second Round Screening:

| | | | | |
|---|---|---|---|---|
| gAP11 | g3 | SaCas9 | TAAGTAATCCGAGGTACTC | 19 |
| | | | (SEQ ID NO: 39) | |
| gAP12 | g3 | SaCas9 | TTAAGTAATCCGAGGTACTC | 20 |
| | | | SEQ ID NO: 40) | |
| gAP13 | g3 | SaCas9 | GTTTAAGTAATCCGAGGTACTC | 22 |
| | | | (SEQ ID NO: 41) | |
| gAP14 | g3 | SaCas9 | GGTTTAAGTAATCCGAGGTACTC | 23 |
| | | | (SEQ ID NO: 42) | |
| gAP15 | g6 | SaCas9 | TTGACAGGTGAGAAATCTC | 19 |
| | | | (SEQ ID NO: 43) | |
| gAP16 | g6 | SaCas9 | TTTGACAGGTGAGAAATCTC | 20 |
| | | | (SEQ ID NO: 44) | |
| gAP17 | g6 | SaCas9 | CATTTGACAGGTGAGAAATCTC | 22 |
| | | | (SEQ ID NO: 45) | |
| gAP18 | g6 | SaCas9 | TCATTTGACAGGTGAGAAATCTC | 23 |
| | | | (SEQ ID NO: 46) | |
| gAP19 | g7 | SaCas9 | ATTTATAATACAGGGGAAT | 19 |
| | | | (SEQ ID NO: 47) | |
| gAP20 | g7 | SaCas9 | CATTTATAATACAGGGGAAT | 20 |
| | | | (SEQ ID NO: 48) | |
| gAP21 | g7 | SaCas9 | GTCATTTATAATACAGGGGAAT | 22 |
| | | | (SEQ ID NO: 49) | |
| gAP22 | g7 | SaCas9 | AGTCATTTATAATACAGGGGAAT | 23 |
| | | | (SEQ ID NO: 50) | |
| gAP23 | scrambled | SaCas9 | GCACTACCAGAGCTAACTCA | 20 |
| | | | (SEQ ID NO: 51) | |

SEQ ID NO: 52
SaCas9 gRNA scaffold
GTTTTAGTACTCTGGAAACAGAATCTACTAAAACAAGGCAAAATGCCGTGTTTATCTCGTCAACTTGT
TGGCGAGA
SEQ ID NO: 53
Exon 52
GCAACAATGCAGGATTTGGAACAGAGGCGTCCCCAGTTGGAAGAACTCATTACCGCTGCCCAAAATTT
GAAAAACAAGACCAGCAATCAAGAGGCTAGAACAATCATTACGGATCGAA
SEQ ID NO: 54
Donor sequence comprising exon 52
GTTAAATTGTTTTCTATAAACCCTTATACAGTAACATCTTTTTTATTTCTAAAAGTGTTTTGGCTGGT
CTCACAATTGTACTTTACTTTGTATTATGTAAAAGGAATACACAACGOTGAAGAACCCTGATACTAAG
GGATATTTGTTCTTACAGGCAACAATGCAGGATTTGGAACAGAGGCGTCCCCAGTTGGAAGAACTCAT
TACCGCTGCCCAAAATTTGAAAAACAAGACCAGCAATCAAGAGGCTAGAACAATCATTACGGATCGAA
GTAAGTTTTTTAACAAGCATGGGACACACAAAGCAAGATGCATGACAAGTTTCAATAAAAACTTAAGT
TCATATATCCCCCTCACATTTATAAAAATAATGTGAAATAATTGTAAATGATAACAATTGTGCTGAGA
TTTTCAGTCCATAATGTTACCTTTTAATAAATGAATGTAATTCCATTGAATAGAAGAAATAC
SEQ ID NO: 55
Super exon (exons 52-79)
GCAACAATGCAGGATTTGGAACAGAGGCGTCCCCAGTTGGAAGAACTCATTACCGCTGCCCAAAATTT
GAAAAACAAGACCAGCAATCAAGAGGCTAGAACAATCATTACGGATCGAATTGAAAGAATTCAGAATC
AGTGGGATGAAGTACAAGAACACCTTCAGAACCGGAGGCAACAGTTGAATGAAATGTTAAAGGATTCA
ACACAATGGCTGGAAGCTAAGGAAGAAGCTGAGCAGGTCTTAGGACAGGCCAGAGCCAAGCTTGAGTC
ATGGAAGGAGGGTCCCTATACAGTAGATGCAATCCAAAAGAAAATCACAGAAACCAAGCAGTTGGCCA
AAGACCTCCGCCAGTGGCAGACAAATGTAGATGTGGCAAATGACTTGGCCCTGAAACTTCTCCGGGAT
TATTCTGCAGATGATACCAGAAAAGTCCACATGATAACAGAGAATATCAATGCCTCTTGGAGAAGCAT
TCATAAAAGGGTGAGTGAGCGAGAGGCTGCTTTGGAAGAAACTCATAGATTACTGCAACAGTTCCCCC
TGGACCTGGAAAAGTTTCTTGCCTGGCTTACAGAAGCTGAAACAACTGCCAATGTCCTACAGGATGCT
ACCCGTAAGGAAAGGCTCCTAGAAGACTCCAAGGGAGTAAAAGAGCTGATGAAACAATGGCAAGACCT
CCAAGGTGAAATTGAAGCTCACACAGATGTTTATCACAACCTGGATGAAAACAGCCAAAAAATCCTGA
GATCCCTGGAAGGTTCCGATGATGCAGTCCTGTTACAAAGACGTTTGGATAACATGAACTTCAAGTGG
AGTGAACTTCGGAAAAAGTCTCTCAACATTAGGTCCCATTTGGAAGCCAGTTCTGACCAGTGGAAGCG
TCTGCACCTTTCTCTGCAGGAACTTCTGGTGTGGCTACAGCTGAAAGATGATGAATTAAGCCGGCAGG
CACCTATTGGAGGCGACTTTCCAGCAGTTCAGAAGCAGAACGATGTACATAGGGCCTTCAAGAGGGAA
TTGAAAACTAAAGAACCTGTAATCATGAGTACTCTTGAGACTGTACGAATATTTCTGACAGAGCAGCC
TTTGGAAGGACTAGAGAAACTCTACCAGGAGCCCAGAGAGCTGCCTCCTGAGGAGAGAGCCCAGAATG
TCACTCGGCTTCTACGAAAGCAGGCTGAGGAGGTCAATACTGAGTGGGAAAAATTGAACCTGCACTCC
GCTGACTGGCAGAGAAAAATAGATGAGACCCTTGAAAGACTCCAGGAACTTCAAGAGGCCACGGATGA
GCTGGACCTCAAGCTGCGCCAAGCTGAGGTGATCAAGGGATCCTGGCAGCCCGTGGGCGATCTCCTCA
TTGACTCTCTCCAAGATCACCTCGAGAAAGTCAAGGCACTTCGAGGAGAAATTGCGCCTCTGAAAGAG
AACGTGAGCCACGTCAATGACCTTGCTCGCCAGCTTACCACTTTGGGCATTCAGCTCTCACCGTATAA
CCTCAGCACTCTGGAAGACCTGAACACCAGATGGAAGCTTCTGCAGGTGGCCGTCGAGGACCGAGTCA
GGCAGCTGCATGAAGCCCACAGGGACTTTGGTCCAGCATCTCAGCACTTTCTTTCCACGTCTGTCCAG
GGTCCCTGGGAGAGAGCCATCTCGCCAAACAAAGTGCCCTACTATATCAACCACGAGACTCAAACAAC
TTGCTGGGACCATCCCAAAATGACAGAGCTCTACCAGTCTTTAGCTGACCTGAATAATGTCAGATTCT
CAGCTTATAGGACTGCCATGAAACTCCGAAGACTGCAGAAGGCCCTTTGCTTGGATCTCTTGAGCCTG
TCAGCTGCATGTGATGCCTTGGACCAGCACAACCTCAAGCAAAATGACCAGCCCATGGATATCCTGCA
GATTATTAATTGTTTGACCACTATTTATGACCGCCTGGAGCAAGAGCACAACAATTTGGTCAACGTCC
CTCTCTGCGTGGATATGTGTCTGAACTGGCTGCTGAATGTTTATGATACGGGACGAACAGGGAGGATC
CGTGTCCTGTCTTTTAAAACTGGCATCATTTCCCTGTGTAAAGCACATTTGGAAGACAAGTACAGATA
CCTTTTCAAGCAAGTGGCAAGTTCAACAGGATTTTGTGACCAGCGCAGGCTGGGCCTCCTTCTGCATG
ATTCTATCCAAATTCCAAGACAGTTGGGTGAAGTTGCATCCTTTGGGGGCAGTAACATTGAGCCAAGT
GTCCGGAGCTGCTTCCAATTTGCTAATAATAAGCCAGAGATCGAAGCGGCCCTCTTCCTAGACTGGAT
GAGACTGGAACCCCAGTCCATGGTGTGGCTGCCCGTCCTGCACAGAGTGGCTGCTGCAGAAACTGCCA
AGCATCAGGCCAAATGTAACATCTGCAAAGAGTGTCCAATCATTGGATTCAGGTACAGGAGTCTAAAG
CACTTTAATTATGACATCTGCCAAAGCTGCTTTTTTTCTGGTCGAGTTGCAAAAGGCCATAAAATGCA
CTATCCCATGGTGGAATATTGCACTCCGACTACATCAGGAGAAGATGTTCGAGACTTTGCCAAGGTAC
TAAAAAACAAATTTCGAACCAAAAGGTATTTTGCGAAGCATCCCCGAATGGGCTACCTGCCAGTGCAG
ACTGTCTTAGAGGGGGACAACATGGAAACTCCCGTTACTCTGATCAACTTCTGGCCAGTAGATTCTGC
GCCTGCCTCGTCCCCTCAGCTTTCACACGATGATACTCATTCACGCATTGAACATTATGCTAGCAGGC
TAGCAGAAATGGAAAACAGCAATGGATCTTATCTAAATGATAGCATCTCTCCTAATGAGAGCATAGAT
GATGAACATTTGTTAATCCAGCATTACTGCCAAAGTTTGAACCAGGACTCCCCCCTGAGCCAGCCTCG
TAGTCCTGCCCAGATCTTGATTTCCTTAGAGAGTGAGGAAAGAGGGGAGCTAGAGAGAATCCTAGCAG
ATCTTGAGGAAGAAAACAGGAATCTGCAAGCAGAATATGACCGTCTAAAGCAGCAGCACGAACATAAA
GGCCTGTCCCCACTGCCGTCCCCTCCTGAAATGATGCCCACCTCTCCCCAGAGTCCCCGGGATGCTGA
GCTCATTGCTGAGGCCAAGCTACTGCGTCAACACAAAGGCCGCCTGGAAGCCAGGATGCAAATCCTGG
AAGACCACAATAAACAGCTGGAGTCACAGTTACACAGGCTAAGGCAGCTGCTGGAGCAACCCCAGGCA
GAGGCCAAAGTGAATGGCACAACGGTGTCCTCTCCTTCTACCTCTCTACAGAGGTCCGACAGCAGTCA
GCCTATGCTGCTCCGAGTGGTTGGCAGTCAAACTTCGGACTCCATGGGTGAGGAAGATCTTCTCAGTC
CTCCCCAGGACACAAGCACAGGGTTAGAGGAGGTGATGGAGCAACTCAACAACTCCTTCCCTAGTTCA
AGAGGAAGAAATACCOCTGGAAAGCCAATGAGAGAGGACACAATGTAG
SEQ ID NO: 56
Donor sequence including super exon (exons 52-79)
gTTAAtTTGcgTTCTAgccACCgagATACAGTAACATCTTTTTTATTTCTAAAAGTGTTTTGGCTGGT
CTCACAATTGTACTTTACTTTGTATTATGTAAAAGGAATACACAACGOTGAAGAACCCTGATACTAAG
GGATATTTGTTCTTACAGGCAACAATGCAGGATTTGGAACAGAGGCGTCCCCAGTTGGAAGAACTCAT
TACCGCTGCCCAAAATTTGAAAAACAAGACCAGCAATCAAGAGGCTAGAACAATCATTACGGATCGAA
TTGAAAGAATTCAGAATCAGTGGGATGAAGTACAAGAACACCTTCAGAACCGGAGGCAACAGTTGAAT
GAAATGTTAAAGGATTCAACACAATGGCTGGAAGCTAAGGAAGAAGCTGAGCAGGTCTTAGGACAGGC
CAGAGCCAAGCTTGAGTCATGGAAGGAGGGTCCCTATACAGTAGATGCAATCCAAAAGAAAATCACAG
AAACCAAGCAGTTGGCCAAAGACCTCCGCCAGTGGCAGACAAATGTAGATGTGGCAAATGACTTGGCC
CTGAAACTTCTCCGGGATTATTCTGCAGATGATACCAGAAAAGTCCACATGATAACAGAGAATATCAA
TGCCTCTTGGAGAAGCATTCATAAAAGGGTGAGTGAGCGAGAGGCTGCTTTGGAAGAAACTCATAGAT
TACTGCAACAGTTCCCCCTGGACCTGGAAAAGTTTCTTGCCTGGCTTACAGAAGCTGAAACAACTGCC
AATGTCCTACAGGATGCTACCCGTAAGGAAAGGCTCCTAGAAGACTCCAAGGGAGTAAAAGAGCTGAT
GAAACAATGGCAAGACCTCCAAGGTGAAATTGAAGCTCACACAGATGTTTATCACAACCTGGATGAAA
ACAGCCAAAAAATCCTGAGATCCCTGGAAGGTTCCGATGATGCAGTCCTGTTACAAAGACGTTTGGAT
AACATGAACTTCAAGTGGAGTGAACTTCGGAAAAAGTCTCTCAACATTAGGTCCCATTTGGAAGCCAG
TTCTGACCAGTGGAAGCGTCTGCACCTTTCTCTGCAGGAACTTCTGGTGTGGCTACAGCTGAAAGATG
ATGAATTAAGCCGGCAGGCACCTATTGGAGGCGACTTTCCAGCAGTTCAGAAGCAGAACGATGTACAT
AGGGCCTTCAAGAGGGAATTGAAAACTAAAGAACCTGTAATCATGAGTACTCTTGAGACTGTACGAAT
ATTTCTGACAGAGCAGCCTTTGGAAGGACTAGAGAAACTCTACCAGGAGCCCAGAGAGCTGCCTCCTG
AGGAGAGAGCCCAGAATGTCACTCGGCTTCTACGAAAGCAGGCTGAGGAGGTCAATACTGAGTGGGAA
AAATTGAACCTGCACTCCGCTGACTGGCAGAGAAAAATAGATGAGACCCTTGAAAGACTCCAGGAACT
TCAAGAGGCCACGGATGAGCTGGACCTCAAGCTGCGCCAAGCTGAGGTGATCAAGGGATCCTGGCAGC
CCGTGGGCGATCTCCTCATTGACTCTCTCCAAGATCACCTCGAGAAAGTCAAGGCACTTCGAGGAGAA
ATTGCGCCTCTGAAAGAGAACGTGAGCCACGTCAATGACCTTGCTCGCCAGCTTACCACTTTGGGCAT
TCAGCTCTCACCGTATAACCTCAGCACTCTGGAAGACCTGAACACCAGATGGAAGCTTCTGCAGGTGG
CCGTCGAGGACCGAGTCAGGCAGCTGCATGAAGCCCACAGGGACTTTGGTCCAGCATCTCAGCACTTT
CTTTCCACGTCTGTCCAGGGTCCCTGGGAGAGAGCCATCTCGCCAAACAAAGTGCCCTACTATATCAA
CCACGAGACTCAAACAACTTGCTGGGACCATCCCAAAATGACAGAGCTCTACCAGTCTTTAGCTGACC
TGAATAATGTCAGATTCTCAGCTTATAGGACTGCCATGAAACTCCGAAGACTGCAGAAGGCCCTTTGC
TTGGATCTCTTGAGCCTGTCAGCTGCATGTGATGCCTTGGACCAGCACAACCTCAAGCAAAATGACCA
GCCCATGGATATCCTGCAGATTATTAATTGTTTGACCACTATTTATGACCGCCTGGAGCAAGAGCACA
ACAATTTGGTCAACGTCCCTCTCTGCGTGGATATGTGTCTGAACTGGCTGCTGAATGTTTATGATACG
GGACGAACAGGGAGGATCCGTGTCCTGTCTTTTAAAACTGGCATCATTTCCCTGTGTAAAGCACATTT
GGAAGACAAGTACAGATACCTTTTCAAGCAAGTGGCAAGTTCAACAGGATTTTGTGACCAGCGCAGGC
TGGGCCTCCTTCTGCATGATTCTATCCAAATTCCAAGACAGTTGGGTGAAGTTGCATCCTTTGGGGGC
AGTAACATTGAGCCAAGTGTCCGGAGCTGCTTCCAATTTGCTAATAATAAGCCAGAGATCGAAGCGGC
CCTCTTCCTAGACTGGATGAGACTGGAACCCCAGTCCATGGTGTGGCTGCCCGTCCTGCACAGAGTGG
CTGCTGCAGAAACTGCCAAGCATCAGGCCAAATGTAACATCTGCAAAGAGTGTCCAATCATTGGATTC
AGGTACAGGAGTCTAAAGCACTTTAATTATGACATCTGCCAAAGCTGCTTTTTTTCTGGTCGAGTTGC
AAAAGGCCATAAAATGCACTATCCCATGGTGGAATATTGCACTCCGACTACATCAGGAGAAGATGTTC
GAGACTTTGCCAAGGTACTAAAAAACAAATTTCGAACCAAAAGGTATTTTGCGAAGCATCCCCGAATG
GGCTACCTGCCAGTGCAGACTGTCTTAGAGGGGGACAACATGGAAACTCCCGTTACTCTGATCAACTT
CTGGCCAGTAGATTCTGCGCCTGCCTCGTCCCCTCAGCTTTCACACGATGATACTCATTCACGCATTG
AACATTATGCTAGCAGGCTAGCAGAAATGGAAAACAGCAATGGATCTTATCTAAATGATAGCATCTCT
CCTAATGAGAGCATAGATGATGAACATTTGTTAATCCAGCATTACTGCCAAAGTTTGAACCAGGACTC
CCCCCTGAGCCAGCCTCGTAGTCCTGCCCAGATCTTGATTTCCTTAGAGAGTGAGGAAAGAGGGGAGC
TAGAGAGAATCCTAGCAGATOTTGAGGAAGAAAACAGGAATCTGCAAGCAGAATATGACCGTCTAAAG
CAGCAGCACGAACATAAAGGCCTGTCCCCACTGCCGTCCCCTCCTGAAATGATGCCCACCTCTCCCCA
GAGTCCCCGGGATGCTGAGCTCATTGCTGAGGCCAAGCTACTGCGTCAACACAAAGGCCGCCTGGAAG
CCAGGATGCAAATCCTGGAAGACCACAATAAACAGCTGGAGTCACAGTTACACAGGCTAAGGCAGCTG
CTGGAGCAACCCCAGGCAGAGGCCAAAGTGAATGGCACAACGGTGTCCTCTCCTTCTACCTCTCTACA
GAGGTCCGACAGCAGTCAGCCTATGCTGCTCCGAGTGGTTGGCAGTCAAACTTCGGACTCCATGGGTG
AGGAAGATCTTCTCAGTCCTCCCCAGGACACAAGCACAGGGTTAGAGGAGGTGATGGAGCAACTCAAC
AACTCCTTCCCTAGTTCAAGAGGAAGAAATACCCCTGGAAAGCCAATGAGAGAGGACACAATGTAGTC
GTTTAAACCGCTGATCAGCCTCGAAACTTGTTTATTGCAGCTTATAATGGTTACAAATAAAGCAATAG
CATCACAAATTTCACAAATAAAGCATTTTTTTCACTGCATTCTAGTTGTGGTTTGTCCAAACTCATCA
ATGTATCTTAGTAAGTTTTTTAACAAGCATGGGACACACAAAGCAAGATGCATGACAAGTTTCAATAA
AAACTTAAGTTCATATATCCCCCTCACATTTATAAAAATAATGTGAAATAATTGTAAATGATAACAAT
TGTGCTGAGATTTTCAGTCCATAATGTTACCTTTTAATAAATGAATGTAATTCCATTGAATAGAAGAA
ATAC
SEQ ID NO: 57
AAV expression cassette for version 1 exon52 donor sequence with gRNA7:
AAV ITR, U6 PROMOTER, [symbol] PAM (ATTCCT), SaCas9 gRNA
SCAFFOLD, donor sequence, exon52
TTGGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAGAGAGGGAGTGGCCA
ACTCCATCACTAGGGGTTCCTCAGATCTGAATTCGGTACCTTCCTAGGGCCTATTTCCCATG
ATTCCTTCATATTTGCATATACGATACAAGGCTGTTAGAGAGATAATTGGAATTAATTTGAC
TGTAAACACAAAGATATTAGTACAAAATACGTGACGTAGAAAGTAATAATTTCTTGGGTAGT
TTGCAGTTTTAAAATTATGTTTTAAAATGGACTATCATATGCTTACCGTAACTTGAAAGTAT
TTCGATTTCTTGGCTTTATATATCTTGTGGAAAGGACGAAACACCG [symbol] [symbol]
GTTTTAGTACTCTGGAAACAGAATCTACTAAAACAAGGCAAAATGCCGTGTTTATCT
CGTCAACTTGTTGGCGAGATTTTTTTCTAGACCCAGCTTTCTTGTACAAAGTTGGCGTTTAA
AC [symbol] gttaaattgttttctataaacccttatacagta
acatcttttttatttctaaaagtgttttggctggtctcacaattgtactttactttgtatta
tgtaaaaggaatacacaacgctgaagaaccctgatactaagggatatttgttcttacaggca
acaatgcaggatttggaacagaggcgtccccagttggaagaactcattaccgctgcccaaaa
tttgaaaaacaagaccagcaatcaagaggctagaacaatcattacggatcgaagtaagtttt
ttaacaagcatgggacacacaaagcaagatgcatgacaagtttcaataaaaacttaagttca
tatatccccctcacatttataaaaataatgtgaaataattgtaaatgataacaattgtgctg
agattttcagtccataatgttaccttttaataaatgaatgtaattccattgaatagaagaaa
tacATTCCT [symbol] GCTAGCTCGGAGAGACGACATCTAGAATTAAA
CTGTCACTATCGATTACTAATTTTTTGCTCATAATAGAAGCAGCGATTAAAGGAATAGAATC
AACAGTTCCAGTAACATCTCTTAGTGCATACATTTTTTTATCAGCAGGAACAATATCATCTG
ACTGACCTGTGATGCTCATTCCAACTTCATTAATTGTTTTAATGAATTTTTCTTTAGATAAT
TCACTTGTTCAACCTTTACATGACTCTAATTTATCAATAGTACCACCAGTTACTCCAAGTCC
TCTACCAGAAAGTTTACAAACCTTTACTCCATAACTTGCAACTAACGGACTATATACTAAAC
TTGTTTTGTCTCCAACTCCGCCAGTTGAATGCTTATCAGCTTTTAAACCTGTAACCTCACTG
ACATCATAAACATATCCTGATTCAACATAAGATTGGGTTAATGCTAAAGTTTCTGCTTTGGT
CATTCCATTAAAATAAACCGCCATAGCAAAAGCAGCCATTTGATAATCTGTTACATTATTTT
TAACGTAACTGTTTATCAATCATTTAATTTCTTCAGCTGATAATTCTATTGAATGTTTCTTT
TTTTCTATAATTTCACTAAAACTGTAGTTCATAAGTCTCCTTTTGTAAGAGTGCACAATATT
TACACCATTACTCTTTCTACTATATTATAATAGAATAGACATATAAAAAACATAAGGAGTAC
AAATGGTTTTTGATAAAAATAACAAAGTTTATAGTGAATGAATAAATAGCCAAAAATTGGAT
GATGAGTTGAAAAGCCTTTTAGTAAATGCTACTGATGATGAATTGCATGCAGCATTTGAAGG
AATAGAGTTAGAATTTGGAACAGCAGGTATAAGAGGTATTCTTGGAGCAGGACCTGGAAGAT
TTAACGTTTACACTGTTAAAAAAGTTACTATTGCATTTGCAGAATTATTAAAACAAAATTAC
CCAAATAGGTTGAATGATGGAATAGTTGTTGGTCATGATAACCGTCATAATTCTAAACAGTT
TGCAAAAGTTGTAGCCGAAGTTTTATCAAGCTTGTGAAATAGCTGTTGAAGCTGGATTAGAA
TTTGTTAAAACATCAACAGGATTTTCAAAATCAGGTGCAACATTTGAAGATGTTAAACTAAT
GAAGTCAGTTGTTAAAGACAATGOTTTAGTTAAAGCAGCTGGTGGAGTTAGAACATTTGAAG
ATGCTCAAAAAATGATTGAAGCAGGAGCTGACCGCTTAGGAACAAGTGGTGGAGTAGCTATT
ATTAAAGGTGAAGAAAACAACGCGAGTTACTAAAACTAGCGTTTTTTTATTTTGCTCATTTT
TATTAAAAGTTTGCAAAAAGGAACATAAAAATTCTAATTATTGATACTAAAGTTATTAAAAA
GAAGATTTTGGTTGATTTTATAAAGGTCATAGAATATAATATTTTAGCATGTGTATTTTGTG
TGCTCATTTACAACCGTCTCGCGGCCGCGGGGATCCAGACATGATAAGATACATTGATGAGT
TTGGACAAACCACAACTAGAATGCAGTGAAAAAAATGCTTTATTTGTGAAATTTGTGATGCT
ATTGCTTTATTTGTAACCATTATAAGCTGCAATAAACAAGTTAACAACAACAATTGCATTCA
TTTTATGTTTCAGGTTCAGGGGGAGGTGTGGGAGGTTTTTTAGTCGACCTCGAGCAGTGTGG
TTTTGCAAGAGGAAGCAAAAAGCCTCTCCACCCAGGCCTGGAATGTTTCCACCCAAGTCGAA
GGCAGTGTGGTTTTGCAAGAGGAAGCAAAAAGCCTCTCCACCCAGGCCTGGAATGTTTCCAC
CCAATGTCGAGCAACCCCGCCCAGCGTCTTGTCATTGGCGAATTCGAACACGCAGATGCAGT
CGGGGCGGCGCGGTCCCAGGTCCACTTCGCATATTAAGGTGACGCGTGTGGCCTCGAACACC
GAGCGACCCTGCAGCCAATATGGGATCGGCCATTGAACAAGATGGATTGCACGCAGGTTCTC
CGGCCGCTTGGGTGGAGAGGCTATTCGGCTATGACTGGGCACAACAGACAATCGGCTGCTCT
GATGCCGCCGTGTTCCGGCTGTCAGCGCAGGGGCGCCCGGTTCTTTTTGTCAAGACCGACCT
GTCCGGTGCCCTGAATGAACTGCAGGACGAGGCAGCGCGGCTATCGTGGCTGGCCACGACGG
GCGTTCCTTGCGCAGCTGTGCTCGACGTTGTCACTGAAGCGGGAAGGGACTGGCTGCTATTG
GGCGAAGTGCCGGGGCAGGATCTCCTGTCATCTCACCTTGCTCCTGCCGAGAAAGTATCCAT
CATGGCTGATGCAATGCGGCGGCTGCATACGCTTGATCCGGCTACCTGCCCATTCGACCACC
AAGCGAAACATCGCATCGAGCGAGCACGTACTCGGATGGAAGCCGGTCTTGTCGATCAGGAT
GATCTGGACGAAGAGCATCAGGGGCTCGCGCCAGCCGAACTGTTCGCCAGGCTCAAGGCGCG
CATGCCCGACGGCGAGGATCTCGTCGTGACCCATGGCGATGCCTGCTTGCCGAATATCATGG
TGGAAAATGGCCGCTTTTCTGGATTCATCGACTGTGGCCGGCTGGGTGTGGCGGACCGCTAT
CAGGACATAGCGTTGGCTACCCGTGATATTGCTGAAGAGCTTGGCGGCGAATGGGCTGACCG
CTTCCTCGTGCTTTACGGTATCGCCGCTCCCGATTCGCAGCGCATCGCCTTCTATCGCCTTC
TTGACGAGTTCTTCTGAGGGGATCCGTCGACTAGAGCTCGCTGATCAGCCTCGACTGTGCCT
TCTAGTTGCCAGCCATCTGTTGTTTGCCCCTCCCCCGTGCCTTCCTTGACCCTGGAAGGTGC
CACTCCCACTGTCCTTTCCTAATAAAATGAGGAAATTGCATCGCATTGTCTGAGTAGGTGTC
ATTCTATTCTGGGGGGTGGGGTGGGGCAGGACAGCAAGGGGGAGGATTGGGAAGACAATAGC
AGGCATGCTGGGGAGAGATCTAGGAACCCCTAGTGATGGAGTTGGCCACTCCCTCTCTGCGC
GCTCGCTCGCTCACTGAGGCCGCCCGGGCAAAGCCCGGGCGTCGGGCGACCTTTGGTCGCCC
GGCCTCAGTGAGCGAGCGAGCGCGCAGAGAGGGAGTGGCCAA
SEQ ID NO: 58
AAV expression cassette for version 1 exon52 donor sequence with gRNA12:
AAV ITR, U6 PROMOTER, [symbol] PAM, SaCas9 gRNA SCAFFOLD, donor
sequence, exon52
TTGGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAGAGAGGGAGTGGCCA
ACTCCATCACTAGGGGTTCCTCAGATCTGAATTCGGTACCTTCCTAGGGCCTATTTCCCATG
ATTCCTTCATATTTGCATATACGATACAAGGCTGTTAGAGAGATAATTGGAATTAATTTGAC
TGTAAACACAAAGATATTAGTACAAAATACGTGACGTAGAAAGTAATAATTTCTTGGGTAGT
TTGCAGTTTTAAAATTATGTTTTAAAATGGACTATCATATGCTTACCGTAACTTGAAAGTAT
TTCGATTTOTTGGCTTTATATATCTTGTGGAAAGGACGAAACACCG [symbol] [symbol]
GTTTTAGTACTCTGGAAACAGAATCTACTAAAACAAGGCAAAATGCCGTGTTTATCTC
GTCAACTTGTTGGCGAGATTTTTTTCTAGACCCAGCTTTCTTGTACAAAGTTGGCGTTTAAA
C [symbol] gttaaattgttttctataaacccttatacagtaac
atcttttttatttctaaaagtgttttggctggtctcacaattgtactttactttgtattatg
taaaaggaatacacaacgctgaagaaccctgatactaagggatatttgttcttacaggcaac
aatgcaggatttggaacagaggcgtccccagttggaagaactcattaccgctgcccaaaatt
tgaaaaacaagaccagcaatcaagaggctagaacaatcattacggatcgaagtaagtttttt
aacaagcatgggacacacaaagcaagatgcatgacaagtttcaataaaaacttaagttcata
tatccccctcacatttataaaaataatgtgaaataattgtaaatgataacaattgtgctgag
attttcagtccataatgttaccttttaataaatgaatgtaattccattgaatagaagaaata
C [symbol] GCTAGCTCGGAGAGACGACATCTAGAATTAAACTG
TCACTATCGATTACTAATTTTTTGCTCATAATAGAAGCAGCGATTAAAGGAATAGAATCAAC
AGTTCCAGTAACATCTCTTAGTGCATACATTTTTTTATCAGCAGGAACAATATCATCTGACT
GACCTGTGATGCTCATTCCAACTTCATTAATTGTTTTAATGAATTTTTCTTTAGATAATTCA
CTTGTTCAACCTTTACATGACTCTAATTTATCAATAGTACCACCAGTTACTCCAAGTCCTCT
ACCAGAAAGTTTACAAACCTTTACTCCATAACTTGCAACTAACGGACTATATACTAAACTTG
TTTTGTCTCCAACTCCGCCAGTTGAATGCTTATCAGCTTTTAAACCTGTAACCTCACTGACA
TCATAAACATATCCTGATTCAACATAAGATTGGGTTAATGCTAAAGTTTCTGCTTTGGTCAT
TCCATTAAAATAAACCGCCATAGCAAAAGCAGCCATTTGATAATCTGTTACATTATTTTTAA
CGTAACTGTTTATCAATCATTTAATTTCTTCAGCTGATAATTCTATTGAATGTTTCTTTTTT
TCTATAATTTCACTAAAACTGTAGTTCATAAGTCTCCTTTTGTAAGAGTGCACAATATTTAC
ACCATTACTCTTTCTACTATATTATAATAGAATAGACATATAAAAAACATAAGGAGTACAAA
TGGTTTTTGATAAAAATAACAAAGTTTATAGTGAATGAATAAATAGCCAAAAATTGGATGAT
GAGTTGAAAAGCCTTTTAGTAAATGCTACTGATGATGAATTGCATGCAGCATTTGAAGGAAT
AGAGTTAGAATTTGGAACAGCAGGTATAAGAGGTATTCTTGGAGCAGGACCTGGAAGATTTA
ACGTTTACACTGTTAAAAAAGTTACTATTGCATTTGCAGAATTATTAAAACAAAATTACCCA
AATAGGTTGAATGATGGAATAGTTGTTGGTCATGATAACCGTCATAATTCTAAACAGTTTGC
AAAAGTTGTAGCCGAAGTTTTATCAAGCTTGTGAAATAGCTGTTGAAGCTGGATTAGAATTT
GTTAAAACATCAACAGGATTTTCAAAATCAGGTGCAACATTTGAAGATGTTAAACTAATGAA
GTCAGTTGTTAAAGACAATGCTTTAGTTAAAGCAGCTGGTGGAGTTAGAACATTTGAAGATG
CTCAAAAAATGATTGAAGCAGGAGCTGACCGCTTAGGAACAAGTGGTGGAGTAGCTATTATT
AAAGGTGAAGAAAACAACGCGAGTTACTAAAACTAGCGTTTTTTTATTTTGCTCATTTTTAT
TAAAAGTTTGCAAAAAGGAACATAAAAATTCTAATTATTGATACTAAAGTTATTAAAAAGAA
GATTTTGGTTGATTTTATAAAGGTCATAGAATATAATATTTTAGCATGTGTATTTTGTGTGC
TCATTTACAACCGTCTCGCGGCCGCGGGGATCCAGACATGATAAGATACATTGATGAGTTTG
GACAAACCACAACTAGAATGCAGTGAAAAAAATGCTTTATTTGTGAAATTTGTGATGCTATT
GCTTTATTTGTAACCATTATAAGCTGCAATAAACAAGTTAACAACAACAATTGCATTCATTT
TATGTTTCAGGTTCAGGGGGAGGTGTGGGAGGTTTTTTAGTCGACCTCGAGCAGTGTGGTTT
TGCAAGAGGAAGCAAAAAGCCTCTCCACCCAGGCCTGGAATGTTTCCACCCAAGTCGAAGGC
AGTGTGGTTTTGCAAGAGGAAGCAAAAAGCCTCTCCACCCAGGCCTGGAATGTTTCCACCCA
ATGTCGAGCAACCCCGCCCAGCGTCTTGTCATTGGCGAATTCGAACACGCAGATGCAGTCGG
GGCGGCGCGGTCCCAGGTCCACTTCGCATATTAAGGTGACGCGTGTGGCCTCGAACACCGAG
CGACCCTGCAGCCAATATGGGATCGGCCATTGAACAAGATGGATTGCACGCAGGTTCTCCGG
CCGCTTGGGTGGAGAGGCTATTCGGCTATGACTGGGCACAACAGACAATCGGCTGCTCTGAT
GCCGCCGTGTTCCGGCTGTCAGCGCAGGGGCGCCCGGTTCTTTTTGTCAAGACCGACCTGTC
CGGTGCCCTGAATGAACTGCAGGACGAGGCAGCGCGGCTATCGTGGCTGGCCACGACGGGCG
TTCCTTGCGCAGCTGTGCTCGACGTTGTCACTGAAGCGGGAAGGGACTGGCTGCTATTGGGC
GAAGTGCCGGGGCAGGATCTCCTGTCATCTCACCTTGCTCCTGCCGAGAAAGTATCCATCAT
GGCTGATGCAATGCGGCGGCTGCATACGCTTGATCCGGCTACCTGCCCATTCGACCACCAAG
CGAAACATCGCATCGAGCGAGCACGTACTCGGATGGAAGCCGGTCTTGTCGATCAGGATGAT
CTGGACGAAGAGCATCAGGGGCTCGCGCCAGCCGAACTGTTCGCCAGGCTCAAGGCGCGCAT
GCCCGACGGCGAGGATCTCGTCGTGACCCATGGCGATGCCTGCTTGCCGAATATCATGGTGG
AAAATGGCCGCTTTTCTGGATTCATCGACTGTGGCCGGCTGGGTGTGGCGGACCGCTATCAG
GACATAGCGTTGGCTACCCGTGATATTGCTGAAGAGCTTGGCGGCGAATGGGCTGACCGCTT
CCTCGTGCTTTACGGTATCGCCGCTCCCGATTCGCAGCGCATCGCCTTCTATCGCCTTCTTG
ACGAGTTCTTCTGAGGGGATCCGTCGACTAGAGCTCGCTGATCAGCCTCGACTGTGCCTTCT
AGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATT
CTATTCTGGGGGGTGGGGTGGGGCAGGACAGCAAGGGGGAGGATTGGGAAGACAATAGCAGG
CATGCTGGGGAGAGATCTAGGAACCCCTAGTGATGGAGTTGGCCACTCCCTCTCTGCGCGCT
CGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAA
SEQ ID NO: 59
AAV expression cassette for version 2 exon52 donor sequence with gRNA7:
*Note: version 2 is referring to modified donor sequence for improved detection via deep
sequencing analysis (these modified nucleotides are provided in lowercase)
AAV ITR, U6 PROMOTER, [symbol] PAM (ATTCCT), SaCas9 gRNA
SCAFFOLD, donor sequence, exon52
TTGGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAGAGAGGGAGTGGCCA
ACTCCATCACTAGGGGTTCCTCAGATCTGAATTCGGTACCTTCCTAGGGCCTATTTCCCATG
ATTCCTTCATATTTGCATATACGATACAAGGCTGTTAGAGAGATAATTGGAATTAATTTGAC
TGTAAACACAAAGATATTAGTACAAAATACGTGACGTAGAAAGTAATAATTTCTTGGGTAGT
TTGCAGTTTTAAAATTATGTTTTAAAATGGACTATCATATGCTTACCGTAACTTGAAAGTAT
TTCGATTTCTTGGCTTTATATATCTTGTGGAAAGGACGAAACACCG [symbol] [symbol]
GTTTTAGTACTCTGGAAACAGAATCTACTAAAACAAGGCAAAATGCCGTGTTTATCT
CGTCAACTTGTTGGCGAGATTTTTTTCTAGACCCAGCTTTCTTGTACAAAGTTGGCGTTTAA
AC [symbol] gttaatttgcgttctagccaccgagatacagta
acatcttttttatttctaaaagtgttttggctggtctcacaattgtactttactttgtatta
tgtaaaaggaatacacaacgctgaagaaccctgatactaagggatatttgttcttacaggca
acaatgcaggatttggaacagaggcgtccccagttggaagaactcattaccgctgcccaaaa
tttgaaaaacaagaccagcaatcaagaggctagaacaatcattacggatcgaagtaagtttt
ttaacaagcatgggacacacaaagcaagatgcatgacaagtttcaataaaaacttaagttca
tatatccccctcacatttataaaaataatgtgaaataattgtaaatgataacaattgtgctg
agattttcagtccataatgttaccttttaataaatgaatgtaattccattgaatagaagaaa
tac [symbol] GCTAGCTCGGAGAGACGACATCTAGAATTAAA
CTGTCACTATCGATTACTAATTTTTTGCTCATAATAGAAGCAGCGATTAAAGGAATAGAATC
AACAGTTCCAGTAACATCTCTTAGTGCATACATTTTTTTATCAGCAGGAACAATATCATCTG
ACTGACCTGTGATGCTCATTCCAACTTCATTAATTGTTTTAATGAATTTTTCTTTAGATAAT
TCACTTGTTCAACCTTTACATGACTCTAATTTATCAATAGTACCACCAGTTACTCCAAGTCC
TCTACCAGAAAGTTTACAAACCTTTACTCCATAACTTGCAACTAACGGACTATATACTAAAC
TTGTTTTGTCTCCAACTCCGCCAGTTGAATGCTTATCAGCTTTTAAACCTGTAACCTCACTG
ACATCATAAACATATCCTGATTCAACATAAGATTGGGTTAATGCTAAAGTTTCTGCTTTGGT
CATTCCATTAAAATAAACCGCCATAGCAAAAGCAGCCATTTGATAATCTGTTACATTATTTT
TAACGTAACTGTTTATCAATCATTTAATTTCTTCAGCTGATAATTCTATTGAATGTTTCTTT
TTTTCTATAATTTCACTAAAACTGTAGTTCATAAGTCTCCTTTTGTAAGAGTGCACAATATT
TACACCATTACTCTTTCTACTATATTATAATAGAATAGACATATAAAAAACATAAGGAGTAC
AAATGGTTTTTGATAAAAATAACAAAGTTTATAGTGAATGAATAAATAGCCAAAAATTGGAT
GATGAGTTGAAAAGCCTTTTAGTAAATGCTACTGATGATGAATTGCATGCAGCATTTGAAGG
AATAGAGTTAGAATTTGGAACAGCAGGTATAAGAGGTATTCTTGGAGCAGGACCTGGAAGAT
TTAACGTTTACACTGTTAAAAAAGTTACTATTGCATTTGCAGAATTATTAAAACAAAATTAC
CCAAATAGGTTGAATGATGGAATAGTTGTTGGTCATGATAACCGTCATAATTCTAAACAGTT
TGCAAAAGTTGTAGCCGAAGTTTTATCAAGCTTGTGAAATAGCTGTTGAAGCTGGATTAGAA
TTTGTTAAAACATCAACAGGATTTTCAAAATCAGGTGCAACATTTGAAGATGTTAAACTAAT
GAAGTCAGTTGTTAAAGACAATGOTTTAGTTAAAGCAGCTGGTGGAGTTAGAACATTTGAAG
ATGCTCAAAAAATGATTGAAGCAGGAGCTGACCGCTTAGGAACAAGTGGTGGAGTAGCTATT
ATTAAAGGTGAAGAAAACAACGCGAGTTACTAAAACTAGCGTTTTTTTATTTTGCTCATTTT
TATTAAAAGTTTGCAAAAAGGAACATAAAAATTCTAATTATTGATACTAAAGTTATTAAAAA
GAAGATTTTGGTTGATTTTATAAAGGTCATAGAATATAATATTTTAGCATGTGTATTTTGTG
TGCTCATTTACAACCGTCTCGCGGCCGCGGGGATCCAGACATGATAAGATACATTGATGAGT
TTGGACAAACCACAACTAGAATGCAGTGAAAAAAATGCTTTATTTGTGAAATTTGTGATGCT
ATTGCTTTATTTGTAACCATTATAAGCTGCAATAAACAAGITAACAACAACAATTGCATTCA
TTTTATGTTTCAGGTTCAGGGGGAGGTGTGGGAGGTTTTTTAGTCGACCTCGAGCAGTGTGG
TTTTGCAAGAGGAAGCAAAAAGCCTCTCCACCCAGGCCTGGAATGTTTCCACCCAAGTCGAA
GGCAGTGTGGTTTTGCAAGAGGAAGCAAAAAGCCTCTCCACCCAGGCCTGGAATGTTTCCAC
CCAATGTCGAGCAACCCCGCCCAGCGTCTTGTCATTGGCGAATTCGAACACGCAGATGCAGT
CGGGGCGGCGCGGTCCCAGGTCCACTTCGCATATTAAGGTGACGCGTGTGGCCTCGAACACC
GAGCGACCCTGCAGCCAATATGGGATCGGCCATTGAACAAGATGGATTGCACGCAGGTTCTC
CGGCCGCTTGGGTGGAGAGGCTATTCGGCTATGACTGGGCACAACAGACAATCGGCTGCTCT
GATGCCGCCGTGTTCCGGCTGTCAGCGCAGGGGCGCCCGGTTCTTTTTGTCAAGACCGACCT
GTCCGGTGCCCTGAATGAACTGCAGGACGAGGCAGCGCGGCTATCGTGGCTGGCCACGACGG
GCGTTCCTTGCGCAGCTGTGCTCGACGTTGTCACTGAAGCGGGAAGGGACTGGCTGCTATTG
GGCGAAGTGCCGGGGCAGGATCTCCTGTCATCTCACCTTGCTCCTGCCGAGAAAGTATCCAT
CATGGCTGATGCAATGCGGCGGCTGCATACGCTTGATCCGGCTACCTGCCCATTCGACCACC
AAGCGAAACATCGCATCGAGCGAGCACGTACTCGGATGGAAGCCGGTCTTGTCGATCAGGAT
GATCTGGACGAAGAGCATCAGGGGCTCGCGCCAGCCGAACTGTTCGCCAGGCTCAAGGCGCG
CATGCCCGACGGCGAGGATCTCGTCGTGACCCATGGCGATGCCTGCTTGCCGAATATCATGG
TGGAAAATGGCCGCTTTTCTGGATTCATCGACTGTGGCCGGCTGGGTGTGGCGGACCGCTAT
CAGGACATAGCGTTGGCTACCCGTGATATTGCTGAAGAGCTTGGCGGCGAATGGGCTGACCG
CTTCCTCGTGCTTTACGGTATCGCCGCTCCCGATTCGCAGCGCATCGCCTTCTATCGCCTTC
TTGACGAGTTCTTCTGAGGGGATCCGTCGACTAGAGCTCGCTGATCAGCCTCGACTGTGCCT
TCTAGTTGCCAGCCATCTGTTGTTTGCCCCTCCCCCGTGCCTTCCTTGACCCTGGAAGGTGC
CACTCCCACTGTCCTTTCCTAATAAAATGAGGAAATTGCATCGCATTGTCTGAGTAGGTGTC
ATTCTATTCTGGGGGGTGGGGTGGGGCAGGACAGCAAGGGGGAGGATTGGGAAGACAATAGC
AGGCATGCTGGGGAGAGATCTAGGAACCCCTAGTGATGGAGTTGGCCACTCCCTCTCTGCGC
GCTCGCTCGCTCACTGAGGCCGCCCGGGCAAAGCCCGGGCGTCGGGCGACCTTTGGTCGCCC
GGCCTCAGTGAGCGAGCGAGCGCGCAGAGAGGGAGTGGCCAA
SEQ ID NO: 60
AAV expression cassette for superexon (exon52-exon79) donor sequence with gRNA7:
*Note: version 2 is referring to modified donor sequence for improved detection via deep
sequencing analysis (these modified nucleotides are provided in lowercase)
AAV ITR, U6 PROMOTER, [symbol] PAM (AATCCT), SaCas9 gRNA
SCAFFOLD, donor sequence, exon52-79 cDNA sequence, [symbol]
TTGGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAGAGAGGGAGTGGCCA
ACTCCATCACTAGGGGTTCCTCAGATCTGAATTCGGTACCTTCCTAGGGCCTATTTCCCATG
ATTCCTTCATATTTGCATATACGATACAAGGCTGTTAGAGAGATAATTGGAATTAATTTGAC
TGTAAACACAAAGATATTAGTACAAAATACGTGACGTAGAAAGTAATAATTTOTTGGGTAGT
TTGCAGTTTTAAAATTATGTTTTAAAATGGACTATCATATGCTTACCGTAACTTGAAAGTAT
TTCGATTTOTTGGCTTTATATATCTTGTGGAAAGGACGAAACACCG [symbol] [symbol]
GTTTTAGTACTCTGGAAACAGAATCTACTAAAACAAGGCAAAATGCCGTGTTTATCT
CGTCAACTTGTTGGCGAGATTTTTTTCTAGACCCAGCTTTCTTGTACAAAGTTGGCGTTTAA
AC [symbol] gttaatttgcgttctagccaccgagatacagta
acatcttttttatttctaaaagtgttttggctggtctcacaattgtactttactttgtatta
tgtaaaaggaatacacaacgctgaagaaccctgatactaagggatatttgttcttacaggca
acaatgcaggatttggaacagaggcgtccccagttggaagaactcattaccgctgcccaaaa
tttgaaaaacaagaccagcaatcaagaggctagaacaatcattacggatcgaattgaaagaa
ttcagaatcagtgggatgaagtacaagaacaccttcagaaccggaggcaacagttgaatgaa
atgttaaaggattcaacacaatggctggaagctaaggaagaagctgagcaggtcttaggaca
ggccagagccaagcttgagtcatggaaggagggtccctatacagtagatgcaatccaaaaga
aaatcacagaaaccaagcagttggccaaagacctccgccagtggcagacaaatgtagatgtg
gcaaatgacttggccctgaaacttctccgggattattctgcagatgataccagaaaagtcca
catgataacagagaatatcaatgcctcttggagaagcattcataaaagggtgagtgagcgag
aggctgctttggaagaaactcatagattactgcaacagttccccctggacctggaaaagttt
cttgcctggcttacagaagctgaaacaactgccaatgtcctacaggatgctacccgtaagga
aaggctcctagaagactccaagggagtaaaagagctgatgaaacaatggcaagacctccaag
gtgaaattgaagctcacacagatgtttatcacaacctggatgaaaacagccaaaaaatcctg
agatccctggaaggttccgatgatgcagtcctgttacaaagacgtttggataacatgaactt
caagtggagtgaacttcggaaaaagtctctcaacattaggtcccatttggaagccagttctg
accagtggaagcgtctgcacctttctctgcaggaacttctggtgtggctacagctgaaagat
gatgaattaagccggcaggcacctattggaggcgactttccagcagttcagaagcagaacga
tgtacatagggccttcaagagggaattgaaaactaaagaacctgtaatcatgagtactcttg
agactgtacgaatatttctgacagagcagcctttggaaggactagagaaactctaccaggag
cccagagagctgcctcctgaggagagagcccagaatgtcactcggcttctacgaaagcaggc
tgaggaggtcaatactgagtgggaaaaattgaacctgcactccgctgactggcagagaaaaa
tagatgagacccttgaaagactccaggaacttcaagaggccacggatgagctggacctcaag
ctgcgccaagctgaggtgatcaagggatcctggcagcccgtgggcgatctcctcattgactc
tctccaagatcacctcgagaaagtcaaggcacttcgaggagaaattgcgcctctgaaagaga
acgtgagccacgtcaatgaccttcctcgccagcttaccactttgggcattcagctctcaccg
tataacctcagcactctggaagacctgaacaccagatggaagcttctgcaggtggccgtcga
ggaccgagtcaggcagctgcatgaagcccacagggactttggtccagcatctcagcactttc
tttccacgtctgtccagggtccctgggagagagccatctcgccaaacaaagtgccctactat
atcaaccacgagactcaaacaacttgctgggaccatcccaaaatgacagagctctaccagtc
tttagctgacctgaataatgtcagattctcagcttataggactgccatgaaactccgaagac
tgcagaaggccctttgcttggatctcttgagcctgtcagctgcatgtgatgccttggaccag
cacaacctcaagcaaaatgaccagcccatggatatcctgcagattattaattgtttgaccac
tatttatgaccgcctggagcaagagcacaacaatttggtcaacgtccctctctgcgtggata
tgtgtctgaactggctgctgaatgtttatgatacgggacgaacagggaggatccgtgtcctg
tcttttaaaactggcatcatttccctgtgtaaagcacatttggaagacaagtacagatacct
tttcaagcaagtggcaagttcaacaggattttgtgaccagcgcaggctgggcctccttctgc
atgattctatccaaattccaagacagttcggtgaagttgcatcctttgggggcagtaacatt
gagccaagtgtccggagctgcttccaatttgctaataataagccagagatcgaagcggccct
cttcctagactcgatgagactggaaccccagtccatggtgtggctgcccgtcctgcacagag
tggctgctgcagaaactgccaagcatcaggccaaatgtaacatctgcaaagagtgtccaatc
attggattcaggtacaggagtctaaagcactttaattatgacatctgccaaagctgcttttt
ttctggtcgagttgcaaaaggccataaaatgcactatcccatggtggaatattgcactccga
ctacatcaggagaagatgttcgagactttgccaaggtactaaaaaacaaatttcgaaccaaa
aggtattttgcgaagcatccccgaatgggctacctgccagtgcagactgtcttagaggggga
caacatggaaactcccgttactctgatcaacttctggccagtagattctgcgcctgcctcgt
cccctcagctttcacacgatgatactcattcacgcattgaacattatgctagcaggctagca
gaaatggaaaacagcaatggatcttatctaaatgatagcatctctcctaatgagagcataga
tgatgaacatttgttaatccagcattactgccaaagtttgaaccaggactcccccctgagcc
agcctcgtagtcctgcccagatcttgatttccttagagagtgaggaaagaggggagctagag
agaatcctagcagatcttgaggaagaaaacaggaatctgcaagcagaatatgaccgtctaaa
gcagcagcacgaacataaaggcctgtccccactgccgtcccctcctgaaatgatgcccacct
ctccccagagtccccgggatgctgagctcattgctgaggccaagctactgcgtcaacacaaa
ggccgcctggaagccaggatgcaaatcctggaagaccacaataaacagctggagtcacagtt
acacaggctaaggcagctgctggagcaaccccaggcagaggccaaagtgaatggcacaacgg
tgtcctctccttctacctctctacagaggtccgacagcagtcagcctatgctgctccgagtg
gttggcagtcaaacttcggactccatgggtgaggaagatcttctcagtcctccccaggacac
aagcacagggttagaggaggtgatggagcaactcaacaactccttccctagttcaagaggaa
gaaatacccctggaaagccaatgagagaggacacaatg [symbol] tcagc
ctcga

[symbol]

[symbol]

[symbol]

[symbol]

[symbol]

[symbol]

[symbol]

gtaagttttttaacaagcatgggacacacaaagcaagatgcatgacaagtttcaataaa
aacttaagttcatatatccccctcacatttataaaaataatgtgaaataattgtaaatgata
acaattgtgctgagattttcagtccataatgttaccttttaataaatgaatgtaattccatt
gaatagaagaaatac [symbol] CGGCCGGAAGACAATAGCAG
GCATGCTGGGGAGAGATCTAGGAACCCCTAGTGATGGAGTTGGCCACTCCCTCTCTGCGCGC
TCGCTCGCTCACTGAGGCCGCCCGGGCAAAGCCCGGGCGTCGGGCGACCTTTGGTCGCCCGG
CCTCAGTGAGCGAGCGAGCGCGCAGAGAGGGAGTGGCCAA
SEQ ID NO: 61
SV40 NLS
Pro-Lys-Lys-Lys-Arg-Lys-Val
SEQ ID NO: 62
DNA sequence of the gRNA constant region
gtttaagagctatgctggaaacagcatagcaagtttaaataaggctagtccgttatcaactt
gaaaaagtggcaccgagteggtgc
SEQ ID NO: 63
RNA sequence of the gRNA constant region
guuuaagagcuaugcuggaaacagcauagcaaguuuaaauaagguaguccguuaucaacuu
gaaaaaguggcaccgagucggugc

| | Spacer Sequence | | |
|---|---|---|---|
| | (Sequence the gRNA targets | | |
| | and binds) | PAM | gRNA |
| gScb | GCACTACCAGAGCTAACTCA | NNGRRT | GCACUACCAGAGCUAACUCA |
| | (SEQ ID NO: 87) | (SEQ ID | (SEQ ID NO: 88) |
| | | NO: 9) | |
| g1 | CTTTACTTTGTATTATGTAAA | AGGAAT | CUUUACUUUGUAUUAUGUAAA |
| | (SEQ ID NO: 29) | (SEQ ID | (SEQ ID NO: 64) |
| | | NO: 89) | |
| g2 | TTTGAAATATTTTTGATATCT | AAGAAT | UUUGAAAUAUUUUUGAUAUCU |
| | (SEQ ID NO: 30) | (SEQ ID | (SEQ ID NO: 65) |
| | | NO: 90) | |
| g3 | TTTAAGTAATCCGAGGTACTC | CGGAAT | UUUAAGUAAUCCGAGGUACUC |
| | (SEQ ID NO: 31) | (SEQ ID | (SEQ ID NO: 66) |
| | | NO: 91) | |
| g4 | TTTAAATACATTGTCGTAATT | CAGAAT | UUUAAAUACAUUGUCGUAAUU |
| | (SEQ ID NO: 32) | (SEQ ID | (SEQ ID NO: 67) |
| | | NO: 92) | |
| g5 | TACCTTAATTTTGACGTCACA | CAGAAT | UACCUUAAUUUUGACGUCACA |
| | (SEQ ID NO: 33) | (SEQ ID | (SEQ ID NO: 68) |
| | | NO: 92) | |
| g6 | ATTTGACAGGTGAGAAATCTC | AGGGGT | AUUUGACAGGUGAGAAAUCUC |
| | (SEQ ID NO: 34) | (SEQ ID | (SEQ ID NO: 69) |
| | | NO: 93) | |
| g7 | TCATTTATAATACAGGGGAAT | AGGAAT | UCAUUUAUAAUACAGGGGAAU |
| | (SEQ ID NO: 35) | (SEQ ID | (SEQ ID NO: 70) |
| | | NO: 89) | |
| g8 | TTAAAGTCATTTATAATACAG | GGGAAT | UUAAAGUCAUUUAUAAUACAG |
| | (SEQ ID NO: 36) | (SEQ ID | (SEQ ID NO: 71) |
| | | NO: 94) | |
| g9 | AAATAGACACTGAAGAAAGGG | AAGAAT | AAAUAGACACUGAAGAAAGGG |
| | (SEQ ID NO: 37) | (SEQ ID | (SEQ ID NO: 72) |
| | | NO: 90) | |
| g10 | CCCCAATTAAAATAAAATTTA | CTGAGT | CCCCAAUUAAAAUAAAAUUUA |
| | (SEQ ID NO: 38) | (SEQ ID | (SEQ ID NO: 73) |
| | | NO: 95) | |
| g11 | TAAGTAATCCGAGGTACTO | CGGAAT | UAAGUAAUCCGAGGUACUC |
| (g3) | (SEQ ID NO: 39) | (SEQ ID | (SEQ ID NO: 74) |
| | | NO: 91) | |
| g12 | TTAAGTAATCCGAGGTACTC | CGGAAT | UUAAGUAAUCCGAGGUACUC |
| (g3) | (SEQ ID NO: 40) | (SEQ ID | (SEQ ID NO: 75) |
| | | NO: 91) | |
| g13 | GTTTAAGTAATCCGAGGTACT | CGGAAT | GUUUAAGUAAUCCGAGGUAC |
| (g3) | C | (SEQ ID | UC |
| | (SEQ ID NO: 41) | NO: 91) | (SEQ ID NO: 76) |
| g14 | GGTTTAAGTAATCCGAGGTAC | CGGAAT | GGUUUAAGUAAUCCGAGGUA |
| (g3) | TC | (SEQ ID | CUC |
| | (SEQ ID NO: 42) | NO: 91) | (SEQ ID NO: 77) |
| g15 | TTGACAGGTGAGAAATCTC | AGGGGT | UUGACAGGUGAGAAAUCUC |
| (g6) | (SEQ ID NO: 43) | (SEQ ID | (SEQ ID NO: 78) |
| | | NO: 93) | |
| g16 | TTTGACAGGTGAGAAATCTC | AGGGGT | UUUGACAGGUGAGAAAUCUC |
| (g6) | (SEQ ID NO: 44) | (SEQ ID | (SEQ ID NO: 79) |
| | | NO: 93) | |
| g17 | CATTTGACAGGTGAGAAATCT | AGGGGT | CAUUUGACAGGUGAGAAAUCU |
| (g6) | C | (SEQ ID | C |
| | (SEQ ID NO: 45) | NO: 93) | (SEQ ID NO: 80) |
| g18 | TCATTTGACAGGTGAGAAATC | AGGGGT | UCAUUUGACAGGUGAGAAAUC |
| (g6) | TC | (SEQ ID | UC |
| | (SEQ ID NO: 46) | NO: 93) | (SEQ ID NO: 81) |
| g19 | ATTTATAATACAGGGGAAT | AGGAAT | AUUUAUAAUACAGGGGAAU |
| (g7) | (SEQ ID NO: 47) | (SEQ ID | (SEQ ID NO: 82) |
| | | NO: 89) | |
| g20 | CATTTATAATACAGGGGAAT | AGGAAT | CAUUUAUAAUACAGGGGAAU |
| (g7) | (SEQ ID NO: 48) | (SEQ ID | (SEQ ID NO: 83) |
| | | NO: 89) | |
| g21 | GTCATTTATAATACAGGGGAA | AGGAAT | GUCAUUUAUAAUACAGGGGAA |
| (g7) | T | (SEQ ID | U |
| | (SEQ ID NO: 49) | NO: 89) | (SEQ ID NO: 84) |
| g22 | AGTCATTTATAATACAGGGGA | AGGAAT | AGUCAUUUAUAAUACAGGGGA |
| (g7) | AT | (SEQ ID | AU |
| | (SEQ ID NO: 50) | NO: 89) | (SEQ ID NO: 85) |
| g23 | GCACTACCAGAGOTAACTCA | | GCACUACCAGAGCUAACUCA |
| | (SEQ ID NO: 51) | | (SEQ ID NO: 86) |

SEQ ID NO: 96
gRNA targeting human intron 51
CTCTGATAACCCAGCTGTGTGTT
SEQ ID NO: 97
gRNA targeting human intron 52
CTAGACCATTTOCCACCAGTTCT

| Description | Forward Primer (5′-3′) |
|---|---|
| Surveyor | Fwd: CTGATGCTCTCCAAACTTGCC (SEQ ID NO: 98) |
| (g1, g5, g6) | Rev: TGCTTTGTGTGTCCCATGCT (SEQ ID NO: 99) |
| Surveyor | Fwd: ATACCTCTGAGATTGTGGTCCT (SEQ ID NO: 100) |
| (g2, g3, g4) | Rev: TGGGCAGCGGTAATGAGTTC (SEQ ID NO: 101) |
| Surveyor | Fwd: TTACTGAGTTTTAGAACCAGAGCTA (SEQ ID NO: 102) |
| (g7, g8) | Rev: AGGGTTCTTCAGCGTTGTGT (SEQ ID NO: 103) |
| Surveyor | Fwd: AGCAGGAGTCAAAGTACAGAGT (SEQ ID NO: 104) |
| (g9, g10) | Rev: TCCGGAGTACCTCGGATTAC (SEQ ID NO: 105) |
| gDNA integration | Fwd: TTACTGAGTTTTAGAACCAGAGCTA (SEQ ID NO: 106) |
| PCR | Rev: GAGTTCTTCCAACTGGGGAC (SEQ ID NO: 107) |
| CDNA integration | Fwd: CTGACCACTATTGGAGCCTCTC (SEQ ID NO: 108) |
| PCR | Rev: GAGTTCTTCCAACTGGGGAC (SEQ ID NO: 109) |
| 3′ RACE GSP | GTAGTCGTTTAAACCGCTGATCAGCCTCG (SEQ ID NO: 110) |
| ddPCR - Corrected | Fwd: GCTTTCTCTGCTTGATCAAG (SEQ ID NO: 111) |
| (Ex51-Ex52 junction) | Rev: GAGTTCTTCCAACTGGGGAC (SEQ ID NO: 112) |
| | Probe: GCAACAATGCAGGATTTG (SEQ ID NO: 113) |
| ddPCR - Unedited | Fwd: GCTTTCTCTGCTTGATCAAG (SEQ ID NO: 114) |
| (Ex51-Ex53 junction) | Rev: CGGTTCTGAAGGTGTTCTTGTA (SEQ ID NO: 115) |
| | Probe: AGCAGAAGTTGAAAG (SEQ ID NO: 116) |
| ddPCR | Fwd: GATGAGCTGGACCTCAAGCT (SEQ ID NO: 117) |
| Normalization | Rev: GTGGCTCACGTTCTCTTTCA (SEQ ID NO: 118) |
| (Ex59-Ex60 junction) | Probe: CGAGAAAGTCAAGGCACT (SEQ ID NO: 119) |
| Tn5-Top | CAAGCAGAAGACGGCATACGAGATNNNNNNNNNNGTCTCGTGGG |
| | CTCGGAGATGTGTATAAGAGACAG (SEQ ID NO: 120) |
| Tn5-Bottom | [Phos]CTGTCTCTTATACACATCT (SEQ ID NO: 121) |
| Tn5-GSP (g7) | AAGCAGTGGTATCAACGCAGAGTACCAGAGAAAATAGACACTGA |
| | AGAAAGGG (SEQ ID NO: 122) |
| Tn5-GSP (g12) | AAGCAGTGGTATCAACGCAGAGTACCOTTAATTTTGACGTCACAC |
| | AGAATG (SEQ ID NO: 123) |
| Tn5-Universal | CAAGCAGAAGACGGCATACGAGAT (SEQ ID NO: 124) |
| Tn5-BC, [i5 barcode] | AATGATACGGCGACCACCGAGATCTACAC[NNNNNNJCGGAAGCA |
| | GTGGTATCAACGCAGAGTAC (SEQ ID NO: 125) |
| Tn5-Read1 | CGGAAGCAGTGGTATCAACGCAGAGTAC (SEQ ID NO: 126) |
| (Miseq/Novaseq) | |
| Tn5-Index1 | GTACTCTGCGTTGATACCACTGCTTCCG (SEQ ID NO: 127) |
| (Novaseq) | |

SEQ ID NO: 128
Intron 51 of the human dystrophin gene
GTATGAGAAAAAATGATAAAAGTTGGCAGAAGTTTTTCTTTAAAATGAAGATTTTCCACCAATCACTT
TACTCTCCTAGACCATTTCCCACCAGTTCTTAGGCAACTGTTTCTCTCTCAGCAAACACATTACTCTC
ACTATTCAGCCTAAGTATAATCAAGGATATAAATTAATGCAAATAACAAAAGTAGCCATACATTAAAA
AGGAAATATACAAAAAAAAAAAAAAAAAAAGCAGAAACCTTACAAGAATAGTTGTCCTCAGTTAAATT
TACTAAACAACCTGGTATTTTAAAAATCTATTTTATACCAAATAAGTCACTCAACTGAGCTATTTACA
TTTAAACTGTTTGTTTTGGCACTACGCAGCCCAACATATTGCAGAATCAAATATAATAGTCTGGGAAT
TGATTATTATCCACTCTTCTAAGTTGTCTGTGCCAATTTGCCTTCTCCAATGATAAGGATAATTGAAA
GAGAGCTATAACTTAAAAAGAGAAAAGTAACAAAACATAAGATATTTAAAATTACCCTAGATCTTAAA
GTTGGCATTTATGCAATGCCATGTTCAAATGAACATGTTTTTAATACAAATAGTGCATTTTTCAGCCT
CAGTGTAATCCATTTGGTAAAATTATGACATCAACTAGAAACATTAGAATACATTGATGTAAATATGG
TTTACCTAGCTAGATCAAATATACTATATATCTTTTATATTTGTGAATGATTAAGAAAAATAATGTTG
GAATTGTTATACATTAAAGTTTTTTCACTTGTAACAGCTTTCAAGCCTTTCTAAAGAAATACAAAGTT
GTGCTGAAGGTATTTAGGTATTAAAGTACTACCTTTTGAAAAAACAAGAAGTGAGGCAGACAGAGTAA
GGGGAATTTCTTTGTAAAATAAACTTCACCAATTCCATAGGAATAAAAGTAATTTGATAGTAAACAAC
CTGCATTTAAAGGCCTTGAGCTTGAATACAGAAGACCTGAATTCAGTGCCATTTGCAAATGATGATTG
TGGTCAAGCCATCTCTGGATCTTCGTTTCCTATTCTGAGTACAGAGCATACAGAGTACACATTCACAT
TCACAATATAGTTATGGATATGGATGTATATAAATATATGTAAATACTACATATATGTACCTAAAATT
TGTTTTACTTCTGCTTTAAAAAAAGTAATTATAGCCACATTTTTCAGAAAAAGTAACTGAGGCTCATA
GATGTCAAATTCCCAGTAAGTAGCAGAACAAGGATTCAAATCCAAGTCCATTTGATTCCTAAGCTTGT
GTTATTACTTGCTACTGCAGAGAGTATACGTAGCAAGTAATATATGTACTGCAAGCAATACATACTAT
TGCTGCGGTAATAACTGTAACTGCAGTTACTATTTAGTGATTTGTATGTAGATGTAGATGTAGTCTAT
GTCAGACACTATGCTGAGCATTTTATGGTTGCTATGTACTGATACATACAGAAACAAGAGGTACGTTC
TTTTACAATACCATATTGAGTTATATAATACTCCCAGGACTTTTATTTACCAAAGGAAACAATATTTT
ATAATGTTTAAAGCCCAGGTTTTGAAGTTACATTGTCTGGGTTCAAAGCTTGGCTCCCAAGCTGTGTG
ACCTTGAGTAAGTTATTCTGCCTACCTGAGCCCAAGTTTATCTAGCTATAAAATGGGGATAGTTGTAC
TATCTGCCTTGCAGTTTGTCATCAGGATTAAGTTGGTTGGTACATGAAAAATGCTTCCCACTTTGCCT
TGCTTACTGCTTACTGCTAGTATTGAACAAATGTTAGTAATTATATTTGGTTCCACCACGAACTCTAG
AAATCTAACCAATGATGGCATTTGTATTATGCAAACTGTATATCACATCATAATATTATATGGAAATG
AGAGCTTGTTTCCGCTTCTGTAGCCTAGTCTACCATTGACATAGCTTCCTGCAGAAGTTACCAGATAA
TAGATTGGGAGAGAAAGTCCACACTTCCTTGTGACGGGTTTGTGAGTCCAGCATTTAGGGAAGCCATT
GATGTGCTCAGTAGTCTCCAGAGTTCTCTAAATAAATGTGTCCTTTTCAGAAAGGACTACTGATTTGA
TGCCCCCTCACAGAGATCGTCTTTAAATATAGGTCAAAAACTAATGTAGAGGGCCAGGTGCAATGTTT
CACGCCTGCACTCCCAGCGCTTTGGGAGGCTGAGGCAGGTGGATCACTTGAGGTCAGGAGTTTGAGAC
CAGCCTGGTCAACATGGCCAAACCCCATCTCTACTGAAAATAAAAAAATTAGCTGGTGTGGTGGCCCA
TGCCTATAATCCCAGCTACTAGGGAGGCTGAGGCAGGAGAATCACTTGAATCCTGGACCAGAGGTTCC
ATTGAGCTGAGATCACACCATTGCACTCCAGACTGAGTGACAGAGTGAGACTCCATCTCAGAAAAAAA
AAAATTTAGGGGGAAAAATCAAAAGCCATTTCTGAGACACAAAAATACAGGATTTATAAATTATATAT
GGTATATATAAAAATATTTTTAAAATAGTATATATAGCATATTATATATAATGATATGTAATGTTCAT
ATATTACATATTTATAAAAAAATCTAATCTCCCTTCTCTTGCTTGCTGAATAGGGGGATGCTTTGCCT
GCCTCTTCCTCTTATATTAAAAAATAATTCTTAAAGACATTGTCAGTTCTTGGCTTTTATAGCCTCAA
TCACCAAATTGTCGGTAAAATGGCCCTAAATAATCATTAAACAAATGTGTGTGAGAGGGGAAATAAGA
AGGATAAGTAAGTATGGGGAGGATTTTGTTATAATTTCAGGAAATCAATATCAATTTTATGTAAAGTT
TTAAATAAAGCAATCCCAACTTTAATGTTTGATGTGTGAAAAATTAGGCAAAATTCCAAAAGGGCTTT
ATAAACTGAAAAAAACTTTACTAACACCTATCCATTTTTATTATTTTAACCAACTTCTATTGAGCTGC
CACTAAGTACCTGGGAAACATAAAGTTGTACAACATAGAATGTGCAGGTAAAAGAGGTTGAAGGAAGA
AAATAATAACACTATGATAGAGATAAATTTTAGGATAATAGCTAACACATATGATATGCCAGTCATTG
ATCTAAGTACTTCACGTGAATTCTTTAATGCTTACAACATTACTGTGAGGTAGATAGAGAGGCACAGT
AAGGATAATAACCTGCCTGAGATCGAGGAAGAAAGACAATGATGAGATGTGAACTCAGGCAGTTTGGT
TCCAGAGTCCTCTCCCTTAAACCTCATAGTTTTCAACTTCTCTGATATTGTGTGGGTGATGCTGTTGG
GGCTTTCTTCAGGGAAAACTAAGCCAGGAGAGAGAATGGATGCTAGTGAGATATTCCTGAAGAAGGAA
AAACTTAAGCCAGGCATTAAAGAATGAGTTGGAATTACCTAGCTAGATAAAACGAGAAGGGCAATCCA
GGCAGAGGGAACAGACTGTGCTTTTCACTGAGGTGGAAAAAAAACAGAGTATATCAGAGGAATTGTGA
TTCCATATGGCTGAAGTTAAGGGTATATGATGAGGAAGAAATTGATGAGGTTGAATAGAGAGGACTGG
GGCTAAATAATGGGAATCCTTTGTTGCCAGACTGAGGAATTTTGATGATGGCCTACAGGCAGTGGCAA
CTCTGAAAGGATTGTAAACAGGAAAATAAAATCATCACATATAGTTTAGTTGCCTATCAATTAGAGCT
CTCTGGATGCAAGCAACAGAAATCATTCTCTGATTAAATCAGGCAGAAAGTAAATGTGCTGTAATTAG
CACAAAGGCATTGGAACAAAACTTACAAAAGGAAAAAGAATCTGAGCATGCCTTTCTGGGCATGTGGC
TAGCAAGAAGTATTCCAGTCTGTTTGTGATACTCTCTTTTCTCCATCCTGTGTGTAACTCTGTTCAAA
TTTTAAAGTCTTAAAAGAGAGTCCAGTTCACCTTGTTTGGGTCACATGTTAATACATGAGCTAGAAGG
GAGCAGAAAACTTTGATTTAAATCCCTCTCCTCCCAAAGTCTCAAAATTAGGGAAAGGCAATTCTCCT
GAATAGAAACTGGGTTCTATTGACAATAGAAGAAGGAAATGATTCTGACCAACCACTAAACAATAATT
GTCCACTGAACTCAGTCAAGAACATGTAGAATAAGTTGGAGGATAGAGCAAATAAAGGAGATTTGTAG
GAGGTAATTATTATGATCTAAAGCAAGCTTGTTCAACTCATGGCCTGTGAGCCACATGCGTCCCAGGA
TGGCTTTGAATGTGGCCCAACACAAATTTGTAAACTTTCTTAAAACATGAGATACTTTTTGTGACTTT
TTTTTGCTCATCAGCTATCATTAGTGTTAGTGTATTTTATGTGTGGCCCAGGACAATTCTTCTTCCAG
TGTGGCCCAGGGAAGCCAAAAGATTGGATACAGCTGATCTAAAGCAACAGGTTCATCTACTCAACTTC
ACAACGTGTAGACCTGAAATAAAGACCATTCATATACCAATACCYGAAATATAAATTTGTTTGACCAT
GACACGTACAGTAATTGGTTCTCAATAAATGTGGATAGCTTGATGGATAATGTGAATGCAATGTGATA
AGGAAACTTCATATTCAACAAAGACTGGAATGTGAGGATTATAATTCCAAAGCACCAGAAGATAGATA
AGATAATGCAATGAGACATTTTATGACTCAAGGCAAAGTTAGTTATGAGATTCAGACCAAACCTTAGA
CGTGCAGTAATTGAAATATTTGCCACAGAAGGGGTATAAGGACATGACATTCAAGTAAGCTAACCTTT
CACTAGCTTTAGACTTTGAACTCAGAAAACATATTTGGTGAAAAGCTTATGGTCCCCTTTAGTATGTA
TTGCTTGATTAAAGTATTATTTTAGAAAATGGTGAGCTGCTTCCATTTTGAAATAAAAATAATTTTTA
CTAAGTGAATTATATTCAGTGAAAAAAATGGAAGCTACAATTACAACTTTAATTTTTTTAAGTTTTAA
GAATACAGCCATTTAAAAAAATTAAGCAAATCTGCTTCATTTTAGACAGTAGAAAATATACCATTATC
TTTTAGAAGAATAGAGATGTGAAATATGCAAATTAAGCCTTTAGAAGTAAAGCACACATGAAGTTCAA
AGTTTAATTTCTAGAATTGTGAATCAATAGCAGTGGATGATTTGTACTTTATAGCTTAGTGTCGGAGA
AATCTGATTAAAAAATGCTTTTTCTGTTTCATCACATAAACATAAGTAAAATTGCTCTGAAACAACAA
TATTTGACAAGAATTAGCAGTTTTCTTTTTTGACATAATCTATCAAATGAAGGGAAAAATATGTCCTG
GGTTTTGCTTTGAGAGTGATTACTAAATCTGACCCTTAAGGAAAGGAAGGAGAGAACAAAGAAGGGAG
GAAAGAAAGGGAAGGAAGGAGGAGGAAAGGGAAAAAAAGAAGGAAGAGAGGAAGGAAAGCAGGAAAAA
GGGGAAGGAGAGAGAAAAGACAAAAGAAAGGTAGCAAGGAAAGAAAAAAAGACAAGAAAGGAATATTA
AAGAGGACAAAAGAGGAGTGAGGAAAGGAGGAAATGGAAGAGGGATGGTGGGAGACAGGAGGGAGAAA
GGTGGAGGGGGAAATATGAAGAGAGGTTCCCAGCAGTGGAGACTAGTGTTGCTATCAACAAATAGAAT
TTAGATGGCCATATGATATTATTTTTCATAATACTGGTGTCTGATTGCCTGTGCTGAGTTAATTGTAG
TCTTTTTTTTCAATTCCGTTTGGCCAGGTGTTCAGGATAATTCACCACAAAATCTCAACCACTGCACT
TGTATTGAATAAAGAATTGAGTTGGCAAAGGCATTTTATCCTCCAGTAAGACCTTTCCAGATTGGGGT
TGAGACAAATTGGCCAATCTGGACAAGATGATAATAGCATTGTTCAAGATTAATTTTTAACCACACAT
TGCACTGTTACCTGGGAGATTTCATTATCTAAAAATTGAATGAGCAGTTTTAGTGGGTATAGTGTATA
TTTAAATGGGACATAATTACTTGAATGAGTTTAATTTTTGTTGTTGTTGTTAAGGTCAAAGTACTTAA
AAATTATGATTTTTTAAAACTCTGTCTATACACAAAAAGCATTTGAATTAGCTACAGAATAATTCTGA
TTATAACTTTTGGTGAATAGATTCAGTCAAAATCTGATTACTAAACAACTTGTGTAGTATAGCCCTGG
AAGAATTGATGGGACAATGTGTGGGTAAAGTGGCATTGGCTATTTAAACTAAAAGCAATACAAAACAG
AATGTTTCTTGGTTTTATTCTGTTGTCACAAACCCAGCAGAAAGTGGCTATTACAATAGTTTCCCTTA
TTCAACAAATGAGAGAAGTTATAGACAATTTAGTTAATTGATCTAAAGTCACTTAGTAAATGTAATTG
TCCTAACATAAACCCAGACCCCCAGACCTCTTGGGAATAGATAATGTTTCTTACTTCTTTTCTATTTC
CTCAGCCACCCCCCTCAACTTCTTACACATCTCATTTCTCCATCCAAATTATAACAAAACAAAGCAAA
CATGGTTTATTTCCATGGGCATCAAATGGATTTCACGAGGTTGGGTGACAGTCATCTTAGGGTGAGGA
GATTGATTATTCTGTTTTTCTCTTTCATCGATCAACAATCCAGCCCTTCTCATCTCATCATTTCATTT
CTGCACAAACTTGTTTAAGAAATACCAATTAAGAAATTAATTAAGAAATTAATGTTGTAATCTGTTTG
GCTGAAGATATTTACAAATTTTGTGCTTTAATTATCTTCCAACAAATGTACATGTCTCTGGTAGACAG
CTTGCGACCATCTGGATGACTGATCCATATTTATATAATTTTCTTTCTTTACCTAATGAGACCAAATC
CACTATTATCTTCAACGAAGGATGTAAAGATATGTCAGTGTCAGTAATGTGACTTATTTTATATTCTC
TGGTCATAACAAAAATAAACCGCCCCTTAAATAAAAAGGTCATAGAGTTGCAAACACACACACACACA
CACACACACACACAAAATCATATTTTCTAAGTCTCCTAATTACCTTTTTATGGAAAATGATACCATAT
GCTTTTTTCTTAAAGAAACTACATAAACTTATAAACTATACTAAACTACACATTTCAAAGTCTATGAA
TGGAAATGTGTATCTTATTATATTTTAATTCAATTCACTGTAAACTTTTCTGTCAAAATCTTATCAAG
CAAAACTGATCCAGGATATTTACATGAATTCTGATGGAAGTCACTGTACTGTGTTTTCCATAAAATAC
CAGTGGGATTCTGATAAGGAAGTTTATGTTTGCCATTGTGTTTAAATAGAGAATTCTGGGCCGGGCAT
GGTAGCTCACGCCTGTAATCCCAGCACTTTGGGAGGCTGAGGCGGGTGTATCACCTGAGGTCAGGAGT
TTGAGACCAGCCTGACCAACATGGAGAAACCCCGTCTCTACTAAAAATACAAAATTAGCCGGGCGTGG
TGGCGCATGCCTGTAGCCCCAGCTACTCCTGAGGCTGAGGCAGGAGAATCGCTTGAACCCGGGAGGCA
GATGTTGCAGTGAGCCGAGATCACACCATTGCACTCCAGCCTGGGCAACAAGAGCGAAACTCCGTCTC
AAATAAATAAATAATTAGAGAATTCTATTTACAAATTTCCTTTCTTGGATCTAGTTAGGGCTCCTTTA
TATGGAGTGATTTTTATTGTTTTCATAGAAATACGTAGAATCTGGGTCTTCTCTAACTTTCTTACAGG
AAAGCAATGTAATAAGGTTTTTTTTTAATTTTCTGAAAGTTATATAATGTTATTGTTCCCTAAAGTTT
AGGACCTGCCTTTTAGGCTTTCCATTTCACCATAACTTTTGGTCCTTAAAGTCTGTAATTGAAGTTAC
AGTGTGTTATGATGTAAATTTTTCTTATTATTACCTTTAATGTTAGGGTAATGTTAACTAATGTTAAT
GTTAGGTATATGGTTGTTTTTTTCATTCCTTCGTTCAACAAATTGTCTTTGAAACCCATGTTACAAAG
CACTCTAGAGTCAGGTTGAAGACTATTAAGAAAGGAGAATAGAAAGAGACACTAGAGTAATAATTTGG
ATTTAAATTTGATTTCCTTGTGTATGATAGTGAATAAGTGTGAATAAGATGAGGCAGTGATCACACAT
CACTATTAGTAAAAGTGTTTCTGTACCTGTATCCACACTTTTATGTATATGGTTACTTATGTTAAAGT
GATACATATTATATAAAATTAACGTATACATTAAGTAGATATTTTAATAGTCTGTAATTAAATACTAC
TAGTATTTTCTTTCCTCCTTCAAGTGCTTACTTTTGATACCTCGAGTTACAGTGTCATAAAGATTCTT
TAGAAATATATTGACTGTCTTTTAAGAGCTTTTGATACAATACTGAGTTTACATTCATCTGTTATTTA
TTGAACACTTGCTGGTGAAAGGCATCAGTGTTATCTGCTCTTAGGGAACAAAAATTAAAAAGGGATAG
GCCCTAATTTTAGAGTGTATCCTCTATAAGAAAAACATAAAAGATAGGGCAGTCATGGCCACAAAAGA
AAAAAGTGTTATGGTGGTTTCAATCATATATGTATTAGAATGAATAAATCAACTGATCAATTGTGATT
TCTTATTCTAAATATGTGCCTGCCTTTTTCATATAGATGAAAATTAAGCTATGTTTATCTTTCCAGGG
ATCTTGTTGATTTTTATTCAATAACTTGGGAGTGAAAGTTGATTTTTGCATATGTTTTAATGTTTTTA
AATTTCATAAATGAATTGATCAGTAATTTCCAAGGTAGTAATGGCTGCATTGTTTTTGAAAAAAAAAA
AGCAACAGGATTTGA7TGTGCTTTTATGATTTTTAAAGAATTCATTAAAAATAATGCCACGGTTTCTA
AAATGATTTGAGTCAATTTCTTATTCGATTTATAAAAATAACTTTGAATACAATTTTAGTAATTCACA
AATGCTTTCAGTTCCCTTACCTTTATATTTTATATTCTGTGTAAACAAGTGACATAATATTTAAGAAT
TATATATCTCCTATGATTTATTCAAGAAAAGAATATATACTGTATTATTTATTTCAAGAACAGAAATG
CTTTGATTTAACTGTCATCTTCTCTCTTCAATTATGGAAGCAAAATAAACTGTAATGACCAATGTAAC
CCCTCCCCCATATCAAGTTAATCTATGTTCAACTCCAGAATTATTTTTGAACACTCAAACTAGAAATT
AAAAAAAATTAAATCCATGAAGACGATTTTTGCCAAAAGCATATAGATAAATTGAGTTGATTCTATAC
TTAAGAAAGTGGAGAGGAGAGAGTAATTTGGAGAGAGTAATTTACTCTTAATCCCATATTTTTTCCCT
AAATGTGAAAGAAGTAGATTGTAGTGAGAGGGAAAATAACCTGTAGCAACTTCATTGAGGCTAAGCTT
TCTGTCATGTTATATTATACGAAAGTAATGAAATGCTTCCACAGATAGAATCAGAAGTCCCCTCTGAG
AAATTCTACATAAAAATTAGCCTGCCACTTTACCACACTTACTCAAGTTTGATTTTTTTAAGTTATGT
AATAGATGTTAGGCACTAGAAGAGGACATTTACTGGGGGCAAAGATCAGTAGTTGGAAAGAATGCAAG
CAGGCAAGAAGCTATATATAATGAGATTTTACAGTACAATTGTTTTCTAAATGAAAATGAGGACGGGT
CCAGACACAATGGCTCACACTTGTAATATCAGTGCCAGGATGGAGGATCCCTTGAGGCCAGAAGTTCA
AGAGCAACCTGGGCAACAGAGTGAGACTTCATCTCTACAAAAAAATGAAATAAAAAGTTAGCTGGCTG
TGATGGTGTGGGCTTGTAGCCTTAGCTACTCAGGAGGCTGAGGTGACATGATCTCTTGGGCCCAGGAG
TTCGAGGCTGCAGTGAGCTATGATAGCGCCACTGGATTCCAACCTGGGCAATGGAACAAAACTCCATT
TCTAAAAAAGAATAAAATATAAAACTAAAATAATAAATAAATAAAAATGAGGATATATTTTATTTTAA
CATTTGGAAACTTTGTAGGTGAGGACCATGCAAACATTCAAGGTGTGAGTTCTGACCAAATCCAATTA
TTAACCATACCAATGACTTAAGGTTTCTTCACACTCCTTAAAGTTGATTAATATAATGATTATATAGT
TGACTGGTATGTCACAGCTTGAAGCCTTTGAGATTTATTCCTGCCTTTTCTGTAAAGGTTGTTTTGTT
AATTCCAGTATGTACTGGTCGTTTTTGTTTTGTTTTGTTTTTGTTTTTGTTTTGTTTTTTTGAGATGG
AGTCTCGCACTGTTGCCCGGGCTGGAGTGCAGTGGCACGATCTCGGCTCACTGCAACCTCCGCCACCT
AGGTTCAAGCGATTCTCCTGCTTCAGCCTCCTGAGTAGCTGGGATTACAGGCACTCACCACCACACCC
GGCTAATTTTTTTTATTTTTAGTAGAGATGGGGTTTCACTATGGTGGCCAGGCTGGTCTCAAATACCT
GACCTCATGATCCACCTGCCTTGGCCTCCCAAAGTGCGGGGATTACAGGCGTGAGCCACCGTGCCCGG
CTGCCAATATGTATTGGTCTTTTTCATCAATGATTCAGTCCAAAATCATTTTGTCCTTTAACTATATA
TTTTCTTGTAAAGCTGCTTCTGTTGTCTTGAACTTTTCTTTTCAAATGTATGTTGTCATTTGACTTTT
TAGATTGTTATTTTCTGGTCCTCGAAATAAATTTAAATTTCCTGTAAAGGAAGGTGTAATATTCTATT
TGACATAGCCGCTAAAGATGTACTAGGTGCTTTATAAATATTGTTGATTTACTTTATCTTCACAGATT
ACTAGTTTTACTTAGTATTTGGAATATGACAACATTTTATAGAGCTATATTCATATATATGTTTATCT
TAACTGTTAAATGCAATATGATTCATGTCTTGTTTTGGTCAATGATGAATGAAAGTCTCCTGAGAATT
AAATTTACTGCATCGATGCAAAAACAATCATAATTTTAGACACTCTAAGAATTTTAGAAATTAAAGGA
TTTTTTTTTTCCAGTTTACTCTGTTAAGATTGTGTTTAGCTATGCGTGACAGCATTCTCACTACAGTG
GCTTATCCAGATAGTTTCTTTTTCTCATAGAGCAAGACTTCCAGAATTATGTGTTCCAGGGTCAGTGC
AGCACCTCCAAAACCGTATGTCCCAACTTTTTCCTCCAACCCCAGTCATCTCCAACATGAGACTTTCT
TTTTGTTTTGTTTTGTTGTTTTTGTTTTTGTTTTTGTTTTGAGATGGAGTCTCTGTCGCCAGGCTGGA
GTGCAGTGGCGCGATCTCGACTCACTGCAACCTCTGACTCCCTGGTTCAAGGGATTCTCCTGCCTCAG
CCTCCTGAGTAGCTGGGATTACGGGAACGCACCACCACGCCCAGCTAATTTTTGTATTTTTAGTAGAG
ACGGGGTTTCACCATGTTGGCCAGGATGGTCTCGATCTCCTGACCTCATGATCTGCCTGCCTCAGCCT
CCCAAAGTGGTGGGATTACAGGCGTGAGCCACCGTGCCCAGCAAGACTTTTTTTCCTGTGGTCTCAAC
ATGGCTACTCTGCCTCCAGGCACTATGTCTGTACTTTAAAATGGAAGAAGGGAAAATGGGGAAAGTAA
AAGCATATTCCAGCTGTGTCAGCTCCTGTTTGTAAGGAAAACCAGTGCTTTTCTGGCAGCCCCACACA
GAAGAGTTTCTACTTGAACAGTGCATTAACCAGAAATGTGTCACGTGACCATTCCTAACTTCTAAGGA
TCTTGGGAGGATTGAGTGTTTTAACTGAATAGGTGTGTTTCTTTCTTCATAATTCAAAATGTGAAAAT
TGGTAACTTAGTTATAAAACCTTGCTAGTCTGAACAGAATTTGGATTTTTTTAGCTAAGAAGGAAAGA
AAGGGTATCGGATAGGCAGCTGGCTATGCTAGCCAAGATACTCTTAATAATGCACATTTTTCTTCTTT
GGACATAAGCAGTTTTAACTTAGCTAAATATGATGTGATTGTTTTCCTGTCTTCTTAGTTCTGTTTAA
ATTTGTTTCAGAAATCAAGGAAATAAAATGGAGAAAAAACTCTATTATTCATGTCTATCTTTCTGCCT
CTGAATATTTTTATGTTGGAGAAAGAGAAAGCAGTAACTTTCATAATAGCTTACATAGTCTGACAAAT
TCTAAACATGTCCGTTAGCATCAATATACGTGGTATTAGGTCATCAGTTTTTATATTCTGAATTATTA
AGACCCAAATAAACCCACTGAGTTCAAGAGAAAGTATACACTGAGCAATAAAAACATTACCAGTTCTA
GCAATGATAATCAAACAAGAAAACAAGTAATATAGTCTGTTTAGAATAACATGTTTTAAAGATCAAGT
TTTTCTTCCTTACCAATGTTGCCTTTCTTGTAACACTTTTTTTTCCTTCTTGAGATAGGCTTTCCTAT
CTTTTGTCACAAACCCCAATATTTACATGGCCATTCGTAGTCTATTCATAGCAGCACCACCCCATGGC
CCAAACTTGTAGATATTGCCCTCCTTCTATGGTTGTTCTAATAAGAATAACCACTCTTGTCTCTCATA
ATCTCAGCTGTTTTGTGCCGTTAAAATGGAAAATAATGAGTATTAAGATACTAACTAGGGGCCAGGCG
CGGTGGCTCACGCCTGTAATCCCAGCACTTTGGGAGGATGAGGCGGGCGGATCACGAGGTCAGAAGAT
TGAGACCATCCTGGCTAACACGGTGAAACCACGTCTCTACTAAAAATACAAAAAATTAGCCAGGCGTG
GTGGTGGGTGCCTGTGGTCCCAGCTACTCGGGAGGCTACTGCACTCCAGCCTGGGCGACAGAGCAAGA
CTCCATCTCAAAAAAAAAAAAAAAAAAAATACTAACTAGGTTTCAGTCATATGAGATGAATAAGTCCT
ACAAATCTGTACAGCCTAGGGCTTGTAGTTAACAATGTTTTATATTTAAAACTTTGCTAAGAGAGTAA
ATCTTTTTTTACCTGTTATTATCGTACATGCAAAAATAATAGTAAATAAGGAAGGTAAGAGAAAAATT
TTGGAGGTGATGCATAGGTTTATGGCATAGATTCTGGACATGATTTCACAGGGGTATACAGTCATGCA
TTGCTCAACAACAGATATACATTCGGAGAAATGCATGGTTAGCTGATTGATCTTGTTGTGCAAACATC
ATACAGTGTACTTACACAAACCTAGATGGTACATCCTACTATACATCTAGGTTGTATGGTATAGCCTA
TTGCTCCTAGGCTATAAACCAGTACAGCATGTGACTGTACTGAATACTTCAGGCAATTGGAACACGAT
GGCAAGTATTTGTGTATCTAATCACATCTAAACATAGAAAAGTTACAGAAAAATATTATAATCTTATA
GGACCACTGTCCTACATGTATACAGACTGTGCACATGATAGTGGTCCATTGATCAAAATGTCCTTATG
CAGCACATAACTGTAATTATCTCGAAACTCATCAAGCTGTGTTCATTAAATGTGTATAGCTTTTTATG
TCAATAAAGTGGTTAAGAAATCAATAAAGTGGTTAAAAAATATTTTGACTAGGAAATATACTATCATT
TCTAGTTGATAAAAGATCTCAACATTTCCAAAATTGTCCTACAGAAAACCAGGTTCATCAGGTGTTCA
TACATGATCCTCATGAAAAGGTCAAATAAGCTGAAAAACATGCATAGACGTTGCCTATCCTAGCAATC
TATGATGTACATCTCCATAGTAAGGTCACTGAAAAGTCTTTTAGGAATGTTAGTATTGTTAGCTCAGT
ATTTCTCAGTGATTTCTCCATGGAAACCATTTGTGTAGAGCATCTTGAGGAGCACAGCTGAGAGAACA
TTATCTTAGTTGGATGTGTATGTCCCTCTGAGTCACTTGATTTCTCTACATATGCTTTTACCAAATTA
ATCTTTTAGAAATCTT7TCTCTTCGCACTATGTCTATAATTTGTGAAGTTGTTACCAGGATAACATTT
GTGCCTCTCACCATGATGTACCTACCAGGGTCCAAGCAGCCATTCCTTCTCTAGAGCAACTGTCTGAG
GGAAAGAATTTAACACAGCATTCTACGAAATCATTTTATTTATAAAAATAGATTACTGCTTTACATAT
AGTAATTTATATTTAGAATATTGATTAATTATTAAAATCTGCATGAGAGCTTTAAAGAGTAGTACATA
ATATATAGCAGTTTGTACTCAAACTGTCTTCTAAAAAGGATTCACTTTTTGTTTGTATTCTATTGTCC
TATTCGTTGATAGTGTTACGTAAGTAATTATAAAACTTAAAATCTGGAAAGAGAATGTGGACTCAGAA
TGCCATCTCTTTTGTTATTTCAAATGGATTAGAAATGAACATACATATTCATTTTCTTTCATTACACA
TCCAGAGAAATAGAATGGATTTTATAAATATGTAAAAGCAAGGATTTTGATCACTGATAAAAAGGGAA
GGTTTGGTCACTACCTTATTTCATTCCTTTTTTCTTATCCTTTTTTTTTTTTTTGTCAATTATTTGAT
GACATCTCTGAACATCACCTTTTATTCATGACAAGAATTGGGTATCATGGTAAAGAACACTGTTAATA
TAATTCAGTTACTTCACCCCCTCCTGAAATATAGAGAAGCTTTAAGACTATGTGAATATTTTTTTCTG
GTTTTCTTGTATTTGTAGAAATAGCATGAGCTTTGTTTAAAGTCAGGCATCTAAAACCTTGCCCTGTA
TGTTATTGACAACCTGCACAAATTTTAGGATCTATTCTATTACAGTTTGTTCAACTGTAAAACTAGGA
TAGCAAACTCTATGTCATATTTTCGTTATCAGAATTTAAAAAGCATGTTTTAAGATCTTAGTAAATAA
TAAATCTCTACTCTGTAGTTGAATTTGTTCTATATTCTTTAAGAAATTCCCTTTGATGGTTATGCCAA
CCTCTGTATTACTTTTCTTCACACTTTAACTTTGCGCTGAAATCATAGTAGTATTTTACGTTATCAGT
CAAAATAACAGTCATCCTTAAAACAAATATGAATTTTAGATGATTAAATAGATTTGTATGGAGGTTCT
TCTTGCTAATCATAGCAGTTATCCTTGGTGAAAAATGATAGACACTTGAAAAAACCAATTAATCATGA
TGGCTATTTTTGCATCATAAATAAAGCTTTCAAATTTGAGAGGGAATCAAAAGGGCAATGGTAGTATA
GTGTCTCAAAGCCCCTTTCCAATTGATGGTACAAATTTAAAAAGAGAGAGAGAGAGAGAAACATGTTT
CACTGTAATTGTTTTCTAAGAGCTTCCAAAAAAGCGTATTTTCTTAATAGATTCAAATTTTTCAGTTG
GATTGAAAGGGAAGTCTTGGAGTGTAGTGAGGAGGGCACCTTCTGTTGAGAGGTGTTCAGACGACAGA
GTGTGCCCAAGGCCAAAGATGAGATGGTTTTGCGAAAGTCAGTGGCCACAAACAGGTGTGTTTGACCC
CTGAGAGATATGCAGGAAGTCTACCCCACTTTAATTCTTCCAAATATTCTTTACCTTAATTCCCAAGT
ACTTGATAAAGGAGCAATGGGGAGAAAATATGCACACTATTATGGAAAAGTTTTGACCTACACTTTGG
AGAGTTTTAGATTAAGAGCATTCTAGAAATCAGTCCCAAATGCCTAGGGTTTACTTACTTAAAGATAA
TATCATAGTTTGGGTGACTGGGAAGCATACCCTGAGATTGAGGTGAGCATGCAGTATGTCTATTTAGG
AGTGTTCTTGGGGTCAACGTGTAGGGGCAGAGGGAGAAGTTGAGCTCTGACGCAGTCTTAGTAAGGGC
CTCAGCTGACCGTTCAGGGAGTTCTTAAGCTGGAATGACCCTTCAGAAGTGCTAGGAAACGAAGAAAG
GGGACTGGATCTTTATAACCCCGTGTCAAGTCATGCACTGGATGTGGGCTACTCCAGGAAGGCAACGA
ACTTTAGCAAGATGATTCTCTTTAGCCACGGGAATTTCCATAAGGGGGCTGCTATGGTCTGAATGTTT
TTGTCCCTCCAAAATGTGTATGTTGAAACCTAACACTCAAGGTGATGGTATTAGAAGGTGGGGGTTTG
GGGGGGTGATTAGGTCATGCGGGCTCTGCCTTCAGAAACAGGATCAGTGCCCTTATAAAAGCGGCTCC
AGAAAGCTTCCTTGCCCTCCCACCATGTAAGGACACACCGAAGATGCCATTTAACAGGAGTGGGCCCT
CACCAGACAATGAATCTGCTGATGTCTTGATCTTGGAATTCCCAGCCTCCAGAACTATAAGCAATAAA
TTCTGTTGTTTATAAATTACCCAGTCTAAGGTATTTAGCTATAGCGGCCCAGACTAAGACAAGGGCTG
ACAGCTGAAGGCTGTCTACCAGCAGCACTCCTAGCAGCTGGGGAACTAAGTCCTTCATTTCCAAAGGG
GAATCTAGGCAGCATATTTACAGCTTTTCACTACAGATAAGCTCATTATTTCAAATAGGGACTAGCAG
GAAAAAATTAAATTGCCCAAAATTTAGTGGGATGCTGAAATAGATTGTGGTGTGTAAATTGGAGTATA
GTGAGGAGAGCACCTTCAAACCAGTATGTACTACATGATATTGTTTTTGTTGCAATATTTATTATATA
CCCAAACACACATATATTACTTTTAGAAACACACACCACATATATATCTATGAATATTTTATATACAC
ATAGGGAAGGATTGTTGATGTTATTTATGCTATTTTAAAGATCGATGTTTTCATATAATTATGTATTG
GTTATATATTATTTCTTGATATAAGGTAAAAAAAAAAAGCAAAACAAACTTTAAGTGATCACTATGAA
AAGAATCCCAATGCTGCACATTTAGGTTTATCCAACTCTTCCCATTAAAATATTAAATAGTAGAAATA
ATTGTGAATAAGAAAGAGCAGATTTTGAAAAATGGAAAGAAATGCTTAAAGACATAGCATTGTTGCCC
AACCATCATTATTTAAACATACAGTGTTTGGCTTTGACCAAATTGCCTTCAAACACTTCCTTTTGGCC
CAAAATGTTAGGTCATATATACTACCATAAAATTCATGATGCTTACCATGCATTAATTTCTAGTATAT
ACCAGGCATTGTGCTATGCATATCATATTCAATATTTCTAATCCTCTCAAAAGTGGTACAAGCTAACT
GGCGTTTTTCTTGTTTTGAAAGGGAGAAACTCAGAGAGGTTAAGTGACTTGCCCAAGGCAATGCCATT
GATAAGTGCCAGATTCTATCACAGGTTTATTGGCAACAAACCATATGTGCGCGTGCATGCGCGTGTGT
GTGTGTGTGTGTGTGTGTGTACACATACACGAATAACATATATGGTATAAATACGTGGAAACATAATA
AACTGCATTGAGCTGCGTTTATAATTAGTATTTAGGACATGTTTGGCAAATAAAAACAGTGGAGATTG
AAATGGATTTGCTTAGGAAAAATGATACATTAAAATAGGCTTTATTATGAGTCTTCAACTATTCTGTG
AAAATAGATACCCAGGGAAGAAATAATAGAGAATATGAATCTTGAGCAGGCAACTGAGAACTTGTCGA
AGAGCCAAGATAAAAATGTCAGAGAGGAGAATATTTTGGCAGCTCAGATGAGCCCCCAGAGGGTGGGA
GGCAATGATCTCACCGCAGTCTCGTATCTGAACCCCAGGTTTTTGCATCTCCATAAAGTAATTTCTTA
CACCCCTCAATAATGATCGGGCTTACTCTCAATCTCTCGCTCTCTCTCTGTGTCTCTCTCACGCACAC
AAACATGCAGAACATTTCTTGCACATGCATAACTCATAAGACGATTATGTAAATACCAGCCTTTTTAT
TTCATAACTAAATTACAAGGCCTGGTTATTGTTTGGACTGTGAAAAAATAATTATGTGAATAGGTGCC
TCAAGATGAAAGACAAGGCAAGATTGTGAAATTATTCATATGATAGTAATAGTATGCAAAAAATAACA
CAATCTTTAAAGATCTTTAACGACCTAGTTATAAAACCATGCTTTATAACAAATATAACCATGAGGAA
ATAAAAAGAAAAATGTAATAATATACTCCAAGAATAAAGTCAAATGTATTGTTGAATGTAAGGAGTTG
GTTACACTTCCTTATAGTGGAGGTTATTTTAAAATTTGTGGCTTACGTGGTGTTATGAATTGCCCTAG
ATCAACACTATTATGCAAGGCCAACTATTAGGTTATTTTTGGTAGATAACCACAGCAAAACTTTAGTA
TAATAGGTAAAGGTTAGCTACACTCCCATACCCTCACTCTCAGGTGTTGTCATACTCCGTATAAAAGG
TTCAATCAAGGGAGACATGAGAATATTCCAGAATCTAGAGGCAGGATGCAGTTAACCTTAGAGAAGGC
ATCAGACAACTAGAATCTTCGGATTCAATGTGGAAACAAAGCATAGTTTAGGCATTAAATCTTGGGCA
CCATTCCAAAGAATACAGGTTCCATAACTTACTATATTTTTATACCTAGCAAGCTAGAGATGAGGAAT
TGCTCTCAAATATTTTAACCAAAGCATGTATCTTAAGTAACACTAATCTCATAAGTGAAAACTCATTT
CTAATATTCATTTTGCTCATTAGCAAGGCCTCTAGTGTTGACTGTGATAAAAAATAGTTCAAATGCTG
GTAGAACCCACCCCAGGAGACTGGCCTTTCTGATTAAATTCTAACTCTATCCCCACGTGAATTCCTGA
CTTAAGTAACTGAGTTCCTGCACATCAGAATATAAGTATATTATAGATATAAAAACATATGTAATTAA
TAAATATTTTAAGTGAGACACTTCTTTCATCTTTATGGCTTAACTATATCAGACATTTGATTATTTTT
AGCGGTCTAACTACAAAACAAAACACAAAGCCCACAACTAAAAATTTCTTTGTATATATTGCAAAGAG
GCAACCATTTGGTGTCAATTCAATCATGAGTGAAATGCTATTATACGAGTACATCTCCCTGGCTTGTA
TGGGGGTAATAGGGCATGGAATTTACAGATTCACAATAACTGAGATATTCACAATAACAAAGATATCA
ATATGTAGCTTTTCCCATAACTTTGTGTAATGAAATCCTCAGTTTGTGCTGTGTAAAAAGCTTATTGT
TTACTTCTCATGAAAATCATCTTAGTTTTTATCTTTATTTAATAGTCTGTAATTTGGGGGTAATACAT
TCGTTTTGTTGATACTATGTGAAGTGGCAAGCAGAAAATTCTAACAGGAATAGATAAGCAAGTATCCT
ATAAATCAGAGTCAGTGTCTCTCTCTCTCTCTTTTAATGAGTCAGTCTGTCTCTCTCTCTTTTTCCCT
GCCTGGCTATCTATCTGTATTTTTCAGTTTTGCTTTGCAAATAAGAGAATTGTGTGTTGTAAACCAAC
CAACTTACCATTAATTTTCTCTGAATTCAAAAGCAATTACAAGCGGACTCTTGAGTTTGTGCTGCCTG
GTTGTCTGCATATAGGCCAGATGTCTAGAATAGGATCTTTATTTACTATTTTTACCCTCCTAATTTCA
TGGTAACTCCAAGGTAGATGATATTTGTAATCGTACACTACTTGTCAGAAATCTTTCTAATAACACTG
CTATTTTATAAAAATAAACATTAATTCAGTATAAAATTTTATTTTAAATTGTTAATTCAAGCAAATCA
GTGAGGTAACTTTTACACTGCCGAGCGTACGTGTGTGTGGATTAGTACAGCCATGCCATAGACTTCAC
TTGTAATCTTTTCTTTATATTTTTTATACACCTGAAATGTTCATCATTGTGCTGTAGAAAACAATCTC
ATTGTGTTTTTAAAAGCTAGAGTGGGTATTGAGAAGGGGAAGAGGATCATAGAAAAAGTTGGTTAACA
TGCTACTTAACACTTCAAATCTTTACTCGATGTCATCATCAGCAACATTTTAAATTTATGCTTCTACT
AGTTTGCAGTTCTTTTCCTTTGATTATTCTTATGATACAAGCCTTTCCACACAAAATTTATGTACAGG
AATTGTGTAGAATTTTTCTTTGGAAAATATGGTGATTTATTACAATTTGGGCAACATCATCATTTTAA
AAATTCAGAATTTGATTTTTCTCAGAATCATCAGAAAGAATAAAGCATATATATGGTTCATGTCAGGA
GAATTAGAACATGAGAATTAATATATCTCTGATCTTTTAAAATATTTTCATGTTTGTGAATCAGCAGA
TTTTTCCTAGTTTGAGATTTAAAAAATCTAGATATAATTAAAATCTCACTGATGTTTCACCATCAGAT
GATTTTATATTTGTATTTTCTTCCACTTCATAACTTGTATAGAGAAGAATAGAAGAAAGAAAAAGGGA
GGATTGATAATCTTTCTCTCTCAGTTCTTATAGCACTTCATTTTTTAAACTTATTACTTCCTTCTGCC
TGCTTTGTTTGTCTACATGTTTGTATTTCATGATTTCTTAGAAATCCATCTACTGCCATTCTGAAGGT
CATTTACCTGAAAATGATAGAAAGCAGCATATATTCAAACAACTGCAGAGTAATTGTCTATATCAGTT
ATCATTGTTCATTACTTTTCTGTTTTAGGATTGAGGGGCTGCCTCGCCACCTCCCTCACACCCCCAGC
ATATTATCACAAAGCCTACTGATTCATTCACATCCCTGGGCTGAATTTGCCACCCACTGTGTGTTCCT
GTTGTTTTGTGTATGGAAGTGAAAAGATTTAATTTGATGTTGTTGAAAAGACACAGAGGCTAACTTTC
AATTTTCATATGTAGTTCTTCCCTCTCCCTCTGCACCACCTCCTTTACTTGTTGAGAAAATTGCCCTC
TCCATGGTAACAATAGAAGAAGCTTTCAGATTTTAGTAGTAGTTGTTGCAGAGAAAAGAATTCAAAAA
GTAGATGAAGTTTAAAAATGAAAAAGAGAGAGGAAGACAGCTGGGAAGAAGGCTTAATGTTTATGAGT
GGGTGTGGAGGGGAAGAACTAAGTTGAATGAACAAAGCTGAGCTAAGGGGAAGATGGTTTTTCTGCAT
CCCAGAAGGCAATACCCTAGCCTTTCCTGCAGCCTTCACTCCCCAAAAGATAAGAGCTTTATCTGAAA
TTCTTATAGGATTCATTCCTGAAGAGCAGCTTGTCACCAAACAGAAACACTGTGATTTCCTCAGGGAG
TCACAGTTTATTATTATTTTTTTAATGTAACGCTTTTGTGAACTCCAGTTTCCACCTCAATTCAAATG
GTCTTTTGGTTACAGGGTGAAAGAGACCCAACAATACACCTTTCCCACTTCCGGAGGCCTTTGGTTAA
ACCATGTCTGCCACAAGGACACAGGAGCCTGGTATGACTGGTTGTTTTTTGTTTGCTTTTTTGCCTCC
TGTGCTTTCTAGATTGTGAGATACTGTAACTCTTGTCGATGACACATAGTACCGAACCCACCCGAAGA
AGTATGTCAGTATGTCACATTGTGACAAACAGCTTCTCATGCTAAGTAAATGCAGAACCATTGTGAAA
GGTTTAATAATGCCCACTCCTCCCCCGCCAAAGATGTCCATATCCTAATCCCAGGAACCTGTGAATAT
GTTACCTTACATGGCAAAAGGCTTTGTATTAACAGATGTGGTTAAGTTAAAAATCTTGACACGGAGAG
ATAGCCTGGGTTACCCCAGTGCGCCCAATGTAATCACAAGAGTCCTCCTAAGAGAGAAGGAGGTGATG
ATACAAGCAGAGTAAAAGAGAGATTGGAAGATGCTACACTACTGGCATTGAAGATGAAGGACAGGGCC
AAGAGCCAAGAAATGCAGGCAGGCTCTAAAAGCTGGAAAAGGCATGGAAAAGAATCCTCCCCTACATC
CCTTAGAGGGAATGCAAGCTCTGCCAACACATTGTTTCTAGCTTGTGAGACCCATTTTTTGGACTTTG
GACCTCCAAAATTGTAAGATAATAAATTTGGGTTGTTTTAAGCCATTAAGTCTGTAATCATTTGTTAC
AACAGCCACAGGCAGCTAATACAGCCATGAACATTTAGTAATGACTAACTTTGCACAATTTTAATACA
AGCTTCTTATTAAGGTTTATTTTTTCTTAATTACAAGGAATAAAAGTGGGGTCTGGGGGCAATGTCAT
GGTCCACTCCGTTTTAGCCATATGAATTTGTATTTCCAGCATTAGAACAAAAGGTGACAAATCTGAAT
GTATTTGTGTGAAATAATAATAAAGCAGAACAAAAAGGGAAAAGTGTCCAGCTGGAAATGAAGTTAGA
GAAAGATGAGGAGAAGCAAGCCAATTGTGTAGTTTTCCCTTCTGCTTTTTAAAATCATGATTTGTTTA
ACCCACTGAATTCTATTTTAGAAACAGGACTGCAAGGAAGTGTTGATGGATTTGGTGGCATGAGAACC
AGAGTCACAGAGGCAGGAAAGTAAGGAATAAGTGTTAGAATAGGAAGCAGAGTTGCTTGGGAAGAGAC
CTTATGACATGTGGACAGGGCTAGACTTAGGAGTCAGAAAGACCTGAGTTCAAATGCTATCCTTTAGT
ATAGTTTGAAGTCAGGTAGCGTGATGCCTCCAGCTTTGTTCTTTTGGCTTAGGATTGACTTGGCGATG
CGGGCTCTTTTTTGGTTCCATATGAACTTTAAAGTAGTTTTTTCCAATTCTGTGAAGAAAGTCATTGG
TAGCTTGATGGGGATGGCATTGAATCTGTAAATTACCTTGGGCAGTATGGCCATTTACACGATATTGA
TTCTTCCTACCCATGAGCACGGAATGTTCTTCCATTTGTTTGTGTCCTCTTTTATTTCCTTGAGCAGT
GGTTTGTAGTTCTCCTTGAAGAGGTCCTTCACATCGCTTGTAAGTTGGATTCCTAGGTATTTTATTCT
CTTTGAAGCAATTGTGAATGGGAGTTCACTCATGATTTGGCTCTCTGTTTGTCTGTCGTTGGTGTATA
AGAATGCTTGTGATTTTTGTACATTGATTTTGTATCCTGAGACTTTGCTGAAGTTGCTTATCAGCTTA
AGGAGATTTTGGGCTGAGACAATGGGGTTTTCTAGATATACAATCATGTCGTCTGCAAACAGGGACAA
TTTGACTTCCTCTTCTCCTAATTGAATACCCTTTATTTCCTTCTCCTGCCTGATTGCCCTGGCCAGAA
CTTCCAACACTATGTTGAATAGGAGTGGTGAGAGAGGGCATCCCTGTCTTGTGCCAGTTTTCAAAGGG
AATGCTTCTATAGTACAAGGCTACAGTAACCAAAACAGCATGGTACTGGTACCAAAACAGACATATAG
ATCAATGGAACAGAACAGAGCCCTCAGAAGTAACGCCGCATATCTACCACTATCTGATCTTTGACAAA
CCTGAGAAAAACAAGCAATGGGGAAAGGATTCCCTATTTAATAAATGGTGCTGGGAAAACTGGCTAGC
CATATGTAGAAAGCTGAAACTGGATCCCTTCCTTACACCTTATACAAAAATCAATTCAAGATGGATTA
AAGACTTAAACGTTAGACCTAAAACCATAAAAACCCTAGAAGAAAACCTAGGCATTACCATTCAGGAC
ATAGGCATGGGCAAGGACTTCATGTCTAAAACACCAAAAGCAAGGGCAACAAAAGCCAAAATTGACAA
ATGGGATCTAACTAAACTAAAGAGCTTCTGCACAGCAAAAGAAACTACCATCAGAGTGAACAGGCAAC
CTACAACATGGGAGAAAATTTTCGCAACCTGCTTATCTGACAAAGAGCTAATATCCAGAATCTACAAT
GAACTCCAACAAATTTACAAGAAAAAAACAAACAACCCCATCCAAAAGTGGGCGAAGGACATGAACAG
ACACTTCTCAAAAGAAGACATTTATGCAGCCAAAAGACACATGAAAAAATGCTCACCATCACTGGCCA
TCAGAGAAATGCAAATCAAAACCACAATGAGATACCATCTCACACCAGTTAGAATGGCAATCATTAAA
AAGTCAGGAAACAACAGGTGCTGGAGAGGATGTGGAGAAATAGGAACACTCTTACACTGTTGGTGGGA
CTGTAAACTAGTTCAACCATTGTGGAAGTCAGTGTGGCGATTCCTCAGGGATCTAGAACTAGAAATAC
CATTTGACCCAGCCATCCCATTACTGGGTATATACCCAAAGGACTATAAATCATGCTGCTATAAAGAC
ACATGCACATGTATGTTTATTGAGGCACTATTTACAATAGCAAAGACTTGGAACCAACCCAAATGTCC
AACAATGATAGACTGGATTAAGAAAATGTGGCACATATACACCATGGAATACTATGCAGCCATAAAAA
AGGATGAGTTCATGTCCTTTGTAGGGACATGGATGAAATTGGAAATCATCATTCTCAGTAAACTATTG
CAAGAACAAAAAACCAAACACCGCATATTCTCATTCATAGGTGGGAATTGAACAATGAGAACACATGG
ACACAGGAAGGGGAACATCACACTCTGGGGACTGTTGTGGGGTGGGGGGAGGGGGGAGGGATGGCATT
GGGAGATATACCTAATGCTAGATGACGGGTTAGTGGGTGCAGCGTGCCAGCATGGCACATGTATACAT
ATGTAACTAACCTGCACATTGTGCACATGTACCCTAAAACTTAAAGTATAATAATAATAATAATAATA
AAATCTCAAAATAATTAAAAAAAGAAACAAACAAATGCTATCCTGATCCTAACTGGCTGGCTGTCTTT
GGGGAAGTTGGTAATCTTTTCTGTGCTTATTTCCTCATGTGTAAAAAAATGAATATAGTACCCAGCTA
GGTAGAGTTGTTGTTGGGATTAAATGATGACTATAAAGCATCTAGCCCAGCTTCGGCTACATTATAGC
TGCTTACGAAATTGTAGTTACGATGTAAAAGAGAAAAACACTGGAAAAGGAGGATATGGGCCATTTTA
TTCCACCTTCACCACCTTTTAGCTTGGTGACCTTGGGCAAATTATGCTTCATTCCGTGCTTCATTTTC
CTTGTCTATAAAAGGGTGTAAGTACAGAACCATTGAGGGGTGGTCATTATTAACCTACCTCAAATGGT
GTCTGTAAGTTAATATATATTGTGCTTTTCCTATGTACAATATCTAGCACATAATTACAAATCAAATC
CATCCCATGTGCAATATCTAGCACATAGGAAAAGCACAATAACTAGTTATTACTCTTGTTGTAGTAAT
TGCTACGCTGTAGGAGTTTGAATTGTAAGGCAGTGGAGAGTCACTGACCTTTACGAGAAAGTGTAGCA
GAACATTTGAGTAGATAGTAATGGGGAATATTACATAAATGGATAGATATTAGGGGCAGATATTACTA
TTAAAATATTACAGCATGGATATTTATTAAGGCCAAACTGGTTAATTAGTTGCATCTCTCAGGTTCCT
AATGTTGCTTAATTTTTTAACCTCCCATTTTGTGCTGCCCTTTGTACGAATATTTAATGCTCCCAACA
CCTCTTCAGTAGCACATGTACTGTGAGTTTGTTTTGTTATTACTTGTGTGTATTAGCATTCCTTTGTG
AACCAAAAGCATGGAATTAGCTGTTGCCTCTAGGCTACCTAGTTTTGTAGTTTGGATTGAAGCCTTCA
CCTCAGTAACACCTATTCTGTCTACTATCTTACAGAAAACTTGTAAAATTAAGACAGATCATTAATAT
AGCACAAAGAGACAAAGGGCAGAGAACATTGAGATACTGGATATTGGAACCACCCAATAGTGTTGATT
TATTTATGATTATCAGTTTTTGTCTCTGCCTAGCCTCATGCCACTAAAGTCTCTGAGGCAACAAAGAA
TAAGCAATTTTGCTCACCTTATACAAATAAAACACAGAAAAAGGAATCACTAGAGAAATGGTACTGCA
GCCTTTCTGCAGGGATTACTGCTTATTTTTAAATTACTTAAAAGGTATTGAAATTATTGTTCATAATG
AGAAACCTGCCTAATAAAACAGAAAATTAAACTTAACACTTCCCTATAATGTAAACAGCTCGGTTAGG
AACACAACATTACAGAAACCACTTAAGAATTGATTGTACTTGTTCTTGGAGCAGAACTAGAAGCTCAC
CGTTTAGAAGCTGTGCACATTTCCCTATCAAACAGTACATAAAGTTTCCATATTCCTCAGAATCGGCT
TCATTTGTGCCATGTGTTTGCTTGGAACTATGCCACAGAAAGCAGTTCTCCCCCTCAAGCTGGGCTCC
TTTCATGCCGCAGTGCAAGTGTGTGATATACTGGCACCATGTGCTAATGTAGACCCATTTTTATATGA
TAAGAATTAGTACGGCCTAGGGAATAGACAAGTATGTCTAAAATCCTCCCCATAGAATATGTCCCTTC
CTTTAAAAGCTGTCATACTGTAAGTTCCAGCTGAGTTAAAGGCCACTGTGCTCCTATAGGGAAATATA
TTCTATTGTAATTTTTACGTTCTCCAATAACAGTCTGTTCTTTGTTTACTGAAGAGAGCTTTCATGTC
ATAAAATGGTGTTTTTTGACAGAGAAGCAGAATCATTGTTTTATTATAGAAATTTGCTCTTACAACAG
CAAAAATAAATAGCTCATCTCTTAAGCTCCTGATCAATGTCTAACACCTCCTACCCCCAGCAACACTT
CACTGCAAGTATATTAACACTCTATAATAGCAATTCCACTCACCTACCAAGAAATGATCTTCACAAAT
GATTTACAGCTAAACCAGAGCTTAAACACATAGCACCCAATCAAGGGCAGATTTTTATCTTTTTCCCA
GTCATATAAGTTCTGAGAAGAAATAGATTAATGTTGATCTCCCAGACAACTGCTGAGAAAATGTACAA
AGGATGTTGTTTATTTTGAAGAATGAGACCTAGTTGTTAAGCACTTTTTCCCCTTATATGTACGTCCA
AAGGTAACCATTACACCATTTTGATGCAAATTTAGGATATATATTTATTCATACCTCTCTTCTCCATT
CGGATGTTGTCTGTGTGAGTGCTCACAGACACATGCACACATACACACATGCACTCCTGTTTCACACT
TATTTGTAAAACTCACAAGGATTTCCAAGCCATTAATATAGCATTGTTTAAGGTGAACACATGGTTGT
TCACCATCCATATGTATCTTCACTTTGTAGCACTCAGAATTTGGCAAAATCAGAAGGCTGAAACCTCA
TGGATTAAATATATTCTATATAACATATGTCTTAATTGCTGTTACTGTAAAGAAACCTGGACTAGCCA
TATTTGACTAATTTCTACCTAAGGTATTTGAATTCTTATAAATAGATTCATTGCTTTAATCACACAAG
AGTGGTTTATATGAATGTAATTATCTCCACTTTATAGCTGAATAAACTGAGCCTGATTCTATCCCTAT
ATGGGAAACATGAATTGAACAGCTGTGCCAATTATTTTGATAATTCAAATTTCACATCTACCATGTGA
AGACAGCAGAAGAGGGTTAGGGGGCTTGAATTATTCTGATTAACTGTGTTCATGAGTGTAATCGCCTC
TAGATAATCACTCATTTCTTCACTTCACTTCCCATCTACAGGTAGTATCAGCGAATGGTAACATCTCT
TGCTTTGCCTCAGTTTATTGATGGCTGCCGTTAGAAATAAAAAGCATGGTTTTGTTTCAGTACTTAAA
TGAATAATATCTTCAAAATGTTTTTAAAACGTGAAAAGGTTGAATGCATTTTTAACAAATGTTTTGTT
AGCTTTGACTTTTATTTTTGAACAGATGAGCACAATACCCCAGTACTCCTTTCCTAGAAATAGGAGTA
CTACCTGAAGACTTATTTCCCAAAGAAAAATATCAGGTCTAGTGCAGCAATACGTATTAAGGGCATTG
AAAAGTTATATTCACAAAATGTGACATCATAACATATGTTAATACTTCTTATCACTGATAATAATCCT
TGAAGTTGTATTTCCAGAGAGATCTCAATTTCTTCTCACACTCTGAAAGTCTCTGTTTATCCTTTAGA
GTAGGAATGTAAGAATTTAACAAAACATTCTGAATGTTTACCTTTTTTCTAAACTGAAACTACAATCC
CTTTTTACCCCTATATAGTAAAATATAATATTACAAGTAGAATCAGCAAATTTGTTTAATAATTCTTT
GGGACAGTTTTTACAAGCAATGGGGTTGAATTTATGTTCCTTGTGTGCAGTGGAGTTATTATATTCTT
CTTAAAAAGATGCATGAAGGTAAATTAGAAATGTTTTACATGTTTTCATGACAGGACATTTAATCAAA
GAGGAGATACAAGAGGCTTTTCTTGGGTTACTGCAATTTAATTTTCCATTTCTTTCTTGGAAGGGACA
TTGGATGCAGTTGTACGAGGTTAATATTTCTAACATGCCACTTTTATTGTGGCATTCTTCTGCTCTCA
AACCTCTGATGATTTCCCATGGCCTTCAACATGATGTCCTTATTTGGGCATCTAGTGTTCTGAGCCCT
CACATTCTGGCCCCAGCTTCCCTTCTCAACTTGATCACAGCCATCGTAATTCTGCTAACTAATTACAC
AGCTGCCCTTCCCACTCTTTCTCAACCACTCTGCTGTATTCTGACTTCTACACTTTAGTTTTAAAGCT
ACTCCTTGTCCTGAAATTCCTTCTCCCATTAGGTCATTATTAATTGAGTTCATCCTCTAAGTTACAGT
TCATGTTGCTGTTCCTCTATGGCACCTTCCCTGAACCTGGTCCCATATAAAATCCCATCTCTCAGAAT
CCCATTAGGGTAGGATATGTGGTCTGTAGACTATTACGTTTGTCATTTACATATTGTCTTCTATTATT
GGGTAACTGCGTGTGCGAGCATGCATGGGCTGGCCTGAAGGTCACCTCCCCAACTGCATTGAAAGTTC
ATCACAAGTTCAATTATTTCACTAGAGAGTCTCTTTCATGTCATCCATGAGGCACATTCCCTACTGTG
ATGTGTTATGCATACAATTATTTCAATAAATATTTTCTAGCTCTTTGATTGACCAAAGCTTAATTACC
TGTCAACTCTAGCCTCTTGTATCTGGAATTTCTACAGTCTTTGGATAGTATCTTTAGGATGCAAAATT
AGGAGGAGTATGTACCAGGCAAACTATTACAAATAATGCCCTCAAATAGTTACATTTCACTATTCATG
TCTTGTAATTTATCTTCTGCTTTGGTATTTTAGTACACTTATGATTCAATTTGCTGTATAGATTCCTC
TGAATAGGGACAAGAGAATTCGTCTTGATAAGTGGAAGTTCGAAGGATTCCAAAATGATGTTATTCAA
GGTAGAACAAGAAATTAATACTGAAAAAATTGAGGAGTAATAATCCCCAAATATGTACATGCGTATCT
CGTTTTATTGGGTTTCACTTTATTGCACTTTGCAGATATTTCACTTTTTGTAAATTGAAAGTTTGTGG
CAAGGCTGCATTGAGCAAGTCCGTCGGGCACAATTTTTCCAACAGCATGTGCTCGCTTTACGTCTCTG
TGTCACGTTTTGGTAATTTGCTCAATATTTCAAACATTATTATTATTATTATATTTGT7ATGA7CTGT
GATCAGTGACCTTTGATGTTACTATTGTAATTGTTTTGAGATGTCATGAACTGCACTCATATGAGATG
GCAAACTTTGTGGGGTGCAGTGGCTCACACCTGTAATCCTAACACTTTGGGAGGCCAAGGCAGGAGGA
TCGTGTTAGCCCAGAAGTTTGAGACCAGTCTGGGAAACAAAGTGAGACCCTGTCTTTAAAATATATAT
GTAGAAAAATTAACTGGGCATGGTGGCACATGGCTGTAAGGAGCCCTGCCAGCTGCATGGGAGGCTGA
CACAGGAGGATCACTTGAGCCCAGGAGGTCAAGGCGGCAGTAAGCCATGTTCACTCCAGTGCCCTCCA
GCCAGAATGACAGAGCAAGACCCTGTGTGGAAAAAAAAAAAAAGACAAACATTTTTTCAACTTTGATT
TTAGATTCAGGGAGTACATGTGTAGGTTTATTACCTTGATATATTACATGATGCCGAGGTTTGGAGTA
CAAATGATACTGTCACCCAGGTACTGAGCATAGTAACCGATAGTTAGTTTTTCAACCCTTGTTTCCCT
AGCTCCACATGAGAACACGTGGTATTTGGTATTGTGTTTCTGCATTAATTCACTTAGAATAATGGCCT
CCAGCTGCATCCATGTTGCTGCAAAGGACATGATTTTGTTCTTTTTTATGGCTGCATAGTATTCCATG
GTGTATATGCACCGTATTTTCTTTCTCCAGTCTGCCACTGATGGGCACCTAGGCTGACTTCATACCTT
TGCTATTGTGAATAGTGCTGAAATGAGGATGAGAATACATGTGGTTTTTTAGTAAAGCAATTTGTTTT
ATTTGGGCTATATGCCCAGTAATGGGATCACTAGGTTGAACGATAGTTGTGTTTTAAGTCCTTTGAGA
AATCTTCAAACTGTTTCACTGTGGCTGAACTAATTTGCATTCCCAGCAACAGTGTATCAGAGTTCCCT
CTTCTCTACAGCGTCAGCAGCATCTGTCATTTTTTTGACTTTTTAATAATAGCCAGTATGAATGGTGT
GAGACGGTATCTCGTTGTGGTTTTGATTTGCATTTCTCTGATGTTGAGTGATGTGGAGCATTTTTTCA
TGTTTGTTGGCCACTTGTATGTCTTCTTTTGAGTAGTGTCTGTTTATGTCTTTTGCCCATTTTTTTTG
ATGGGGTTATTTGTTTTTGACTTGTTGAATTGTTTAAGTTCCCTATAGATTCTGAATATTAGACCTTT
GTCAGATGCATAGTTTGCAAATATATTCTCTTATTCTGTAGACTGTCTGTTTACTCTGTTGATAAATT
CTTTCACTGTGAAGAGCTCTTTAGTTTAATTAAGTTCCACTTGTCAATTTTTGGTTTTGTTATAATTG
CTTTTGAGGACTTAGTTATAAATTCTTTCCCAAGTCTGATGTCCAGAGTGGTGTTTCCTAGGCTTTCT
TATAGGATTCTTATAATTTGAGATCTAATGTTTAAACCTTTATTCCATCTTGAGTTAATTTTTGTATA
TGGTGTAAGGAGGGGGTCCAGTTGCATTCTTCTGCATATGGCTAACCAGCCATCCCAGCATCATGTCT
TAAATACAGAGTCCTTTTCCCATTACTTATTTTCATAAGATGGCAAACTTAATCAATCAATGTTTTGT
GTGTTCTGGCTACTCCACTGATCAGCCATTCCCTCATCTCTCTTCCTCTCCTTGGGCCTCCCTATTCC
CTGAGACACAACAATATTGAAATTATGCCAGTCAGTAACCCTACAATGTCCTCTAAGTGTTCATGGGA
AAAAAAAGAGTCACATGTTTGTCACTTTAAATCAAAAGTCAGAAATGATTAAGATTGGTGAGGAAGGC
ATGTCAAAAGCCAAGACAGGCTGAAAGCCAGACCTCTTGTGCCAGTTGGCCAAGTTGTGAATGCAAAG
GAAACGTTCTTGAAGAAAATTAAAAGTGCTACTCTGTTGAACACAGGAATAAGAAAGTGGAACGGCCT
TATTTTTAATATGGAGAATGTCTTAGTGGTCTGGATAGAGGATCAAAACAGCCACACCATTATCTTAA
GCAAAAGGCTAATCTAAAGCAAAGGACTAATTCTCTGCAATTCTGTGAATGCCGAGAGAGGTGAGGAA
GCTGCAGCAGAAAAGTTGGAAGCTAGCAGAAGTATGTTCATGAGCCTTAATGAATAAGCCCTCTCTAT
AACATAAAAGTGCAAGGCAGAGCAACAAGTGCTGATGGAGAAGCTGCAGCAAACTATCCAGAAGATCA
AACTAACATCTAAGTTAATAAAGGTGGCAATACTAAACAACACATTTTCAATATAGACGAAACAGCCT
TCTATTGGAAAAGGATGCCATCTAGGACTTTCATAGCTAGAGAGAAGTCAATGCCTGGCTTCAAAAGT
TCAAAGGACAGGCTGACTGTCTTGTGAGGGGCTAATGCAACTGGTGACTTTCAGTTGAAGCCAGTGCT
CGTTTACCATTCCAAAAATCCTAGGGCCCTTCAGGTTATGCTAGATCTAGTCTGCCTATGCTCTGTAA
ATCAAACAACAAAGACTAGATGACTGCACATCTCTTTACAGCATGGTTTGATGAACATTTTGAGCCCT
CTGTTGAGACCTACTTCTCAAAAAAATGTGTCTTTCAAAATATTACTGCTCTTTGACAATGTCCCTGG
TCACCCAAGAGCCCTGAGGAATATGTCCAAAGAGAATGATTTTATTTTCATACCTGCTAACACAACAT
CCATTCTGCAGTCCTTGGATCAAGAAGTCACTTCAACTTTCAAGTCTTATTACTTAAATCATACATTT
CATAAAGGTATAGCTGCTATATTGTGGTGAGTCCACTGATGGATCTGGATAAAGTAAACTGAAAACGG
AAAGTCCTCACCATTCCAGATGCCATGAAGAAAATTCATGATTGAGGGGAGGAGGTCAAAATATCAAC
ATTATCAGGAGTTTGGAAGTAGTTAATTCCAACACTCATCAATGACTTTGAGGGTCCAAGACTTCAGT
AGAAGAAGTCACTGCAGATGTGGTAGAAATAGCATGAGAACCAGAATGAGAAGTGGAGTCAGAAGGTG
TGACTGAATAGCTGTAATCTCAAGGTAAAACTTCAGCGGATCCAGAGTTGCTGCTTATGGATGAGCAA
AGATTGTGGTTTCATGAGATGCACTCTACTCTTGGTGATGATGCTGTGAACATTGTTGAAATGACAAC
AAAGGATTTAGAATATTCCATAAACTTAGTTGATAAATTAGCATCAGGGTTTGAGAAGATTGACTCAA
ATTTTGAAAGAAGTTCTACTGAGTAAAATGCTATCAAACAGCATCACATGCTACAAAGAAATCATTGG
CTATAAAGGAGAGCCAATCGATGCAGCAAACTTCGTTGTTGTCTTATTTTAAGAAATTGCTACAGCCA
TCCCGACCTTCAGCAACCGCCACCCTGATCAATCAGCAGCCATCCTCACTGAGGCAAGAGCCTGTACC
AGCAAAAAGATTATGACTCCCTGCAGGCTCAGATGATTGTTACCATTTTTTTTAGGAGTAATGTATTT
TCAAATTAAGGTATGTACATTTTTTAGATACAATGCTATTGCACACTTGACAGATGATAGTAGAGTGT
AAACATAACATTTAATGCACTGGGGAACCAAAAATATTCGTGTGCCTCATTTTATTGTGATATTTACT
TTATTGCAGCAGTCTGGAACCAAACACACAATATTCCAAGGCAGTCCTATATATTGTTTCATGTCTCT
CTTTCTTCTAAAATGTGTGTAGGAGAAAAAATTATATCACTTATGTTACTGGGCCATAATATCAAAAG
CCTGCGAATCTGATGCACATGATAAAAACTGGTCTTAAACCTGTCTTGATTATCCTTTGTTAATATGC
CAAATTTATAGAACAATAGAGTTTCAAGAAATGTGAACAATGTAGAATAACTAAAAGATCTAACGTTG
AATAGCTAAAATTATCAACGCCCTTTATATCACTTTAGAAATGCGTTTGCAAATCATTATCAACAAAT
TGTAGCATAAAATTCTTTTTTTTTCTTGACTTTGAAATTGTATTTCAAGATCCGCAATTTACACCACA
TTATTTACTTACTGGCTTGTGAAAGTGAAAGGCATTTTCATTTTTGGATGTTAAGGGTTTTAGTAAAT
GACAAGTAAATCAATCCTTTAAGTTCCGTGTGGTATAATAGCTTGGAAAGGACCATCTTAATCTTTTT
TTCAACACAGGGAGATAATTTATTTTAAAAATAACACACTAATAGTAGATTAATATTATTACCATTTC
AAAGAAGTCACCAAATTTGGTAGGCTTAAGAGGTTTTTTTTTTTTTTTTGAGATTACTATGCTCTTTT
TTTATTTTATTTATTTTATATTTATTTATTTATTTTTTAGTTTTTTAATTTTACTTTAAGTTCTGGGA
TACATGTGCAGAACGTGCAGGTTTGTTACCTAGGTATACATGTATCATGGTGGTTTGCTATACTCATC
AACCCAACATCCAGGTTTTAAGCCCCCAATGCATTAGGTATTTGTCCTAATGCTCTCTGTCCCCTTGC
CCCTCACCCCTTGACAGGCCCCGGTGTGTGATGTTCCCCTCCCTGTGTCCATGTGTTCTCATTGTTCA
ATTCCCACTTACGAGTGAGAACATGCAGTGCTAGGCTTAAGAGTTTTTTTAACTCCCCAAAATATCAA
CAAATTGAAACATTACTACAAAGAAATAGAGAAATAAATTTCACTGACTGTCTTATTGTTTTTTAAGT
TTGAATAGCTAATAATGATTTCTTAAACAGCTATCATATTTTTTATTTTTAAAGCTAGCCAAATGATC
AGTGATTTTTATAATACTGATAAATACTGCTTAGAAAAGGAACATGTGTTCTAGCAATTTCCACACAT
TTCTGATTCTAATTACTTGTTTCTTTTTGTTTTATTTTTATATTTTTAAGTTTTGTGAGTACCTAGCA
GGTGTATATATTTATTTGGTACTTGAGATGTTTTGATACAGGCATGCAATGTGTAGTAAGCACATCAT
GTAAAATGGGGTATTCAACCCCTCAAGCATTTATTCTTTATGTTCCAAACAATCCAATTATACTCTTT
TAATTAATGTAAAATGTACAATTAAATCATTATTGGTTATAGTCCCCCTGTTGTGCTATCAAATAGTA
GGTCTTACTCATTCTTTCTAACTAATTTTTTGTACCCAGTAACTATACCTACACCACCCCCACCTCCC
CACGACCCTTCCCATCCTCAGGTAACCATCCTTCTACTCTCTATGTCCATGAGTTCAATTGTTTTGAA
TTTTAAATCCCACAAATAAGTGAGAACATGCAATGTTTGTCTTTCTGTGTCTGATTTATTTCACCTAG
CATACTGACCTCCATTTCCAACAATGTTGTTGCAGATGACAGAATCTCATTCTTTTTTGTGGCTGAGT
AGTATTCCATTGTGCA7AGGYACCACATGTTCTTTGTCCATTCATCTACTGATGGACACTTAGGTTGC
TTCCAAATCTTGGCTATTGTGAATAGCGCTGCAATAAACATGGGAGTGCAGATATCTCTTCAATATAC
TGATTTATTTTCTTTTGGGTATATACCCAGCAGCGGGAATGCTATATTATATGGTAGCTCTATTTTTA
GTTTTTTGAGGAACCTCCAAACGGTTCTCCATAGTGGTTGTGCTAATTTACATTTCCACCAACAGTAT
ACAAGGGTTCCTTTTTCTTCACATACTTGTCAACATTGGTTATTGCCTGTCTTTTGGATATAAGCCAT
TTTAACGGGAGTGAGGTAATATGTCATTGTAATTTTGATTTTCATTTCTCTGGTTATCAATGATTCAG
TGATGTTGAGCACCTTTTCATATGCTTGTTTGCCATTTGTATGTATTCTTGATCTGCCACAAGTCTCT
AATTACTTGTTTCTTGTACCACTGTTTCCCTTCATTGTAGTCAGTGATTTGGTCAATCAACACTTTTT
ACGTATGGGTATCTGTTAACTGTATTTCTAGGAATGGACCAAGAATTGAGAAATTTATCCCAACCAGA
AAGAACAAAATCTATGGAGGCACAGGATTACTGAAAGCTTTGCTCCTATAGCCTCAGTTTTTTTCTGC
AAGTTCGCTGCTTCCCAGTTGTCCTTGTGATAAAATTCAAAACCTCAAACTGATTTTTAAAAAGCCAA
CTATATACTGCTTCTACTATGTCTACCTAAACTTATTTGCCATTATTTGTAAAGAATTCCAGGCTCTA
ACCAGCCCATTTGAGGCTTTATTTTACGTTCATGCCTTTGTTCATGTCTGTCTTTCCCTTATAATGCT
CTTCTCATTCTCTAGCCAAATCTGCCAAAGTTCAGCTGTAGTGCCATACTTGATATGAAGTCTTTGCT
GAGAATTCCCATATGTGGTCATTTCCTTCACTGATTTGTTTCAATAGTTGTTGCCATTATTATTATTT
TTTTTATACTGTTGCTGGATACTTTCGCAGATCCAATCTCTAACCTGTTGTTCTACCCTAGGAGGTTG
ACCAATATGGCAGTAGCATTGGGCTTGCTTGTCTATTGGCTTGGTCCTTGTGTTGGGAGGCATCAGCA
GATCAATGGATGAGAGGAGAGTGAGTCGAGGGATGGCTAGGTTCCTTTACTCACAGCCCCAGCTCCTG
TCAAGAGGTCTTGTCCCTGCAGCCACTTCTCAGATTCTACTAACTATACCCTCCCCTTAGCCTTTCAG
GCCTTGAGGAGGAAAGCCTCCTCATCCCACTTTGAATGAACTATTTATTGCAAGAACCATGACTGATT
CAGAATATAATCTGTCCTTGAATGAAATAGTTAATTTCCCCTTTTGTATTCTCGTTTCATATCCCTGT
CCCTTTATCCATTCCTATATTATATCATACTATCATATAGCTACCTTATATTATACTATGACATGAGT
ACCTTGTAAGTATATCATGCTATATTATAGTTCTTAAGTGTGTGGGCTTTGGGGCCAACTTACCTATG
TTAGAAGTTCTGCCACTTATTACCATGTGATCCTAGACAAATTGTTTAACTTCTTTTTCATTCATTTT
CTTTACTAGTAAAATAGGGGCAATATTAGTACCCACCTCACAGAGTAATATGATAATTACATTGACTG
ATAGTTTTTATTTAAAACTTTTATTGTTCTTACTATGAAATGGGAAATGTTCTAAACACCTTACAAAT
ATTAACTCTATTAATTCTCATAATGAATCTGAGAGGTAGAGACTCAGTATCTCCATTTCACAGTGCTG
GAAATGATACAGACAGCATTTAAGTAACATACTGAAAATAGTAAACCGAATAGGTGGCAGCCAGTATG
CAAATTGGGAAAGCCTGACTCCAGAGTTCATTCTTTTAACTAGTATGCTGTGATAGACTCTTTTGGCA
TATGGTATGCACTCAATAAACAACTATTGTTGTTGTCGTTAGCATTACTTCCCATTGTATTATTACAC
AAAAAATAATTGTTTATGAGTATTTGTTTGCTAGATTATAAGTTCCTTGAAAAAAGAAGACAAGTTTT
ATTTGTCTATGTGTACTAGCTGAGTGCTTGGCAAATAGTTGCAGTTTAGTAAATGTTTCTAAAACAAA
TTATTAGTTGTTTCTTATGTATTTCCCAAGTCTATCCTAGCCTTGGAAACAGCTAACACTTAGCTAAA
CCTAGAAATGTCATTTGAGATTTCAGCAGCCATCATTGTTGCTGAAGCCACAGGTTCTCCTTTTTATC
TCCAATTTCTTCCTCAGATGATTCACCACTTTCTTTGTCGTTTCCCTACTTCATTTTTCCTATAGTGA
CTTTTGTTCTCCAATAACCATTGATAGTGATGACAGTCCCACCTTGGAACCACTTCTAACTGTCCTGT
TCAGCTTTTCCTGAGGCCAGATTTCCCCCAAAATACATTTTTTAGCTTCATAACTGCTCCTTCCAGAA
ATTTGAAACGCATTCTCAGGAATTAAGCAAGAGGCATTTGATTGACGTCATTTAATTTGTGTTTAAAT
GTGTTCTTCCCATCAACGATTAACCAGTGCTTCAGCCAAGATACATAACTTTTATTCTCCACTAACCT
TAGGGTTGAGGTCACAATCAGGTACGTAATCTGTAGAGCCCAGTGAAAAATGAAAACGCAGGGCCCTT
GGTTAAAAAAAAATTAAGAATTTCAAGATGGCAACAGTAGAGCATGAAACCAAGTATGAATCCCTTCT
AATGCAGATCCTTGTGTAACTAACTGCACAAGTTATACGTTCAAGAAGCTGGTCCTGGTTGAGGTCTT
TGTCTACCTAGGGCACTTCTCACTCAGAGAGGTGGAATAATCTTAACTTTCTGTCTTCCGTCATCTGA
AAACTTGCCCCAAAGTTCCTTTTGCCATCTCTTGCAGCAAACCTAGTGGTTGTTATCTGTTAAGGATT
CCTGGTGGCTTGCTTTAGTTTCCTACAGTTCTTGAGCTGCATGTGTTGAATGGAACTCCACCATTACC
ATGTAACACATCTGAATACTCTTATTTTCCTCCAGTTAAATTCTGCGCTCCTTGGAAATAATGGTCAT
GCACCCTACCTTAGTGATTTCTAAATAGAGGTAGTATGTAAAAATACATATTTACTTGAATTGCATCT
TAAGAGGCCAGCCCATGGGAGGCATGAAGAAACCTATGTTACTAATTAATATTCAATAATTGACAGTT
ACACATTCAGGTAGTAGTGTCATGGAACCATCACGTTACATGGAACTGAAGAGCTGATGAGAGGTTTA
GGGTCTAGGAGGACAAAAGTGAGTGTATTAGTCAGGGCCTGAGGTTATATGTACCCAGACAGGATAAA
ATGGGACAATATTTTATTAGTGGAAATATCTATGTGAAAGAAAAGAAGGAGGAAGCCAAGGAAGGTGC
AAGAGATGTTAGATCAAGATGCAATTCTGACTCTGCAAGGAAGAGAGAGGGAGAGAAGGCTAGGCAGA
AGCATCCCAGTGTGCGGTCTAGTGGAAGGAAATTTTGGCAAAGCTGTTGGGAAGTCATTGAGGCAGAG
CCAGGCAAAGAAGTCCCATGTCTCCCAAGGAAGGCTCTCTGCCTTAGTATTCCCACCACACCCAATCA
TTGGGTGAGGAGAAGTCTGTGAGAAGCTTGGCTTTGGTGCAGTGCAATCATGGATTTCAAAATGCAGT
AACAGGAGTCCTCAGTCAGTTAAGACCCAATAATAGAAGGCCTGCATATTCTCATGGTGGCCACTTGG
GTATGAGGAGCAGTGGTGTTTAAAACTGTTTTGTATAATTTAAATAAAGAAAAGCTGAGGACTACTTA
AGCTTGATTCCTTCAGAAGACAGTCTTTGGCCTTTATATTTCATGTGATATCTTTGCCATATGCTCTG
ATAACTCTCCATGTTTCCCCTACCATAATACCTCAGGTTGTTGCAATTGCTTTTTTATTGCCTGTCAT
CACTACTCGTTTGTTTTCTCTTTGAGAACAGGAATTATTTTCTCCTTCACTGCTACAGCCCCTGCACC
TAGCTCAATGAGTGACACAACAGAAGCACTTAAAAAATTGCTATAACTCAAATTTGGGGGATATTCTA
CAAAATACCTGGCCTCTGTTGGTCAAAACTGTAAGTGTTATAAAGTTCAAAGAAAGGCAAATAACTAT
TTCAACTTAAAAAAGACTAAGGAGATACGACAACTAAAAGCAATGCGTGAGTCTGGACCAGACCAGGA
AATAAAAATATAGCTATAAAGTTCATTAATGGGGTAATTGTCATACTCTGAATATAGAGTATGGATAA
GATTATGGTACTAAAGCCACGCTAAATTTCCTGACTTTGAAACTGAGCTGAGGTTATTTAAGAGAATG
TCTTTGCTATTAAGAAATAAAACCTAAAGTATTTAAGGGTAAAGGGGCATGATATCTGCAAATTATTC
TCAAATGATTCAGAAAAAAAATATATATATATAAAACATTACATATACAGTTATATATCTATACACAC
AGAGAGAATGATAAAATGATAAATAATTAAAAATAATGTGGCAAAATGTTAACAAATGGTGAATCCGG
GCAAGGGTTACCTGGGAGTTCTTTATCTTGCAAATATTCTGCAAGCTTGAAATTATATAAAAATAAAA
TGCATCTTATAAAGTATTTATTCAGTGAATAAAGAACAAAAAAGGCTAACTGCAGTTGGAAGATATTT
ATGAAGTTGGTTATGAAGATTCTTAAGAAGTTACCGATGGAGGTGCTAGTAGAACATTCAAAAAAGAA
GTAGGGAAGGTTGACCAAGTAGGAAAATATCAATATCCAAGCCAGTATAAAATGGAAATGAGAAGTGA
GGAAGTAAGTGGAAGACAATGAGGATGGTTTTCGATTTGGCCACTCTATTTGGAGAGGCAGCCGAATG
CAAGAATAGAAGCCAGAAGAAGATGCTCAGTGAGATGGTTGAAAGCTAGATAGATTACAGCATCCTCA
CCAGTAAAACCCTTTCGGTAACTAGAAAGGCTACAATTTAGTACCTTCCTGACTTCTATGCTTATTTT
CTTCAATACATAAAATGGTTCCGTAAACTCTTTTACCTTCTGAATTCTTTATATTAATTTTTTGAAGT
TGTAAATAAAATAGCATCAGTTCTACATTGTTACATTTCAGCTTAATTCATATTCATTTACTGAAAAT
GGGAACATTTGAAAAATCATCATGGGCATTTATGCTATGTAGATTGTTGATTTTTATAGAAAAATATA
AAAATATGACCAGTTTGATTTTCAAAGTCTTTTCTTAGACATGTAAATACTAAGCATTCAACTCAACA
TATAGAGTTTTTATTTGAGTATTATTTAGGTGGAATTCTATTTTAATGAATACAATAAAAAATTGTAA
TTTTGTCTAAAAGCCTAAAATGCCCTAGTTATAATATGTATGATTTCACTGTTTAACTTCCTATTTCA
TAGGGTTGCTATTTATAACCACTTCACTCAACTCTGGGGGGACTTAGTGAGATTAAAGACTTCTGATT
CACTTTGTATTTGAAGAATTTTTTTTCCTCCATCTTTGCTCAGCTAGTGGAATCCATGATGAATTCTC
ATCTCCAAGGGGTAAGCAGTTTTTAGTAAAGCCCAGTAGCTGACTTATGACTCCTTAGAAATAGCATT
GATTCCTTCCTTCTCCTGTGTTTTGTTTCCTCTAGAATGATAGAATCCATGTAGACACGATCCATTAT
CATGCTTAGGTACTGGTAAGCATGTAATGATTTTAGTTTTGTTCGCTTTAAGTTATTTGTGTCACAAA
TATCTGGGATCATATCAGAGAAATAAATAAGCACAATTAGCATTCTACTTGTTTGTTATGACTAAAGC
TAGGTTGAGGAAACAGAAAAGGACCAGAGGTCATATGAGGATGAAGATAATACTAGGAACAGCATGTT
TGGGAGAGTAACATCTGGTAGGGGTAGCAGATTGGGGGCAGAGAACAGAATTTTATAGATGGATATTT
TGGAGGCAAGTAGTTTGAGTAATGATTAGATCTAAGGTGTTTTCTCATCTGTGGGTGGCTCGAAGGAA
TAGAGGTGAAGGTCAGTTTATTTGAGAAGTTCTGGAATTATAAAACTAAGTTGAAGTCAAAGAAAGTA
TAGTAGCAAATAAATAGAATACCCTTAAAAGGAAACCAAATGAAAAATAATCGTTACTCTCACCATAT
GCTTGTGTTCTTATTAGCAAGAAATTCTTTTAACCACTGTTTTTATAATATCTTAATGAAAAAATACT
GAAGCGTATGCCATATTAAATCCCTCTCTTTATTTCTAGAAAGGGAATCAAAGGAGAAAATTCCCATT
CTGCTATACTAAAAGACCACTAAGTAAAGAGCCTATTAGTGTATGATAAATCCCATAGCAATATACAT
TATCATTTTACAGCTTCTTTGTTGAAATGAATGTTTGTATGTGTTGACCATAGAGTGGGATAAAAAGT
TGAAATTTTGTTTTGAAATATTTTAGAAATGCATAGTTGTACTGCAGTTGTGAACCTCCTTAGATTTT
TAAGGAGGCTGCTTCAAAGGATCTCATTAATAATCTTCTCAGGTGCTTACAAAGCATGTGTCTGTCAG
CAGAATTAGAGAATCACCCAACTAGAGAACAGGTTTCACAATACCCTGAGACCTATTTTGTTCATTAG
AGAGGAAAATGGCTTGTTTTGAGTCTAAGTTGACATGCTTGCTAATTTCAGCAATAAAAGCTGTTCAT
TGTGGTCAGGTTTAATTTAGAGCCTGGTAAGGTTCAGATTAAAGTTGATCAACTTACTTTTACAACAT
ACTTCTTAAATGAACTTTGAAATCTTAAAAGAAGGAAAAAAGTATAGCAAACAGTGAATAATGTATCT
AAAACTGAGAAGCAAAAAAAATCTGGTTATGTGAGAGTGAATTAAAAGAAGAACAACCCAATAAAGAT
AATCTTTGTTATATAAAAATTTCCAAGTATCGAAAAGCACGATTTTTCATGTGAGTTACACACTATAC
CGAATATATTTGTCCACTGCCACATGCATAGTCCCTAGAATAGTGCCTAGTGCTGAAAAATATTTATT
AAAATGAATGGATGAGTAAATGAATTTATGTATTTTGCCAGCCCTGTGTATTTAAAGTTCTCTGTTAA
CTTTGAGGTGAAAATTTGACTTCATCTGAGGTTTCTGGGTAAGTCCGTTTTAAAAATTCTATTGAACA
TATTCAAACATTTTAGGGTAGGCAATTCCAAAGCAACCTTTCAGCTTCCCATGTCACAGATGACCAGA
GTTTCACATTCTAACACTGGAAAACATCTTATTTCATAAAATCTACCTGCTACTATATGGTTCCTACT
TTAAAATTTGTTCAGTACTCTCCAGCTGACTTATGCCACTTACTTCAATAGCTGTCTTTGGCAATTTG
TTCCATATTTCAAACACTCTGTTGTGTAAAGAAACCATAAAAGTTAGAAACCTGAAAATTGGATTTTT
TTTCTGAGCAATCACAGCTTACAAATGTGGAAAATTTGTTAAAAGTTAGCCCCTCCAATTTTTCAATA
CAGAGAGGAGAAAGTGCCTAAAGTAGATGTACAATGTTTGGAAAAGTTTTTTGCATTATTTTACTATT
ACCAAAAGCAATTGAGTTTAAATCACAAAGCCTGTCTCCCTACCTCTTCACAGAAGAAACACTACAGA
ATGATCAAAATTTGGCCTTTCCAAAACCAAAATTCGTTAGAAAATCAGCAGGAGTCAAAGTACAGAGT
AAATAACTAAGTTTCATATAAGTTTCAGATACATTACTATCTACCACTTTCATCTCTTCATCTTCATT
GGCCTCATGTGGTAGAACATCATATTTAAAATTATACAAACTTGCTGGCTTGTTTACTAGTGTGGTTA
TTATAAGAAAAAAATGAGAAAATATAGATAAAACATCTGTCACATATTGCTTATAAACTAACAGTAAA
TATTACTTGTATTTTCCCCAATTAAAATAAAATTTACTGAGTTTTAGAACCAGAGCTAGTCAGATGCC
TTTTTTTCATAAATTTCTTCATAAATACCTCTGAGATTGTGGTCCTTAAATCTAGAGAGACTAAGATG
ACAGAGAAAATAGACACTGAAGAAAGGGAAGAATATCTTAATGATTTACATTACTACCTATAAATTAA
AAATTGTTAACTTTATTATATTTGTATTTTTATTTAAAATAGTGCTATATTAAAGTCATTTATAATAC
AGGGGAATAGGAATACTAACCTGTAATCTGATGCTCTCCAAACTTGCCTAAATCATAAAAGTTAATTA
GATAATTTATTTAAAATGCAAGATTTGCAGCCCTTTCCACTACATATTCATTAGTTCTGGAGGGAGGC
AAAAAGGTTTGGTGATTCTTACGGACAGGCAGCTTAGGAGAAAAGCTGATTTAGCTCGTCTACTTCAC
CTTTTCATTTGACAGGTGAGAAATCTCAGGGGTACAATGAAGTTAAATAAGTAATATCTCTTAAATCG
GTTCTGTGCTTTTTCTGTTTTTAAAATAAATATACCTTAATTTTGACGTCACACAGAATGATATTATA
AGTATAAATAGTTATCTATCTTTTAAATACATTGTCGTAATTCAGAATAACATTTCTTACTCAAGGCA
TTCAGACAGTGGTTTAAGTAATCCGAGGTACTCCGGAATGTCTCCATTTGAGCCTTTAAATGAAGAAA
ATCTATAGTCAAGATTTTCATTTGAAATATTTTTGATATCTAAGAATGAAACATATTTCCTGTTAAAT
TGTTTTCTATAAACCCTTATACAGTAACATCTTTTTTATTTCTAAAAGTGTTTTGGCTGGTCTCACAA
TTGTACTTTACTTTGTATTATGTAAAAGGAATACACAACGCTGAAGAACCCTGATACTAAGGGATATT
TGTTCTTACAG
SEQ ID NO: 129
AAV portion including U6-g7 expression cassette and Ex52 donor:
U6 PROMOTER, [symbol] PAM (ATTCCT), SaCas9 gRNA SCAFFOLD, donor
sequence, exon52
AGGGCCTATTTCCCATGATTCCTTCATATTTGCATATACGATACAAGGCTGTTAGAGAGATA
ATTGGAATTAATTTGACTGTAAACACAAAGATATTAGTACAAAATACGTGACGTAGAAAGTA
ATAATTTTCTTGGGTAGTTTGCAGTTTTAAAATTATGTTTTAAAATGGACTATCATATGCTTA
CCGTAACTTGAAAGTATTTCGATTTCTTGGCTTTATATATCTTGTGGAAAGGACGAAACACC
G [symbol] GTTTTAGTACTCTGGAAACAGAATCTACTAAAACAAGGCA
AAATGCCGTGTTTATCTCGTCAACTTGTTGGCGAGATTTTTTTCTAGACCCAGCTTTCTTGT
ACAAAGTTGGCGTTTAAAC [symbol] gttaatttgcgttcta
gccaccgagatacagtaacatcttttttatttctaaaagtgttttggctggtctcacaattg
tactttactttgtattatgtaaaaggaatacacaacgctgaagaaccctgatactaagggat
atttgttcttacaggcaacaatgcaggatttggaacagaggcgtccccagttggaagaactc
attaccgctgcccaaaatttgaaaaacaagaccagcaatcaagaggctagaacaatcattac
ggatcgaagtaagttttttaacaagcatgggacacacaaagcaagatgcatgacaagtttca
ataaaaacttaagttcatatatccccctcacatttataaaaataatgtgaaataattgtaaa
tgataacaattgtgctgagattttcagtccataatgttaccttttaataaatgaatgtaatt
ccattgaatagaagaaatac [symbol]
SEQ ID NO: 130
AAV portion including U6-g7 expression cassette and Superexon donor:
U6 PROMOTER, [symbol] PAM (ATTCCT), SaCas9 gRNA SCAFFOLD, donor
sequence, exon52-79 cDNA coding sequence, [symbol]
AGGGCCTATTTCCCATGATTCCTTCATATTTGCATATACGATACAAGGCTGTTAGAGAGATA
ATTGGAATTAATTTGACTGTAAACACAAAGATATTAGTACAAAATACGTGACGTAGAAAGTA
ATAATTTGTTGGGTAGTTTGGAGTTTTAAAATTATGTTTTAAAATGGAGTATCATATGGTTA
CCGTAACTTGAAAGTATTTCGATTTCTTGGCTTTATATATCTTGTGGAAAGGACGAAACACC
G [symbol] GTTTTAGTACTCTGGAAACAGAATCTACTAAAACAAGGCA
AAATGCCGTGTTTATCTCGTCAACTTGTTGGCGAGATTTTTTTCTAGACCCAGCTTTCTTGT
ACAAAGTTGGCGTTTAAAC [symbol] gttaatttgcgttcta
gccaccgagatacagtaacatcttttttatttctaaaagtgttttggctggtctcacaattg
tactttactttgtattatgtaaaaggaatacacaacgctgaagaaccctgatactaagggat
atttgttcttacaggcaacaatgcaggatttggaacagaggcgtccccagttggaagaactc
attaccgctgcccaaaatttgaaaaacaagaccagcaatcaagaggctagaacaatcattac
ggatcgaattgaaagaattcagaatcagtgggatgaagtacaagaacaccttcagaaccgga
ggcaacagttgaatgaaatgttaaaggattcaacacaatggctggaagctaaggaagaagct
gagcaggtcttaggacaggccagagccaagcttgagtcatggaaggagggtccctatacagt
agatgcaatccaaaagaaaatcacagaaaccaagcagttggccaaagacctccgccagtggc
agacaaatgtagatgtcgcaaatgacttggccctgaaacttctccgggattattctgcagat
gataccagaaaagtccacatgataacagagaatatcaatgcctcttggagaagcattcataa
aagggtgagtgagcgagaggctgctttggaagaaactcatagattactgcaacagttccccc
tggacctggaaaagtttcttgcctggcttacagaagctgaaacaactgccaatgtcctacag
gatgctacccgtaaggaaaggctcctagaagactccaagggagtaaaagagctgatgaaaca
atggcaagacctccaaggtgaaattgaagctcacacagatctttatcacaacctggatgaaa
acagccaaaaaatcctgagatccctggaaggttccgatgatgcagtcctgttacaaagacgt
ttggataacatgaacttcaagtggagtgaacttcggaaaaagtctctcaacattaggtccca
tttggaagccagttctgaccagtggaagcgtctgcacctttctctgcaggaacttctggtgt
ggctacagctgaaagatgatgaattaagccggcaggcacctattggaggcgactttccagca
gttcagaagcagaacgatgtacatagggccttcaagagggaattgaaaactaaagaacctgt
aatcatgagtactcttgagactgtacgaatatttctgacagagcagcctttggaaggactag
agaaactctaccaggagcccagagagctgcctcctgaggagagagcccagaatgtcactcgg
cttctacgaaagcaggctgaggaggtcaatactgagtgcgaaaaattgaacctgcactccgc
tcactggcagagaaaaatagatgagacccttgaaagactccaggaacttcaagaggccacgg
atgagctggacctcaagctgcgccaagctgaggtgatcaagggatcctggcagcccgtgggc
gatctcctcattgactctctccaagatcacctcgagaaagtcaaggcacttcgaggagaaat
tgcgcctctgaaagagaacgtgagccacgtcaatgaccttgctcgccagcttaccactttgg
gcattcagctctcaccgtataacctcagcactctggaagacctgaacaccagatggaagctt
ctgcaggtggccgtcgaggaccgagtcaggcagctgcatgaagcccacagggactttggtcc
agcatctcagcactttctttccacgtctgtccagggtccctgggagagagccatctcgccaa
acaaagtgccctactatatcaaccacgagactcaaacaacttgctgggaccatcccaaaatg
acagagctctaccagtctttagctgacctgaataatgtcagattctcagcttataggactgc
catgaaactccgaagactgcagaaggccctttgcttggatctcttgagcctgtcagctgcat
gtgatgccttggaccagcacaacctcaagcaaaatgaccagcccatggatatcctgcagatt
attaattgtttgaccactatttatgaccgcctggagcaagagcacaacaatttggtcaacgt
ccctctctgcgtggatatgtgtctgaactggctgctgaatgtttatgatacgggacgaacag
ggaggatccgtgtcctgtcttttaaaactggcatcatttccctgtgtaaagcacatttggaa
gacaagtacagataccttttcaagcaagtggcaagttcaacaggattttgtgaccagcgcag
gctgggcctccttctgcatgattctatccaaattccaagacagttgggtgaagttgcatcct
ttgggggcagtaacattgagccaagtgtccggagctgcttccaatttgctaataataagcca
gagatcgaagcggccctcttcctagactggatgagactggaaccccagtccatggtgtggct
gcccgtcctgcacagagtggctgctgcagaaactgccaagcatcaggccaaatgtaacatct
gcaaagagtgtccaatcattggattcaggtacaggagtctaaagcactttaattatgacatc
tgccaaagctgctttttttctggtcgagttgcaaaaggccataaaatgcactatcccatggt
ggaatattgcactccgactacatcaggagaagatgttcgagactttgccaaggtactaaaaa
acaaatttcgaaccaaaaggtattttgcgaagcatccccgaatgggctacctgccagtgcag
actgtcttagagggggacaacatcgaaactcccgttactctgatcaacttctggccagtaga
ttctgcgcctgcctcgtcccctcagctttcacacgatgatactcattcacgcattgaacatt
atgctagcaggctagcagaaatggaaaacagcaatggatcttatctaaatgatagcatctct
cctaatgagagcatagatgatgaacatttgttaatccagcattactgccaaagtttgaacca
ggactcccccctgagccagcctcgtagtcctgcccagatcttgatttccttagagagtgaag
aaagaggggagctagagagaatcctagcagatcttgaggaagaaaacaggaatctgcaagca
gaatatgaccgtctaaagcagcagcacgaacataaaggcctgtccccactgccgtcccctcc
tgaaatgatgcccacctctccccagagtccccgggatgctgagctcattgctgaggccaagc
tactgcgtcaacacaaaggccgcctggaagccaggatgcaaatcctggaagaccacaataaa
cagctggagtcacagttacacaggctaaggcagctgctggagcaaccccaggcagaggccaa
agtgaatggcacaacggtgtcctctccttctacctctctacagaggtccgacagcagtcagc
ctatgctgctccgagtggttggcagtcaaacttcggactccatgggtgaggaagatcttctc
agtcctccccaggacacaagcacagggttagaggaggtgatggagcaactcaacaactcctt
ccctagttcaagaggaagaaatacccctggaaagccaatgagagaggacacaatg [symbol] [symbol]
tcagcctcga

[symbol]

[symbol]

[symbol]

[symbol]

[symbol]

[symbol]

gtaagttttttaacaagcatgggacacacaaagcaagatgca
tgacaagtttcaataaaaacttaagttcatatatccccctcacatttataaaaataatgtga
aataattgtaaatgataacaattgtgctgagattttcagtccataatgttaccttttaataa
atgaatgtaattccattgaatagaagaaatac [symbol]
SEQ ID NO: 131
Streptccoccus pyogenes Cas9 (with D10A)
MDKKYSTGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRELENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYETVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLEDDKVMKQLKERRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
AYNKHRDKPIREQAENIIHLFTLTNLGAPAAFKYFDTTIDRKRYTSTKEVLDATLIHQSITGLYETRI
DLSQLGGD
SEQ ID NO: 132
Streptccoccus pyogenes Cas9 (with D10A, H849A)
MDKKYSTGLAiGTNSVGWAVITDEYKVPSKKFKVLGNTDRHSIKKNLIGALLFDSGETAEATRLKRTA
RRRYTRRKNRTCYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDHEEN
EDILEDIVLTLTLFEDREMIEERLKTYAHLFDDKVMKOLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTTDRKRYTSTKEVLDATLIHQSITGLYETRI
DLSQLGGD
SEQ ID NO: 133
Polynucleotide sequence of D10A mutant of Staphylococcus aureus Cas9
atgaaaagga actacattct ggggctggcc atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgo agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tcgtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctacto gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 134
Polynucleotide sequence of N580A mutant of Staphylococcus aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagaggcc
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 135
GS linker (Gly-Gly-Gly-Gly-Ser)ₙ, wherein ₙ is an integer between 0 and 10
SEQ ID NO: 136
Gly-Gly-Gly-Gly-Gly
SEQ ID NO: 137
Gly-Gly-Ala-Gly-Gly
SEQ ID NO: 138
Gly-Gly-Gly-Gly-Ser-Ser-Ser
SEQ ID NO: 139
Gly-Gly-Gly-Gly-Ala-Ala-Ala
SEQ ID NO: 140
Human p300 (with L553M mutation) protein
MAENVVEPGPPSAKRPKLSSPALSASASDGTDFGSLFDLEHDLPDELINSTELGLTNGGDINQLQTSL
GMVQDAASKHKQLSELLRSGSSPNLNMGVGGPGQVMASQAQQSSPGLGLINSMVKSPMTQAGLTSPNM
GMGTSGPNQGPTQSTGMMNSPVNQPAMGMNTGMNAGMNPGMLAAGNGQGIMPNQVMNGSIGAGRGRQN
MQYPNPGMGSAGNLLTEPLQQGSPQMGGQTGLRGPQPLKMGMMNNPNPYGSPYTQNPGQQIGASGLGL
QIQTKTVLSNNLSPFAMDKKAVPGGGMPNMGQQPAPQVQQPGLVTPVAQGMGSGAHTADPEKRKLIQQ
QLVLLLHAHKCORREQANGEVRQCNLPHCRTMKNVLNHMTHCQSGKSCQVAHCASSRQIISHWKNCTR
HDCPVCLPLKNAGDKRNQQPILTGAPVGLGNPSSLGVGQQSAPNLSTVSQIDPSSIERAYAALGLPYQ
VNQMPTOPQVQAKNQQNQQPGQSPQGMRPMSNMSASPMGVNGGVGVQTPSLLSDSMLHSAINSQNPMM
SENASVPSMGPMPTAAQPSTTGIRKQWHEDITQDLRNHLVHKLVQAIFPTPDPAALKDRRMENLVAYA
RKVEGDMYESANNRAEYYHLLAEKIYKIQKELEEKRRTRLQKQNMLPNAAGMVPVSMNPGPNMGQPQP
GMTSNGPLPDPSMIRGSVPNQMMPRITPQSGLNQFGQMSMAQPPIVPRQTPPLQHHGQLAQPGALNPP
MGYGPRMQQPSNOGQFLPQTQFPSQGMNVTNIPLAPSSGQAPVSQAQMSSSSCPVNSPIMPPGSQGSH
IHCPQLPQPALHQNSPSPVPSRTPTPHHTPPSTGAQQPPATTIPAPVPTPPAMPPGPQSQALHPPPRQ
TPTPPTTQLPQQVQPSLPAAPSADQPQQQPRSQQSTAASVPTPTAPLLPPQPATPLSQPAVSIEGQVS
NPPSTSSTEVNSQAIAEKQPSQEVKMEAKMEVDQPEPADTQPEDISESKVEDCKMESTETEERSTELK
TEIKEEEDQPSTSATQSSPAPGQSKKKIFKPEELRQALMPTLEALYRQDPESLPFRQPVDPQLLGIPD
YFDIVKSPMDLSTIKRKLDTGQYQEPWQYVDDIWLMFNNAWLYNRKTSRVYKYCSKLSEVFEQEIDPV
MQSLGYCCGRKLEFSPQTLCCYGKQLCTIPRDATYYSYQNRYHFCEKCFNEIQGESVSLGDDPSQPQT
TTNKEQFSKRKNDTLDPELFVECTECGRKMHQICVLHHEIIWPAGFVCDGCLKKSARTRKENKFSAKR
LPSTRLGTFLENRVNDFLRRQNHPESGEVTVRVVHASDKTVEVKPGMKARFVDSGEMAESFPYRTKAL
FAFEEIDGVDLCFFGMHVQEYGSDCPPPNQRRVYISYLDSVHFFRPKCLRTAVYHEILTGYLEYVKKL
GYTTGHIWACPPSEGDDYIFHCHPPDQKIPKPKRLQEWYKKMLDKAVSERIVHDYKDIFKQATEDRLT
SAKELPYFEGDFWPNVLEESIKELEQEEEERKREENTSNESTDVTKGDSKNAKKKNNKKTSKNKSSLS
RGNKKKPGMPNVSNDLSQKLYATMEKHKEVFFVIRLIAGPAANSLPPIVDPDPLIPCDLMDGRDAFLT
LARDKHLEFSSLRRAQWSTMCMLVELHTQSQDRFVYTCNECKHHVETRWHCTVCEDYDLCITCYNTKN
HDHKMEKLGLGLDDESNNQQAAATQSPGDSRRLSIQRCIQSLVHACQCRNANCSLPSCQKMKRVVQHT
KGCKRKTNGGCPICKQLIALCCYHAKHCQENKCPVPFCLNIKQKLRQQQLQHRLQQAQMLRRRMASMQ
RTGVVGQQQGLPSPTPATPTTPTGQQPTTPQTPQPTSQPQPTPPNSMPPYLPRTQAAGPVSQGKAAGQ
VTPPTPPQTAQPPLPGPPPAAVEMAMQIQRAAETQRQMAHVQIFQRPIQHQMPPMTPMAPMGMNPPPM
TRGPSGHLEPGMGPTGMQQQPPWSQGGLPQPQQLQSGMPRPAMMSVAQHGQPLNMAPQPGLGQVGISP
LKPGTVSQQALQNLLRTLRSPSSPLQQQQVLSILHANPQLLAAFIKQRAAKYANSNPQPIPGQPGMPQ
GQPGLQPPTMPGQQGVHSNPAMQNMNPMQAGVQRAGLPQQQPQQQLQPPMGGMSPQAQQMNMNHNTMP
SQFRDILRRQQMMQQQQQQGAGPGIGPGMANHNQFQQPQGVGYPPQQQQRMQHHMQQMQQGNMGQIGQ
LPQALGAEAGASLQAYQQRLLQQQMGSPVQPNPMSPQQHMLPNQAQSPHLQGQQIPNSLSNQVRSPQP
VPSPRPQSOPPHSSPSPRMQPQPSPHHVSPQTSSPHPGLVAAQANPMEQGHFASPDQNSMLSQLASNP
GMANLHGASATDLGLSTDNSDLNSNLSQSTLDIH
SEQ ID NO: 141
Human p300 Core Effector protein (aa 1048-1664 of SEQ ID NO: 140)
IFKPEELRQALMPTLEALYRQDPESLPFRQPVDPQLLGIPDYFDIVKSPMDLSTIKRKLDTGQYQEPW
QYVDDIWLMFNNAWLYNRKTSRVYKYCSKLSEVFEQEIDPVMQSLGYCCGRKLEFSPQTLCCYGKQLC
TIPRDATYYSYQNRYHFCEKCFNEIQGESVSLGDDPSQPQTTINKEQFSKRKNDTLDPELFVECTECG
RKMHQICVLHHEIIWPAGFVCDGCLKKSARTRKENKFSAKRLPSTRLGTFLENRVNDFLRRQNHPESG
EVTVRVVHASDKTVEVKPGMKARFVDSGEMAESFPYRTKALFAFEEIDGVDLCFFGMHVQEYGSDCPP
PNQRRVYISYLDSVHFFRPKCLRTAVYHEILIGYLEYVKKLGYTTGHIWACPPSEGDDYIFHCHPPDQ
KIPKPKRLQEWYKKMLDKAVSERIVHDYKDIFKQATEDRLTSAKELPYFEGDFWPNVLEESIKELEQE
EEERKREENTSNESTDVTKGDSKNAKKKNNKKTSKNKSSLSRGNKKKPGMPNVSNDLSQKLYATMEKH
KEVFFVIRLIAGPAANSLPPIVDPDPLIPCDLMDGRDAFLTLARDKHLEFSSLRRAQWSTMCMLVELH
TQSQD
SEQ ID NO: 142
VP64-dCas9-VP64 protein
RADALDDFDLDMLGSDALDDFDLDMLGSDALDDFDLDMLGSDALDDFDLDMVNPKKKRKVGRGMDKKY
SIGLAIGTNSVGWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENPINASGVDAK
AILSARLSKSRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHTANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDAIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDKVLSAYNKH
RDKPIREQAENIIHLFTLTNLGAPAAFKYFDTTTDRKRYTSTKEVLDATLIHQSITGLYETRIDLSQL
GGDSRADPKKKRKVASRADALDDFDLDMLGSDALDDFDLDMLGSDALDDEDLDMLGSDALDDFDLDML
I
SEQ ID NO: 143
VP64-dCas9-VP64 DNA
cgggctgacgcattggacgattttgatctggatatgctgggaagtgacgccctcgatgattttgacct
tgacatgcttggttcggatgcccttgatgactttgacctcgacatgctcggcagtgacgcccttgatg
atttcgacctggacatggttaaccccaagaagaagaggaaggtgggccgcggaatggacaagaagtac
tccattgggctcgccatcggcacaaacagcgtcggctgggccgtcattacggacgagtacaaggtgcc
gagcaaaaaattcaaagttctgggcaataccgatcgccacagcataaagaagaacctcattggcgccc
tcctgttcgactccggggaaaccgccgaagccacgcggctcaaaagaacagcacggcgcagatatacc
cgcagaaagaatcggatctgctacctgcaggagatctttagtaatgagatggctaaggtggatgactc
tttcttccataggctggaggagtcctttttggtggaggaggataaaaagcacgagcgccacccaatct
ttggcaatatcgtggacgaggtggcgtaccatgaaaagtacccaaccatatatcatctgaggaagaag
cttgtagacagtactgataaggctgacttgcggttgatctatctcgcgctggcgcatatgatcaaatt
tcggggacacttcctcatcgagggggacctgaacccagacaacagcgatgtcgacaaactctttatcc
aactggttcagacttacaatcagcttttcgaagagaacccgatcaacgcatccggagttgacgccaaa
gcaatcctgagcgctaggctgtccaaatcccggcggctcgaaaacctcatcgcacagctccctgggga
gaagaagaacggcctgtttggtaatcttatcgccctgtcactcgggctgacccccaactttaaatcta
acttcgacctggccgaagatgccaagcttcaactgagcaaagacacctacgatgatgatctcgacaat
ctgctggcccagatcggcgaccagtacgcagacctttttttggcggcaaagaacctgtcagacgccat
tctgctgagtgatattctgcgagtgaacacggagatcaccaaagctccgctgagcgctagtatgatca
agcgctatgatgagcaccaccaagacttgactttgctgaaggcccttgtcagacagcaactgcctgag
aagtacaaggaaattttcttcgatcagtctaaaaatggctacgccggatacattgacggcggagcaag
ccaggaggaattttacaaatttattaagcccatcttggaaaaaatggacggcaccgaggagctgctgg
attcacctgggcgaactgcacgctatcctcaggcggcaagaggatttctacccctttttgaaagataa
cagggaaaagattgagaaaatcctcacatttcggataccctactatgtaggccccctcgcccggggaa
attccagattcgcgtggatgactcgcaaatcagaagagaccatcactccctggaacttcgaggaagtc
gtggataagggggcctctgcccagtccttcatcgaaaggatgactaactttgataaaaatctgcctaa
cgaaaaggtgcttcctaaacactctctgctgtacgagtacttcacagtttataacgagctcaccaagg
tcaaatacgtcacagaagggatgagaaagccagcattcctgtctggagagcagaagaaagctatcgtg
gacctcctcttcaagacgaaccggaaagttaccgtgaaacagctcaaagaagactatttcaaaaagat
tgaatgtttcgactctgttgaaatcagcggagtggaggatcgcttcaacgcatccctgggaacgtatc
acgatctcctgaaaatcattaaagacaaggacttcctggacaatgaggagaacgaggacattcttgag
gacattgtcctcacccttacgttgtttgaagatagggagatgattgaagaacgcttgaaaacttacgc
tcatctcttcgacgacaaagtcatgaaacagctcaagaggcgccgatatacaggatgggggcggctgt
caagaaaactgatcaatgggatccgagacaagcagagtggaaagacaatcctggattttcttaagtcc
gatggatttgccaaccggaacttcatgcagttgatccatgatgactctctcacctttaaggaggacat
ccagaaagcacaagtttctggccagggggacagtcttcacgagcacatcgctaatcttgcaggtagcc
cagctatcaaaaagggaatactgcagaccgttaaggtcgtggatgaactcgtcaaagtaatgggaagg
cataagcccgagaatatcgttatcgagatggcccgagagaaccaaactacccagaagggacagaagaa
cagtagggaaaggatgaagaggattgaagagggtataaaagaactggggtcccaaatccttaaggaac
acccagttgaaaacacccagcttcagaatgagaagctctacctgtactacctgcagaacggcagggac
atgtacgtggatcaggaactggacatcaatcggctctccgactacgacgtggatgccatcgtgcccca
gtcttttctcaaagatgattctattgataataaagtgttgacaagatccgataaaaatagagggaaga
gtgataacgtcccctcagaagaagttgtcaagaaaatgaaaaattattggcggcagctgctgaacgcc
aaactgatcacacaacggaagttcgataatctgactaaggctgaacgaggtggcctgtctgagttgga
taaagccggcttcatcaaaaggcagcttgttgagacacgccagatcaccaagcacgtggcccaaattc
tcgattcacgcatgaacaccaagtacgatgaaaatgacaaactgattcgagaggtgaaagttattact
ctgaagtctaagctggtctcagatttcagaaaggactttcagttttataaggtgagagagatcaacaa
ttaccaccatgcgcatgatgcctacctgaatgcagtggtaggcactgcacttatcaaaaaatatccca
agcttgaatctgaatttgtttacggagactataaagtgtacgatgttaggaaaatgatcgcaaagtct
gagcaggaaataggcaaggccaccgctaagtacttcttttacagcaatattatgaattttttcaagac
cgagattacactggccaatggagagattcggaagcgaccacttatcgaaacaaacggagaaacaggag
aaatcgtgtgggacaagggtagggatttcgcgacagtccggaaggtcctgtccatgccgcaggtgaac
atcgttaaaaagaccgaagtacagaccggaggcttctccaaggaaagtatcctcccgaaaaggaacag
cgacaagctgatcgcacgcaaaaaagattgggaccccaagaaatacggcggattcgattctcctacag
tcgcttacagtgtactggttgtggccaaagtggagaaagggaagtctaaaaaactcaaaagcgtcaag
gaactgctgggcatcacaatcatggagcgatcaagcttcgaaaaaaaccccatcgactttctcgaggc
gaaaggatataaagaggtcaaaaaagacctcatcattaagcttcccaagtactctctctttgagcttg
aaaacggccggaaacgaatgctcgctagtgcgggcgagctgcagaaaggtaacgagctggcactgccc
tctaaatacgttaatttcttgtatctggccagccactatgaaaagctcaaagggtctcccgaagataa
tgagcagaagcagctgttcgtggaacaacacaaacactaccttgatgagatcatcgagcaaataagcg
aattctccaaaagagtgatcctcgccgacgctaacctcgataaggtgctttctgcttacaataagcac
agggataagcccatcagggagcaggcagaaaacattatccacttgtttactctgaccaacttgggcgc
gcctgcagccttcaagtacttcgacaccaccatagacagaaagcggtacacctctacaaaggaggtcc
tggacgccacactgattcatcagtcaattacggggctctatgaaacaagaatcgacctctctcagctc
ggtggagacagcagggctgaccccaagaagaagaggaaggtggctagccgcgccgacgcgctggacga
tttcgatctcgacatgctgggttctgatgccctcgatgactttgacctggatatgttgggaagcgacg
cattggatgactttgatctggacatgctcggctccgatgctctggacgatttcgatctcgatatgtta
atc
SEQ ID NO: 144
Protein sequence for VPH
DALDDFDLDMLGSDALDDFDLDMLGSDALDDFDLDMLGSDALDDFDLDMLGSLPSASVEFEGSGGPSG
QISNQALALAPSSAPVLAQTMVPSSAMVPLAQPPAPAPVLTPGPPQSLSAPVPKSTQAGEGTLSEALL
HLQFDADEDLGALLGNSTDPGVFTDLASVDNSEFQQLLNQGVSMSHSTAEPMLMEYPEAITRLVTGSQ
RPPDPAPTPLGTSGLPNGLSGDEDFSSIADMDFSALLSQISSSGQGGGGSGFSVDTSALLDLFSPSVT
VPDKSLPDLDSSLASIQELLSPQEPPRPPEAENSSPDSGKQLVHYTAQPLFLLDPGSVDTGSNDLPVL
FELGEGSYFSEGDGFAEDPTISLLTGSEPPKAKDPTVS
SEQ ID NO: 145
DNA sequence for VPH
gatgctttagacgattttgacttagatatgcttggttcagacgcgttagacgacttcgacctagacat
gttaggctcagatgcattggacgacttcgatttagatatgttgggctccgatgccctagatgactttg
atctagatatgctagggtcactacccagcgccagcgtcgagttcgaaggcagcggcgggccttcaggg
cagatcagcaaccaggccctggctctggcccctagctccgctccagtgctggcccagactatggtgcc
ctctagtgctatggtgcctctggcccagccacctgctccagcccctgtgctgaccccaggaccacccc
agtcactgagcgccccagtgcccaagtctacacaggccggcgaggggactctgagtgaagctctgctg
cacctgcagttcgacgctgatgaggacctgggagctctgctggggaacagcaccgatcccggagtgtt
cacagatctggcctccgtggacaactctgagtttcagcagctgctgaatcagggcgtgtccatgtctc
atagtacagccgaaccaatgctgatggagtaccccgaagccattacccggctggtgaccggcagccag
cggccccccgaccccgctccaactcccctgggaaccagcggcctgcctaatgggctgtccggagatga
agacttctcaagcatcgctgatatggactttagtgccctgctgtcacagatttcctctagtgggcagg
gaggaggtggaagcggcttcagcgtggacaccagtgccctgctggacctgttcagcccctcggtgacc
gtgcccgacatgagcctgcctgaccttgacagcagcctggccagtatccaagagctcctgtctcccca
ggagccccccaggcctcccgaggcagagaacagcagcccggattcagggaagcagctggtgcactaca
cagcgcagccgctgttcctgctggaccccggctccgtggacaccgggagcaacgacctgccggtgctg
tttgagctgggagagggctcctacttctccgaaggggacggcttcgccgaggaccccaccatctccct
gcf.gacaggctcggagcctcccaaagccaaggaccccactgtctcc
SEQ ID NO: 146
Protein sequence for VPR
DALDDFDLDMLGSDALDDFDLDMLGSDALDDFDLDMLGSDALDDFDLDMLGSPKKKRKVGSQYLPDTD
DRHRIEEKRKRTYETFKSIMKKSPFSGPTDPRPPPRRIAVPSRSSASVPKPAPQPYPFTSSLSTINYD
EFPTMVFPSGQISQASALAPAPPQVLPQAPAPAPAPAMVSALAQAPAPVPVLAPGPPQAVAPPAPKPT
QAGEGTLSEALLQLQFDDEDLGALLGNSTDPAVFTDLASVDNSEFQQLLNQGIPVAPHTTEPMLMEYP
EAITRLVTGAQRPPDPAPAPLGAPGLPNGLLSGDEDFSSTADMDFSALLSQISSGSGSGSRDSREGMF
LPKPEAGSAISDVFEGREVCQPKRIRPFHPPGSPWANRPLPASLAPTPTGPVHEPVGSLTPAPVPQPL
DPAPAVTPEASHLLEDPDEETSQAVKALREMADTVIPQKEEAAICGQMDLSHPPPRGHLDELTTTLES
MTEDLNLDSPLTPELNEILDTELNDECLLHAMHISTGLSIFDTSLF
SEQ ID NO: 147
DNA sequence for VPR
gatgctttagacgattttgacttagatatgcttggttcagacgcgttagacgacttcgacctagacat
gttaggctcagatgcattggacgacttcgatttagatatgttgggctccgatgccctagatgactttg
atctagatatgctaggtagtcccaaaaagaagaggaaagtgggatcccagtatctgcccgacacagat
gatagacaccgaatcgaagagaaacgcaagcgaacgtatgaaaccttcaaatcgatcatgaagaaatc
gcccttctcgggtccgaccgatcccaggcccccaccgagaaggattgcggtcccgtcccgctcgtcgg
ccagcgtgccgaagcctgcgccgcagccctaccccttcacgtcgagcctgagcacaatcaattatgac
gagttcccgacgatggtgttcccctcgggacaaatctcacaagcctcggcgctcgcaccagcgcctcc
ccaagtccttccgcaagcgcctgccccagcgcctgcaccggcaatggtgtccgccctcgcacaggccc
ctgcgcccgtccccgtgctcgcgcctggaccgccccaggcggtcgctccaccggctccgaagccgacg
caggccggagagggaacactctccgaagcacttcttcaactccagtttgatgacgaggatcttggagc
actccttggaaactcgacagaccctgcggtgtttaccgacctcgcgtcagtagataactccgaatttc
agcagcctttgaaccagggtatcccggtcgcgccacatacaacggagcccatgttgatggaatacccc
gaagcaatcacgagacttgtgacgggagcgcagcggcctcccgatcccgcacccgcacctttgggggc
acctggcctccctaacggacttttgagcggcgacgaggatttctcctccatcgccgatatggatttct
cagccttgctgtcacagatttccagcggctctggcagcggcagccgggattccagggaagggatgttt
ttgccgaagcctgaggccggctccgctattagtgacgtgtttgagggccgcgaggtgtgccagccaaa
acgaatccggccatttcatcctccaggaagtccatgggccaaccgcccactccccgccagcctcgcac
caacaccaaccggtccagtacatgagccagtcgggtcactgaccccggcaccagtccctcagccactg
gatccagcgcccgcagtgactcccgaggccagtcacctgttggaggatcccgatgaagagacgagcca
ggctgtcaaagcccttcgggagatggccgatactgtgattccccagaaggaagaggctgcaatctgtg
gccaaatggacctttcccatccgcccccaaggggccatctggatgagctgacaaccacacttgagtcc
atgaccgaggatctgaacctggactcacccctgaccccggaattgaacgagattctggataccttcct
gaacgacgagtgcctcttgcatgccatgcatatcagcacaggactgtccatcttcgacacatctctgt
tt
SEQ ID NO: 148
Polynucleotide sequence encoding Streptccoccus pyogenes dCas9-KRAB
atggactacaaagaccatgacggtgattataaagatcatgacatcgattacaaggatgacgatgacaa
gatggcccccaagaagaagaggaaggtgggccgcggaatggacaagaagtactccattgggctcgcca
tcggcacaaacagcgtcggctgggccgtcattacggacgagtacaaggtgccgagcaaaaaattcaaa
gttctgggcaataccgatcgccacagcataaagaagaacctcattggcgccctcctgttcgactccgg
ggaaaccgccgaagccacgcggctcaaaagaacagcacggcgcagatatacccgcagaaagaatcgga
tctgctacctgcaggagatctttagtaatgagatggctaaggtggatgactctttcttccataggctg
gaggagtcctttttggtggaggaggataaaaagcacgagcgccacccaatctttggcaatatcgtgga
cgaggtggcgtaccatgaaaagtacccaaccatatatcatctgaggaagaagcttgtagacagtactg
ataaggctgacttgcggttgatctatctcgcgctggcgcatatgatcaaatttcggggacacttcctc
atcgagggggacctgaacccagacaacagcgatgtcgacaaactctttatccaactggttcagactta
caatcagcttttcgaagagaacccgatcaacgcatccggagttgacgccaaagcaatcctgagcgcta
ggctgtccaaatcccggcggctcgaaaacctcatcgcacagctccctggggagaagaagaacggcctg
tttggtaatcttatcgccctgtcactcgggctgacccccaactttaaatctaacttcgacctggccga
agatgccaagcttcaactgagcaaagacacctacgatgatgatctcgacaatctgctggcccagatcg
gcgaccagtacgcagacctttttttggcggcaaagaacctgtcagacgccattctgctgagtgatatt
ctgcgagtgaacacggagatcaccaaagctccgctgagcgctagtatgatcaagcgctatgatgagca
ccaccaagacttgactttgctgaaggcccttgtcagacagcaactgcctgagaagtacaaggaaattt
tcttcgatcagtctaaaaatggctacgccggatacattgacggcggagcaagccaggaggaattttac
aaatttattaagcccatcttggaaaaaatggacggcaccgaggagctgctggtaaagcttaacagaga
agatctgttgcgcaaacagcgcactttcgacaatggaagcatcccccaccagattcacctgggcgaac
tgcacgctatcctcaggcggcaagaggatttctacccctttttgaaagataacagggaaaagattgag
aaaatcctcacatttaggataccctactatgtaggccccctcgcccggggaaattccagattcgcgtg
gatgactcgcaaatcagaagagaccatcactccctggaacttcgaggaagtcgtggataagggggcct
ctgcccagtccttcatcgaaaggatgactaactttgataaaaatctgcctaacgaaaaggtgcttcct
aaacactctctgctgtacgagtacttcacagtttataacgagctcaccaaggtcaaatacgtcacaga
agggatgagaaagccagcattcctgtctggagagcagaagaaagctatcgtggacctcctcttcaaga
cgaaccggaaagttaccgtgaaacagctcaaagaagactatttcaaaaagattgaatgtttcgactct
gttgaaatcagcggagtggaggatcgcttcaacgcatccctgggaacgtatcacgatctcctgaaaat
cattaaagacaaggacttcctggacaatgaggagaacgaggacattcttgaggacattgtcctcaccc
ttacgttgtttgaagatagggagatgattgaagaacgcttgaaaacttacgctcatctcttcgacgac
aaagtcatgaaacagctcaagaggcgccgatatacaggatgggggcggctgtcaagaaaactgatcaa
tgggatccgagacaagcagagtggaaagacaatcctggattttcttaagtccgatggatttgccaacc
ggaacttcatgcagttgatccatgatgactctctcacctttaaggaggacatccagaaagcacaagtt
tctggccagggggacagtcttcacgagcacatcgctaatcttgcaggtagcccagctatcaaaaaggg
aatactgcagaccgttaaggtcgtggatgaactcgtcaaagtaatgggaaggcataagcccgagaata
tcgttatcgagatggcccgagagaaccaaactacccagaagggacagaagaacagtagggaaaggatg
aagaggattgaagagggtataaaagaactggggtcccaaatccttaaggaacacccagttgaaaacac
ccagcttcagaatgagaagctctacctgtactacctgcagaacggcagggacatgtacgtggatcagg
aactggacatcaatcggctctccgactacgacgtggatgccatcgtgccccagtcttttctcaaagat
gattctattgataataaagtgttgacaagatccgataaaaatagagggaagagtgataacgtcccctc
agaagaagttgtcaagaaaatgaaaaattattggcggcagctgctgaacgccaaactgatcacacaac
ggaagttcgataatctgactaaggctgaacgaggtggcctgtctgagttggataaagccggcttcatc
aaaaggcagcttgttgagacacgccagatcaccaagcacgtggcccaaattctcgattcacgcatgaa
caccaagtacgatgaaaatgacaaactgattcgagaggtgaaagttattactctgaagtctaagctgg
tctcagatttcagaaaggactttcagttttataaggtgagagagatcaacaattaccaccatgcgcat
gatgcctacctgaatgcagtggtaggcactgcacttatcaaaaaatatcccaagcttgaatctgaatt
tgtttacggagactataaagtgtacgatgttaggaaaatgatcgcaaagtctgagcaggaaataggca
aggccaccgctaagtacttcttttacagcaatattatgaattttttcaagaccgagattacactggcc
aatggagagattcggaagcgaccacttatcgaaacaaacggagaaacaggagaaatcgtgtgggacaa
gggtagggatttcgcgacagtccggaaggtcctgtccatgccgcaggtgaacatcgttaaaaagaccg
aagtacagaccggaggcttctccaaggaaagtatcctcccgaaaaggaacagcgacaagctgatcgca
cgcaaaaaagattgggaccccaagaaatacggcggattcgattctcctacagtcgcttacagtgtact
ggttgtggccaaagtggagaaagggaagtctaaaaaactcaaaagcgtcaaggaactgctgggcatca
caatcatggagcgatcaagcttcgaaaaaaaccccatcgactttctcgaggcgaaaggatataaagag
gtcaaaaaagacctcatcattaagcttcccaagtactctctctttgagcttgaaaacggccggaaacg
aatgctcgctagtgcgggcgagctgcagaaaggtaacgagctggcactgccctctaaatacgttaatt
tcttgtatctggccagccactatgaaaagctcaaagggtctcccgaagataatgagcagaagcagctg
ttcgtggaacaacacaaacactaccttgatgagatcatcgagcaaataagcgaattctccaaaagagt
gatcctcgccgacgctaacctcgataaggtgctttctgcttacaataagcacagggataagcccatca
gggagcaggcagaaaacattatccacttgtttactctgaccaacttgggcgcgcctgcagccttcaag
tacttcgacaccaccatagacagaaagcggtacacctctacaaaggaggtcctggacgccacactgat
tcatcagtcaattacggggctctatgaaacaagaatcgacctctctcagctcggtggagacagcaggg
ctgaccccaagaagaagaggaaggtggctagcgatgctaagtcactgactgcctggtcccggacactg
gtgaccttcaaggatgtgtttgtggacttcaccagggaggagtggaagctgctggacactgctcagca
gatcctgtacagaaatgtgatgctggagaactataagaacctggtttccttgggttatcagcttacta
agccagatgtgatcctccggttggagaagggagaagagccctggctggtggagagagaaattcaccaa
gagacccatcctgattcagagactgcatttgaaatcaaatcatcagttccgaaaaagaaacgcaaagt
ttga
SEQ ID NO: 149
Polypeptide sequence of Streptccoccus pyogenes dCas9-KRAB protein
MDYKDHDGDYKDHDIDYKDDDDKMAPKKKRKVGRGMDKKYSIGLAIGTNSVGWAVITDEYKVPSKKFK
VLGNTDRHSIKKNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDAILLSDI
LRVNTEITKAPLSASMIKRYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKTYAHLFDD
KVMKQLKRRRYTGWGRLSRKLINGIRDKQSGKTILDFLKSDGFANRNFMQLIHDDSLTFKEDIQKAQV
SGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDAIVPQSFLKD
DSIDNKVLTRSDKNRGKSDNVPSEEVVKKMKNYWROLLNAKLITQRKFDNLTKAERGGLSELDKAGFI
KRQLVETRQITKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHLFTLTNLGAPAAFK
YFDTTIDRKRYTSTKEVLDATLIHQSITGLYETRIDLSQLGGDSRADPKKKRKVASDAKSLTAWSRTL
VTFKDVFVDFTREEWKLLDTAQQILYRNVMLENYKNLVSLGYQLTKPDVILRLEKGEEPWLVEREIHQ
ETHPDSETAFEIKSSVPKKKRKV
SEQ ID NO: 150
Polynucleotide sequence of Staphylococcus aureus dCas9-KRAB protein
atggccccaaagaagaagcggaaggtcggtatccacggagtcccagcagccaagcggaactacatcct
gggcctggccatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaagccagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggCggccacgaaaaaggccggccaggcaaaaaagaaaaagggatccgat
gctaagtcactgactgcctggtcccggacactggtgaccttcaaggatgtgtttgtggacttcaccag
ggaggagtggaagctgctggacactgctcagcagatcctgtacagaaatgtgatgctggagaactata
agaacctggtttccttgggttatcagcttactaagccagatgtgatcctccggttggagaagggagaa
gagccctggctggtggagagagaaattcaccaagagacccatcctgattcagagactgcatttgaaat
caaatcatcagttccgaaaaagaaacgcaaagtt
SEQ ID NO: 151
Polypeptide sequence of Staphylococcus aureus dCas9-KRAB protein
MAPKKKRKVGIHGVPAAKRNYILGLAIGITSVGYGIIDYETRDVIDAGVRLFKEANVENNEGRRSKRG
ARRLKRRRRHRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYAYNADLY
NALNDLNNLVITRDENEKLEYYEKFQIIENVFKQKKKPTLKQTAKEILVNEEDIKGYRVTSTGKPEFT
NLKVYHDIKDITARKEIIENAELLDQIAKILTIYQSSEDIQEELTNLNSELTQEEIEQISNLKGYTGT
HNLSLKAINLILDELWHTNDNQIAIFNRLKLVPKKVDLSQQKEIPTTLVDDFILSPVVKRSFIQSIKV
INAIIKKYGLPNDIIIELAREKNSKDAQKMINEMQKRNRQTNERIEEIIRTTGKENAKYLIEKIKLHD
MQEGKCLYSLEAIPLEDLLNNPFNYEVDHIIPRSVSFDNSENNKVLVKQEEASKKGNRTPFQYLSSSD
SKISYETFKKHILNLAKGKGRISKTKKEYLLEERDINRESVQKDFINRNLVDTRYATRGLMNLLRSYF
RVNNLDVKVKSINGGFTSFLRRKWKFKKERNKGYKHHAEDALIIANADFIFKEWKKLDKAKKVMENQM
FEEKQAESMPEIETEQEYKEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKPYRFDVYLDNGVYKFVTVKNLDVIKK
ENYYEVNSKCYEEAKKLKKISNQAEFTASFYNNDLIKINGELYRVIGVNNDLLNRIEVNMIDITYREY
LENMNDKRPPRIIKTIASKTQSIKKYSTDILGNLYEVKSKKHPQIIKKGKRPAATKKAGQAKKKKGSD
AKSLTAWSRTLVTFKDVFVDFTREEWKLLDTAQQILYRNVMLENYKNLVSLGYQLTKPDVILRLEKGE
EPWLVEREIHQETHPDSETAFEIKSSVPKKKRKV
SEQ ID NO: 152
Polynucleotide sequence of Tet1CD
CTGCCCACCTGCAGCTGTCTTGATCGAGTTATACAAAAAGACAAAGGCCCATATTATACACACCTTGG
GGCAGGACCAAGTGTTGCTGCTGTCAGGGAAATCATGGAGAATAGGTATGGTCAAAAAGGAAACGCAA
TAAGGATAGAAATAGTAGTGTACACCGGTAAAGAAGGGAAAAGCTCTCATGGGTGTCCAATTGCTAAG
TGGGTTTTAAGAAGAAGCAGTGATGAAGAAAAAGTTCTTTGTTTGGTCCGGCAGCGTACAGGCCACCA
CTGTCCAACTGCTGTGATGGTGGTGCTCATCATGGTGTGGGATGGCATCCCTCTTCCAATGGCCGACC
GGCTATACACAGAGCTCACAGAGAATCTAAAGTCATACAATGGGCACCCTACCGACAGAAGATGCACC
CTCAATGAAAATCGTACCTGTACATGTCAAGGAATTGATCCAGAGACTTGTGGAGCTTCATTCTCTTT
TGGCTGTTCATGGAGTATGTACTTTAATGGCTGTAAGTTTGGTAGAAGCCCAAGCCCCAGAAGATTTA
GAATTGATCCAAGCTCTCCCTTACATGAAAAAAACCTTGAAGATAACTTACAGAGTTTGGCTACACGA
TTAGCTCCAATTTATAAGCAGTATGOTCCAGTAGCTTACCAAAATCAGGTGGAATATGAAAATGTTGC
CCGAGAATGTCGGCTTGGCAGCAAGGAAGGTCGACCCTTCTCTGGGGTCACTGCTTGCCTGGACTTCT
GTGCTCATCCCCACAGGGACATTCACAACATGAATAATGGAAGCACTGTGGTTTGTACCTTAACTCGA
GAAGATAACCGOTCTTTGGGTGTTATTCCTCAAGATGAGCAGCTCCATGTGCTACCTCTTTATAAGCT
TTCAGACACAGATGAGITTGGCTCCAAGGAAGGAATGGAAGCCAAGATCAAATCTGGGGCCATCGAGG
TCCTGGCACCCCGCCGCAAAAAAAGAACGTGTTTCACTCAGCCTGTTCCCCGTTCTGGAAAGAAGAGG
GCTGCGATGATGACAGAGGTTCTTGCACATAAGATAAGGGCAGTGGAAAAGAAACCTATTCCCCGAAT
CAAGCGGAAGAATAACTCAACAACAACAAACAACAGTAAGCCTTCGTCACTGCCAACCTTAGGGAGTA
ACACTGAGACCGTGCAACCTGAAGTAAAAAGTGAAACCGAACCCCATTTTATCTTAAAAAGTTCAGAC
AACACTAAAACTTATTCGCTGATGCCATCCGCTCCTCACCCAGTGAAAGAGGCATCTCCAGGCTTCTC
CTGGTCCCCGAAGACTGCTTCAGCCACACCAGCTCCACTGAAGAATGACGCAACAGCCTCATGCGGGT
TTTCAGAAAGAAGCAGCACTCCCCACTGTACGATGCCTTCGGGAAGACTCAGTGGTGCCAATGCTGCA
GCTGCTGATGGCCCTGGCATTTCACAGCTTGGCGAAGTGGCTCCTCTCCCCACCCTGTCTGCTCCTGT
GATGGAGCCCCTCATTAATTCTGAGCCTTCCACTGGTGTGACTGAGCCGCTAACGCCTCATCAGCCAA
ACCACCAGCCCTCCTTCCTCACCTCTCCTCAAGACCTTGCCTCTTCTCCAATGGAAGAAGATGAGCAG
CATTCTGAAGCAGATGAGCCTCCATCAGACGAACCCCTATCTGATGACCCCCTGTCACCTGCTGAGGA
GAAATTGCCCCACATTGATGAGTATTGGTCAGACAGTGAGCACATCITTTTGGATGCAAATATTGGTG
GGGTGGCCATCGCACCTGCTCACGGCTCGGTTTTGATTGAGTGTGCCCGGCGAGAGCTGCACGCTACC
ACTCCTGTTGAGCACCCCAACCGTAATCATCCAACCCGCCTCTCCCTTGTCTTTTACCAGCACAAAAA
CCTAAATAAGCCCCAACATGGTTTTGAACTAAACAAGATTAAGTTTGAGGCTAAAGAAGCTAAGAATA
AGAAAATGAAGGCCTCAGAGCAAAAAGACCAGGCAGCTAATGAAGGTCCAGAACAGTCCTCTGAAGTA
AATGAATTGAACCAAATTCCTTCTCATAAAGCATTAACATTAACCCATGACAATGTTGTCACCGTGTC
CCCTTATGCTCTCACACACGTTGCGGGGCCCTATAACCATTGGGTC
SEQ ID NO: 153
Polypeptide sequence of Tet1CD
LPTCSCLDRVIQKDKGPYYTHLGAGPSVAAVREIMENRYGQKGNAIRIEIVVYTGKEGKSSHGCPIAK
WVLRRSSDEEKVLCLVRQRTGHHCPTAVMVVLIMVWDGIPLPMADRLYTELTENLKSYNGHPTDRRCT
LNENRTCTCQGIDPETCGASFSFGCSWSMYFNGCKFGRSPSPRRFRIDPSSPLHEKNLEDNLQSLATR
LAPIYKQYAPVAYQNQVEYENVARECRLGSKEGRPFSGVTACLDFCAHPHRDIHNMNNGSTVVCTLTR
EDNRSLGVIPQDEQLHVLPLYKLSDTDEFGSKEGMEAKIKSGAIEVLAPREKKRTCFTQPVPRSGKKR
AAMMTEVLAHKIRAVEKKPIPRIKRKNNSTTTNNSKPSSLPTLGSNTETVQPEVKSETEPHFILKSSD
NTKTYSLMPSAPHPVKEASPGFSWSPKTASATPAPLKNDATASCGFSERSSTPHCTMPSGRLSGANAA
AADGPGISQLGEVAPLPTLSAPVMEPLINSEPSTGVTEPLTPHQPNHQPSFLTSPQDLASSPMEEDEQ
HSEADEPPSDEPLSDDPLSPAEEKLPHIDEYWSDSEHIFLDANIGGVAIAPAHGSVLIECARRELHAT
TPVEHPNRNHPTRLSLVFYQHKNLNKPQHGFELNKIKFEAKEAKNKKMKASEQKDQAANEGPEQSSEV
NELNQIPSHKALTLTHDNVVTVSPYALTHVAGPYNHWV
SEQ ID NO: 154
DNA encoding Super exon (exons 45-79) of human DMD
gaactccaggatggcattgggcagcggcaaactgttgtcagaacattgaatgcaactggggaagaaat
aattcagcaatcctcaaaaacagatgccagtattctacaggaaaaattgggaagcctgaatctgcggt
ggcaggaggtctgcaaacagctgtcagacagaaaaaagaggctagaagaacaaaagaatatcttgtca
gaatttcaaagagatttaaatgaatttgttttatggttggaggaagcagataacattgctagtatccc
acttgaacctggaaaagagcagcaactaaaagaaaagcttgagcaagtcaagttactggtggaagagt
tgcccctgcgccagggaattctcaaacaattaaatgaaactggaggacccgtgcttgtaagtgctccc
ataagcccagaagagcaagataaacttgaaaataagctcaagcagacaaatctccagtggataaaggt
ttccagagctttacctgagaaacaaggagaaattgaagctcaaataaaagaccttgggcagcttgaaa
aaaagcttgaagaccttgaagagcagttaaatcatctgctgctgtggttatctcctattaggaatcag
ttggaaatttataaccaaccaaaccaagaaggaccatttgacgttcaggaaactgaaatagcagttca
agctaaacaaccggatgtggaagagattttgtctaaagggcagcatttgtacaaggaaaaaccagcca
ctcagccagtgaagaggaagttagaagatctgagctctgagtggaaggcggtaaaccgtttacttcaa
gagctgagggcaaagcagcctgacctagctcctggactgaccactattggagcctctcctactcagac
tgttactctggtgacacaacctgtggttactaaggaaactgccatctccaaactagaaatgccatctt
ccttgatgttggaggtacctgctctggcagatttcaaccgggcttggacagaacttaccgactggctt
tctctgcttgatcaagttataaaatcacagagggtgatggtgggtgaccttgaggatatcaacgagat
gatcatcaagcagaaggcaacaatgcaggatttggaacagaggcgtccccagttggaagaactcatta
ccgctgcccaaaatttgaaaaacaagaccagcaatcaagaggctagaacaatcattacggatcgaatt
gaaagaattcagaatcagtgggatgaagtacaagaacaccttcagaaccggaggcaacagttgaatga
aatgttaaaggattcaacacaatggctggaagctaaggaagaagctgagcaggtcttaggacaggcca
gagccaagcttgagtcatggaaggagggtccctatacagtagatgcaatccaaaagaaaatcacagaa
accaagcagttggccaaagacctccgccagtggcagacaaatgtagatgtggcaaatgacttggccct
gaaacttctccgggattattctgcagatgataccagaaaagtccacatgataacagagaatatcaatg
cctcttggagaagcattcataaaagggtgagtgagcgagaggctgctttggaagaaactcatagatta
ctgcaacagttccccctggacctggaaaagtttcttgcctggcttacagaagctgaaacaactgccaa
tgtcctacaggatgctacccgtaaggaaaggctcctagaagactccaagggagtaaaagagctgatga
aacaatggcaagacctccaaggtgaaattgaagctcacacagatgtttatcacaacctggatgaaaac
agccaaaaaatcctgagatccctggaaggttccgatgatgcagtcctgttacaaagacgtttggataa
catgaacttcaagtggagtgaacttcggaaaaagtctctcaacattaggtcccatttggaagccagtt
ctgaccagtggaagcgtctgcacctttctctgcaggaacttctggtgtggctacagctgaaagatgat
gaattaagccggcaggcacctattggaggcgactttccagcagttcagaagcagaacgatgtacatag
ggccttcaagagggaattgaaaactaaagaacctgtaatcatgagtactcttgagactgtacgaatat
ttctgacagagcagcctttggaaggactagagaaactctaccaggagcccagagagctgcctcctgag
gagagagcccagaatgtcactcggcttctacgaaagcaggctgaggaggtcaatactgagtgggaaaa
attgaacctgcactccgctgactggcagagaaaaatagatgagacccttgaaagactccaggaacttc
aagaggccacggatgagctggacctcaagctgcgccaagctgaggtgatcaagggatcctggcagccc
gtgggcgatctcctcattgactctctccaagatcacctcgagaaagtcaaggcacttcgaggagaaat
tgcgcctctgaaagagaacgtgagccacgtcaatgaccttgctcgccagcttaccactttgggcattc
agctctcaccgtataacctcagcactctggaagacctgaacaccagatggaagcttctgcaggtggcc
gtcgaggaccgagtcaggcagctgcatgaagcccacagggactttggtccagcatctcagcactttct
ttccacgtctgtccagggtccctgggagagagccatctcgccaaacaaagtgccctactatatcaacc
acgagactcaaacaacttgctgggaccatcccaaaatgacagagctctaccagtctttagctgacctg
aataatgtcagattctcagcttataggactgccatgaaactccgaagactgcagaaggccctttgctt
ggatctcttgagcctgtcagctgcatgtgatgccttggaccagcacaacctcaagcaaaatgaccagc
ccatggatatcctgcagattattaattgtttgaccactatttatgaccgcctggagcaagagcacaac
aatttggtcaacgtccctctctgcgtggatatgtgtctgaactggctgctgaatgtttatgatacggg
acgaacagggaggatccgtgtcctgtcttttaaaactggcatcatttccctgtgtaaagcacatttgg
aagacaagtacagataccttttcaagcaagtggcaagttcaacaggattttgtgaccagcgcaggctg
ggcctccttctgcatgattctatccaaattccaagacagttgggtgaagttgcatcctttgggggcag
taacattgagccaagtgtccggagctgcttccaatttgctaataataagccagagatcgaagcggccc
tcttcctagactggatgagactggaaccccagtccatggtgtggctgcccgtcctgcacagagtggct
gctgcagaaactgccaagcatcaggccaaatgtaacatctgcaaagagtgtccaatcattggattcag
gtacaggagtctaaagcactttaattatgacatctgccaaagctgctttttttctggtcgagttgcaa
aaggccataaaatgcactatcccatggtggaatattgcactccgactacatcaggagaagatgttcga
gactttgccaaggtactaaaaaacaaatttcgaaccaaaaggtattttgcgaagcatccccgaatggg
ctacctgccagtgcagactgtcttagagggggacaacatggaaactcccgttactctgatcaacttct
ggccagtagattctgcgcctgcctcgtcccctcagctttcacacgatgatactcattcacgcattgaa
cattatgctagcaggctagcagaaatggaaaacagcaatggatcttatctaaatgatagcatctctcc
taatgagagcatagatgatgaacatttgttaatccagcattactgccaaagtttgaaccaggactccc
ccctgagccagcctcgtagtcctgcccagatcttgatttccttagagagtgaggaaagaggggagcta
gagagaatcctagcagatcttgaggaagaaaacaggaatctgcaagcagaatatgaccgtctaaagca
gcagcacgaacataaaggcctgtccccactgccgtcccctcctgaaatgatgcccacctctccccaga
gtccccgggatgctgagctcattgctgaggccaagctactgcgtcaacacaaaggccgcctggaagcc
aggatgcaaatcctggaagaccacaataaacagctggagtcacagttacacaggctaaggcagctgct
ggagcaaccccaggcagaggccaaagtgaatggcacaacggtgtcctctccttctacctctctacaga
ggtccgacagcagtcagcctatgctgctccgagtggttggcagtcaaacttcggactccatgggtgag
gaagatcttctcagtcctccccaggacacaagcacagggttagaggaggtgatggagcaactcaacaa
ctccttccctagttcaagaggaagaaatacccctggaaagccaatgagagaggacacaatgtag
SEQ ID NO: 155
Donor sequence including super exon (exons 45-79) of human DMD
tgggcatgtcagtttcatagggaaattttcacatggagcttttgtatttctttctttgccagtacaac
tgcatgtggtagcacactgtttaatcttttctcaaataaaaagacatggggcttcatttttgttttgc
ctttttggtatcttacaggaactccaggatggcattgggcagcggcaaactgttgtcagaacattgaa
tgcaactggggaagaaataattcagcaatcctcaaaaacagatgccagtattctacaggaaaaattgg
gaagcctgaatctgcggtggcaggaggtctgcaaacagctgtcagacagaaaaaagaggctagaagaa
caaaagaatatcttgtcagaatttcaaagagatttaaatgaatttgttttatggttggaggaagcaga
taacattgctagtatcccacttgaacctggaaaagagcagcaactaaaagaaaagcttgagcaagtca
agttactggtggaagagttgcccctgcgccagggaattctcaaacaattaaatgaaactggaggaccc
gtgcttgtaagtgctcccataagcccagaagagcaagataaacttgaaaataagctcaagcagacaaa
tctccagtggataaaggtttccagagctttacctgagaaacaaggagaaattgaagctcaaataaaag
accttcggcagcttgaaaaaaagcttgaagaccttgaagagcagttaaatcatctgctgctgtggtta
tctcctattaggaatcagttcgaaatttataaccaaccaaaccaagaaggaccatttgacgttcagga
aactgaaatagcagttcaagctaaacaacccgatgtggaagagattttgtctaaagggcagcatttgt
acaaggaaaaaccagccactcagccagtgaagaggaagttagaagatctgagctctgagtggaaggcg
gtaaaccgtttacttcaagagctgagggcaaagcagcctgacctagctcctggactgaccactattgg
agcctctcctactcagactgttactctggtgacacaacctgtggttactaaggaaactgccatctcca
aactagaaatgccatcttccttgatgttggaggtacctgctctggcagatttcaaccgggcttggaca
gaacttaccgactggctttctctgcttgatcaagttataaaatcacagagggtaatggtgggtgacct
tgaggatatcaacgagatgatcatcaagcagaaggcaacaatgcaggatttggaacagaggcgtcccc
agttggaagaactcattaccgctgcccaaaatttgaaaaacaagaccagcaatcaagaggctagaaca
atcattacggatcgaattgaaagaattcagaatcagtgcgatgaagtacaagaacaccttcagaaccg
gaggcaacagttgaatgaaatgttaaaggattcaacacaatggctggaagctaaggaagaagctgagc
aggtcttaggacaggccagagccaagcttgagtcatggaaggagggtccctatacagtagatgcaatc
caaaagaaaatcacagaaaccaagcagttcgccaaagacctccgccagtggcagacaaatgtagatgt
ggcaaatgacttggccctgaaacttctccgggattattctgcagatgataccagaaaagtccacatga
taacagagaatatcaatgcctcttggagaagcattcataaaaggctgagtgagcgagaggctgctttg
gaagaaactcatagattactgcaacagttccccctggacctggaaaagtttcttgcctggcttacaga
agctgaaacaactgccaatgtcctacaggatgctacccgtaaggaaaggctcctagaagactccaagg
gagtaaaagagctgatgaaacaatggcaagacctccaaggtgaaattgaaqctcacacaqatgtttat
cacaacctggatgaaaacagccaaaaaatcctgagatccctggaaggttccgatgatgcagtcctgtt
acaaagacgtttggataacatgaacttcaagtggagtgaacttcggaaaaagtctctcaacattaggt
cccatttggaagccagttctgaccagtggaagcgtctgcacctttctctgcaggaacttctggtgtgg
ctacagctgaaagatgatgaattaagccggcaggcacctattggaggcgactttccagcagttcagaa
gcagaacgatgtacatagggccttcaagagggaattgaaaactaaagaacctgtaatcatgagtactc
ttgagactgtacgaatatttctgacagagcagcctttggaaggactagagaaactctaccaggagccc
agagagctgcctcctgaggagagagcccagaatgtcactcggcttctacgaaagcaggctgaggaggt
caatactgagtcggaaaaattgaacctgcactccgctgactggcagagaaaaatagatgagacccttg
aaagactccaggaacttcaagaggccacggatgagctggacctcaagctgcgccaagctgaggtgatc
aagggatcctggcagcccgtgggcgatctcctcattgactctctccaagatcacctcgagaaagtcaa
ggcacttcgaggagaaattgcgcctctqaaagagaacqtgagccacgtcaatgaccttgctcgccagc
ttaccactttgggcattcagctctcaccgtataacctcagcactctggaagacctgaacaccagatgg
aagcttctgcaggtggccgtcgaggaccgagtcaggcagctgcatgaagcccacagggactttggtcc
agcatctcagcactttctttccacgtctgtccagggtccctgggagagagccatctcgccaaacaaag
tgccctactatatcaaccacgagactcaaacaacttgctgggaccatcccaaaatgacagagctctac
cagtctttagctgacctgaataatgtcagattctcagcttataggactgccatgaaactccgaagact
gcagaaggccctttgcttggatctcttgagcctgtcagctgcatgtgatgccttggaccagcacaacc
tcaagcaaaatgaccagcccatggatatcctgcagattattaattgtttgaccactatttatgaccgc
ctggagcaagagcacaacaatttggtcaacgtccctctctgcgtggatatgtgtctgaactggctgct
gaatgtttatgatacgggacgaacagggaggatccgtgtcctgtcttttaaaactggcatcatttccc
tgtgtaaagcacatttcgaagacaagtacagataccttttcaagcaagtggcaagttcaacaggattt
tgtgaccagcgcaggctgggcctccttctgcatgattctatccaaattccaagacagttgggtgaagt
tgcatcctttgggggcagtaacattgagccaagtgtccggagctgcttccaatttgctaataataagc
cagagatcgaagcggccctcttcctagactggatgagactggaaccccagtccatggtgtggctgccc
gtcctgcacagagtcgctgctgcagaaactgccaagcatcaggccaaatgtaacatctgcaaagagtg
tccaatcattcgattcaggtacaggagtctaaagcactttaattatgacatctgccaaagctgctttt
tttctggtcgagttgcaaaaggccataaaatgcactatcccatggtggaatattgcactccgactaca
tcaggagaagatgttcgagactttcccaaggtactaaaaaacaaatttcgaaccaaaaggtattttgc
gaagcatccccgaatgggctacctgccagtgcagactgtcttagagggggacaacatggaaactcccg
ttactctgatcaacttctggccagtagattctgcgcctgcctcgtcccctcagctttcacacgatgat
actcattcacgcattgaacattatgctagcaggctagcagaaatggaaaacagcaatggatcttatct
aaatgatagcatctctcctaatgagagcatagatgatgaacatttcttaatccagcattactgccaaa
gtttgaaccaggactcccccctgagccagcctcgtagtcctgcccagatcttgatttccttagagagt
gaggaaagaggggagctagagagaatcctagcagatcttgaggaagaaaacaggaatctgcaagcaga
atatgaccgtctaaagcagcagcacgaacataaaggcctgtccccactgccgtcccctcctgaaatga
tgcccacctctccccagagtccccgggatgctgagctcattgctgaggccaagctactccgtcaacac
aaaggccgcctggaagccaggatgcaaatcctggaagaccacaataaacagctcgagtcacagttaca
caggctaaggcagctgctggagcaaccccaggcagaggccaaagtgaatcgcacaacggtgtcctctc
cttctacctctctacagaggtccgacagcagtcagcctatgctgctccgagtggttggcagtcaaact
tcggactccatgggtgaggaagatcttctcagtcctccccaggacacaagcacagggttagaggaggt
gatggagcaactcaacaactccttccctagttcaagaggaagaaatacccctggaaagccaatgagag
aggacacaatgtagtcgtttaaaccgctgatcagcctcgaaacttgtttattgcagcttataatggtt
acaaataaagcaatagcatcacaaatttcacaaataaagcatttttttcactgcattctagttgtggt
ttgtccaaactcatcaatgtatcttagtaagttttttaacaagcatgggacacacaaagcaagatgca
tgacaagtttcaataaaaacttaagttcatatatccccctcacatttataaaaataatgtgaaataat
tgtaaatgataacaattgtgctgagattttcagtccataatgttaccttttaataaatgaatgtaatt
ccattgaatagaagaaatac
SEQ ID NO: 156
Intron 44 sequence of human DMD
gtaagtctttgatttgttttttcgaaattgtatttatcttcagcacatctggactctttaacttctta
aagatcaggttctgaagggtgatggaaattacttttgactgttgttgtcatcattatattactagaaa
gaaaattatcataatgataatattagagcacggtgctatggactttttgtgtcaggatgagagagttt
gcctggacggagctggtttatctgataaactgcaaaatataattgaatctgtgacagagggaagcatc
gtaacagcaaggtgttttgtggctttggggcagtgtgtatttcggctttatgttggaacctttccaga
aggagaacttgtggcatacttagctaaaatgaagttgctagaaatatccatcatgataaaattacagt
tctgttttcctaaagacaattttgtagtgctgtagcaatatttctatatattctattgacaaaatgcc
ttctgaaatagtccagaggccaaaacaatgcagagttaattgttggtacttattgacattttatggtt
tatgttaatagggaaacagcatatggatgataaccagtgtgtagtttaatttcaacttgtggtgtcct
ttgaatatgcaggtaaagatagattagattgtccaggatataatttggttgctaaattacatagttta
ggcataagaaacactgtgtttattacacgaagacttaattatttttgcatcttttttagctcaaattg
ttcatgttgcaatagtcaatcaagtggatttgaattgtagccaatttttaatgccagaaaatactgat
taagacagatgagggcaaaaaacacccagtagtttattaaatactttagatatttcaaaatgctggat
tcacaaaagcagtatcacatttgactttacaagtcttcattctcaaatatgtttccatagtaaatatg
ccctttaatattaaggagttaagcatttaaacacctatttatatgataagctatttaaacacagaaaa
tatttttaaaaccttgtgtaattatatgtgtatcaatcaaacttgcatgcacaccagcgttggcattt
gtatagagaggaaatgtatggattcccaatctgctttaatatagaagatacattttaaaaatagcact
gaagtgaattttgggctaatgtagcataatggggtttctgcctgagaggcagaaacatattagagtta
tataaaatgttttggggtagatatagaaaccacttgccattttcaatgatatccaacccaaggtagtt
atatatttcaatttatattttattatcaaattagtacttattgtgaaaaaaatcaagtaacatagaaa
tttgtaaaagtacctccattctactctttggaggatagttgttcagtatgaattttgctacatatttc
aggctgggtttcttggaaagccattgtaaaatggagatttgtatgtagaaggttaactagggagtact
tttacgatgaagcaatttgttttgatgtaacttggtgtagttttcttcatgtttcttgttcttgaagt
cagttaagctcttgaatctgtgcatttaacatttcatcaaatttagaaacctttcaaccattttttta
aaaaaaatggaactccaattgtacatttattaggctccttaaagtgccccactactcactgatgttat
gttcattgtctgtttggtctctcttttctctgtaatttttttatataatctctattgtcaaattgac
taatctttttcaaagtctaatctatggctaatcccatgtagtatatatttttaacatcagacattttc
atctcttagaagtaaaagttgggtctttttatttcttccatgtgtctactcaacatgttcagtcttta
ctttcttgactatatggaatacagatataataactgttagaatattcttctctactaattttatcatc
tgtgtctattctgggttaatttaaattgatttatttttctcctcattaagtgtgttgtttaactgctt
ctttggatgactggtaatttttgactatatgccagacattgtgaattttaacttagcgcgtgcttgat
acttcaaataaattcaaatatattgaaataaatattctcaaacctcgttctggaacacagttaattca
cttggaaacaatttgatcttttgagaatcttccttttatgctttgttatgaccagaacagtgtaagtt
tagggctactttttccccactactgaggcaaaacccttctgagtactctctctgatgtcctgtgaatg
ataaaatttttcactggggctcgtgggaacaggtggtattactagccacgtgtgagctctggtgattg
tttcctttaattcttttgtgaagttctttccttagctttgagtggttttcttgcatacatgaactgat
caagactcagatgaagaataaaataaagctttctacaaatctccaaaatttcctctgtgtatatatca
cctctctggtattttgccctgtgatcactagtcagccttgggctgctgaaactctcagcttcatcttt
taacaaaagcctcctggcaaggatcactgtccttcaatgtctgatgttcaatgtgttgaaaaccgttg
tagcatatattttgtctttttttttttttttttttttttaagtgtttcaggtgtttcaggcaggagat
taagttcagcctcctttactccaacttgaaaacaagtccaaaacaaactattttgatgtaatttgatc
ttttaatacattaacattacacaattttgtgaatatatcataatttaaaattttcagagaatgtctaa
tggtcctcatttcttgacagtgtggtttagttgaaactgatgaacattttatcaaaacttttcccctc
aattggatacttttttttttttgagatggaattttgcttttgtcacccaggctggagtggcatgatct
cagctcactgcaacctctgcctccaggcttcaagcaattctcctgccttagcctcccgagtagctggg
attacaggtgcccacccccacacctggctaatttttgtatttttagtagagacgagatttcaccatgt
tggtcaggctggtctagatctccgacctcaggtggtctgcctgtctcagcctcccaaagtgctgggat
tgcagacgtgagccaccatgcctggccaactggataattttaaaaagaccattttatttagtctattt
tttctcaatctatagatgagataagaaaaatcattctagatgtccaaggaaaaattctttcagaaaag
agctgtgaatgatatcacaaaccccccaaacagttaaggtatttctttcctggttattttatgtccaa
aatcatgcatatgaacatgtgcacacacatgagcgtgcacacacacatgaatacatatacacgcacat
aatgtaccttaggttatctttccattctgagtaattatcgtaaaatgggtaaaatcaaccccgtaaga
taccttcatcgataaggcaaatcaaagctttggtaatttctgctatcttggcctttgttgattgacta
ataatgaataagagaatgagtttcaatatttactatgaaattattttagaagacaggatgtagacagt
ggctgttagcaggcaattgtttggcatgagccagtaatggttactgtgaaaaaaatcaaccaagcagc
ccatatattaaacaaacacacgcagaagcacgttggagtctgaagcctcatatgtacaattttcagta
aagaaataacttttagatatgaaataaacaaatagatatatgttgtaaacttgtccctatgtattttg
atcaaattgcatcatatttttttcactttaaagaagagaatttagtgctttaactgagacttagtgtt
atcattcaaaatatactgactgccaatagcagtagaaagataatctggttccatgcaactctattttt
tttcctctgtcgcaagtaaaagacaaaattaagtacatgaattagtgctttttgaagatattccagag
caatataccatgccactatggagaacctctctaaaaatatcccatttttttacctgagaaaaatattg
atcatgttatatgccactcaaattggtttattaaattcgttgaatgatatcagcatctcttaatgcat
tcactaaacaagcagtaattgagtgcatatacaaagttttatcatccaccaaaacagtgacaatccac
atgaggctctaatagaagtttagaaagggggttaagtggttaaatgctggactcagaaagattggatt
caaatcccaggtcctttagcttaatagttgtagaatcttgtgaaaatatcttaattcttttcatgtct
ctgatttctcttctctaaaatggaaatataaatgagatgtgtataaagccacttggaatagcattttg
cacaaaataattactcattaaatgtaagcccctattataactaatcactctttataagtgattagttc
atatcaatacaaactaagacttatttactgaattatcgtctctaaacatccacactgcagaaaaacca
acctggaaatttcataaaaccttatttttatgtagtataatttcttctcaaagcataagggctcttgg
attaggaattgaggaaaattccaattcagccaaacgcatctgtttcagatagctgacacttctgccta
ctcatttcctagctaacaagaagaaatgttaatgggagttttcaaaggaaaagctgaacaccatgaag
gaaagtgacacaaataatgttagctcatatattgacagggtgaatttgtgtgctttcaagtcccttca
gtgaaaataggaaagtagaaattataaaatgccctaacatttaaagctagcatgttcttggagactag
gaaaaaataagttttaaaacatgggctatgatagaatgagatggaaaatgtttgtagttgccagtaga
aacaataacaattaccattagattaagtatttaaaccagctgaatatttttattaatggaaatggcat
ctgttttatgaaataatgctgctgaatgaaccatattaaaaatgaccagtatttcctgcagaacgttg
tcgcagacatacaagcctgagaccctaaaatcttaaggtattccatttgaaatcgaccttaagacatt
aacagtagtggtattgtttagatgaaattttttaggctttaaatcaacaaatgttaagcagacatggg
gagcgaaacaccagtgtgttattctgacatgaataaactgctgtttttagggaaaaaatatagtcttg
ttaaggttaagctaattggttttctggtatcttttgcaatgttagtgtgttttactgctccataacct
atgttatatggtaaatgtgcaatatatttatatatgttgctgtaaagaaatgtaataaaaaactgttt
actttgtgatatgaaagtaaaaatttattcattgtcattgagcatacagaagtaaatatggattacat
atgtcatattttaatgttcacatggtcccaccatcaaatgttgaaaaacttatagtttaacgtcatat
tctattgaagaaaaatacactcccttttctcaaatgtgaaatgtccagagagaatggaaaattacata
taaagcatgtagttatagcatggtgaccctgctgtgatctctcagatgaggaacaaaagggagaaaga
aagagcacactggtgctttggagttgagagaaggcaaaaaaagagtacaaaaatgtcaaagccaagtt
tagctgctcttcagctctccctttagctgctcttcagctttaccttaccatggttattagtgattgaa
gaaaattctaaagcactttttaaaggacccaattctgaagagtttagattcagagagcacaatggagt
tggagtgactcctgctcaaaagtttgagacaagcgagtccatgaaaagaccgtcctcctcttaatgga
aatacccaggttttctcattcttctcgccttgctttcagcactcgcagcccagaaagcccttatctaa
caggtactgccgttgaaaggtcattgacttgtacaaaaatgatgagtgctgaatagatgtgcataggt
cactgacagtatctgctacagagaatgagttttcgtatttttattaggatacacctaacatggcaatc
tactgcctcaaagaactctataggaggtaagtgaatttatattaatacagattgaattaaaggataat
ctagaaaaaggcatatgatgtaaaaaaatcagacacaagtatattttctgtatagtcagtttttacat
tgtgatttcaccagctggctgctgagtttgacggcttcttaacagccacactgctgagattcaaatgc
tgatagaaactttgatggaaaaatcactggagtaaatatttctaccatctgttgcccttcactgggac
cctaacgttaagaataattcataccattgcttgtcctttatatttccccagcagtaataaaatttcat
aagattttgttttgtggtcacaaagctatcctggtttctgtaactagaagacatacactagcataagg
gaatcagccggaaaatttactgctaagagaatttgtctctagtcacttactttaaggttacagcaatg
tgtaagtgtgggaatacattttaaaatgagcttttcaaagttattagctggtagtggcatgagagtta
agtctcttaatacagttaaacagttgggcacttcatccttgcgtaaatattgttacccttttattgct
gcttggaaactcctctgcaactttttggcccctatccatcttttcagaagtagtaaataaccaattta
ctgggagtgtggtaccaggcagaaattccgagaggggctttcaatccttgcccatcaagtgtatcttt
cagaaataagtatattaaaataattggataatttcagtggcttgttattagacttccgttgtccagca
tggcatgtttaagaagatgacagattttcatacattattggaaagaagcaagaacaaaaaaacataac
ttactgtagtaaccacggtaaagaactgcttaaaatgcaggataaacatgtcatccctaagggattcc
cattcttagagcatgaaattatcaagagagtaagagactacaaaaaatgagaagaatgctgattgcaa
attccaaatagaaaaaatcaaaacaaaactgcgcaccatcattctggaagcaatgagaagcagaaatt
gtcatttaatgaaatgtaagattaaagttaatagaagtaattttcatgaaataatattttgcaaggac
gatgttccagccatattgatcttcgtgttttcttttcacatcccttcttactgttccctagaatgctt
gtttctacctttaaatttgcttttctctctaccagagggctctaccctatctccagtttctcaccatg
tcccaatctactccctctcagaatttttgtacacttccctttatatatatttgtgctctaattttata
ttcacagatatgccttttgtaactcccccatcttaaagaaagcacacacgtacgcacacatgcacaca
cacaaaattgaactctttctgggagatctgcttaactttcttcataactctgtcacttgctgaaactg
tagtatgtgttttcatgtttattatcttttccattagaatgaacatattttgggtacttggtctttct
cgatcaccaatatacctcggtacgtagaaaaattgattcatatattgaaaatgtaatattcagtagaa
cgaataaatacataaataaatttaaaaatgatacttttattgtattacctgagacaaatgatccccaa
gtttgtccttgcttttcatagccaaaacattctctcttacattgagcttccttcacctcttctgtgta
cagagcacttaaaattttcacattgcctgatactttaacaatatgatggccctgttctcttacccatt
ggagcatatgttaaataccagaacccatgtaacaaacatatattgtgatcctactgtgtgcaaagcag
atactgcttgctgctaggaatacagagctgactaagagctccttttctctttatgagctcacagtctc
atgagttcaacgtcttaaggcacaacgtctaaagcaaagggcagtaagtaaacactccagaaagtact
ggatctggcctaggacaaatggtgggttgtttttccagctgttatttttcctgccccctaattgacag
tcctccattacacctctgggatacctagtctgacttgggaaaacctgactttgggaatcagaggcagt
ctctcttgcttatatatgaggaactctaatggatacttactgtcattagagaaactctgcttctagcc
tggctccttttgtaaagaaggttgagtccccttggagagcctgcagaacataaccatttgcatgtaat
gaacagtttgtaatactttgagattgatgtgcaatttctatttgacaagggaaaaacaattaggatta
accgtggtcgtatatcccagaataccaacgttgtttccacactctaagtgttgttgggtcattatatg
agattcataattttgtcctgttgtacccacgtttgcattaccattcagtcttaatttattatacccta
ttaaaagtttttttggtaatttgttcttattgctactcaggcattaaaatgtctgcaggctgtgaaaa
tgaataaatttaatgtggcagcatagttctcaaaatcctggctttacaactcatagtacaggcttgta
ttgtaaatcctagttaacatggatttatttgaaaatccaattttactgctaatcttaaataacacatt
tttcaaacattttatccttgaatttctatttttttataatttatggctgttgtatgtatttacaaaag
gacaatgtgtgtacttttaaatactagtaatggattgctgaaacaactgtaactttaaaacaatgcaa
ttgttaaaaaaataaactgtgcagcctggcttaatggaggcttatgaacatatgattaagatatatgc
tataataagcaaattcactcaactgatagttcataggaactttcaaatttaatctcataaccagtgct
atccttcaaagaatggtcagggcaatttaacgagtacatgaccacgcaagataatttcattgaagagt
ggctgaactgttgaaatattttctagtctccttgggatatcattaagagcagaaattttgaaatggaa
ttgtaatgatgttcagaaaagataagtaggtaactctcttaatacgttttgtgctgctgtaacaaagt
acctaagactaggtaataatttgtaatgaacaaaaatgtattggctcacagttctggagactaggaag
tctaacattaaggtgtcagcctctggcgagggcctacttgatatgtcatcacatgatggacgattaga
gggcaagaaagatcaaaagggggctgaactcccacttttataagggaaccaaacccactcgtgagggt
ggagccctcaatccttaatcacctcctaaagctcccaccccttaatactgtcacaatggcaattaaat
ttcaacatcagttttggagggaaaaacattgaaaccatagtagtgatactgactactaccacacaggg
cttgggaggctaccctagctgttgcacccaagagatgaatcttctaatgtgattacctttatcatttt
ttttactttattaaaatacttttattttacatgtatacttttgtctacccaccatttccatgtctgac
cactgctactactatgtcctagcataacattccatacatccttaaaaccaagcaaagggtggagttcc
atctttaaaaactaaacaggcattttggacaacacattcttggcaatggaatctggacaacatttatc
aaacatggtagggaaggttctcactctgcattatcaaaacgacagccagatatcaactgttacagaaa
cgaaatcagatggaaaatttttaacaaattgtttaaactattttcttagagagacttcctccactgcc
agagatcttgaatagcctctggtcagtcatctggaagcaattcttcacataattcatgaacttggctt
ccactttaggaagagaaccacctttttctatacttgcttgcatttttgctttaatgtcttctacagaa
ctaggtcctttgggtgttttaggagtttttccttgttttgaaggattcttgtccttttgatcttggtg
ttgacggttttgagtcttttccattccgatttgacttttgtgcatttttggctggagtatctcatata
gatttcttcactggcgctttttcttcagtttcctcatcatcaaaatcatcatcatcatcaaaatcatc
atcttcatcagcagcaagttttacttttttctgtggaaccttgctaccacctccaggagcagatcgct
ttccagatatacttatgagtttcacatcctcctcctgttcgtcttctgactctgtatcttcctcccca
gctactaaatgctgtccactcacatgcactggccctgaaccacacttcaaccgtaagaccactgatgg
tgttatttcaaagccctcaagggaaaccatgggctgtacagacattttcaaagctgccagtgttactt
taattggactgcctttgtaactcattgcctctgcttcaacaatgtgcaatttatcctttgccccagcc
cctaaactgaccgttcttaaagataactgttgctcaatttcattattatccaccttaaagtgatcatc
tttgtcggcctttagttcacaaccaaaaagatagttttggggcctcagaggactcatgtccatcatcg
tccatcaggtggcaggacgcacttaggtgggagagaaggcagatgatgataaaggaccactgctcaag
agaacagctgtgcaggacagaatcacaccagggagattacctttatcttagaaaacctgaacatcttg
tgtactttgacacttctctacatttcacctaacctttaacatcaacacatttattcagaaaactttta
cttttggagctgctctgtgtcaggctctatgctaggtgctcaggatattgaaattgatacaatcctaa
cctattcacatataatccaaggtttgctgaaattgatggacatttaaacaattgaaacatttaagtgg
tataattagcaaatggacatttaagccataaaaatagcatctaatagatataatagaggtcggtacac
cattgatgagtcagagcagaggcaacccaaagagtaactagccagaagaattgggaaagcttcataga
gagagcgatatgaaaataagggagagaattgtaaatccatgaaaatgagaaaaagttgaaaagtgatg
gtgtcagaaaaacttgtggtatgataatgacaagatgagaggaactcttggtaagcgtgttggatgca
tggaaagaaatggcacaaaataatgctgaggacattttttattttattgttggttttgttttggttaa
tttcattttttaaatctagtatgctagtgttcattgtccaaactgtgaatcataaactcagtttgtgg
atcaacaccggcctttgatttttagtgaaacaaaatagaaaatatcagcattcatcacaaatagatgt
ttcacagattttttgttttaattgcgactgtgtgtgtgtgggtgtgtgtgtgtgtgtgtgtgtgtgtg
tgtgtatgtgagagagagagagagagagagagagagagatggcttggatgtttatcacctccgaatct
tatattgaaatgtgatttccaatgttggaggcagggcctggtaggtgtgattggatcatgtgggtgga
tccttcatgaatgatccctttggtgacaagttagttcatgctatatgtggttgtttaaaagagtatga
gacctcaacccccacctgtttcctgctctcccctttgccttccaccatggttggttgtaaacttcctg
aggctctcaccagaagtagatgccagtgacatgcttcctgtacagcctgcagaaccgtaagtcaaaag
aaaaccccttttctttttaaagcacccagtttcaggtatttctttatagcaatgcaagaagggactaa
cacagttgtatgtgtatgtgtgtgttgggtgatttctggttgagtgtcacaaggttgtaatatggtga
gtgtaaggaagtataagttttagaaaattaagaagccagttcagaaaactaatacttttggaaaatag
tacaaaatcaactttacaagaatatacacagaaagatgtaatacaagatttatttcattgcagtaatt
tataaagttggtttagtgccttgcttttgcatgctgttttaaaaattaccaagaatatgacttcatgt
gattttgaaatactcccagcaagataggtagaaaaggtattcttataactcttagacaaaaatttcgg
aaagtttaaacgctttatcccaaatcataaagctaataaatgaagaatctgggattcaaacaccatat
tttttttactgttcatcagctagaagttagaaatgttaagccaaaaacattaagtcactgctctgcct
aataaatcttgaggaaactaataaaaagaataataccactgactacaggacaaggtcttcctaagaga
ccttaaatatattaagtgatgaagatgaaacttcttttattcataaaaatgttatttagttatgagta
gagctctaattaaacttattttatattgtcatcagtaaagttgagacataacatatttattaatataa
ttataatttgacccatagtgtattaaaagaaggatgttaaaaggagttgttattagagatgatgttag
ggttgttgatgataataacagtagtcataacataacaaagcacttcataatttaagaagtgccttcaa
ttacattgttactctcatggtaatctctgtttgatatatagatttggcggattctatatcactctaag
acataggttactgaggtgacggaggaatttagcaagcggctgtcaaatggaggacatgagcattggat
tgtgtatggcaagggctgatggtctctaagaaagcctcttggtttccacagggcagaagccctttgaa
gatcatagccaaggatttagtaattgcctccctttcagaataccctcaagagaaaagcccaccataag
acatggttccctacaggcaaaactgcttttccttaaaatttactgttccctgaatatcagccttcttt
ggctcattcaacatagttttcttaagtttcaggacagtgctgcagaccaaaagtttcaacattgagga
aaacaatactacttgtgcagtgaccctacctcagtcagggaggcagatgcctgcctttatgtgaggga
ataaggaatcaatcatatttccagcactcaagaaagccagtctagtgcagggagagatagatacataa
acctcaaagttatgatatagcataatagttttaaatttccataataactgtattttaaaagttttata
gaaacagaagagatgacctcagtctggaaaagccagcttggagaatggcaaccaatattaagtggcaa
aagctttgggatcccaggcctccagatggagggtgatagcatgggccagacaggtaggttaggaaaac
tttgcaaaggacattacacggtacacagacaagtctgtgttttagcctataaaccacagttgcagaat
gtgtttgagcaaaggcttttggggatgagatttgcacttttcaagatttaagtttgtttaggatactt
acggtttgctgtatacttcctgggtttttacattataattacggtttgaactttaaaggaaaactgca
gtttagcatacttgaaagagtgcaacttcaagtcatgattggagacagatatttaacagattttgtga
tcctgtgatgcttattttcttctcagacataccacatgacaatcatttttaaacagtttatttctact
ttagcatccatctgaaggtgttgtgtatgttttctgcttgaaaataaagcagtgggctgggtgcggtg
gctcacgcctgtaatcccagcactttgggaggccgaggcaggcagatcactaggtcaagaaatcgaga
ccatcctggccaacatggcgaaaccccatctctactaaaaatatgaaaattagccaggcgtggtggtg
catgcctagagtcccagctacttgggaggctgaggcaggagtatcgcttgaacccgagaggcggaggt
cgcagtgagccaagatcgtgccactgcactccagcctggcgacagagtgagactctgtctcaaaagaa
ataaaaaagaaaataaagcagtgaatgcgattaagatggatttattatgatcataaagtactcaggag
tcttattttaaaagacagcattactgtaattaaaaatatagggaagaaactaatgctgttttgcgtat
cattctcagctctctcaaaatcagatattaagctcttgctgccaaaggagactatactgcacggtgct
cacctgcataaactttgagagggttgaattgtgccaagcaattctctcaatacataaattaaccaaat
atttgttgacctactgtgtgacaagtattattccaggaaataagagatccagcaatgaaacaagtatg
gcttcttatagagttcccaaaaaggaaataaaaggatatacgtatagtgatatccctgaattaaattt
ctcttttgaaaataaaaattctatcataagctgtaactgccaacacttcaatactcattcagcagttt
tcagggatttgtaccttttgacttatgagaatttggaagtctaattgtatcattgcactggagtctta
aagaaacagataagcgaatgactttgcctgtatcattgttgactgtacttacaatcagaaaggggcac
aggacagatgccagggagtaagtggacagcccataaatggaatggtaagaaagaagaactatagtgga
tttggaaagttcccttcagcattttccctagacaatctttggctgtgtttgcatgatcagtatttcat
tcacaggatattgagctcttgatatagttctcaaaacccaaaatgaaataagaagtctactctttatt
taaattcaaattccagagagttaagtaactttccaggaggtaatctaaatatggcctccttgttgggg
ggggggggggtgtttgaatttgcatataaatagtctcacccttaaaggaaaaccacagatggtggtaa
tgatgtagtcataatgtacatctccacagtggtggaacaaaatatccacagttttgctttccccagtt
tcagtgacccatggtcaactgctgtctgaaaataggtgactacaatacaataagatattttaagagag
agaaagaaagatcacattcacatgattttcattacaatgtattgttataattgttctatttttattca
tgatttttaatctcttaactgcgccaaatttataaattaaaatttatcacaagtacatatagtttata
tagggctcagtactatctgcagtttcagacatccactgggagtcttggaatgtatcccctacagataa
ggggtaaaccactgtatcctatttgtgtgaatgctacaggtgttgtgagctcataacaatatgacatc
aacactgaactaatccaggatttggtagtgagagtgatgtatttgcaaggagtgagacgtggtgcctc
atccaagcagagaaataattttgaaatttgcctgacaataaaaatcacaatgtgaggtctctctttag
agctgcaaagtccaattcagtgccccctagccacataagatactgagctcttaaaatgcggctagtac
taattgagatgggcactgagtataacacacatgccagggtttgaatacttagaaccaaaaaggaagta
aatgctcatttattgcatgttaaaattatggttttattatagttgattaaataaaatatataattaaa
ttgacttcattttgcttttaaaaatgtggctatgaaaaatttcaaattatatatgtgtgtgattacat
atgtgtgttttcacatatgtaactgatgttacatgtgaaattgattgttacatgtgacatgtaaaaca
cgttacctaacacgtgcatatgtatgcaacacatatgtaacgtgttacatatataacacgttacatat
gtattgttacatgtgtgcttgcattacacacatgcataatatgaaattacatgtaatttcaaattaca
tgtgtatattttgaaaattacaaattacgtattttgttatttttgctttacaaagtcaaatttaccct
atttaataaagcatcatgagttttttataactagtaaactttgagacttttgtaggagaataaataat
gcttattataaaaactgattggaaaagtgagctggagcagggagcggaggaaaaaggactagagatca
cctttcttcccagctccgctcctctcccaaccttttttctttccattctctcatcccaattcaaaagt
gcagagttcacagttggtgtgctgatttagaaaacagatatataaacagccttaaattttctccaggc
ttttacaatgaaaagaagttcaatatcaaaagtaacaatataatctgtggaaaggtatagggggctat
gtttttgaggtagaaactataggtgctcctggccaagcatggtggttcaagcctgtaatcccagcact
ttgggaagctggggcgagagtattgcttgagcccagaagtttgagtctagcctggcctacagggtgaa
actccacctctactaaaaatacacacacacacacacacacacacacacacacacacacacacacacaa
aagccttgcgtggtggcgcttgctgatagtcccagctactcaggaggctgaggcgggaagattgcttg
aacctgggagacagaggttgcagtgagctgagatagcaccactgcactccgacctgggtgacagagta
agactgtctcaaaaaaaaaagaaaagaaagaaagtataggcactccttatatgcagctgctcacaccc
ctcctccttcacacccctcccccttcacacccctcccccttccccaaaatttgcaaggggaaaaatgt
gtgtaattggcagtatttagtggcgtgcaaccgtgagtcatcagactgcacatcctcacttctgctag
tggctcagtacccaacagcactcagtgaaaactaactcatttcaaaggtgaaaacaagtgagtttggc
caccagggagtgttcaaaactgtcagtgctgaagcaaatgtggagggtgttctgtagtttgttcaggt
tgatatttgtggtccaacccctagctgaactactaattattaatatctgtcttgatggtgcctcagga
gaaagcttctcaaagggaatcaatgttcaaattatagtaggtatcttggccatggaagttattgaatt
ttagccaatacttgctactctttcatttatagtgtgagaatgcagtgtaatgaacctgactctcactg
tcctgacttgcctttctcatcgcattcacaataagcacgtcaatacgtatacacatttcatatttcta
aagtttactttatttccttattgtacatcgctgtgctgctgatggaagagaaaaggaaaaacactatt
gattgcaaaactgttttatctttggtggcttagattttttttgtatgatatgtaacgtcttgcatacc
taaggcaacacgaagctaaatagatttgcatatagcatgtattttttccaattaaatgtttaattttg
ttcagagtatactggggacattttgaataatggagaaaagtacaaagaaaattcataattctaccacc
tatcagcacagtgaaattttatgaagaaacataattttcatgtaaatcatagtgaactcacggtaggt
tttatttaatacagtaattggagagctggtaggaagacaaaactggttcaaaagagaatacaagaaac
aaatgcttctataatgagtgaatttttaaaaaagtattctggaataagattagtgaataagatactaa
actcgttgataccctacagcctttggggttatatcctctactgggtaaaaagtcatttacatcatatc
agttttctaaaatttgcattgaacttcatagcgttgtaacatgtgtgggcccaaattaatagtaaaca
gtaagagttgctttactctgaaaatattgaagctcttgtgagggtgtgaggagtttgttagaaaacaa
cgctaccattattttgaaacacacacgatcatcttttgttttacttctaagttttggataatttttct
taaattatcttattatcttatccattttcttaatttccttaaccttttaaatgtttctcctaggcact
tttattgatttttggaatatagttgatatgtgctgaatttttatcatccagttttaattctactgaaa
aatctaaaagatgttcatcaactactatatttcaaatgcatacatcccctttcatgctaaagaaactg
tatgggaaacacagtctgacattttcaggacctggtatcattaaaagtcttgacactgttaaaattaa
acaacgccttttttaaaatcaaaggatacaaaagggctgtgttggtcagaggatacaaaatttcagtt
agataggagacataagttcatgagatcttttgtacgacatagtgactataattaataataatatgttt
tcgaaaattactaagagagtcgattttaagtgttctcaccgcaaaaaaatagtatgtgaggtaatgca
tatgttaattagctcattttagctagtccacatttttcaatacaatgtgttgtataatacgtgatata
tacaacttatattttccaattccaataagtaaaaataaatgtaaattatttgaaataaataaaatgtg
aagaacatccacttttcatatgaaaccatgagatattttctgttaaaagattaaatgtccaataaatt
tttgatgttaacagaaacaaaaatgtttaatatttaaatacatatttgcatgctattgaccccctgaa
gttcactgctgggctaagtgaaccaactatatcttaagtcaaaaatgctgaaattcttccccaaatcc
caaagctcatgaaaacataaacagaaaatttccaaataattctacagggaaaataagacacactattt
gatctgatcaaacaacgggatgattatggttaataatgagttacttgtacatttaaaaataactaaag
gagtgtgattggattgtttgtaacacaaaggagaaatgcttgaagggatggataccccgttctccatg
atgtgattattacccattgcctgcctgtgtcaaaacatctcatgtaccctacaaatatatactcctac
gatgtacccacaaaaattaaaataaaaaagagagggacccgaagataagctaatatttaagctcatca
tacttattaagataagcaatacataccgaaagtaatagcatttaaaaccagatgttgggggagggttc
taacttcttcattaaaattcaaagtcacctgtcttgttttttcttttgtttttgtttttttttttttt
tttttgagatggagtctcgctctgtcaccccaggctggagtacagtggcgcgatcttggctcactgca
agctctgcctcccgggtttacgccattctcctgcctcagcctcccgagtagctggtactacaggcgct
ggctaccacgccccgctaatttttttgtatttttagtagagacggggtttcaccgtgttagccaggat
ggtctcgatctcctgacctcgtgatctgcccaccttggcctcccaaagtgctgggattacaggcgtga
gccaccgtgccaggccacctgtcttgttttatcatgatcccgagagtatatatgtatgtgtacagctc
atctaaaccctttttctttcaacatgatcaatagattgaacattggagatattttataagaaataatg
aagacaactcaatcagcacatatatatattaaatgtggaatctataatgattgcgaagcctgaagcaa
actaaatattcagtaataggttctttttttccatggtatatccatttgaatatataacataaatgcct
tacatttgttttaactatttaaggtttatgttgttagtgtgatgaaatggctggcaaaagtcagaaac
tcaggaaagtttcaggcttatatctggagcctggttttctttcttcaaggtagaacctctgtgaagtg
aaaaattttttttatatctggagcaataatgtagaagcttaaatgtattatccaagttgtcataagcc
tattatttctttacattactgaagtgaaagacagcattaatggctaaatgccatacttggctataatt
tatattgtttaggactggaaatgagcctgaaatgtacatttttttccaaaatagttcatgtaatattt
gaaacctgacaagtaacctgatgatttcatggaataccatcaaatataaatgtgaagttttaaagaca
cagggaaatactcagaataaaccccctaaccacaggccagcagaagaactagacttgagaaaatgaat
gggaagatagatagtaacaaatgacttctttggcagccttatatatgcttagtcttatagactgtttt
atggatgctctgcactctatttccagcaagtatggcatttggaacaggaccacacgagacaaactatg
agttcacatttcccacaactgcacagatagaaagagggaacaacagaatactccctttcttcttgaaa
caataacttctgttgaagctcactggcttcttttcagctgtttctgctagctcctcctccgcctcttg
acctctaaggcaatgctcttcaaaatttcaagactgctttctaattgaaacaaaacttataagcacat
ttcttcccacaaaatgtacatttatttgtaaatcatatatgaatatgactaagcatgtaaacgtatgt
gaaaatagaaatcaataaatataaatgcaaacacaaatagaagcattcacagttttcttttgtgtccc
agtgagttgttccaaattcctcggaggtaggtatgtcacagtttgagactataccttcaatcctaggg
tttctggtttcgctctcctcctaggtgatagcatccatttctacggacttaactgccatctttagttg
aataactcctctatctttccatcccatatttctcttgattccaaacctgcttgttcacctgagcatat
gacacaattcattggctgccgcacatgcagctttgacattttatttaaaatctttccccttccccagc
cctcatctatttcacagtagtatcttcttcttatctacttgattggtaagcagagtccacatgattcc
atcatttatctcccattttatatctaatctataagcaagtaatgcaatgcaacttctgtctccaaaaa
tttattttgaatttgccttctcttcctctgcatctcccccatcttaggccaggtcacctctgccctct
tgccagattaggtcacattctcttactactgttgttattctcttcctattcaatcctacaccgcagca
aaatggatcttctcaaaatgtcagctagataaaggcatttctgtgcttaaggccctcatggatttatc
ttattaggatgaacacccaactctttattatggcttagaatacaatgaattacaacacataatgaata
tattatatttctatctttaccattttcttcttaagtcaacctttctcaatccatataggataatcata
ttagtgcttcctcactttctaaaacatctcagggcctttgcacgtgtttctctgttcttagacccaga
atgctcttccttttctctttgtgtagctaggtgcttctttccatttacgtatcacatgaaatgcagtc
attccctcctccttccctcactacctcacaaaaagttgatgcctctgttaaaccatgaatggaatttt
actcggcagtgaatagaggaaaaaccaatggtaaaagcaaccatatgaatgaatgaatgtcaaaaata
ttatgctgagccaaaagtcatagacacaaatatgggtatttacatgaagttaaagcacagcaaaactc
aattacggtaatagaattaagaaagtggttacctctgggtgagggttggaattgagtggacagaggca
ttagtgactttttcggggtaatggaaatgttgtctattttgttcaggtggtgaatacatagatacatt
caattgtcaaaacacatccatccaaacacttagacttttgcactttattatatgcaaattatgcctca
actgaaaaaagtttgttttcaaaattatatcaacagttgaaattcttttaaagatttgattcaaatga
gattaattctgtatccatcattgatgtatgatagttttgtatgtagttaaggttattggagataattg
aaagttatactcacaagaaggctgcataatatgaagtttatctgccttgatctttaatagctttcgcg
atttcaacttcttcacagctctgtaagaaggcagtgtggcatgttgaagcaagcatgtgttttagagt
aacacagagctggtatacaaccccatgtctaccaattatcaatgatgtgggtatgttgctggatctca
ataatcttccactgtgaaatggaatgtaacacctgactcacaacgcaaaggtatttaccttatgtaat
ataattcctgcgatcctgggacctcccttaatcccatccacagatgccaggttaaagaccccatcaca
gactagaacaagttgggatgtcaaaatgaataaatattaatcgaagggcctattgtgattgaacacca
cgcagtaggcactctctaatacctaccgtctccctcctttttgggggaaacattctaaatgtgcaaaa
aataaagggttatttgctttctggcacttgggatcgatttattgaggatatgttagcagaacagcaaa
ggtgaaacactaaaagcaccatcaatacacaggcagaggtgaagccataaagcctttattttttaaat
taatgcacaatatataagaggtatgttagaatgaacgtccaatccctgaaaggatatacgaaagacat
tcataaaattacatgggcatgttttcttaatgttcaaaatattgttttaattagtgtattatgagttt
attcatgtgtctgtgtgttgtgttatattaatcttttcttgcattgctataaagaaatacctgagact
gggtaatggatgagaaaagacacttacttggctcacagttctgcaggctgtaccggaagcatagcagc
atctccttctgtggaggcttcgggaagcttccagtcgtggcagaaggcagaacgggagcaggcacttc
acctggctagagcaggagcaagagagacagaatgaagtaccacacacgtgtaaacagccagatctcag
agaactcactcatcatcatgaggatggcaccaagaggatggtgttaaaccattcatgagaaatccaca
cacatgatccagtcacctcccaccaggccccacctccaacactgggaattacatttcaagatgagatt
tgggcggggacacatatccaaatgatatccatgtttaatcagaaaaataaaagttaacagtaacagtg
attttactttgtagacctttgctaatggctgaaatctagctccattccgagaacagcctgcggtacac
attttgaaagatagttgattaatatgaaagaagccttatctgtagtccttaaggccattatggtttac
atatatgagtaaatattccaaagtagccatgccagttaacatatatccagagtctaaaggccactggg
cgacaaaagtaaaagatacatagcaattgttactttatatcacagtaattcttgtatattttaaatgg
atatttgcatttgaggatatccacttaagagttaggtacatggctcttacatttaagtaacatttact
taaatttctggctgcagcaattccacataggtagaaatgaagtctgaattgagttgggggtctttgca
gtgctctctctgttcattggctattttgacaatgctgagagatgtggttagccattctttttcatttc
atattggcaacctagagagcaattaagccttctccccttaactagatgtatgttttactcatttctgg
atctttatggctgactttgaatcctagcctgtggtagaaagcatggtgtcagaaggaactatgagtta
agactatgcatacttggctttgagtcttgggtatcatacctccctcatagagtgaaggaaccagggat
tcttcttgaggcccagacccggcatccatgttaagaatacctgtgcaattttgcttcctgatatttaa
ggtgaaaatgcatgtttgggtcattgtgaggattatgtgagatgttacttttaaatataggccccctt
attatatgctctcatagtttcaggcaacacttgtcgtatttgtaacctcagttttaactgtaatgttt
ccatcaatgtccctcttacctggtacaggggctcttcatattcttggattacaaatctgtgaatgcaa
ccatgcatcaaaaatattcagaaaaacaatgaatgcctacctctgtactgatgatttataggtgtttt
tcttgtcattattccctaaacagtacaatgtaataagtatttatatagcatttacattgtattaagta
ttataagtaatctagagatgttttaaagtatataggaggatgtgtgtaggttgtatggaaatagtatg
tcattttatatgtcacttgaacatttgtggatttgctatccgtggggatcctggaaccaatcccccat
ggatactgagggacaattgtattataagcagcaagagggaaaggaatctgtctattttgcccaaaatc
gtgttcccgggacctagcatagctcctggcaaagagtatacaacaaatatgcattgaggagagaacag
agggaaccattatccccttattctcgctgttccttcatgtaatgaataaacagtcaaatcttacaaga
gattttaaaccagtcagagaaaagttggaagttagttagttgttcatacattgagaagcctcgacgct
gtgtcatctaggtaatgaaagatctagggaagtttagcagggagaagaagagagatgatagttgtctt
caaatgtttgaaggactgttacggacacaaaaatttaaacttgtgctgaataattccaagaggtacac
agtctctcgatagaagctaaagtggggggtgacatttgactcaacaaaaagccatctaaatatcagaa
ctttcaaaagcaggaactggtgcctcaattaatagtgtgttttctagcacttatgatacctgatcata
ggcaagataatgaaaaattgggacctgggagttatacatgggaatttgtttatcagttgggtgattag
gagaggtggccttaaagtcctgttgtgttctaagagtctgtgattctgagtcttatttcccaacaaga
gaggtacagagcagaagatgggattgggagaaataggataaagataccaggaaatcctaaaggtaaga
aaaggaaggcagacctgaagctaactctatacttcaggtgcttgcctagagccagccctacctactta
gagaatgttgaagagccagttaaaacatctttaacacggatgtaaaacaaaactatcaaaacctgaag
atttcgaatgttctaacctactcgtcagttgggcttttttcacaaatacttcagtaaataggcataaa
tttattttttaatgatagaaaatatctcttaaagaacttataactgtggataaaagcaccaccataaa
aatcttgtggtgaaatatatatatatatatatatatatatatatatataaaattttaaatatggttag
ctagaatatgacgacaatgtttatgaaacacagagactcttgacaagtcccatgtatacactataaaa
ctttaagttatccactattcactcactaagcttatacttaatgagtgtctgctgtgtcacttattgcg
gaaggcacaggcggtatagcattgcacaaaacatatgtggtctctgatggagtttttcagtctagtgg
tgaaagcagtgaatgggtgtacagatgttaaataattgtacaattagttgcatgtgtaaacgtcaaag
ttcagaagatgacaattgatctacggcaatgtttctcaatctctgacgttttgagccaaatacatctt
tgttgtggtggactgccctgtccactataggatgtttggcatcacaactgacctctgcccattagatg
ccaatagtactctcttctttaatcacaaatttgtcccagacatttccaaatgtcccttggggagcaaa
atcatccctagttgaaaatcactggtctagggggaggtctttatgaggaagtaacatctaagaaagct
ggtatgtttacatatagctacagtctattacacatgtatacatatgtaacaagcctgcatgttgtgca
catgtaccctagaacttaaagtataataaaaaaaatgtaacaaaacaatacagtatgataagtgctat
gggaccaaagatgaaagggttctactgcacagttatgaactcatagttaggcttttggggtcaaaatt
ttgctgaagatatttgccacccacgtgacctttggcaggtgacttagcttattcatgcctcagtttta
tccaatgtgaaatggggctggaaagtcccatgtacttcctaataactttgcggaaataatatgtggtt
atataggaaaaaaaaaaaaatcctagaagtatgcctgctgcgtagtaaaaggaaggagaaggataaag
agaaatctgcattttttcttctgtaatggggcagatagtaaatattttaagttttgtggcccaaatag
tctctctcacatttacttgattctgcagttgtggcattggaagcagctatggacaatacttaaattag
taggtgtgcctgtgctttcaataaaattttataaatacaaagtttgcaaaacaaagttgttttttttt
tttttgtagtttgctgacaccctagtaaagaagcaccattgtcaacgttaaaaattatcaaattttta
tttttcaaagttttcaaatttgctttgcttggtctagctcatgaaataagtcaaaagtagcaagacct
ccacctctaaaataataatagtaatgataacctcaaaaggaaagaagaaatatttttaaagaagaaaa
attattgttaaataggattattgtgcagagaaaacctaggagactcaattttaaaatctgtgaaataa
ttttaaaaatactttatgaatagatacataatagcttttattcatattaatgactataaatgcaaatg
gaaatatttcattcacactgatgacaatgtataaattaaggaggaataaaaattgtagaccctatagg
tgaaaagcataaaaatatacataagaaaaagcaaaaattgactacgtaggattgttttaggatttaag
atttattgtcattaaacttgcaataccagccaagttaacatttgaatttaatacagttataatcagaa
tgcttttgatgtgtttgggggcaatataatttcaaaggaaataggcaatgatgtaatttaaagtttat
atagaaggaaattgtgtgcgtgtatgtgtgtgtataaattggaaacaattttattaataagcatatta
tggcagcaacatacacttccagatttctactatactttgaagtaattgtgatcaaaaccacagtgtgc
tggcataaggctagagaaatgggttagtggtttacaagtgagagtccaggaaaacatccaaataagat
tggatattttagttctgtgtggatagcctatttcacttaataaatagtgtctcgtaattgactattca
tgtacctataagtttaactatagaccaaaaaaacgccctactagattaaggagctaactagaaatata
aattcatataaacaataaaggaaagtgtaggactttataagcttcatgggagacagatttttggtaag
tcaggaagcctggaagacttaaaacataaaattggcagactgaattaactgatagtttaaagcttcca
tagagcaaaataaatcataaaccaagttttaaaatatataatggatttagagaaggtatttacaaaaa
tatatgactaatggaggttaataataacaatatgtaagaaggatatgaaatggcattttactataaag
gtcaaacaaatgacctataagcataataaatcatattaatctccactagtaataactacacacatcta
cataatatagatgttacgcctgcatttgatttactttatctgtcttttggcagaactatttgtcacca
gataaaaaattctatatcattaccagaaaggtatattattataatgtttattatgttgcagttgtaaa
agaaataacagcttttcaattgtttacaaatcctatagaacatttactgaaatacatttacattttgt
ggcaaacttggatttaaataccgtgttcgtgctttgttttatgccgttttcccatcttttctccagga
atttgattgtgcttcattgaaagctaaaaagaaaaaaaaaataattctggttttggtttaaaaaatta
ggttaggggttaaaaagttgtacgttgtcttctgtaaaaataaaaaacaagttttcttttgttcttgg
aggctttatattaaatggatttttaattcatagacagcatattgtgatgaaatttccccatgagcttc
acattttgtttcaatagcagaaactaacttggttgcagttactgcccttctgagaacagtgttctgga
ataattttgacatacatatgtatctctttttaaaacatgtgttaatcttttcataaagaaagttttcc
cagctgtgtcacctgtgactccaactttctggggggacagggatatgagatgttggaagggaatggct
tgaagaaataaagtgcaaaagacgtaatgctttcctgtggtagaaatgtattcagtgaccctgaatga
ccttcctactcttgtcccttcatttttcccacaagtatggtctgggcaattataaaaattgacatttg
cagtgggctcttctgtaaaagatgctcaatcagaaatgatttattttagaaaaagagatgatataaac
atatatatcccctgtctcggaagtgtgaaggttgaaaagcaaggagatgatcttcaaagtgtctaaaa
tattgatttgtaacatcgttttatgaaagtgcttcagattattttttttcttggatggccccttatgc
tttggtcagttgatgctaaaatctgaacttctttattttaaaaaaaacttttaattttgaaaaaggaa
gttcacggtgctgtctaattctttttagatagtcattaatgtaaatgtaagagtcattctgagaacca
catctgctgatatgttccgttaaattacaagttctatgtgtatttgctttgctttcatacaatgaatc
ttctttactctcttccccacctgccagaaattgccccactcaacgttcataaaaggtccattttcaat
cgctatatttatttcagaagcagagatatcatatattcaaattttagttactttccaatatcaagcta
ataactcacacaaataaatcaaactacagcaaaacagcaatctagcattcaacaaaacctccccaatg
cacatatttcaagctgtagatatgtatcatccaccatgctgaaataatgtacatgttcaaatcaaatg
gaaaactagaatcaaaattgttgattacttcttatcagggcattttattatatttaagaaaaatacaa
attaaatcattttcaggaagcaatccttctggctaagatttttttagcataatgcttaaagttaattg
ttgatctttatctataaattcaaaggtggactaaaaatgcagaatcaatcaggtagtccattttgcat
caggtgaaatatataaagcataaaacagcgagttacatttcctaacaaaattgaattacagtgagtaa
aagtgacaggacaaatgcattaagaaaagatggactgaaatggatagagtagaatatatgcatctata
aaacacagtcatatataatacactcattttttttcttacgagtgtgagattaatggaagaaaacaaca
ataataacaaaaccagtgtgatgtgtcagatttcaccttttaattaaaaaattattcacttcagaggg
gaattttctttcttgggttagctcaatcatgtcagatcttgttcatttaaaaggtcagtttacttgcc
ttctgaggtttttgtttgggaaaagaaaaagaaaatagattttcattggtatcctgggtagaattaat
tgtttatcattcatttttaagatctccgagaggcagaaaaaggggaactgtgcaacccttttgtcctt
ctggatctcaaaatgaagggatacattctgctacatgaaatgtggaattaagaccatgatgcaacatg
ataaacaacacaaatttgggggtgtctctgtgctatacattattgaatttttccatgctatacacttt
ttggatgtgtctgtgctatttattcagtttttttaaataaaagtttttgtagactaaattgccctctc
tactttgcatcgtttttgaacaaaggattttcaagactgataagctcaaatgtatcatttattgtatt
caagtagcattcaatttttctttagaagtataatttgtagatattttaacacagaaaacttgcaacac
tgctcatgataggcacttattatatattttttgaaagactatatggataatgattctaactttgactt
ttcctgttttgccttcactttagaattaagcagagaatcaaatccatattcctgggggcgatgcttgg
acaacagtatctctttaaagatctttgtgtgagtcgaaggtgcagccagactgggagttattgtgaag
aaacagattcaggaaggttgagaaacttgcctaaggctaatcagatagttactggcaatgttgtttct
aaatcactgtttggctccctcattcaatgaatctacactatgtgggactgcctcttgctcctgacatc
ttttgctgctgaaataaatgaactcaaagcctagaaggtagaaaagagggagttcagaattatattca
ggcacaaataccaataaggctattgcccccagaactgcaacttctcttggtttaacagataactattt
agctgtgaggtacaactgaggaagtggacacacaagttatcaggagattctgatgtgccagtttatat
ttcttgtcacaggtaatgattcgaaatttcttaaaacagctgtcctcacagtggagtaacctgggagt
acatgaaggcattccaaggagtaggcacagatagttttaagggaatttatttctagatcttctacttt
attttgtactcttcctgaaaactgaattgcctgaaaaaaaaaaaaaaaaaaaaaagacatctgtagtc
aagacctcaggctgtttctcctttctaaccacttgccttttctaaccacttctcccaatttaagaaaa
aaagccttatatttcatccaactctgatcttactaaggcttcaaacaaaagaagcatgaatgactttc
atgacagggcaacatagctttttgcaagaagagtggttgctaactctttgctttcaactgaacccgaa
gagaagacctgataagttgtcagccgatagatcattaaaaatacgttttggtaagcaatcatcatgta
cttttagcatatgccatagcaggagcacaaatgattaagcaatgctactataatacaattccttccgt
ttctttctactcacctatttgaataagatttttcatcatttacatctatacagacaaaaattagggat
agaattgatgctgaagcctttccaattgtagaattaatttatattcttctgaaggtgtataaattgtt
aaatacccatccatcttattaagagatgtattttcaataaaattttatttttatgtttatcaaatttt
ataatatacatatattgttttggtcaattgcacgttaataattgtaacaatacctcaattgaaaaggt
ttgttttttacatttaggacttacagtaacagaaaaaaaacactcattgtgtatacatactgtttaag
aaaagtatactaggtgatcaataagattttttcaggcataaacatatatcttagttttaagatatcga
tatttacaatctccctcaaattatattattttcagtcatttaagaatgaaaagtacatttcgaatgcg
gattttaaatctgcaagggttgactcatttttcaagagtctttttaggggatacagaagcaagaatgt
ttggagttccctgatcagtatctttaagagaaggtatttgttggtagttcctagcaaattccaacagc
ctgatgctacttaaaagataatagtaattattttaaataatgcttctgataaaaaacattcatgcaca
ctcagtttaaaaagatatttaaacatttgtagttgtagtttgggaactcatgatacaagtacagtctg
taaatgaagctcttagtttgcaaatatcagagataagctattaaaatgcagaaattgaaattgccctg
atatatgcataaattagtgtcatctccatcttgtcagttagagtattttttagattctctctatgtat
acatacatatatatatatatatatttatatatatatatatatttgtgtagctgtgcatgtgtgtattt
ggactaatgggtcaaaggacagtactaacccaattcaataattaaagaaaacataattttgagaatta
gctttatggtaattgtttgacttaaatgagtagatcagagaagaataagggctttcccttatttaaac
aagcttcatttttttatccaaacatttacttagctgattaagcttcacttgtttattttcttcaaagc
attcattcaggtgggtactgagtaaactgaaatatcacaccagggaacttcaacaccatccaagtctt
aaaggcttcacttgttcacagttggcatttagtgaatgtctaggctactgataatattgtgagtaagt
tggcagggatcataagaaatgataaaatacagttcttgaaaatgttatggtttgaggaaaagatctat
gtttggaattagactgacttggattcaaactctggctgtacctttgggacaaggtgttcagaaactct
agcctatgttttttttctgcaaaatgatcctcttttccaggattcctgtagagattcaaagatatgtg
aatgtttagaaaaagaatagacttttgatcattgttaattcccttactttccccaattagacttgtaa
gactgggaagaaagctacacaaaagattgaacaaattatagctgacagaccatagcaaaagatacagg
gcaaaacttaaaggggaaaactacacattaaattattttaaaccattaaatagcactaacttttgtca
gatattacaaccaaacaccactcaaattaaagtaaactgaataaaatgcctgtttttttctgtttact
gatgttttcatttgcttcattcatttattggaagatataaaatgtgttagacactgttaggtgctgag
tgtataaaaaaatcttattaatacaatttaaacacgcacacacatatatatggttataacaattgatg
ccatgtatgtactgtttatatgcctatacattattccacagacctggggggagggggatgtagagtct
taccagaaccataggaatcttctcacatcaacatttccttttgaagtttgttcatgaggcaccatcca
gataatactaccatctgcaatgtggcttgagaagatgttagatttttttattacacataataaggctg
taaagtatttctgtatttaggtagaggtatgtaatacaatatgtatataaaattacatatccaataaa
atctggtgttaaataaggactagcttctatgataatatagtctaaaggcttttcatttggtgttatag
aaattatgtgaaatatgtttcctggagtagaattattcgcatttcagctctctgacagtggaagaaaa
gctagagggagaggtgaacaagagagggagcataatggacaaagctttgctggaagccaaaccaccac
ttcatatgtcaaatctgacaggcctcccattttaggtgtgctgtcattgaagctttcagctgcacctt
gcctgtggctaggctattttcaaagattaaaatgcgaaactggaaattaaatgcaacttaattcccaa
tttaaatttccattatttttgaaaagtaaaagattaaaagaaatgtataattgcaattctggtggaag
aggtaattataggaaaggtgggatgtatttcaagtgggggatatagcttactgcagcagagaggaatc
taagctatcattcttttgaaattggtctggaaatatgttttcacatggaaaatatactatatttttag
gaatttccttgtcatattactgtatccttttctgttagaatataaattctgaattccctattccactg
tagatctgcctccgattatattagctcttctgaagttatcaaaaaataatgagatatacaatattcca
tatatgtcaaagcaattatttttaggttaagtaataaaccaatgacctttaacccggtaatattctgg
gttgttcataaaaaaactatattcaggtaataatgtctttccacttaagcaactgaaaaaatacacaa
tacttaacatttggttaattaaatacctactccagacaaaaggattttctgttttcaagttatcttag
caagctgagcaggaagcaatgatatatccaatcagaatatccatggaagctctgctacagtttcaaaa
agttctcatcaggcagcttttaaaatgcctactctgaaaatggtccaggttaaagaacaacagcttcc
tcgtcagatagcagtattgcttggccatgtttcttcctagcacaaaaaagtacctgctcttctctgag
tacctacattctaaggactatggcttacataaaacagcatgggttggggcaatttccagcacactgct
cactctcgaaaacgtatgatgcaggtgagagtaatgtttttgtttgaatctgctttcactcgtggaag
atgaaactacttgcaaagatctgtactttagctattatgagtaacaaaagactcctaaaatattgcac
acattgtggggatggagaaccatcatcctgggatttgatggatcctatggtttggctttgtgtcccca
cccaaatctcattttgaattgtaatccccacaatccccacatgtcaagggagagagaccaggtggagg
taactgaatcatgggagcaatttctcccatgctgttctcctgatagtgagtgagttctcacaagatct
gattgttttataaggggctcttcctgcttcactgggcacttcttcctgccacctgtgaagaaggtggc
ttgctccttctcaccttatgccacgatggtaagtttcctgaggcctccccagccatgctgaactgtgt
gtcaattaaacctctttcttttataaattacccagtctcaggcagttctttatagcagtatgaaaatg
gactaatagagacgtgtctctcagaagtcacagtgatgcttgaacggatccagagctccttcttcagg
aaggtcccaactcattctgaagggtctctccaagcccacctctctctgtaaatgggaaaggttttact
ttgagcactaaaacctgccagaattctcaattttcctaacagtgtgttaataaacacctactcattta
gtatccaaaccaggtctgtatttctcaattagagctcaccaggctttcatcataaagtagagcttcaa
attgtctgcaatcccactcctatcaaaaacctagaaggaggtaatatttcagagtaatactataacca
gatgaccacatctaagaaactgctgaccctacgatgtaaccttctgtccatttttccctttggaaagt
ctaggatcttttcttataccagcaagttacaagcctggactacactaacttgctttccgcagaagaaa
acaccatgagttctgttttcatattaagcacttagtctccatcagacatcaatcgagaaaaaatcatt
aaaaatcacattttatatttgatgtatatttctcaataatcctatgtattagttcattttcctactgc
tatgaagaaatacccaagactgggtaatttataagtaaaaagaggcttaatggactcacagtctcaca
tgactagggaggcctcacaatcatggtggaaggtgaaggggtagcaaaggcatggcttacatggtggc
aggcaagagcgtgtgcaggaaaattgccctttataaaaccatcagatctcctgagacttattcactgc
cataaggacagcacaagtatttagctccctcagcacagaaccatccccgtgattcaattacctcccac
caggtcactcccatgacacatggggattatgggagctacaattcaagatgagatttggatggggacac
agccaaaccatatcatcctatttggatgatcaatattatcaaggtatgctcccctgagggggcgtcct
ttttaccatttaactccaggacaaaagtttatttctttgtaaggacagtgtttatttcttatggtcct
attttctcctaagatccagacaccaaaatggccatctatcattgacttaactcctgaattttgcttag
agtaacagatttagtgaatctaaatattttctggctgtggaatgttaatttatacatgttcaagttac
ctttgattcatgtgacagtttgtgccaaaacacactcattatcagaactcagatcattatgttggctc
ttgttttcgttactaaaggaagaaaaacagtttctcaaaaagaaaattctgatacctaggaagaccat
tatacctcactcttttctttatctcatcaccacatccaatattataaaagaacttacaaagtaaaaag
aaaggtgttctgtagatgtagcgcctggcttgtatggtagcttaaatgaacacagctaaaaatatttt
atggctagtgtccaaaacagtctggcaccagacaaaataagaatatttaaaattatattttagagtta
ctttaagaggaagggagagagagatgtaggcaggaggaggaggagcaggaggagagggagagagagag
agagagagagagagagagagagagagagagaatctggggtttctatggaagggctaagaatatgtaga
aaacagtttacaaagaaatatggtccaagaatcgtgtgtacacacacacacacacacacacacacaca
cacaccccctggaatatttttcagccttaaaaagaagaagatctgtcatttgtcccaacatggatgga
cctggaggaccttatgctaaatgaaataagccagaccaagaaagaaaaatattgtatgatctcactta
tatatggaatctttttttaaaaaaggtcaaatatatacagatagtgaattaaacagtggttaccaggg
tcagggtagttgtgaggaaatggggcaatgtaggtcataggatacaaatgattaaaatatattaatat
attaaaagatataatatacatcatgaggactacagttaataatagtgtgtattcaagatttttgataa
atgaatagattatagctgttcttgccacagagtgaaaaatgggtaactgtgaaatgatagatatgata
atgttctccacaatggtaactattttacactatatatataaatatctatgcatcttacaccattatgt
ggtatcccttaaatatatacaataaaatttattttacaaacacatattaggaatgcatattctgattt
ttaacaatagttaacctcattaatatatttcacactatcatttctagtgtacatgaaaagtagtttat
tgacattagttgtaaaaaaaaaaaaaatggtcttgagacttttgggtcagagaatgttctggccataa
ggtaggtttctgcttgcctactagatatcttaacttcgatttcctgaacatcccatcacttcagaatc
tctcaatcctttctaacatccgcaacattgtttttctttctgcatttcttatattgactgatggattt
ataattcactttctctgaaaaaccctgcagttatcatatatccctatccattctggctctttattgcc
caaatctctaccaaaatcctgtcagcacagcctctgaaatatttctcaaagcatttataatctggctc
tcatcaacattttcaacactctgttttatcattccactattttacatcatttcattttcatttttacc
acaatcactcatccaacaaataagtatttagctccctcagtaattagtattattattattaattataa
ctagatgctgagcatacagaagtgaacatgacagacataatcccagcagggatgtcagactttatgca
agtaatcaaccatgatgaatctcatgagattctgagagagagagagagagattgagagagagagagaa
aggggaaccactggtgtccgagttagaaatttgaattagtatctgggtcaccaaaagcttctgtgaag
aagtgatatagacttggccacacaaaactaccgtgaaggtggtggaaatttttctatgcagagtacca
catttaaagagctaagcctgagagtgtcagagataaaggaacagaaagaatgtgacagcagattatgt
ttggaagaaagatgttcaagagaccaagctaaagaggagatggggctagaacctggagggtccttcgg
gtcctgttgggagttttttctctgcccagaagggctttgtcacgtggttgtcaggaaagagtcatgat
tagagctttgattcagagacttctttcgctgaagtgtggagaatggttcagagagaagcaaatctgaa
tggacaaaagaggttattattgtaatcttggcaagaagcgatggtggtcttgactaaaatagttctag
tgagaatgtgacaacaaacctgagaaaaatacaggagacgtaattgacgggggttagtgttaagttga
acgattgcagagttgaatttgaggaaagtgtcatatatcattcccagtttctgatgtcatacacctct
ggagataacactgccatttcttttgaaatgggaaaataataagtgatcagtaagtacgtattggataa
aataatgaatggttaaatgcataaggggagaggaaaagagttgcagagaaagagagtaaacgtatttt
ggatgtgttaattttgagatacctttgaaaaatccaagtgaggggttgggtagtcagagaaatgaatg
tggatgtcaggacgaaaggtgaccgtgatgaactgtatgtcttcctctaagcacgttatacagcttca
tgtcacaagtgactcacttcatgtcacaagtgactcacaaggtcacttgtgacaagcatttgcctggt
gcttcatccctaacctccctttctatactcagctaaaatgtcacctacaatacttcttccttgactcc
accgtccccactttactgatatgaatacattttaataaaatgatataataatgcttagtttgtaaacc
taatgttcctcaagtggtataattatctgatttgtatgtgatcatcaacccaaccatattaggagcac
cttgaaggtagaagatttaggttcatgcttaacaccacatctggaccactgtggatttaactttctac
aatgattgtattcattaatatattgggtgcccactatattccaagtaatatcctgcacactacgtaca
aggaagcataggtcccgtgtgctcatgaaactgtaattttagtaagcagggataggatacaaactgag
aaaggaaaacaatttagaaagtgggaaatattatgcacagaattaataaaaaagagaaaaatcttgaa
aaagtcttcaatacctcacttggaaggtgattttgaagaagaactgatggacaaactagagtcagcca
tgtaatgatgtaggggcaaagcattccgggcacaagggacagcttatgcaaagaccttaaaaatgaac
tagctttgtatcttggagaaggataaagagaactaaggtatctataaggtaattaggaagaggatgag
ttatttagtcccttagtctttgaagcacattatctcatacttcaattgagtttattcttagtgtcatt
cttctggatgcaatatttgagataaatgtcttaatgaacgttcacctccctccgtagtaatgcctgag
tgtcacaaaaactttttttgtttacatacgtagccatctaatggaaacataaaataggaatcaaaagt
tgagtttcatgtacaaaaggtaaggactgtacatgtggtcataacaacttcaaaagcacctgaaggta
acctttaaggaagatacaaaggctaggaaatatctaggatccatgaagacagacttacttaaggtcat
agtgtgtccagagttggttcccgccggtgggttcgtggtctcgctgacttcaagaacgaagccacgga
cctctgcggtgactgttacagctcttaaaggtggcacgaacccaaacagcgagcagcagcaagattta
ttgtgaagagcaaaagaacaaagcttccacaacgtggaaggggacccaagcaggttgccgctgctggc
ttgggtggccagcttttattcccttactgtcccctcccatgttccatttctgtcctatcagagtgccc
ttttttcaatcctccccacgattggctacttttagaatcctactgattggtgcattttacagagcgct
gattggtgcgttttacaatcctcttgtaagacgggaaggttcctgattggtgcgttttacaatcctct
tgtaagacagaaaagttccccaagtccccactcgacccagaaagtccagctggcctcacctctcaata
gcattaagaatatagtttcacgagcatatatgaatcaaaacttacatttgccaattttatttgcttgt
ttatgtgtttccaacatgtcttgtcttagggccaaatgtttccctagagaataactattccaactatc
ttagttgctgtatttttatgcaaccttcaactctccatactaaaatgtctccagaatagaaaataaat
cttttcaaagtttcaaaagaggctctctatatattccccttaaaagtaccaggcagacatatttctag
gtttctaacattgcgtgttgccaggaagtatatccaaaccatcacaagttattcatgtaaccaagcac
acttattggagtgcttctgcttctgttcttgcttgaaattggaagctccttccaggaaaaaaaaaaaa
tatctatagaaggggaaaaaagtaattttactttgaaaataaaatatacgtgagcaatagttttattc
tgtttttaatttaccatagcttccaaagacaacattgttttatagtaggggttagcaagtgttttctg
taatgtaaacgtaaagggccagagagtaaatattttaggctttgttttctatactctgttgcaactat
tcaactctgctgttagaatgttgaagcagtcatagacaatagagaaatgaagatgtgtcattgtgatc
caataaaactttatttacaaaaatggcaatgggctagttacggcttgagggctgcagtttgcagactc
tcacttcagagctaacagttgttgtcaggagtcacttgtttttggaaacctacaatgaggtactataa
caccaaaaagagttatcccttcctttttctctctcactttttgaattatgagaagaattagaaatgta
gttaatgataatgtccaaccagtgtaattatacttgttagaaacacagctggaagcctgttgtccagt
cttatttctcctctgtgatcctcattttcagaggttgaagtcataagtttgccatgtctactttctga
caggggaattataataatgtggagtcaccttttgtttgtgactttgacaatgcttcattgacttactc
accaattttctaatttttatgaagactttttgccgaaatgtagactcagtcttctctcttgtctactc
tttctataacaattaacaatgaacttatttacctttttaacatctttttaaaaattttctatacacct
tgaaaatgtgaatacaaagtaatgctgcatcatgtatattgccttattcacacatagcctcttatggt
atatcatataaaaatggaacaatacagcaacaggttgaatgaacagtaatcaggtaacaggaaaatga
gatgtctttaatatttcacttaaaaactcaatttcctaaagcatacatataaatatttggaagtatag
ttagaagaaaaatatctttaaaatattttaattgattagtcttatttataagataatttttaggaggc
tggttgcggtggctcacacctgtaatcccagcactttgggaggccgaggtgggcagatcatgaggtca
ggaaatcgagaccatcctggctaacacggtgaaactccgtctctactaaaaatacaaaaattagccgt
gcatggcagcgcatgcctgtaatcccagctactcgggaggctgaggcaggagaatcacttgaacctgg
gaggcggaggttgcagtgagccgagatcgcgccactgcactccagcctggtgacagagctagactccg
tctcaataataatcataatcataataataatttttaggaagcatcagaaatatataagaaaaagatta
ttttcttaattgctttactaaaaacacctctatgatttttcagtaaaacttgattcttatgtcatgtg
tgagtgtgatctgcctctcttgggatactactgtactcatgaggagtgatttttttctccaacgacct
ctttgtcacgtcaacaggtcacaggaatagtgtaccctaaaaagccacctgccacatgctgctgaaaa
tgtaaaagtacacacatacacacacacacacacacacacacacacacacacacacaccaaaatcaggt
atcacaagctgaaaataaaattgagtccaatttttttttaattgagcagttaatgtccttaaaacaaa
atcctatactgcaacaaatacttagccagatcattctgatacctccaaactgtggtgtattccaagat
acctctatgatctttgatttgatccacagcttttcagttatcatgcaaataccttcaagttttatctc
atttctcagtgcaaactcattaaaaattttcagctgaattcaattttataaacatgttgtgaatgtcc
tctttatataagcaaggttgtaaggaactggccacataaacagaaaattgaataacatatggtttctg
gccttagtgatctcatgtgtgagttaggcatatgggcaaaatcagaacactatagagtataagtctaa
aatggtagtattttataatagaggatgaagagggtgctgtgggatcataggtgacagatataactccc
gttgtgggacttgagaaaggcttcacagtctggaaacatttagttgctattgaacacaaaataagact
cactgttgagagaagggagagggagggcatttcaatcaaattaagattctgtggcatattcggaaact
gatgtttttaaaaagagtaatgtttattacattcctctacataaattatatttctatgtaatatgaat
gacaaatatttaacacaaaatgccttataacatttgaatgaaatccatcatatgacctgttatctatt
tccatttcctttttgctcatatcattatgaacaatgacctgataaattttttataagactttgctgaa
ttagtaaaggattattaagtttagaatgaacaaagctgaccaatcattcaggcaaatttgaccgtttt
gttgtcgcttttcttatttctgaaaccatacaattccctgaaatgaataagtacatatttgataactt
cctaaattaaggctcaaaacactggtaatctactgggctttcatttgttccttctatttgtctaatcc
tatctatatttctttatatgagctatgaaaatattagatttattaagttgtcctttatcttaatagag
aagaatgtttttctatgacattaagaggaatttgatttttttctttaatgatctacttttaattttgg
tagagtagcattgataagatcaatattacacattcttaagtatgcattacatgttgataagataaata
ttacacttaaaatatgtttatcaaatgtatgaatgataaaaacgaattctgaaatgtatgggaaagat
cttgaataaaggtctatgtacatttcaaggatgtctacatatgcaaattatcataatataataactat
tgaatatgattatcttcacatactttctttatttttcatctcttagatgaaattgggtattgttttct
tatagctggaacaaagcattacagagaattcttagtgtgatttcattgaaactcactgttatatgagt
tcaacaaagtttaaattagtccatgacttaatcatcctttataaatcctatcactagtattcggtaag
gacaaagtcaattaaaaaattagcaacagaagcattaaaagaaggattaataaatacaaaataaggga
tgtgatatctttacgtattgctgagatgttagtgctaaggaaaaacttccctgttcataatgtgaggt
gggaaaaagaagaactattattgtatatttctcctctctaaaactgcctatctgactgtgtttttctg
tgtcagccgtattaacagatgtttaattttactcactttagtatataaggcatcataatgtatgaact
atttcaaaggccctatgatggctaattaaataaaaatatattaaatattagctggacaaaataaaata
tgtattaattttggaaaaagtagatcaaggttttgcagatcttttcatatcaatatattcatttgctg
aataagcttttattgtttaccaatattactagttttatagagatgtagatatcaccacagtatgacta
attttatagggacacagatagatagatgttattttattccaatcttatttttacatataacaggtata
aatatgcgcttgaaaggagtatatcacttaggagtcagtcagaaaagtaaagatcttctagtctaata
cagtggttctcagccaggggtgattctgctgcacgctgagggataaattggcaatttctggagacatt
tttggttgtgacaattgcaggagtgttactggtattcatttggtagagacagagatattggtagacac
tgtacaggacacaggaaagtctcttacaacaaagaattattctgtccaaaatgtcagttgtggtgagg
ttgggaaacactggtctggaagaaggaatttactatgaggaactagttacgaaagtatagagacattt
aacaagctgaacaaaggatagtgagatggctcagagattagcaactgtggcatgaagccactactacg
tttaggtaaaaataagctaccatttattcttatagtaataataataataattattattattattattt
gagatggagtttcgctctgttgcccgggttggagtacaatggtacaatctcgactcacttcaacctct
gcctcccagattcaagcgattctcctgcctcagcctcctgaatagctgggattacaggtgtgcaccac
ccctcccagttaattttttgtatttttggtagaaacggggtttcaccatgttggtcaggctggtctcg
aactcctgacctcagatgatccatccacctcagcctcccaaagtgctgggattacaggcatgagccac
cacacctggcccactctttcttttttaattattgagaaatataaaaatatgtcaaaagtaacaggtgt
ggtggagttacagcatgcacataatgggatacagcccattatctaatctcagatggaaactagaaaaa
aaagagaagatctttgctaaagcacagattatgtggaaaatcatttagaaaaatagcttatcacaaca
ttaaaattaaatcctttagctgatcatttttccttcctattttttcttttaaaattgagaagacagtg
agttttttttctttattgtcattatcttgatgtcaaaaaataatatgcacattataagtgggaaaaaa
gataagtcgaaatgaaatgaaacaatgcgaggaaaaaaatgtcacaacactcttcaattagaaaaaat
gacccccatctttcctccaaatagaaatgacgtaactgaagtagtggaactttctcttccatggcaac
tctagagaaggggtagatggcatgggattgtggacagatggacacagaaagaggcctcatttattgtt
attgttaaaacttttacttctagtaatagtgacacctccttcagcatttctttatcaattgtcaatat
tttttggatcaccagcatcaccttctatatgtatgtctagaaacctcctgttatgaatttacacttct
cagagtcaagacagaaatgctgtgaattgggcgataaataaaataccccccttttattgccttgcttt
gtctcttaaagaaagatgcctgttgggggactatgagaatgctttgtgcttctggacctcaagggaca
aatctataataaaaattatgcatagtgatgagaaatatatataatgcaagtttgtagagatcagttaa
cttatcttgtctaggcaattatttctaaacaatgatttcaaatcattaactataatatagcccattca
taccctccatttttgtcaaatccctgtcaccttcaaggacttggccatcccataggctgctctgcttt
taatagaggaagatgctgtaactcttggtaccattgccagttatgaatttatccattaatgaacattg
catttaaggcataggtttatctccttctccaggtatgaacctgcaggattcctacctgaagcttaagg
gagaataaatccacctgggacaatcaaggacagatcaaccaatcagctcaaagcaggtgtgaattaca
cagtttatttgagtgacaaggtagctaaagcagggataataaaagaagggagtgggttgatgtggaca
gacgaactatggctttaggaaatttggtagggactgaaacatattttgtgtaatttatgtgggtctaa
tagcttttgaaacttctttacaagacctgtgtaagtggtactggcatattcatgcatgagaaaacatc
aagggaaaacttaatagttcaaggaggtgacaaagaagagaggaaccaattattttcactagccgtca
aaagcaagaaaataatcagcttgagcccttcggggaaaagataggttaaatattaagtaacagtttgt
tattattccaagtgttttcttaaagttgctcccatactttcctgttttctctgagggaatttagtttt
tttgttggtttttttttttttttttttataactgtcattggtcagagcttgatttgatgccagtcaaa
tttttttaaagagattatgaaaactgcttaaactcttccaaagggaagatgggtcattcttaacatgt
gtttcaagaggaagagcataagagcattatatggtaaggctgaaagcagatatcagcgtttaggggcc
atgaagaggtagagctcacattggtaggatcattgactagaattccagagatcaaaattgtatgttag
tctagcattggggaggacttgtagctagtatcttcattctagcttgggagcctaggaatcaggttagg
catcttgcacaggaatgggccgatgggctaaaatctccttgagagagatgattaatccaggacaaacc
aagcagtcatgccaatgaattactttaacagggtacttcatatcctcatcctttgggcagcacggtct
tcagagatggggcaggccccaggctgcagttgagattctataaactaaggtcaaaaagatgcagcagt
gaagaagtcatgcttatcttgtataaatcatgttttcttttctttttaatgaaaatgtacatttaaca
cattttaaaactaaatattgaccctaaaattccaaccaaaaaatgctacataagtggtatttattttt
gaatttccctcatgctcctcccactgtggggacaaggagtggtggtggaagagagatcttttagcaaa
cctgtgagtagagaattagaaggtaatgggaggaaggtaaaaggaaaacatcatagatggataggctc
acaaacattaaaggccttcgtgcctgtccttcatgcctattcatccctctccagtatgtgaatcaatg
tacttgttaaatattcattcacctcacatatttagcattaaccgtgtatcagggacgttgttagaccg
ttggtttacgatgatgtgtaaaatatcatttgtaactcagactaactggaagtgctcaatataataag
atgtaatgttatggaacactaagtctgtgctgaagacttatctcctttaatcctaaaacaatcctggt
gggtagtctcaatgatcatctccaagtcacagttgaggaaattaaggcttcaagaagttaagaaactg
gaccaacatcacaaaggtagcatcagagtgacagtttgatttcaaagtgtacttgacttcaaggccca
catttccttgcacgtttaatattgcctttctcaggtaaatataccattaaatgtgatacaactctaag
catttgaattacttacaacgtgcagagttaaaaccagcattatttacactatacttcagctcgtttat
aagtgaactattattttgtggactaacctatgaaatgtaaccacattgaattcctctgttaggtacag
gtttggtgattccagggaatagagtatgactgaatgcacaggtaggggtgaagtgaacccggtcagaa
aatttagagagcatcgagcagatcattaagcagctgtctttcaaatgtgcagaacacaactcatttgt
aatctagggactatctgtattgattcttcccagggaagttacttatttttatacatatgtggtgtgtt
ctgtccataataccattctacatggtaatgctcaactttattatttaaaaaaactgctaataatgagg
tttttctttgtatcacagaagcagcaggagcaagttttctttttccttcccagtttttttaagtactg
ccaaggaatgtgattttgtcagacttgtatttcctattaagccaatctgcatgactgttccttctact
agctttacctgttcactcatttattaattcatcaaatatttgtagagtgactattgtgtgccacatac
taatataggcacaaggataaccaaaaacagacaaacgctgtcctttcaaggagctcatatagtaatgg
gaagttaggaaaggagaaaataaatatgtggtatttcaaatggaagtattaaagtgttaagaagaaaa
gagaaactaacaagatagggaaaaagtgacaggaacatgatgttttattttttatttatatatatttt
ttgagacagggtctcattctgttgcctaagctggtgtgcagtgacgtgatcatggctcactgcagcct
tgacctccctgggctcagatgatcctcccacatcagcctcccaagtagccaggtctacaggcatgtac
cacgatacccagctaacacgttttcttttcttatagagacagagtctcactgtgttgcccaggctgtt
cttgaactccggggctcaagcagtccacccacatctacctcctaaggtgctggaattacaggcatgaa
ccaccatgcccagccgaaattgatgttttatatatggcagtctgggcagacctctttgatgtgatatt
tgaacagaaatctcaagagagggagtgtattagcccgttttcataccgctagaaagaactgcccgaga
ttgggtaatttataaaggaaagaggtttaattgactcacagttcaatatggctggggaggcctcagga
aacttaaaatcatggcagaaaatgaaggggaagcgaggcaccttcttcacaaggtggcaggaaggaga
agtactgaggaaagggggaagagacccttataaaaccatcagattttgggagaattcactcactatca
tgagaacagcatgggggaagccaaccccatgattcaattacctccacatagcctctcctttgacacct
ggggattatggggattataaggattacaattcaagatgagatttgggtggggacacaaagcccaaaca
tatcattttgctcctggcccctcccaaatctcatgtccctttcacatttcaaaaccaatcatgccttg
acaacagtactccaaagtattaattcatttcagcattaacccaaaagtccaagtccaaagtctcatct
gagacaaggcaagtctgttctgcctgtgagcctgtaaaatcaaaagcaagttagttacttcctagata
aaatggaagcacaggcactgggtaaatatacccattacaaatgggagaaattagccaaaatgaagggg
ctacaggccccaagccagtccaaaatctatcagggcagtcaaatcttacagctctgaagttgtctcct
ttgactccatttctcacatccaggtaacactgatgcaagaggtgggttcccatggtcttggtaagctc
cacccctgtgggtttgcagggtagagcccctctcctggctgcttttacaggctggcattgagtgtctg
cagcttttccaggcacgtggtgcaagctgttgatcgctctaccattgtggggtctggtggacagtggc
cctcttctcatagctccgctaggcagtgccccagtggggactctgtgttggggctccaaccccacatt
tcccttccacactgtcctagccgaggttctccatgaggtcttcattcctgcagcagacttctgcctgg
acatccaggagtttccatacatcctctgaaatctaggcagaggttcccaaacttcaattcttgaattc
tgtgtatccacagactcaacaccacgtggcagttgccaaagcttgggacttgctccctctgaagcaat
ggtccgaactgtaccttggccccttttatccatggctggagtggctgggacacaaggcaccaagtcct
gatgccgcacacagtggtggggttgggggggggacctggtccacgaaaccatttttgcctcctagacc
tctgggtctgtgatgggaggagccgcaatgaaggtctctgacttgccctggagacattttccccattg
tcttgcctattaacattgggctccttgttaaatatgcaaatttctacagccagcctctccagaaaatg
ggtttttcttttctactgcattgtcaggttgcaaatttttcaaacttttatgctctgtgacctcttga
atgctttgctgcttagaaatttcttctgtcagataccttaaatcatctctcaagttcaaagttccaca
gatctctaggtcagggtcaaaatgatgccagtctctttgttagtcatagcaagaatgacctttactcc
agttaccaataagttcttcatctccatctgagaccacctctgcctggacttcagtgttcgtatcacta
tcagcattttggtcaaaaccattcaacaagtctctaggaagttccaaacttttccacattttcctgtc
ttcttctgagcctcctaactgttccaacccctgcctattacccagttctaaagttgcttccacatttt
caagtatctttatagcagtacctcactacctcagtaccactggtcttaactcctgcgctcaagcgatc
tgcttgcctccacccctaaagtgctgaaattacagacatggtccattgtgccgagccaaaattgatat
tttatgtatgacactctgggcagacctctatgaggtgacatttgaacagaaatctcaaggaaggggag
aaattatccatttacatatttggggaaagagcattccaggtagaagaaacagaaaatccgtagtcttg
aggaatgccgtgtatatgcagtatttttcaaacttgttattttgaaatacatatacacttacaggaag
ttgcaaaagtattaagaaagatcatgagtacccttcactcatcttcagctaatggttacatcttacat
aattatatgtaatatcaaagccaggaaaccaggaaattgatgttgatacaatctatgctttattcaga
tctcacatcttacatagctatgcacaatataaaaaccaggaaattgatattaacacaatctatgcctt
attcagatctcaccagcttttacatgcacttatctgtgtctgtcattctatgcaattttataccatgt
ttagagtcatataacaactacccctattttgatacatggtactgaatagttccagcgtcacaaaggaa
ctatctcaagccaccctttaattgtcacacccatccaatctcccattctacttcctgaatcactagca
acccctaatctgttctccatctctatgattttgtcttttcaagggagttttctaagtaaactcatttg
gggaaagaaaggagatgaattgttctagccacggagtggagaacagagagtaagagtacctattgaag
cagagggagtcattgcaataattcaaatgagaaataatggtgattctaaaccaggaagctttcagtga
aaacaatgagaggtacatggattctgggtatttttggaaggtagcactaccaggtttgctgatgaatg
gggtatggggtgggaaagaaagagaagagcccaggatgagtccaaggtggataaggtgaatagaattg
agaaaatggtagaaggatcaagttagatggtagaggggtaaaggtggaagcaataattttgttttgga
attgttaggtttgaaatcttgttagacatcccagtaaagtcacaaagagtgcagttggatgaaagtat
gggattcagggaagaagtatgtgctagagatgcagatttgagagtcatctgtgtggaggtattattca
aattcaagtccccttggaatgaatggctattcaggcagggtcttcataaaaatgcttgttgcatgcct
gtaatcccagcactttgggagtctgaggtgggtcgaacacttgaggtcaggagtttgagaccagcctg
atcaacttggtgaacccccatctctactaaaaatacaaaaaaaaaaaaagttagctgggcgttgtggc
acatgcctgtaatcccaggtacttgggaggctgaggcaggagaattgagccaagattgtgccattgca
ttccagcctgggcaacaagagcaaaactccgcctcaaaaaaaaaaaaaaaaaaaaaaaaaagcttgtt
gcttcaaattcatgtcagtctgtaaaattatctgggaaggcagtacaaaaactgtcactttgactacg
atgtttctggtgacccatcttcattgatcagtatggaaaaggcatgtctctgaaaatctctgagagtc
tttgatacagcaagaacataaggataaatcattcttctatgttcatggttgtagaggatcttgaatgt
ttaatggcagaatagccagatcacactctggcacttctgtatgagaggctgagggatgttactgattc
accccgagaaatatttactactaaggggacagaggcaaaggggatacaagacttcaccctgagctgta
gcgctccctccttccctatcctgctttcattcttcacattgttttccttctttcttttttattattat
actttaagttctgggatacacgtgcagaatgtacaggtttgttacataggtatacatttgccacggtg
gtttgctgcacccatcaacccgtcatctaggttttaagccccacatgcattaggtatttgtcctaatg
ctctccctcaccttttccctgtgtccacattgttttctttctttttgaagcctctcattcactaggtt
tcaatcctgccttgctagtgttctaactctaaggcctaggcaagttatttcaccgaacttagcctcag
tgtcctcatctgcaaaatggatagttttatgatatcttcagcccttaaagtcaatggttctgacagct
agggtgtactatcttcttggatatcagtcatctcaagcaagccctccttttttggaccttcttttcac
acacttcacataccttagagaacataatacacatcctctttactcagggcttattctttataacaggc
ttcctaattcaattaactcaacttttcaaaaatattagtgactactgtgatgtaaataaatttgcatt
ttataggggtcttagtaacccagaagggagtggggaaaattaatatatattgagagtttattaagtgc
taggtactgtaaatattttcttgtatttaatcctccgagtaattctacaacaaagatattatcattgc
tattatgtaaataaaagaacaaagtagaaagaaacccacggtcttgtataagctcccctagttggtgg
gtattgaagggagtatttcaatctttggtagcttctgagtttttgttctctcagggaatctgccagat
gtccagggcacctgccaaaccctatgaggctataagaaaaccattaagggtcttagattacccagctt
tttgggagttagaattctgaatgaaatttagtgttcctgcagctacaaaggaattgagttagggaagt
gatgactttatctttagctacattggttattttccttataataatcctggcttggtagattagaggca
gcccgagtaacccagaatcgctaaaatagaagtgcgagctcattgcccgctgtccttcactatgtttg
catataggaagcaagaataaaacaagcataaaataggctaactagcttgtcagagctcttcacaccaa
gtctttgtgagttccaataagacactgactattattaaaaagacagagactccacataagtaggaatt
tattgttttccttttcagtcaccaaaggacaatcctctgcataggttagcaaaaaatggtactgatcc
tataatctctaatattaaagtttagatttggcaagctgtacatcttatgttgttcattaacaaaaaac
aatattgattggtatcttgtactataacttgtactgtgggtcaaattccaatacagcaaataccattg
caataacaattctacaaaactacatcaaaaaaacctttcatgtttgagccaacagcctgatagtgcta
aggactttgagtacagtatgctagaagattcttaacagttatttgtcctggacaacaaaggttgactc
cattaaaaacatagccatcagtgtgggattatttccaaatcaagcttttggaaaagtcaaatgaaagt
ttgcaagcaggtggggcatggtggttcatgcctgtaatctcagcactttgggatgctgaggcaggcgg
atcacctgaggtcaggagttcgagaccagcctggccaacgtggtaaaacccccatctctactaaaaat
acaaaaattagctggcttttgtggtgcatgcttgtaatcccagctactcaggagcctgaggcacgaga
atcacttgaactcgggaggcagaggttgcagtgagccgggatcatgccactgcactccagcccacatg
acagagtgagaccctgtcttcaaaaaagcaaaaaacaaacacgcaaacaaaaaaaaaaaaaaccaaag
ttggaatgcaataaatgttcattgaatgaatactgaatagggagtttcagctaatccactcaaaatag
tgctgaatttccagctctaaggtcaatgcttggcatatatatcctgaaggaatgaatggacacagagt
aattttttttctaaaatgcaaattcaattatgtcacttcccttcttaaaatccttcagtagcttcccg
tagcctccagcatattattttgaatagtgcttctcaaactttgatgtgcatcagaatcacctggggat
tttcttaattaactgatgctgattcagtaggtctggggtattgtctgagattctgcatttctagcaag
tgctcagggttatagcaatgattttggcctgcagaccatactttgggtagcaaagacataagccactt
aacttgacataaaagactgtttagacccttagtttctctctcgctctttccccattttgagcttttgc
tccggttcatgtttttccctgaaaataccgtgatcttacattgtctgtctggatgctgaattttccct
aattctgggcctccatgtagttttaggtttgacatcacaaccaccaaaagatttccccttctccctta
atcttggttaatgtcactctcatgtattatactgttaatgaagcattgaggacataaaacttatcaaa
tattttatcacaatcaatgatggcaccagtgataacatccaaatgcctgggtgagtaaataagaggag
aataggggacttgttgttaaactaagtttgcagagaaaaaatgtactgattataattaaattggatgt
ttatttgttatgacaaaaaaggagctagagtcttttaatccaccccttggcaccactgcttatctcct
tgtaacatacgtttgattcccatgtctatttcttccatatgggaaatttcagctccctaaacatcacc
aatacaacctgttgataagacaaagttaaatttattgcttactatggtaagaaagaccacagcctgga
caaagctttggtagtatttcataaggagaaaggtgaggttggatttcattgggagtatgaagcttggt
ttaagattggtctttcactgtgggggcacaattaggattgggtaaggatcatggtattacaacttagt
ttggtggaaacagcacagtgaagatttctagccaagaggctcagagactattaaggtgtgaactctat
tgatgttttttgttgaagagttgatgggagtttggggaagttactttagtgaacagtcaaattatttg
cctggccaagagttatctgtaataggaaagttatgctaatgaagacaatggaaaggtaaaccatgtta
atgtcgacagccagctatgtgagcataaggggtaggtagctttggtcctccatgtccaaactgtttgt
agtggtaagtgatcttcattctcacatagattgaaagcttcctgaggacagggcaatgtctttgtaaa
ctttaaaatatctatgtcctgcacatcacctgccgtagacaagcatctagtaattgacggttgggtag
atactgagggaaaacatgcaccaaataaaaatggcaataggacacaaattcactatcatttggaagaa
taacagtgttttccactgatatttgctacacacagtggggtccacagagcagcagtaccacttgggag
cttattggaaatggagactctcaggcaccaccgcaggtccaatgaattaaactctgctttttttaagg
tcatttgtattcaattattatttttttcttttttctttactttcgatgcatttttctttatttgtttt
tgagatggggtcttgctattttgccgagtctggtcacaaactcctgagctcaaatgatcctcccacct
cagcctcctaagtagctgggatcacagatgtgagccaccacacctggcttgtatcacattaaattttg
aggagcagtgctttaatatctattccattctcatcacttgatgaggtattattaattccacttatgga
tgtggaagttgaagccagaaagtttaaatgacttgtacaaggtcaaacagcttacaggtagttgagcc
aagaggctctcaagtcttctgcctccacaaacccctgttcagctgctgccctacaatggaataaaata
tactaatcccagagggacaaatatgctaaaaatctcaatattatacactttggaaggtgcaggtgcat
tatctttcaattctaatttctctttcaagttttetgatgcataaaaatatgaacagcaggtctgagca
atgtttagatgccgtgctttgatccttttgccattcaagatgtttgatttgcattctgccaaggaatg
tctggtaacctccatgatgcagaccacaccattagtcaagagagagctgacgtaccttcatctgagag
ctggctggctgtgagctgctcagagggaaaggatttctatttacaaattgtatcgattatttataaat
aaaagttccccttgctttcttcagttgtaaaatctgcagttagagagtcgggaagaagatcaaaactg
catacatttgcatctgccaagcctgataactagttccagaattacagaaatggtgctgaaatagcacc
tcaagtaccaggctctatcaaatttaatctatccataaggcaactgccaattatattttagagaaaaa
atgtagactgaaaagatagacaatccaagtagcaactcctgtaaaattatatgcccataggagcaatc
ttgaagatataaatattggtatgtttctccttcatttatcatttatctgatcatttgacaagtattta
ttgaatgcctgttaagggtgtagatatatgtggtgaggctgcaggtgtaagtaggtctttctgaggat
atgcatgaagttgatgttcataacttggagatgtgtgtatacagactgaggattccttcagtggatat
taagaagtggagtaataggcagtaaagaatacactagtcagttgtggtacataaacacgtcagcacca
cttaggtattaacttcctgttttgttttgtgtgtgcttaattacgctgtttattaaacaagcacatca
taatctgcagatattgtcataaacagcacaataaagcctgccacatcagaatgtcatctatcaaatta
ggtgtgttcctcagctgtcccgataggcacacacctgtgcctgtaaataggcgcttggcggagattgc
ttccaggtgtggatctgttgggcgaccttgggatgtagggcactttggaaccttttcctctagcttca
ggaattaacctctgggcttggttccatgccagcttgcattttgctttgggacagtaacatgtaaagaa
tatgcctgtgaatttagggttactgagaagtcctcatagaagaagtaaaatttccttgaggaatggga
gtcttttattcaatccaggtttaatgcaaggcttggtgaacagctccagaaggttaataattgcgtgc
gtgtgtgtgtgtgtgtgtgtgtgtatgtgtgtgtgtatccttttgtcattcaaaagtatacgtataca
cacacacctgtacagctgatgataaatatacattgtatcaatgagttcaaatgaagtgtgctattcat
tcactgaggaatgggctattataatgaactattatgatattagaaattgtcagggcaataagcaaata
atacatacggttttcaacaaactttctaagtattgttatcagtgggtttgcttaaatctttttttaca
aatttatttatttttttgagacgaagtctcgctctgtcgccaggctggagtgcagtggtgcaatctcg
gctcactgcaaccactgcctcccgggttcaaaagattctcctacctcagcctcccgagtagctgagat
tacaggtgtgcgtcaccatgcccatctaatttttgtatttttagtagagacgggttttcaccatgttg
gccaggacagtctcgatctcttgaccttgtgatccatctgcctcagcctcccaaagtgctgggtttac
aggcgtgagccaccgtgcccaggcaatagccccattgctcagtgaatgaatagcacactttattttaa
ctcattgatataatgtatatttatcatcagctatacaggtgtgtgtgtgtgtgtgtgtgtgtgtgtgt
gtgtgtgtgtgtgttgaatgacaaaaggatacacacacacactcttattaaccctctggagctgttca
gcaaaccttgcattttttactttcattacagtgtgtaaataatttagcaaattctaatttgaacctga
tatcaattgagcatttaatatttagccaaatatttatcaagtgctgactgtgttctagatgctggggc
tgcaatttcgaaacagaccattgaggccctcatggagctcacaataaatgatcttccttaaagtatca
ggtctctggtttgttaccgtattttttaaattgttaaggaaagaaaaaggccctatctttttgtagac
aaacatgccctaagtgcttccagaaataatctccatcaggtaatgcagactgtgtgtggagtgaaatt
gagtccaatccatgatccagcagagtttcagcccaggatttctttagagcctttgctacacacaaagt
tggctgatgtgccattcagcatcccagcagctctttctcttcacactagcaatggcaaagctttgtgc
ggaggcattgctggctgctctgaactaaaagcatccgtggggaccgaaagaggtttttgcacacctta
ttaaggtaggcaagtgtgtctgagtgtgtgtgtgcctaaaagctggaagacatctgttgagaggaaag
tgctcttctgtgggtctggcagcttttctgtaagtcttctattctgatgcaggagcgtgtgagcagtg
ggtgggaggagatgctttggtacttggaatgctgaggtccggattaagtggtattgtaatagctagtt
agaggcagaataaaaagctgggaatcaaagcatttaaaaatgcatccttccattatttgctctcaagt
taaaccatattcattctaggggaaattaaaaaaaaaaaaaaaacacagcaagggcaagtagcccaaat
ctgtaaggtctttgagcttctctgttcgtccagcttttgaagtcttcctacagccaatttgtttggct
cctctggagggggcaattcatatccacttccctctcctggagcatttctttcttctatactccatcag
ggaacaatagagtttaacagtaacaggcaattttttttttttttcaaagcttgtgccctcttctgcgt
ttaaaggtgttttttaagagactcctgctaggggaatcttggcgcctgtgtgttaagacggcaattaa
cttttagtatcagtgcttacattaaattttctctctttctgctttactaaagcagtcattaaaattca
gtgtgagtaccatgaaactttatcataaaaccctgctttgcttagagaaccttgattgttttctgaaa
gcagccttctcagtttatatatacatagctgccttccttggaatatcaaattgctttgtgtcacatta
agaaacactaggttgaacctctatactgtgttttatctgagaaaaatactactgcaaaaagtttgatt
tgttcaagttttaggatgaaaatttctttgtaacaagttatttgagttgcatactatgtcatcgtata
tctctttagttcaagtaattttgcaattaacatacggttatgtaaagaagataatgatttatttttta
tttatatttttaaaagttattaagtgaggttttcctttcagtaagagtttagaaaaaatagccagaac
aagtaactggacttggaagataaagatacctttgcacttctaaattttacctttgtacacttcggttg
tgatttaatcattgaaatgcctctgctttgaagtaaatgcatcacttatggtgtatgctgtgttttaa
taaagggaaaacagttatgggttctctgttgcacatttgaatgttgttattttttgctgtatttaata
acctcttttttctcttgtgaggtttactttggaaatgaggcatgttcaaaaataggctgacattcagc
ttctatgttttaaatttaaatgctgtctgtgttttatcacatctggaatgtgtggggagaaaagatac
caagttttattatttagatttaattgtagaattgcagattgatatttttcaatgcattttcattatag
tttctgccatggaggcagcgtgagggctttcaggaagatggagtggtgtaattaccaggtgcgcacgt
tcattaatccttcctggctagagaaagcttcaagttcttctccagtggcccattcgtaaagctataaa
tatctaaattgtgtcagccaagaagtcacacagaatggtggctctttttgagttcaatttcatgcact
gttgctttggtcttgtgaggaaagctctgaattccttaggatagtcttggttgtgaagttccaaaaac
aaaatatcaaatcattaaggatttaatttaaaatacatactcttctttcacaaactagatgattgcag
taatgtggattataaatttttttttttgctttatttctttagagctcctctttttattttgtatgatc
aagattatagctgagattttggtgatttttttaaaaagatttatggcttatggtccatcagtctctcc
actacttcaaacctgtgtacccctgtatattatctgcagtactggaatgtttgcattgtatgtggaag
ctatatacgatttggtaaaaaataacacttaaaggtcttcgctaagagtgcttatttaatcattaaat
atcccttaataaaaataattccagagatattgtctgtgtacaaacttaaaaaaagagaaatataaaat
actgtgatgtgaataaaatgtatagcaatacactccaataataccattcttatgttttcccttgttct
caactgaaataactaagctaatagagacgtcagtaaggaatgtgttgtttcttcataatacaactaca
aactcatctgataagaacaacctgagagtgaacgttaactttcctcattagaaagattcaatttaaca
catatatacaaatacatttttaagataatgatatttgcagagtttttgtattctatggagtaaaggag
aattatcacatattcaaagtaaaggtataaaatacatcttaatgttttacttaaattttaaagggtcc
aaaatatactaaaattctttttctaattctttcctatgtttaaacgtgccagagtcattggaaatagg
acattctttttcttaagaagattttgcccaaaatatttaaaactattttcttttcccttgattttaca
atttcaatattcatggatttttctactttaaaaataacagtagtttttatgatcttaaaacaaatgtt
taagggcactttcgctctctggagactataccatccacatatttattatcagcaaaagaaagggcagg
gcatacttttatttgaagttgagtataaaaatgtgtctgtgtgtgagtgttattaaaaagataagtga
agagacaaatatagaatccaggaacattttcagcctggcttttactctctctaaaaatctaatgaaac
ccttgagcatctcttatctcaaggtacattaggaactgtccaacactatgatccgatgggagatcagt
atattcatataaagaagaaaatttgttgttagtgaaagtcaagtcttttaaaaaaataatagttacag
catttgcaatatacaagcataatagatttactcaacgcccaccccccatctttaaaaaatcaatttcc
gacagttgtctactttaaaattgaacatatttgctacctggagggaacattgtaatgtagcccatatg
tggtatgcatcctgaagaaaacctgaaattatagaggaagttatcctgccttctttcttctgttgaat
gagttaaaatatattaacaatttgcctttcactttgtatttatcattttgtatctttgcatatttaca
tatacattcatgtgtacaagggcatatatactcacaggtcagggctatttaaacagctatttatttga
atatgccagggaaaatctccaagatataaagaagcagttattagatactatgtcagtatagaattaac
agccatcttttttaagatggaagagaaaattaattaattacatacaatttctaacctcaagacatttt
ctttctggagacaaggaatactgaggtgctcacgatagtgaagactcaacaagaccctaataaaatag
atgaggataagtaaaactacaatagccaataaaaaacaaaaaacaataaaccatgtttcgctggcatg
ttggtgagtatctctgtaatatctgtcaataagggtctctgtagatttggagtaatgttcaggaacta
cctgtactagagaagacagtggagaggactccagtggctaaattctgctgcctttgcttccagaaatg
taaataataaggaggtattgtggcatttcctggaagcagtagtcttgtttcatggtctgactgtataa
gaatgcctagagaaacataacctcagctgactaaactcccttgatgattgtcactttgtcactgaact
ctgaccataccttttgcctccagaggcaaaagacgggtgaggaagtgatctcctcatctggtttttaa
acaagtatataactagagaactggattatctcctaaacccactcttgtccctggaaaaaggggagtca
tcctatccgtttcttagccaatttatgtatactcttagtttgagagcatgagaaggaaaactattttc
ttttcttaccttggctgggtttttaagaatttatttttagtttaatcaaaataatattttaaaaggta
gtaagcctctcataagcagtttgatctgttctaaaataacttcaatttttctttttttaaactttctt
ttatcttacacacaaagtataatagtaatatgtactcactagaacaaatgaaacaggatggagtcaca
tagagaaatatatcatattctccctatcccctcccttaatattaacatttaggtgtcatgtgcttctc
cattaattttcattgcaaaggcctaaattttcttccaagagtgaggagtagcagcacggtagtttgga
cctgatatagctctctttccctagccttttgcttaagtgctttcctaggggctgactttacttaccta
aagatgtttcaagcaagggctcacatttttggtagcagaagacacttactgattgctctcactaataa
ttttgaaaggaatgtcaaaatctgggaggatcatgaaagaaatatcagaaatttcctttcagctgcca
ttctccttaatactgttatcaataaattcagcatctcatatgtgatagcaaaaaaggtgctgcctttt
gttcttgcatcctgaggttcttacctaataccatggtagcaataaagatggtgagaaaattgcttctt
ctatggtgttcaggtcctgaacgagcaccctcacctccacagacggtggcaggtattcaagcatttta
cagactttggagttaaatatagcagtgttattctaatttaggtatgccaccaccagcggcaccggcaa
ctgcaataggaaaaatgattggcaatgccagctatctgatgttttcatgtgccaggtgctgtcagttc
ttcacagtattacattccatcctcacaacaagagagtgccagtgagtgttgctgtgtgccagtgccca
ggctaagggctttgaacacattaccctgttttatcctcataactttccacgttatttttattcctgaa
tgaagaaacaagttctctgtagagatgctgtcattgatccactcatatcctttcacatccgtttaaca
ttttccctgctgtgcttttactcccaacaactagctccctaatcgctctgttggagggtggccttgag
gctgccagagcctatttggtctgtgtaaagagagagatggatctatcctggaatttatgtccctgtgt
gtgggaagcccttaatcaatgactgctggttgcagacacataaatacgtgagctttcttgttcccaac
tgagaaattcagaagtgtgaatggcactgccaccctgggcttttatgccatatatgtgtttggtctgt
ttcccttcccaatctcacttcattttcccttaccagtgtttcttgaaaacacatcccattagatcatt
tttgcatgaagcttcatctcagaacctccatttagggaacccaaactaagatattctctaaaatagaa
actttattgataaagtttccaaactgtcttagtagatggccaatataagaccaagccaaatctttctg
ggtccaaattccctgtctttaattaatagactccattacaacacattcttcaatctttagtcagcaaa
cacttaccacgtgcctattttatggcatattatatttataccatagttaggatattatggttcatgaa
tattttatatctgtacacctgaaattctattgacctctctgggccacagttttgcatctgtaaaatca
gcacaataatgctacttatctcatagagtagacttaaaaacgaatgaaatgatatatgccaagtgttg
agaatcacaattggcaattactcatgctcattaaatattagctgtttttatgagtattgtttcatttt
cggtgcataatatcctatgcaaagaacaaaaggtattggtataggcattgaaacttgaagcatagaag
aaaaagttaattaaccggtgccccactagatgcctctaactgctggctccgtgtatccctttagcctt
ggctcgtcacgagaaaaccttggagacatttctgctggactcagcagatcaatttaagaaagatgaat
gacatttttcttgaaatgtattcagtcatagctgcctttttctactttcatattttggagttcttaga
aaaaattaaggactcctttttttaaagaaaatggtataaaagaaaatgcatatcactttgtcacttta
ttattgtaacctcatcaaagtattcagtgtaaagacagtagccaagtgaactcttcttgtaatgctcg
gaaaccattttagcaatggtaaaattgctgcaatttatattcgtcaaattgcatgatttgacttattt
tagaaaagttattaacttctgaagagaatgcttcagaagcatttaaatgagtacaagttatcaccagt
gatatacataaatttcatttcaaaatatacttctagaaactgtacttagttagctatagtatttgtac
aaggattaattcctatttcattttgtaggaatttatttatgaatgtctatggcctgccagtgtaaagc
agacttagagcatcatcttttacaataatctttttttttttaatcaaaggggagatattctggtaaaa
caaaacaaaacaaaaacaatagtttattctgcatttttattaagtccctctgtaagtcatccctgaaa
tgggatatgtagagtcttatatttatttatttctcagaagcttattggaggtgatatgaaggatttta
agaccctactaactaacaaaacaacaatttaaaattaattttcaaaataccttaacaaatcttattct
ccttattttcaaattctttaacaatgtttttcttattactaacataatatcttctgatgtagtcataa
taatatctaaaatgacaggtctaagtaacttacatggattaattgagtcttctaaatagtaaggtaga
tggcactattacttctatatgagaaatgaggaagtagaggtataaataagaaattttttggccgggtg
cggtggctcacgcctgtaatcccagcactttgggaggccgaggcgggctgatcacgaggtcaggagat
cgagaccatcctggcgaacacggtgaaaccccgtctctactaaaaatataaaaaattagcctggcgtg
gtagtgggtgcctgtagtcccagctactcgggaggctgaggcaggagaatggcgtgaacccgggaggt
ggaggttgcagtgagccgagatcgcgccactgcactccagcctgggtgacagagcgagactccatctc
aaaaaaaaaaaaaaaaaagaagaaatttttttgagtgtatacagttagaaaatggcaaaatgggaatt
cagacccaaacagtaagactcaaggatacctttcttatcagtatgctaatatgaaaacctaagcatac
tagaaaatctaagtgccagttggaaaccagaattaacattttggtgtgtaactttctggctgcttttt
ctatgctaacaaacatatatgacatacaaaaatacacacatacacaaattcctgttcactacttcttt
tatgttaacatcacaatgtaccgtacacagctgtattattttatatttgatttcatattttttctaaa
gtcagtgtatttgtcaaatatcaacttatctatttaataggaatatgggatgatctttgcttatacat
acatacatatctatataaaaacaaaatcaagtattttaagcgttcaccagaagtcatatgtcaatcag
taaagtatataattttttgctgccaatgacatatatcataaaaacgctacctatcatagaatgaaaat
gaaacacagcaatattgggacacctattctcaagcaacagctttgtgatttattagctatctcacatg
aaataactcattaacttggtattccaagcagcaaaagaaggatcacttaggtcacttgcaaaataata
caaagctaggtttaggggtgggttgcgcttggtgggatgtagatgaaaccatatgggcccttgagttt
ataattgctgggatctgcatggtgggtatatggatgtttattacagtatgctagtgagttaagaaaga
agaggaattattattgacttacatcatagagtttatgcaaaaattaaacgataatttatttttaaact
ctagaggtataggtaccatcatgaagggacccacagaactgatgtagccagtaattattggagctgga
acagatactctgctgtcagttgttctggttttgtggtcattgttcttgcctttgcaagttaccaactc
taagaccttgggcaatactttaagtcttggttgtctcatctgtaaaatggggagagcagtaagtgtct
taaaggtttattctcatgttatatgacttacggtatgtaaaacatctgcgtttagacacatagagggt
gcttaatggatgattgctctcattattaggctacatctaatctatgaatttaaaaactgtatagaaat
atgtgacagattctttaagagccaaataccaactacagtgaaaaatacttaacacttgctgagctctt
agtatgtgtcaggcttaactaccttaatgctcatagcaatcctataagataggtactcttgttatcct
attttatatcttctaaaattgaagcaagggaagttaaataataggacaaagatcatacgctatctatc
catatatacccatctggctgtctacctgtctccttccatccatccatccacttattcatctacccatc
catccactcagttacttctctctctcccaccatccctttccctttccctctccctctccctgtctctg
tcactctcctttacttatctatctatcgatggatcggtttatctatcatctatctatctctatcatct
atgtatagttgttaataacactaacattttataaattacaagactgaaaaatgttttcattaacttat
ggtaacaaaagaccacattgtgaataaaaaaagcagtaaacacaggtctctgcacatatgaaagagat
gtcctaaacaggaagagatgtcctaaacagtagggatacatagtatcatacaatcaaaacatggcagc
cctataaaacttacaaagcaatttcatgtaagttatttcatttgactcttaccacaatctatgaggtt
actatttttatttttctcattttacaggttaaatttaatatggcttccaataaaaaattagtatggtt
aataaa£atcttgacgtcttgctcctataatcctaccgatagtttacagtaattagtaaaataaaata
ataggaaaaatacctttgatactagtattaaattataatcatatcattaggtaatttcaatttgtgat
tttcaagaatctgtaatatggtagcttcttcctactgacatgtttgaattcattttaaggcttataat
tcacaagtaatctatatattatctaaaatgtaaatgcacattcacatggagataataaattagcgtga
aatggctgtattttgctctctataatttttaacatacaggaaatcactgttgtctcaaaaatcaagga
aatatagtatttgaggtgaacttattctttctactattaacacattttaatatagttctctcacagtg
caacagagcaagaagctttcagacacatttgctgctgcaaggagcatgctgtgctgaacttaaaacac
cttccctttcaaactccttgggactgtttttttccaagagacttcaaatgcactaaatttagcatccg
ttggaggcacacccaggcatattatagtgaaagccccaataactgaatgtgttaccactattcacaat
gtttatgtgtgtatatgccttatctatgatgtattgcaaattacaaaaattgtgttattattcacagt
aacaaaaacacttccagcaaatttctaacagtgatctcttttgaaataacttacatacatgtgtcatg
ggtcttaaactttgtcacttttatgtttccatcatgttgttttagccagtgagggttttgtttggttt
tcatttatgattatatactttcaaaaaatagatttcaaagtgtgaatttgattgattgattgactgat
tcattgagacggtgtttcactcttgttgcccaggctggggtgcaatggtgcgatctcggctcaccaca
acctctaccacccaggttcaagcgattctcctgcctcagcctccctagtagctgggattacagatgtg
caccaccacgcctggctaattttttgtatttttagtagagacaggggttcaccatgttggccaggctg
gtctcgaactcctgacctcaggtgatccaccctcctcagcttcccaaagcactggaattccagacgtg
agccaccgcgcccagcccgtgaattttatttttgaaagacaagaatgtccttgcctaattgcataata
gtttaacatcatgaagactaaatatgctttttagccatgacaattttatttattattgttttcatttt
taattttctcaaagatcctcatcagtgtactctttttggtcttccttataagcgtattttaacaggac
ataataataagataaatcccaactttttaaagttgtatccgtatgtattactttaaagtgctattaat
ataaacgaattagaggcaacttttattcaatcagattttaagtaattttaccaaaaatatggccttga
taatgtctctgtaacaggttctctgtaatatacatgctgaggattggtttgtctttgcttttgatact
attttaattagaaaagtaatggggaatccagacccttctcatttaataatccagagaaaaatcagtcc
atgttctaatagtttaaatttttctactaaaacccatgtgagaatccatatgagtggaatggagagga
gttcagcttcaaagttggcagatttgagatgattctatggcaacagaaatgtgcttgagggaaatcag
ttgcggcatcttctataattgtgtcacctagattttgccttaggaatttctagatttccatagaacat
tgtgacctcaaatgctttatcttaataaagaaataaaagcagattagaagaattatttgcctacagtt
tgtgggagatgggcaagtcttaagagtttattaggtacccagaacgaaacatattttcttgggcctca
taatcacattgaaatacaaggatttagttatacacagtgaccagttagtgaatgacagtcttcagtat
ctagtagacagtaaacatataaagatgtatttgtggccgggcacggtggctcacgcctgtaatcccag
cactttgggaggccgaggcgggcggatcacgaggtcaggagaccgagaccatcctggctaacacggtg
aaacctcgtctctactaaaaaatacaaaaaaaaaaaaattagccatgcgtggtggcgggcgcctgtgg
tcccagctactcgggaggttaaggcaggagaatggcgtgaacccgggaggcggagcttgcagtgagca
gaggtcaggccactgcactccagcctgggcgacagagggagactccatctcaaaaagaaaaaaaaaaa
aaatgtatttttacttttaactacagcgagagaccctggcagcctacagcatacaattagtgttcatt
atttagattgcatggatttaatgtgaggggtcaattacttgtctaaccagtgagcctagcctcttgct
caatactgcctgcttcatgagggtgaactgtgctggagaaatatattacaggattatctgcagatttt
ttttaaatgagtggttaagtcaaaagttcttgtgaaaattcagagtaataaattattatgaagttgtg
taactaggtaaaggatagtttcttttacacgggtaaagattaacatgaggaggaaaactttagcaatg
gcatttaattccattcaatatatttatattgagctcctttaaaaatacagggccttgtggtgggtgct
gaggacagaacaaaaaccaagtaatacatgaacataacccttgatttcatgatctagtagacctataa
aagttgtcgatatctgatgaaaagaaaatggtaaagatattccaaacagtgtatgcaaatccagagat
aggatggaggggctctacctgaaggatgatgataagaaaaccgtgttgagtgaagggtgatttgtgga
attcagataaaatatcagtcttgaatgctgagtgaatactcaatgattgactagatcccatggacagt
aatttcttcaattatgacgatgctagtgtttatgactataactatcattctccatgccaggcactttg
ccatttggtaaatgtatagtgtgctattctaacaagcatgcacagagcttttactttaatgtatccat
gagtttattggggttcagaatttaggtaagctttgcaaggtcgtagcatggagtaaaatatctgaaat
tcagacccatatctaactaagttcaaagactgtacagatatttctcctcctttgtgcagagaaggata
ggaatgcttccatattatcatggacttagtcagatgttttaaaattataatgtcctgtgttaatgaag
aagggatgatattcagtgcatattcttaaccgttactttgcttaatgctctcgacttttctgtgagat
ggatagtgtagataaaatccccaaggggactcagcaagtgcaagtaaaacaatgaaactttaaagccc
tttgtcaaaacctctctttttctcagaggatggaagggccgtaaaggttggtgaggaaggatggacca
tttcctatgtagtcttctgacaatattcaaacaaaaggagagtcagcaaatcccccttgatgtgggaa
gttttaatacaatttgcagagtgtctctctggagtagacatcctcctctgcaatcgtgtcttctatat
agcctcagggctttgggtaggtaatcctctccaaggagagtcctggagagggctgtctaccccccttg
caccatcctctaacattattctatagctcagctccttgtttctgtttcctgccttgtttttgtctgag
tctgcaattatgatgtaagcaccatgaaggaaggtatgttgccagtgtttgcatcagcatatcccccg
tgtgtagcagcgcaagggatatagtgagccctcaatgtctatttgtagaaaaaagaatgaacgtatca
acgaaatctgatacatattcattgtgtctgttatctccatctctcttgtcctgccttgttatcttgcc
attttcacaaaaggccccaaggcccatcatttcttgtgtaacttccagagtgttaatttttaaattaa
aattaaggctttctacatgagtgtctattatttgagaaaccatgcaagatcgtgtgtgtgtgtgtgtg
tgtgtgtgtgtgtgtgtgtgtgtgtgtgttgcactctatattatattgaattctggattttttcttat
aaataaaattttaaaaatagttctttaaaaataggaataagatgttttaggaggcacagagagcaaag
gagaataaaaattgcaggtttggggttgtgcatactaattgccattgagtaaagagagcacactgagg
ccatttagaagagaattaacgtgttttgtttttgtttttgtttttgtttttgtttttgtttttgtttt
gagacggagtctcgctctgtcacccaggctgaagtgcagtggtatgatctcggctcactgcaacctcc
acctcccgggttcaagtgattcttgtgcctcggcctcccaagcagctgggattagaggcgcccacaac
cacgccaggctatttgttttttttttttgtatttttagtagagacggggtttcaccatgttggccagg
ctggtctcgaactcctagcctcaagtcatccacccgactcagcctcccaaagtgttgggattataggt
gtgatccactgcacctgaccttatttttattcatttaaaaatattaaatgttactgcatagggagtaa
tgggcttaacaatgaggtgaccaaaactcctatgtaccatgcagagcaatgtatcaaatgtttttaac
tataaacttctcaaaaacataaacctaattgttctgcagctgcaggttatatctgccttgtttgagca
aaatttggtggtgaaaatgccttgcttccatttttccttcaataactgatatggtttggctgtgtccc
cacccaaatctcatcttgaattctactcccataattcctacttgttgtgggagggatccagtgggagg
tcatttgaatcatgggggcggtttcccccatactgttctcatggtactgaataagtctcacgagatct
gttggttttatcaggggtttctgctcttgcgtctccacattttctcttctgcttccatgtaagaagtg
cctttcacctcccaccatgattctgaggcctccccagtcatgtggaactataagttcaattaaactac
tttttcttcccagtctcaagtatgtctttatcacagcatgaaaacggactaatacaataacctatata
attttgaaaagtacttgtctaatagactttcacaatagaaactatatccttatcaactttgaaaagtc
attgcttaatgcctttggataactgaattttctaagattattttaatttcgaaagttaaattttatcc
cagtgttgacgatttttgtatgctacttttaaaatattttgtcagtgatttatatctatggtgcaatc
ttgtaaaaaattaacaatgcaaatgtggctagaccatttaaaaatcaatatgttataattcagcccat
ttaatcactttagttaaacatcttaggaacaactcagttccatttgagagaagacacagttttctaga
tgtgtgttgtggcatcatattgctttacaatatcttacataaggtgaattcaaatcatatcattgaat
ctgttttaaattctgtcatagcttaagattagtgactaaatattggcaggtttatggaagtaggatgt
aaacaagacaaaaacaagggtggaacaagtaattttagtatattattcacttgcacagagaaaagtca
ttcacaccttcttcagctttgtgaagaaaatagactaaaatcctgttgatatagcaactatgttttcc
gtttcttgtataaaaataaagaaaacttcctattaggaattagccagacattttaattttctctcttc
tttctctattttcccttacagtctctttgaaggcaggcaaaatttctataaagttttaagaatgtttt
aagatttttttattgtgaaatattcatagactcacaaggagttgcaaaaacagtacagagatttcctg
tgtatacataacccaactttccccagttacatattaaccaaatacagtatattaccaaacccaataaa
ctgacattggcacagtgcaatcaactagactgtagaccttacttggatttcacctgtttttgcacatg
ctcttttactgtgagtcattatctgttattctatgacattaaccatgtctatagatttatatagttac
taccactatcaagataaagaagtgtttcatcaccacaaagtaacttaaaggattatttttataaagta
atgacaaatgtgtcaaaagccattcctgtgttatatagcaagtatgttttgagttattaaaactcact
gatcatgtctttcagtgtcataactttgggtttccctccctaactataataatcctgatgaattacag
ttgatgaatatgagaatatccaactcttcctgactctataaatatattgactgagattgtaatattta
tggtgtcttaaggggcgcttgttttattatgatgatgtgaacatgttgagaatagtaagaacagccca
gtttagcaaacaggatatgagtcttctatatccagctcaatcgttgccccaacaggggacatctgcct
ttgctacttaattttccattctggaaaatgtgaagtgtatgagaatgaataatcgtctccgattttcc
agcacataataatctgaggagagcaggtacagcaatttaggagctgttttcttttggtttccaaaaaa
agttccgtccagtggtctaagttagtcgtttactaagtgatagagcaattggctatgctttttgaacg
gactgataattatgtggatgcagcaaataggatatagacaatgcatctactccattacagtaaaaaag
actctgatagcagttaatccacataccaggcacttagcttaggcacagttggaggaaatggaatggta
atagactgtagtatggcatgacaggagctgtagcttgagattcagaattccaactctgcctctcaata
tttgagtcctcatggccaagatatgtaaagtgctctgtgcaggtcttggcaaccatccaccacacact
tagtatgcaatatctatctttattagtcaaggatctggaaagctagttgatgagacaaatgatagaaa
caagagttcattagatgaaataaagtaataaatgatgcaagaatttaaaaaagatttagagaaggaaa
gggaacagaactcacatgcaagtagagcaactgtgtatcagataatgtgctagctgagttagaaacca
tgtctcatattaccctgaaaataattctgcaaagctgtaggtgttatttttttcatttgacaggtgaa
ttcatgaaggcttgaatatagggttaagtgagttgtttcaatgtagttattgattcaaatcaagatct
gaatgactctaaatatggtgctatagagatttgaagtaggataaataggatttgaaaaaaagaaaaaa
tatatagggaaaggaattggtacactgtagcagtgtcataaatgaagcttcagttgtgtgattccaga
tgatgtatgtgaggcctaatcaaacagctttgtggaatcaaaatttctgctcttgtctccaactgggg
acgagttggctcgggattaaggtgggcgaccttgggaagactagagtctaagcaggactttagtccct
cataagaattatatgaggatgtatatttgcatacaaattcctgggcccaccgagatctgccaaattgg
aatgtgtggtgatatcacccagggaaacatagagagctgttataattagtcatgaaatatttagtact
gaaattatagattatgttaaataatcacttataggggacatagcagggttggcaggttaaccatacag
caaacagggttgtaagtcagggcctagagaattttcaagaggcaggaattctgcagaatgaaggcctg
gtctcatgcagcaccatggacagctccgaggcactcttgtttctccaaaaacctgaaatcaaaaactt
tgcttttcatcatgcaacatacccatgtaacaatcctgcataggtactccctagtccaaaattaaagt
tgaaaaaaaaaactatactttcatttgaatacagttctcttcggctttaccagctctactctggaagg
aatatcttttactcaatgaaaggccatcccctgttaatgcctggccaggttctccttatcagtcattc
actatctttgtgtgtgagtgactaaacatataatgctatgtttagtggatggagtaagattacctttg
cagaggttgtactggcttacccctttggttcttgtagttttcttctattagagttttttccatcccta
ggtttctatactgttcaaatgggtttaagattcttgaaggtattcctctgaccttgtaatttatgctt
gtctcctagcacaacttttttttgtaaaggaggcaccaactatgtggtttgctggcgatggcatacac
aaatcaggtgggaggaattaatgagagcagcaattccaatatctggttcttcaagattaacttgtata
gtttaattcagcattctaaataagcctcatagatttaaaaatctagaataaacccacatttttaaaaa
aagttttatgttatctgtgctgataatgcacgctgtacataataaaatattattttcttttttttaaa
tttattattatactttaagttttagggcacatgtgcacaatgtgcaggttagttacatatgtatacat
gtgccatgctggtgcgctgcacccactaactcgtcatctagcttaaggtaaatctcccaatgctatcc
ctcccccttccccccaccccacagcagtccccagagtgtgatattccccttcctgtgtccatgtgttc
tcattgttcaattcccaatattattttctaagtggcagtggaagaaacatggaaagttctacttcatc
catcggtggattagaatttgtataccatgagatgattaattttcaaaaccagtttgaatctcacaaaa
taatgaccctgttttttgaaggacaaggcagaacaaggaactaggctgtgccacgttcaagtcacaat
ctctaacatttgttttgttttgttttgttttgttttgttttgttttgttttgtttaatctctgttgct
tgttcactttctcttgtaatctgcattgatttgctacctggctatttgtagattgacttcggctgcca
ggaatggaatgtttttcataaaggaacatatgccttaatgaaagtaccataagaagggagtagagtgt
gaccaattgcctaggtaataagtagtgacaacaatgatattattctagtataaatggaatcagttttt
ctttgcccagggggcatgataaagaaggcctggctggtatatactaggtgggacacaccaacagtgcc
tagaatgtcaatggatcaaacctgagggaaccagaagttgaaaagacatatcccaaaagaaagcattt
gatgtttaagggttggcttacttagaacacaatgaaaaatattactaaaattaaaactatgattttag
ctatttttaaatatgacaaattaaatagcagaacattttaataaaacattacttaaggtccacaattt
tctgtaagtctaatacatgggtcattaaaataaaaaattccccatgatttatggaatcagattttttt
aatacaacgaattctaaatggttttataatgccaattccaattaatatcctaattataacatgtcatc
cagaagggttaatgactaaattttattaatatttgttttctatttattttgatttgtgcagtttatgt
gtatagtaacgatagctgcaaattagataccattagcattaaataaggtatatattttaatagaaaat
taaagttaagtatttgagctagcctaaaatattcaacaacttaaatttgttttttgtggatcacattt
ttttgagacaaagtcttgctctgctgcccaggcttgagtgcagtggtgcattcatggctcactgcctc
aaccatccaggctcaggtgatcctcccacctcagcctcccgggtagctgaggctactggcgcacgcca
ccatgcccagctaattttttgtatttttttttagagatggtgtttcaccatctggtctcaaactcctg
agctcaagcaatctgcccaccttggcctcccaaaatgccgagattacaggcgtgatccagtgcactca
cccctgtgaaccaccattaaatagctaataaaagatgcatgtcaataaaaataaacaacttactagaa
tgattatgtgaaaatcatttattcttccaaagcatgaattttcaaacacaccttttgttactgtttta
agaagggaatcatttccatatatttgcatgtaaatcacttttagtctcagagaactttccataaaagt
ttttttattactgctgtaaccgatagagctagtggactattaatttaaaaagctgtacataaaaacac
atctatagctcaaataatctaggataccttttagtttggggaaatgtagatgaaaatgaagtaattac
agaatccttgttaattttcagatttagacagtctaggcaatatctttcaggaatgaagagatatgtgt
tttttggcatcttggtagagtatattcccattgtaattcttttgtgaagtctagaccagatgtggcca
taaaaatagacccctactacaataatatatttcatagataatccaataaagtcaaatcttattgcagt
aggcttagaactctgtttgcacccatggaatttatatcagtttttggcaaatcctttcatctctgagg
atactttttcatctcacatataccctattttctgaacattttgccttcaaagtatacctcatttatca
agaatttctctttattcatctgacttatacaagtggcaataacaacgtctggttcccatgaagtaacc
agtgaccctttgaaataatatagcgctggaagaaagaaaaggaaagggagactgatcattcagcaact
ctttaaaaccatgtcaccgttaaacacatagtttattttatcttttttttagaattgtgaaaacctat
attagcatcttcacggatgtctcctttgtttacatccccgcttctgtgccttgcctgcagtagaaaaa
aaaaggacatgtgtatccctattccccattgtcttctcattctacatgagaatgagaattcttttaat
ttcttctctatctacatgaacccacttccattatctgtttgttcagttctttaaatgccctgaagcta
gctctgtgactgggcagttgaaagttctggacttagcatcaggttaatttgaaaaatacttattgagc
caccaccatatgtcagccactactgtagatgttttgaatgtgtcagtgaacaaagcagaaaagatgta
tgccctctggattcttgggggtctcaaatagtgaaagacagatacgataagtatattgtatagtatgt
tcaaaagtgataagtgctgtgaaaaaaaagaagaagggtaaaataagagatggctcatgctggagtac
attccaattttaaatagggtatcatggtattcttcattgagaaggtgacatttgagcaaagatctcaa
agaatgaggcatggggttgaatcatgtagatatcagcagtaaactcattttgggttcagtaaacagtc
aatgcagaattcctaagccatcggtttatctgctgtttggggctggttatctgcagtgtggctagagt
gaagtaagtgagagaggtttaggagagaatgttagtgaggtgagggtggacctttgaagccattgtaa
ggacgtttttctctttctaagtgtgagaagatgatgctgactgagaccagggtgataagaaatagtca
tattctgaacgtgtttggaagtggggccaacaaggatttctggatgaattggataaggggcatgggag
aaaaatggagtcatgaatggctccaacgtttttgctctgattaactggaagggataaagttgccctaa
actgaaataataaagactatagatagaatggggcgattagggaggcattaaatttggatatctgttag
acatatcaccagatatattgaataggcaattgaataaatacctttagagttcagcaaaaaaggtccag
gttggacgtttaaatttcggaggtgtttgtataagataacatttaaagctgtgatatcagattgtatc
actaaggaagaatatagatagaaatgacaacgtgactaaggactctaacattaagaggtggattgaca
aaggagaaaacagcacaggataatgaaaaggaatgatcagccaggcatggtggctcacacctgtaatc
ctagtactttgggaggccgaggcgggcagatcacgaggtcaggagttggagaccagcctggccaacat
ggtgaaaccccatctctactaaaaatacaaaattagctgggtgtggtggcacacacctgtagtcccag
ctgctctggaggctgaggcagaagaattgcttgtacctaggagacagaggttgcagtgagccaaaaga
ttgtgccactgcgctccaacctgggcgatggagcgagacttcatctaaaaaaaaaaaaaaaaaaaaaa
aaaaaaaaaagaaaagaaaaggagtgatcaatgagatgggaagaaaaacaaaagtgtgtggtgtcctc
aaaaactgacgttctattttcaaaacctacattttgggtctccttttactatatcctgactttctagc
tatataaccaaaaggagaaagcagtaatttttttagatataacatgttaataactctaagggtattca
atgaatctgaataattcagtggtataatgtgaaaaaatatagtattcataggaaaaggaacagaagtt
agctcaggaaatgacttgaatgaacaccgaagccaaatctccagcgcaggtccacgtattatttgtct
cagtggttgaattagcagcaagattccttagtaggatgaaaaaagatgttgtgagcatctgtatctac
atgactgaattaaattcctccaacaatgaaatgtagttaacgtagtatctcgaaaagaaccctaagtg
gaattcagggaacctaaattccaaccatggttttgctgctgactgattgcattcacttcaaatctatc
attaacctccttgtgcctcattatcctcatttcaccaaataagaaaaatgaaatattcctccttccct
acctcactaggatgttgtggatttaaatgtgtgagaagtgcttgagatgcataaaatttgatggagtg
ttttattcatgaattcaaggcatctgaagtaatttgaccatgatggacagttgcttccttgcacattt
tttagagtgacatttccgttactgacccacccatttatgcaacatgttgcctaatctaaatttaggtc
aaaacaaattgaccttataggtaagcattatatctattaatattgtatttttgtattattttataata
ttcatcattcacctattttctcatgcaatatatgttactgaacacatatagattaaaaagccttcatc
cctaaataacaatgatgggaccttccatttttatatccctctggcatttaaaatgtgcttttatagcc
atcatctccattgatctctcagtcccttgaggttgatatgacagatatgctttttccattttaaaatt
acggaactgacagtctcagatgactttaccctccaactactgtgtgaagaagcagggtctggcactga
ggtcttctgacatccagtgtagagcactatacttcacaatatggccattggcttactttattacaagc
actaaatattttccactgaatacgtaatacctagaggagaatgtcgtgtaaaacagcagcagtagaac
agaggattaaatgacccattttcttgaagttatcttagttttaaagggttttttcttcatcactaatg
accatccctgactaagaaattattctcataatacatgataatatctgcgttttccaatgcgacaagaa
tgttaggatgtctatacatgatcttgacaatccctagctccatcacaatgtgtccaaattcattttat
ttggctagacaggcatgtagtcttactttcaatggttggctctgctggatgctatgtgatctagaacc
tgtcacttaccccttctaaacttcaggaattttttatccttaagataacaagaaaactcgtacctgtt
tcaaagagctgtttgttcaatcacctatccattgattatcttctatatgccaaatgtttttctaggtg
ctgaattacaggaatgaatcagaagcaaaaagttcttactctcaaggatcttatatgctaatgaaata
gatgttaaaaaataacaatttttgtttcattttattttattttattttgttgagacagagtctcactc
tgtcacccaggctggagtgcagtggcacagtctctgctcactgcaacctctgcctcccgggttcaaat
gattctcctgcctcagcctaccgagtagctgggattacaggcatgcgccaccatgcctggctaatttt
tgtatttttagtagagatggggtttcaccatattggccaggctgttctcgagctcctgacctcaggtg
atctgccctccacggcctcccaaagtgctgggattacgggcatgagccagcgcacctggcattaaaaa
gtaataacaatttttaaatatcaatatgtcttatacagaaaagtgagcagtgtggtagagtgtaactg
gaatgtgagttgagacataacaccagacagagaagccagagaaggacttttgtttgaggaaatgacat
ttgaaaaggaacctgaatagtgacagaggcagatacctaaagaatatgttccagacaaaggaaacaaa
aagcgtgcaattgcatagtcaacttagcctacttgaggaaaagtgtgagtggattttggtgatggaga
ggtaagtgccaggagatgaagggagagatctggcatgcatcagatgatgtgcagtcttccgggacgtt
gtaaagagttgggctttttttgtttataaattaaatgttaagccattggggtttttaaccagaggagt
tatgtgatatgatctatagttaaattatgtttgttcttggatggagtgtgcattatgggaatttatac
agaaacaagatttcacatatatatatatataaaactcagtgtcaatagaaaataataaaaacaaattt
tatccattgataattctggcattgatagtagtgggtatggtggtaataattgtgtgtaacactcaaac
tttctgaaaacctacacttgatctgtaaatccaaaagtatatgtagcaaaagccataatctgctctta
tttctgcaccacttgcaccagtgtggagtgataaggcaaattattcaggcacctgtgtaagccttcag
tgtcctcacccccttgttataactctccactaatattacattggtaaagacgtccctgacctatatgt
cactgagacctcaaagaaaagagcaaagctaaagcgtaagggggaaaaaagccagcttaaaaagactt
aaaggtttctgggaccaaaaaaaaaaaaaaaaagtctttgaaaaatgagaaaggaaggatagaagaaa
agattctcctttggtcaatctggccaacctttggaaataaaaagtattgtgttgcagctaataactat
ttgtcactgcaggcacttgctgatgtctgccctttaaaatgacccaaactcgttggcctcgaaatcag
aagccaaggaaaaaatcttggacataatgttttctgtagaattaccaattttctctctctctctctct
ctctctctctccctccctctctctctctctctccatatctatatatatatagatgtatatatattttt
tctgtaggaactaccaattcctatctatagggactgattgagaagtcccttatagcagtttttctttg
gcttttaggatgcaatgattattggtgagaataactctttcatttcacatttgtcattggcttatttg
aatgtaatcctgattcaatcgttatgatctcctttaagtaggaagagaagctggtattacattgtagg
attttaattttgtactcatgaaacttttgaaaaacattactcatactcttctgactgtcaaattggcc
tctaagaggtccacatctcaagaggtatcaagcattggtaactattttttggtgttgttttctcatca
taaaatgtacttttattaggtgactttggaaattttattgaatcaatgcatgacactgcctcattcta
gtaatctgatgaagcaaagctgaaaaacaaaatttgaggattgtcagtatatatacttttatttgcag
tcaagagttatgctgcaaaaatggtttattgaagtaacaaaattttagctgatatattaatctgaaag
atacagtatacatttttagtatggaaaagatgaggaaaaggaggttctctttcctctaggtatctaga
gcaaactgtaactgtccttggtatttaatttttggctaaggtactgagattagaggtggggccttaga
tatgattaattgtcagactgataagctagatatttcattgagtttctgttgtgctctttctttcagat
cctctgttcgatgctttgttataaagatttgggcatttcaaaatcttctccatatctggtgtctttcc
aaacagcaggtcatagactttacacaaagaggaacgacacaggttataagtagaagtgttttaaaccc
tgagttcctatttcagttttgctttcttaaacatattttccttatgtgataaatgcgagtgttgaatg
gtgataaataccacccataggctttaaagcctaaatgttgaatttgacactgagagtttaaaggcatc
atgaaaatttctccagaactaatgttcaagcaatttaggttttacaggcaactcaatagttttgaatg
atgtagttattttgaaaaagtcaccataaaacgctatgtttagggaattggtactttgcatttatcag
aagattgtaaatgtcaatcgattggcttgctatttggaatataattttttaaattatagttcaaatca
ttaggatttaattcatgattttgtactacaaactaaatctatgaaaaatatcagatatttattttaaa
ttagaggcatgtaaaggaaaatataaattttgaaatgccattttactggatttttctcttcagcccac
cctaggcatttgttacataaaatatttctgaggaagtcttccactgattttgtaaacaaacatgtttt
attgaacagttctttgttgactagattaacattgaccattgtatgcaatgcattctcaaaatcttaga
agctggttttctttttaatcatataattttacttgttttacagtgaaattaatgcatgtaaaaagtat
acctatatagaaagttaaaagaatattgctaactagttactatacttccaaattgcctattttctgtg
tcttgcattggacagtagtgattacctctaaaagaaaatggatggtctttgtttcattgaagggatgg
ataatggacataactggcattcttgagcaatgcaattgcaaatacatgtctttgcatttatggtccaa
tcatcttcttactatgatagcatataattgaaggttcaaataaatgcctcgtcccttcctgtggcata
ttaaagagaaagaaaaattagaaatactttcaaagctacctcacatactaatggtagagttgtttgag
tatttaggtgatttaacaaagctgatgtattttattatgcttgatcattgaggaaaatttatttatcg
gaatgcttttgagagcatatatattgtcagagataaacacagctggatattaaagaggtaaaaacaga
ttttattcaatacctcgtgaaattaggggagagctgagatccattctaatttgtgcagaggcgacttg
gttgttttaaggcaagaaggagggagaaggagtgggggttcattcgagttagagaagtaaaaaagtac
aaagggctggacagtgtaaatgtgattaggccagctgtgttagctggaagttattgaagttaggattc
tatcttcccacagagaacaggagacagaggacttatccttcttgatgatgtcatttgaaaagaatggc
tttcaggtccttgagtgagagacacttctgatttccaagagctacatgttcacaattgtaagcccttt
tgagtaaatgttctaagaaacggaggtaagagtcctatcaacagatgtgtgttggctagaacaaacat
taaattttcctggcagcactgagctttctcaagcaggcacttaagggaaggctagggtcatcctaggg
acatggccttctggggctagaaaccatactagagtttagtcaagtcttagtgcaagggtttggacaga
gttgttaagtgctgagagttctgtatttctcactgtcacaaaggaagatcagaagctcctgatacttt
tttcatcagtacaattgaatatataaatcctatacacaaaaataaactaagcttatacaagcatattg
gtcaaggaatgttgctggccttattaattagatagcccagttaaaagaagaattttttaatataatta
atgttaaagtaggatgatagtatataaaacgtgtctactgtcctgaatacaaactaaactgtttggtt
tagcatttacctcaagatctcttaatatcccccaaagggtccctaaaaccacaacttatctttgtgct
catgaagtagagaagagacagttaatagacatttctagctgatagactgttgtagagcagagaacgct
ctgtgtttttgaaaattaaacatatgaatttgcccctcttcccctattaaggaagaagagtttcttaa
ttgtgcgaacacatcaagtgaactattcaattagatttttgtgacccagggtataaacatctggttaa
ggttacatatttcaaaggaacaaaacactagaaactcttggttttaaatctcatggctggaggataat
ttgcagcagagatttatctggcaagcatacagaattgctgagactgttctaaagatgtaagtgtgggt
gtttgtgtcgtgaaaatagctgtttacatctattaagtggataccgatggttgaaagtgccgtctatg
tcaagtttttaccaaatcaacttttgcctcactgtgtcagaccattttacctaatcaacttggactgc
taatgtcctttcccctggcaccactatctgtctcttttgcaaagcacagaaacggcatgcatgattgt
agtttataaaacacatgtaccaatgtggtctacagcttctgttgagttcgagagggtcagtttctgta
atctcttctggcacagagtcaagaacagcttcactttcctcctgctacctctctacccgtaagtgtga
acccatcactttgctaacactcaggaaggggattacacaaaatagagcaggagccctctgacctgaat
atgcatctgagccctagccatagagcttctgattcagtagatctgggatggggcctaaatatttgcat
ttttaagtgtataagtgatgctgatgctgctggttccaggaccacattttaagaaatatcgataaagg
tggagaattaaactgcagctcagaagacctgagttcttgccccagcttgacttttacaatctagcaaa
tggataaaactcgcaggacttcagttctcttcatctacacagtgagtggttagattggctttgtaatt
taaaattaaacagggtttgattctgattcactacacaaggttccaaagaaggaatgatatctcctttc
atttcttcactttgtcttctgtccctaggtaatcttatctatgttcctgatttaacctaactaatgtt
tctgcaaagcttctaatatttacatctccagccctgaaactctcatttgaatgctagtcttatataca
tacccccctgcctaattgacatctccacttaaatgtatcagaggcaactcagactcaacaaggaccaa
actgaatgttcgaccttgtccttcaaacccgatacacatccaggttcctccatcccagtgaatgacac
tatccagttaagcaagccaaaagtctggattttttttcctcactcttcctcactgtccgtcaactacc
attattaaatctgtcacctggtcctactgatttaaccttctcaatatctctacagtttttctttatgc
ccattagtatcctagtgcaagctaccatcgtctctcattggaattaacacagtaacccccctacccac
cagactgttctgcctacagatagtgtgatatttaataaatataaatctagccttggctagatttctcc
ttcaaaaggttcacattaattttagccttaaaatggtgtgcaaagctttgcatagtctgtcctttgct
atgttggcagtattttttactatccctctcatctgctcattctctgtactccaactacactaactttg
tttttttttttttttagatttctctaactacagtgctgtaatctctttttcctttgcacgtactattc
cgtttgtcagggaatctgctcactgtctccacccactccacacactcacgttttcctgcccgtcttac
cggtcttgatcggttgtcacttgctcaggaaggtttccctggtcaccccctccacaaattgaattaag
tcctcttgctgcatgctgtcctagtgctctttattttcctctcctcatccttaattcagtttgtaatt
acatgttatttgtgtgaggatttgattattatctgtgtcacccactagatattgggcattctttactt
actcaccactgaattcatagaaccacagtaattgtacacaacaaatattcaagagaaatttattgaat
tgatgaatgaaaagttgtaccttaacatgttcctgacatgtatccaaaaaagagctcccctttggggt
ctattaggactttggacctaggtaaacgtaaccctagtttcgctcaggtttaaacagtagaaagtaat
tgggtctcttttgcatgtggctttcctaagggctaaccctgtcttcggaatgagtcaatacagcagag
ctgttgaaagcagactctagcttcggacaacgttggtccgaatcatggttccgtcatttcttagctgt
gtgatttagaataaattaatgttttaaagctttgatttcctcttccttaatctggagatgctaataaa
gccaacttcgtagaggtattgcgatgagtaaataagcataatttgctgtaaacaccttgcagattgcc
tgttgtatgctaactaatcaataaattgaagctcttaacatcattatattagatatttccagcattga
gtatactatcaggcatgtggtagaagctcaatataaagttttgttaaattgaatagattccatatatg
gtatttctacagcattatgctccttatttaagtgtctctaagtattttttaagtatcacctcacaaaa
gacagatgtttaattcattacacatgtgaattgttttagatagaaaataaaataaaaaattcaaacat
tgaaatcaatagtgtaccttaccttaggattacaccataaaatttctaccaatcgagaataaagtgta
cagtctatttcctttctaatacttttaacgcaacaaatgtttattgaacacttactacttctaatcta
tgacagacataaagatgaataaagcatgccacaatgtttaaaggagctcactatatcataagaaagcg
gattcacacagacaactctataagataaagtggtaaatttaggctggcctgtgaaacaaaggattata
ggtatagttaagaggtggaatttattttacttcgaggatttcagttacctttatattctttgtctaac
ctttcatgtttctctttcttcagaaacagagcacctttttcctgacacattcatttccccctatggag
tagagcagttgttttcaaagtgtgggtcccagatcagcatcacggggatggttagaaatgcccattct
tgagcctcacaacagacctactgaaacagaaattcttggagagtggagcccgcagatctgtgatcaag
ccctgtaggcaattctaacgcacactcaagttaaagaaccacgggaagaaaggtccatcctgtaacaa
gacagatttttttcattagcatcaattttgatcatttatatatatatatatatatatatatatatata
tatatatatgcatgctcacaaaaccattcaccttactaggttttagtattccccttcctgtattcatg
tggtatgtatgtatacaagatgaacacacatttacctgagacaaggtaagactacacatgtctcattt
ggggaccagaggctgtaatcttactcaaggtcaaagcgtcttcactgctttctttcactgcttttcaa
aagtaaaatttccatgtaggtgtcatttgttttctttttgtgttttagaaaaccgattaaggggtgaa
gtctggctaaacttagtgtcaggacatttacttagataaaattattttaatttatcttgtaatgttca
atgtgagaagaaaagtccttatgagtagtgtattccttaaataacaacaatttaaaaactaccactga
agtctgtcagagtagttttgcctcatttgtctagataagagaaaaaaggttcacattagggattgcaa
tttgtctgccaaagtgcagtttatttattcagaaacatttagagaggaatgtgtcagttctgttgcag
gcactgtgctgtgacggggagctcaagatgatctcaaaaaatttcacagatggggtgggcagggggca
cagagagatgtatttagtggttcagatactatttagactgtggccagcatttctctaaatgcaatcca
gataacaccttacagaatcatctgggcagcttgataaaagctgtagactcctacccttcatcccaaac
ctattgaatcagtgtctgtgtgtgaagacctagattgtgactggtaattataccaaagtcttagaagc
aactctaggccagtaatactcacatcagaatcagctggagggtttgctataccacagattgctaggtt
agccttcagagttgctggtccagtaactttggtgcaggtccagattttgcatttccagcaagttacca
ggtgatactgatgctgctggccttgatcgtgctttgaaaaccactgctttagctacgctataggaaaa
accatataaggcttttatactggccaatgacttcacaggcctgaattttagaaagcccccttctgcag
cttggcctatagattcgaaggaaacagaactaacacaagaaagctagttaggagctagttaaaaatca
tcctgacttgccaaggaaaggtgctgaagacctgggtcacagagcaaatgcaaaacactaggactttg
tccctagttcaccattaaatcaacttattttctcttaccccctcatattcacgtttactccttacttt
gtagtggttggacaaaaatcaaataaatctgagaattctaaaatgcacacccttgtttattttctaac
tcaaatatgccactgttgtctgtgctctgtcaagatttcaacacatctttttctcctgtttgcttttc
cttttggcatatagtgagtgtgtgtatacacacacacacacacacattttttttgactccttccaatg
cccttctgctctccgcagatacacttctgcattctgaataaaaccgaatacatatatatatatatata
tatatatatatatatatatatatatatgcacacatattttgaaaaccttatttgaaaagaaagctttc
ggaggaaacgttatttagccacttaatcgagtcttttactgagggactttttgtcgtcccctaacttc
ctgtcagcagtccacaggcagcaggaataatgtgggagaagatcaacaggcttatttcaggaggtcag
gggccagtgccaccacctgcaggtggagacatcagaagcaggaagcagcccaccagctgcagggagaa
ctccccacagagcctaaccaagatgaagggacttgtaaatttcaaccctcccttttggcttttgtgct
aaaaatgtgaatattgaggtctgccctgattaagaactagatacattcctctttgtgactgccacact
tccttagcgtattcattttttgtctttcgatctcaagttattattttcaaatgcattgcacgtatcta
ccatggataccattgcaattggaaggagcaaacgttttgtatgtttacttgacaaagagaagtgactg
cccaagccacacagagttctgcacaaatcagtaacttctaacgaacgtttgcacttccgggcttgttc
tctacctatttcagtcgatgcatttgtattatttacttcaaactccaatactaataatgcctcaatca
ggttgcaattgggatttgagcagccagaatttcagaaatttggtttggtccatatctgtgacaggtca
gtaaatcagagaagcaagggtttggttgctattataatacattgcttacctatcaatttagttatcag
ccaaggtggttgttatcatccaaagtggctcattaaccaccttggagactcagtatacaattgcaagt
aaccctggaagttgtaaataatcccaactgaatttgtatgagtttggtaaggttaagtggaaaccagc
tgcttagggccttgattataaatgaagttaggagtggaagaagtaacaaaaccccaggcaaattcatt
aaacattttttcccttcaactttatgctcacgaatgtgttgagactcttctgaatccataaaacacct
ttcagcatcatctgggcagcttgataaaggctgtagactgcctgcccttcatcccaaacctactgaat
cagtgtctgtgtgtgaagacctagattctgactggtagttataccaaagtcttagaagcaactctagg
ccagtagtactcacgtcagaatcagctggagggtttgctataccacagattgctaggctagccttcaa
agttgctggtccagtaactttggtgcaggtccagaatttgcatttctagcaagttaccaggtgatgct
gatgctgctggccttgatcatgctgtgaaaaccactgctttagctaggctataagaaaccatataaca
tggacaaggcaaatgaaaaggttggaattcttctgaatcccaacacatttgtgagcataaagtcgaag
ggaaaatgattcttctgaatccagacacatttgtttaaggataaactgttttttccttctgaaaattt
aatgtctgattctcgttcattcattcatcaaaagttatcaactatcaactataggtaggaactgtgca
atatgctggtgataaagagatgaaagacacagcccctcccttcaaccagctcctagttgaggtggcaa
gtcagctgtataatcaagtaattgcaagactgtgcactgaaaagggtgaccacagggtgtgatggcca
cccagggctgtggaatcagtcccaaaatgaagaatgaaagcagggaagggtaattcagaaagaagaaa
cagttcgcataaagacccatagataaacatcaatcagatgtggttaagacaaaagtaagtttctggag
gctgaggaccttctcagctatatgtttgcagtgcttggtatagggctttatgcatctacatggaagac
agaaagggccacatcacagtggacaaggcaaatgagaaggaggcagtatcagaagatgagggtacacc
ggagatcctagttatatatgggcattgtgttcatctcaggagttactgagtaatgggaccttgactca
aatgaatctcaagtctgtttttgcctaatcttggttttaggactaggattagcatacaaccgcactag
gagcctagttatacgaaaggctgcattgcggacctgatacagttcaatatacatactgtcaccttgca
aatagggttacgttagttctcaagactgccaatcctctgtgctctaatccttttggcttttttttttt
tttttttaactgtctcactctgtcatccaggtgaagtgccctgggatgatctaagctcactgaaacct
ccgcgtcccaggttcaggtgattctcatgccacagcctcccaagtagctgggattacaggtgctctgg
cgccaccaggccctgctaagttttgcatttttagtagagacagggtttcaccatgttgcccagactga
tctcaaacgcctgacctcaagtgatctgcccgctttcctttggcttttaacactatagagcaagggtc
cccagccctggggccacagaccagtacaggtcagtgacctgttaggaaccggggccccacatcaggag
gtgagctgcagggccgccagcattaccacttgagctccaccttctatcagctcagcagcggtattaga
ttctcataggatcacgaaccctattgtgaactgtccacacgagggatctaggttgtgtgctccttatg
agaatctaatgcctgaagatctgaggtgcaacagttttatccccaaaccatcgcctcccacgcacctc
tccccacaaccccacccgcccctgatccatggaaaaattgtcttcctctaaaccagtctctggtgccg
aaaaggttggggattgctgctatagggcgatggttttcacatttgatcctgcatcaaaatttccaggt
gactcattaaaatactgattgctgtgccccactcgtaggagttctgataaggtagctgtggggtgaga
cctgagaatttacttttctaataagttcccaggtcatgctgatattgctttgataaccaaagcaatat
cagctttggttatcaatatataaccaaagccacatagagggggagaagttccttgggtttagcccagt
gtttactgcgaccaccaaaattgctggagcttaaccatggctcagagagttatgttctgttcactctg
taggctgctattccctgtcaccttttgaactatgatggaggggaagagctgccagctcaggagatttc
acttttttctctgcataattgaaaatccagaaacacagggttttgggaaagctatagaacagatcatc
agtgatcagtgtttaataaagtaaagcaataaactttactgtgtaaaataggatactttattatataa
attttgtccccttcccccacctcacaggccaataaaataatatacttcttgtccctgggtgtaatgtt
attggaaacctttgaatgtaggagaggcatgggcttgtaagttgcagaaaactgctagcctaggattg
agaatttcatggataatccaaaaatagatgattttacagttataagccttacgtgaacttgaggtaag
aaaacacaatgcctttatagtcttctcagttgctccacatgccctctgagattctgttctgcccagcc
tctctggttgtcacatctctgggcattaacagaaagttcacatactctttgtctctgatgataatcct
tctaggtccatatagaagatccctatccaaaccatcccccaaacaaacctattggttaaatattttct
ccaccgaaggcactttcttagattctaagtgccctgtaggcaggcttcctctctgatttgggagagta
caaattgcgacaaggttaaatcatagcctgggaatttgacctaaaattcactcttctcccatatgcat
tcatgaaccttctgctggtttttaaaagaagctacttaatgtcagctcgaagaggttggaaggggtta
aaaacatgagcatggcagtaagaagatttatgaaggatctgagaagattatgacttgatcagatggta
ttttgtcagctagccacatttgtgaagacttgaaaactagggaggcttgtccttctaagagggggcac
tgctgggacctggattctgtggaaccgtattagtagaataaacaataacctttgcttgtatcaaatga
acttctattctcatgtgtcttttgacatatttttattaatcatatcactgggacctccttgctgaaag
atatctccgttccccattctgatgactcccaactaggagtgagatcaaatgaagatggcatggaccat
ttctccatgtgacagctctctgtggttgccttttaacacttctaatgccctttctcttaagaattccc
atttgtcgtctggcactggtgctgtgatcaataaaaatgtaatggagtgaggcttagaaacatgagga
aatttactcaagctatccatttattgatgtgtccatttgtgttgtcagggaagaaaaactttttcact
cccctcttaggttcattacttggggggctgcaaattaaactgacgacagatagattggcaatagaaaa
gacaaagtttattcagagaagtatgtgggagctcacagaaaacatagctcaatgaagttagaatttgg
ggcttatgtactattttaacaagggttttgaaaagaagagtgttagaatttcaagccacaaagttggt
gggaaatatgaaagaaactaatgaaaggtaatgtttgttttagtaaagtctgtttatgtaattttctt
ttcccagcgacaacttctcatctctggtgacaggagtcactctttacccctggtgcaagaaactttcc
ttaaaggaggatttaaaacagttgaattatttcagaaatctttgcttttaggcagatagggggagtac
agaaaaagccccttcccgtatctgttgatcctcaaatggctttagctcaaaacaatttttacatcacg
atggcataatgtagatctcttcaatgtgttcatttattccacagatatttgtgaagtacatgatatat
gccaggtacttgggatacaagaatacataagtatgtccctagtctcgtagaacttacactctagtagt
gagctagagaataaatgatattatttattatatgcatacacatatgatttcagatagtgatccatatt
ggaaataaagctggttaagggaatagaaaatgatattgaaggtggacttgtttagattgggtggattg
gcatggcttctctagggggcagtatttgagcagatatgagagcagatattctccaatttgggcaaaaa
cattccaggcagaggaaacaagggcaagggcactgagttcaaaagagacttgacctagccaacaaata
gcaaggattccagtgtaagagaaggtggggaaggaaggaggtgcaagtataggcaagggcaagatcac
acgggatcttgcaggccgtgataaaagaatttaactctttcataattttgacaggacatcattgaaga
atttagaaaaatagagtggagatacctgatctgctttcttcaaagagttcattcatcattgctgagta
gaggttagactgaaatggaagcaatagtgaatacagggagatagcacaggaagccacgttactagtcc
acatcagaggtggttcagactagggtggagtggtggggtcagttagagagctggtatttaggatacat
tttaaagacaaagctgacaggatttgctgtgatgaattagatgtaaagtatgagaataattgagaatt
atttctaagttctttgctggggaaaagtggaggaggaaaaagttagggtacaaggtgtgatgaaatca
agagtctctcttattatcagagtctcattagatatccaagtggaaatgctggaaagaaagttgggtag
atcagtctgaagctgaagacagatactgtgactggaataataacgtaagagttggccggacacagtgg
ctcactcctataatcccagcactttgggaggccaggataggagaattacttgagcccaggagtccaag
accagcctgggtaacacagcgagacctcgcctctacacacacacacgcgcgcaaaaattaatcgggtg
tggtggcacatgcctgtagtcccagatactcaggaggccgaggctgaaggatcacttgagcctgggaa
gtcaaggctgcagtgagccgtgatcacaccgctgcactccagcctgggcaacagagtgagaccctgtc
tcaaaataaataaataaataatgtggcagtcataggcccttagatggtttttaaagacatgggactgg
atgaagtcttctaggaggagagtttgggaaaagagcccgagaattgactgcacctttcaaaacaggag
gaagaaaaaaaatactcaaaggagacaaaagcaacttctgtgatttatagagaaaaccaggcaagtgg
gatgaagaaagtccttcatgatagaatcaaaaacagtgtcaaatgttgaaaatacaattagacaaaca
caaaagaatagaccattgggttttgcaatatggagctcatacttgaccttgataaaagacattttcac
tggaagcatgcatcaaaaaactatttgtggtaggttaaaatgtagtaggaggtgaggatatacagaca
gtggctttcactgtgcagatactgctgctcatgcactaattaaaagacatttgttgagtatctactat
gttgtatccattgctaaatagtaacagctgggtttagtcaggtagaacagcatcaaaatcattatagt
atcccaagataggtacagtaaaatctgtgaaggaatcagagtagtctcttctccaacagagcgtaaga
cccagcttcacggagaaggtggtagattagctcatctgggaggctgagtagaagcttgtcattataga
gggagaacatcagaagtgtggacaacagcttgaataaccttgaaaggacaaaagaggacggtctgccc
tggaaatattaagaagtctcacatgattagacacaagatattaggggaaaggcataaggtgaattgag
tcaatgaggtcaaagagaagctagctggaggaacaggcgatcataaaatgagtaaaagtatatattca
aagattctttttagaagggctacacaggatggataaggggagagagagagttgaggcacagagacaaa
ttggaaaggtgcaatcataaccagagacatgaaaaacccatagaaatctgatgtagattatgtggtcc
ccaaggttgaacaattaagtacgctttcagttgttatgcccatgatattaacatattttataactgca
ataagtgctgaagctaaagataaatacaaacaatgtaattcttattctgtgagaaaatgttgtagctg
gaagttaaacatgtttcttagctaaagaaaaatattgtgtgatctggattacttaatgttataattta
gcaacaaaatgttgacattgagccttgcataatcaaaaaagtagtctattcaataaccacattctcag
aaaaaaaacaagaaaatattagaaacaatgataaattatcgtagtaatttaattcagtattctattgt
tttatttggatttaggaaaggcagaaatgttgaaatattaatatatatccctgtaataatataatttg
tgtctgagaggtaggaatgagggcatgaggtcaaagtttgataatgaacttcaaagctataactatga
tcaggaaattaaaattggacaataaattcctagaatcgtcaggagttgcttgtgaaatcgagaaagga
aaggatatacacaaaaataaagaacagccaatgctctcaaaggagtctaacttttataatagtcttct
gtgttagagctgaactcttctggtttagaaggacactctgttgcctggaaatagggcatggaaaaagt
catcagagtcatgtcatctttcattcttcccatgaacgaaatcgaggccctgaaaagtcacctgtgtt
tgctgtattttattgcaactaagatgtgcatttttaaattgatacataataattgtacatatttgtgg
gatacatgtgatattttgatgcatgcataccatgtgtaattatcaaataaggatatttctgtatccgt
cacctcaaacatttaccattgctttgtgttgggaacatttcacgtattttattatagctattttgaaa
tacaaaatagattgtcattaactatagtcaccctactggatgcaccttgtttttaatatttctgaaaa
cagatacgtctcataggtgatggtgtcacagctgtgcattagttattattgcctgtgcaggtgcaaac
gtaactattcatattgttgtcaattaattaaatagttacatttatttatatgcgtttattatactaat
aaacacaatattgagatagttgagctctagttttgactctgctgttaactagctgcgttactttaatt
tacttaactaatttggctttcaaattcctgataagtaaaattacaacatgagtttctcctgctataat
agcctgagaaatcggtgaaacacatgaattcagatgttgatgctatttaatagcgggattccagatat
ctacttgccattatgggagggagagaggaggtggactggaggctgtgatttccctaggaggttgttaa
aattggccaggtgaggaaagctgagacagaccataaatatgaagcatgatacctagccctcagtgttg
aaagaaaatcaaatctcatctttgtggtctaaatatcagtatgatacaatcctctgtgtagacatatc
ctctgccctattgttttctttctaaaagctaaagcccaggtgtgatcacatccctccgttatttacaa
atttctgatgatgatgattcttctaatatctacattccttaccattaccatgatgtccaaaacctatt
ataatctattcgtctccaagtgccatgttgtggtcaccctatgcaccctctaaacccaccatatgacc
ttcccgctgctacttgaatacagttggccctctacctcgttgtgtctttgcattgcctatttaattgc
ctttccattctctaaatcactctttcgctggaccagcaacatcagcaccatctgggaattcattagaa
atatagatcctcaggcctcatctcagacctgcttgatcagaaacattggagagtggagatgagcagcc
tgtatttttatcagccctctaggtaatttgatgcacactaaagtttgagaaccactggtctagagcat
tcttctttaactctcttctaaaaattattagaatgaattcgagggacgggatctccttgaaagccaag
aacatttctttgtcatctttctgacttcagggcgtagtacactttttggcccataattaaagctcgat
aaatgcattctatgccaataaatcagctaatcaaatatattattcatgcccttgaggtatctgaaatt
tctttgcagaatgtaatatataactatagagtaacaagagaataatttattgccatagataataaaac
aatatcctctgtataataaatcctagcctctgctcaatgggcaaaaacgggactggggtttcagattt
taaaaagattattggtaattaaatcacctggagaagcacttgctgcagagatgggacttgaagcatca
taataaactgttgtttattatgattcggtcagagctgatggaatcacagggattgtgtgaggtatgga
aagtggttgacattgaattccaggctgcacagttgggacttgatatgataaccaaaaagaaagaatgt
ctggggtggtagcaagctctaaatttagacaatctaggcttatcctaaggagaatatagatacagata
actgaagtttgattaaagggaacctggtgtatcacaaatagtaaaaagctgtagttagtctatgcagc
tatcagctagccacataatacttttgggcaaatacattataaaccaaaagaatgacatggcttatctc
tgtaacaaagtggctcattgttctttattctactgttatccttaagaaaaaaattttagtaaatttgt
tatgctatactcaacttcaagaagggatagcgcttataaaaaaattgtttaaagaaacaggcctattt
ctctttgggagaagccacggagaaacgaaaagaatggaacgtgtgtttctgcccagatggcaataaaa
tgtagggtaaatttctgtcttttaaaactgtattttttccatccctctgtatatacacatatcctagg
actgttataaaatgctgcatgcgtatgtgaaaatggaaccttattgggctgtttgatggacctttaaa
atatatttgttggtttggggtacatactagctatgcaatataatccgcattatttcttatgtaaacaa
tggataaactgtttcacagtccagacatttatttggtcactgtttgtagaatgtctattttatttact
tctgaatttgtattccagagatctgccttcaatgttggatacttccactgtaatattctaggagatgc
tcactttctttttcagcatctgacacagtaccatctgcctcctcttttcttgccacaagtaataacaa
ttttataaaggaggatcacattacagaattataggtggtaaactttctaccaccagatttacccaaga
acctgaaacacattttttcaaaaggaaatagaatgtccttcttgtgactacatcggaattttgcttgc
agcattatgctttttttttccccctagtgtagctagccatgtggaactgaagccattagccagctcct
catcctataaatgctattacctgggaaaagaggcagaaaatatactctcttctccagttagagtctaa
aggaagagaacaatatgggtagttgtgtttaccacaaattgatagaactcctttattttaaatgctaa
aaccaaataacttgtttatatgacttcaacattgactatcacacactgttgcatgataacagagtgaa
aactacctctattggatttaagtggggaatctatgtctcattctcattctttttttactgtggaaact
agttgattccaggatcagccttagctccaacttgccacactttgagttttggtttttcacttgcattg
tcacaggaaacttctataggataaatcgaggaagattttactctgcaacgtgttgcagaattaaacat
ttaaagtggcaaaaccttcgtgtgtaggttgtctccccagagaatgtaaaaatgaattgaaggcagca
cctaataggtaaacgacagccaatcaaacaagaacaaatgaaatttgactggcaaaatcaaattgaaa
atgtataacgctgaatctcagaatataggaggatgcatagaaactaagctgtactattataaaagtca
tagccattgaaaaataatgactggttaatttggttttctttacctcatggatgtgaatggttagattt
tgatgttggtgttatttgacgtgtgtttgtcaagaagttgccttagtcggctcgcatttaggataaaa
aaaatattttaagaaatgtttaagagattatgttggagacattagaaacaaaataattatgcagaggg
caggactatcaaaatataatagaaaaattacaccgctcttttatgatttcctcctttttggcatttaa
cacaaaactttatgattacacacaccacgcactccagaaatgcttaaaggaagatgagaggaaaattc
aatagaagtagcaggcatttctgtgaggacagcagaatgatcacttcatctctgtatttttttttttt
caaatttctgtatctgtacaatgtcttttccagctctaatattctgtgatttggtaatttccgcactc
agattttctttaatgaattttgtatgatattacctatttttataccagatattacctggctctaattt
ctttttcaccctaggaaataaaagtatcgggtgaatttcccattttcttatgttattgatacaggtct
ctgttggatatccccacgattaactttcctgcagcatgttcgatggtggcttaaagaagaaaccatgt
atcagagccccttgtctatatagacttttagataaagagaaatacatatcacagaattattctgggcg
catagagtctctaaatgcaaaaaaaaaattgtattgtagctgttgattcttctcagatagattgagtg
tagagagagagcattccaaaaactgagcagaagaaacacagtctgaatcaaataacatgaaattttag
ctaacaagtaaataacacttttttcagaatatgcaaataatattggtttattatgaaaaatgtatagg
ctgatagatgagcatagagaaaaaattataaatatcttctttaatatcactttccccagcaaaccact
tttaacattttgatacattttcatgttcaaacatttcctaatagtcttttttcctgttatataaatat
gaattttaaacattcgtatgtttatgaaaaggcaataagatactgctcttttataacaggctttctga
acttcacaacatgcagtgtattctaacatgctccttgtgttcttaactaataaaaaacctcacgttat
ttaaaaaaccatcttaaacataattatccattaagagaagaggttggggtagagagtttcagactatc
aatatcaaagttatattttctgtaagtattttaatttttaagtgtagctataggtatatgattataaa
accaatagcagagaaaagataccacctttgaatatagttttccttggttccatgaaaatggcctcctt
tctttttgccagtccctcagtatcattaactcatttttctgtaaatgccatcattgtatcacatgtcc
tcaggaaaaggcacttttctcttttaagctagtgtttgttcttgttctaattttatggcaatttaacg
agtaacaatcctgtttctataaatactgtttcctaattaatctattgcattctatccatgagaattta
gatgactttctttgtaagagaaatctctgtagcatgagattcttctttgctcttaaatttcattcttt
cacatttttaaatgacctgatagtattttgttgtatttgtgctgattttttttaaccaatcttacctt
gttgaacatgtaagttgtttctaatatttgcaatgatcaaaatgtggatccaacttcactaaagcgtt
aagaatctaaaacaaaacaaagaacaaaaagttggctgtcatcttgcttggaccaccccgtgagttac
tattttcttgtttccggtcacagttcatcctaaatcatttcagtacacaaaatgttttttaaagtttg
ggacagggggtagagaatgtcaattattcctccaaggcagtcatatgagcattgagtatcatgtggaa
tagttgttacttgtaaagttatggggcatcaaacccagtcaatatgtttctggaattgaaaaagtccc
tggacattctaatgatactgttgttcactttgcacctactgttaccactactttgatctgtcaacact
gcccgtaatggttaattttgtgcatcaacttgactgggctacaaggtgcccagatatttggtcaaaca
ttattctgggtgattctgtgcaagtgttatcagatgagattaacatttaaattggtagactgagtaaa
gtagattgcccttcctaatgtgagcagacttcatgtaattaattaaaggcctgaatagaagaaaaaca
ctgaccctcccctgagcaaaagggaatcgttctgcccgactgccttcaaactgggacatgggcttttt
cctgccttcagactttaaccacaatattagctgttcttgtatctcaagtctgctctacttcgattgga
actacactatcagctctctcgggtctccagcttgcttgttcaccctgtataccttgggagttgtcagt
ctccatagttgcctccataattgcatgagccaatttcttaccacatacaaacacacacagagacacac
acacacacacacacacacacacacacacatataattatatatgtgtgtgtatacatattctcttattc
cttttgtttctctaaggaaccctaatatactccttattactctttctactgccttagagatcttcaag
gccaagagcgtaatcctccatcctggctctttttcctaatcattaatgatcaactcatagccatttag
ctcaactaaaaataatttgttcatgaagctttacactcccacatactgaggaacgtggtacctaagat
caaacagtcactgcctcatcaaatgcattcctcttcaaccccatacaaatgtccccagatggaactca
caccataaaaatattagatcccattgacttttctgctttctcaaggatcattgcagagcttgaaaaag
atggctcctccctttgcctaagcaggttaacttggtgtaaaagtacatgtaagatttggcacaaagga
aaataaatcagttttgcctgggtcctaagaaacatttccctctgcctcatggtaattgtacctgccag
ttgattgcattactcaagtggagaccatgaagtgaagtggtagaacaagaagaaatccctataatttt
attaagtatggtgaaaaatacagatatgtagagaaatgactgggattagatggagcaaaacataattc
gagatcctgatacaaattgtacttcctggctcaagggagggagcagaacattccctgctacatgggaa
taataataaatgcctgataaaaatgcagatatatcatagactacagaagctgaagtggattcttatgg
tcccctactcagacagcctctccttcagatgaagaaactgaagcacagaaagctcatcctagtgtttc
atattgaaaaacccattcaagtctattttaataacctgttaccaaaaatgagggaaataatttaactt
taatgtttcactttgcattacccttttcctgactagacttctatccttttcttgagttgagctcatta
actactatgaaattatggttatgggtagaggttaattttatacctgtccatcttctggcatcttattt
acactaaaaatcatttttaaatggcttcattttaaaaaatattatttcagttgacattttaaaagaca
catcatttatgtactacagaatatgcattttatactctcctttattaattttattattttccaggtag
accaatcaaatgaatcagaaattcttggttagatctattagacagcataagtatgtttttcatcatta
aattaagatgaaaacacaattttactttaaagtgtttgacgtttccagcctttataaagtcaacactt
aatcacatctgaaatttgcaggaaaaaattttgaaagccttcaattattaacattatttcgggagaaa
aagccactttgccgcagaactttcacttttctctcgtgaattaagtctgatacaaattattcattatg
gtgaagtttaaacataatagagtctagctacttccacaaaaatactattcaatgagtttctacattga
catctaactgaccttgtaattaatgttgtacacgatccttttattatatgctggattatcaaatatga
cttattagcagtataaagacacaaagttctgaaatgtaatttatagccatgaaaaggaactgagcttt
gtgtgacagttaaatttgaagagatcaggtgattattatgaagcatgaataataatgcatattaaact
cacgtttttgtttaaatcattaatatgattgttttagaagaaagtctacctctatcatatgggcaata
aaatgtgtataagagcaaacatttgtgtatgtgaaataactcaaattaaaaccagttttccacattaa
ttcttacagtttttaaaatttaaatcatttaatgtatcacacatagctttattcattttaagctataa
atgttacaatttctgtttaagctgttaatataagctttgtaagagcaattctgtataaatatagaatt
gtcattattcactaatagctaccatttatttagtgcttgttgagtgcaaaagtactgcactgagatct
ttgcatatgttctcttaatgttacaattcttacctgaggcatttctgtttctgctggaatatggtctc
tctgaattgaacaagggaggcatttttggttgttatgatgaaaggtggacactgctggcactaacgtg
tgttggtaagcgactagactcttcatgatgcgtaaacagtgtttcctcatacccctgcacattcaaat
agaggaaaaccttgtttatagttaatttcccctagaatgtaaatccatttaacatataaacacaaagc
gtgttttgtgtggatgttttttactggagcagggagacaggagaggaaatgcagttttgatagttgct
gaatttttcaagaatgcagcaattatagaacaatttctagaagtttcctaggagctcttttccatagc
agaaaactaggacttaatagccttgcgactcatggtacttgagtgttccatacaactcacctatattc
aggggacatttgaaaaattctacattaaaggggattcttaacataggcgcaagtgtctggcatcttca
ataggtcttctggtgtggccatgaaaacattcacacgtttcaaagtattttaaaataaaataaaacat
atattgttgtgttatgaattattttctttcttttttatatgatggttagatcactgtgcagacaagtt
tatgagatctattcatttcatttcagggtggtaaatgagggtgttactaaatgttggttctaaaaagg
gagacattgggtattacagaattcagaacagctctaagccctgtgcacatttagcattagaggacaca
ggcaaatctggcctccagtcctggcagcttcttcactatgtatatgatgttgggtgggttgctttacc
tctctagtttttacttttatttctaagctagggctattcatagttctttatcatgtggttactgtgaa
gtagcaaagcacctgacataattagagcagataaaatgctcaacaaatattgcttatcagaaggatta
tgtattacctcccgaaatacatcaaaaatatattttccaattcaaagaatatgtagtacaaaaatcat
gcctaaattaacagagttgcagtagcccaaggagagaagataatcattattgatttcttcttcctttt
tgctaagcagttctctgtctctgcctcctcagttgttgtccatcccactcccccactcccaagccctg
aactctgaggggtttgctgccgtggccggttctgtagtcattgctgtccaatgatgaaaacacaaaat
actgcaacagaacactatgcctgtcagcttagctcccttctttctgctaaatgacactcaatcctatt
cttttgttctaaaggatatcctaaatgaatagccactggggggaaaaaaggttatataagattgtgca
ctgtgtgaaactgatgcaaccagatcaatgatgtgaatttctcttaactatttactgggatctagaaa
caggtctctcaacttagcagtgtttacgaatataataggccttccttatacatacatctgaagccaat
ctgagtcaggaagagtcgtggtctgataaatattttgaaaacttgcatttgttctattaaagcaaact
gtttattaatagtgtgccttattttttaaagcaaaacatttataaacagtagtcattacaggcacttc
agtgtacqgagtqatcaattgttagacctttaggaatcgattgtttcgtggagcttcggcttataatt
gaaatgtcatcagaaggagtgtaagacatagcttcaggagaggccatttatgcgcttttgttttcagc
taagttatagagtcatcatgtgaagaaagattcttctcttagtaaaaatcctttaatggttggaataa
cacttgatatttaatatttctttctactttatatccacatttattcaagtgctaacgcgtgtggggca
gcaatgaagcactttattccaacattatagttctcatatctgcgtatgattatttttcatttatcgtt
agcatatatataatgatgacttttaaagtacactgtattatattcactggaataatgattagctatta
ataatttgaacactatccaggaaattactgaacatgtcctacaagataaacctcgtatgatattgtct
ccaaataacagtgctaaccaagaagagtgctaccaagttcaaaagtaatcacagggagtaacctaaat
gcagctccgttgggttaaaaatagtttctctaaattatatgttccctaagtttgagatcgatttctac
aaggggataaaatgtttttataaattctcagtgataagtcatgtgattaagaacccccaacttttttt
ccaaagacatttgcatctctgatcaaaataacaagatccagtcttagttataaattggggaattttca
tcaaaataaggagctactcgttgcataagaagactagtacaacttaaagccaatttaatttcaatgaa
tgcatgatcagctccattgccaattgagtgtttttcttattcatcagaagatgggttcatcatcgtgt
ttcatatcaactgttctcaaaccatattgcccatttaaataaatatagatttgtctcgaaattctaaa
ttcatgtcatatttcataaatagcctatggtcctatttattactttaaaatattatagatataatatt
tttattctaaagtaactgtgttatacaaccaaattattcatttaaatatgtgactttttaaataagta
aatgacttatttaagtaaagtcattaaaattttccagtctgtccttcatccacctgatctttgaatga
gttaggaacaatacaggaaactaatacaaacttaattttgattacaaaagatgaaatcattctgttat
ttattcaacacactatgtgtcaataaaatcttatactgtgaaagaattcgtctaagtccatttgctgt
tgcttgtaacagaatacctgaaaatgggtaatttacaaagaaaaggagtttacttcttacagttacgg
aggctgagaagtccaaggttgaggggccacatctggtcagagccttctcccatccaagtactaaccag
gtcgaacctcacttagcttccaagatcagataagagtgggcgcgtttaggctggtgtggctgtagact
tgttagagcctttttgctcatggggacacagcagagccctgaggcagtgcaggacattacatggcaag
aaggctgagtattctaatgtgttcatgtctctcttcctgttcttataaaatcatgaatcctactccca
tgataacccattaacctattaatttatgaatggatgaatccattcataagggcagagccctcatgatg
caatcacctcttaaaggcacaatctcccggtgctgccacgttggggattaagtttccaacacatgaaa
tttgggggacacatttaaactatagcaaaattgtaataaaatgttatatagaagcaatgttcttactg
attataattgttatattggtaaagtgttaagtcctctaaccaagggatatatttcagcttattataat
agttttaaatttacaattcaatatgaataacatctggtaaaagttcttttcaagaaatgggaaaatta
gaaatgtttagaagaaaataattcaataaatattaagttcaaactggattcatagtttatgtgaaatt
ctgggaaccaattgcaaggggagaaaatagttacaatagcaatggtgaggatgagaataagagcaggt
atcaacgttaattgagggtgtgttatagttctaatcgtgctatgcccactacatgacttttccctgtg
tgaggtttccgagcttcttcgtagtaatcctaaattgagctggagagaggctagggtaacttactcac
gctcatagagccatagagtagtaaaacctgtatttgaactctggcctgtctgacatcattctgtggtc
ttttaaaccaccactgcttctccatattaaaactccaaatctaggtgaaaagaagaaaactcagaaca
tgttctgcaacaaaatataacaaaatataatgtatataaacacttatacataatatcactaatatctt
tactatgaaaagactctgatacgaacattttacataattcatgcagaagtgttaatcacattgtctgt
gatgagctgtgtatgtatctgataaaattctggcaaccagacatcaactcgtaggcatagatctgtaa
cactaaatatttgcctcgagaaacttaaagaaataaagacaaatgaatgaataggaacatggaactga
gtacaagataaaatcctcctaaagcaatcgatgtacttgctgctgcgttattgttctaagcaaaagaa
gcatggcgaagggagatgtgaagctaaaaacagaatgcttagaaggagatgatagcaggagggaagca
aagatgggaccaagctcccaaaaggcgggctttgaacaaacaaaacagaaagctaagcctttgacgga
tgcacgggatgcaagaaactttagtcaggaaagaggaggcgaagaaaaaccctccaaagaaaaggtga
acaatattttaataggcaaattgacagatagcaagagatatataccatgctatgttttctcattgcag
ctgaagacaaactggggttatttatgctttgaaaaagcgtaaatctaaaaaacaattgtggaggaaga
agcgatgaaaacacgtgttaatacagaaaacatggctccaaggctttaaacttccttgtgagataaat
gcatttacattttccgtagtagctaatatatatatatatacatatatatatatatatctgggaaaata
atacacagtgattttctttctttttttcatctacttatgtgagaaaaaagtaggctatctgaaagctt
ttcagttaaatgaggaagaaagttaggtgatcttgtaaataatatatatgttcaagataatgtaaggc
ccttgtgtagttttcaaaacttatctttaatagcagtttcttctggggatggggtagttcaaagttga
aatgttagaaagatgttaactttttttcctttttacttctccctttcaggatggaattaacaaatttg
attacaaatagatctcagagagaggcaaatgcattgaatccagaagtaacataaaattagatcatgtt
tagttatgcccgaggtcacatggtgataaaaatgaggataaactgaaattgtctgtgagccagattag
tttattttatgccagtcctaggaaaaagacacatcatggtaggatacatcctttttttttttaattat
actttaagttttagggtacatgtgcacagtgtgcaagttagttacatatgtatacctgtgccatgttg
gagtgctgcacccattaactcttcatttaacattaggtatatctcctaatgctgtccctcccccctcc
ccccaccccacaacagttcccagggtgtgatgttccccttcctgtgtccatgtgttctcattgttcca
ttcccacctaagagtgagaacatgcgctgtttggttttttgtccttgcgatagtttactgagaatgat
gtattccagtttcatccatgtccctacaaaggacatgaactcatcattttttctggctgcatagtatt
ccatggtgtatatgtgccacattttcttaatccagtctatcattgttggacatttgggttggttccaa
gtctttgctattgtgaatagagccgcaataaacatatgtgtgcacgtgtctttatagcagcatgattt
atagtcctttgggtatatacccagtaatgggatggctgggtcaaatggtatttctagttctaggcccc
tgaggaatcgccacactgccttccacaatgaacagacacttctcaaaagaagacatttatgcagccaa
aaaacacatgaaaaaatgctcaccatcactggccatcagagacatgcaaatcaaaaccacaatgagat
accatctcacaccagttagaatggcaatcattaaaaagtcaggaaacaacaggtgctggagaggatgg
ggagaaataggaacacttttacactgttggtgggattgtaaactagtacattcttaacatcaatttat
tcctaaaagcaatgttcatagggcacactgtaggccatagatttgcctcacaaatttaaaggcctaag
ccctcaacatgcacagcagtatactcagagactatttgtaaagatgacgattctggaactttttaatg
accccaatcattagcaatgattaaaattaatattcaacattctatatttaccaaggcaataaagtaga
ctaatctattttaaaagggttttaaaatgaagagatgaaacaaaccaaatgattttgatttaaacttc
atgaaaacataagttgcattaatcaggtgattttgttttatgagcattctgattgaagtgatcatatt
tagccccgggagaataagagaaggtaaagtatgggtatggcactgaatttactgagatgattatattg
tttgagttaaagaacttgtattaagaaacaagtatgtgccaaacattgtgctaggagcaagcaatgct
aaaattacatgggtagaaagagagaatgaaatatctagaatgagttagaaacatcagtgttttccaat
gtggagccctgacttcacatgaaaattctcattttcaaacaaggtagtttatgaaaactggactatta
gcaagacagggtgggcatgccatcagtatagtacctggtgtaaaactagaaattttaatcatttgtgc
tttcattttataatcagtaaaatccaaggtaggacaaacttttactttttctgtataatggactgata
tttgaattatacccaactttaattttttgccagaaattatgctttattgtttctctaaaatggtacta
tagatctttatttatttctatatatttatatgatttttacatatatgtgcatttacatgtatatacat
ccataaactatatacatatatacacataaattacaaatatgtgtacctacgtacatatatatgcatat
atcacgcaaatacaggcacattttcaatacccctttttgatttttttccttgaagagcatagcatctg
aatttattatggatttatttttaatttatggtcatgttctttgagtgcttttggtgtttatctggttg
ccccaaactcgctagcattgtaaagaagatgtgcaaagcctgaatctagactgactttcatattgact
ttattagtcaaaaaaagtagatgaaaatgtaacagtccgtgttaaaaatgggaataagacagatgttc
aagccctagcttcagcagtttttagctgagatttactggaagaaaacattttctgaactgtaaaacat
gcaaaatgcctacgtgacagacttcattaacattattaaatgctatgatatagtaaaagaatttgtaa
actgtcaagtgctttgtcaacattaggaatttagttattataggtatttccatatacatgttgtattt
agaattccctttaattttatacttagggttgatttgtattttaactaagtcactttatatatctggtc
ccattatacaagtatacttttccttaggataagaaagtgatctttatatatgtttatcaacccaaatg
cccatcagtgatggactggataaagaaaaggtggcacatacacaccatggaatactatgaatccataa
aaaagaacgagttcatgtcctttgaagggacatggataaagctggaagccatcatcctcagcaaacta
acacaggaatggaaaaacagacaccgcattttctcactcataattgggagttgagcaatgagaacaca
tggacaccgggaggggaacatcacacaccgaggcctgtcgcgaggtggggggcaaggggagggagagc
attaggacaaatacctaatgcatgcggggcttaaaacctaaatgacgggttcataggtgcagcaaacc
actatggcacatgtgtacctatgtaacaaatctgcacgttctgcacatgtttcccagaacttaaaatt
taaaaaactttaaaaaaagaactgtagatactgatccaaaaaaaatgttcattaatgggggttaaatg
attatttctaagtagactactcttgaacccttgaatctttaagaattttctttgctattgaagccatt
caaactctattttattaaagctgtcgttattctagtagattttaaacagtaatacctgaatacattag
aaatatccaaatctgcattacatatggcatctgcagagcagaggagtttggtcatctggactcatgct
aaagtctccgaaaaatccgcttgtcttaatgatggttgactcgctaatgctatgcgtatatagtctta
ttttaagtgattgaatgatgtggctaataacccctctgttagatgcactcagaacctcacctacctgg
gtcctcagctctccagtgaaatctctactttaagtttattttctaacatggtaagagccttcagttta
tgttatgctcaggcccgtcactgtgaataaaatattagaaatggactttttttttttgtattttttta
atggatcccttggaactttaaaaaaattatttatttgagctttctactgttatcacagtgtctcctaa
gcatggcctcccgttttttgttggtaatataattcttacgttattcaaattagtaaccattatttttc
tcatggctagaattctggaaactattaggaaatcactgagcataattgaatggctgtttatttgaaga
gctatgtcaaggcagcatagagttgtattttcttgcaggggctctggagtcaaagagcctgggttcaa
accttggctccaccactttctatctgtggggcattgggcgtgttacatttgtgaaacttttgtttctc
catttgtaaagtgaggtttgggggatgattaaaccagataactcatgtgaaatatttaatggaaatgt
atttggtaggggatttattatttttaaatttggattgcacatgacacatgtcagggatcatgctatgc
attttggatagaaagatggctaagatatcatgcctgactcttaaaaacttacctaatggtaaatgacg
agttaatgggtgcagcacaccaacatggcacatgtatacgtgtgtaactaacctgcatgttgtgcaca
tgaaccctaaaacttaaagtataataaaaaaaaaaacttataatcaactgtagtagaaagagatctga
atggcttgccatttagctaggcacatggtatatgtgcttaattcatactagcagccactacagttgtc
atgattaataatgagcttccaactgcacagaatgcttttaatccatagaaaatcaaatcagaaacaag
tttttgtaaaattaatgtgaaaggagcaacaattaaaatgcaagattgacatttattttctaaattgg
ttctattttctttcacatttacaaaatttataagaaaattctttatttctatgtgatataaagaacta
gaatgtactttgatgtgaattattgttgccagtgctgttcaacttttatccataatttactaagcacc
tacatttagacaaaggcattatccatccctttggggaggatttcagatgattcatacacagacctggt
ctcgaggaatttaagattttctttggggagggaaataaggactttaaccaactcaagagtacttagag
aattttctgaaaataattttatcaatgaaaacttgttatattaaaagaaactgtcattctgacttcca
caaatctaggcttgaaactatggataacgagatattttctattactctcactcacgtcattttcacaa
agtgaaaaggtacattttaactagtgaaagaatagaggaaatggaagtagctcgaggcagtggacgat
gattcaaaaagacagggccctattatttgatcaagttatgcaacgactctgggcctgtttcttcacct
ctggaaggaggaataatctccaagccctttcagactcttttggtaattcacctccagcacatcttcta
aatgccagcattaactgtcctctgatttgtctcatgtttttctagccccatgctctcctgttcgccat
ttaccctcatgcaaggtacaaattacacccatcatcacaagacacttgctcaagtcccattgccccct
tgaagacctgccacacctactctctcaaaaaccatcatttcctgaaagtcctatacagctcatttggt
atttacagtgtactgccacaagccactaagcatcgttttgtgaatacatgacttacagacttagcttg
agtaaagatacttgaaaatgaacaccatttcttggctatcttcctattttgatgtacccttcaggcct
atgaattttagtataatagataaccaataattatttcttggttctttcctgcacatctgaataaccct
atgcaaagtgatagaatgtttttctataaggaggtcctacactggagattgtgtatttcttaatgctg
ttgaaggaagagatgtgtatctaaaataaatagactctaacaaacattaatttatatttctattatct
gttttgtgtattgagatatctcacaaaaataactaaacattttggcattattgatattacatatttgc
catgaatatttgtaaacgaagaaaaatatatatacatcagtaattatcttggcaaactcttcaattat
gcaatattgttacatagattacatatctaagtgaacactggagttttaacaatattgtgtgttcataa
atgttttatttattattgccactaattcttattgccatttcaagaactatgtataagttgttctaaaa
actattaaagtataggtgaccatggtcactactgcctactttggtaaaggccaaatatgtgaagactt
tttaatgtgttaacaaacgttgaaggttttttaacctgttaacaatcagtaggactcttgaaattatt
tcctaagagagtaaattttacaacttgcaaagcatgattaacctcttgtaattataaaccatctcttg
tagttatgtagcattttgttaatgagcaaagaaccattgtggttcctttttacatttcttaaaataat
tctccgtaacctcattgatatctccagtaaatttagataagcttttttttttaaaggagggttaaaat
gacattttaaactaatttttcttgttagttatacagagttgaactatctgagggttttattgacagtc
ataaaaaatttgttattttctgtgaaatatagagaatttaattcattatcatattattaattctgtgg
gccattgtcttaattctagaggcacaagctgttttcatcccactgaaatagaggaatcaaagtatgtt
ccttgctcaaagcacaaaagtgacatactacatagtatgcttcttgagtagtcgtaaatctcatgtgt
taaattacatcccaaagatttcagtatgttttatgactttaataatttatggtaatttctaatctggc
ctttgttgacctgtcttgctttttaaatttttagtttttcgacaaaataattaacatattttaataat
cttccaaaggtgtttaaaatggcattgtatagagatagctgaaggcttttgagcttctgtgttgtaaa
cactttcttaataaaacatgaattgctaccagatgatccagcaatcccactactgggcatttatccaa
agaaaaggaaatcagtatctttgaagagatagctttgttcccatgtttactgcagcacttttcatact
agccatgatatggaatcaacctaaacgtccatcagtggatgaattgaaaagaaaatgtggtatgaaac
agaaattgctgctttaatttatattaaacacactcatattcttctcagctgttaagtattgagttata
gatttaaagaattctattgtgaagactaaagtgactattaaagtaagaaattattttttccattatat
ttaacttatttcatactttaatgttagcgccaatgagcaagactattgaatacaaaaactaattaagt
agtggtgatagtacagtatataagggagaacattcttttagaaaggaacaataacagggagcaataga
aacaatgaatgagtgtaaggtcacttagtgttaaaacagctaaaatatagtacaaataagttgcgttt
taatagtgattttatataattacaccttgatgttttatttgttacaagaattgtccaggaagatttct
ctaaagaccaaaggcactcttcccctaaataactccaaagccagtcctgtgtttctataaaaaaaaaa
aaaaaaaaaaaaaaaaaaaaaaagtgaaaataacgtgatgaacatttttgaggaagtataaaaccaaa
atactccactgcatagctgtttctgcaggtattgtattgatatattacattattcagctttggagtct
ccacatccaatgttacatcatcactctaaattaaacatgtatagataaatgaaataaatgagatagca
tatgaaaatctcatagcccagcccctgcactatttaaaatagaaataccaaagaattgtattcctcat
ctgaaagctatttagtggtggtgtttcaaataaaaattccatctactgctgttgcttccattgtatct
ttttctctgcggtactgaaagagaaagagacccagaaggggccttgtctgaagtgtccctcttttaag
ctgttgctgctttaagcacagggtggacaaatgtaataggagtttcataaaggtggaataaaccagcg
gattacggtgtgggtgaatactttcagatgttaaccaggagctctgcttgcatgctgggagttgccca
tgcctcttctagattgaggcacattatcatgcacaacctaactccaagaaatcttttaaaccactgga
aattgaacccagaacatgtctctaagccagccttttcatcctgacaccgaatcatagcatgagccagt
ctgtcagggatgctgctgctctctaggcaaattttaaatgttgaaataatgaatcatgttttcttgaa
aaccatgtacaccaaagaaaagttagtcattttatagatgatgaatattaacattttcttagacaatc
tgataaattatcagatctcacttttggctctttttaagacagttatgcctcagaaatattaataaacc
cccaagcccttatactgatcagtatgttcactactagctatgagaaattcttgaagttcttgtaatta
ttgtattatttccttactttcattttattagtatgtgaataatatttttaaaaattctagtgtatgtc
ttgtatatattttaacaacatgacttttaattaatgtcttgataacatttcttctagtgtatgttttc
agtaacatgattattaactgtaactttaaaaacctgtggattagatgggaccattttaaaatgtttta
aacctggaaaatctgatggctttaggtttagttcaagctatagatcacctgtggagaatggaactgcc
aaaaaaaaaaatagctgtagcagccctttgagtattctaaaatagggatgttatccagagcattggtt
tctaaagcttccattatttattgatgttgagctttcaggatttagctacaatatttactcaacatcta
agccatgcttttttatcagtcatgttttatatcttttataatcaaactgcttatcactgaaaaaaata
tataagtttctatgtatctggaagaattctctggtgtttcttagatatggattttgatgtgtggaata
agaattcaattcaaggataacagagatgttgtcctgaaaaaaatcgaagaaaatcagcttttctttaa
cattctgtcaaagctcctgactattagtttatcagcactgttttgccaaaggtgtcttctcttctctt
ctttgaaaaaaatcatctgctgctgctacgccgcaagtgtgttcccgctgtgcctgagaagatgtgtg
gcataaaaaaatgggcatggcctgagttaaaagtgctacatttaagccagagctggcttatttattag
ttgtctaatcataggaaaatgacagagcatgcttttctcttgcaatatccgttgctgaaaattaaaca
catgagcagagctttcagagaggttgactggcctctcagacagcacctcataggatggcctgtgttga
agcatctcctttaaccagggtctgtccctcagcattgggttggctcacctagattggattgtcccagc
agaaaaaaaaaacccaaaattcagaatcatatccaaaccggaatactctttcattcacattacttgta
ctaccttttcagaaactggatacctgagtgtgtgagggtaacttagaaacttatctcatggttagaag
ttttagaattagagagcgatgatcatgaaacggacttcatgatcagaagcaatggagcaaggaatgag
atgtctttgaggagtatttccctgaggctgtggataacgctgacgaataatccccaccttaaaagtgg
gttgaccactctagtagctgtaaggtgggagggttctttcttcagagataaatctgtgctcttcactt
gcccatttcccaggttttcatgtaggtagaagaaacacctgtaatctgaagacactcttccttcagct
ttgttagtgacagggatttaaatatgtctttcacacattttccttagatagttaaatttcacttttcc
tgtttgtttttctctgaaggtattctaactcccctcctaatggacttctagagctttctaattctatg
caatttctgttgatttgttctggtaaactttgaaggtaatctctgattcaacttcttggagattctat
catgtcatctctgtttattaactttatgttactcatggtttcttgatgaggactcattaaacataatg
taagtagaaaattattaactacataatatttactacgggttgttatttctgatagtagctagctgtaa
gattccaattgttcttcaaatctttgtctcagtgatctctgtgtagttcttgactacttcaaataact
tcctagaaggatagggatttaataatctcttaataggaacacttaacacactgctggtgggaacgtaa
attagttcggtcgttgaaagcagtgtggtgatttctcaaataacttacaaaagaattaccatttgacc
cagcaatcccattattgggcatatacccagaggaatagaaatcattctaccataaagacatatgcacg
ttgtgtatgttcattgcaacactactcacaatagcaaagacatggattcaacttaaatgcctatcaat
gaacagactgaataaagaaaatgtgctacatatacaccatggaatactatgtggccatgaaaaagaat
gagatcatgtcctttgcagcgacatggatggagccagtggccattatccttagcaaacttatatggaa
acagaaaaccaaatactgcgtgttctcacttataaatggaagctaaatgatgagaacatatggacaca
aagaggggaataacacacactggggcctactggagggtggaacacaagtggagggagaagatcaggaa
aaataattattgggtactatgtttagtacctgcgtgagaaaataatctttacaccaaacccccgcaaa
atgcagttcacctgtatagcaaacctgcacgtgtacccctgaacctaatttaaaagttataaaataaa
cgtatcttattttcagtacaatacaccacagagtagaagggttaaaagagattgcttctgaggaggtg
agatgggggtaaggacagcacaagagcattttggggggtgatgaagctgttctgtgtcttgcctgcga
tgatggctacacgactaagcccttgtcagaactcacagaactttacttcaaaaggagcggattttact
acacatcaattccaataacaaatactttgtctttaagcaaagggatacctaaatatagcgtattgaat
ggatctccagaaaaacacatttttcagttcatgtttcagcctaggcctcatctcatccaggaaacctt
gtcttgcttgcctttacatacatgtggcaatcagtagtttcttttagggctcggactgaacactcaat
gaacttcaatcttagcgcttgtcgtagcagattgacatggtttatttatatgtgtcattctctgtagt
aaaaggaaaggatcaaggccattcacttttgtagtgattgtgcatggcagtatttggcacatagtaga
ttattaattatggaacttctgttttcacacacacacacacacacacacacacacacacttcagagcta
ttttcatttaaatatttgctttagtctccaaagcccctctgcctcaacaccaacccttctatctcatt
attcatcagcttttctcctattacgaaactacttaggaaagcccacttatttagcttatgatggcaaa
aataaatatttgtacttttttttttttttttagtcatcgcttcatagaacagcctctgtcctctgctt
atgccatgtctgaatatatgctggaggtaaaaagagttcctggttgagagcttcaatttgagaaacta
tctgagattactttccaggttccaccgtggaacctgtctgaccttgaacaaatgacctcgaacaagtg
gctgaaatctcttctatttcgtcaactgtaaaatgggggaaaaccatgtctatctcatggggttcatg
tgaaggttaagaaattgcttattcagtgtttagcacagtgcctgatatgcataaagctcctaggaata
ttagctgttattgtatttccttaaagaagcccatagctctatatgccctttcattatatgttttagta
gcccaatttaacatatggataaaatatttttaagttaaatgatttgctaatggattgttgaacgagtg
gcagacacccatattatagacgaaggtcaagtccataacatacagtacatttccccactttcatttcc
cattaccaaaattcattattctcctgagaaactcattatagaattcatgtcagattcatctgtgtgtt
cccagcagtgccttatatccagaaataacactgagtcattgtctagatgtagcagaggtggaatcctc
caaagagaagcctcagagtggccaggtttgccaagtatagggatgccttgattactggccttactctt
tatgctcgtgaattcctaagttttattcctcctgtagtcatagattggcttttaagctacaagctgaa
gagagagaaaacctcttccacctcgttggaatatgtctcttcaatccatttgagccaatttaggacat
gagactgctcttagtctagaaccagtcatcaggagaattccaggtctgattgactcggactagcgggt
caatatcagggcaaaaattccaacgcacaacacgatgtatcagtaaggagaacctcaaaattatttct
taacgtccagatcatgttcctatttttatatatctattttctcacataagtcattaaaatgatgtacc
tgtgcgggtcctttaatgatactcaaagatcttgaattataggctaataactaacttaataagctgca
gaaattaacatttctgctacgtttatgtagcattttcccacatgtacttcagaggcttgagaaaagac
cctgaaataatgactgaataacagctttactcacttaatttcaaatttgttaattcttctgggaaata
ccgtcaacatccattttattatttttctcaattacatgtacgtttctacatcagtggataagttaagg
agaagaattccctcatgataattttttcatgctcgaaaattttgaatcaattttttattttacattat
actctttcctagtcattagaaagggagtggtggttaagataggcaagaatgctttataaggatactac
tctcgtttcaattcttaacatcaaaaaccttaacagtgtgtagactataaaataaaatatctagggat
cagagcattgtgctgaactttgcaggttttttagtcaataatatatatgacgtgttcacagaattctt
tgtcaacaaagtacttttggagctccaggccatttaagttggtttttgtactttttctttttcttcgg
aagactttttttgttctatttacctggaagtgtttcttttttggtactgtgaattaaaatgagaccaa
tctactaggcaggaaaaaaccttaattagattgttgacacagacaaataagaatgtcaattagcatct
actgtcacatgcctctccagactgcttctaggatgagtggcctcaagcagctacatcatctttatact
cctaaagcatcaaggaaacttggagtgacaattcatatcatgaacacatccacagtgatgatgattgt
gcttcttcccccccacccaacaacaaaggatgaatgccaattaatgtattcagttttttgcgtcaaag
gctggatcacttgtgcaatgagggtaatcatcctgaccagacaggccatacaatccatattgtgtgaa
ttaaagataatatgcgtgaaacaccttactctggatgtggttcatagcagtagcaaaaagatgaaaac
tatggtatgctaacattttagagatctgtactctattttaaataattttataaaagtgcatatacaat
aaaaagtgcacgtatcacaagtatatgcctcaaaatctaaagccagtcatgtaatcagcatccacttc
aagaaagaaaacaaaacagtacccctggttcctctttgcaatcattagtctcccaagagtaatcaccg
atctgatctgtgacagcatagattggttttgccctactatatttttgctgaattatacaatatatgct
ctttaatgtctggcttcttagtgcattgtatttgtgtatcagctattctcttgtgtgtagttattaaa
caatcattttatgggctgcataatattccatagggtaaatataacagttttattgataacttagctat
tacaaatagtgctgttgcagacatatattctattacatgtcttttggtataagaatttacacatttca
catgggtgtatacccagaactgagattgctaaatattggggcacattgtatacattttgatttagtag
ataagatattgccagatatcgtaaatgcacagtttgataaatatagagatttatactttttctagaga
aaagccatcaatatcagtgtatgtgtatatatatacgcgtgtgtatatatacgtatatatatacgcgt
gtgtatatatacgtatatatacacacatatatatacgtatatatgtgtatatatatacgtatatatat
acacatatatacatatatgtgtgtgtgtatatatatatatgaaacaactcagaagcagaaagataccc
catgttctcacttataagtgaaagacaaataatgtataaacatgtacacatggacatagagtgtgtag
tgataagcattggagactgaagtgtgggggtgtgcaagggaatcagtgataaattaatggctacaatg
tacataatttgggtgatggatacactaaaaatccaaagttcaccactatccaacatactcacataata
aaattgcacttgtaccccttacattcatacaaataaaaaattatttaaataaaaataaatatgtgtat
atgtatgcatacatacatatgcatatacatatgtgtttgtgtgtgtgtatataacttacacttaaaat
aagcatggatgctgcaatgaatgctcaatttacaagggttgtccatccaaacttgtggcaagtatctc
acctctcaagttgttttcttttttcttcatatatttcttgcttttgtctaggaaggaataatttggct
tgcctttcaagagtgtacagtcagcatgataacccaaacacttaagacacgtgctaacccatgtggat
cccttgagagaaggaaaacagtggtccttttactgggcagatagagcccggggccaggtttcgtggct
tgaagatttcagcttctctgcgcctctcagctcagtgcctctggaagcaatttacaacttgtgaggcc
atactcaaaggccctgttattaattccccgccttccgagaccccatttcagaggatctcaattgctct
cagagtgaatttactgtttcctgaattccgtaatcccaatagcaggtctgttgtcctcattagatagc
ttaagttagagtcggcagtgtaattggcaactgagctactaagtatccaatgcttatgtggaaaatat
gttccctattgcaaacaactgatattcatattcaatttggcaccatcatctatctataaagcagatac
tacttgtgtttattaagttttatcccaaataattattttagtaataatgcttgaaaataggccttggt
catttgcatgtctgtatatggcatatcctgagtctttgtatgtattagaaagatcactcgttttgact
tgatggtttaataaaagatgtccctcactttgggcagagacatttgaaaaaggcactccaaccaggga
cctaagaggtgaatgagatgcagctctgaatcaggtcacacggcctcaggaaggaaacatcttggttt
tcacatccctcacttctcgatgtcatgtgcaatacacaaatgacccctcaacacacacacaggcacat
acacaaacacacactcactcactcactgtattgtctctttccttgactaagtccttcttactaactca
agctctaaagcttttttacttacctaaggtgagtgtgtgaggatttgaggtttcaatattaaaattca
gaaacatttaaagttcattttaaatattagtaaaaaaaaatcttgacaaaatacaattatagacaaaa
agaaaattcagaatatttggaatttaaggttgaggttacagccctatttatgaaatattagaagaaaa
atgctggagagaataaagcaggtttatgagtctgatagaaagcataaccagatgattatgcatatatt
tgcatatgcaaagctttctaggcaatctgaacatttaaacctacaaatgtggctgcgatgaacagcca
cagaagagcaggctagaacagaagaggaggctagaacagaagagcaggcagaagttgtaaatgaaatg
ttaattttcaatggttgatctcccaagtactggaacagatttgtgctgttttcaaggttttggttcaa
agaatccagtagtgtattgaattgttttgtggcacttccctgttattttgctttgtaagctacctcaa
tccatgaagtggctatgagccccttatacaacactgttgatttttttttccttatctacgcaaaagat
ttttgattcagggccaggcatggtggctcacgcctgtaatcccagcactttgggaggccgaggcaggc
ggatcatgaggtcaggagatagagaccatcctggctaacacggtgaaaacccacctctactacaaata
caaaaaatcagccgggcgtagtggcatgtgccggtagtcccagctactcgggaggctgaggcaggaga
atcacttgaacccggtagccgagatcctgccactccactccagcctgggcgacagagccagactccat
ctcaaaaaaagaaaaaaaaaaaagattttttattcaggtggctatcagactcattaaatagaagcctt
aggttaagttcacgggttgctagttggaagcctccatggactatgttcataaaataatagaaaggagt
tatgcaggacttcttgaaatgttatttaaaaagtcagaataggctttctattacttgtctgaggtcaa
atacatgtagtgctttctgaccatttcatccagggtgttagctaggacaataagaggtgcttaaaaat
tattagattgagtaaatgagaaagcccttagaaacataggaacagaatgacccttgctttggatctaa
tattgactcccacgcctaaatccctttggagaactcctttattttctcttccatcaagagcaggtata
aattaaaaacaccattaaaggggccatctagctcagctgaagctttcatcacacatgtaggggaggta
tggttgggagggatctttttatcctttaggtcttcaatttacataggacttttgaataatcaaatagc
cccaaagagctgatcttaggactagttgtaattgagactatttctccatggggtagaaaaatctagtt
gtaggaaaactgagaagtagatgtatgttaacctcaaaggctgttttttacaaaggatgttaaagcat
catctttgctcagaaagggagcaataaaacaaatgagtggaaataacaaaaggaaataatggccaggt
gcagtgcctcacactagtaatcccaacactggggggctgtggtgtaaggatcgcttgaggctagcagt
tcaagaccagcctgagtaaaataggcctcatctctacaaaatagatagatagatagatagatagatag
atagatagatagatagatagccgggcgaggtagtgtgcccctgtagccccagctactcaggaggctga
gatgggagaatcgtttgagcccatgaggtcaagtctatggtgagctgtgctccctcctgccactgcac
tccagcctgggtgacagagtgagatcctgtctcgaaaacaaaaggcatactttttagatgtaatggaa
tagagtacttccaaacctggctgcctgctggagttgtattggaagaggttgcacgacttcagtggaga
tggcctagatgcctgctcagcagtcatctagttaaagcaactaagaacatgtaatatgaaactgcaaa
aagagatcgtgtacgtaaaatcactctgggctcctcagatagagtaataaacacaactcctgacagcc
aaataaaaagagaaataatacagcccttgacttccttggttgctttgacatactaagtaggtgttaca
ggttgggttctctgggaaacagactctaaaacatttttatttttactttatttgttgttattattatt
attattattattttagacagaattttgctctcgttgtccatgttggagtgtaatggcacaatctcgtc
tcactgtaatttccgccttatgggttcaagtgattcttctgcctcaaactcccaagtatctgggatta
caggcaagtactaccacgcctggctaattttgtatttttagtagagacggggtttcatcatgttggtc
aggctggtctcaaacacccgacctcaggtgatccacccacttctgcctcccaaagtgctgggattaca
ggcgtgagccactacgcccggccagactctaaaataaagtttaatatgcagaatacttatcagggaat
gcccactggaccaatacatattcaagagagggcttagaagcaggattggacagaaagagaagttgagc
tgtaatgcaggcccaataacagccttagtgttaagcaggctgagagattcagcagttaatgagacagt
caacccaaacagttttataggcatcaaaagtatgatcagcatggtgtcagtttcctgtgtcacttgtc
ccacagtatgataccaaaattaaagagaccagatgacatgcaacacaagcagtgtgcactctgttgtt
gagaagccaatttcgtcatgcaattaagcagttttatactctgcagctgtactttaaggggagctgag
atggaacatcatatgtctcaccataaccagaaaggcagatgagaaatgttctatcgccacctcccaca
aggtaagggacttccctaaagatacagaggtgggtggaatattgccttggtagacttcctctcaagac
tgcctatcttcccatgttggaaggatcacagagcatttgtcaagacgtgggtcaatctgcagttgaac
tttgtgtatgtggcctatgtggatacttataatatcattgggcacctccatagagctgtttcccaatt
gaccaaacatatgggaagcttcagagcttcgaatgacccttcagagtagtcctgagaacagtgagcct
tactactcctgcattaatcagtcattggatgatagccttctcagaaataagtcatgaccttgtgcaag
ggggctcttcatggctgggaccacccctaaaactgagagctgaaggctgtctgccaccagcccttcca
cctgctgggacaagttctttattgaagggaaatctgagtagttcatcagcgtccatcacagtagtcaa
gccgttcattcttccttcttatgacaacattgtgcttattgttatgtaatccctttccagaacatttt
aggttaagttttaaaaataatgcatataaatagacaattcaaatactggggaaaaaaagcttgcactt
atattgttatagaaatgtgcacacttaaagagctgatttcttctgggtatttacataactttatttaa
aaatccatccatttttaattagctgtttttaatatgcagttagctaagatattataagccatatatta
ggctaatggacatttaacagcttagttaagttcttttaatggaaatgctgacaaacctttgtctgtaa
ttatagcaacactgtgattacagaaggaggtgcctctccttgttgtttgcagccctaaaattccatgt
ggctataagtaacaaagtccattattagataaacacaagtcatacttggcattacttgcattactcgt
ctccttgctttatttgaatcattttttaaagttgtaaaatgtttttcaaaactcagaatagtggccag
ttaataatatgattcctcttatattatgagattttaaaaaatagttcaccagtttctggtggcctcta
tacccattggcaagtcctagccattgtgaattaagtaaacaattctttatggaaattttttaatcctt
aaaccctataagtttttattcatcatgtcaggtcacttgtcaaagggtttaacattcagaattcaaca
aaagtttatcaaacacctattacaggacgtgcaattttgggcgcactgggatttcagcaattaacaat
caagatatgatttgtatcgacatggatattacattctctcacaggagacagaaaacaaaataactaga
aaatatacataaagagactttaaaatggggtaaaattacagattgtgacaggatgaccactttggttc
agaatatctaggacatttttttctttttttttcccctccctccctctttcttttttttctttttcttt
tggtgcaatctcagctcactgcaacctctgcctcccatgttcaagcttttcgtgtgcctccgcctccc
aaataactgggactagaggcatgcaccaccaggcccagctgatttttgtatttttagtagagatgggg
tttgaccatgttgcccaggctggtctcaaacttctgacctcaagcgatccacccgcctcagcctccca
aagtgctgggatttacaggcgtgacccaccaggcccaagcaaggacatttttttctgagccatgttat
ttaaacagagatctgaatgacaagaaggggccagctctgtgatgtaggggaagaaaaatatgttcctt
ctacccttctaggctgcccagctggagtcctacaaagttagagtgacaaaagacagattaacaagagg
aaaagcctagaagtttattaaaatattcagtgcacatacacctggtagaaactcagtgatgagtaact
caaaggggtggttagaatgttgggtttatatagcatctgaacaaagaacagtaaacttgtagagaaat
gacaaaacaaagaaaaaaggggtttaggtatttagggttgccaaactgtaggaaggtaaatatatggg
agaaacatggagtatagtttgtttatgccaagtctatcttgagatcaacttttcgtattcttcatggc
cataacaatttcccaggagagagggcttatagcagttatcatttctcagaagtttctgcttttattta
gacaagggaagcactgggaaggcttctttttgcttatattgattcttacttgcctctaactaaaagta
atctttatgtcaaagtgccatattttggagtggtatatattgatctcctataataacaatcaaaagga
acagtattctaggcaggagtaccactaatgcatagtgtttggtgtaaagacaagttaacatattcatg
gggcaacaacaacaataagccaatatggctaagacattgaggatgagtgagttggagaagtaggcaat
ggccagctcatataaagacttgttcgtttttataaattgtttagattttattgtaattatggtggcaa
gtgattggagagtattagcttcactttgactggcttatcgaaaacggaatgtagggggtgaaagtgga
ataaaaagaccagtcattaattgagtagtccgtgtgagagatgatagtggcttggacaaggacgattg
tactggagagattgaagcgactgatttcagatttgtagtcaacaaggcttaattggtaggagaaaaaa
ataaatcagtgttaactctttaatgtttaacttgaataattatgatgagggtattaccatttattgag
atgtagaatattataaagtaagagcagatttgttcaaaaagtatcaagaatctttatttggacatgct
agtttggggatgcttattagagaccctaggaaactgaatataaatgtggattttagagaagagcttag
ggctggcagatgcacattaaggatctgtctagagccatggcgctagagacctccaggagaacataaat
agtctcaagatcaagccctgagacactcagatgtttagaagtggaacagaagagggacatccaatata
gaataccaagaattaggaggggaatcaagagagtgtggcaatatgaaagatacaaaaagagtgttgaa
gggagggagtaattaataaccagcatgttatgaggggctcagtataatgaaaagataagtgactattg
gatttcgcaacatataattttttggtgatctggacaagagcaatttgaacagaatgatggatatggaa
ggtccagaggagtaggctgagtaaataatataaggtgggaaaatagatacaaagattatagacaactt
tttcaagaagttttactgtgaaggggcacagcaagctgagacagtgaggataaataatagactcaagg
atggtaactttagaataagaaatttcaatctgatgggatttaagtgttagcaaggaagctttaagaag
ttattttccccattagaatgatctgaaaaatgttttagaacattcctcttatattctattttatcaca
tttatataactttcagagaattgaaagaggtattaagttattatgaaattttctgagattaataagat
aacaattataggatgttttcttttagttgaaatacacctactcagcctaatttttataacttcttact
gaagtataatatacttcagtagaaaagcatgcctaatataaaggtgcagctagatgaatttgcacaaa
ctgaacacatccctttaaccagcacttagattaaaaacagaaccttgatgatacctcagaggccccct
tctgccccttttcagtctctccgtgctacccccatggataagcattatcgtgatttctaataccatag
attaattttgccagtttttgaattttatgcaaatggatctatttcacctaattgtaaatatataacat
tgtcatagcaaggcactcattgccttacactgaaaattacattgactctttgccacaagcttagactt
gctttctcattttattatcatcaagcctatagctttcacactataccttgttcctgctcttccctact
ctatttcttggtagatattctatatcagtcttagagtgcagtttgcagaacccctccatcagaatctc
ctagggagcttgttaataatgcagattcctaggcccctcccatggtttatgaatctgagagtgaggca
gacaagactataccctctcatgcctctataatgtaataatgtcttcctagaatgttctttgctgcatc
tcttattaaagaaatcttatgggccgggcagggtggctcacgcctgtaatcccagcactttgggagcc
tgaggcgggcggatcacatggtcaagagatcgagaccatcctggctaacacggtgaaaccccatctct
actaaaaatataaaaaattagccgggcgtgctggcaggcgcctgtagtcccagctactcgggaggctg
aggcaggaaaatggtgtgaacccgggaggtggagcttgcagtgagctgagatcacgacactccactcc
agcctgggtgacagagcgagactctgtctcaaaaaaaaaaaaaagaaagaaagaaaaaaagaagtctt
atgtttcctttatggccagagcacaacattgtcatgaagtcatctaaaatttcccactagaggtaaca
tctccttcccctgtctagctcttttaaagcattacctccatttgccttgtatcatagctgcttgtaca
cctgtctgtctttccgctgaggttataatcctctggagggtcatgactttgcattcctttgtgtctcc
cattagcagccagcacagtgccttgcatactgttagttctaaataacttctctctctctctctctctc
tttttttttttttttttttttttttttttttttttgagacagagtctcgttctgtcacccaggctgga
gtgcagtgcaatggcatggtcacagctcactgcaacctccccatcgtgggctcaaatgattctcctgc
ctctgtcttccagtagctgggattataagtgtctgccaccacgcctggctaatttttgtatctttagt
ggagacggggtttcaccatgttgcccaggctggtctcgaactcctggtctcaagcagtctgccctact
cggcctcccaaagtgctgagattacaggcgtcagctgctgcgcgcatccctaaataaacttttttttt
tttggcatgaaatctgtaacactggaaagatgttattgccttagaataattaagagattaaatgtaga
atctcaaaaacattcatttttttccatgaaaactttaccaggcctcaagggataggaaaattatgggt
acagaattgagaatctgtaggaacttgcaagataaacaacggtttcacaagaaagaccttgttggaga
gttaaattttcagacagttgtaataacttcacattaaagttttgtcaaaaaataagtatctgcatgtt
ttgtttgccttccaatgccctcattttatttgattttttcccataagtaactatagtgaaagcacgaa
aatgtgtttctgtgtttgtgtgcctgtatgttaattgtgactgtttctattgcattgttattgcagaa
cctaggcacgcactctgtaggcttgggtgctttctccaactgaaaaaaatcctacatatggataaatt
atttttacagccagtctttaattttacaagtggtccccctccttctgtttttaggatggcagagagaa
tacatatttacttaccattatcacttactcatgctttgagcttgaaggaaatgagacagaaaaatgaa
gtaacattaacttctctctggaactatgtttctcatattagagctttatctgaggagttcacttcctc
tctcttcaatgctttgttcctctccagtcgattcaaatgtcctcttaaagcagaagttccgaacctct
ttctgtgacttcaggagagcatgagaatgtaaatataagttttaggactaaattttcaaagacttttt
ccactcagctctcttttcctcttcggtttgttgttgtcgttgttgttgttgtcgttgttgttgttgct
gctgctgctgctgtttttccccttccacttccgtaactgagctcttagggtccatctggaatctgatt
gcaattaaaaaaaaaaaagtttatttttacctccttgtacgtgctttctcctaaagcaggagtcagaa
gccttttttctttgaagggctagttagtaaatattttaggcttgtcgtctttgtcgcaattactcaac
tacgctgttgtagtatgaaagcagacaatacatacctgaatgagcatggttttgttcctagcaaactt
tacgcacagagaaatttggatatcgtataatttttatgtgttgcaaagttgtattattcttttgattt
ctccccaaccatttaatatgtaaatcccattcttagcttgtgtgccatacgcacacaggcagcaaatg
cgagttgtcacacaggctatagtttctgactttatgtcttaaagtaaacagtaataatcattctcttt
ttccaaacagtccactaatctccctttgtattcagcccttgcatagtaaacgccgtttcttcatcatc
ctgatttttattctgagaaaatactgtatattgttcccatgcactagggttcggggaaatttaaaagg
atgtaggatctccttttcattggtcctaaaattgcactggggaggcaggtcatgtttatgaacagata
aatagtatcataatataatcatgcatttctatggctagcatttagaactatagcttttgatgtcatgt
ggtttttatatggttgattatttttttcttatttataaaatgaaaaagtttgagaatttttcatctcc
ttaatgtattcccttatttgagggaaaagtatttacctactacataggaatttatcttaaaattttct
ttgtctatctatttttatggaatataatcgagcaactattttactaattaatactttaatatcattat
gaaaatgttctcatatttttaaccttataagatcagataattgctatgccaatctatggttgaaatgg
gttcttatacttaacgctatgctctttcttctgagatgtaaaaatatgtttaaatcagaatttatata
ggtgtcaattcaaaatgacagtagttcattattttgattagtataaatgttcacaactaattctattc
tcttatctattaagtcaccaaataaagtatatttgttttaaatatttaacagtttaaattattctttg
aaaacttatgagtctaaagtaagaacaattaacccattcattttgcaagtgggatagttgaattttac
ttgcaatccagggatttttgacagtttgaaatatacatacataccatgtatgtttaggaaaacattta
aaaagagggggttgtaaaataataatagttcttccatgattttttagccataatgtttataatataaa
atatgtatactcttgttattgaatgtagtatgtttctaatttaccagaaggcaagagaataatcctgg
agaatttctcaaggcatcttcgaactctttgatttattgctcacatatagtaatttgccaaatgacgc
cctagtgaactgaaagaattaatgccccgtcctaagtcactttcaccgagggactgaaaacctgcagc
attttgccaattagaggaggaaacaatctaccttgcagagtcaggagtactggataaaggagctaaga
tgaagtagtaagtacgtttttgcaacatacgaatttagcagactggccttgtgtttatttttggccgg
aaccattacacttatttccaaccctctcctttatttgttggttgataatgggctaattttgaatcttt
actgtcaaaagaacattaagagaagcagccctgcctgcatcgcaggctatgtctgtcctttgccgagt
attaaacactaaaaaaaaattaagaaaatactaacaaaatgacaaagcattaagaaaataaaactaga
tgttaaaggaaatgagaaaataggaaaggatgctgtacctggagtgattttttttccccaggctacct
aagatgatcaaaaaagagctaatttctcttaggtttctattaaggaattactagaatatcgggcacac
caggaaactttatcagtggacctgtcctgaaccaaattttcttaatgtatatatgataatttgttacc
acatcccagattattttacaggaattaaaatatatttgaaacactgacagggaaaattgggtaagaca
ttgatagatactacaatctgtacttgaaactgcactcaaggaattcgttagtcaagaaagaacacaat
gactgtgggcccctctgggttttggaacctcttttgtaaagcatttttttttttcccaaatagaagat
attatttttgaaaaggttaaataaaaaatctttgttcactatatagtttcctcctaaggagtaaatta
atttatataaaatattgcaatataaataacaattttaaaatctcaaaagagcagtgttttaaaaataa
tgtagaaacattaagaaatgacttcaaatgataagaatgtcattggagagcaaagggtttttaatatt
acatatcgtggcacgtatatcagcacccaaccgctcaagatacagagttctttacaaaaatcaaacag
aaggaaatgtgccaccttgttcataaactatatttaataataagccaggcagataaagtcactttcac
aaataatgagcaagcccatggtaatataattcatttacaataagatttatctcatggaattcttagac
tgtgctttgaaatttaaataattctgataaatgccaacagaatagagaaatcaattccagagcaatta
ctaacacgttgcattacctttctaacattaatatttctcttcatacatatcattgaagagaaaatgag
gatggaaaataaaaagatcaggtaatatatttgctttctcatctagggttgttatgatcttcaagatg
aagttttattttttactcctagcaaatgatattcttttttattttagtttttattattttatttttct
gtaaattattggggtacaggtggtatttggttacatgagtaagttcttttttttgatatttctgagat
tttttttttattctactttaagttttagggtacatgtgcacaacgtgcaggtttgttacgtatgtata
catgtgccatgttggtgtgctgcacccattaactcgtcatttagcattaggtatatctcctaatgcta
tccctcccccctccccccaccccacaacaggccccggtgtgtgatgttccccttcctgtgtccatgtg
ttctcattgttcaattcccacctatgagcgagaacatgcggtgtttggttttttgtccttgcgatagt
ttgctgagaaaaccacgaggtaccatctcacgccagttagaatggcgatcattaaaaatcaggaaaca
acaggtgctggtgaggatgtggagaaacaggaacacttttacactgttggtgggactgtaaactagtt
caaccattgtggaagtcagtgtggcgattcctcaggcatctagaactagaattaccatttgacccagc
catcccattactgggtatatacccaaaggattataaatcatgctgctgtaaagacacatgcacatgta
tgtttattgcggcactattcacaatagcaaagacttggaaccaacccaaatgtccgacaatgatagac
tggattaagaaaatgtggcacatatacaccatggaatactgtgcagccataaaaaaggatgagttcac
gtcctttgtagggacatggatgaagctggaaaccatcattctcagcaaactattgcaatgagtaagtt
ctttagtggtaatttgtgagatcctggtgcacccatcacacgagtagtatacactgcaccatatatgt
tatcttttgtccctcggcaccccttttctaccccccaagtctccaaagcccattgtatcattcttatg
cctttgcatcctcatagcttagctcccacgtatcagtgagaacatatgctgtttggttttccattcct
gagttacttcacttacaatgatagtctccaatcgcatccaggtcattgcaaatgctgttaattcattc
ctttttatggctgagtagtattcatatatatatatatagacacacgtacatacatatgtatatatacc
gcagtttctttatctacttgtcgattgatgggcatttgggttgatacttgcacacacatgtttatagc
agcataattcacaattgcaagtgatattctcaggaagcatgatgtaagtgacagagacttactttgta
gactgcactcattcacttgttctctgaatgtgctctaggcagcctgagtttctactatgtcagtgtta
catagatgagaaaccccatgggtggtttccacagaggctgcaatactatttttgataccaaaaatctg
tttggttttgtgagccccagatgcccatatggaaaactgaagtgttgatacctctttgtagccctctg
atgaactgcatggttcaccttcctcagcagtttgagcggggtggggagagcgcctgcttcctagccat
ccgattggcctgaatcatcaaaaatgctatcatgaaacaggttctgtttatctgctccagattacacc
catcatgttctagagtgctggtttcatgcttgaatctagatcaagcctgctttcctcccctgcctgta
ctccctgtggctacctacagtcctgctgctgacagataatctaaaccaatagcacctaattagcctat
acgttgctgatggtttaatttctggaatgcaggtaatgaatgtgtttttgcttatccaagtcttccca
tcagatgtcaaatatagaagaacagtgttcagaggtcctaaatttaaattggagtgagaaattcacag
cgcccctgaactcaggcaaaatgcactctgacaagtcaaccagatattcacagatggtctggaggatt
tgaagcctaatttggtgaaataaaattaaatgagtgaaattgtatgcagtcattaatctatcaccata
cttaaaatgcttcattgaaatttcttttactgcttcaaatgaaaaaagatcaaactatgttatagaaa
agcattcaaaacccttacataacatagataaaacttggttggagacttacagaactttctctgctgct
tcgagaaagttacagtgcccacaaatctattgctattagaatattttattgtattcaacactcaattc
taccataattatgtatatgagaaaaatatttttacctataaaataattattattaccttttaaaaatc
tgacattcttccttttttctaaagaaacatatttagatttagcttttattttatttttgtgttgatac
atagagattgtacatatttctaagattctagtgatattttgatacaagcgtataatgtgtaatgatca
aatcagggtaattgggatatccaccatctgaaacacttatcatttcttcttttcaatgccatcatacc
aaaaggaagtaaatagaatttcaaatataaggacagccatgattttacatacatgcctacgattccac
cacaaaccataattacgtcccccaaacttttaacatttcagatactttgtcccaggtatttcatgata
aggattgggctatgactctgttacagaagggccaaatgactaaaatgtctctgaacaatattgattgc
aaatattctacccagttgtcaggtcaatatgttccaattcggaatttataacattgtatctctactcc
caaaccatccaatctcacctacctcacttccatattatggtgggtgatctcagattatatttaagctc
atggttacttgtcaagtagatatggagtttagcctaacttttgaaatttatgctgagattacccttct
cattatagaattaagtaggcagtttccaagtttagatttagcaggcagtttttttcaaatcacttaaa
agttatatttttttagggcattgaacaggtttgaaatcctaccaagatgtcatgtacacatagaccaa
tagaacagaatagagaacacataaataaaactgcacagctacagccaactgttcgtcgacaaagtcaa
caaaaaaataagcattgggaaatggattaaagatttaaatgtaagacttcaagctataagaatcctag
aataaaatctgggaaataccattctggacattggcttgggaaagaatttttgactaagtccttaaaag
caattgcaaaaaaaaaaaaaaaaaaaaaatgacaagcaaggacttactaaaataaagagcttctgcat
ggcaaaataaatgatcaacagagtaaacagacaaacaccaaatgggagaaaacttttgcaagttatgc
atctgacggtggtgtaatatccagaatctatgaggaacctaaacaattgaacaaacaaaaatcataaa
acatcatttaaaaaatgggcaaaagacatgaacagacatttctcaaaagaagatatacacgcagccaa
taaacatgaaaaatgcgtcacatcactcatcatcagagaaatgcaaatcaaaaccgcaaggagatacc
atctcacacccgtcagactggctttgttaaaaagtcaaaagacacccaatgctggcaaggccgcagag
acaaggggatgcttatacactgttgttgggaatgttaattagttcagccactgtagaaagcagtttgg
acatttctcaaagaacttaaaatagaactatcatttgacccatcaatcccattactgagtagatatcc
aaaagaaaacaaatggttctaccaaaaagacacatgcactcacatgtttgtcacagcactatgcacaa
tagcaaagtaatgggatcaacataggtgtccgtcaacgttggattggataaagtaaatgttgtacaca
tacaccataaaatactatacagccacgaaaagaagaaaatcatatcctttgcagcaacatagatgcag
ctagaggccattatcctaagcaaattaacataagaacagaaaaccaaatactatatgtactcagttat
gagttggagctaaatgttaggtacttatagaattgaagatggcaacagtagaaactagggactaatag
aaggggaaaggaaagggggagacaagggttgaaaagctgcctattgtgtactatgcttactacctggt
taatgggatcatttgtatcccaaacctcagcatcacgccatatatccaggtaacaaacctgaacatgt
accctctggatcttaaaagttgaaaaaaaaagatgtcatataaatattcgtggtcactaaaagtatct
aatgtattatacataaaaataaaaattgggtgaattggaagtgtattctttgtatcaagtcatgtcgg
agatcctattctgctttgatcacagtgtgaattcttttgcatttttgttaccagtcacttctttattt
attgaactaataattacatattctgataatctgtcagaaagataaaaacattctttgtccatgtgtct
gaaaatttttaacctatttttctaatgttttaagtgagaagagcatgttaatactgaaattgtaagca
gtagactgaaaaatcatcccaatccatgggttatatattgaattgcttttaactgtattactaaatat
taagcttaatttattttatttctacatatccccatttccactataggtgatttgtatgaatttaggaa
cttccttctctcatccatttttatattaaaactcagactttctaaaacaatatttctatccatccatc
gttggtaactatgtactgacatgttttgtgcatccgaaaaatgttagcattagtttgtgcgcacagaa
gtaattccagtcaccatatgatgagctgatttatttatttcgtaagtgtgttcattattattatctct
tcagcacccaaatatataggggacttaatgatacctacaagtaaaaacggaagacaaaaacgccctgc
tctctacagaggttaaaatgtttttgcaacagggctctagatctcagctgtgaaagtagggacgagat
gaggctaggcatgcagtgtcagtataatacaatataatcaacatgtcagcatctaatgcaggtgttgc
aaaacaaaatgtacacatgggtagtcaggtaacagaaaagcatgaagtagtaagggctatctatgcaa
gaggttccaagctgactatatactgaaatatttaaacactatgtggggcaaataaaatggacattaga
acagttcgatggtcagttggggacttctgctctttcttccagtctctgaacatatcttaaagccacaa
tcatctatttttatttattgttatacatttatttataagccagcacccctgtgatttaagttctgttg
aaatgctgagttggaaaagatcgatggatgggggaaatttagtgcagaggttttgccccaggttcaaa
atcctttataaaatattaatacatggaacaaatattgaacaattaaaccactgataagttaatcaatc
tgattcaaagtacacctgtgaagagggacatggcaagaaaaatattacagtaagaactagaaacattc
cttcatggctgcttgatatggatatgtcatgtttaagaaaattcttctttagactgttgagatttttt
ttcctgacaaagaagattcactgtcgaggaaagaaagaggtactgtgaaatttgttattgaaaacatg
cacatacttttgtcagaatgagttaaagagtgaacaaaatgtgcctattacttacgtgttgtgctgtt
ttaattcaagattaaaatatttaacgtccacagacaagaccacttttatatgaatattatttttctgc
tttattgctcaattttattaccatttcaaaacacccgtgttgctttctatggccaaagatgtttagca
cttttcatggttatacttctgtacagtccaaaatacaacacttactttacacatacacaaacatccaa
tgtattttgttttctgtcaagtaaagacaatgtctgtgttattaagttaaatgtcactttcaaataca
ggatatgttgatattagaatgttcaactttatttcctcatttaagcaaattacagtgtgaagaatgta
actgcagcaatttataaaaatcatatcacattcaattatgagagcaaacttgttttgtagacttgaac
tagtttcaattaatcttggagttatcatttcaaaaattctaaacagagagaaatacggagtgtaataa
tggtaggtctttgggtaagctgcttccaggaaaagaaagcaattatatatgttcacatagcactgaca
aggagaaacaaaactttggacggcaaagaacttgcattagtctttttgacatgttcctgtggtgtgat
ttattacgtagacaatcagctcaacttctcaagtttgatatccttggaatcatttgaaatttaaattt
taatgaaaattcattaattccaaggccaaaagaagtgattctaattgcttttgagaatcagactatga
aagaattctttggcaaacttgcactgtcttttctcttttatcattggttgcttcgtaggtacttaatt
gaaggtcctctgattatcagcacgggctgacatcagttcactccatgcattttaaacagtaggccaga
tgtttaaaggatcagctgaagcatcgatagcatgctagggtgaataataaaattttcattatctacaa
gaagcaaataaaaagcataagcattttcccccattatcctgaaggagaagatgaatgcctaagcaaca
ttttaagaatgggttgagtgtggcctgtgggaaaatttgggtagaaaacttgtagttagctaatgtat
atactgtttgcctctttagctcaccatatacccacacacatgggcatgcatgcatacagacagacaca
tacaatacacacaacaaacaggaaattcagatatactgaagaaatgtatttaagggattactaagttt
ttgtaaataaaatcctttaagatgctgagaaacaatggaagagaagtaggacatgatggctcatactt
tcgtaatttacttgtttaacgtttgccaaggtttaaattaatgtagatgtttttgtggctaggattaa
tgatctaacagtttggaataattaggcacttttatcacctagaaagcccagaaacccagcatgcaaaa
attctggtatgtctgcattttacacttagatataacagagaaatgacaagtagtcaagtggatagaga
aacgaatgattcttcacacatgcacacacacatagaaattgtctttttaatagtattttaatgtaaca
catttatgcataatttctccatagtgtttatcttatagtgaatatgtgatgaatagtctctaacatta
gtggttttatagattaaacataattaaggctttatatattaaagagtcaattggtgacattctaatat
aaacatgtttatctcatatacattgaaatattagataattcattcgttgagaataaatcgaatgagtc
aaaacttttaacctccactttgagctttgtaatagtatccactgaaaatattcatgaaaatttttaag
tcatttctatttatatattcagtccaaacatctcacaagtttaaaatgtaaactcaagaatataattt
ctgtattctacaattggaagcatccatcatatcagatgaacttatatagtttgtgaaattttgcaaac
tttctgtttagtaaatcttaatgtcaaacattttaacttccaggttgtctttcttttcagttttaata
tccgcgatctttgtatactcgttgaatggattctcaataagtaacccacaaatatatatacatactat
gtacctacaaaaaataataaaaagtaaagaaatcgacacttatccatacctgtcccatagtaataaac
tattcataagtatatttgaaagatatgagaatcataaaagttcgtgtttgcacccttttgtgcgtgga
atcctaggtttgcattttgtggatctagactttttggagtgtggaaataaatgaaacaaataatcgag
acccagtcttatattcaggttatcattttactacataaagcataaataacatttgcagtttgtttcta
tggctagctctaaagtcttagcaacgagaacattatagaaagacttcaactgtagcttccagcagaac
ttctgaggttccgtttatggactaagcagcagttgagggggacaaaactcataggcaattgatcactc
caaaggatagattgtcttttctaacctaatcaaaagatttatagtgaaggcatattcagattttgttg
aaggatatggatatataatcatgtgtgtgtgtgtgtgtgtgtgtgtgttagacatacttaaaacatta
tttgagtagaaaattctgcacaaatggaaaagtataacatgtgttatatccacacatgttgagcattt
acctggctgaaacatcaaaagctgaattgacttaattgaatgttgaatacttaatagttactttgtag
tgactcactattaaaacattatctcaagctttgtcagaattaatttttttaaaaaactcagattagtg
tcaggtttactgaaacagcagatctgaaattactgtgtttttttttcctttcaataatcagtttctaa
tccaaaattgaatatcagttccaactctacattcagtttctgttttacttgtttggactggcttttgg
ttctgttttccacatagatcctctctgtgtaagacaaagccatttgtgcagattaaattttactgagc
gtgttaacctatttaaaacattcatccaaaaagactagtatgaattcttcatatggcaagctgcttgt
tttaaaacttccatttattctaaaatcctttttacttatactttttaagaaacgtattcccgatatac
aaaagtaacacatgctcattaaaacaaattaaaaatagtattgtataaagagctgatacatttctgcc
ttgccccatttaactttcttaagtgttcatgtgaatcatccattcacatcaagacatttatctgtatt
catatgaacgtgttttaatatatataacatatatagaattttatataaactttccttttaaaatagaa
atgaaattatatgatatatttattctgtgtctagctcttgtcacgtaattattcaagaacatatttct
aggttaatatctgtattcttaggtagcattcactaactcctcatctacttgttttcttccattctaat
tgtgtttaacatttcttcatacaattggttgtcatttggtcttcrttcatggagggtgcataatgttc
attctcaccaattctttacactttacataactgcttgatacgaagccagaccttataaatatcaacaa
agcaggaacactgtaatcagctatcagtttcagttgagctgaatgaccctgaatatgtgtacacatat
tttccaggagattttaaaactgacacctcagatttctaagacctggagaaatcagcatgagaaacatt
gatctatattattccgtgaaatgatttcactaaatagtgaagcatctcccacatgtggactctgtaat
ttattagaataaagagttcatgtgcttctgaagaacttgaactactcttctggcctccgtacattggt
ttcttagctataggaaggctgagcatgtttttcctatgcgtttcctttctagctcatcattttagtga
caaaacaatctttcgtggtgttgctctagctatagaattgtttcagattcatttgaccaaaggtggca
aatacaacagtcccaacaaaaacaaaagacctattacagaatgatggaaatgaccccagggaacaatg
gcacctccacatttcttaattccaaggttataagcagtggtgtggacaattctcaattccaatgctga
atcgccttctaatttcaaatacctgtgctaaaaattatttacgtctactgaaataatgaactggaccc
caccaggaatggccgatatgcttgtagtcagagcacaactgtagaaagaaaataacattttaatttat
agaggtatgatgatagctgtttcatactgttttcagaacgatgaatggcctgctcagtagtttcttgt
catcgtactgagacactttaatttcttaccagctgagatgaggaatacgagcccagtgtgcaggtgaa
attgcttaacaggagccattaaaatttggaagagtcagaatagcatcaatcaaaatgctttcagtgta
ggaagtaaacatgtactagcctgacccacctgtcttttcttttaggtatgttggtaatattacaatca
ttttgaggtatccataaacaactgcttagatctgaagaattgtatatctttctttactctgccctggc
ctggggttatggttctcattgagctctaacctttcagaaaaaaaatgtagagaagtggttcaagaaga
atgctttatcttgcttcataaaaatgatagtgatagttttattgaaggcttactatgtgccaggccaa
agtgcgttttattatcgttcccattttccaggcaaagaagctggagcacagagaggctaagtgagttg
tccaggatggctcagctaacatgctgcagttgggatttgcacccagaccaacttcttttcaaccactg
tcccatcctgtgtcttctctactcaaaaagtgtttcagctccaaacctgaaactttaaagaaaaggaa
atccttagtggaaagactaggttttagtcacaaattatctccttccttacattatttgtctctttttc
aaatactccaagctttgattaaaactgtctatcactaggaacattgtagaattgctaaggtggaattg
ttaaaagaactcaattccaattaactttgccattgattactgtgtgttctggaggggtgttctttctt
tcaggttaatgatgctttattgtatatctcaaagattaaaaataacaatgaaggaagtagcaaaccgg
aacttctctcacaatgcatctttcaatctcgtgctttaaatgaagataaaatcatggctgtggtaagg
ttgcaggaaggatgatatagattaagtttcttgcaaactgccctctgaattttcaatagctgtagaag
gtattggttttccaaaaaattgacaaattgaggattcattcagcagtttttttctaggtctcttacca
gaaagtgatcactaaaaagtgtagggaaaccactcaaagttggatagatcattattttcacttaagca
ttttaatttcttgaaggagctttataatgcaacaaagaatttacagtcctgtgtcaccgcttaaattt
tctagggtcatcagtaaactcagtggaaataaattagttcatgaatataattgacccttaaattctgt
cactgtgcaagtaatcggtgggtctgctggatatggctttcgagcagacaggtcaacttcttcaaaca
gagaagaagcatagcataaattgaagacaaataacaaactacttgtttcctccttctttggcatcacc
ctatggatggagtatgcatttataatttaacacaatcaagagatctttattatcctacttttgggtac
aactgcttcgtttctcttttgaatctctacagctatttaaaaatctgttttgtaaaattctttaaaaa
actaaaacatcagattcatatttcaggtatcttactatcttataccaacttaagcatccagtattatc
acccacccttcccctgagtgaatccttagcactgggctcttcctgttttatccctgtgcatgctgagc
tctttctggccttcaagtctacttccgttgcaactgttgtctgaatggtctctctatgtccttcttac
tctctaaatatttcggaatttaaagcctggaataatctaccttagtccaaaagatatgctacactatt
ctagttcacaatgatctcacactgccgttgatacacaacatttaatatcaacttaatatctatttcag
ttcattacgaggtcacttatgctacatcttatattgttgccttggacttttattatctcttcatatat
gtgtttatggtgctcccaccctcacgagaagttgcaaataccatgttagctgtctgatggctttctat
gttgtcaggtataccatttcccaaccagttggcattcaatgattaagttcattaacaaagaattgtat
gtgttgaaaaagatgtttttttcttaatgaagcacttgtttttatttttttaatgaaatccaccctct
taataaattttaagtgcacaatacagtattgttaaatataagcaaaatgttgcatagcagatctttat
aatttttttaaccctacatgcctgatagtctatacccattgcacagcatctcaccatttcttccctcc
tccagcccttagcaaccaccattgtactttctgtttctataattttgactactttagatacctcatgt
aagtggatgcgtgcagtatttgtccttttacgacttgcttattttatttagcaaaatggctacaagat
tcatccacattgtagcatatggtaagatttcctttttgtggcagaatgatattccattgtatgtatat
aacatagctttatacattcccctgtcaatagacatttagtttgttcacacctcttggctactgtaaaa
atgctacaataaacatgggaatgcagatatctcttcaagatcctaaattgaattcgtttagataaata
tccagatgcgggattgctagatcttatggtagttatattttttatttttttgaggaaactccatattg
ttttccacaaaagctgcacaattttatatttccaccagcagtctacatctccaattttcctacacctt
caccaacacatgtaatgatcttgggctttttttttttttttttttaataatggttatcctaatccgtg
aggtagtatatcattgtggatttgatttgcatttccctggtagttagtgatgttgaacatcttttcat
ataactgttggtcattttaatgtcttctttggagaaatatctattcaattcctttgttcactttaaaa
attgggttgttcgaatttttgttgttgttgttattacgttcctcatgtattttagatattgacacctt
atcagatatatggtttgcaaaccttttctctcattctataggttgcttttaattctgttgattgtttc
ccttgctttgtagaagctttttagtttgatatatttctgcttatctagttttgtttttgttggctgtc
cttttagcgtcatatccaaaaaaaattattgtgaagaccaatgtcaggaaatttttcccttatgtttt
cttctatgagtttcatagtttcagatcttatttttaagtctttactccatttcattttgagttgattt
ttatgtatagtttaagttaaaggtccaattccattctttgcaatgtgtatatccagttttcccagcac
cattggttgaagaggatatcctttcccagttgtgtattcttggcacccctattgaaggtgatgctagg
tttatttctgggatctctattctgttccattggtctatatgtctgcctttatgacactatcgtgcgct
cttgactgaggtagctttggtaattcattttgaaactagcaagtgtgatgcctccagtttattcttct
tcctcaagactgttttggctatttggagtcgtttgtggtttcatatgaattttaggaaatttacctta
tttctgtaaaaaatgcgattgggattatgataggaattacactgtatctgtagatggtttggatatat
agacttttaaatgacacatcagatgtatttccatttatttttgtcatcttcaatttctttcaacaata
tttcatagctttcagcacacacatcttttaccttcttggttgggtatttactaagttatttattcttt
ttattgctattgtaaatgagattgttttctaaatttcctgtttttatgttgctagcgtatagaaacgc
aactgttgaatgatgactttgtatcctgcaactttgctgaatttgtttattggttctaaccatgtctc
tgtgtggcgtcactcttaagattttctacgtatcagatcatctaatttgcaaacagatataattttac
atcttcctttccaaatttgatgtattttatttctctttcttatctaattgttctggctagtacttctg
gtacgattttgaaaagaagtggcaaaagtgtgcattcttgtcttgtttctgatcttaagggaaaagat
tttcagtcttttgccattaaatgtgatattcactgtgggtttttcatatacggtttttattatgttgc
ggtaatttcgttctattcctagtttgttgtgtgtttttatcatgaaagtgttgaaacttgttaagcgc
tttttctgcagctattgagatgaccatagatttttagcctttgttctgttaatgttgtgtatcacact
gattagttttcataaattgaaccatttttgcattccaagaataaatcctatatggctctcgtgtataa
tcctttcaatatactgttgagttcagtttgctagtattttaatgagttattttgcttctatatttatc
agcggtattgttctgtacttttctcctagtgtcttttattgactttgatatcaggatactgatgcccc
ttgtagaatgagcttggaagtgttctcttctctttaatttttctgaagaatttgagaaggattggtgt
taattcttctttaactgttcattagatttcaccagtgatgacatttggtcctgggcttttctttgttg
gaaggttttggactactgattcaatctccttactagtttcggcctactcagattttctatttcttcaa
gattcaatattggtagattgcatgtttcaaggaatttgttcatttttttctaggttaacatacagttg
tttacagcagtgtcttataatcatttgcattctttttggataccagttgtaatgtctcctctttcatt
tctgattttacttatttgaattttcctttttttttttttttttttacttaatctacctaaagatttgt
caattttattgatttgtttttaaaaaaactcttagctttgttgatttttctattgttttctatttcaa
ttttggcttttttctgatctaatcttaatatttccttccctctgctaactttgggcttagtttgtcct
tctttttctaagtctttgaggaagaaaatggcaaggacatgactttctttagcagttggaaggacaat
gctgtaaatactcaaaaattaattatttttatagtgacaaaaacaaaataaaaaacacttcaaagcaa
atgaaagtttatcatttaatttatcaaatcactaagcagactgcttgatcagagagaagatactcata
tgatcacataaaactgaaagattaagaggtaaggacattcatgttatcattacatctaactttcttat
ttccaagatggagaaactgagggttggagaaaaagaaagatttctttgttagatacaaacagacagga
ctaaactcagtatagcagcctcctaaattccaaagtatcatgatactgtgattttatgcattcttcag
aaaaatagtagagccactggattctggcaaagaagttatataaaatgtcaagttcttcctttgcctca
gaaatgaagttttatcttccaaaattgattgggaagttctccttatacctcacatcacgtctactatt
ttacattgtttacttttgaagaatttttttaattgacaaataataattgtacatattcatggagaacc
tagtgatgtttttatatatgtaatgtatagtgatcagatcagggtaattagcatatccattatctcaa
acattggtcatttatttgtgttgggaacattcaacgttctccttctagccatttgaaacttctatatt
attgctaactatagtcaccattcagtcgtatagagcactagaacttatttctcctatctagctataat
ttatttttaastatgctttttgaatctgttactataaattgaatgtcacatcgttttgaaaatattct
taatttatgctcaacaggcaagattacacacctgtgataatatctttaatttaaaacattactctgtt
taatttaccagaatatggaaccctagtcattttagaggtggagcaaatttcagtgataatctagtgca
aatttctcatcttatgaatgaggagattgagtctgatataagggacgagattttcgtcaatgagcagc
ttgttaacattagctctgtgatagaacacaggcacttgtcctcccaggccggtgtttcttctactcta
tgatgggctgttttgttgtagtttttaaacagcagcattttcaccatgcatagttttcttccaaagtt
cgttcttaacgtttttgcacagaataactagattttggaagtagaaaaaggaaattctctttgcatcc
ttgtatctctggttattttctttgtcctttgatctctctctcctcccctcccctcccctcccctcccc
ttcccttccctcccctctccttcccttcccttcccttccctcccctctctcacacattagagaaagag
ttaaggtattaaagaatacataatactattaaatttccttcacatagagaaaggaatgaaaaaaagtg
aaaaatggtcctcaccaaatgtccaaacttctgtaggtcatttccatagtatcagcaatgtcctgtat
ggtgcctcggggatatgtaagcaaatgagcaagtggttagctaattctagctttggcaaacacttgtt
atggcttacttgaggagaagtcacttctccaaagtgaaaataatgtgcacaggtcaattagaattttt
ttgtagaaaaggaaaatactttgtagggacatggatgaatctggaaaccatcgttctcagcaaactat
tgcaaggacaaaaaaccaaacaccgcatgttctcactcataggtgggaattgaacaatgagaacacat
ggacacaggaaggggaacatcacacaccggggcctgttgtggggtggggggagggtggagggatagca
ttaggagatatacttaatgctaaatgaccagttaatgggtgcaggacaccaacatggcacatgtatac
atatgtaacaaacctgcacgttgtgcacatgtaccctaaaacttaaagtataataaaaaaaaaaaaga
agaaaatacctccttatgctcctgacttattttctttttggttcctcagtcctcttctctctctctct
ctctctctctctctctctctctctctctctctctcacacacacacacacacataccccacatatacaa
tatgattaaggatatatgtgaataatgaaagcttcttgtgtatagatttagaagtctaatggacaaaa
tcaatattttcctatgtgcatttaattcccccctttgatttaggtatatagtctttttttaaaaaaga
gaaaaaaaattaggtgaccttaaggtatagatcctactttcaaaaggtttacagaactagggagagga
acatggacaagatttaaagaactattttaagcagaataaaatgtgatttatgaacaaagcatatatta
tttgtgcgtatgtgtgtgtgccaacaaagatgcaattaggagattgcacagggagatgtcattagaac
caaccttaacgggtgagaagtctttgaagacatttagaacatggaagatctctgacagagggaacaaa
ggcatagtgacaaaagtcaagggcatatttaggactggagagtggtatgtgtggcttgagagtgggcg
agaaaaaacaacaatgcctctgtaataggaaagtagacagaggcatgacattaagagctttgccagct
gtgctaaaagtagtgaacaagagctaacaaagtgaagaaatgtaccttttctgatgtgtatcattccc
ttattcatatacttcttgagggggaaattcattctgtgttgatctagtaaactactacaggaccaaat
gataaaaagaagtataggaaagaatgtttcagcatactttacgagataacttccttgtagctattctc
catagtattttgagcatcacaaagcaatgagctgaaactgtctaagccaaaattgacttgtcatctgt
tagggatgcttagatgagaattctacatttgagagcttcttagattcattgaccactatgtcccattc
taagatccatgaatgcgtgacctaactattacaccttcttttagtctgattgtcaattttgtattttc
aattgtgcaagtttctaaaactattttaggaagataaatctagcagtggtgtgggaatagacaagaga
gaaggggaaagactcttcaggaaactaaactcacaatttatgagtattctttattgcccaagtcttcc
caaagtctttcatcaagaaagaggcattgcaactctccttttatagtttgtttttattctggagcagt
gatgttttggtggagttgttcctcagtgcgtaattaaagggcctatgacaattacagttcatctcctg
ctgctcaaggtactgcagatatttggatctactactctcattcatttccaattaatgtcagctttaga
tttccttcagtatgctatgttataaaatttgattatcgttgtgcccaccttcccacttaatttcaagc
aggtttctcgattacctgactaaactaatgaaatctgactaacccaatatctgtggacagtagtgtga
tgttactgatttttgtatgattagtcaagtcatattcatgccacgttttcatatagtaccataaagga
tattcttctcgtggtccttttcttttattctgaacatacaatgagaagaccggtaaagtgggctagga
aattaaagaaaaatacaaatggcaaaaaatatgggtcactcgaagtctagaatagagagcacaatcaa
ttttgaattaaggggtgataaggtgatttggtcaggtgactggtgaaacaggaaagaaactatacttt
ttgaagtgtttcatccatgtgttaagattcatttggggtcaagaatctaaatttcatatccctgggag
tggaaactaagtaaaaaaaaaaattatggaccttggtttaatagctagaggagcaagagtgtatcttt
atgtgacttaacttctatgtgaaaagtgaaccttaagattaattattgggggaatttacttactcagg
ttctatgcctagatggtctgcccaactaagaaaacttattttcctgttactccatcctatttttcata
cttttatactgcacttgcagaaaagcatatatttctacccaatacgaaaattcctgggaacatatttt
tctacatttcccaaattacttcaaaaagtaaacttaggttatttcatgatctccattacaatggacag
gtggccttattgaatgttgtcctgtgaatacaaagatccagagtttaaagaacaaggtgtacttgcat
ctcccacttagggtttgcttgtggtggagagagaatctagtttgcttaaaaggatgacagtgcagtgc
cccaaaatatctgatatcattaaaagtctcatatttgtctttcgtaacttctctagggctgtcgatga
caggagacccttaactcctatgccttgattatgtgaataagcacatgaaaatattttagttatcttag
ttcacttttaaactaagtttcaattatcactagattctaaatatcatcattgagccgttcttaaggaa
ctgattttctacatattcattcacttcacctatatctagtgtgtctactatttgccaagaaaaattta
ctctcttaattcagcattccatatacttaacatcataaaaagtaggccatttttagttttctaaatta
tttatttaaacatttctttaaaattacattctatcattacactatatttcaacactacagtaagcagc
ctattttgtgatttttccttatataaaatacataattgaaattaaaaatgaagttaccaagagccatt
ttcactctggggaatgcacatttataaattatggggttattttttcttcatcagctttcatattatta
aactttgtctcttcataattacagagatgactagacacagaagggaatttaacatttggtgtgcattt
gtctaacctatactttatgttagaaaatacatttccatttgaaaaaaaatcagtaattgtgggtgtga
tcaagagggcagcctgaaagtcgggtgatgtgactcacacctgtaatcccagcatttttggaggccaa
ggtgggattatcgattgagcccaggagttcaaaaccagcctgggcaacacagtgagagcctgtctcta
ttagggggaaaaaaaaaaaaagaggaagttagcctgaggcaatgtaaatgaaatacatatttcaagga
tatttatacatgattcacgttattcatataaagatgtgccagagaagactataggtacgttattttac
actattttgctaggattttaagaaattcaatgtgtttttatttcagttaacttagaaaacttacctaa
cttatacttctcatggacacaaaagtttttaaagataggatcaaaaagcccacatggtgaagcatttt
gaactggatgaaaaacatctattatctttaaaattttatgatattactgattgtaatagactcccttt
ttaagaaatcattccttatagaacataaggtttacatttacaatcaacaatttctatccttactacaa
taaaggcacatataaaaagtacagttgcatatttagcaggtttaattgtacattttaatgtagaaatc
aattcaattctttcatttatcagcattattacagtgatttcaaattaagcataggtaactttgatata
gataaatgatgtacacagcagttaaattttattttcaattatgtagtaattgtataacctaggcagta
taatttgtaaactttgtattttattattatgcttctcccacttggcataagcacaacacttcctaaaa
gcataattttctatagacttaataactccctaaaaacctgttttggacccctatactatttgatatag
gcagaaaaaaaacataatccatgctcaaatttgaaaaatgactggtcacatttggtataatactaaag
gtaaataaaatcaagagtctatgaacatttccggacctgcacatttgttttattaaaatgcataattg
tctttagtgtgtttctatttgtttatactctactgattttaattaaaaataccaaaatacgtttatta
aaaaactgtcagaatctaagttgttaaatatacttaactaggaaagtaactgtttaaacgagataatt
tatagagaaatgtggtgtattgccaattagatgtcaagatacaatacaactgataatgaaaaagtagc
attttcttagggatggaatacagtgtaaggaacaccccagtaagaatacaaaaattactgaaaaaaaa
tcttccttcctgaaaaaccaagtgcccttcaagtgcagaacctcatccaactaattgttaggtatcac
taaagcctgataccttcaattttctggatcattcaagctgtatttttgagtccttatactagaggagg
taaagagctataaaaacacttaatggtatctgatgtgaactgtggatcactttgacccatcacttcta
cgtctacatcttggataaattcccattgttgtcatagattgtacaggtttaatggtgcgtttgtggag
ggggctcgcttatagaaaatggagactctgaagggataaggaataaatgtatcacttcaggtctttta
tttgaaattggggtccagagagcctttttgtatcagacttgtcaaaccatttccatttagtaattata
tatgcactagcacttattcctacttacctcacctctttatgcccatttccttgtagttgcggttatgc
atgaataatttattgcaccccttaccaacaatggaataaaacttccattctgaaagctttccatactc
atttccaatagcaatagggtttttttaacggacgtattacaaatgtacgagtcagttgaacatagtat
tcctctttgtaagaactccaagtggatgcatgctgttgtctcaaatctcaattagaccttgctttgag
gtcccttcattgccagtcatctgttctccttcccctgacttgagtatttctccagatatagataatac
attttcccaactctgtgttccaagaactgacagtggctttcattcattttgtttgtttgtttgtttct
tctcgttctcaagtatcccacagtctactatttcttccctccattcgtttgtcctttcagagtttcaa
aatccagcataggtacttcttctaaaatgtcttacccttcacatacacacaccacttgagaccccatc
agcctctgtccacacagtttggttacattcatagactatttttatacatcaaaatatttgaaaatttt
agggtaaatctcagtagtcattcatttttgctcttattcaaccaatactagtcaatcagcctgtgcca
ggttttgttgcaggtaccaggtatccatccataaagaaaacaacgtccctttgttgtggaatttacat
tttagcaggggaggcaaagaacccaataaatatgataaaatatcagattaaaagtacgatgaaaaaaa
tcatcagggtaaaggaaaaagggaagcagtattttagcaagagtggtgaagagaggaggctgagagtg
tgacatctgagcagagacctaaatcaagtcaaggaatgaaacatgctactatctaaagaaatgagtca
ggataaggaactagtaagagccgaggcccagagatgtgaatatgctgttccaggaacagcaaagagac
tggttgatatgatgtgaaaaatgagaagaaaccttatgatatgtgtcaagagaaaaaaaaaatttaaa
agcatgcttgggaacggaggcctccagatgaaaaaaaaaaacacagttcaaatccttgttcatgcatt
tagtttgctttgcaatcttgggcaaaatgttaaatttctgtacgttttatcttcctcatttttaaaat
aggcacaaggacatctacttaataggttcattgtgaggagtaaatgagatgatatatctaggatgcct
ggcattatatcatacacttaataatacactgaataaataatagttatgtctatttatttccttatcgt
ttttattattatttcaatgcacagacctgttcataagataatgataaatattagtggcagaaactgaa
gatgttataaattattaggaggcgggaccactcagttcaatgtatctgttttaatatagtcagcaaaa
gtgtgaagataccaacaattaaatttcaatgcattcttccatttcactagttttataaactgatgaac
taccagaatgtcaatgtatgaattgcatactcattcttaacaaacagatttgcaaaattatgtgtaaa
attagccctcagccttccaatttgttattgtcatatttcatggaaatacataatctgtaaatttttgt
tttaatgatatgtgaaactgcctaaagtagagtcttggcaactacttcacatttgtcctccagagata
gtggataaaagtgtcaataaatgaacactctatattcactaatcacaggcaagggacaaggaacagag
tggtcacaaaataccacaaaattaaagcacattccaaattaaatatatatgtttttattacagataat
gtttgctagactctttctaattatctgcaaagattttaggaatgttttaatgttttaatatttacaca
cctgtgtatttcaagttcagtcaaacactattgttaaaactaaatcttctcatctctaataataagat
gtgaacttatcttggaaggtggttattaggatgggagagataatgtatttcattcaaagtaaaaatat
ttctctgtttctatctttctctttctctgtcatctatttatcatctatatccaggtatctatgcacct
atgtagactagcattcaatgaaccatagatattattagtagtagaattgttactaatattaaaataag
aagtatttaagaagaaacatgtcctaaagcataaggtcaattattactctcatgttttttggcatatg
aagcctaaaaagtgtcaatttcaagagagtattaataaagattgtgataactgaaaggttcctgcttg
aaattttgtgtggtcttacaaatatataaactctaagcatttcagtgagccaattactgactaggcac
tatgtcttatgactcttttgtcatagtatgtaaaaaacaaagagtagagacatcataaaaattatagt
agatgggcactagggaattacgcaaaataatttgtagatttaatgtgaaaccaaaacatctgttcaag
tcaatttcccacaggtcatgtggcaaagagtatgagttccagactgaggagaggaaaaggttgttctt
ccacagggaaataaactgagtgtaataaacataatttttcttcttaagcattatttaaaacaaaaaaa
atgccattaaatctatctttcctgcctctcttatcaatgctcccttccctttcaccacttgtttcaaa
ctccaagccttgggattttattttggctttttgccttaatgtaactaaaatgagagcatcacaaatat
gaagctcatcaaataatttagcagcattttcccctgtttttaactttctctttggaaacgtagatttc
gaaatttaagggcccaaaatatgaaatgcaattataataggccatttgttcattcagcttgataaact
tgaataaatagtattgaacttttaatgcaaaaagaacaaaacaaaatagaactctccacgaagaaact
tttcaatgtttgcatttctgtgtgaggagaagggtaatgaatgtgggaaccttaatggaatccatgtt
cttccagtgatgacaagggtcaaaatggagaaaaatggtcactttctacccagtacattatattagtt
ctatgtggacaactataacatagctgatgctggttttcaggccataaatgtaggtatgtattttccta
ctatttataaggcaaaatttctatttgtttaatgatttctatataggtagattattctgtctttagga
ttaaaaacgacctgtagaccaagagactttctaatgtccaccttagagtatatggcttttactgttac
agtttccatttcctttgcttgcccctttgagagaaggaaaggagacatttgggatacatacatcaatg
aggagctattaatgaataaatgaatgaaattgtcagtcaatttatccacatgatcatcaattgccaat
aattttatcacctctgtgggattaagtagaggtaacagtttagaaatttgattttttgaaagcattta
aaatgttcaaatatatcactctggtaactaagggaaagtgtattattttcttatgcttagtcttattt
tggttttgcctttttaatttaaattgaacacttatatcaaagagcttgcaggattataatttgaattt
ttgaagcaaagatcattttcttaacatcaaacaaagagtagatacaataggaataaaatcggcagaaa
aacaagagtatcaaggacagacggggagggtgggtctgtgttagcatgtattgctatgaagaaatagc
cgagactgggtaatgtatttttaaaaagagctttaatcgattcatgattctgcaggttgtacaggaag
caggacaccagcatctactcagcttctggggaggcctccgggagcttttactcatagtggaagatgaa
acaggagtaagcatgtcacatggccagagcagaagccagggggaggttgccacacatttaaaaaaaaa
aaaaacaaaacagatcgctcaagaactcagctgctatcatgaggacagcatcaagctgtgagggatcc
acctccgtgactcaaacatctcacaccaggccccaagtccaacacttggcattatatttcaacaagaa
aaaaagtttaattggctgatggttctgcaggctgtacaggaagtgtggcacaggcatttgcttggctc
ctggggaggcctcagggagtttttgctcatggcagaaggtgatgcccacacactttaaaaaaaaacca
gatctcatgaaaactcactcactacactgaggacaatacaaaaccatgagggatctgtccccatgacc
caaaaacctcccgccaggccccaccaccaacattgggaattatatttccacttgagatttgagtggcg
gcaaatatccaaactatatcagggctcatgtccagttatatgtcaacatgcctgcattcgaaacatcc
tgtccaaatcactgccttgtcataatacttatatttttctttattgaatacgaacacaagaagattaa
ataatagcatttctactttaaaacagtgggcaccatattaacattggaataatagtagtaataacgat
agtaataacaatgatataggctgggtgcggaggctcacgcctgtaatcccagcactttgggaggccaa
ggcgggcggatcatgatgtcaggagatcgagaccatcctggctaacacagtgaaaccccgtctctact
aaaaatacaaaaaaattagctgggcatggtggcaggcacctgtagtctcagctacttgggaggctgag
gcaggagaatggcgtgaacctgggatgcagagcttgcagtgagccgagatcgtgccactgcactccaa
cctgggcgacagagcgagacttcatctcaaaaaaaaaattaaataaataaataaataaataataacga
taaaaggatatgtgtaggttttttttttaataggctgttaacattaataggcattgtgatttcaggga
tatcatcaaacatcctggtcctaagacatcccctattgaataggaagggcttaagttaaacttctcat
gagccacaattttctgattatatgtttggtgtgtgtaatagccacctcagtgatgatttgattagcct
ggacccttacataatcattgaagtatacccatgttcctttatatacttctttagtgttgaaagctcaa
aattaagcaaaatagtccccttgataatgtttagattcttaacatttgctttctaaagctggcaaata
ctctcttcccagtgtcatgaagttaaataacatgttgcttagtgaggactttaatgttgccatgccat
aggaagaccttattcgaaatccccttacctgggagaatgtcagattattaccccccaacttgtttaac
acttttaggattttaaaggtgttcacatttgtattagaacaaaatactattgagaaacatttctagaa
aaaaattatctttccaaattaaaatcagtggtatgtaatgtaggagtctgattataatgattaaaata
catgggctttgggcatactgcctaggtgaaactcctggtttattgcatcactattagtataacctatg
ggagttaacctacgtaagcctcagttaatttttctctcaaattgatctaataatcgtctctcataggc
ttgttttgatagatatttcagtgtatataatatacttaggacagtgcctgatatcagtaagtctcctt
ataaaatgcctttgtatttattcatgtcaaaggaaatatgcaagtattgcattcacttcctaggtgcc
tttttgaattgagctttgcatggttagtttgtataaaaggttcagtgaactttctcataatgattttt
tattgaacatatggaatccattaagtgttagcaaaagtcactatccactgagctgtgtccaggggctg
acagttatgtctatctcttgcaaaaataaacacatacataaatgcactaagacgtatattacctgtcg
tcatctcttagagcatttccatttttcttttaagttttttctttcaatgggttttttatctttgtgag
tacatggtaggtgtatatgtcaacggggtacatgaggaaggtgtatatattgatggggtacaagagag
gttttaacacaagcattcaatatgaaatagtcacatcatggagaatgggttatctatcccttcaagca
tttgtgctttgtattacaaacattctaattatactctgttagttattttaaaatgtaccattaagtta
ttactgactatagcaaccctattgtgctatgaaacagtagatcttattcttatttttctaacatctta
gaacatttccacaaacactacctgcttgttaaatatacctattctaatcttcatataatcaattactt
ttttcctctagaatgtactatgacacatccatggggaaaatgtagtaatctaattaagactatttcct
ctcattttatatttaaaagaatgtgctctatcaatttatttacttgtacagccgtaggcaacctctaa
aatatttaaagttcttaaaagtcagatatttcagttaatattgtgattatatagttgattttgatgaa
catgttcatctaccagaaataaattatacacacacattgatatggttaggctttctgtccccactcaa
atctcattttgaattataatccccgtgtgtcaagggagagaccaggtggaggcaattggatcttgagg
gtggttttgcccatgctgttctcctgatagtgaatcatgagatcagatggttttataaagggctcttc
ccccttccctcctcactcattctccttcttgccaccttgtaaaggaggtgccttgctttctactatgc
cctttctactatgcccttcaccttctactatgattgtaagtttcctgaggtctccccagccatgctga
actatgagtcaattaaatccctttcctttataaattacccagtctcaggcagttctttattgcacata
tatgtgtgtgtatgtgtatgtgtgtgtgtgtgtatatgtatgtatatatgtatacatatgtgtgtata
tgtatgtatatatgtatgtatatatgtatacatatgtgtgtatatgtatgtatatatgtatacatatg
tgtgtgtgtatatatgtgtacatatatatatatatatatatatatatatatatatatatatgaacaga
gagagagagagagagggaggaagggagagagggagggaagcatggagaaagagagagtaatagcctaa
atagaaataaaactagctccaagtacaggttcgtcaacactctcctatcatacccccaccaaagttaa
tgttaaccacttggagccctgttcttccttagttgtggagtactttagcaaaattttaaatctaatta
tgcctaattcaacgacagtgctaatttgaaagtgttagaaactgaagacctataataataatgagagt
tacaaaacataaatagtgagacaatgatgaatgtagtggatgcatgtacgagggctatcatttgacag
tagagatgatgctcaaggacagacaatgagtctttcaatgtgtggagaatgtgctgctgttacagtga
tgtacaggaaagaaacaaaaactgaggaagtatcagtaaacaaaacactcaaacatatgagtatacag
ctagaataaaagcaacagtactagatgacaataagcccaatgttaactcagaaagcagaaggttttta
agaatttggggaatactgtggctgatgatacttatgtctcaagccacagatgccatatgggctctgcg
cccagttgaatcggcaccacctggcagtaagtgggcaggtccacgactgccaggacatcccttccaac
acttgtggagatcaccaggaaggggggagagacctgccttgacagattttcaatgtgggcgaaacagg
tctattttgagaaaagatgttcaatagaacatatgtcagcaaggaagaagagatgatgcttagttcta
aagctccaaagagctggcttacactccaacttggggaaaatgcatccgggaaatgcaagattaatctc
atcttagccattcttttgaatggatggacatgacccctttctacttgaagacagaaaacataaccata
ttgatttcaggttttcttcattggtttccatttaggattgttcctccccatcttctttctgtgtaggc
atcccagttcccaagtgttcatgaagcacgtatggccttcaggggatgtgtctgtatacattgttatc
ttatggatgcacggttttgtctgcaccttggttctgaatgtctttactcttgagcatctgcccatggg
tccccttctcaaggcctcaatttcttgagtttaacactgcatggcccatgcagcttttcagttaagca
tctcttgctatgaccaactcttttcctcagtcaactcccacactcttttcagggacaggaaaaatgta
gccacttgctggctgcactctgaggcctcaagaaatttagtgaatctgcctttgcccttcttgctgat
gaaatactgccacatcaggccccctcttcggaaacctacaagcatctaattttcttgcttcctcccca
actttctttttgactcccccccatccagagagttcttatgtctactgtactaggaaaaactcattctt
aaggtatggttttcaaatcattctctggtctggactttagctacggttttaaatgaagaaacaaccca
gagccaaaatataatgaaactatttccttcttccacagagtggaaactgctttggggttaaagggcca
gtgaaccaaatagaaaaggatctcagggaacacagattgaagagagagaagaaaaaatatgaaggcat
tgttggttctcttttgagtttaaaatctagtggggattgtaagcacacacacatatacacacacacgc
ttacacacacacaccagtgaagttatgaaggattttgtcactccaacgaccttgaatttgattatcta
ggtcagttgttaccaaagtggaatgtacatgcccaataatatgcgtgctaaacagttggggtagtgag
aaaaaatactttttatttatcttgttctctagaaattaatattttgattgtatattttatagtgtatg
tgatgtgtaagttgtgtctacaaaactagtgtcaatgtaatttaaaattacatatgtctgtgaatata
tatttatatagggtacatgcttaaaatgtgtttacttctgaggtacatgaacatttttcccccaggca
cagaaagacaaataccacatgatgtcacttaaatgtgcaatgtaagaaaagttgaattcatagagatg
tagagtagaatcatggttaacagaggcttgggaggtggagtgagggaatagagagttactgttcaaag
attacaaagtttcaactagacagagggaatacattttgagatctatttcaggaacattttgagaccct
cactctaagtaataggaaatcattactttagttaacatatttgaatatgagttgtgatgttctatatc
gtttatttggattctactaacccacacctagatttttatggcattacctttttactcactgtgaatat
cctactcatagacagatgccctgggaacttggacttgaggcacccaagaactgagacagtgagatttg
ggggcacaaggatctatggataagttcatcttagtgatgataaaatcaatttggcatgtttcacggac
agtgtgcattttagaaagggtaaagacttggaaacgggatatttttgagcccaagtgtttccaataaa
tagctgtataatttgaagcaaataattgattttttgttctctttgtgccctcgcctgtaaaatgggag
aaatgtattcctttctcatccttctcatgaggccattgagagtatctaatgagatcagactgtgacat
agcataataattctcatttcttgaaggcctattatacactttgcaagcactgtatgtgttgtttctac
ttctcttgttcgtttttcctggaataaatatccccccctcctttacattggattgccattattcaccc
tgtaaggaaggcttcatggttctcattttcatctgagaaaacttaggctcagagaagatcagtaactt
atctaaaacacacacatacacacacagacatatctatgcccattattcttaacctagtttctctattc
aggagttatctctgctgtctctgcttctgattataatctgtgtaagctgatccaagtgacacgattac
agggaaattgtaagccctttgagagcagagactacctattgatatctacattttaaaatttgatttta
gccaacctgtttatatgcaatgactaacaggttagtttgacttgcaataaatattccaaatcctagac
taagtaaatttattaatgtaatgatttaacttgattttttcattggcatgtttccctgaagtcgtcat
gcaaaattgaaaaaaaaaaaagtatagtgtgtgattctagattgaaattcaggaatcctccagggtta
ccttgtttgctttccaaatagttcagattgcttagtctgaccaacaaggtccctgacacttggaactc
tgtctatccctctaattgactttgtccctgatgacctcgcccagagatactcttcaccccagctatac
tgtgttgctagagtttctctgatatcccatgctattgtttcctttgttctcttcataaggtaccattt
cccacccgccaactcctgttttcctgatggacttttgtttcaccttacaagatcattgctaatgtatt
tattttgagaataaaaagtgtaggaaaggtcacgggacaaagctgtacaccagacctttcccagacga
acctagtgtataatctccctagtccaacatcatggcttaaggcagtcgatagatccgtcttaatgtcc
cttttgagttttctactattattatatgaggatttatttttgtctgaattcctccctagatttgccct
agagagcaatgactatttacagtttattcctctttgtatctcttatgttaaggccagaccttggcaca
tattctagctgattagaagacgtttgttgaatgaccaagtgattgaacaaatgaccatgtgctctgcc
acagtccggtcagttctactttggtttggttatgtgtttgccacattaaagttgtagcctgggaagtt
cagttgtgagatgtctgcagaacatgaaaaattggaataatgaggttatttctaaaattgctataatt
taaaataaatagtggtttattccatatatgaatatacactggaaacaaagaatttctagaatactgga
gattcaatgataacatcattgaaattaaataaataataggattatgctagttactttctaatttacta
gaaattgaccgtgtgcatggcacgtataatgagtatcatgggatagttacaaaaagtggtgcttagtg
agtttctgtggaaaatctcggtaccaataaaacggaggatttccagaaatcgatattcctcaaagctt
gacagtatttatgcacggttacactttgtgtgtctttcgtttgaatcaatggaaggaggctataactg
aaaattattgttttagtgtattatatctttaataataagagttttaagaatctatcattagaaataat
tattcctcaatttgtaattctcaacatttgaacaaataaatgctctgtgtctatcagttaatcttgcc
catgaagatttaataaagcacgctagtttttacaaatgtgattttagagatggtcattacttggtaaa
atattttgtgttaacacttccatgaatatgttctgtgggaatatactgcctccacattgcttgctcat
gaagacatgatttttcacatcatcctatcagtattttgagaaagagattgatcccatattctatgagc
atttgaacattctctagtatttttgtttaatcattaaaacaacccttgaagtctatgtgctacactgg
ttatttccctcttgactttcctttacagataaccctctatcataaacaacctatctatatttgttgtc
tccacatcatgttcccagccctgctttaacacactgcacattgacttctagcagcaaaggctcatggg
aggtactctcatcaaggacactgatggtcctcatgttgctaaatttggtgggtcctctacagtcttta
tcctagttcaccttattatggaccactgtcaactctgttctgcttaaaacactctgttccttgcttat
atgactctacactcttaactcctttgtgaattcctcatctgcccttccattaagtattgacgacatcc
ttcatagttttgatctaggacctcttttcctcttacttgacattatgtgggtaatcttgtctttgaac
gcaattaccattcttatgttgatgacccttaagctataattccagcccaaatcatttttctgaggaag
ctacaagaatacacaaatgtctaatagatctctatttagatgtccctcaggtgcttcaagcttaaaat
actcacctgagctcatcacctcatctataaattctgcttctcctccctggctccctgatttatttaat
atgaccaccatccacttagttgaataaagcagaagcctggacaccatctatacctccaattaatcact
aagttttgttgttaaatacgttcttacattttctctctagaatgtcttattttccccatctttacacc
caaaaccaaaagtcagatgaccctgatctcctgcttagatttcaaaacactatctcttgcctagactc
tggaatttcagtcttgctcctctccaatctatttctacaccctagactctggaatttcagtcttgctc
ctctccaatctatttctacacaaaagctagagtaattttttaaaaaacaaaaatctgaatgtgttcat
tttctgcctaaaagccttcagtaattcttatttgttcttccagggatagagtaacaactttcagacct
agtttattagctagttctttaaccacaaaggactctctcacttgtctactccccctaacacacttcgc
cctaacctttgccattcctccctttcccttttccttcccagatggacttaagtcctttcagattctta
aatgtttcttcctccagtctcttacatctcttttccttgtaactctaaaaactacttagcttacgcaa
ggaaaaaggtctgtacaattcccggaatcagcgatcctaacgttccctgttgtttttttcgttgggac
atgaattcattcacagtggctctaaacatcaccacccctgcctatctctcccattcctactttatctg
agcttatccatactcttgaagacttacatattttttttctaccaggaaatcattactagccttattat
cccactgtccaaaccaataagtctgattaggtatctgtatatatttaatattactatatgtgtttttc
taacactctagtagaggagaaggtgtatttctttctgttttttagaagcctgtatttctgctattata
gctcttaaggaactctcatgcaattgcctactagaatgtaagttacggtaggataagaactggatcag
tcatatcacacatccacatataggacctagcaccatatctaacacacagcaggtactcaatacatttc
tttcccaaataactaaagagtttaaacaaaccaaaatgattaaatgagaagtaactgttttggtaatt
cttgtgtccttactagagtctaaattgagtgatttttatatcatcagtttatactcccctttcccaac
cccaattctttcttttttaaattttttaaatcaaatatgccttaaaacttcaggatcagttgagtaaa
atgatgcttttgtcgtcttttgcaaaataattgtatttcagaattttgatttagatattataaacaca
cctaaaataatagctttagtcttaagatgaagtgcttcttaaactccctaagatgggttggactatgg
atatgaacatggacaatatcacattaatttgtgtacacagttctaacacagggtctggcatataagaa
caagtcagtaaatagttgttgaatggaattgaaaatttaagtagcaaataaagtattttgacctacaa
agcaagaaatcacattttcttttttgtcacagttccttaggaagataattaattttttagtatttaag
gatgttaaatatttattttatgttctatttactaggcttctttttatgaaaattaattggtgaaaata
gcgtacatatcttcctttaccagaacatttacattttgggcagtaacgctggcttttgttaaaaaagc
aaaatatgtgtgaaatttatgtttgagttgatttcaatgcattacatttccattttaaatcttctttg
aaatactctatttttgacaccatgaaactgtattagatcttagtatgttagcaatgttttgcagtttt
agagccataattattttaatgaccactttcagcatatacgttttctacaggaaaaataatctcaagaa
catgaaaagtgaaatctatattttgggtttcaaaatgatacattttagctaaaatatcatagttttaa
tttctcagtgaaaaatatagtgtggtaatttatgaagagactcagtgtttaaaaattatgactctata
gtcaagtttatgtttataggacataggttattcaattacatttaaaataattaatttagaaaatgtga
tcaatgtaacaaattttacctgttcttttctaaagctaaatttgttgtttgaagtgtttcttctaaaa
tgctaatgaactatcaatttaattgttgagcttagagttagaaacttaattatattgccagaaataaa
gaaacaaatggatcccaaaagattcacacattagaaatgtatgccagggaaatgcttttgaatgtgtt
caagtcatggcttctaactcgtaacttataacttgtgttatgtctggcttcattcccttaagaaaaag
gaataataatgccttcggagagcatcccagctgtaagagctatgcattggtgtctaaaaaagcttctc
actcctcataccatcctggtctgggaatttaaaaaattgtcatcttttgataatctgtatcacatagt
cttctgcatagtcatatgaggttagaactgccccataacttttgcagggcctatagtaagtgtgcaaa
tggttgcctgcatgccacatatttaatatttataaggtataaagtcaacagactattaaatatatcct
atctgctttccttgacaattatacaatcataatgatatggacatctagattcgatttagaattctctc
tctctcattttctttttcttctttctttctttctctttctttctttccttcctttctttctttctttc
ttctttctttctttctttctttctttctttctttctttctttctttctttctgtctgtctgtctgtct
tgtttttttaaatagtgcaagcagtttattccctggcaaggaatttggaaaaaactcaaatagcaaac
cacttgatacaataaaataaattccttagagttttgtactggaatgaggcagcttggttagagctaac
cctaagcctgttatttaggatacattggcttttcttaagcttaaaaaaaattttactgtgttaatgac
atttaacatgagatctatcatcttaataaatactacatgcacaatacattattattgactctaggtag
aatgttggacagcagatctctagagctaattcatcctacttaactgaaatgtaatgtctgttgattag
taacttcctatttcgccctatccccagcccctggcaaccaccagtccagtctttgattttatgagttt
gactgttttagataccttatttcaagtaagtggaatcatgcagtatttgtctgtgtctgtcttgtttc
acttagcgtaatcttaaggtccatccatattgtttcatattgcagaatttccttttataaaggctgaa
tagtattccattgtgtatatataccacatttatctattcatctgccaatgggcatttaggttgtttct
gcatcttagctattgtgaatctgttgccttttttccctacctcctttactccatcctgcactgtgagg
aactctgtgcacatagatctggtcgccccatttcccacccacatgttcaagtttttcccactcacctc
atgcaaagatttaccccttagccatacccagtaactgactttgaaacatttgcccagggagttgaggg
attctgaatgccagatcatgggagcggggcttctagtgagcatgttggcttggtcctacagactccta
atcagagctttgcctttgaaagcatggggcccaagggcaaggaccctacttgttaaggtctaaatttt
ttttctgaaataaccacatcgagcttttatgtgtagatggcctaaattgggctaacccagaggcagtg
acactcaagtagtttacatctaagcgctttccatgtgcttcttttcccatttctgttacttcttacaa
aataaaaaatcagcatctcaattaccctgatttgatcattgagcaatctaaaaagtatcaaaatatca
catgtagcccccatatacatacaactgttatatatcactataaataaatatatacacattatatttaa
aaatcaatactttaattttacatgtttaacaaatcactagcatatacattccagattgaacttacgag
ggatgtggaaaagattcagtgactaaataacaataaagtactctaaaaatgaaaatgtgaaatggaga
cagtataaatctaaaatcatatcacttatgaagtattgtttcaaataaacaataaaatatatcttcaa
tcaatttaattttattttagttgtataaaatctttcggtcagcattaacctaattggaacactaaata
ggtacatctaaaaaatataatcccccccaaaaatatgtagctcataagagataatgcattgaacacag
ataatattggcgttaaaaacagaactctaccacatttgcaacgaaatgtttatctgttcttcctacta
gaaaataataaaatagttctgcatgagcttgaactcgaagtattaggtgtacaaagaccttttagtga
atgaatgctagctgaaaagcaaattttaaatatgaaaaattagcaagacaaacatttgaatttgtggg
agatgagtaaaactcctataaaaatgaattgtttagtgttaaacagattgtgtatgaaatattaatgg
catattgtcctgagctccccttccgctgtttccatgtagatgactgaatttcaaacagaaatatgcca
ggaatgattacgtgaatgaatattactacatgagattgcttaaagagtatttcttcttttgccttctt
tttactttcgttatttcatttagtagttagaaaatactgtctacaaatatgtgagaactgcttaattt
atttttgagacattaattaattcaactaaactatattgactgtgtgagagagattcccttggtgaata
tgtggatttttgcggtggtaagaactctcctctggagcgcaaatggtattgctctaggaataaagcat
atacctcaggcccagatgaaccagtgcaatctacagtaacaggttcaaagatgacctcatgacctact
gtggactaataaaaatcaaggagacctactgcaaaggtttctgggaaattctttttctcttgcgttga
actaagtaatatacatatgtgatagttagagctgcagcctttgtaataccatgacagaagataacctg
aaataaggctgacagacacaagagggagacctaagagtactgagagatatggagcaggaccccctgat
tgaacttcacttgcagcccccttctgcagttttcaatgacgtgaaccagtagaatccctttgtttact
gtttttgattaatttgagtgcagctttatgttatgagcaactaatagcatcctcactgtcacaactgc
cctctatacggcaggcactttgtgatactaaagaaagcagtatacagagtagagcccagtgaataaca
gggcagatgttgcaattaaactgcctgtttaaattctagctcttccactagctaacttgtgactatct
aagtaatttaaccttcctataatcatacctatcttgaagacttgttgtaagatttaaagcacaacagt
gctactataaaacaggtatacagtaaagcttagctacttttttattaggccatatgatatcatttcat
taaaatcttatagccatgctataaggtattatgatcctcaatttataaataagacagctcaagttttg
gtcaagtgactttaccaaggtcatagagctagaaaataatgattccaagttacaagccaaacctcttc
aatgccaaatttacatcatcccccattacttgaagtgtaagattcacatggacagaaatttttgactg
tttgatcactgctatctccttatcatctaaaacagtctctggtccatattaggtgttcaataaatatt
tgtagagtacataatttccttcacagactccacaatctggtgaaggaggcagacatgtaagagaatta
tttcaggattccacagttgatgctgtaacagagctaaatataatgaatggaggaggaatgaataagtt
tgtctgggagcaatgctatggctattgaaataagtcttgctcatgctttgattgaaatggtggatata
gatcacacaacaaataacaattagataacagcttgttgggagaaagcgaggatcagtgtttgccataa
acatttctcatagctaatgtcaggtgtttgatttctcaacattttatatctttgactttgattttctc
tgtttttattttttaactccattctcaagaagtctgcacataagagtttcaacatctagcacttcata
actccgtcatctcctctcaggcttagagcaaattctgagacgtggatttatcgtcgagtgatttcttc
ctggcattttatctctgagaccaggatctggttgctaagcatgtagacatagaaatgcatttcttcat
tgaaccccataggttcaaactagtggataatgagcacaatgtcaatgtgattatttgtaatgggggaa
aggttaccggagaatattacacgaccatccacatagactaacattttcctcatgactaagtttactta
gcaaaacaaattaaaaacagaagtttgtttagcagcacagaattgaaggaagacaaccagatggttat
gaggaagattcatccaaactatgccagaactgaaagaaattaagttcattcagtacaagaattgtcta
gaataagagaatccattttgtgtcagcacttcccaagttcttgttaatgctaccttaagttcaattca
aaccaggcagcatttattacgtgttgtgctgggtcctaggaggaccgcgttttaagaacttactgtga
tcttctagatcaagtttttatttcaatatttctacctcatttctgattcttaggtgttccttatttcc
caatttatcccctgcagaaattgaggcaataagatgtctatcttattgcctatggtgttgattattta
tgttatattctgttttgtgaagtttgacctctacctaattaaattacattttcaattgtatcttggat
tgatttattcaataagtattctttaatatttttgcatgaggtcggtcaggtttcatcagacattagga
attaattataaaaatctctagattggtacttggagcttaaaggaataaggtggtggaacgttaaatga
ggaggaaagaaccagcagagctgggataaaattcatctctatcatcttcccacctgcttgatctctgg
catataatttactatccgtgaacctcaggtttctcttcagaaaagctgcagggttgttgggggaaata
aggcaattcctgggcttcagtatgttcaaaacagagcattaatattattatagacttttgatgattta
cacaattttagctttttggcaagacatatttactagtactaagtaaaagcacgttgactttctaaaat
gaaaatgtgtatgtgaggatgaagaaaaagaaagtgttttgtttgataatatagcattataacactgc
acaaaaaaaaaatggtatatgcagagacttccatcacttgcttatgatgccgcattgggatctcatta
ataagacacttcctcagacacttcctttgtgttcaataaatttcaatttcctcctttccttcagttca
cttcaagaaggacggcagcaactttcttgttgccaaacctgacaaatgttttttagtgctgattatac
tcgagcattctgtagcaaaatgctgtgggtgaaaatgccttccttcttaagggaatttagcttctgta
gtaccagaatctccttgttgaatgaacatgtactgcctaagtcttagtaatccctcctttttgagccc
attttctggcatctctccctttaatattcctcaaaaagttggatttttcctggacttttcatattaca
gactttcctttggtcatcctcatccattccgtgattccaactacattttccctccatcctggcatctt
ctttcttccagacttgtatatgcaactgcttccattcatacacttgaccaaccttttaatttctataa
gatcaaaaactcagctcacaagctttcccctaccatcgagcggggttcttcttttgcttctttgtttc
agacaatggcaccaccatactcgagtaaggcacgttcatttatcaggtcctaccaaatctacaataaa
ctctcttgaatttatccacttgttttcatttgaacagtcatttctttacctgggtagcctgcaccttc
tacctgcattgattcagcagtctcttcaccactggctctccctccctctcctgcctctcttcttgctc
cttcaatttattctctactcttcatagtgacttttattaatgcaaatatgaccttataactcccttgc
ttaaagacccactcatgtttgtctttgtatccataacttccggcctagggcttaacgcatagcaggtg
ctcagtaaatctgtggtagatgaaagaacaagttgtataaatactgaatggtctgatgtgctctttgt
tgtgtcaagaaggacattttgcagtcaggatagctacatcagtcctttagtaggcatttgacagcact
cgcattattcctcaagagaagatggatgtattgattctgtatttcaaatgacataacttttgtgaaat
aagaggctgccacggtaatctgagggatctctcaagttcaagggactccacagtgctttgtgtaaggt
aacaggctaaagggttcagtcttaaactttcttaagactgtagttcagggttcctatggtggggctat
aaccctgaattacatcctctttcatttcatgctgataatgagaactacaaaccaaggggtattaggaa
agaatccaggtttgatgcagggaaaaataaaaacaactgataatctctagtgtccccaacttcaagaa
ttcctttcttctttacaccaagctttttttctctgccaggacttactttgtcttctacatgtttaagg
gagaaaaatgagttaacagaaggggaggtacagcatttctatttacttagatgctagagaacaggatg
aaaggtatgaaaaatatgaaagtctctctctctctctctccccagccttcccccgcttctctctctct
ctctctctctctgtgtgtgtgtgtgtgtgtgcacgtgcgtgtgtgtgtgtgtcataatactcaacctt
tcttttctttcaagcatatgttgtggcagagacaagtgtacatcaaaattcgtggtccctctttcata
gtatagagttcttgctaggatccagctgcaagccagcaactacatttcccagccccactggcatctag
ttagagccatgtgactagttgtgaccaattgaatgtgagtgggagttatgttgcaggcataccttttc
catcttcttacttcccatttgctaaccttatggaaaagagtcccaaagacctaggagatgaaaaagcc
taaaatggaaggactcagagtccctgaattactgggtagagaaaagctgtttgcagatgggaatgccc
attttgtagtattctttcttttcttaagccactaaaattgtgggatctctttgttatagctactggca
ttaacctcttacgtatacatacagctatgtgctacaaagaggaatagatacattttttaatcgttgaa
aggggagaaagaaacatatttaggaggaaaataatttagtctctacaattgaaaagtgttttatgaat
aatattttgttttggcagcatattaaatctcaggcagctgaactacattaattttcaattctctatat
atgtttttgtcttcagggtttagtaacactgatatataacagtttctttcttttaatttccaaattta
aatgtctaagtttgccttctaggcagaaattaagtcccattgtggaatgagattggatcaacacttca
ccaagatcattttagttctttgtaatcttaaatgaaataagctaataaagcattaaattagcatgttg
taaaacttcgtgaagttttaatatgcttctaagtggcagctcttagcttattatctctaaagctaaag
tcaaaataaatgtctcagttgatgaaatggagatgaggcaacattttatcaaatttaacaaaatattt
tatatctgaattataaagtccagattatctagtaattatcatataaatgtatttaaccagacatgcat
ttttctctaatcagtagccctggagtctttggaccacaaatgtgccttatctcaaatgctttaactgt
gacattttgctttagactagctcgactacttctacagaaattatacacttcattcacattcatccaga
tgaaaaaaatacatgtagaaatgatcataataagtaacatttgtttaggatttcagagtttacgaagg
gtttttctattcactttctcacttgttcttcatgtaaactggtttggtggacaactgtcattatccct
gttacctggagcccctgggtcttagggagacttcttgacttctcaaggtcatgaaggtgctaactctg
accgtgtttttattcctactgtgccacacttctcaggtaaaaatcatattgcagacactttaagagaa
gtacttaagaaaataaattcctccagagaattacatttaagttgtttcattaactgcagtgcataaag
aaaggaaaagtgttcccaaacccatgtagtattttgctattgcttatggtaatattctgcacacctaa
tattgtcagcataattttccatgtaacaaaatgtcctaaatcagcaatgtccaatataactttgtgtg
atgataaaaatgttctgtctctgtgctgtccaatacaacagccactagatacacatgactactgagca
atggtaatatggccagggacactaaggaactaaatttttatttaatattaaataacgtttaaatttca
aaagccgcatgcggctagtggttgtcatcagatactgcagttatagaaaattagaatttacctcttta
aatactaaacctatttttaatagtaggatttttaaattaaaatagttctaagtgcttttaagtgatac
gaagtcaaatgcaagatttctgttttaatagtactctcaacccagagacaatcttcatgcatccttat
acatgttctttgttgccttattctagttttattttaacattaaatgcctctgttctacttgatattga
cttgcttcagagaacaccaagtatagtggaaagaaacacacacatgaggacttgaggctaccaaccag
gttcaactaaatgcactctgatttaattgtagtattgggatcccctgttgcatttattgaagaagaaa
aaaactttgcaaccaaaaagatatttgaaagcaactgttcttcttggacacatgatccctcataaagt
ggggcttcctgcttttcagagacttaatttctgttcatattcatttcagcaatagtaataatgatgat
ggcgatgatgataataatcatgatgatgcctaagtgttgtagtaatgcttcttctgagccagacgtta
gtcaaattactttctctacattaattcaggcaatcatcacaacaatcccacaggacaggttttattat
tatacttatttagctagcaaatgatataactaggttaagttacttgcccaaggtcatactgccaagac
agtggctctagtgtccctgcttctgaccatatgttatgctgcctatcctagagcttttctcttctaaa
atagtaaaataatatattctttgtttgtttcatacttttttttttttttttttttttgagagggagtt
tcgctctttcgcccaggctggagtgaggtggcgcaatctcagctgactgtaacctctgcccccaccag
gttcgagtgattcccctgcctcagcctccgaagtacctgggataataggtgcccaccaccatgcctgg
ctaatttttgtgttttcagtagagacagggcttcaccatgttgaccaggctggtctcgagttcctcag
ctctggcagtccgcccgccttggcctcccacagtgctgggattacatgcatgagccactacacccggc
ccatacataaatattttaagcgaagtacacatgcatgatcatcatacttttaataatttcatttaact
gtttccaaagaatgttagtatgaggttttctttttttctttttataatttcaacttttattttagatt
cagcgggtacatgttccctggatatagtgcatgatgatgaggtttgctatatgaatgatcccaccacc
caggtagcgagcatggtaaccactagttcttcaacccttgcctgttcccttcctccctccttcctctg
tagtccccagtgtctattgttcctgtctttatgtccatgtgcactcaatgtttagctcccacttttaa
gcgagaacatgcagtactcgttgtctgttcctgcgttaacgtgcttaggatagtggcctccaattgca
tccatgttgttgcacaggccatgattttgttagtttttatggctgtgtagtattccatggtgtatacg
cgccacattctttatcctgtccaccattaatgggcacctaggttgattgcatgtctttgccattgtga
atagtgctgtgatgttatatgtactttttggtatattcaaagagaaatgctattttcctcttgacata
tttatgtcaatttaacatatttatgtcccttttctttttaggagcaccattctcttcctttaacatta
taaataaaatattttttgcttttctgtttttgtaagtgcagttttattgacagagtgagacatacacg
tcgatattgtgactagctgcatgtcttctattatttagaggtctcactcaaatgtagattatcaaatt
ctgttagtgaagagggtagaacagcagaactaatgctggtttccttctctagcattatttgatgataa
actaagatgataataccccccaggtcttagatacctgcagtaggacaggcaccctacatttaatgctc
ctaggaatccttcaaagtgatagcatagttattatacagtaattgagaaaactgatgttcataagtta
gaaatttttccgaagttgcaaagaaagtgaatggaagaattataccaagttctggccgggcgcagtag
ctcatgcctgtaatctcagcgcttcaggaggccgaggcgggcggatcatgaggtcaagagattgagac
catcctggccaacatggtgagaccccgtctttactaaaaatagtaaaattagctgggcgtggtggcac
gcacctgtaatctcagctactcgggaggctgaggtaggagaatcacttgaacccgggaggcggagttt
gcagtgagccgagatcgtgccattgcactccagcctgggcgacaagagcaaaactccgtctcagaaga
aaaaaaaaaaaaaaaaaagaggattataccgagttctctttgattccaagcccaaacaaatccttttt
tgcaatatatgacattgtttccctgtttgcattccccattctgtgtatcacacatcctgtggcctgat
caaaattcattttcagattctgaatttattttccattgaatctatataaactataaagacagaagata
tatgtatgtgtgtatacccacgtttctcttccagtgtcaactgataaaaatagatttcaaagtctcaa
taacctttaattccctttttctcttaaaaattctttagaacttgtacatgacattctgactctagcag
attttagaaaacagagaggccattagatattcataccttactattcagatgaagtattcaatgctaaa
ttatgtaatttatctgctttgcaaattgtatggtcagattgagttccacaaaggagagataattttta
atataggcattctgtagcttccctaattattgaattagtttagagcaaaatccttaaattgtatcgtt
gctatgctcaaattttgtatacttgtccacgtaggctatattaagatttcattgaattttggtttctt
tctcagtgataattcaatatatcaactcaccactcagatttgcctttgggaaaatccaggcccctttt
ctggatttttagagcagattttaaaaaagtgattctgtatatgtgttgaaattaaccacatctcattg
cttttgaatgattgaggtaatgtatacctactactttaaaaaaaatgacttacttagaaggtgtccat
agttttataagttccattgaactggtttatattgtatttagaaaggaaaactactccttttatcctta
agggtgaaaacctggattttattatacaattaacacatatttattttttattatgaaatatatcacaa
tataaacgtttacagggagtgtttaaagtggtgttgtccaatggaaatataatgtgagtcaaatacgt
agttttcaattttctactagccatattagaaaaagaaacagagaaattaatgtaataggatactttat
ttagcctagtatatccaaatcacaattatttaaatatgtaatcaatataaaaattactaattatgtat
ttaacctttttctttagtaagtctctgaaatctagtgtatattttacatttatggcacattgcaattt
gcattagtcacatttgaattgttcaatagccacaggtggctaatggctaccgtgttggacagcacagg
tttaaagaataatatgaacatctgtgttccaacattctgagtttcaaataagaagaacaccatcagta
ttttgggagaagctccctatgttaccccttgctaatcaccttccttccccccagagccaaaagtaacc
attatcttgaatttctagtaaacaatgctcattttttaaaaaacgtatgttcaacacctgtatttgta
tctttaaagagtagctagttttagtttgcctggatttgaactttatattaagggaaccaccccatctc
taatcttctctgtgaattcttttctctcaatactatgttttacatatttacgttcatcaatgtgcaac
tcattgtatgtatataacacaatgtatatattttacatgcgtatggacatttgggttgtttttatgtt
tttgttcatcacaaaccacaacacacatgtgttcttgtatatgttttatagtgcatgtttaaaaattt
ctcaacagtattcgctagtagtattgtcaggtcatagggtatgcacacataaatagaaatgattgatt
agctgcaatttgtagtgcacacatatttgctatgtaagtgatccatgtttaagactttaactgaattt
aaaaaatattttattggagccaatctaaatgagctaagggtttgtattgtttacataagcaaagatta
cacttactgggtcaattcggttgattaactttggatatataaaatatatagctagttgttaaatagat
ataattattaattggcattacttttgtttgtatataaaaatttcaaaatatccatgacttaagcaagg
taaacacccactgggtggcttaagcaacagaaatgtatttcttgcagttccggaagttgaacgtctaa
gattaaggtgatgacagggttggtttctggtgagtcctcccccattggcttgcagatagccgccttct
ccttcatgacctttcctctgtgtatgtgcatcccttgtagctgttcttccttttatgaggacattaga
cttattggattaaggtcctacccatatgaactcatttaaccttaattacccctttaaaggccctacct
ccacttgcaggggttaaaacttcaacatatgaatggggttgaggagacctacttcagtccataacagt
ttctatattctgaagatggtctttaattaactaaacagttaatgttactttactgggaatgtcttttg
gatgggggaataagctgatgatatgagaagggttggtgaatttctcataagtgtgaaatttgttgggc
cggcccagcatgattttcaatcaaatacgctttggggacaagtaggttgaatcactacgagaggttta
aaagaaagcaagttgtaattgcaacttttaattgaaagaaagacaggctttgttgatgtgccagcaag
actgataactggctttaacgtagatagtaaggcagcagattcaatccactgatcgtgatctactagtg
aatttcaaagccttatgcaatagaactacaaaccctttccttgcccaccttgcaggtggatccatagg
caaaatgaacatttgcaaaaaagccgctatgtttcagaatttgtgctagggctttaatatctataatt
tctccaaatcctcacaatttaagaattaattcaacttagccccatgaatagggtgaaaattctgagat
ttaacaaactaaaataagttatctgaagacagacaaatagaaagagttgagatattctatttgaatgt
aaaattttcaaaaagtagaatgacagcgtcaggaattacagtctcagtgttgaacacaagacttagga
acaaatttgctgcatgtaatttcattgagatgggacaaagtacagcatacgtaaggaagttttagaac
aaataagataattattttacgagctttgaaacatgtgtaagaaagatacgaataaaagtataatcaca
tttgactaaaacatgaataccttaaaactgaaaagcactgagattatcattatataattttgaatatt
ttaaaccacaatgctttgggagtgcactgtaatattttagaattggaattttaacttactggcttaaa
aagtaatgtactttgttttaaattcaaagattatcttgtaaattcagttcgatctattgaaaaaatta
taaaattcggcaagaagccaaagaagaacaattatgtagctcaagataattaaattttcatgtttggc
tttagaaatatattcgtcgtgacatagtacatggtaatctagtgagcccagacaagtagttttctctt
tttgtcaaagggaacaatttgatgcgtgttcaagttgcttaaataaaattttgtatgtgctttctcat
cacaagagaacaatatgatttttgaaattatttttactttataaaagaaaaaaaaaagccctcacaga
gaaaaaagaaaaaaatgatgatgtctttgaaaaacaaagttaatacagctttacatatatttgaccta
catcagggttaatatttttcaaggtgaaacattagatgctggaacttgcaaaaacaggcaatcctcct
ttagatgaaacggacactctaagggttaattcattcactgagacctattgtgaagtaagccctacaga
gactgaaaaagttaaatgcaactcacaaaagttgctagaagagtcatgatgttaaaataaaataagta
cacaatgtatgctgcaagtatacttagagccatgctaggtgcggttgagaagttcaatacaggtccaa
gataatagctgcttctcctatagaacatgtcttctcattggagggataagacctgtgtctatgaaaca
ggcgtaattacatagctctggaactatatatgccgaaataaatgagacagtaagtgttattgtactat
aaagaatgaagaaatcatgatgagaagtaacagttaatgaatgttttctagaaagagtaggatctgaa
ttggccttaggttgtaagcagagtttatagatagagtagtggtatgtcagagtcactctgggtgctta
aacatacaaatccccaagtctcacccaaatgtgtcttcagatgaaaggaaaaaacaaatgacttgagc
tcccccgcaaagaacacgggtggtatattgagcagccaaggagtgaccagagtggcaggcccatgttg
agggacaaaagaggacaattagaatatgattaatacaaatttacagtgggatgagttgttagcctgag
gagcttgaatgtgaacctctgtgcaaaaaggagtcattaaatacttttgaaaaaggtgggatgggaag
aaaatgacattctcaagacaattagatcgaacagtattaagcatgctgacttattaagttatgcacct
tgagagggtggaatgagggaaaagggtctttatctggagtaagacaggaagaagctaagctgtaattc
ttactggactgtaaattatgtgcagatatattatctgtcatgttcgtgggcgcattctcagtacatag
cacttgaaacaggtactcgataaattgtcaaatggatgcatggagtgatttccatgcaaaatctaata
ttgtatagtattagaagggggaaaaaagcatggcattatgctagcagaaatgtcatttggtattgagg
atgaaacattttcaacagtttgcaaagccatccactcaaacattctgtcactttccaataattttgaa
ggatgttctttctactictaccttattacacaatgagttgagtaagataaagaagtcatgtgcaacaa
aacagagggagattttctgaaaggcactacaccaggaagttgttgtactcttgcttcatcttgccatc
ttggatatacttctggcgctacctccaggccagttcctcgttacatatgtcatttacttcccacatgc
tagactcaccgagttaatcattttgctgcagttaacacattttagcagagtgtaggtttatgggtgag
aaggaaatcaatgatgtttcaatacagggttcttttcccatcccccttatttccacttagaactgtct
ctcaagtcttaatttgcctctaaacttttttcccagcttacattcttttctgaaaaatgcaacgacga
tgccaatgtttgttgacctgaaatacattgtaaaacattcataatactttgagcagagcttccaaact
cccatttgcctcttttatctcccttaccttggcccctttttgaaggcaatgtgatatttaatccgttt
ctattgatgcttcaaaattattgaaaaactggtaattgtatttttccctttacttatcagttgctagt
tgacaatgagtgtttgcccaaacaataaccaatcaaaaggtaaaaaggagattccagacatatctgag
aagaaattctttggaagaagcccgtaaatggaatgggaattcaaacaaagccgtttccaaaagaaata
ctaaatggtctctaaatgcaaaaggattgctccccaagcattttatgggagcataaaaagctcccaac
acattttatgacaatacttctactcaatgacttcttgtgttgacatatttgttgcactcgacgttagt
atttacagcttcttatcccaaatatttacttaactgaagccctgatgtttttaaaaacttttcatctg
tgtttaacagcccattttacagaaacttatttgtttcatcaggcagatatttactgagaacttgcaag
tgccatatattctaaaaatgctgatgataaaactgtgaacacaatagattctcatggtgcttatggtc
agggctagcacacacacttgtgaaatgatcactgatgatcaaaggcataaacactacatttggaagaa
ataccgagggatccagaagtatcttggaaacactagcaagtatagcagatggtgggattggtgcttca
aagaacttcttgtggaagatgttacgtatgtaccttctctgtgccaggcactgctaggaagtgctgga
gagaaaaagatgtgctagataccgcctctgtcctatgtgcttgtgctttgtggggaggtgagtaggat
aatcccagttctcatgcagtgtaatgagtaccatgacggaaatgcactccaagaactaggcagcatga
ccagagataggacatttgagaaagacttcactcgggtggtactatcttagtctgggtgctaaaataga
tgtgatagatgagtaagggtgacccggaagcaggagggaaagggaggggctttcagaacaacaagtgc
gaggacattaaggtgaaatagagtataatagtattcccagatccttgggattgttctccattaggcta
aaacaaaggtgttttctcttctttaagatttcatgactgcagattgcataacagaaggtcatttaata
gacctctaaactgaaggaattcttgaattaaatcacaacatatcttccatggccagagaaaccattgc
ctccttatgtcgacattactaacagcaccagcacctgctgctcaggccagcgggagggttgggtgttg
ctgcctaggtaatgctcaccaactgatgtcctgccatgagtagttttgccaagttccacaaaaaaaac
ttagtgttctatcagcatctaatgagaattacagtcattagttaaataaaagaactattagataagga
gcagaatgaacaacacacaatccatcagcttggtgaatggtatcagatggtttctgggtgctgggcag
ctgtgcatccaagtagacagggagaatatatatgtcctttgccttatgtacttgtttctctaatccaa
aggcacagcaatccgtggaagctgctatgataaggtgtttagtggtgaaaatgtcttgaaagccagta
gattattaaagtgatgtttttaaaaatgcagatggagagtaagtactttttatctagagtagtagttc
tcaaagggaggtcccgggatcagcagcgttagcatcacttgggaacttagacctgcatgggccccatt
ccagatctcacttgaaaactctagggggtgtagcccggcagtctttgttgtgaccagctctccagggg
gttctgacactccaaatgttcaagtttcagaacgctactcacaggccatcatgctcggcatcacctga
aagcttgttagaactagaaagtcttggccccaccccaagcctactaaatcagagtttttgggagtagg
gccaagaaaactgtgggttaacaaggtctccaagtgattcttattcatgtcaaaatttgaaaagcgtc
gatcgaactgttggttctcagctttgattgcgtatctgaatcacctggggagacagttgagctattcc
gggcccagatcacatctagaccaattgaatcagaatctatggaggcaggacccagacatcagtatttt
aaaatatttcttgaatgatcccagagtgtagctaaggttgagaaacactgttctaggattaaaggatt
aatgtgtttgagagtatgttaagatcttaggcaaatcacaagggtgttaagaactaccatcttcgcaa
aaggagaatgtgcctcagatattctggtactgctttgattttaccttcagtagtcttacctattttga
gtatgcttagtagtactaatatgaggcttattactaatatgttaaaatttgtcttttaattaagtggg
tctaaacgttttaatctttaatctctgacccaactagaacttttctaaacattttcataatagtctcc
accttgtcttctgaccttcacttatgttctttcagggttcttcgtgtgttactagtaatagtaatggc
aagtgtttattgaacacttactatgtgaagattctaactggcttttaataatcacatcagctctggga
ggtagaaggtagggatcctccttgcttatcaggtgagaaaactgtactatagagaagttagcaacttt
tcccaggtcataatatgtgacagctaaagggagcataatggttggaataaaataaatctactctagtt
gtaccgaaggctcatatttgtctcacgtacttgatttggtcgaggcccaaggggtcaatttccaatgc
ttggattcctggatatgtagagttgtattaaaaatgctaaaaacctattatgtatcatacaatcatac
atatcacctaaagtattatggaaatgaatctgtattattaagggaaaaaggcctgtgtgaagaacaac
tgaaacttcattttaattgaaattaaataacatgcatcatacactaaaagtgcacgttatgaccccat
gaattacttcaggtggctttgattcatgttacatacactaacaaatatagaagagtgatataatgctt
cttaattaactactaatggaagtttactatttaactgcttcttatgtaagaatgtaaatgttttctga
aatatcagaacttttcattaggaagcacttttaaaaatagcaaaactgatatgcactatgatttccat
atacattaaattgaacttgtaaatgatgttataaattatagaaaccaaggggatgttcaaattagata
tttgtctaaataaatcatgtatggattgaacaaatactcattgagaaataaatgtattccttttcttt
caattatctaggattccttgtttatctcttcagaagcaaaatgtcttctgtccgttttatttccagtt
aaacattcttcagattatgtaaataagttaacttccaatcctcttatttctgtttatctcaccactct
tctaatttagacgtgatcaatatcttatctttttgcatttcatagacatcaggatccagaataattga
gtgagctcaaaacaacaatggcaagaatgatgttttcagaaaactcagcaatcattcgtttaataaat
attcattgcctaccaactataagcaaagtattggctaggccatgtggggtatacaaaaatgtattaaa
tatggctcattctccctaagaacttacacctattagacaaagtacatgcataaaaattataatgtata
atagaaaataaatacaagccctagaatgcacagttgaagtacgatttgcatttattataaaaagaaag
atgaattggctgggcacggtggctcacgcctgtaatcccagcactttgggaggccaaggtgggcagat
cacgaggtcaggagttcgagaccatcctggccaacatggtgaaactctatctctactaaatatacaaa
aattagccgggtttggtggtatgcacctgtaatcccagctacttagcaggctgaggcaggagaattgt
ttgaacctgggaggtggaggttgcagtgagccaagatctggccattgcactccagcctgggcaacagc
aagattccatctcaaaaaaaaaaaaaggaaagaaaagaaaagattaatttcctgttagctaaatcaag
gaaggcttcatggagaaaaaaatatttcaacacacacttgacgtagcagtgggatcaggctgatgtta
gggaagaatgaatgacattctacactgagaaagagatattcagtatatatatgaagagcagtagagaa
actaacaagtggaaatagactcaatttacaatacttgcctgcctggagtactctatacgttgactgta
agttgcagtttactcagaacaatcccactttctacttgtttatcctatgtaatcatttattgggcctc
cttttgctctcaaaaatatccttgtttggataatagattatcactctgttcctaaatgaactgccctg
tgtcctatcccagtaaaagggtgcattcgggcccttcgtaactgcctccactacatggttgattgaaa
ccagagcttggcattaagaagttagctgaacaatcagatttctattcttggaaaacccaagaatttca
gaatagatacagaagctgtatagctttaataacatgacagagttgtagccttgaaagctatgtacaat
tcagaattatgagggagaagaaattgaagaaacagtagcagccgggtaaatgcagaaacaaatgaggg
agacacctagggggtgactgaggcacaataatggaagagaagtgcagtgaaattgcttgaactcttac
tgatgagatttctactgttgccttgaatccaggaccacctatatgttcattctttgtcatgctcagag
ttatgacagatgctgttattgaattccccagagactcccttatcgtctcacctcaaaccttacaataa
tcccttctatctttctatccatccaagctggcttaagtaaagtctatgatccatattcctagtaaaca
gagaagggaaagagactgaaggcaaaggccccaattagtaggctattgcaatatttcagggaaaaggc
aatggccatcacattgttgtcccaggaatgagaatagaaatgaaagaagataatgaaagttgaaagga
ctgggggggcttgacaactgtttagacttgaggagtcagataaaataggaagccaaagataattcaga
atattttgattttgattttcatcaccaaataagatagtagtactatgaagaaaaaatggttaaaaaac
aataataataaagagaactcctccaaatagtaccaagggagggagtttaatagaggaaattaattccg
taggtgatgagagtcctgagaagccaaacgagaaaagatcaaaacaacccagggattggcagtcgcag
gaagctgttctcacttatggctggggctttaagcacaaggtgacatgagatttcagaatttgaagtcg
tctggaggcagctaggatcaggtggggcctgtcctgttcggcaggacctgcaaccacaggaggaggat
gcgtcaagcagaaagttggaacacaagaggggattcagccataagccacaaaataccttccagagcag
agagaaggagaaataccctgaattccgtattttccctgccatttagttccctgctattgccacacatt
gacgtattccatccagagaagtccattggcatatgagtctgggaaatgtagttcccagggggacatga
tcttaagggaaatagacaatgactggtgcaacaactgacctgtgtgaggcaggagggaaaaaacagga
ataatatagtttttctctagatcccttcatgcacaaagatgcaaaagaaatgtgttggcttaatgagc
cattctgggtggccctgtaggtggctgtcctacgaataagatttttagacaaaacagagatgacttca
aatgtcacaagaaaagtatcagacaggaattaatattgacttgatctgtcacaggcgtcaatgatttg
cattaagccaacgatcttcattgttaatgtctgggaaattgccagcagcattacgactacttgtgtgg
attagtgtaacggattcccccactaacattcaggaaatcatgtcaagcacagagtgcctatgtaagag
tggttgtgtctattcactacatttcttggactaataacacacttagccttcctgaattgccaacatgt
acaaaaccagattggggttttttagttgttcatggaactatcatttattgggtagctcctgtagaagc
aagatacagaaactctaattaggaataagacagtccctgtacttcaaagagctctcaggggaggcaca
caagtaaacaagcaattattatcatacgttaggataataccgtcatggtgataaccactgagtgatag
ccaaacacatggaagaggtacccaagtctaacttggggtagtcagagactgctttcaaggatatccga
gtaagtgttagctaagacatgatacgtatttctaggagggaaattttcaaggcaaggtggagattgtg
cagtgacgcccagagcctggattattttggtgactgctagtatttcagaatgacttcagcaaaagttg
tagagaagatagaagacaacaaagtataagcagaggccagataatgaggacctggaacagtggtttgc
tggtaaatgtttaacaagaggctcttggcggggagagagagtgtctgatttgcagcatttggcaaatt
ttgttgcacaaatgctccagcatagccaatttcaagctaccagtgtgacgtcattgaatgcagaattg
gaaagaaacgggcagtagcacagcattgtatagttattttcattacccagatataatagataaaatat
ccagatggtatttaatagatatggatgcaaaatttaaatatatgtacattcatgtgcttcatgttact
gaatgcgcacaacattcattatccattcattcacgtgttaatttaacaaacatttctgagcctctgct
ctgtgccaaacgcagttctagctgctggaattacagcactgaaaaaaaaaatttgtcctcactgaggt
aagacaaacattattatgcccattttacagctgagaaattaagacatatgaggattaagcagtatagt
taaaatcacacaattggtacatgaaggaatcaaagaggaaatcagctctcagattttaaatccaggga
ctcgtttctgctataccatactacctacctagttgagctggattttatcatggtttccctatttttat
caccatgtggttggataagtaaaataaatatatgtgacctttcaaataaatttgggtcatttttcttg
gaagctcatctggtgtgaactttaaaatactgcaattaataatgattataataccctggaactctgta
gcaacctcttttgaagaactccaaggagcctctaaatgtatcaaactaagttcttcaagtgaattagt
tatcatctgagagtaatatagacttttaaaaatgcattaattgtattaaccctttcaggcccatagac
ttaagtgtttctttctccaaataaaaatagtaatctctgtccattttctttagagaataatgaagtaa
ttttcattgaatatgtagtcaacataattacttcaattcaatcgtgaaggattttaaaaattatttat
gtctactaacttaaagacatgcatagatttcaagaacttaaaaatgcatattgcctctttgccctatg
cctcataaaacaaaattatgataacgttgtgtgttacagaaaaacgcactgattgtaatgaagggtgc
ttcaaaggccatgaacttggaaagcaacttatttacagagacccccagcaatagcagctaaaagattg
actgactccctttattttcagttatccttcagacacttttgacctcttcctgtgcctttctagtcatg
tgcaatcttgtggatatctcttccttcctcttgttattttctatttcctctgtttctatttgtttcta
aaaataatcatgtttgaatataggattagcttccttcccatctccccattaccaatctctcactatac
cgctatgttattaatcttcctgagaaatatatcaggttcattacattagttaccagctcaaaacgtat
cagtggctttctagtcctcacaggctcaagttaatctgcatattctgactttcatattctgggttcat
gcaaacttttcaactttccctcttatacctacttaggaggaccctcaggttccatcatgctcatgttt
caagccagaagttctcctgcctcttcctctatgtagactccacatagactatgatatcctgcttctct
tttaatcctccatcttcagctcacagccacactcctctgtgaacagttaaatgattctcccacctctt
acctcctatagcacttatttttcatgcagcatttttgagacttaattaaatctacagttttaaaaaat
gtttttctaccacagtctcttattcatactaaaactttcaagtctatccattttgcttatacaaccac
accgttaggtcttttaggtccaagaatacaagagaatggcaaagcacgttgtttacatccacacatac
tgtgtaaattcaggtaattttttttaatcctatgatcctcaattacctcacctgtaaaataggtacta
ctcatactgcagaactcttgttggaattaaataaatgagtgtattaaaaatgctcaacaagatttggc
acaaaatcggtactcagtaaatgctaatcattattccctttctcttcaaagctccacaattctgtatt
catatcaccctctttatatcatttgcaaaaatgtatcctattccaactctttccacctagcctcaaca
tttacaaacactcctggtgggaagggaaagcttttgaggagagcacatctatactcatttacttctca
gggatgcaagctgccctgcttactgagggcatatcttcatagtcacaccggagcccactgtcccctta
tactctcaaatgggcagtagcaaatcatcttgatcggtagtaatgacctgtctctaaattttcacatg
catcagataatttcttttttagtaagtgttatcttacatatatgccaaaatatcaccattatatggaa
cactagctgaaagaaaaattattcagtagtcttaattttctagctaacataaattctctccattttca
tcatccatttagattaaagactttactgttagctgaatattcagagactttattctgatttttaaaat
ttatgaggttcataatgttaagacttcaagggtgagctgtttgtgtcatttataatgcgtgactagac
agtaactagaaaatggattgttgactttacaagatttctccccaccacgtccccccaaacctgtgctg
ctgtgtatttggcctgaaatctttacttctagtcaatctttggacctaaagcctaccagcttttagca
tcctttaagattgacgtgtctctgggagaccaatagatgctaaaccaaatttcgtatgcacttggcaa
tataggataataacaaccatactccctgcaattgtttcctaacacagatgtaacaaattaccacaagc
tgggtggcttaatagacatttattctctcacaaatctggaagctaggtgtccaaaatcaaggtcaatt
atccctctgaaggctctggggaagaattcttccttgcctcttccagcttctggtagccccaggtgttc
cttgatttcaagcagcacaagttcaacatctgctcctgacctcacataaccctcttctttgtgtgtct
ttctgtgtccactcttttctttattattattattattattattattattattattatactttaagttt
tagggtacatgtgcacaatgtgcaggttagttacatatgtatgcatgtgccatgctggtgtgctgcac
ccattagctcatcatttagcattaggtatatctcctaatgctatccctccccccctccccccacccca
caacagtccccagagtgtgatgttcccattcctgtgtccatgtgttctcattgttcaattcccacctg
tgagtgagagtatgcagtgtttggttttttgttcttgcgatagtttactgagaatgatgatttccaat
ttcatccatgtctctacaaagaacatgaactcatcatttttttatggctgcatagtattccatggtgt
atatgtgccacattttcttaatccagtctatcattgttggacatttgggttggttccaagtctttgct
attgtgaatagtgccgcaaaaggacaccagtctttggatttagagcccaccctaaattcatggtgatg
tcattttgaaattcttaactaattacatcttcaaagaccctatttccaaatctggtgacattcaaggt
ttcagggacatgtgactattcaggggaaactattcatcccaccacatcccccttgaaaattctggaaa
atgtagtaataaaggcttctgataaattagtgtggaaagtattcacggttataaattactaaaaagtc
tcactgtgagctcttaatcaaaaggccctataaaacatttatttgcttgattaaaactacacatccga
tattttggttttggatttattattatttttagacttggaataactattttatgtgaaatagattccat
aactgaagcagcatacctctcaatttcccaacatttattttattattttttgtcttcacactacttaa
taactgaggaaaaatcatttagaccaaagttcaccttggttgacaccatccagacagctacaggaaat
aacaatggaaactaaatctctaagaaaaagagtctttcatgtgaaatattgcagagttgattctagat
atatagctgttggaagaatggatactattacatagatatggcagagtggtatccagcacctttcaaca
aagatctttcagagtcagtcttattatgtctggagaatttacccagggcttaggtgcttttactgaca
atctaaccacctgcaccccacccaccgtctaaagctaaagtttattggaagacttaggaaatcagtct
tcggaatgtttctgagactggtacacccaccacttcattaaagtgcttcacttcacttcattagacaa
gaagtaaaatacttgtcaggaaattatttatagtaccatgtatatgggtatcttatttaatactactt
aatgatggtactacaagttatataaaatggagaaataagtcatcaagtttgacaataatgatatttga
tattatcattatcttttttattcgttcccacagaagtactctgttattggtttagaaaaatgatattt
gatataataaagaaggaaaaggtggtaatattctttattttttgtatctttataccccagctctttca
ccaatctcccccatctctgtagttctcctctggtgtccccaggcagtgaactattcccagtggttagg
gaacatctcattgagtaagttacatcaacatttcttcacatttcaggacaacaggaacagtgccaaat
cctagcccattgttcaactctcaagccttattatcctaataacacatccatcccaagaaagaattcat
caagatcagagaggaatacgtataattttttatagtacagtatttaaaatgaaacagcttttggcccg
cgtggtctcagtgggctcaagggggaaattcaggatgctagctcatctcacaccaagtttaataaagg
gtgtcctataaaaagctaatttcttgctggtaaattgctttttaagtaatccttgctgttgcaagaga
cccattcatagcgctgacactgggagccatgttggaaaggctagatatgctctgggagataaggtaag
atccaggtggaatcttctctttacagaatgacaatgtatatagctaatattgtcctttgaggctagtt
tgcatgcagttgctggtatggcactgctcagcagcctgctgcagataagaatgagtgatgatgcccta
gattttaatggaacttttagagtgcatgcagcagtggggtgcagtcttcagcaaagaaaaacgagctg
acttgcaggcatgagagatcatcaagaaagataaagaaataggacatccactctaggttaggcaaggc
tttttagaggatattatggaaatgagcaagaaccaatttaatttttataatgccactccatttaactt
taaaatacaaggtcaaggtactgtgtttttcataatgattaaagatttggagcactctttctgttgaa
acatactgcatctgtttggcagaaaaaaaaagtgacaaagaataaaactgggatcagagaacaacaaa
aacatattctgtcacttgcctaacacaagttaaaaagcaaaggaaaaagagacaactctgatggacat
gttcatccttatcccaacagaaggatttatttacctaaggtcctattatttcaagttactttgatccc
aggatggtaacataaaatgtacattttaaaataaaatggaagtataagatcaataaaaaccacatatc
tgtggataaaacagcagattcaatcttgtggctgaaagtttgctttaacccaacatttggtaaactat
tcactctgtaatttattaaaagacatactgttattataaaactatctcagtttgcatcttgttggttc
tgtcaaaatttcatcctgctaattctcaacttgtaatatctctgatatacatgattaatctattttag
gaataaaacaaaaactacctttatcttacgcatttctaggaagtgtttttagatgtaaagtaggggta
attgtagtatagtggaaaggattttgaacttgaagccagaacatatgtctctgccaaaaactaggtgt
gtgaccttaaataagttacttagcttcctgaatcttagtttgtttagcttttttctataaagtggcac
acctatccacatcacagttttgttgtcaaaattaaataaaatactatattagaaagaaacttttagaa
agaaatttataaactgaaatgtactatacaagtttaaatcattctcattattttcttaccctaaaatt
ttgaccttatttttcttagcaaatggctgaatctgtaaaatttaacccccacgcagcatctggattca
agagaactacggtcatttctttatacagaatactaattatacacatatagcaaaacacaagttttttc
caactactctgtgtttttaaagattcagtgtgggcagaaggaattttatcaactatgttaggggaaaa
aagtctgaagaaatgaaaataatgagaaaaagcactgttgatttaagtgcaggaacataaaacttcaa
ggcaaatgtgaggccaactgagttcatatatatcctcacaaaatgatttagttaatttaaaaactttt
ctaataagcaacacaggtaatcccaaattctatcttttatagctctaagagtccccataatttattca
gcaattatttaccacccacttattataagaaaagccctgggataagtcttgagaagaaactaacaaaa
acaaaacttgattgtttgctctcaaaaagctgggtctaaaataggcaaggtaagattttgttttgagg
agcccgtattttccagcactgtccattgtaacattaaaatagtttgccaaaatcctcactctgtgggt
gtatttgcctagggtgctaaaattgcttaaaaactttgttatttggctaactaaaatcactgaatagt
aaacagtagcattagagatggcagagacattaggtgtcatgcagttcaactgcttcacctagcagaca
aagacattaagttccatttcttaaatttaactatctggttgaggatacacagtagcagagctaaatca
agaacctcttggggttagagtttttgtttatgcattactttgttttggaattaaaaacagtgcctgtt
tgctaagttaaattgaaaatatgctctgaaggagaaaaacagctataaaaatagacttaacttccaaa
ctatggatcacaataaactaaagaaataatttctgtagcaataaactccaacactttccataggacca
gaaaggcttgagaaagaggagaacaaaaaaatgctttggggcttaccatatatatggagaaagctaaa
tgaataaaccagttgaaagacagcgagttatactagtaacaatattactgatatcggagctctcactt
ataaattgtatattatgatcatagtgactaggtactttatatctgctttctcattccttcctcacatt
aattcacatgtaggacagattacctcttctgtttctatccagaggcctagagctcaggccctcatcga
agacagacagagctatcatccttattctaaaaaaaaactaagaccccagacatagctgtgctacttat
agactagaatgtgagagaaaaagacaagctttcatcatgggcttaacaaactgaaacacttcttcaat
tttgagattgagaaacttagctaatgctaggtgtaaagatgatatgctaccttcataaccttggtgag
gagaaattagcatttctctcagtcctagaaggaggatgaccatgaaggtcttcattctcttgagaaga
taatcaaatgcttcactgccctgttaacggtttactcaatattcaccaagaaaagtagatgggattat
ttttgcagacacttatacgggtaatttattctgataagcagagacatacctttagtgcataaattgtt
ccctttgtgctctttgtaataaacatcaccatagagaacaaacacgaagtaatgacattgaattaaaa
gacaccatagaggcaacagcgactggaatttgtgaaagtaaaaggatagtgcaaacagttgtgcgttg
cattctgctctgaagattaacaagctgggtcaggctttgaccatcatgatgagcaggagatttttcta
atggaaatccccaatcaagttcctgctgcacccagaaaggaacggcttacagaaatcttacatttctt
tgcacataccaaattgcttggcatattctatcacaaggtttactttccagggaatgtgatcaagaaat
catgatcctaattcctagttaaccctcaaagtttctcagaacagtcagtgcatcactgtcaacttttg
tgcaatgtggaaatcagaattggtcacacgtttttccggccactgttttagattcatataatattagt
gaaatcatgtcagactggtatagccatgaatttatacttcatgaataggcactcaataaatagtggat
taaatcgaccgatttgatttttacctccaataatttcaaaaatatcattgaagacaaggttgttgaag
ctgtcacttttcttgctgaacctttgttgtgccaggaggaacagatggtaaaatcaaaagtgattaga
gaatcagtggggtgggggtgagattggaggggagaggtcttcccagtgagacccgctagcgtcttccc
tgagcagtatgttaacccaagacaattttagaaatctgtgcccctaagttgcttgacatccaaagcac
acttgatgcatcctacatttctaaatatttttattgttgtttctcggtagtaatcatctggtttagtc
actctaaaagtcaaggatgaaattttaaaatgcaaataaaagtgcctactttctctctttccaattcc
tttttgttttattgaggtataatttacatgcacaaaaaaatcgcctttttaaagtgtacagtttgatg
agttttgacaaacatatgcagtcctacaaccacgtccgtgatcagaataggaaatatttttatcactt
caaaaagtttccttgtactcccgttgcagtcagtctcctgccccaccccagcccctggaaaccactga
taggtaaaagcacttttaatctgaaaggtatttaatgtatggcagtgtcagtggtaataataacaaga
tttattcattggttcactgtatttttgagcacttatatgtgcccgttgtatgcaacccattatgctca
acccctgccctcctcaccagggataaactagtggcagagatagacaaagaagccgtctctctatcacc
cctatcttatagaacattcttcaatgttagaaatgcagtataatgtggccattgagaacttgaaatgt
gcttagtgggaatgaagaactgaagttttaactttatttaatttcaattaatttaaatttatatagcc
acatgtggctaatgactatcccactggaaagtacagcttctatacaatatgataatatgatacattat
aacgcaggagtttaaccaagtgctaaagctttactatcaccagggtcactggtgttatgtgaaaagaa
aacttacaatagaaaaataaatcctttaaatagtcacagacctgagaaagtttccttctcaagggaac
acacattggctcattcaaaggaggttaaaaactagcatttaaggtaatttcatgaagctttcctttgg
atttctcatgcttattgtatacataaataggcaattttcgatgggacctaataaatcactgtttttta
tttgaacattttaacaaaattatcaaacagcattgcatttatgttcaacctatttgttctgagaaaga
caacgattaagtagaagtcatcaaagttaccagaacaatttttgttcttatgttttagaaggcattga
aggtgtttaaaatgtacacttatagagtcagagtactatgcaactgtggcccttatagtttatccgtc
atgcatctaaagccattgttacatctgtttctaattgtgcatggattgtccaagatacacaattggaa
attccattttatttatcaatttgaagaggtttcacccatgtggtcactatgatcactatggagtcaca
ttaaattgagaagtctccagaagttgcagtatttatttaaaattctaactttcttcagaggaacaaat
tctccatttctggattctgaatcctcattagccataaggttgttgtaagaatttgcagctaataggaa
cacatcctggggagagaccagttgaaaagtaacttggttctgagtgaaattatacagagacagtttct
acttcaggtggtgttgctaatgaagctatcatggtaattttagcccatatgatccctaaacgacttca
gaaccacttttcatccactaagaacccacttcaaccactgccacgttcactaccacagtataatatgg
aacaccctctggaattcagtaagtaacttcttaactcattggctatagagctttgcctttgtaaattc
tttccttttgcagtaaaagagattgtttcaaagtaatccaattagtccctaggcatgtctagaaaggt
agagtcaacaacagtaaggtaatagtccttataagatatgtaagaaattatcagtcatttactttaaa
ataatttgtacacttttccttttatatggttcttctatgttgaagccagtggtcatccagtgattaag
attagccaaactcaaaaggctaaaactaaattcaaatggtattattttgctttaattttatgcaatgc
tatgtatttaaatttcatgaaagtttcgtatggcattgctatcaatttcagtcaggataaatttcccg
tgaaataatccacaattttcaactgtacgttgggtacaggtaaggaaacacccttaagagcttatcca
gttattagctggtattataaatttcaagtaattcaatgttcaattaataaacagttactttaaatggg
aaagtatgagtcaagagttagtacaaaggagaatcttaaaagatgaacatcaaagaatcttactattg
atttgttggtgcctttgcttgcacttctccaaattgacttgacgttttaaatttgtactgataatcat
cagagtcaaatctgcttttaggcaaaaagtatccgctagttattcccctactatgaaagtgatgagat
gaattgatcatgtctccagtgtatggatggatgtctttgaggaagacctactgaccttatgtttatct
tctgtcagcatggtgtgactatgtggagagacagtgctatttgctaaatactttgtttttcaaataaa
aagatttcacagattatgcattgtagaatttataagtattcttttatgtctttgaatgtgccaataca
atttttatgaagttggaactattttatctattttaatgaaattgtaagccttctgtgaattcttttat
taattttattctgaagaaaatctgaccaggttagggaaatcaggtcaggttacgacgtgatcccagtg
gaaaagctgaactgtggactgtgatttaaaatagggaagaggtactgaagtgttgtttttatttttgt
ttacaaatcagcctttctaactattatgtactcccatccttctatctttttctccaccagaacgtatt
aacaggcatgcatataattaatgcttttcttgagataatattaaaattaacttcatctgtcaggccgt
ctgggctaaaagtacacagtcagatctgggtaacatttgagttgatgtaaatatgcccacacatactg
acaatgcttaccatttattgtgtgaatgaaaagcagtgtaaatattgtttgttctactagggaagctc
cacattttaatcaaactttgaccgtatttctaaaatgccagagcatctggaattgttaaaggaactga
tagtttttgtgtttttaactgttaggatacttgaaatccaaagggtaaagaaactcagctgatttata
cgtttcttcctctttattttaatgtgataaaatgtagtttttgtcatgggctgacaaacagtggtaga
ctacactaactctgcgtttgctgggtttaatcttaccctctcaaggcatggaatgggagctcacttca
gacccagccatgcttcactgtccactgccttctcatggatatagtgtgaacattaattagatgaattc
cataaagtgctttaagctctttggagaaagatactcgctgcataattattcttaactcccatacgctc
ttatgatataaaccattctcccaggaaatcctttttagggattatcacttaaaatgaaattttcatta
ttaaaagcaggaagaatatacatctactgacagacgaaaatgtgcttaaggcgactgcttttaaatag
gcagaaatcctgaactatggagccatccatgcctgaaaatactgagtaataatgaaaactggtagcaa
atttggaatattaatcatcacattaagttgcaaagaaaaaaaaatacaagccacatgccctttaaaaa
tacgtgcacaaatctttattctagaaatatataactttaggcctaaaaaagtacaaaaagtaaattat
tttatggctctgaaagtatccttaatttactcaggtgacaacaattagtgtttaaagagttagttttc
aatcttagctacaagttggaattactctggaagctctaaaaaaacaaaaaacaaaaaaaaatagagat
gcctagttcccacctgcagaaattctgatttgatttttctggtgcgagacctgagaataggaattttt
ttaaagcttccctagtgattctagtgtgccacctaggttgccttaaggtaaacctcatattatgcaga
acctagcaatcacctatcctgattttatagacgaagatcataagacccaagagggcaaattgatttat
tcaagattgaatatacaaatgatagaagattcacataagatgcagtatacagagtggcttgtggattc
ttgccaatgcaggcagcagaattttctttagggttcacccagttcaggcacctctttgcagcagcact
tgactaaggttcttctgattggatcattatatgggcaaaaagaaaaagcttaattgaaaagagctgaa
cccacattgtggaatggaagatatacagtttacacgttataaatgattaatattcatgaaagcatact
gccctttcctcttcccttcccatagatgacatcattgcattggtgtagttaggttggtggtttcttgt
tgttgatcttggttctgacacagttcatcacttattatcctggcttattatctacttctacattcatt
gttcactcactcactaattaattcaacatggtttttattgttttggaccggttatatgcctgcaacgc
tacgtaaggctgaggatattacaatgaacaggaaacaaccctgaagtttaaggtatcaagcctttgag
ttactgtcttttatcatagctgatataaaattgaagccccactttttttgttttcaattactgaaaat
tcagtgctaaaaaaatgtggatttttattcaactagataaagtactacaattaggtttccactgacct
tggctgtttttgttcccagttgccattacataaatctgtgccactcacaacttaggaagggtgtaaca
ttctctgtaatagtttgcctttcgaatagtgtttggattcattactgtccctcgcagtttggaataat
gaccactgaataatcagtgtttggagactaaattagtgctgcaaaattccctcaaattacctactgtt
cttttccctgtcgatgtatcctcatattcactatgattaccctgagaagaaagatattgttgagaacc
actttacctactcgaagttttggtatttcaaagattcatacttatgtcatgttgattacattagcact
aatactattggcagaattctaattcacgttattttctttttttccaatttctctccatgcctatgtgt
tgtcccttcgcagctataaagccatggccgattcatgggtgcttttgttaaggcgttcagcagtcacg
tttgtagatttttgaatgggacttagagcccttttttgttctttatgtatttctctatttctcagcaa
aggaaatgcagacatgcaagaaatagtgatcaaatgtcctgtgtactattgtgggtgtcattaatggt
atagggagaaatagaaaatagttgcaaagatgcatttaacaaataaacgaggtcttgagattcaccat
gaatgtggccccttctatgaaaagtagttaacatccaactgcaaagttgtactggatcagtttgactt
taacctttagctaatatgaaaatatggaattgtgtggtggtgctcacaaaaaagaaaactcatttttc
ttaattatcatcaattaacatgtactgactacccatgagggaaagttaatttgctcttgagtggaacc
aagaaaaatagataaagcaatttctgattagccagtgaaagcctctaacataaaatttccaaagatgt
aagaaaaatagataaagcaatttctgattagccagtgaaagcctctaacataaaatttccaaagatgt
gccataaattatccacaaaatgtaaaacttttcaattttggtttgcattttcttttttcttattataa
aggtaataagtgctcattatagaatttgaaaaatataggaagttgcacggaagacgaataaaatcagc
cataatcctacaaacctattgacacttgtacatatgtttgttatctctaatgcattcattatgataat
gcatcttttcaaccaatagagtaatcactggtgactttcaaatttgcctactcatttttcactctgtg
gacttactttactacctcttgccctttttcagtaaatgaataaatatttaagtaagtaaatacaaatg
taataacttatgcgctcaagcacacagatacacacagagagaatttggaacttcggaaatgccatcct
ctccctagggccgcaagtgagttgataagcacgtaaggaaggataatcaggggagccttctcgtattg
cccagatggctcaaaattcgtcatctctaccaaacaactatttggagctttgaagaaatatccatgac
ccctttgaattcttcagtttctttcgcgttcactttgagaaccaagtgacaagtgaatttcctgactt
ggtcttttaaacctgttagcgcagttccattgagattttgtgggcacaagattgcaatgaagagatca
acagggagaaattcatttccctatatatgtgcgattaatccggagtgctaagggcagatataaagcag
gtgcctactcctgtataacttggaataaaaccatttccaaaggctgatgatcctcaagtcttgttctg
caaatgactgatgtataacttcaggccaatttttctccagttagtctgtgtcactgggagtcccattt
ctcggggagcagccccatgctttgtcaggtgcggagcccacagaaggttaatgcgaaaagaaggcctc
ttgccagactgttttccagatgatacgtagggttattagtttgagctccttaagaagatttttctcac
ctgtcctaccaacttatgtttatttcattggtgttagagggtttcagtggcggaagtaaaatatttag
cggggaagggacagcgttcatgggaattttgcctaacttaattttgtatctttagctcattcgtagtc
attgtactttgtgttttgtcaactgaattttgtttgcatacaaaggcacaaaatgtttgcttcagacc
tgtcactcttatttttagcatggttagacaaaaactgagatgctttaattgtctaacttatcccagtt
taagtgctgcaaaatctcccaggcaatgtcatgggcaactaagggataaaatcagagatttaaaggtg
ccaggtttcccacgcttctaacagttggcgttttgggtgtatacaatccctcagctttcttctttagt
ttatggagtcttgtggagggaatagcaggtttttagctaaaattatcatgctgtcgagttgggtctct
agtgcatcctgaagagcttgcattatttacagaggctgggctatcattttaaatcctgatgcttcaat
gcccgttatcattcttgacaaactcttccagcccgtggtctgttttcctctgtttgcttccatttact
ttcctgagcaaccagctgagcaaagatttacataacttttgtttaaacaaaccctgtacagttcactc
tttcagccagtatgtaaacacttttgagacacagttacatttttctattttagtcccagattctgttt
atttgctacattttttgtgcccacatttttgtctttgttaagtctcttacagattcacatgaaaaacc
agaaaccgtggctgctcaaaagtcattaataatgagatttttagctactgtttctgcttgtaaattct
tcatttcacataatacagtctcaaaaggccacagagaattcagcctcgcttatctctgtgttgcagat
gatggcttctagccttacccaatcccagtgcagcttgcttgccatccaggagtcgaatttgtttccat
ctgacattagcgtattaaaaagattggagatcaacaagcaacaatgttcttgtagaaaggtaatcaag
gtttagagcctgtgtgtcatgagactcctagcatttgaaaccgctaaggggttgaccaccattgtccc
aagcacctgtttaagattctttcctatgataagggacctaaagtgattagcatactgataagattttc
ctagaataacctatttatttcagtattattctttcaaatcttaattaccatcttttcctttacccagg
gtcttctttctacctctacgacacatttaattacctatattccccaacctgtaccatattaaattttg
aatggaagttttatagggtaatttattggaaggatggccttgagtgtcattatgttcaatgaatgccc
tattttgacaaagagatgactaaatgttattgaaatctttttaatccaccacgcttctgcttagatgt
aaatgcaaatctgttctttacatttgtgattgaattgaacttgaaaagtaccgccatattgattcctt
ctgcaaataaaatataattacatttccctaaactttctacactctcccaagagattggctggctttgt
attgtagatttttggtgatcacagaggacaatgcattatcataagaccaataagatttatttttacct
tggtaaagaattttaatttatttctagtttcattttcatttatatccatctcttctcaccctctgctc
tacaaaagtatatatgactatataaattgaaaaaaatatcaagtgcaaaattacagaaataaataatt
aggttattttagtggaggaaggtttgttgtgggtggaggaggagaggagtgagccaagaaaaacgagg
gaccatacgtgatcatatttttgcagctattttaaattgtttgtgtatatactttaaaatattataaa
ataaaattttaagtgcaatgcatatttggagccaatgatgagggataacttcagaaacgtagcatcat
catctagtgctttcatagtcctttcaacatttccagatagttttaatggcctgctcatggaggcaatg
ccctaattttaacatatctcttcacaactctgatttcttgcttcctaacattaaatgtcttcaaagct
tctttcaccactaattccttatcaagaggataagccagtttattctttaagaaaaactagctacacaa
aaccgtaagtcattccaacataaatccttcactatcctctctctatagatttggttttgattcctcct
gctgaaattcaaccttctttcttcagctatccacacgtcttaccctctaacttccctcaggagtgtct
attagctcccattacagtgaccacagtaatatagtaatcccctgctgttctcactctccacttcctta
cactgcgttttaagtctcttcatattctttatcaccttgtatcatgcatcggttttcttagttgttta
ttttatgttgccttcataaattccatgagagctcactgccgtatctttagaacatggaacagtgcttg
gaacataatgggcattccttaaatagctgtagaataaactttcaaaatcaacaataatgtatttgcca
aatccattggcttctctgccattttatcttgttcaataccactgcgatattccccttccttttttttt
ttttttaaagtctgtaaccctttagcttctgtaatattcctagttttttattcctctcatgtgtcaaa
atcatcagttgaggcttattgttttctctttctcactctgacctcacctttgtttacatctcatcttc
tggctttggctatcctgttttttatctctgttccaacctgtatttctagccctactacctggacatga
catgtggatatctccgtatgaccgcagtttccatatgactttgcaaattcatccctgctctcccctcc
aaagtcatccccacaattgacttcctgttccttccaacctattaaggttcaaacccacttttgctcct
cctttgcaggctacacttttccttctcagtacctcttttttttccaagttcttagataaaagtcatag
taccttacgttgtaattgccactggtctggtctttctgcctgctttcctttccatttgtaatcacatt
atccattccaatccatttataatactgtgatcagccataaaaataacatttatcatatcgtttgtctc
cttaaaacctgtagtagatcccctctatttacaagatctggtataaaatcacccttcctgatattcaa
tgcctgttttaatataatctcaatattatgcgtcataaatccccctgtgttcttgcactttttatttc
ttatacatctcatcaaccatgtcttatcaactctcaaaacctgtattcgttttcaggaaaactcataa
attattcttttgtagaccttttgtttgtcatctttgaagatctctctctgaactacaatattttgtct
gtataatcaatttggaaattcatcaggtattgaaatatgacatgtcttctattgtcttgaacattaat
taaaactttatttgactttttatatgcttacatcttgtttcctcacggagtgttaacctactagaaag
taatagtttaatcttatatttattttaattcagatttagtagcatactttacacgtggtaggatgtgt
aactgccttacaccttgcttacgtgagttattaatgttttcgtatatttaatctgaggatgtactagc
aatgttaaaactgtaccgcatgaaattgagtaattgaactatttgttttaaatgtgttgcttaactta
ttgtaccattttctcataatcacagctcaagttaactttgtggttgtacgtattatttcttgtgaaat
gccaacaaacttagagcaaggaaaataacaggtataatcatactataaaggcaaccttaacactagca
tagtctcttagctcatatggtaactacaataatgtacagtgacaaagagaatattgtactttcttagc
acacactttcctactactctactgttgtggataaaaacagacatactttaggagaaactatgttattt
ccaaataatgccttaaaggttactccaggaaaaggcatttacataaactatctaggaaaagaaccttt
taaataatataaagagctcacccaaaaggactgaagtgtttagttgaaaaaaagtaaaaatgtcgaag
actttgaaaaatagtttcttgcagtatattttcatcgcttccacttacgttatgaagacattaagcgc
tagtttatcaaaaactatttttgtacatgtcttctaatgacagaacaatgtcaacatgattttcatca
ttgagaatgcgtaaagaaaccctttgtacagttttttctatgaatgttcccctaagattaaagcaaat
ttccaacacgaattaggcactccgaaaggaggaggggagggaggggagcaagtgctgcaaaacttcct
gttgggtactatgttcactatctgggtgatggaatcaacagaagcccaaacctcagcatcacgcagta
tacccttgtaacaaaccaacacatgtacccctgagtctacattaaaaatagagattaaaaaaaggaaa
tcagtatataatctaataaatacctctcaagctttctcatttttaaaataaaattttagattattatt
ttaggaataaaataggctcttcattgtatataagttcatttctgagttgcaaaaatcctctctttatg
tttttttccccgtattagcatgtttttctcctgtttttccccactcaacttggctgccacaatcagaa
agcacaaagacaattttttcttgcgcttgtaaatcaaaaccttagcatcagacaaaataactgctcca
ggtctgtcaaatagattcatttgagctttcttcatgcattgaatacggcagaatttctgacctgaaga
aatctagccttttccaaatttgctttaagaacattttgcaataaatttaatataataaaaggaaaaaa
cacatcaggctagaatttggaaccgattgttattaaaaatctcaagtctatcaatttaacttcaacaa
attacttaatttctgtgatggttaatttcatgtgtcaacttggctgggccgcagggtaccgagacatt
tggtcaaacattattctgggtgtgtttatgaggctgtttctggagagattcacatttgaatcagtaga
gggagcaaagccgattgttctcccttgtgtgggtgggtctgatccaatcaattgaggacctaagtcca
atcgattgaagacctaatcaaaaagcctgattaaaaggaactcctgcctgatagctaaagctggaaca
cccatcttttcctgcctttgagcttgaattgaaaccttgggtcttcttgagtcttaagcctccagttc
tggggctggaacttaacgtcattggctttcttggttctcatgcctttggactcagacaggaactacat
cattggctttcctgggtctccagcttgctgactgtaaatcttgggacttctccagattcgtaatgagc
caatttattacaataagtctctccctctctggtttcgagagagagagagagagagacagagagagaaa
tgagagcacaagaacgtgagtgtgagagtgccctaatataatttctctaaatatcactggttactctt
caaagttataaaattggtataaaaggtgacctcaatttttcatggagttaatgtatgaaagtcacaat
taaaaaggaagaattagttctggtgtcctgaaagttatttgaataaattaatatgctatggaggcttt
aaaatactatgaaaatttaatattgtattattcttagtgttgctatttttaaatagcactttttcttt
tcctttttttttttttttttttttttttttgagatggagtctcactctgttgcccaggctggagtgca
gtggcatgatctcggctcactgcaagctccactgcccgggttcacgccattctcctgcctcagcctcc
caagtagctgggactacaggcggccgccaccacgtccgggtaattttttgtatttttttagtagagac
ggagtttcaccgtgttagccaggttgttctcgatctcctgacctcatgatccacccaccttggcctcc
caaagtgctgggattacaggcatgagccaccatgcccggcttaaatagcactttttcttgtgagtcac
tttttaaatatttgtgcaaaccttgttgccattctactcaagctaatatcctaaaccgaggacattat
aacatttcaggagtcaaaacttcagacacttaacatagtatcctcaggttcatccatgttgtcataaa
tgacaggattttattcttttatatgactcaataatatcccattgcatatatatccaatattttcttta
ttcatccattattaaacacttaagttgattctatatcttggctattgtgaataatgctgcaataaaca
tgggaatgcagatatctctatgacatactgattttatttgctttgtctctgtccccagtagtggaatt
gctgtatcgtatggtagttctatttttaagttttcgaggaacctccataccgtcctccataatggatg
tactcatttacattcccaccaacagtgcataagggttcccttttctccatattcttgccaacactttt
tatcttttgtattttgataatagccattctaactggaatgagatgatatctcattgtggttttgattt
gcattttcctgatagtgatcttgaacattttttcatatgttgtattaactaagccaaacacagaaaga
caaatgcagcttgttctcattcatatgcacaatctaaaaacatcgatctcatagaagcagtaaatgga
cggtggtcaccaaagaatgggggaagtaggggaaaagcgagaatggggagaggattgtcaatgggtac
aaagtcacgattagaaaggaagaattagttctggtgtcctgttgcatagtatggagactattgtcaac
agtaaggtattgcgtatctcaaaacggctagaagagagggttttgaaggtttctaccccaaataaatg
gtaaatgtttgaggtgatatgctaattttcttgatttgatcaagtaaaggtcttaattgtttggcaat
taagactcatgaatacaaataaaggtcttaattatttggcaaagcatgctgagttttgtaaacaattc
agtagtgatttttgagaataggtcaatagcaaatattaattaaaatgtcttctatttatgacctacag
ctagatggtaaacagatagatgatagatagataactgatagataactaatagatgacagataaatgat
aaatagataaatatagataatcgagagagaatacctttcccttcacacacgtgcatataggcacactc
catttctatcatagttaccaggattcagacattttgtctcactatttttctcaatgtgaacatgcata
taggaatattatagtttttgttctgtgcccattttagttcgttttttaatatttcaggacaaaggcaa
tatggcggtttcactttgtttttcatttttgcttatactttttaaagctcagtgtagaaaagtttgaa
aatacacaaaagtattaaattaagacagctgggcacagtggctcacgcctgtaatcccagcacttcgg
gaggccaaggtgggtggatcacgaggtcaagagatcgacaccatcctggccaacatggtgaatcccgt
ctctactaaaaatacaaaaattagctgagcatggtggtgtgtgcctgtagtcccagctactcgggagg
ctgaggcaggagaatcgcttgaacccgggaggcagaggttgcagtgagccgggatcacaccactgtat
tccagcctggtgacagagcgagactctgtctcagaaaaaaaacaaaacaaacaaacaaaaaagcacct
atagtctttctcccataggttgccttcttaatgggttttacaccttttgatgttttcttgagttctgt
cccattagcaagtagtattgtacaaaaaaaattttatcatcttttatttaatattttattgatgttta
ataattagaattattttaaattttatatgtcattttaaaatgcaatacaatatagtaaactcccagat
gtgattgtaaataattaattattctcccattattgggcattgggactgcttccacattttggtcactg
cagtgaacatccttgtacatgaatctgtatgttgaagttgatttcattccacactccccttcattcaa
ggggctccaaccattctcgttttctttcagcttctttatatccaggcatataaagttccttcctgact
cgggagcgtcatacatgctgttttctccatctggataagtagttaattctgttcttctttgtgcatct
cccgtttcagtaacttcatctccaaagcctttccaggtcactttatctaaagttacaccataatcttg
caaatcctcaactattgagcattattagtctccgttatcattattctccattattctctgtgaaagca
tcccgtgattttcttttgtccctattaccacaatatgtgtttattccgtgtatgtacatctttgtttg
tttattgtttgtctatacctgcaatgaaatgcctaaggtcaggaactgtctgatgcaggatgcaatgc
gctcaataaatatttactgaacaaattaattcatttgctcagtcttgcaggcaaatggtacttctgta
tatttaaatatctaaaatgaaagcgttactcgttactgttggttgtcaatcaaaatttaaatgtcgat
gtttaagcgtgaaagacctctgtcaagttaatctgtacttacccaaaggctattatgtagaagcgaca
taaatattttcctaaatgttgattttcatattttaagaagacaatgaatgtttcaaagcattttcttc
tacacagctatttattctggagagtggggcatatgtttcttaatattgttaaaattggcaaggggata
ctgttgctatatacaaagaacacctaatcatcatgcagacgttttgtttctggctctcagttatgaaa
agcagagattttaaaaagttacctttatatgctaaattaggaatggcagaaggtaatattctaatgtt
tataagtggttcttctctgagtccttggtttctatgtttatgaattctctttttgaaagaaattatag
ttattattaccaggtctattcttttacattgtttctaattctatggtgatcttcaaaatagagtatca
attttaaatacttgggaatgaaattattcttcccatatcatttctttgtatggcatacattgtgattt
gttgtcccatcattgtttcagtatgacctgttactgcaaaaacatattgagataaatcatcccacata
ctctcggccaggacagacatcacactgttgcagcaacacttcagatgagccccattcaaccttgtgtt
tttatagagaaggatgccacatgtttatattcatttctgaagattggctcatattatttattgaaaca
tactagtttaaaaatctgtccatttatataacacctggtctatctacataacttgaattacataaata
taaaactaaacttcccctcttctccagtgtatagcttgcaagcaagtgcatgtgaaataaattaaagc
cttgtttgtgtttttttcatcatgtgagtacaagacttttcaataaaaatgaattacttttgaacata
tttgtttggacaacaaacaagagaaaagatctatttgattgatagtggacagaattttcattaagttc
aacagcagaaataccacaattgcatcattcaccttcgtgtatcaaaagaaaacagaaaattagatgtg
atgaactctacacaaatgttcactatgcatactttacccattaaatacattatcaagaatcatgtcag
catgacattctaatatagcagctttacaaaaacatgtaatctaatctagggatgctgttgtcctcttt
aaatcagcttcaaacatattctgggttgatatttctcattcttttttgatccacattgtttattcaca
taatgattatatttaactgaagataacagcattatcaaagtgaaagacaaaatagatgtttaatagga
aagtgagtatcgaatcatcttttttctaccaaaaacatctataattatgaagtatttggttaattatt
ttcacaataatttaaaagtgtacaacttgccgatttttttgtactttctacttttcatgtctcgcata
tatctctttaatatctaagtatttgagtcagaaaagagccagtaccgaataatgggaatctcactgaa
atgtgataacaatctggggcctggtcctgggacctttatctgcaggacaacttggacaaatatttaga
cccccaattcctcgtctttaccctaggaataataacacatttttctgacctcatacttcacgtggatc
tcaaatggaacaatcatctgatagcactttatgaagtatatgaaagcaataaattatcacaataagat
aattgcaattattctttggcatagtattagtgatgtctttatctgtctgacaaaatcaacatttctgt
atggtaactgcctttccttgttttaacagaagatcatgccagaaaagatgagtaggtagatacttaac
ttgttgttcctgaatctggaatgtattgcagatgtcccagactgatctttgttcttttttttccttac
aaatttcttttcacattgacagtgtgatatttctttaaatgtgcaatacatagctaaccttatttgtt
tgtgtttactaattaaaatatctaaactgcttaaaggagaaaattcagttttaagttttattgattta
tacccttcttcaatccacataggattagggtagtatgtaacaaaatttcaaactataaatgaaatatt
gagttttgtattaaggccaaggatgaggaaaaaaaaagtaagtatatatggaaaaagaatggtattga
atgggagttttgatggagcatgttgacatcatgataatacctattatctttatattctgaatgtcaga
acaaaattagagcaattttcccttatttccctacaatacgtctgtcttaataattctaagctttcctg
atttcagtagtaatctgtattttgcaaaaggcagcatgtttataagatatcaagtaaactaagtttat
ggaacttgtaacagcatttttaacaacatttctccctagatagttcatggtagacatgaatttattca
aaactagtatgtagaaaaataccattaacaaaagctctgaaattatattagaggagctgaataatgtt
acttgagaaagaataaaatgttatttatgatttttggtatcttttacccactatatatggccatatct
ctgaaaaactttagtaatatgtactaatgcaaatatggtagtaaattatgtctacaggtgctgatacc
atagtagataaagtatgataactttattttaaaatatcatatttaaataattaatatacagtactggg
aaagactattttatctattctctcactcttgaataaaaaaatccagaaaaaaataccttgttttggta
agattatatcaatttatttcccaaatgggtagagggttatttttttctgatcataaacgtatgtctct
tcattataaaaatccactaaaagtgatagaagaaaaccaaaagaataaatgtaaacaatgatgccatt
ttccaaaaatcaccttcgacatttttctggatattgatacagtctaaatctcttttcggaagactccc
tcctgtgtaggttccccaactactctgcaatcttatttcctcttgttctgttcttgtagaaaggagac
ccattgtcaccatgtcaaataacacaaaatggtgcacgtataagatcattgtctctgtccattatttg
ccagaggacctcaaactttttcaggtggtgggcaactggatgtcatgctgctccttgtacaacagaac
acaattcattatttatatggttatttcattttaagaaaatttaactttcattagctggaaaaaaaaag
aagtggtttttaagttgtttagaaatgtgaaattcaattttcatactgcaaaagagattcaactgcaa
acacaggcacacatgtctggtgtaagaacgagttgtcatacaaacccaaattagctgcctccacgttg
tctttgttaacaagtgtttgtttgctccttgttccatcattcagaaatgctctttagcaggaattgat
ggaacacagtcgcagtgacctcttcctgtctttaaaaatcgagatgacatttgcccatctgcagtgtt
aacatagttcctcaaagaccactgacagtggggtaggactgtattgcgcaagttctctcatttcccta
gaatataattggtccagggccagagattttagctcatttagagcagcaaggtgctcttttaaaattcc
ctcacctattttgggcttcatttcccttatacggttatgccttttccagtctgatgaacattctcctt
gacagagcagacaagcaaaaggagctgcacactgctgctttctgtgtcgtctctatccctaaccttct
cccttctgccccaatcagtgaaccttcgtctttctggttcttcttcctccaaatggaagtaaaaaggc
cctgaatgttgtctttaccattatcacgagcctcaattcattccaagctcagcttttcctcactgttt
atacagttctatattgttcttctaatatttgccctcagttctctgtccctcgtttcttcccatgttca
tactctattagaatctgagcacctttgaggttgtccatacagtggcacacatctttgttttatactca
ctgggatgatttgccattatattgtcaaaattttattctaaagagcttttacaggctttcttgagcca
ttttctcttgaaattcaagatcgttgaatctctacgctttttccttcttaatctaataaacatacacc
cccacatacacacgtgtgttcctgaaagacagatgccacttgactcgtcttatagattgtctaaattg
atcattgtgtgtggggataaaagggtgaattgtataatatccctgatggttcacgaagtctgttcctg
tataacctgattagtcttctgaactcttttaaattctgtctgcaaatgactgaggtttggcaatcagc
ctatttcagttagttgttttcttgcataagaagggtccatatgtactgtgtgaagtaagagagagaaa
gtacttagatttgctggatgccctgattgttagcatggctaaggtattgtgtaagtaaggagagcagt
taaaaatgatattgtttttatttcttaattgaggtaaaattttatataagatgaaacagacttatttg
ggagaggaggaagagtttgttcttacataacatttcaacctgtcatatttagttgagaacttcaatct
gtcaagatactttgtataatattcagattctgccatctaatatattttccacgctttcttactgggtg
tgacagtaacttatactgtggcaggtgtataagttagtaaagatattaaatgctcaatctgttaactt
ttgtgaagtggtcccactgataaagtgacacctcaataaaataaaaatttccattacctcagaaagct
ttttcatgctaccttccagtcaattcccagccccaataggcacctattcttctgatttatatcaccat
agattagttttgtctttttaaaaatttgtataaatgaaatcatacaaaatgtactattttgatcagca
tactacttttgagattcatccatgtaagtgtatcagctgttcattcctttattgatgattaatattct
attgtatagatataccacaatttatttatctattctccttttgatggacattcaggtggttttcagtt
tttggctgttatgaataagatgctgtggacatttgtgtacaagccatttgtgagcatatgttttcatt
tagtttgagtaactctgtagaagtggaatggctgggtgaaatgtttaaatttatgagatattgtcaaa
cagcacctaaacagttttctaaagtggttgtgccattttgcaatgccaccagtgatgatggagagttc
cagttactctacatctttgtcaatatttggtcttgtcagtcattttaatttttgctatcttacagaat
atgtaggtatattgttgtggttttaacttatattcctctgattactagcactattaagcatcttttca
tggatttattggacattcatatagattatgtgtgttgaagattattacctttatgattattgggtgaa
aatagtatcattttgaggtcattcatataacttgaagactgggaatgacagacattttcctgttttgt
ttcttttctttttactttatctgaagagtctactagaatgcagtgttgctgcctgagcagcagggcat
tagctttgtaaaagctctgttccttggcaaccccaccactaatatgaagtgcagaacatttgaattgt
ctttgaccagcttcagcatcagcactattttttttttttgctagacccctagtaggtatttaaaagta
cagaaatagaatttaatcatgctttttaccaaatgtgctatgctcttagagattctttcaacgtgcat
aaaaattctgcagtttcaccacataccagtaaaagaaactcagtcactcatttagccatttagtaaaa
agaacaaattaactgatgagcatagtggagacctcaaaggtaaagaagacaatgtccctgaaataaag
acaatcataaattttcaatcaaaataatgaaatttaggctgggcatggtggctcatgcctatgatcct
agcactttggaaggctaaggtgggaggattgtttgaggccaggagttcaagaccagcctcagcaaaaa
agtgagaccctgtctccacaaaaaaattttaaaaattatctgggtgtggtggtatgcaccggtggtct
cagctactcaagaggctgaggtggaggatcaccagagctcaggggttggagactacagtgagctatga
ttgtaccactgcactcaaacttgcatgacagaatgagtccttgtctctaataataacaaaatttaatt
tttatagactgtgaaaaaccattatgtagatacagttcaagtacagtatgattttataggatagataa
cttttgcttgaaaatgtattcccaatttataggatagataacttttgcttgaaaatgtattcacaata
gagttagtatttggggcacacctttatccatttaacaaacatgttttgagcactgccaggtagcaaca
cgttactaggcactagagtgagaaaagattacagttcctgctctcatggatctcatggtctagtcaac
tggaatgaaaggattacataagtagaggtaaagacacacatgatggaggatggagaatagtcaaaggt
ctggagaatgaccaggacgtcactgtgagttgtctaattgcactgaagcatggatgaagaattggaaa
gtcattgtaagaagcctaaaaaggtatctctcagggatgctatgaggttctgaatgttatgtacgcta
tttgggcttcaacaggcaggcactgagtattcagtataaatttttgagcagggaatccaccagaagaa
ctatgcatctggaggattaatctggaaagattgtgtagaatgttatgcagtgaaagagtctgagatga
aacagttaggagggtgtattaataacataggtgaagtgtaatgaataaccaggctggaggaaaagcaa
taacgatggaatcaaccgggcaagaagtataacaattaggatcagtaaaatagaatttggattggagg
aatgaaaaaaaaagggacaaaacaaagttgaactgctggtatccatactggaaaatacagatgtcatt
caaataaataatgtaatgaatataagaaaccagttttaggagtgaagtggatgttggcttgaaaatat
ttcctttgaggtttcagtcaaatgaaaaggtcctgaaatgctacgtggtagcctaagaaggaagcgtt
cctagagagaaaaaaattagaaaagatttacatttgataatttaatcttttccttcatacaagctaaa
ttgataagaaagtaaaacctatagttttcaccactcttttacaaatatccctaaccttttagatattc
acatgaataattgagaaaaatctaacagatgacttgcttatgtcatttgtctgctttatccttaggtt
cctctggcttatatattgttcaataaaatacagatcattgatattgtacaatgtactgataatgggga
gtgaatccatgcttgtgcattcttttttttttttttttttgatttgcagagggcgtgcccagtcaaca
agagaggcacaattgtttttatcatcacctcttctcatctaattccatgaaggagagtagtattacca
tacaacagataatgagttggaaaacaagaaacctaacctcagaacttaaggcttggggaaaaataaaa
gagtaatttgtgtttaatgcctgtataacttggcaagagggacatataaggcttagtgatgcccaaca
tgtgcttagatgtggattgttagttgatgtcttgggggttctgtaatctaagctaaatgctcaaaatc
aattaattgatgttagacacagagatctgctttgatccctctttatcgtatttctaggccttcccatt
ctcaagagcctgagaaacgacagctttccttaataacttgttatttgtggtaggagatgaaactttga
taaaaacacaattatttttaaatgtctctttttcactctaggctgttgtatgtatttcaaaaagttac
ttttgaccctttccagaatgagaaagcaatcaagaagattataatatcttgcttagttttctgctcaa
tttatcaacaaatatttcttaagcaattattaagctgagcagtgctcagcgctgtacttggtgatata
ggaaatggggaaaagactgtctttaaggcctttataatagtaattacctcaacttgtctgtttctttt
ccttaccatttcgccaaattcattgatctatcttgttctcaaagcaatcgccatagttatattgtaac
acagcattttctagggtgtccccattaagttgagagtgttgacaagaaaatacaagcttatttatcat
tgtaaaacttgagacacctagtagttaccctaaattaaatatttgttggagtcagtcacactaaagag
aacacttactgcattgaacaatttacctacattagacagcatttaaagactatgccacagcaaaggcc
catggaattcttgtgaacacagaatagaagtgtattaaggaacaagcttaattctgttctcttaaagc
acaacactttctcaaaacatattttgaaatcacctttgaccattttttttaactaataggtgggtggg
agttagggtaggaaaacacaagcagcttcatcaaaacgatattctattttcttcaaatttgtggggaa
tcatacggcctctcaattttctacattatgctaattatgatattaatctctctgccagcaaatgaaaa
taatacatattagatgtagcaaatgtcaataatgacaaaattagtcatcatgcagatactcagggatt
cccaaaatatgtttggattatgattgctagctttgagtttgcccagaatcgtttcaataaaaataagg
gactcaaacacatttggagcaaaactcacatcataaattttagacatagctctgccaataatgctctc
agttatattttcagtcctaatatttcctctgagttccagaccagtatcttcaactgtctgattgatac
tctctccttcatttctgtctccaatgcattaagtcctgtgtatttactttccaaatgccacttggttc
catgcacttctctccatttctgccactgactcctcctcaatccaagcgaccatctttcctcactttaa
ctaccatgatatctcctgcttggtctccttacttctattcccgggctcctccaatccattcatcctcc
agcagagaatgatgactagcaccttccacagtgtctggctaataggaggtatccaatcaataattgac
ttacagagtgaaaatataggcatggcaaataccagtagagaactacagggttttagaaccaatgacat
tagatacttccatcaaatatttacagtgtataatcaagttgacttgcacattgtcttatttttgaaaa
acaattttgttggctttttctatatgcacacatacatattgtatcaccctctacccgccaaatggctt
ttgaagaagtatttatgtggctccaaattgataatacctctagagagaagagaaattagaaattttaa
aatgacctatgcttcctttcgaatatcacgtcctgagacagtgttttttgagttacgtgcaatatgtt
ccacgatgaaacatttaatgtgttcagaggcatgctagtaatcatgtagaaagaattttatgcctgaa
gtcacatgttctataaccaggatcacttaataagaaaacaagtacagctgtggacaagatgccttttt
atcagggaaaggccaatttgttttctttgcaaatctaagtaaatggagagaaaaacacagcccttaaa
tgttttctatttgtcctgaagttctcatgaatgagttagaaggcgagaaggattaaataaatccttga
acgtagagagagctaacatttattttagcaaactaaaacctattcgctttgcaaagttctgttctgta
ctttgtaacaacagttttctttaaaacaagagccaccaattcaaatgcctttacagaatgattgaatg
ctttcatgccccacctaaaggcattcaaatcattaatcaaacaaagttctaacgccaaaacatgtctg
ggaccagatttaaaatgtagccctcagtttcagagggcaaaaacttaacatatttatattttcctcac
tttaggtaacactgtattgaatctctgcttgaaattgaggagcacgtgattttttctttttggcccag
ggcagcatttcttggaagagaaagaaaaacaacccaagatacccttacaaaacatgtagtacttaaag
ctctttatgatgaattaattttggtatacacattaatagcagtgataataacaaatctatatatatat
atataattgatatgaataagataaatacatcaaaaggaaatttcattacaatttgatattaggtaaat
gtcccattaaaataaattgctactgtacataattttccttcagttcattggcaggatgtttgctttgg
aaaataaacagtctatttctagttttagaaggaattctcattattcttttatagcaaccattatcagg
agcagatgggaaattgtaccaagagcatatctactattatacctcacaggaaaaagagagtattaaat
gaaatctaacaaggcctgctcctgactctagttcctgtaacaaatgaacacacacatttgtatggttt
cagcatttgtattagtaaggtacaataaatgtttactgaaattgaaaaaaaaaaagataacaggagaa
agaagaggctaaaaaggtgcattttatttctgatcgttcattgtaaagactgctcctttttaaaataa
tcaaattttattttatatacagagggtacatgtacaggcttgtcacaggggaatagcgcatgatgctg
aggtttggggtacagatctcatcacccaaacagtgagcatagtacctacctgatgagtagtttttcaa
ccaatgcgcaccctccctccttcccacatctactagtccgcggtatctgttgttcgcatatttacgtc
catatatgctctatgtttagctcccacttataagtgagaacatatagtgtttgtttttcctgttcctg
cgttaatttgcttatgattatggcctccaactgcatccgtgcttccgcaaaggacatgatttcattct
ttttatgactatgtagtatttcatggtgtatatgtaccacattttctttatccaatctaccattgttt
cacaactagatggattccatgtctttgctattgtgaatagcacaagacaggacctttttatttgactg
agttccttgcaaattactaataaaagatctggaggtccttagttaaaagttgaatctgtagtgccgtt
caaatttagagatgtattttctgttcaagagaagaaagccctcattcggtcatgcttaatattcagct
gtaaagtccaaaacatatgagaatgacacaaatggaaacattttataaatacctatacaaaggagggg
cacttagttcccctaggcctcttaaaagtcctctagaaagagggtacttttatgctaactattaaaga
tgagtaacgaatttgtcctatacaacttaacagtatcgtcaaggaagtagaaagttactcagttttac
tgggcattggagctaagcttgaaagtgaggaggagaagcggcaggagacggagccgagaaggcagtgg
ggagaagaggaggatggtcctttccatgctccctgttgtactaacatgtttggatattatcttatact
tcatatatggactggattcttgtccttctcattctgagctctccttgaccttgattcttacctcctat
aactttcattctttctttactcaaaaaaaggccatttatttcagccatttttcactgttttcttatcc
ttcctagttgcttttctatactatttttccactcttttttttttctatactattttgcccttctctcc
attttcctaactgctagatttccccaattttagccatctttcaattgttctgactatcctcaggtgct
cccacaaggttatcagaccttccaccaagacggaatccctcagtctatggacaggctaagttgaatgg
gtcctggtgctgtgcttagcatatgccttgagtatttgtgcatttattttgcttctttacaaaaatcc
atcatccgatagaagttgaaagaaacttgctgaagcacattaaaatctctgaaaacagtattggctat
attttctaataattagcatgactggttaacttgctttatttatcattgaaaaaagtatcagaaactgt
atatcaaactcctgaattcttggcactgacgaagagacacaatgagaatgaccttaggataaaaaaac
aagataaagcaccatatttgtaggaaattgcaccataaaagtctgtttcacaactctcccaaatttca
ttttattacatcttttctcttgaccaatcagtaaactcggttaatgatttacctgtctcaaaataatt
catgaacaaaattacaagtaaatctcagtattggattcttgaaacatctccttgttcaatgaagtttc
ctttttcttccctctatttccctgtatttatcttttcttccagttgcattttatctcttctgtttttt
tatcttgctccctagtttgtgattttttgccaattttttatttcctacataattcatccaatctgtca
ttgtacaatttcttataactgcttcttagcttattccttttcttcatttgtcacattctatttttcat
ctattgtgttttcatgcagttttggaaagttttacaaatagacttttaaaaaaatgtacgtaatgttt
tcatagaaaaggtagtggtttctttttcttatatccttccctgtataaaaataaaaatgtagcagttc
tttctttgcctatgtttcctctttccttcccccaatttgaccagacttgaaggacttagatatgtaac
agtgttattttctataatttaggaacagcttttgacttaaaaagcagaagagaagttgaaaataatat
agtaattctacatgtccttcctgcttcccaactctctgcacatgtttgtaacctcccctttctttttt
agtgtatctctttcatatacctttgtccccagaaattctgattcagtagacttagaatggaattctgg
gcttttatattttgaaaagctccccacgggagttagatatgcacttcttattaagaatgaatgcttaa
tattggaatcaaaacacaataagctttctaactatgatgaataatccaacagatttaattatgatttt
ctttttgtccagaaccaagactagatgttaattgccagagaaatagataagaatgcctatgacagcag
tacattaatatgatatcaaagcttggaaattttattggtaatgaataattcagtacttaaaatattta
gaagctatagaattaaaattaattaatgttgttcactgtgtgaataaagttgattgagattttacatt
taattttgtaaacccagtgttatcttttccagctcagaaaacaccacatacaagctactactttctgt
tttgatcccttatttttctttcttatgctttatcactgaaaactctccttgagcaggccatgcactgt
aaatatttctcctggttgcaaaaccttctcatacaaatgcagtagactgtgtaatgagctcttctttc
acaaaattaaaaaaacctgaaagccctgatttgcgattctatacaaatgagatttagatctaacaatt
ttaaattattgcttcactcttagctgttcaattctatctcttatttgggaaaccgaaataataaaacc
attgctgattccacaattaggttgtaaaagtcaccgtagccatcagccatgaagcaaaagtgccaaga
tcaaaactacaaagcaaagaggctgagataaaaatgctgcagcattagtttatagcattataagcagc
aataagaattccttgattgcttaacaaagactcaaaaggcatttactccattaccttacaactcaaag
aggtattcctggaccagcagtattggcatttttttgaagtttgtaggaaatgcagaattttggtgcct
ccacggacctaatgcagcagaacttgcagtttagtaagatctccaggagatttgtatgcgcattaaag
tctaggaagcaccgctatggtatacatctgatgtgtgcccatgcattttttaaaagtatgaagtaata
gttgtaagtattggacactcttgaaggaacaaataagagccatggtctttactctctaaatacctccc
tgacatctatgttttaggcaaaatttttttcccatttcagtagtcactgatgcttgcacgatgcagtt
tattccaaaacaatggtgattctcatgtaatagttcatgttgccttaataatttacgttgcctcaagt
tctctgcccaggccccaatatacaccgagggctgtactcctcccctaacgcctgctctcatacagtgg
catagagcccagttttatgctcttggtcacatcatggagattgcacaccacaggctttaacttctgcc
gtactctcactgcctctaaccctccatatgcctaagttctacgattctttaaattccaaattgaccca
gaagtctcctccgctcatccttttcactgagatcatccctcttctggcctaccatttgttgatcacct
tgctttttttttatcctactgtatgtagtataacaaattatcacttgcaactgtgtcttattttttca
actagattatgtactgcctaagacctagaaaattgtgcttatttatttgaatctctaggaggatcagt
aatgggtattaatactaatgactccatggtgatgatgagcctgaacttcctcccttcctttctttcta
cctctctcctttcctcccttcttttcttcctccattccttcctctcttcctccctccgcttcttcccc
acttcccttattcatagattcatgcgttcactcagcaaatgcttactgaaaccttccatgcatcagac
attgtactaaacaataggaaactatcatgaataagacacaatatctgacctcaaagaatttatgatat
aaaagtaatggcataaaccgtgattacttttgcaccaacctaatatatagacacagtttgttatgact
ggtgtctctattactaagcaatgactgtcacatgcaacgctgatctgaacaggtggtaaagagtgaga
tgtaagcaatggagcaaagccaactagttacaaggaaatatcacatgtttactagagcacatctcatg
ggcattcaagagagtatggccaggacagcttgtgaatagttcagtaactgtgcatagttttatattca
ttgtgaggcaccgtgtcaccggtttgctgatttacagagtattttaattgctaactgtatgctaccaa
aatttccagtattcgaaaataattttgcttgaatgtagaaaaagaaaaaagccaagaaatgtatgtga
aacgagagtctaagggagctttacctcagtctcagaaaacatgcattccttccttcatttaggaagca
tgtactggggtctactgtcagcttgctattgtgtcaaggagtaggagaatacaaaaatattagagaat
atgaatcacatctattaggagagttttctacatacgcacattattctgtcagtgacataaggatttga
gtcattcagatttaaatacggtaggtacctcaagttctcagatattatttcattttctaaggttcgta
tttagttaatatgttattttaatggccttacaaattctagattatcttttttaaaaagttaaatagaa
cgtaattgccatttttatttaatggtaaaaagcatttttgtttttgtgtgtacttggttgtaatattc
tccttttcaattgagctatttttctgatactttactcttaaaatttcattcaggaaaaaagtaaacaa
tatttaagcttgacaatcataaaaatgctctggtgactatagattattttaaaatttattactgtagc
ttagggatatcttgatgggatgctcctgaaagcaattaattctcagttttttgtggcttctaatgcaa
aatacattgacgcagacagaatttgaaatgaattttcttctaatatagcaattaattttatttaaata
tctctagagtttttttttaatactgtgactaacctatgtttgttctttttcacctctcgtatccacga
tcactaagaaacccaaatactttgttcatgtttaaattttacaacatttcatagactattaaacatgg
aacatccttgtggggacaagaaatcgaatttgctcttgaaaaggtttccaactaattgatttgtagga
cattataacatcctctagctgacaagcttacaaaaataaaaactggagctaaccgagagggtgctttt
ttccctgacacataaaaggtgtctttctgtcttgtatcctttggatatgggcatgtcagtttcatagg
gaaattttcacatggagcttttgtatttctttctttgccagtacaactgcatgtggtagcacactgtt
taatcttttctcaaataaaaagacatggggcttcatttttttttgcctttttggtatcttacag

| DNA encoding gRNA | gRNA |
|---|---|
| GGGGCTCCACCCTCACGAGT | GGGGCUCCACCCUCACGAGU |
| (SEQ ID NO: 157) | (SEQ ID NO: 171) |
| GCACAAAAGTCAAATCGGAA | GCACAAAAGUCAAAUCGGAA |
| (SEQ ID NO: 158) | (SEQ ID NO: 172) |
| GATTTCAATATAAGATTCGG | GAUUUCAAUAUAAGAUUCGG |
| (SEQ ID NO: 159) | (SEQ ID NO: 173) |
| GTGAGGGCTCCACCCTCACGA | GUGAGGGCUCCACCCUCACGA |
| (SEQ ID NO: 160) | (SEQ ID NO: 174) |
| GAAGGATTGAGGGCTCCACCC | GAAGGAUUGAGGGCUCCACCC |
| (SEQ ID NO: 161) | (SEQ ID NO: 175) |
| GGCTCCACCCTCACGAGTGGG | GGCUCCACCCUCACGAGUGGG |
| (SEQ ID NO: 162) | (SEQ ID NO: 176) |
| GTGAGGGCTCCACCCTCACGA | GUGAGGGCUCCACCCUCACGA |
| (SEQ ID NO: 163) | (SEQ ID NO: 177) |
| GGGCTCCACCCTCACGAGT | GGGCUCCACCCUCACGAGU |
| (SEQ ID NO: 164) | (SEQ ID NO: 178) |
| CACAAAAGTCAAATCGGAA | CACAAAAGUCAAAUCGGAA |
| (SEQ ID NO: 165) | (SEQ ID NO: 179) |
| ATTTCAATATAAGATTCGG | AUUUCAAUAUAAGAUUCGG |
| (SEQ ID NO: 166) | (SEQ ID NO: 180) |
| TGAGGGCTCCACCCTCACGA | UGAGGGCUCCACCCUCACGA |
| (SEQ ID NO: 167) | (SEQ ID NO: 181) |
| AAGGATTGAGGGCTCCACCC | AAGGAUUGAGGGCUCCACCC |
| (SEQ ID NO: 168) | (SEQ ID NO: 182) |
| GCTCCACCCTCACGAGTGGG | GCUCCACCCUCACGAGUGGG |
| (SEQ ID NO: 169) | (SEQ ID NO: 183) |
| TGAGGGCTCCACCCTCACGA | UGAGGGCUCCACCCUCACGA |
| (SEQ ID NO: 170) | (SEQ ID NO: 184) |

1. A CRISPR/Cas-based genome editing system comprising one or more vectors encoding a composition, the composition comprising:
(a) a guide RNA (gRNA) targeting a fragment of a mutant dystrophin gene, wherein the gRNA hybridizes to a target sequence within intron 51 or intron 44 of the mutant dystrophin gene;
(b) a Cas protein or a fusion protein comprising the Cas protein; and
(c) a donor sequence comprising a fragment of a wild-type dystrophin gene, wherein the donor sequence comprises exon 52 of the wild-type dystrophin gene.

2. A CRISPR/Cas-based genome editing system comprising:
(a) a guide RNA (gRNA) targeting a fragment of a mutant dystrophin gene, wherein the gRNA hybridizes to a target sequence within intron 51 or intron 44 of the mutant dystrophin gene;
(b) a Cas protein or a fusion protein comprising the Cas protein; and
(c) a donor sequence comprising a fragment of a wild-type dystrophin gene, wherein the donor sequence comprises exon 52 of the wild-type dystrophin gene.

3. A CRISPR/Cas-based genome editing system comprising one or more vectors encoding a composition, the composition comprising:
(a) a guide RNA (gRNA) targeting a fragment of a mutant dystrophin gene;
(b) a Cas protein or a fusion protein comprising the Cas protein; and
(c) a donor sequence comprising a fragment of a wild-type dystrophin gene.

4. A CRISPR/Cas-based genome editing system comprising:
(a) a guide RNA (gRNA) targeting a fragment of a mutant dystrophin gene;
(b) a Cas protein or a fusion protein comprising the Cas protein; and
(c) a donor sequence comprising a fragment of a wild-type dystrophin gene.

5. The system of claim 3 or 4, wherein the gRNA hybridizes to a target sequence within intron 51 or intron 44 of the mutant dystrophin gene.

6. The system of claim 1, 2, or 5, wherein the gRNA hybridizes to a target sequence within the polynucleotide sequence of SEQ ID NO: 128 or SEQ ID NO: 156.

7. The system of any one of claims 3-6, wherein the donor sequence comprises exon 52 of the wild-type dystrophin gene.

8. The system of claim 1, 2, or 7, wherein donor sequence comprises the polynucleotide sequence of SEQ ID NO: 53.

9. The system of any one of claims 1-8, wherein the fragment of the wild-type dystrophin gene is flanked on both sides by a gRNA spacer and/or a PAM sequence.

10. The system of any one of claims 1-9, wherein the gRNA targets an intron that is between exon 51 and exon 52 of the mutant dystrophin gene.

11. The system of any one of claims 1-10, wherein the donor sequence comprises multiple exons of the wild-type dystrophin gene or a functional equivalent thereof.

12. The system of any one of claims 1-11, wherein the donor sequence comprises one or more exons selected from exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, exon 72, exon 73, exon 74, exon 75, exon 76, exon 77, exon 78, and exon 79 of the wild-type dystrophin gene or a functional equivalent thereof, or wherein the donor sequence comprises exons 52-79 of the wild-type dystrophin gene or a functional equivalent thereof, or wherein the donor sequence comprises exons 45-79 of the wild-type dystrophin gene or a functional equivalent thereof.

13. The system of any one of claims 1-12, wherein exon 52 of the mutant dystrophin gene is mutated or at least partially deleted from the dystrophin gene, or wherein exon 52 of the mutant dystrophin gene is deleted and the intron is juxtaposed to where the deleted exon 52 would be in a corresponding wild-type dystrophin gene.

14. The system of any one of claims 1-13, wherein the gRNA binds and targets a polynucleotide sequence comprising:
(a) a sequence selected from SEQ ID NOs: 29-51, 87, 157-170;
(b) a fragment of a sequence selected from SEQ ID NOs: 29-51, 87, 157-170;
(c) a complement of a sequence selected from SEQ ID NOs: 29-51, 87, 157-170, or a fragment thereof;
(d) a nucleic acid that is substantially identical to a sequence selected from SEQ ID NOs: 29-51, 87, 157-170, or a complement thereof; or
(e) a nucleic acid that hybridizes under stringent conditions to a sequence selected from SEQ ID NOs: 29-51, 87, 157-170, a complement thereof, or a sequence substantially identical thereto.

15. The system of any one of claims 1-14, wherein the gRNA binds and targets or is encoded by a polynucleotide sequence selected from SEQ ID NOs: 29-51, 87, 157-170, a complement thereof, or a variant thereof.

16. The system of any one of claims 9-15, wherein the gRNA spacer comprises a sequence selected from SEQ ID NOs: 29-51, 87, 157-170, a complement thereof, or a variant thereof.

17. The system of any one of claims 1-16, wherein the gRNA comprises a polynucleotide sequence selected from SEQ ID NOs: 64-86, 88, 171-184, a complement thereof, or a variant thereof.

18. The system of any one of claims 1-17, wherein the gRNA binds or is encoded by a polynucleotide sequence selected from SEQ ID NOs: 35, 40, and 44, or wherein the gRNA comprises a polynucleotide sequence selected from SEQ ID NOs: 70, 75, and 79.

19. The system of any one of claims 1-18, wherein the donor sequence comprises a polynucleotide sequence selected from SEQ ID NOs: 53-56, 154, and 155.

20. The system of claim 19, wherein the donor sequence comprises a polynucleotide of SEQ ID NO: 55.

21. The system of claim 19, wherein the donor sequence comprises a polynucleotide of SEQ ID NO: 56.

22. The system of any one of claims 1-21, wherein the Cas protein is a Streptococcus pyogenes Cas9 protein or a Staphylococcus aureus Cas9 protein.

23. The system of any one of claims 1-22, wherein the Cas protein comprises an amino acid sequence of SEQ ID NO: 18 or SEQ ID NO: 19.

24. The system of any one of claims 1, 3, and 5-23, wherein the vector is a viral vector.

25. The system of claim 24, wherein the vector is an Adeno-associated virus (AAV) vector.

26. The system of claim 25, wherein the AAV vector is AAV1, AAV2, AAV3, AAV4, AAV5, AAV6, AAV7, AAV8, AAV9, AAV-10, AAV-11, AAV-12, AAV-13, or AAVrh.74 vector.

27. The system of claim 26, wherein one of the one or more vectors comprises a polynucleotide sequence selected from SEQ ID NOs: 57-60 and 129-130.

28. The system of any one of claims 1-27, wherein the molar ratio between gRNA and donor sequence is 1:1, or 1:5, or from 5:1 to 1:10, or from 1:1 to 1:5.

29. A recombinant polynucleotide encoding a donor sequence, wherein the donor sequence is flanked on both sides by a gRNA spacer and/or a PAM sequence, and wherein the donor sequence comprises one or more exons selected from exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, exon 72, exon 73, exon 74, exon 75, exon 76, exon 77, exon 78, and exon 79 of a dystrophin gene.

30. The system of any one of claims 1-28 or the recombinant polynucleotide of claim 29, wherein the dystrophin gene is a human dystrophin gene.

31. The system or the recombinant polynucleotide of claim 30, wherein the system results in a dystrophin gene that encodes an in-frame transcript comprising an exon 51 joined with an exon comprising a sequence of SEQ ID NO: 53 or SEQ ID NO: 55, and with an intron therebetween.

32. The system or the recombinant polynucleotide of claim 30 or 31, wherein the donor sequence comprises a polynucleotide sequence comprising exons 52-79 of the human dystrophin gene.

33. The system or the recombinant polynucleotide of claim 32, wherein the donor sequence comprises the polynucleotide sequence of SEQ ID NO: 55 or SEQ ID NO: 56.

34. The recombinant polynucleotide of claim 29, wherein the recombinant polynucleotide comprises a sequence selected from SEQ ID NOs: 57-60.

35. A vector comprising the recombinant polynucleotide of any one of claims 27-32.

36. A cell comprising the recombinant polynucleotide of any one of claims 29-34 or the vector of claim 35.

37. A composition for restoring dystrophin function in a cell having a mutant dystrophin gene, or the composition comprising the system of any one of claims 1-28 or 30-33, or the recombinant polynucleotide of any one of claims 29-34, or the vector of claim 35.

38. A kit comprising the system of any one of claims 1-28 or 30-33, or the recombinant polynucleotide of any one of claims 29-34, or the vector of claim 35, or the composition of claim 35.

39. A method for restoring dystrophin function in a cell or a subject having a mutant dystrophin gene, the method comprising contacting the cell or the subject with the system of any one of claims 1-28 or 30-33, or the recombinant polynucleotide of any one of claims 29-34, or the vector of claim 35, or the composition of claim 37.

40. The method of claim 39, wherein the method results in a dystrophin gene that encodes an in-frame transcript comprising an exon 51 joined with an exon comprising a sequence of SEQ ID NO: 53 or SEQ ID NO: 55, and with an intron therebetween.

41. A method for restoring dystrophin function in a cell or a subject having a disrupted dystrophin gene caused by one or more deleted or mutated exons, the method comprising contacting the cell or the subject with the system of any one of claims 1-28 or 30-33, or the recombinant polynucleotide of any one of claims 29-34, or the vector of claim 35, or the composition of claim 37.

42. The method of claim 41, wherein the method results in a dystrophin gene that encodes an in-frame transcript comprising an exon 51 joined with an exon comprising a sequence of SEQ ID NO: 53 or SEQ ID NO: 55, and with an intron therebetween.

43. The method of claim 41 or 42, wherein dystrophin function is restored by inserting one or more wild-type exons of dystrophin gene corresponding to the one or more deleted or mutated exons.

44. The method of any one of claims 39-43, wherein the subject is suffering from Duchenne Muscular Dystrophy.

45. A genome editing system for correcting a dystrophin gene, the system comprising a donor sequence comprising exons 52-79 or exons 45-79 of the wild-type dystrophin gene.

46. The genome editing system of claim 45, further comprising a nuclease selected from homing endonuclease, zinc finger nuclease, TALEN, and Cas protein.
